(19) United States
(12) Patent Application Publication
(10) Pub. No.: US 2023/0242919 A1
(43) Pub. Date: Aug. 3, 2023

(54) ENZYMES AND REGULATORY PROTEINS IN TRYPTAMINE METABOLISM

(71) Applicant: CB Therapeutics, Inc., Carlsbad, CA (US)

(72) Inventors: Laura Flatauer Peiffer, San Diego, CA (US); Jacob Michael Vogan, San Diego, CA (US); James Lee Wade, San Diego, CA (US); Tyrone Jacob Yacoub, Murrieta, CA (US); Kirsten Tang, South Lake Tahoe, CA (US); Rachel Nadine Burnett, San Diego, CA (US)

(73) Assignee: CB Therapeutics, Inc., Carlsbad, CA (US)

(21) Appl. No.: 18/000,931

(22) PCT Filed: Jun. 4, 2021
(86) PCT No.: PCT/US2021/036031

§ 371 Date: Dec. 6, 2022

Related U.S. Application Data
(60) Provisional application No. 63/035,692, filed on Jun. 6, 2020.

(51) Int. Cl. C12N 15/52; C12N 15/81; C12N 15/82

(52) U.S. Cl. CPC C12N 15/52; C12N 15/815; C12N 15/8254; C12Y 201/01049; C12Y 207/04003; C12Y 401/01028

(57) ABSTRACT

Provided are non-naturally occurring nucleic acids comprising a sequence encoding an enzyme or regulatory protein in tryptamine metabolism. Also provided are a recombinant microorganisms expressing the enzyme or regulatory protein. Methods of expressing the enzyme or regulatory protein are additionally provided.

### CROSS REFERENCE TO RELATED APPLICATION

This application is a 371 National Stage entry of PCT Application Serial No. PCT/US21/36031, filed Jun. 4, 2021, which claims the benefit of U.S. Provisional Application No. 63/035,692, filed Jun. 6, 2020, and incorporated by reference herein in its entirety.

### INCORPORATION-BY-REFERENCE OF MATERIAL SUBMITTED ON A COMPACT DISC

The Sequence Listing, which is a part of the present disclosure, includes a computer readable form and a written sequence listing comprising nucleotide and/or amino acid sequences of the present invention. The sequence listing information recorded in computer readable form is identical to the written sequence listing. The subject matter of the Sequence Listing is incorporated herein by reference in its entirety.

### BACKGROUND OF THE INVENTION

### Field of the Invention

The present invention generally relates to the production of substituted indoles, e.g. N-methyl-L-tryptophan (NMTP), N,N-dimethyl-L-tryptophan (DMTP), and N,N,N-trimethyl-L-tryptophan (TMTP), and related tryptamines, e.g. N-methyltryptamine (NMT), N,N-dimethyltryptamine (DMT), and N,N,N-trimethyltryptamine (TMT), in a modified heterologous microorganism.

### Description of the Related Art

Mental health problems, which may also be referred to as mental illness or psychiatric disorder, are behavioral or mental patterns which impair the functioning of individuals across the world. Such mental health disorders include: personality disorders, anxiety disorders, major depressions, and various addictions. Indolic and tryptamine-based compounds similar in structure to the endogenous neurotransmitter serotonin have been increasingly evaluated for treating mental health problems. In contrast to anxiolytic medicines, usage of substituted indoles and methylated tryptamines, such as N,N-dimethyltryptamine does not lead to physical dependence.

The chemical synthesis of hydroxy, methoxy, phosphorylated, prenylated, and halogenated substituted tryptamines and indoles typically involve tedious techniques of organic chemistry. Often, reproducibility is elusive and the solvents used during the syntheses of substituted tryptamines are environmentally toxic. Decarboxylations and selective methylations can be difficult to obtain via the techniques of organic chemistry. Further, the yields and purity of the intermediates for obtaining the target molecules can be low, where, for example, the starting molecule is L-tryptophan and the target molecule is N,N-dimethyltryptophan (DMTP), bufotenine, 5-MeO-dimethyltryptamine (5-MeO-DMT), 7-dimethylallyltryptophan, psilocybin, aeruginascin, among others.

The present invention provides for producing substituted tryptamines and indoles in recombinant microorganisms, providing for a more environmentally benign and higher yielding processes for production of those compounds.

### BRIEF SUMMARY OF THE INVENTION

In some embodiments, provided is a non-naturally occurring nucleic acid comprising a sequence encoding an enzyme or regulatory protein in tryptamine metabolism, where the enzyme or regulatory protein is an N-methyltransferase (INMT, PsiM, TrpM), a tryptophan decarboxylase (AADC), a tryptophan hydroxylase (TPH), a tryptamine 4′ hydroxylase (T4H), a tryptamine 5′ hydroxylase (T5H), a truncated cytochrome p450 reductase (T4H-CPR, T5H-CPR), an hydroxytryptamine O-methyltransferase (IOMT or CaffOMT), an N-acetyltransferase (NAT), a deacetylase (DAC), a hydroxyl tryptamine kinase (PsiK), a tryptophan synthase (TrpS), a toluene monooxygenase (TMO), an aminotransferase/methyltransferase fusion (ATMT), a phosphatase, an oxidase, a dimethylallyltryptophan synthase (DMAT), an isopentenyl-diphosphate isomerase (IDII), a tryptophan halogenase (TrpHalo), an aspartate oxidase/quinolinic acid synthase fusion (AOQS), a tryptophan importer (TAT2), a methionine importer (MUP1), or a SAMe importer (SAM3).

Also provided is an expression cassette comprising any of the above nucleic acids with a promoter functional in a recombinant microorganism.

Additionally provided is a recombinant microorganism comprising the above expression cassette, that expresses the enzyme or regulatory protein encoded therein.

Further provided is a non-naturally occurring enzyme or regulatory protein comprising an amino acid sequence encoded by any of the above-identified nucleic acids.

### BRIEF DESCRIPTION OF THE SEVERAL VIEWS OF THE DRAWINGS

FIG. 1 depicts the chemical structures of tryptophan and tryptamine, including various modifications which are performed by the enzymes disclosed within.

FIG. 2 depicts various substituted indole compounds in the tryptamine and tryptophan pathways utilized in the present invention. Panel A depicts the indole ring structure with positional numbering, and tryptophan and tryptamine. Panel B depicts examples of hydroxy modified tryptophan and tryptamine. Panel C depicts the 5-hydroxy indole ring structure with positional numbering, and examples of modified 5-hydroxy tryptamines. Panel D depicts the 4-hydroxy indole ring structure with positional numbering, and examples of modified 4-hydroxy tryptamines.

FIG. 3 depicts biosynthetic pathways utilized herein. Panel A depicts the biosynthetic pathways to tryptophan and genetic manipulations to increase tryptophan flux toward modified indoles and tryptamines. Panel B depicts the biosynthetic pathways to the methyl donor, SAMe and genetic manipulations to increase SAMe flux toward modified indoles and tryptamines.

FIG. 4 depicts enzymatic reactions utilized herein. Panel A depicts SAMe usage by INMT for methyltransferase activity. Panel B depicts BH4 usage by TPH for hydroxylase activity. Panel C depicts SAMe usage by INMT for methyltransferase activity on hydroxy tryptamine. Panel D depicts SAMe usage by IOMT (or CaffOMT) for methyltransferase activity. Panel E depicts NAD(P)H usage by T5H for hydroxylase activity. Panel F depicts acetyl-CoA usage by NAT for acetylation activity.

FIG. 5 depicts routes of modification of tryptamine by combinatorial usage of INMT, T5H, and IOMT enzymes.

FIG. 6 depicts routes of modification of tryptophan by combinatorial usage of TrpM, TPH, and IOMT enzymes (Panel A) and example branch points where modified tryptophan becomes modified tryptamine via use of the AADC enzyme (Panel B).

FIG. 7 depicts (A) routes of modification of serotonin by combinatorial usage of INMT and IOMT enzymes; (B) conversion of 5-HTP to serotonin by the AADC enzyme; (C) conversion of serotonin to N-acetylserotonin by the NAT enzyme, and N-acetylserotonin conversion to melatonin via the IOMT enzyme; (D) conversion of serotonin to 5-MT by the IOMT enzyme, and 5-MT conversion to melatonin via the NAT enzyme; and (E) conversion of melatonin to 5-MeO-tryptamine by the DAC enzyme, and subsequent N-methylation by INMT to generate compounds such as 5-MeO-DMT.

FIG. 8 depicts (A) halogenation of tryptophan and tryptamine on the indole ring by the TrpHalo enzyme; (B) example route to halogenated DMT via combinatorial use of TrpHalo, AADC, and INMT enzymes; (C) prenylation of tryptophan and tryptamine on the indole ring by the DMAT-IDI1 fusion enzyme; and (D) example route to prenylated DMT via combinatorial use of DMAT-IDI1, AADC, and INMT enzymes.

FIG. 9 depicts (A) a modified host organism expressing gene combinations with TPH, AADC, and TrpM enzymes to convert tryptophan into various hydroxy tryptamines; (B) a modified host organism expressing gene combinations with TPH, AADC, TrpM, and IOMT enzymes to convert tryptophan into various methoxy tryptamines; (C) a modified host organism expressing gene combinations with AADC, T5H, and INMT enzymes to convert tryptophan into various hydroxy tryptamines; and (D) a modified host organism expressing gene combinations with AADC, T5H, INMT, and IOMT enzymes to convert tryptophan into various methoxy tryptamines.

FIG. 10 depicts (A) a modified host organism which can generate various hydroxy tryptamines through bioconversion of serotonin provided exogenously or generated within the host organism; and (B) a modified host organism which can generate various methoxy tryptamines through bioconversion of melatonin provided exogenously or generated within the host organism.

FIG. 11 depicts (A) a scaffolded biosynthesis pathway of colocalized AADC, T5H-CPR fusion, IOMT, and NAT enzymes for conversion of tryptophan to melatonin; and (B) a modified host organism expressing the biosynthesis pathway from FIG. 11A to convert tryptophan to melatonin and related products.

FIG. 12 depicts (A) a scaffolded biosynthesis pathway of colocalized AADC, T4H-CPR fusion, PsiK, and PsiM enzymes for conversion of tryptophan to psilocybin related products; and (B) a modified host organism expressing the biosynthesis pathway from FIG. 12A to convert tryptophan to psilocybin and related products.

FIG. 13 depicts (A) example routes to halogenated, prenylated, and N-methylated alpha-methyl-tryptamine (AMT); and (B) a modified host organism expressing gene combinations to modify exogenously provided AMT to generate alpha-methylated-tryptamine variants.

FIG. 14 depicts (A) a heterologous tryptophan synthase (TrpS) route to combine synthetically modified indole with serine or threonine to generate indole modified tryptophan or indole modified beta-methyl tryptophan; and (B) a host organism expressing gene combinations to generate variants of indole modified tryptophan or indole modified beta-methyl tryptophan.

FIG. 15 depicts (A) the ATMT fusion enzyme converted tryptophan to beta-methyl tryptophan; and (B) a host organism expressing the ATMT fusion enzyme with gene combinations to generate beta-methyl tryptophan variants.

FIG. 16 depicts (A) the conversion of phosphorylated tryptamines to the corresponding hydroxy tryptamines by dephosphorylation; and (B) the oxidation of example hydroxy tryptamines which can catalyze polymerization.

FIG. 17 depicts HPLC chromatograms and UV-vis spectral matching of fermentation derived tryptamine via expression of the AADC enzyme.

FIG. 18 depicts HPLC chromatograms of fermentation derived methylated tryptamine via expression of the TrpM enzyme.

FIG. 19 depicts HPLC chromatograms of fermentation derived 4-OH tryptamine with improvements in yield via an optimal T4H-CPR fusion.

FIG. 20 depicts HPLC chromatograms of fermentation derived 5-OH-NMT via bioconversion of exogenous serotonin.

FIG. 21 depicts (A) a biosynthetic route to serotonin and 5-OH-NMT with a T5H enzyme or with a T5H-CPR fusion enzyme; and HPLC chromatograms of fermentation derived serotonin and 5-OH-NMT with improvements in yield via an optimal T5H-CPR fusion.

FIG. 22 depicts HPLC chromatograms of fermentation derived serotonin and melatonin.

FIG. 23 depicts HPLC chromatograms of fermentation derived 5-OH NMT and bufotenine.

FIG. 24 depicts HPLC chromatograms of fermentation derived psilocybin.

FIG. 25 depicts a synthetic route to methylate various tryptamines.

FIG. 26 depicts HPLC chromatograms and UV-vis spectral matching of fermentation derived DMT.

### DETAILED DESCRIPTION OF THE INVENTION

### Abbreviations and Definitions

To facilitate understanding of the invention, a number of terms and abbreviations as used herein are defined below as follows:

Conservative amino acid substitutions: As used herein, when referring to mutations in a protein, “conservative amino acid substitutions” are those in which at least one amino acid of the polypeptide encoded by the nucleic acid sequence is substituted with another amino acid having similar characteristics. Examples of conservative amino acid substitutions are ser for ala, thr, or cys; lys for arg; gln for asn, his, or lys; his for asn; glu for asp or lys; asn for his or gln; asp for glu; pro for gly; leu for ile, phe, met, or val; val for ile or leu; ile for leu, met, or val; arg for lys; met for phe; tyr for phe or trp; thr for ser; trp for tyr; and phe for tyr.

Functional variant: The term “functional variant,” as used herein, refers to a recombinant enzyme such as an INMTenzyme that comprises a nucleotide and/or amino acid sequence that is altered by one or more nucleotides and/or amino acids compared to the nucleotide and/or amino acid sequences of the parent protein and that is still capable of performing an enzymatic function (e.g., synthesis of DMT) of the parent enzyme. In other words, the modifications in the amino acid and/or nucleotide sequence of the parent enzyme may cause desirable changes in reaction parameters without altering fundamental enzymatic function encoded by the nucleotide sequence or containing the amino acid sequence. The functional variant may have conservative change including nucleotide and amino acid substitutions, additions and deletions. These modifications can be introduced by standard techniques known in the art, such as site-directed mutagenesis and random PCR-mediated mutagenesis, and may comprise natural as well as non-natural nucleotides and amino acids. Also envisioned is the use of amino acid analogs, e.g. amino acids not DNA or RNA encoded in biological systems, and labels such as fluorescent dyes, radioactive elements, electron dense agents, or any other protein modification, now known or later discovered.

Recombinant nucleic acid and recombinant protein: As used herein, a recombinant nucleic acid or protein is a nucleic acid or protein produced by recombinant DNA technology, e.g., as described in Green and Sambrook (2012).

Polypeptide, protein, and peptide: The terms “polypeptide,” “protein,” and “peptide” are used herein interchangeably to refer to amino acid chains in which the amino acid residues are linked by peptide bonds or modified peptide bonds. The amino acid chains can be of any length of greater than two amino acids. Unless otherwise specified, the terms “polypeptide,” “protein,” and “peptide” also encompass various modified forms thereof. Such modified forms may be naturally occurring modified forms or chemically modified forms. Examples of modified forms include, but are not limited to, glycosylated forms, phosphorylated forms, myristoylated forms, palmitoylated forms, ribosylated forms, acetylated forms, and the like. Modifications also include intra-molecular crosslinking and covalent attachment of various moieties such as lipids, flavin, biotin, polyethylene glycol or derivatives thereof, and the like. In addition, modifications may also include protein cyclization, branching of the amino acid chain, and cross-linking of the protein. Further, amino acids other than the conventional twenty amino acids encoded by genes may also be included in a polypeptide.

The term “protein” or “polypeptide” may also encompass a “purified” polypeptide that is substantially separated from other polypeptides in a cell or organism in which the polypeptide naturally occurs (e.g., 30%, 40%, 50%, 60%, 70%, 80%, 90%, 95%, 96%, 97%, 98%, 99%, 100% free of contaminants).

Primer, probe and oligonucleotide: The terms “primer,” “probe,” and “oligonucleotide” may be used herein interchangeably to refer to a relatively short nucleic acid fragment or sequence. They can be DNA, RNA, or a hybrid thereof, or chemically modified analogs or derivatives thereof. Typically, they are single-stranded. However, they can also be double-stranded having two complementing strands that can be separated apart by denaturation. In certain aspects, they are of a length of from about 8 nucleotides to about 200 nucleotides. In other aspects, they are from about 12 nucleotides to about 100 nucleotides. In additional aspects, they are about 18 to about 50 nucleotides. They can be labeled with detectable markers or modified in any conventional manners for various molecular biological applications.

Vector: As used herein, the term “vector” refers to a nucleic acid molecule capable of transporting another nucleic acid to which it has been linked. One type of vector is an episome, i.e., a nucleic acid capable of extra-chromosomal replication. Various vectors are those capable of autonomous replication and/expression of nucleic acids to which they are linked. Vectors capable of directing the expression of genes to which they are operatively linked are referred to herein as “expression vectors.”

Linker: The term “linker” refers to a short amino acid sequence that separates multiple domains of a polypeptide. In some embodiments, the linker prohibits energetically or structurally unfavorable interactions between the discrete domains.

Codon optimized: As used herein, a recombinant gene is “codon optimized” when its nucleotide sequence is modified to accommodate codon bias of the host organism to improve gene expression and increase translational efficiency of the gene.

Expression cassette: As used herein, an “expression cassette” is a nucleic acid that comprises a gene and a regulatory sequence operatively coupled to the gene such that the promoter drives the expression of the gene in a cell. An example is a gene for an enzyme with a promoter functional in yeast, where the promoter is situated such that the promoter drives the expression of the enzyme in a yeast cell.

Compounds and abbreviations in use of or contained within systems and methods herein are provided in Table 1.

TABLE 1

Compounds
tryptamine
L-tryptophan
L-methionine
bufotenin
4-hydroxy-tryptamine
norbaeocystin
norpsilocin
baeocystin
psilocybin
psilocin
aeruginascin
NMT = N-methyltryptamine
DMT = N,N-dimethyltryptamine
TMT = N,N,N-trimethyltryptamine
NMTP = N-methyltryptophan or L-Abrine
DMTP = N,N-dimethyltryptophan
TMTP = N,N,N-trimethyltryptophan or Hypaphorine or Lenticin
5-HTP = 5-hydroxytryptophan
SAMe = S-Adenosyl-L-methionine
SAH = S-Adenosyl-L-homocysteine
DMC = dimethylcarbonate
DMAPP = dimethylallyl diphosphate
DMSO = dimethyl sulfoxide
5-HT = 5-hydroxytryptamine or Serotonin
NAS = N-acetylserotonin or Normelatonin
NA-MeO-T = N-acetyl-5-methoxy-tryptamine or Melatonin
5-MT = 5-methoxy-tryptamine or Mexamine
5-MeO-NMT = 5-methoxy-N-methytryptamine
5-MeO-DMT = 5-methoxy-N,N-Dimethyltryptamine
5-MeO-TMT = 5-methoxy-N,N,N-trimethyltryptamine
5-HO-NMT = 5-hydroxy-methyltryptamine
5-HO-DMT = 5-hydroxy-dimethyltryptamine or Bufotenine
5-HO-TMT = 5-hydroxy-trimethyltryptamine or Bufotenidine
NMT = N-methyltryptamine
DMT = N,N-dimethyltryptamine
TMT = N,N,N-trimethyltryptamine
NMTP = N-methyltryptophan or L-abrine
DMTP = N,N-dimethyltryptophan
TMTP = N,N,N-trimethyltryptophan or Hypaphorine or Lenticin
5-HO-NMTP = 5-hydroxy-methyltryptophan
5-HO-DMTP = 5-hydroxy-dimethyltryptophan
5-HO-TMTP = 5-hydroxy-methyltryptophan
5-MeO-NMTP = 5-methoxy-methyltryptophan
5-MeO-DMTP = 5-methoxy-dimethyltryptophan
5-MeO-TMTP = 5-methoxy-methyltryptophan
BH4 = Tetrahydrobiopterin
BH2 = Dihydrobiopterin
NADPH = Reduced nicotinamide adenine dinucleotide phosphate
NADP+ = Nicotinamide adenine dinucleotide phosphate
Acetyl-CoA = Acetyl coenzyme A
β-Methyltryptophan (β-mTrp)
N-acetyl-4-hydroxy-tryptamine
N-acetyl-psilocybin
N-acetyl-psilocin

Enzymes and regulatory proteins, and abbreviations, in use of or contained within systems and methods herein are provided in Table 2.

TABLE 2

Enzymes and Regulatory Proteins
INMT = Indolethylamine-N-methyltransferase; tryptamine
N-methyltransferase
IOMT = indole-O-methyltransferase; hydroxytryptamine
O-methyltransferase
CaffOMT = caffeic acid-O-methyltransferase
T5H = tryptamine 5′ hydroxylase
TrpM = tryptophan N-methyltransferase
PsiM = psilocybin synthase
AADC = Aromatic amino acid decarboxylase; tryptophan decarboxylase
TPH = tryptophan hydroxylase
T4H = tryptamine 4′ hydroxylase
T4H-CPR, T5H-CPR = chimeras with cytochrome p450 reductase
NAT = N-acetyltransferase
DAC = deacetylase
BH4syn = Tetrahydrobiopterin synthesis
BH4reg = Tetrahydrobiopterin regeneration
PsiK = hydroxy tryptamine kinase
TrpS = tryptophan synthase
TMO = toluene monooxygenase
ATMT = aminotransferase/methyltransferase fusion
oxidase = multi-copper oxidase
DMATS = dimethylallyltryptophan synthase
IDI1 = isopentenyl-diphosphate isomerase
TrpHalo = tryptophan halogenase
T5H-IOMT fusion polypeptide
AOQS = aspartate oxidase/quinolinic acid synthase fusion
TAT2 = tryptophan importer
MUP1 = methionine importer
SAM3 = SAMe importer
FEX1 = fluoride exporter

The present invention is directed to biosynthetic production of molecules that are analogs of indoles, tryptophans, and tryptamines, which can also serve as precursors to larger tryptamine alkaloids, such as tryptamines and tryptophans modified by hydroxylation, halogenation, methylation, phosphorylation, prenylation, and halogenation in recombinant organisms.

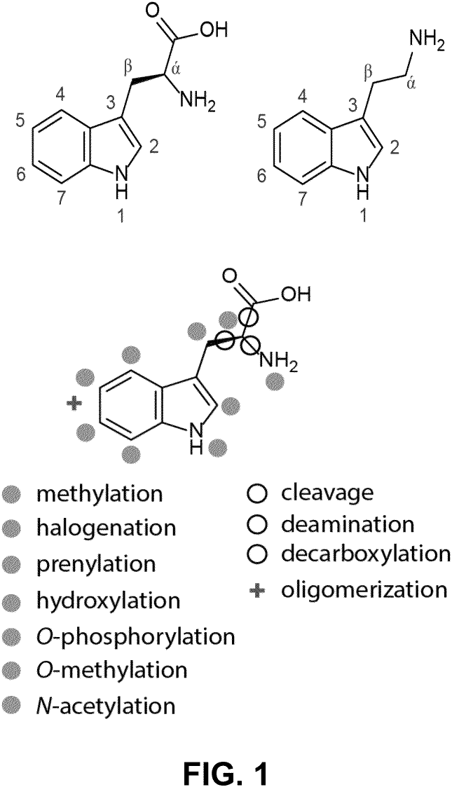

FIG. 1 shows the chemical structures of tryptophan (top left) and tryptamine (top right), along with enzyme modifications at specific reaction sites of the tryptophan molecule. Tryptophan is the precursor to a wide array of complex natural products. The electron-rich indole of tryptophan is a weak base. These properties allow for enhanced reactivity as a substrate for numerous enzymes that perform but are not limited to the following activities: methylation, halogenation, prenylation, hydroxylation, isonitrile synthesis, nitration, O-phosphorylation, O-methylation, O-acetylation, N-acetylation, glycosylation, sulfation, cleavage, deamination, decarboxylation, and oligomerization of the molecule. This diverse array of indole intermediates provides a way to tune psychedelic effects. For example, 5-MeO-DMT is reported to be more potent than DMT in neural rodent studies (Lima da Cruz, Rafael Vitor, et al.).

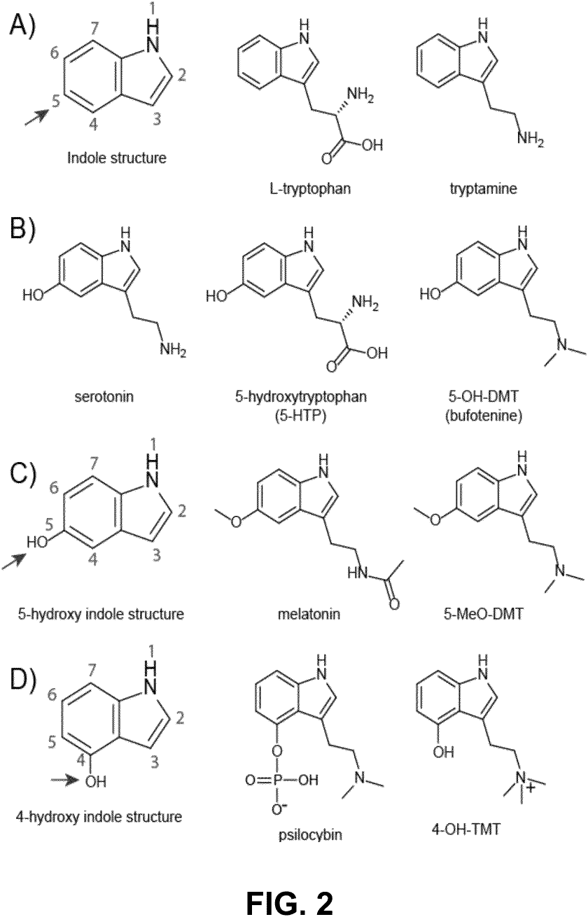

FIG. 2 shows examples of various substituted indole compounds in the tryptamine and tryptophan pathways utilized in the present invention. Panel A depicts the indole ring structure with positional numbering, and tryptophan and tryptamine. Examples of 5-hydroxy modified tryptophan and tryptamine compounds are shown in Panel B; Panel C shows examples of modified 5-hydroxy tryptamines. Additionally, Panel D shows examples of modified 4-hydroxy tryptamines.

By engineering various enzymes and regulatory proteins into a microorganism, tryptophan, tryptamine and other substituted indoles can be modified into a large array of useful compounds, which can be harvested from cultures of the microorganisms.

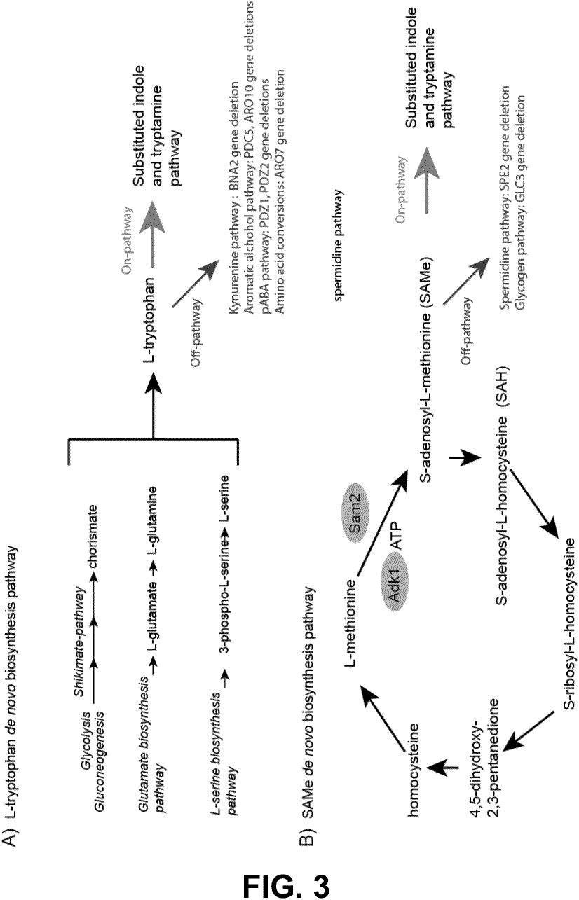

As depicted in FIG. 3, the de novo biosynthesis pathway of L-tryptophan and SAMe are utilized as directing molecules in the systems and methods herein. The directing molecules lead to target molecules of the substituted indoles and tryptamine pathways, when on-pathway. In the systems and methods herein, glycolysis leads to chorismate via the shikimate pathway; glutamate biosynthesis pathway leads to L-glutamine via L-glutamate; and L-serine biosynthesis pathway leads to L-serine via 3-phospho-L-serine (i.e., dephosphorylation). Chorismate, glutamine, and L-serine are combined to form L-tryptophan as a directing molecule to be steered on-pathway for yielding substituted indoles and tryptamine pathways. In the systems and methods herein, L-methionine is a direct precursor leading to SAMe, when combined with ATP in the presence of Sam2 and Adk1 enzymes. A conversion cycle for yielding SAMe as a directing molecule also involves the formation of S-adenyl-L-homocysteine; S-ribosyl-L-homocysteine; 4-5-dihydroxy-2,3-pentanedione; and homocysteine.

### Nucleic Acids

Thus, in some embodiments, provided is a non-naturally occurring nucleic acid comprising a sequence encoding an enzyme or regulatory protein in tryptamine metabolism, where the enzyme or regulatory protein is an N-methyltransferase (INMT, PsiM, TrpM), a tryptophan decarboxylase (AADC), a tryptophan hydroxylase (TPH), a tryptamine 4′ hydroxylase (T4H), a tryptamine 5′ hydroxylase (T5H), a truncated cytochrome p450 reductase (T4H-CPR, T5H-CPR), an hydroxytryptamine O-methyltransferase (IOMT or CaffOMT), an N-acetyltransferase (NAT), a deacetylase (DAC), a hydroxyl tryptamine kinase (PsiK), a tryptophan synthase (TrpS), a toluene monooxygenase (TMO), an aminotransferase/methyltransferase fusion (ATMT), a phosphatase, an oxidase, a dimethylallyltryptophan synthase (DMAT or DMATS), an isopentenyl-diphosphate isomerase (IDI1), a tryptophan halogenase (TrpHalo), an aspartate oxidase/quinolinic acid synthase fusion (AOQS), a tryptophan importer (TAT2), a methionine importer (MUP1), or a SAMe importer (SAM3).

These enzymes and regulatory proteins are further characterized as follows.

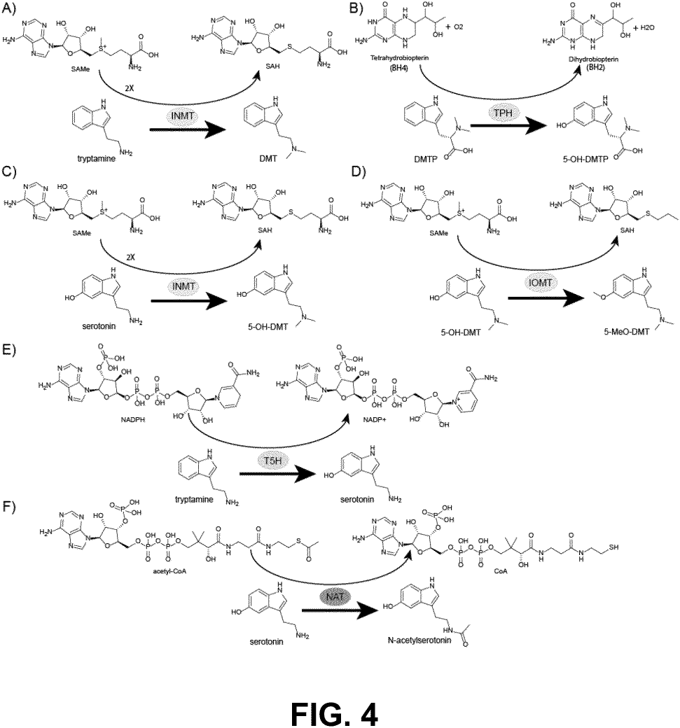

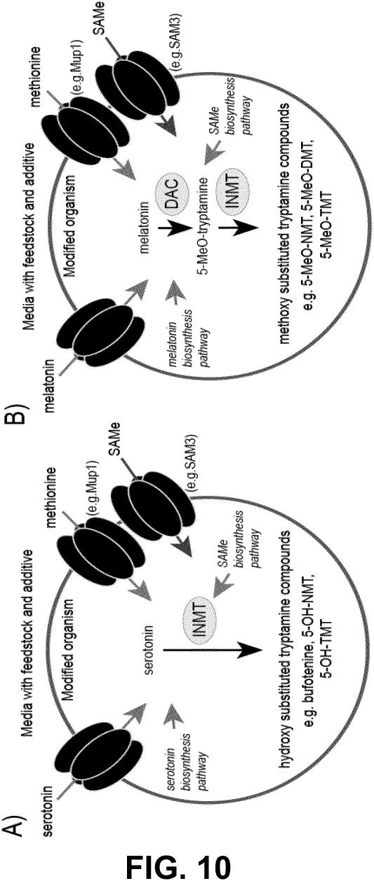

Indolethylamine N-methyltransferase (INMT) catalyzes the alkylation (i.e., adding a methyl (CH₃) group) of the primary amine on a tryptamine substrate. The methylation reaction uses up the methyl donor cofactor, SAMe (see FIG. 4, Panels A and C). As an example of INMT activity, INMT can act on serotonin to create 5-OH-DMT (bufotenine) or tryptamine to create DMT (FIG. 4, Panels A and C; FIG. 10, Panel A).

Indole-O-methyltransferase (IOMTor CaffOMT) catalyzes the alkylation of the primary amine on the 5-hydroxy moiety on an indole ring. The methylation reaction uses up the methyl donor cofactor, SAMe (FIG. 4, Panel D). As an example of IOMT activity, IOMT can act on bufotenine (5-OH-DMT) to create 5-MeO-DMT, or N-acetylserotonin to create melatonin (FIG. 4, Panel D).

Tryptamine 5′ hydroxylase (T5H) is a p450 tryptamine hydroxylase which prefers hydroxylation at the 5′ position of the indole ring, such as generating serotonin from tryptamine (FIG. 4, Panel E), in conjunction with the cofactors NAD(P)H, FMN, and FAD+. P450s such as the T5Hs are generally membrane-associated, with the N-termini imparting an effect on the efficiency of the p450 enzymatic function, including a p450′s interaction with an associated CPR, which assists with electron transfer.

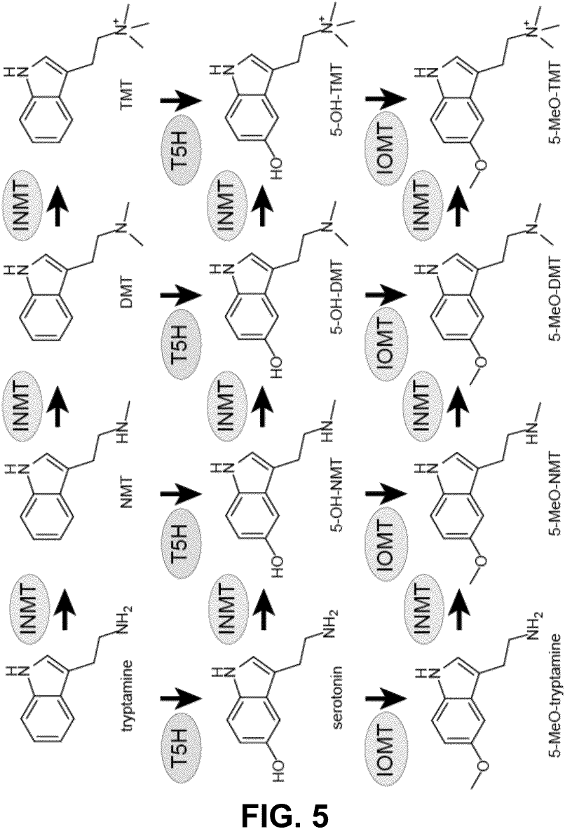

FIG. 5 shows a matrix of various compounds that can be made with INMT, IOMT and T5H.

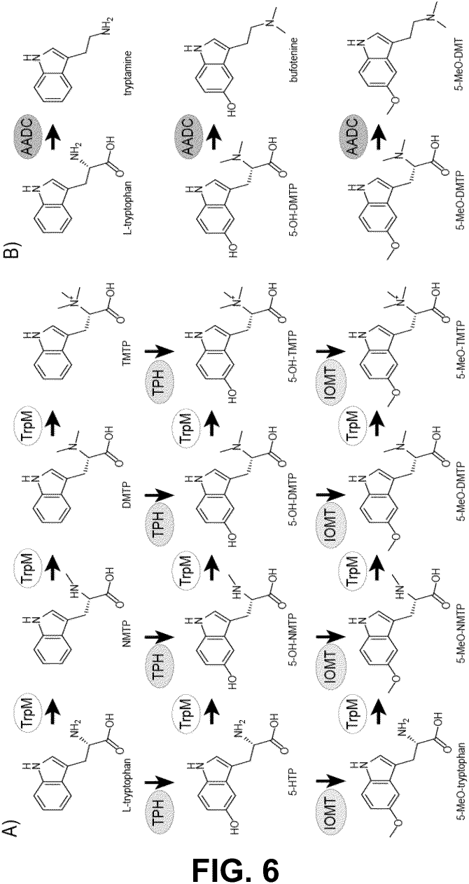

Tryptophan methyltransferase (TrpM) catalyzes the alkylation of the primary amine of L-tryptophan to produce N-methyltryptophan (NMTP, also called L-abrine), the mono-methylated product; N,N-dimethyltryptophan (DMTP), the di-methylated product; and N,N,N-trimethyltryptophan (TMTP), the tri-methylated product. See FIG. 6, Panel A.

Psilocybin synthase (PsiM) is an N-methyltransferase that prefers a substituted tryptamine, such as the phosphorylated tryptamine, norbaeocystin. Novel chimeric PsiMs, were generated to remove potentially deleterious regulatory regions of the enzymes by swapping PsiM domains with the related small rRNA methyltransferases from Ascomycota, the phylum of S. cerevisiae.

Aromatic amino acid decarboxylase or tryptophan decarboxylase (AADC) catalyzes the decarboxylation of an aliphatic carboxylic acid (i.e., releases carbon dioxide) from compounds such as L-tryptophan to create tryptamine, 5-HTP to create serotonin; 5-OH-DMTP to create bufotenine; and 5-MeO-DMTP to create 5-MeO-DMT, as depicted in FIG. 6, Panel B.

Tryptophan hydroxylase (TPH), adds a hydroxy group to the 5-carbon of L-tryptophan. The L-tryptophan hydroxylase can catalyze the OH addition to the 5-carbon with the cofactor BH4 and oxygen (Biotechnol J. 2016 May;11(5):717-24) (FIG. 6, Panel A). BH4 is synthesized and regenerated in the cell with the BH4syn and BH4reg heterologous enzymes described herein. The BH4syn genes are enzymes that function as a GTP hydroxylase I, a 6-pyruvoyl-tetrahydropterin synthase, and a sepiapterin reductase to generate the BH4 cofactor necessary for TPH enzyme function. The BH4reg genes are enzymes that function as a 4a-hydroxytetrahydropterin dehydratase and a 6-pyruvoyl-tetrahydropterin synthase to regenerate the BH4 cofactor after conversion to HTHB by the TPH enzyme. As an example of TPH activity, TPH can act on L-tryptophan to generate 5-hydroxy-L-tryptophan (5-HTP), and 5-HTP can then be acted on by an AADC to generate serotonin.

Tryptamine 4′ hydroxylase (T4H) is a p450 tryptamine hydroxylase which prefers hydroxylation at the 4′ position of the indole ring, in conjunction with the cofactors NAD(P)H, FMN, and FAD+. When derived from psychedelic mushrooms, these are also called PsiH. The T4H enzyme can convert tryptamine to 4-OH-tryptamine, which is a part of the psilocybin pathway. P450s such as the T4Hs are generally membrane-associated, with the N-termini imparting an effect on the efficiency of the p450 enzymatic function, including a p450′s interaction with an associated CPR, which assists with electron transfer.

From psychedelic a mushroom derived PsiH and CPR, we generated chimeric p450s and CPRs to better match a heterologous host (SEQ ID NO:162, 179-180, and 451, 468-469), where the N termini of a yeast p450 and CPR replaced the N terminus. Due to the enhancing action of CPRs on p450 enzymatic activity, we determined an optimal fusion between T4H and T4H CPR, where the T4H_CPR listed are truncated at the N termini and replaced with a linker region. In some embodiments, the T4H nucleic acids have, at the 3′ end, an optimized nucleic acid encoding a T4H_CPR, e.g., having SEQ ID NOs:171-180, joining the sequences together to form a fusion polypeptide, e.g., having the amino acid sequence of SEQ ID NOs:460-469 fused at the C terminus of the enzyme polypeptide, generating recombinant T4H-CPR fusion polypeptides.

Similar to the T4H CPR fusions, we generated T5H CPR fusions to enhance the hydroxylation activity. In those embodiments, the T5H nucleic acids have, at the 3′ end, an optimized nucleic acid encoding a T5H-CPR, e.g., having SEQ ID NOs:181-192, joining the sequences together to form a fusion polypeptide, e.g., having the amino acid sequence of SEQ ID NOs:470-481 fused at the C terminus of the enzyme polypeptide, generating recombinant T5H-CPR fusion polypeptides.

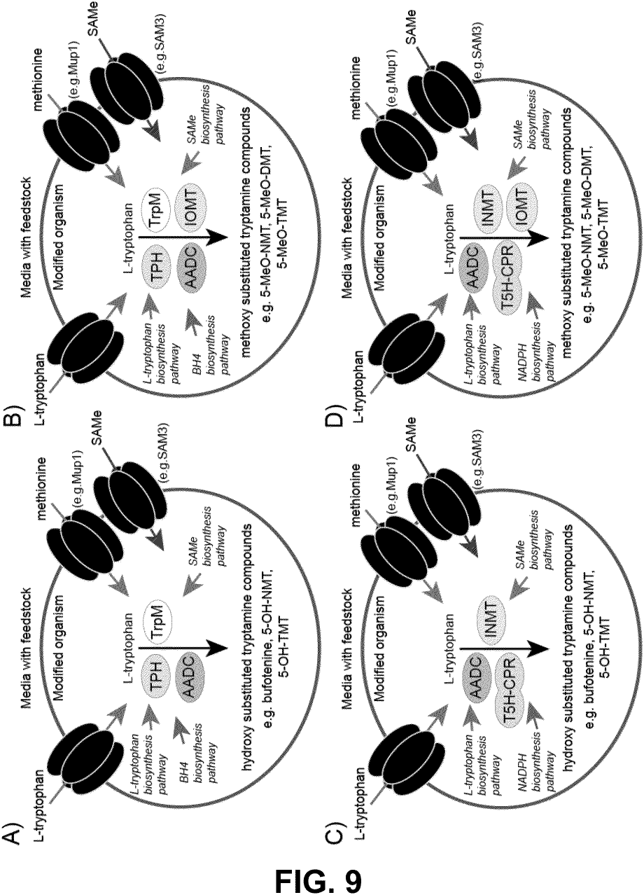

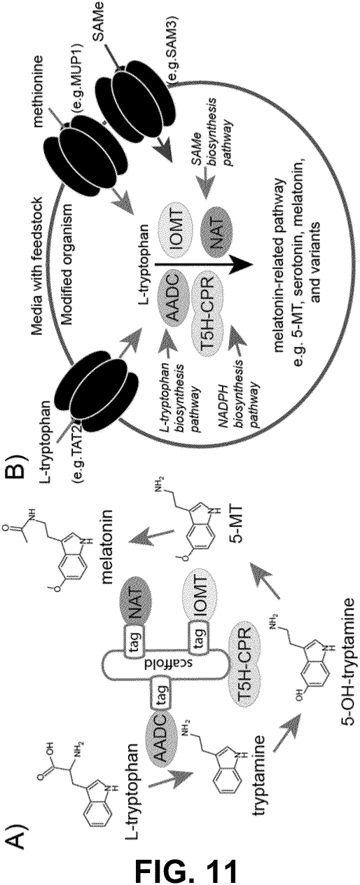

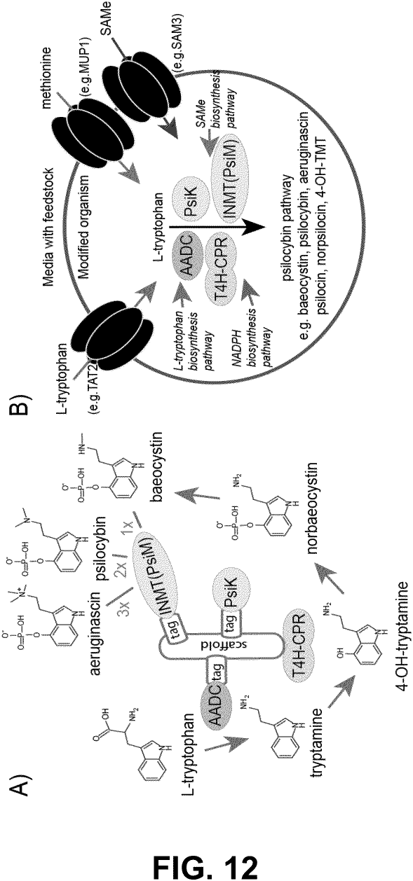

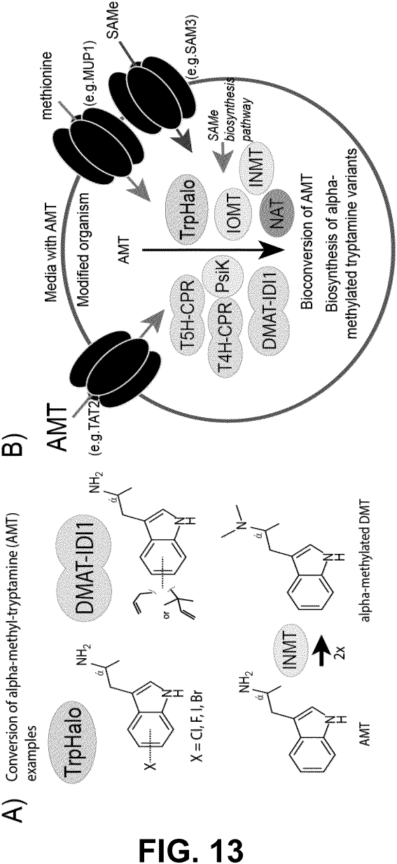

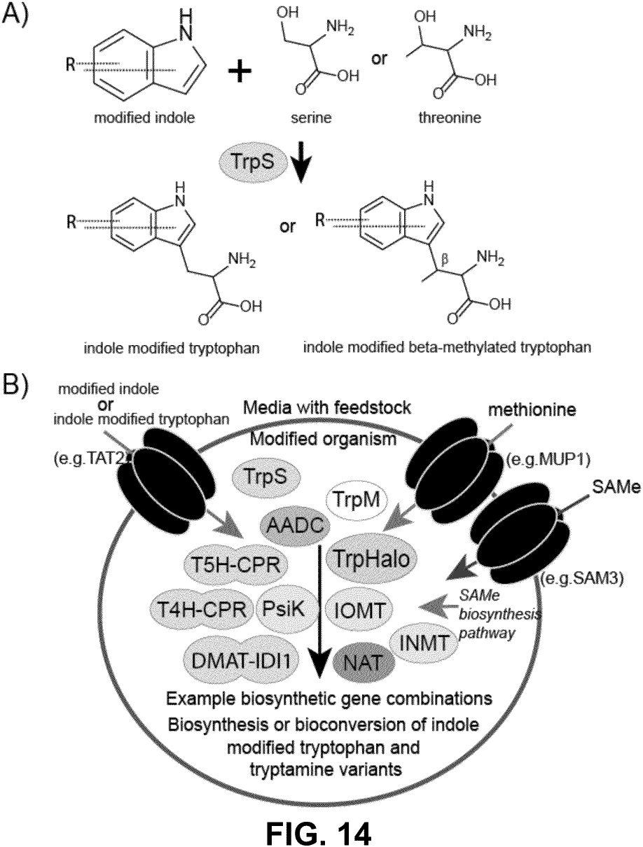

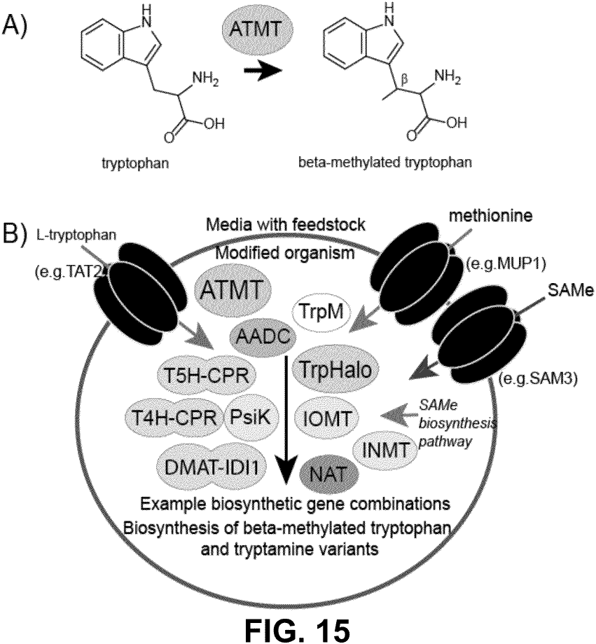

Examples of the utilization of the T4H-CPR and T5H-CPR in recombinant cells are shown in FIG. 9, Panels C and D; FIGS. 11 and 12; FIG. 13, Panel B; FIG. 14, Panel B; and FIG. 15, Panel B.

Localizing O-methyltransferase activity to hydroxylation can be beneficial for generating methoxytryptamines, such as 5-MT, 5-MeO-DMT, and melatonin. In some embodiments, the T5H nucleic acids have, at the 3′ end, an optimized nucleic acid encoding an IOMT e.g., having SEQ ID NOs:99-130, joining the sequences together to form a fusion polypeptide, e.g., having the amino acid sequence of SEQ ID NOs:388-419 fused at the C terminus of the enzyme polypeptide, generating recombinant T5H-IOMT fusion polypeptides.

In all of the fusions described herein, the N-terminal coding sequence has any STOP codon removed, if present, before fusion to a C-terminal coding sequence. If the N-terminal coding sequence does not have a START (ATG) codon, a START codon is added.

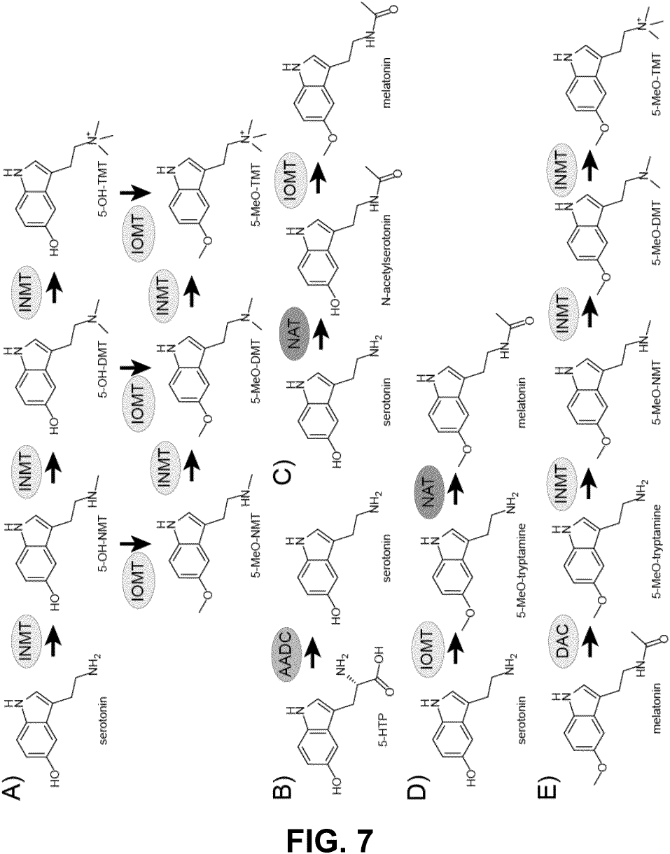

N-acetyltransferase (NAT) adds an acetyl group from acetyl-CoA to the terminal amino group of e.g., a tryptamine such as serotonin (FIG. 4, Panel F; FIG. 7, Panels C and D). As an example of NAT activity, NAT can act on serotonin to generate N-acetylserotonin, which in turn can be acted on by an IOMT to generate melatonin (FIG. 11).

Deacetylase (DAC) removes an acetyl group from the terminal amino group of a tryptamine such as melatonin. As an example of DAC activity, DAC can act on melatonin to create 5-MeO-tryptamine, which in turn can be acted on by an INMT to generate 5-MeO-DMT (FIG. 10, Panel B).

Hydroxy tryptamine kinase (PsiK) phosphorylates a hydroxy-indole, in conjunction with ATP. For example, PsiK can act on 4-OH tryptamine to generate norbaeocystin as part of the psilocybin pathway. PsiKs are found in certain mushrooms and parasitic fungi. For psychedelic mushroom derived PsiKs, we generated chimeric PsiKs based on yeast choline kinase to better match a heterologous host.

Non-natural tryptamine analogs can be created with the addition of a synthetic precursor to the fermentation of a recombinant host expressing enzymes capable of utilizing the substrate. For example, the addition of an alpha-methylated amino acid such as alpha-methyl tryptophan to a fermentation where an organism expresses a an indole-N-methyltransferase (INMT) leads to the generation of alpha-methylated DMT (e.g., FIG. 13).

For certain indole ring modifications, such as non-natural indoles, bacterial tryptophan synthases (TrpS) can be used to combine an indole with L-serine or L-threonine to create variants of tryptophan and beta-methyl tryptophan, respectively (FIG. 14, Panel A). While previous groups have made use of the flexibility of versions of bacterial tryptophan synthases to generate exotic tryptamines (De novo Biosynthesis of “Non-Natural” Thaxtomin Phytotoxins. Angew Chem Int Ed Engl. 2018 Jun 4;57(23):6830-6833), efficient bioproduction is limited by the toxic nature of indole. In one embodiment, TrpS is expressed as a modified secreted fusion polypeptide version of the Salmonella tryptophan synthase that is able to combine indole or a modified indole with L-serine or L-threonine in the extracellular space, allowing indole conversion away from the cell host. In some embodiments, a multidrug efflux exporter such as mdtEF (accessions: P37636, P37637) can be coexpressed with TrpS with exogenous indole, to enable the host cell to export indole and continue bioproduction of tryptophan and tryptamine analogs.

Alternatively to T4H, T5H, and TPH enzymes, hydroxylation of the indole ring of tryptamines and related indole-like compounds can be carried out by complexes known as toluenemonooxygenases (TMO) typically found in bacteria within the genus Pseudomonas. The polypeptides that form this complex can be expressed in a modified host as an alternative to P450-based hydroxylation for compounds such as psilocybin and aeruginascin, whose biosynthetic pathway involves 4′OH hydroxylation. Other non-P450 monooxygenases from genuses of Pseudomonas and Burkeholderia can be optimized and expressed in a modified host for hydroxylation of different indole positions, such as the 3′ carbon of the indole ring. TMO complexes are made up of several subunits. For efficient expression of TMOs in a recombinant heterologous host, we generated fusion polypeptide pairs of the four core subunits.

Beta-methylated tryptamine analogs are created by combined expression of a recombinant aminotransferase-methyltransferase (ATMT) fusion polypeptide and an aromatic amino acid decarboxylase (AADC) (FIG. 15). In nature, organisms which produce beta-methyl tryptophan typically express the aminotransferase (AT) and the methyltransferase (MT) as separate genes. Recombinant ATMT genes herein encode both domains as a single polypeptide. Combinatorial expression of ATMTs and other tryptamine modifying genes can be used to create compounds such as beta-methylated DMT and beta-methylated psilocybin.

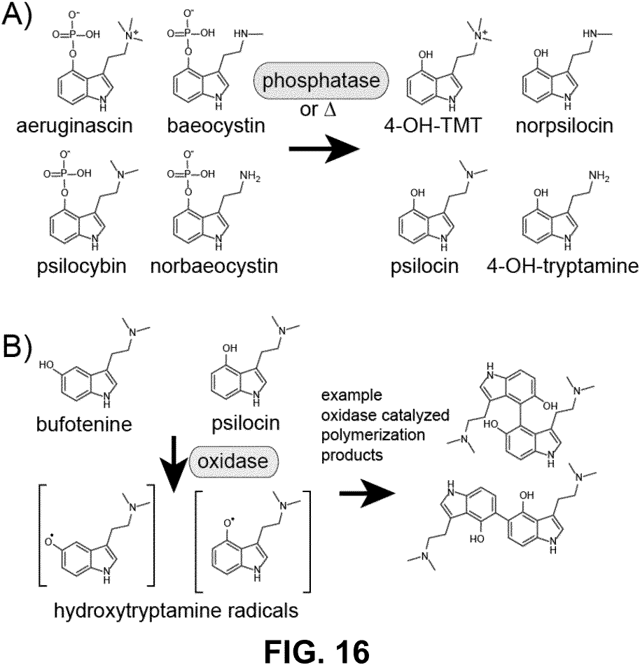

In some embodiments, recombinant phosphatases and oxidases are used to generate hydroxylated tryptamine dimers such as one psilocin or bufotenine molecule conjugated to another psilocin or bufotenine molecule (FIG. 16). When certain psychedelic mushrooms which contain compounds such as psilocybin are damaged and cellular compartments compromised, phosphatases and oxidases, such as laccases or laccase-like multi-copper oxidases, can then come in contact with tryptamine substrate to dephosphorylate and catalyze hydroxy tryptamine polymerization. Similar polymerization which leads to ‘blueing’ can occur when psilocybin comes into contact with mitochondria. (Levine, Walter (5), In some embodiments, the phosphatase is a recombinant alkaline phosphatase, which dephosphorylates phosphorylated tryptamines and tryptophans (FIG. 16, Panel A), such as psilocybin to psilocin. In some embodiments, the oxidase is a non-laccase member of the multi-copper oxidase superfamily, which creates hydroxy tryptamine radicals which catalyze polymerization (FIG. 16, Panel B). This dimer example and oligomerization of hydroxylated tryptamines can generate a blue color, lending the effect to colorimetric readout for compound production. Dimer variants and other oligomerized tryptamines can be separated from each other through chromatographic methods for purification. Efficient heterologous expression of certain oxidases such as laccases presents several challenges, such as N and C termini processing which may fail in a heterologous host. In some embodiments, to improve heterologous oxidase expression to biosynthetically produce tryptamine dimers and oligomers, we engineered chimeric oxidase yeast oxidase. Example includes SEQ ID NO:274,563 In one embodiment, the oxidases are also coexpressed with the yeast t-SNARE, SSO2, to improve protein expression, processing, and secretion for active enzyme SEQ ID NO:170,459.

Dimethylallyl tryptophan synthase (DMATS or DMAT) generates prenylated tryptophans and tryptamines. DMATS is a prenyltransferase that prefers the dimethylallyl diphosphate (DMAPP) prenyl donor to prenylate tryptophan and tryptamine compounds.

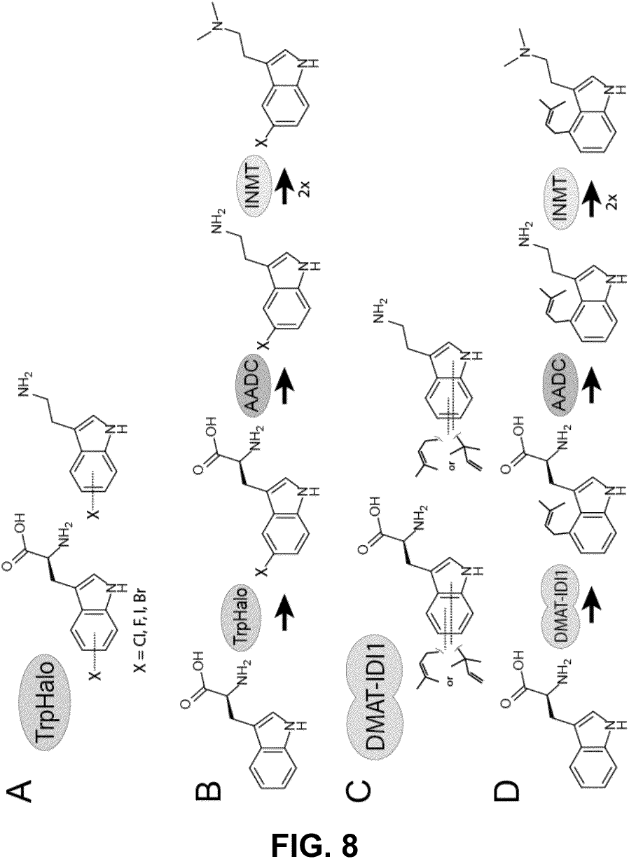

Localizing DMAPP generation to the DMATS enzyme can be beneficial for generating prenylated tryptophans, such as 7-dimethylallyltryptophan. In yeast, IDI1 is the enzyme which generates DMAPP as part of the mevalonate pathway. In some embodiments, the DMATS nucleic acids have, at the 3′ end, an optimized nucleic acid encoding IDI1 e.g., having SEQ ID NO:67, joining the sequences together to form a fusion polypeptide, e.g., having the amino acid sequence of SEQ ID NO:356 fused at the C terminus of the enzyme polypeptide, generating recombinant DMATS-IDI1 fusion polypeptides (FIG. 8, Panel C).

Tryptophan halogenase (TrpHalo) is a flavin-associated halogenase that adds fluorine (F), chlorine (Cl), bromine (Br), and/or iodine (I) to various indoles and biogenic amines (FIG. 8, Panel A). In some embodiments, TrpHalo nucleic acids have, at the 5′end, a nucleic acid encoding an vacuolar localization tag to localize TrpHalo to a yeast vacuole, where Cl ions are stored, e.g., having SEQ ID NOs:287-289, joining the sequences together to form a fusion polypeptide, e.g., having the amino acid sequence of SEQ ID NOs:576-578 fused at the N terminus of the enzyme polypeptide, generating recombinant fusion polypeptides.

In other embodiments, TrpHalo nucleic acids have, at the 5′end, a nucleic acid encoding a secretion tag with or without a 6xHIS tag for purification, e.g., having SEQ ID NO:1, joining the sequences together to form a fusion polypeptide, e.g., having the amino acid sequence of SEQ ID NO:290 fused at the N terminus of the enzyme polypeptide, generating recombinant fusion polypeptides. In one embodiment, TrpHalo is also coexpressed with the yeast fluoride exporter, Fex1, SEQ ID NO:66,355, to limit halide toxicity on the heterologous host.

To improve the yield of tryptophan and tryptamine variants discussed herein, modifying the heterologous host which expresses these genes and enzymes, in various combinatorial ways, to prevent tryptophan and tryptamine compound degradation is beneficial. Replacing the yeast pathway which degrades certain tryptamine and tryptophan compounds for de novo NAD+ production, which is an important source of cofactors for cell viability, with an alternative route to NAD+ production can preserve tryptophan as a precursor and increase product yields. In some embodiments, a new de novo pathway is expressed in a heterologous host, where the pathway is composed of a fusion protein containing the two enzymatic functions required to convert the amino acid aspartate into quinolinic acid (AOQS), SEQ ID NO: 26-27,315-316, which replaces the endogenous use of tryptophan for generating quinolinic acid in the pathway for NAD+.

In some embodiments, the nucleic acids have, at the 5′ end, a nucleic acid encoding codon optimized cofolding peptides to create a fusion protein, e.g., having SEQ ID NOs:256-269, joining the sequences together to form a fusion polypeptide, e.g., having the amino acid sequence of SEQ ID NOs:554-558 fused at the N terminus of the enzyme polypeptide, generating recombinant fusion polypeptides.

In some embodiments, the nucleic acids have, at the 5′ end, a nucleic acid encoding a secretion signal, creating a secreted protein, e.g., having SEQ ID NOs:282-286, joining the sequences together to form a fusion polypeptide, e.g., having the amino acid sequence of SEQ ID NOs:571-575 fused at the N terminus of the enzyme polypeptide, generating recombinant fusion polypeptides.

In some embodiments, the nucleic acids have, at the 5′ or 3′ end, an optimized nucleic acid encoding a localization scaffold composed of multiple domains where proteins tagged with affibodies can bind and colocalize together (for example, FIG. 11, Panel A; FIG. 12, Panel A), creating a protein scaffold fusion, e.g., having SEQ ID NO:281, joining the sequences together to form a fusion polypeptide, e.g., having the amino acid sequence of SEQ ID NO:570 fused at the N or C terminus of the enzyme polypeptide, generating recombinant fusion polypeptides.

In some embodiments, the nucleic acids have, at the 5′ or 3′ end, an optimized nucleic acid encoding an affibody tag that can bind one of the domains of the localization scaffold, thereby colocalizing multiple enzymes and creating protein scaffold fusion, e.g., having SEQ ID NOs:259-264, joining the sequences together to form a fusion polypeptide, e.g., having the amino acid sequence of SEQ ID NOs:548-553 fused at the N or C terminus of the enzyme polypeptide, generating recombinant fusion polypeptides.

The initial substrates for DMTP, DMT, and related compound production are L-tryptophan and S-Adenosyl-L-methionine (SAMe). The initial substrate can be produced endogenously in a recombinant host as described and/or provided exogenously to a fermentation involving a recombinant host, whereby the host uptakes the starting substrates to feed into the biosynthetic pathway for indoles and tryptamines. The recombinant hosts herein described that are expressing all, one, or multiple combinations of the engineered INMT, AADC, TPH, T4H, T5H, T4H-CPR, T5H-CPR, IOMT, NAT, DAC, PsiK, TrpS, TMO, ATMT, DMATS, IDI1, and TrpHalo genes can produce tryptamine, NMTP, DMTP, TMTP, NMT, DMT, TMT, psilocybin, bufotenine, 5-MeO DMT, 4-bromo-tryptamine, 4-dimethylallyl tryptamine, alpha-methylated DMTP, beta-methylated DMTP, melatonin, etc.

As depicted in FIGS. 4 and 9, the engineered INMT, IOMT and TrpM and INMT enzymes require a methyl donor in the form of SAMe to act on substrates in the biosynthetic pathway for substituted indoles and tryptamines such as DMTP, DMT, intermediates, and analogs. The methyltransferase activity of TrpM and INMT subsequently convert the methyl donor cofactor SAMe to SAH. Methylations can occur successively with multiple rounds of methyl donor usage. For instance, TrpM can methylate L-tryptophan to produce NMTP and continue to methylate NMTP to DMTP, and then TMTP (FIG. 6, Panel A). Similarly, an INMT can methylate tryptamine to produce NMT, and then continue to methylate NMT to DMT, and then TMT (FIG. 5).

The methylation occurs selectively at the primary amine of L-tryptophan and tryptamine in the presence of TrpM and INMT enzymes. The nitrogen in the heterocycle and hydroxyl group in the carboxylic acid of L-tryptophan are also sites of alkylation, as SAMe is a highly reactive methylating agent. The TrpM enzyme directs methylation such that di-methylation of the primary amine occurs. Using the traditional techniques of organic chemistry where robust methylating agents, such as methyl iodide, trimethyl sulfonium iodide, and dimethyl sulfate, are employed, a mixture of products is formed. The mixture of products may include: mono, di, and tri-methylation of the amine; O-methylation of the carboxylic acid (i.e., the methyl ester), and N-methylation of the indole ring. Separation of these products are tedious and reduces the yield of a desired product. Additionally, SAMe has a primary amine group which may readily undergo intramolecular methylation at the amine. The systems and methods herein in the recombinant host with TrpM and INMT enzymes maintain the structure of SAMe without methylation of the amine of the SAMe prior to methylating the amine of L-tryptophan and tryptamine.

Heterologous pathway enzymes that are expressed to produce substituted indole and tryptamine compounds such as DMTP and DMT use L-tryptophan as a directing molecule. Tryptophan production in cells is normally tightly regulated. Tryptophan accumulation in a recombinant host is increased by: (a) overexpressing feedback-resistant versions of the endogenous tryptophan-producing enzymes; (b) knocking out off-pathway tryptophan-consuming genes and enzymes; and (c) overexpressing a recombinant L-tryptophan transporter. This allows for exogenous tryptophan to be fed to the cells and transported in the recombinant host. These modifications, genes, and methods are disclosed in U.S. Pat. Publication 2021/0147888, incorporated by reference.

On-pathway genes and enzymes can be overexpressed for L-tryptophan accumulation. The immediate precursors for L-tryptophan include chorismate, L-serine, and L-glutamine. To increase the on-pathway flux to L-tryptophan and the substituted indole and tryptamine pathway, off-pathway genes which consume L-tryptophan are deleted. The genes that encode the enzymes, Pdc5 and Aro10 are deleted to reduce pathway flux through the pathways that produce aromatic alcohols. The gene encoding the Aro7 enzyme is deleted to reduce production of tyrosine and phenylalanine from L-tryptophan. The genes that encode the enzymes Pdz1 and Pdz2 are also deleted to reduce pathway flux through the pABA production pathway. The gene encoding the enzyme Bna2 is deleted to reduce consumption of L-tryptophan by the kynurenine pathway.

In some embodiments, a recombinant host is modified to increase the accumulation of the methyl donor, SAMe, which is used by the recombinant TrpM and INMT enzymes to methylate indole and tryptamine molecules, such as L-tryptophan and NMT. SAMe accumulation in the recombinant host cell is increased by: (a) overexpressing enzymes to promote conversion of L-methionine to SAMe; (b) deleting off-pathway genes which encode for enzymes that deplete SAMe for unwanted side products; and (c) overexpressing a permease. This enables exogenous L-methionine to be fed to and transported into the cells.

The TrpM and INMT methyltransferase reactions consume one equivalent of adenosine triphosphate (ATP) and of SAMe. SAMe is a robust methyl donor synthesized from methionine and ATP via the L-methionine adenosyltransferase enzyme, Sam2. In various embodiments, Sam2 is overexpressed in a recombinant host to increase the conversion of L-methionine to SAMe. In other embodiments, to support the increased pathway flux and generate more ATP, the adenylate kinase enzyme, Adk1, is overexpressed. In additional embodiments, to increase the uptake of exogenous L-methionine fed into the SAMe pathway, recombinant Mup1 is overexpressed, which is a methionine transporter. SAMe is a precursor molecule for spermidine production and glycogen biosynthesis. To keep SAMe levels high in the pathways of the recombinant host and decrease off-pathway usage of SAMe, the SPE2 gene can be deleted in the recombinant host, thereby blocking the conversion of SAMe to spermidine. Glycogen biosynthesis consumes ATP, which is required for the conversion of L-methionine to SAMe. The gene encoding the enzyme Glc3 can be deleted in the recombinant host, thereby reducing production of glycogen, maintaining higher levels of ATP in the host cell, and increasing on-pathway flux of SAMe for methyltransferase activity.

As depicted in FIG. 5, the engineered INMT, T5H, and IOMT enzymes act on tryptamine substrates to generate hydroxy and methoxy tryptamine analogs such as serotonin, bufotenine (5-OH-DMT) and 5-MeO-DMT. The initial substrates for this series of reactions includes compounds such as tryptamine and serotonin, which can be produced within a modified cell or added exogenously, in addition to L-tryptophan and S-Adenosyl-L-methionine (SAMe). The initial substrate can be produced endogenously in a recombinant host as described and/or provided exogenously to a fermentation involving a recombinant host, whereby the host uptakes the starting substrates to feed into the biosynthetic pathway for indoles and tryptamines.

As depicted in FIG. 3, de novo biosynthesis pathway of L-tryptophan and SAMe utilize L-tryptophan and SAMe as directing molecules in the systems and methods herein. The directing molecules lead to target molecules of substituted indoles and tryptamine pathways, when on-pathway. In the systems and methods herein, glycolysis leads to chorismate via the shikimate pathway; glutamate biosynthesis pathway leads to L-glutamine via L-glutamate; and L-serine biosynthesis pathway leads to L-serine via 3-phospho-L-serine (i.e., dephosphorylation). Chorismate, glutamine, and L-serine are combined to form L-tryptophan as a directing molecule to be steered on-pathway for yielding substituted indoles and tryptamine pathways. In the systems and methods herein, L-methionine is a direct precursor leading to SAMe, when combined with ATP in the presence of Sam2 and Adk1 enzymes. A conversion cycle for yielding SAMe as a directing molecule also involves the formation of S-adenyl-L-homocysteine; S-ribosyl-L-homocysteine; 4-5-dihydroxy-2,3-pentanedione; and homocysteine.

Heterologous pathway enzymes that are expressed to produce substituted indole and tryptamine compounds such as DMTP and DMT use L-tryptophan as a directing molecule. Tryptophan production in cells is normally tightly regulated. Tryptophan accumulation in a recombinant host is increased by: (a) overexpressing feedback-resistant versions of the endogenous tryptophan-producing enzymes; (b) knocking out off-pathway tryptophan-consuming genes and enzymes; and (c) overexpressing a recombinant L-tryptophan transporter. This allows for exogenous tryptophan to be fed to the cells and transported in the recombinant host. See also U.S. Pat. Publication 2021/0147888.

On-pathway genes and enzymes can be overexpressed for L-tryptophan accumulation. The immediate precursors for L-tryptophan include chorismate, L-serine, and L-glutamine. To increase the on-pathway flux to L-tryptophan and the substituted indole and tryptamine pathway, off-pathway genes which consume L-tryptophan may be deleted. In some embodiments, the genes that encode the enzymes Pdc5 and Aro10 are deleted to reduce pathway flux through the pathways that produce aromatic alcohols. In other embodiments, the gene encoding the Aro7 enzyme is deleted to reduce production of tyrosine and phenylalanine from L-tryptophan. In additional embodiments, the genes that encode the enzymes Pdz1 and Pdz2 are also deleted to reduce pathway flux through the pABA production pathway. In further embodiments, the gene encoding the enzyme Bna2 is deleted to reduce consumption of L-tryptophan by the kynurenine pathway.

In some embodiments, the nucleic acids described herewith encode a polypeptide or oligopeptide having an amino acid sequence that is naturally occurring. In other embodiments, the nucleic acids encode a polypeptide or oligopeptide having an amino acid sequence that is not naturally occurring. The encoded polypeptides or oligopeptides that are not naturally occurring can vary from a naturally occurring polypeptide or oligopeptide, or portion thereof, by a small amount (e.g., one conservative amino acid substitution or a histidine tag) or extensively (e.g., further comprising a fusion peptide, a substituted or added domain from another protein, a scaffold, etc.).

The nucleic acids can be derived from a naturally occurring gene from any source, e.g., any microorganism, protist, plant, or animal.

In some embodiments, the gene for the enzyme or regulatory protein is derived from a bacterium. It is envisioned that an enzyme or regulatory protein derived from any bacterium now known or later discovered can be utilized in the present invention. For example, the bacterium can be from phylum Abditibacteriota, including class Abditibacteria, including order Abditibacteriales; phylum Abyssubacteria or Acidobacteria, including class Acidobacteriia, Blastocatellia, Holophagae, Thermoanaerobaculia, or Vicinamibacteria, including order Acidobacteriales, Bryobacterales, Blastocatellales, Acanthopleuribacterales, Holophagales, Thermotomaculales, Thermoanaerobaculales, or Vicinamibacteraceae; phylum Actinobacteria, including class Acidimicrobiia, Actinobacteria, Actinomarinidae, Coriobacteriia, Nitriliruptoria, Rubrobacteria, or Thermoleophilia, including orders Acidimicrobiales, Acidothermales, Actinomycetales, Actinopolysporales, Bifidobacteriales, Nanopelagicales, Catenulisporales, Corunebacteriales, Cryptosporangiales, Frankiales, Geodermatophilales, Glycomycetales, Jiangellales, Micrococcales, Micromonosporales, Nakamurellales, Propionibacteriales, Pseudonocardiales, Sporichthyales, Streptomycetales, Streptosporangiales, Actinomarinales, Coriobacteriales, Eggerthellales, Egibacterales, Egicoccales, Euzebyales, Nitriliruptorales, Gaiellales, Rubrobacterales, Solirubrobacterales, or Thermoleophilales; phylum Aquificae, including class Aquificae, including order Aquificales or Desulfurobacteriales; phylum Armatimonadetes, including class Armatimonadia, including order Armatimonadales, Capsulimonadales, Chthonomonadetes, Chthonomonadales, Fimbriimonadia, or Fimbriimonadales; phylum Aureabacteria or Bacteroidetes, including class Armatimonadia, Bacteroidia, Chitinophagia, Cytophagia, Flavobacteria, Saprospiria or Sphingobacteriia, including order Bacteroidales, Marinilabiliales, Chitinophagales, Cytophagales, Flavobacteriales, Saprospirales, or Sphingopacteriales; phylum Balneolaeota, Caldiserica, Calditrichaeota, or Chlamydiae, including class Balneolia, Caldisericia, Calditrichae, or Chlamydia, including order Balneolales, Caldisericales, Calditrichales, Anoxychlamydiales, Chlamydiales, or Parachlamydiales; phylum Chlorobi or Chloroflexi, including class Chlorobia, Anaerolineae, Ardenticatenia, Caldilineae, Thermofonsia, Chloroflexia, Dehalococcoidia, Ktedonobacteria, Tepidiformia, Thermoflexia, Thermomicrobia, or Sphaerobacteridae, including order Chlorobiales, Anaerolineales, Ardenticatenales, Caldilineales, Chloroflexales, Herpetosiphonales, Kallotenuales, Dehalococcoidales, Dehalogenimonas, Ktedonobacterales, Thermogemmatisporales, Tepidiformales, Thermoflexales, Thermomicrobiales, or Sphaerobacterales; phylum Chrysiogenetes, Cloacimonetes, Coprothermobacterota, Cryosericota, or Cyanobacteria, including class Chrysiogenetes, Coprothermobacteria, Gloeobacteria, or Oscillatoriophycideae, including order Chrysiogenales, Coprothermobacterales, Chroococcidiopsidales, Gloeoemargaritales, Nostocales, Pleurocapsales, Spirulinales, Synechococcales, Gloeobacterales, Chroococcales, or Oscillatoriales; phyla: Eferribacteres, Deinococcus-thermus, Dictyoglomi, Dormibacteraeota, Elusimicrobia, Eremiobacteraeota, Fermentibacteria, or Fibrobacteres, including class Deferribacteres, Deinococci, Dictyoglomia, Elusimicrobia, Endomicrobia, Chitinispirillia, Chitinivibrionia, or Fibrobacteria, including order Deferribacterales, Deinococcales, Thermales, Dictyoglomales, Elusimicrobiales, Endomicrobiales, Chitinspirillales, Chitinvibrionales, Fibrobacterales, or Fibromonadales; phylum Firmicutes, Fusobacteria, Gemmatimonadetes, or Hydrogenedentes, including class Bacilli, Clostridia, Erysipelotrichia, Limnochordia, Negativicutes, Thermolithobacteria, Tissierellia, Fusobacteriia, Gemmatimonadetes, Longimicrobia, including order Bacillales, Lactobacillales, Borkfalkiales, Clostridiales, Halanaerobiales, Natranaerobiales, Thermoanaerobacterales, Erysipelotrichales, Limnochordales, Acidaminococcales, Selenomonadales, Veillonellales, Thermolithobacterales, Tissierellales, Fusobacteriales, Gemmatimonadales, or Longimicrobia; phylum Hydrogenedentes, Ignavibacteriae, Kapabacteria, Kiritimatiellaeota, Krumholzibacteriota, Kryptonia, Latescibacteria, LCP-89, Lentisphaerae, Margulisbacteria, Marinimicrobia, Melainabacteria, Nitrospinae, or Omnitrophica, including class Ignavibacteria, Kiritimatiellae, Krumholzibacteria, Lentisphaeria, Oligosphaeria, or Nitrospinae, including order Ignavibacteriales, Kiritimatiellales, Krumholzibacteriales, Lentisphaerales, Victivallales, Oligosphaerales, or Nitrospinia; phylum Omnitrophica or Planctomycetes, including class Brocadiae, Phycisphaerae, Planctomycetia, or Phycisphaerales, including order Sedimentisphaerales, Tepidisphaerales, Gemmatales, Isosphaerales, Pirellulales, or Planctomycetales; phylum Proteobacteria including class Acidithiobacillia, Alphaproteobacteria, Betaproteobacteria, Lambdaproteobacteria, Muproteobacteria, Deltaproteobacteria, Epsilonproteobacteria, Gammaproteobacteria, Hydrogenophilalia, Oligoflexia, or Zetaproteobacteria, including order Acidithiobacillales, Caulobacterales, Emcibacterales, Holosporales, Iodidimonadales, Kiloniellales, Kopriimonadales, Kordiimonadales, Magnetococcales, Micropepsales, Minwuiales, Parvularculales, Pelagibacterales, Rhizobiales, Rhodobacterales, Rhodospirillales, Rhodothalassiales, Rickettsiales, Sneathiellales, Sphingomonadales, Burkholderiales, Ferritrophicales, Ferrovales, Neisseriales, Nitrosomonadales, Procabacteriales, Rhodocyclales, Bradymonadales, Acidulodesulfobacterales, Desulfarculales, Desulfobacterales, Desulfovibrionales, Desulfurellales, Desulfuromonadales, Myxococcales, Syntrophobacterales, Campylobacterales, Nautiliales, Acidiferrobacterales, Aeromonadales, Alteromonadales, Arenicellales, Cardiobacteriales, Cellvibrionales, Chromatiales, Enterobacterales, Immundisolibacterales, Legionellales, Methylococcales, Nevskiales, Oceanospirillales, Orbales, Pasteurellales Pseudomonadales, Salinisphaerales, Thiotrichales, Vibrionales, Xanthomonadales, Hydrogenophilales, Bacteriovoracales, Bdellovibrionales, Oligoflexales, Silvanigrellales, or Mariprofundales; phylum Rhodothermaeota, Saganbacteria, Sericytochromatia, Spirochaetes, Synergistetes, Tectomicrobia, or Tenericutes, including class Rhodothermia, Spirochaetia, Synergistia, Izimaplasma, or Mollicutes, including order Rhodothermales, Brachyspirales, Brevinematales, Leptospirales, Spirochaetales, Synergistales, Acholeplasmatales, Anaeroplasmatales, Entomoplasmatales, or Mycoplasmatales; phylum Thermodesulfobacteria, Thermotogae, Verrucomicrobia, or Zixibacteria, including class Thermodesulfobacteria, Thermotogae, Methylacidiphilae, Opitutae, Spartobacteria, or Verrucomicrobiae, including order Thermodesulfobacteriales, Kosmotogales, Mesoaciditogales, Petrotogales, Thermotogales, Methylacidiphilales, Opitutales, Puniceicoccales, Xiphinematobacter, Chthoniobacterales, Terrimicrobium, or Verrucomicrobiales.

In other embodiments, the gene for the enzyme or regulatory protein is derived from an archaeon. It is envisioned that an enzyme or regulatory protein derived from any archaeon now known or later discovered can be utilized in the present invention. For example, the archaeon can be from phylum Euryarchaeota, including class Archaeoglobi, Hadesarchaea, Halobacteria, Methanobacteria, Methanococci, Methanofastidiosa, Methanomicrobia, Methanopyri, Nanohaloarchaea, Theionarchaea, Thermococci, or Thermoplasmata, including order Archaeoglobales, Hadesarchaeales, Halobacteriales, Methanobacteriales, Methanococcales, Methanocellales, Methanomicrobiales, Methanophagales, Methanosarcinales, Methanopyrales, Thermococcales, Methanomassiliicoccales, Thermoplasmatales, or Nanoarchaeales; DPANN superphylum, including subphyla Aenigmarcheota, Altiarchaeota, Diapherotrites, Micrarchaeota, Nanoarchaeota, Pacearchaeota, Parvarchaeota, or Woesearchaeota; TACK superphylum, including subphylum Korarchaeota, Crenarchaeota, Aigarchaeota, Geoarchaeota, Thaumarchaeota, or Bathyarchaeota; Asgard superphylum including subphylium Odinarchaeota, Thorarchaeota, Lokiarchaeota, Helarchaeota, or Heimdallarchaeota.

In additional embodiments, the gene for the enzyme or regulatory protein is derived from a fungus. It is envisioned that an enzyme or regulatory protein derived from any fungus now known or later discovered can be utilized in the present invention. This includes but is not limited to the phyla Chytridiomycota, Basidiomycota, Ascomycota, Blastocladiomycota, Ascomycota, Microsporidia, Basidiomycota, Glomeromycota, Symbiomycota, and Neocallimastigomycota. For example, the fungus can be from the phylum Ascomycota, including classes and orders Pezizomycotina, Arthoniomycetes, Coniocybomycetes, Dothideomycetes, Eurotiomycetes, Geoglossomycetes, Laboulbeniomycetes, Lecanoromycetes, Leotiomycetes, Lichinomycetes, Orbiliomycetes, Pezizomycetes, Sordariomycetes, Xylonomycetes, Lahmiales, Itchiclahmadion, Triblidiales, Saccharomycotina, Saccharomycetes, Taphrinomycotina, Archaeorhizomyces, Neolectomycetes, Pneumocystidomycetes, Schizosaccharomycetes, Taphrinomycetes; phylum Basidiomycota including subphyla or classes Pucciniomycotina, Ustilaginomycotina, Wallemiomycetes, and Entorrhizomycetes; subphylum Agaricomycotina including classes Tremellomycetes, Dacrymycetes, and Agaricomycetes; phylum Symbiomycota, including class Entorrhizomycota; subphylum Ustilaginomycotina including classes Ustilaginomycetes and Exobasidiomycetes; phylum Glomeromycota including classes Archaeosporomycetes, Glomeromycetes, and Paraglomeromycetes; subphylum Pucciniomycotina including orders and classes: Pucciniomycotina, Cystobasidiomycetes, Agaricostilbomycetes, Microbotryomycetes, Atractiellomycetes, Classiculomycetes, Mixiomycetes, and Cryptomycocolacomycetes; subphylum incertae sedis Mucoromyceta including orders Calcarisporiellomycota and Mucoromycota; phylum Mortierellomyceta including class Mortierellomycota; subphylum incertae sedis Entomophthoromycotina including order Entomophthorales; phylum Zoopagomyceta including classes Basidiobolomycota, Entomophthoromycota, Kickxellomycota, and Zoopagomycotina; subphylum incertae sedis Mucoromycotina including orders Mucorales, Endogonales, and Mortierellales; phylum Neocallimastigomycota including class Neocallimastigomycetes; phylum Blastocladiomycota including classes Physodermatomycetes and Blastocladiomycetes; phylum Rozellomyceta including classes Rozellomycota and Microsporidia; phylum Aphelidiomyceta including class Aphelidiomycota; Chytridiomyceta including classes Chytridiomycetes and Monoblepharidomycetes; and phylum Oomycota including classes or orders Leptomitales, Myzocytiopsidales, Olpidiopsidales, Peronosporales, Pythiales, Rhipidiales, Salilagenidiales, Saprolegniales, Sclerosporales, Anisolpidiales, Lagenismatales, Rozellopsidales, and Haptoglossales.

In additional embodiments, the gene for the enzyme or regulatory protein is derived from the organism below. This includes but is not limited to: Acanthurus tractus, Aplysina aerophoba, Bos Taurus, Bufo bufo, Bufotes viridis, Chrysochloris asiatica, Fukomys damarensis, Homo sapiens, Rattus norvegicus, Rhinella marina, Rhinella spinulosa, Schistosoma mansoni, Xenopus laevis, Xenopus tropicalis, Acacia koa, Arabidopsis thaliana, Brassica oleracea, Citrus sinensis, Hordeum vulgare, Juglans cinereal, Lophophora williamsii, Nymphaea colorata, Oryza sativa, Ricinus communis, Solanum lycopersicum, Sorghum bicolor, Theobroma cacao, and Triticum aestivum.

In some embodiments, the nucleic acids are codon optimized to improve expression, e.g., using techniques as disclosed in U.S. Pat. No. 10,435,727. More specifically, optimized nucleotide sequences are generated based on a number of considerations: (1) For each amino acid of the recombinant polypeptide to be expressed, a codon (triplet of nucleotide bases) is selected based on the frequency of each codon in the Saccharomyces cerevisiae genome; the codon can be chosen to be the most frequent codon or can be selected probabilistically based on the frequencies of all possible codons. (2) In order to prevent DNA cleavage due to a restriction enzyme, certain restriction sites are removed by changing codons that cover those sites. (3) To prevent lowcomplexity regions, long repeats (sequences of any single base longer than five bases) are modified. (2) and (3) are performed recursively to ensure that codon modification does not lead to additional undesirable sequences. (4) A ribosome binding site is added to the N-terminus. (5) A stop codon is added. (6) A localization signal is removed or replaced.

In some of the above embodiments, the nucleic acid provided herein comprises the sequence of any one of SEQ ID NOs:1-289.

In various embodiments, the nucleic acids further comprise additional nucleic acids encoding amino acids that are not part of the included enzymes or regulatory proteins herein. In some of these embodiments, the additional sequences encode additional amino acids present when the nucleic acid is translated, encoding, for example, a cofolding peptide, as previously discussed, or an additional protein domain, with or without a linker sequence, creating a fusion protein. Other examples are localization sequences, i.e., signals directing the localization of the folded protein to a specific subcellular compartment or membrane. Additional nonlimiting examples are an affibody tag, a localization scaffold, a vacuolar localization tag, a secretion signal, and a 6xhis tag.

In some embodiments, the nucleic acid comprises additional nucleotide sequences that are not translated. Nonlimiting examples include promoters, terminators, barcodes, Kozak sequences, targeting sequences, and enhancer elements. Particularly useful here are promoters that are functional in yeast.

Expression of a gene encoding an enzyme or regulatory protein is determined by the promoter controlling the gene. In order for a gene to be expressed, a promoter must be present within 1,000 nucleotides upstream of the gene. A gene is generally cloned under the control of a desired promoter. The promoter regulates the amount of enzyme expressed in the cell and also the timing of expression, or expression in response to external factors such as sugar source.

Any promoter now known or later discovered can be utilized to drive the expression of the enzymes and regulatory proteins described herein. See e.g. http://parts.igem.org/Yeast for a listing of various yeast promoters. Exemplary promoters listed in Table 3 below drive strong expression, constant gene expression, medium or weak gene expression, or inducible gene expression. Inducible or repressible gene expression is dependent on the presence or absence of a certain molecule. For example, the GAL1, GAL7, and GAL10 promoters are activated by the presence of the sugar galactose and repressed by the presence of the sugar glucose. The HO promoter is active and drives gene expression only in the presence of the alpha factor peptide. The HXT1 promoter is activated by the presence of glucose while the ADH2 promoter is repressed by the presence of glucose.

TABLE 3

Exemplary yeast promoters

| | | |
|---|---|---|
| Strong constitutive promoters | Medium and weak constitutive promoters | Inducible/repressible promoters |
| TEF1 | STE2 | GALI |
| PGK1 | TPI1 | GAL7 |
| PGI1 | PYK1 | GAL10 |
| TDH3 | | HO |
| | | HXT1 |
| | | ADH2 |

In various embodiments, the nucleic acid is in an expression cassette, e.g., a yeast expression cassette. Any yeast expression cassette capable of expressing the enzyme in a yeast cell can be utilized.

Additional regulatory elements can also be present in the expression cassette, including restriction enzyme cleavage sites, antibiotic resistance genes, integration sites, auxotrophic selection markers, origins of replication, and degrons.

The expression cassette can be present in a vector that, when transformed into a host cell, either integrates into chromosomal DNA or remains episomal in the host cell. Such vectors are well-known in the art. See e.g. http://parts.igem.org/Yeast for a listing of various yeast vectors.

A nonlimiting example of a yeast vector is a yeast episomal plasmid (YEp) that contains the pBluescript II SK(+) phagemid backbone, an auxotrophic selectable marker, yeast and bacterial origins of replication and multiple cloning sites enabling gene cloning under a suitable promoter (see Table 3). Other exemplary vectors include pRS series plasmids.

### Host Cells

The present invention is also directed to genetically engineered host cells that comprise the above-described nucleic acids. Such cells may be, e.g., any species of filamentous fungus, including but not limited to any species of Aspergillus, which have been genetically altered to produce precursor molecules, intermediate molecules, or cannabinoid molecules. Host cells may also be any species of bacteria, including but not limited to Escherichia, Corynebacterium, Caulobacter, Pseudomonas, Streptomyces, Bacillus, or Lactobacillus.

In some embodiments, the genetically engineered host cell is a yeast cell, which may comprise any of the above-described expression cassettes, and capable of expressing the recombinant enzyme encoded therein.

Any yeast cell capable of being genetically engineered can be utilized in these embodiments. Nonlimiting examples of such yeast cells include species of Saccharomyces, Candida, Pichia, Schizosaccharomyces, Scheffersomyces, Blakeslea, Rhodotorula, or Yarrowia.

These cells can achieve gene expression controlled by inducible promoter systems; natural or induced mutagenesis, recombination, and/or shuffling of genes, pathways, and whole cells performed sequentially or in cycles; overexpression and/or deletion of single or multiple genes and reducing or eliminating parasitic side pathways that reduce precursor concentration.

The host cells of the recombinant organism may also be engineered to produce any or all precursor molecules necessary for the biosynthesis of substituted indoles, tryptophans and tryptamines.

Construction of Saccharomyces cerevisiae strains expressing the enzymes and regulatory proteins provided herein is carried out via expression of a gene which encodes for the enzyme. The gene encoding the enzyme can be cloned into vectors with the proper regulatory elements for gene expression (e.g. promoter, terminator) and the derived plasmid can be confirmed by DNA sequencing. As an alternative to expression from an episomal plasmid, the gene encoding the enzyme may be inserted into the recombinant host genome. Integration may be achieved by a single or double cross-over insertion event of a plasmid, or by nuclease-based genome editing methods, as are known in the art e.g. CRISPR, TALEN and ZFR. Strains with the integrated gene can be screened by rescue of auxotrophy and genome sequencing. See, e.g., Green and Sambrook (2012).

FIGS. 9-15 provide nonlimiting examples of host cells utilizing the nucleic acids provided herein.

In some embodiments, the recombinant microorganism expresses TPH, TrpM, and AADC, where the recombinant microorganism produces at least one hydroxy substituted tryptamine compound, e.g., bufotenine, 5-OH-NMT, or 5-OH-TMT (FIG. 9, Panel A).

In other embodiments, the recombinant microorganism expresses TPH, TrpM, AADC, and IOMT, where the recombinant microorganism produces at least one methoxy substituted tryptamine compound, e.g., 5-MeO-NMT, 5-MeO-DMT, or 5-MeO-TMT (FIG. 9, Panel B).

In additional embodiments, the recombinant microorganism expresses AADC, T5H or T5H-CPR and INMT, where the recombinant microorganism produces at least one hydroxy substituted tryptamine compound, e.g., bufotenine, 5-OH-NMT, or 5-OH-TMT (FIG. 9, Panel C).

In further embodiments, the recombinant microorganism expresses AADC, T5H or T5H-CPR, INMT, and IOMT, where the recombinant microorganism produces at least one methoxy substituted tryptamine compound, e.g., 5-MeO-NMT, 5-MeO-DMT, or 5-MeO-TMT (FIG. 9, Panel D).

In other embodiments, the recombinant microorganism expresses TrpM and TPH, where the recombinant microorganism produces at least one hydroxy substituted tryptophan compound, e.g., 5-HTP, 5-OH-NMTP, 5-OH-DMTP or 5-OH-TMTP.

In additional embodiments, the recombinant microorganism expresses TrpM, TPH and IOMT, where the recombinant microorganism produces at least one methoxy substituted tryptophan compound, e.g., 5-MeO-NMTP, 5-MeO-DMTP or 5-MeO-TMTP.

In further embodiments, the recombinant microorganism expresses INMT and T5H, where the recombinant microorganism produces at least one hydroxy substituted tryptamine compound, e.g., bufotenine, 5-OH-NMT, or 5-OH-TMT.

In other embodiments, the recombinant microorganism expresses INMT, T5H and IOMT, where the recombinant microorganism produces at least one methoxy substituted tryptamine compound, e.g., 5-MeO-NMT, 5-MeO-DMT, or 5-MeO-TMT.

In additional embodiments, the recombinant microorganism expresses INMT, where the recombinant microorganism produces at least one hydroxy substituted tryptophan compound, e.g., 5-OH-NMTP, 5-OH-DMTP or 5-OH-TMTP.

In further embodiments, the recombinant microorganism expresses INMT and IOMT, where the recombinant microorganism produces at least one methoxy substituted tryptophan compound, e.g., 5-MeO-NMTP, 5-MeO-DMTP or 5-MeO-TMTP.

In other embodiments, the recombinant microorganism expresses INMT and AADC, where the recombinant microorganism produces at least one hydroxy substituted tryptamine compound, e.g., bufotenine, 5-OH-NMT, or 5-OH-TMT.

In additional embodiments, the recombinant microorganism expresses INMT, AADC and IOMT, where the recombinant microorganism produces at least one methoxy substituted tryptamine compound, e.g., 5-MeO-NMT, 5-MeO-DMT, or 5-MeO-TMT.

In further embodiments, the recombinant microorganism expresses INMT, where the recombinant microorganism produces at least one hydroxy substituted tryptamine compound, e.g., bufotenine, 5-OH-NMT, or 5-OH-TMT.

In other embodiments, the recombinant microorganism expresses INMT and IOMT, where the recombinant microorganism produces at least one methoxy substituted tryptamine compound, e.g., 5-MeO-NMT, 5-MeO-DMT, or 5-MeO-TMT.

In additional embodiments, the recombinant microorganism expresses INMT, where the recombinant microorganism produces at least one methoxy substituted tryptamine compound, e.g., 5-MeO-NMT, 5-MeO-DMT, or 5-MeO-TMT.

As depicted in FIG. 11, in some embodiments, the recombinant microorganism expresses AADC, IOMT, T5H or T5H-CPR, and NAT, where the recombinant microorganism produces a compound in the melatonin pathway, e.g., serotonin or melatonin. In some of these embodiments, the enzymes are on a scaffold to facilitate pathway throughput.

As depicted in FIG. 12, in some embodiments, the recombinant microorganism expresses AADC, T4H or T4H-CPR, PsiK and INMT (PsiM), where the recombinant microorganism produces a compound in the psilocybin pathway, e.g., baeocystin, psilocybin or aeruginascin. In some of these embodiments, the enzymes are on a scaffold to facilitate pathway throughput.

In accordance with the present invention, a recombinant host may also be modified to increase the accumulation of the methyl donor, SAMe, which is used by the recombinant TrpM and INMT enzymes to methylate indole and tryptamine molecules such as L-tryptophan and NMT. SAMe accumulation in the recombinant host cell may be increased by: (d) overexpressing enzymes to promote conversion of L-methionine to SAMe; (e) deleting off-pathway genes that encode for enzymes that deplete SAMe for unwanted side products; and (f) overexpressing a permease, which enables exogenous L-methionine to be fed to and transported into the cells.

The TrpM and INMT methyltransferase reactions consume one equivalent of adenosine triphosphate (ATP) and of SAMe. SAMe is a robust methyl donor synthesized from methionine and ATP via the L-methionine adenosyltransferase enzyme, Sam2. Sam2 may be overexpressed in a recombinant host to increase the conversion of L-methionine to SAMe. To support the increased pathway flux and generate more ATP, the adenylate kinase enzyme, Adk1, may also be overexpressed. To increase the uptake of exogenous L-methionine to feed into the SAMe pathway, recombinant Mup1, which is a methionine transporter, may be overexpressed.

SAMe is a precursor molecule for spermidine production and glycogen biosynthesis. To keep SAMe levels high in the pathways of the recombinant host and decrease off-pathway usage of SAMe, the SPE2 gene may be deleted in the recombinant host, thereby blocking the conversion of SAMe to spermidine. Glycogen biosynthesis consumes ATP, which is required for the conversion of L-methionine to SAMe. The gene encoding the enzyme Glc3 may be deleted in the recombinant host, thereby reducing production of glycogen, maintaining higher levels of ATP in the host cell, and increasing on-pathway flux of SAMe for methyltransferase activity. FIG. 10 depicts a recombinant host modified to express the enzymes enabling uptake and biosynthesis of indole and tryptamine precursors and the enzymes to create tryptamine, DMTP, DMT, and related substituted indole and tryptamine compounds.

### Recombinant Enzymes and Regulatory Proteins

The present invention is also directed to a non-naturally occurring enzyme or regulatory protein comprising an amino acid sequence encoded by any of the nucleic acids described above. In some embodiments, the amino acid sequence is 85%, 90%, 95%, 98%, or 100% identical to any one of SEQ ID NO:290-578. In these embodiments, the enzyme or regulatory protein can be isolated in vitro and used in vitro to provide enzyme activity. Alternatively, as discussed above, the enzyme can be expressed in a recombinant organism, e.g., a microorganism or a plant. In some of these embodiments, the recombinant microorganism is a bacterium, for example an E. coli. In other embodiments, the recombinant microorganism is a yeast cell, e.g., a species of Saccharomyces (for example S. cerevisiae), Candida, Pichia, Schizosaccharomyces, Scheffersomyces, Blakeslea, Rhodotorula, Aspergillus or Yarrowia.

### Methods

The systems and methods herein include: (i) growing modified recombinant host cells and thereby yielding a recombinant host organism; (ii) expressing engineered indole and tryptamine biosynthesis genes and enzymes in the recombinant host organism; (iii) producing or synthesizing substituted indoles and tryptamines in the recombinant host organism; (iv) fermenting the recombinant host organism; and (v) isolating the substituted indoles and tryptamines from the recombinant host organism. Endogenous pathways of the recombinant host can be modified by the systems and methods herein to produce high purity substituted indoles and tryptamines.

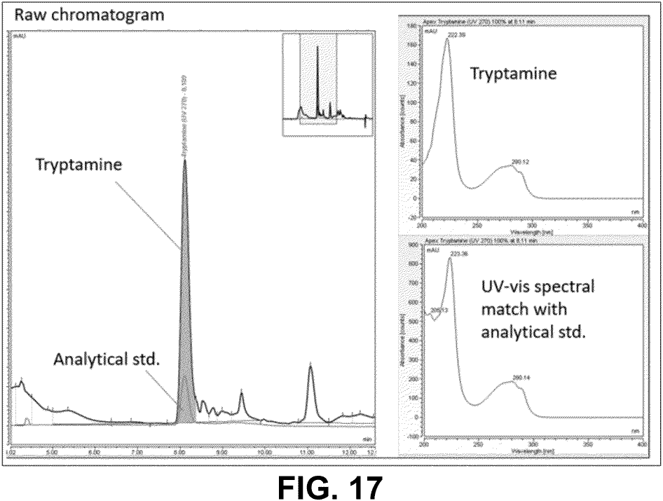

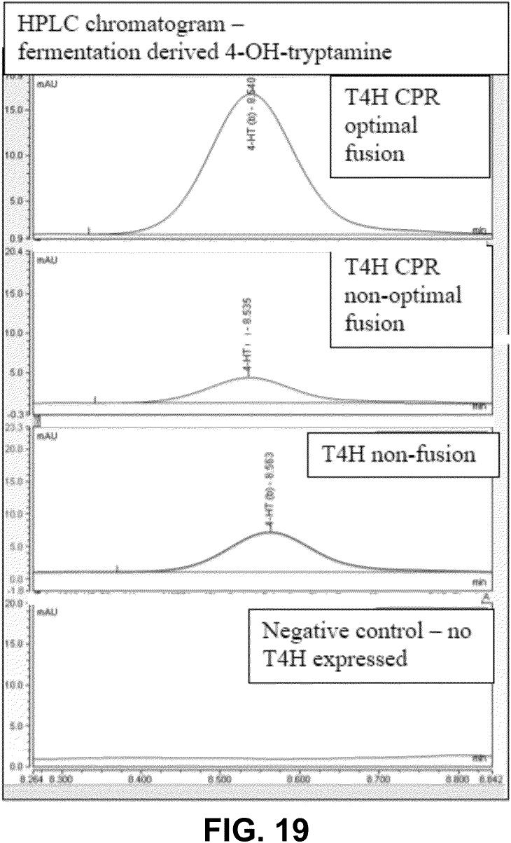

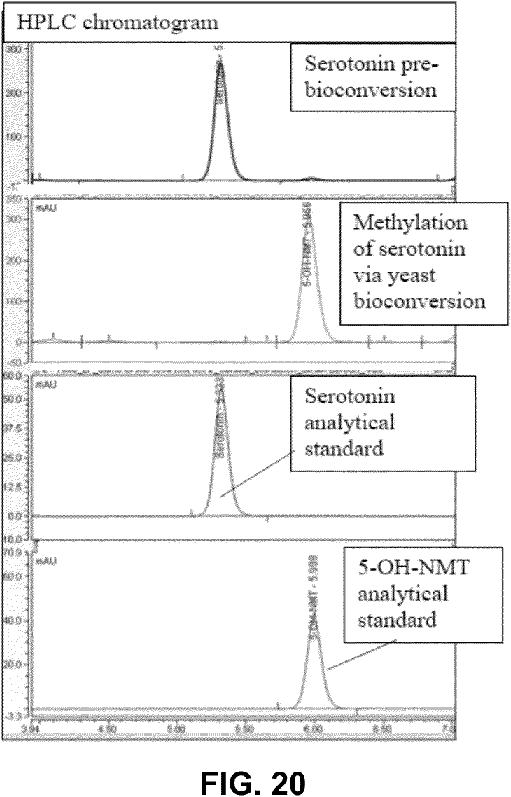

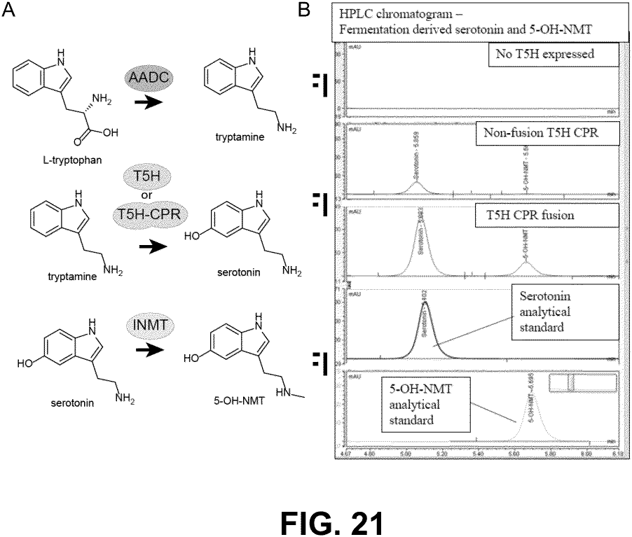

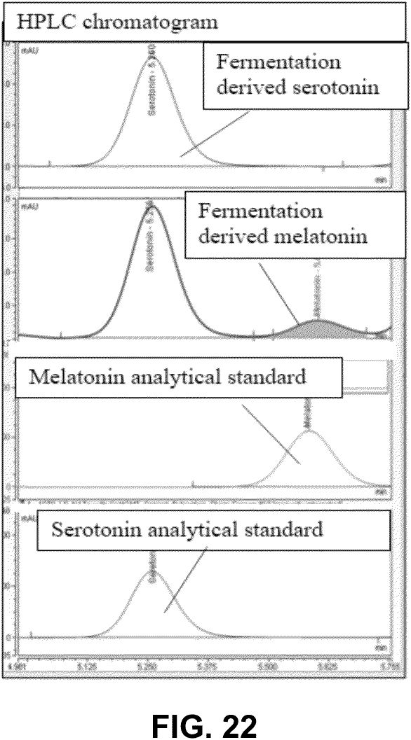

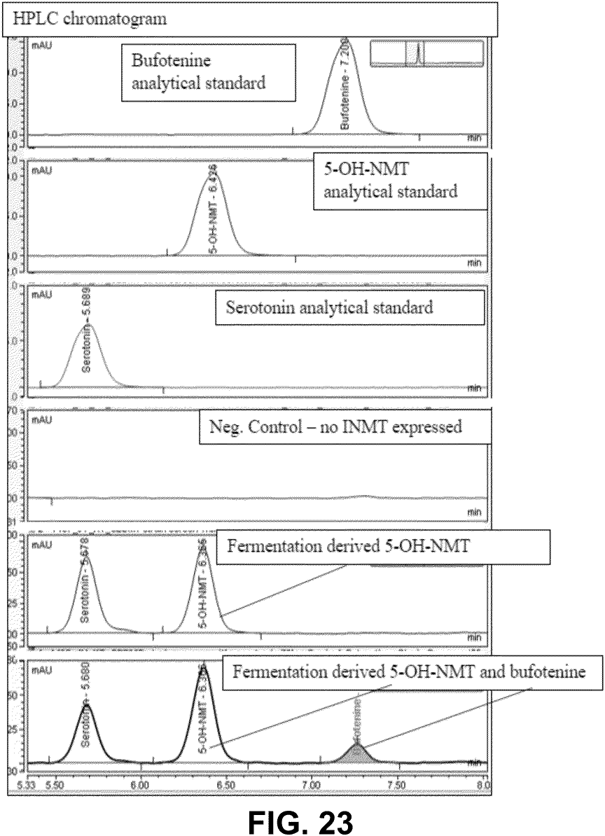

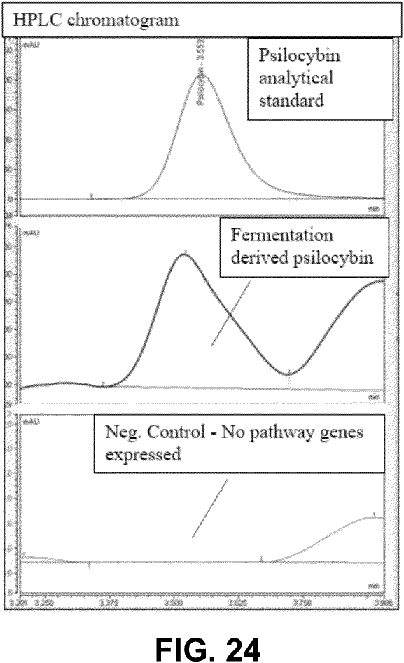

To produce the desired substituted indole, the nucleic acid encoding the enzymes and/or regulatory proteins are introduced into a host cell using standard cell (e.g., yeast) transformation techniques (Green and Sambrook, 2012). Cells are subjected to fermentation under conditions that activate the promoter controlling the synthesis of the enzyme and/or regulatory protein. The broth may be subsequently subjected to HPLC analysis to determine the presence or yield of the desired substituted indole, as in FIGS. 17-24 and 26.

In various embodiments, the host cells are provided with various feedstocks to drive production of the desired substituted indole, e.g., glucose, fructose, sucrose, ethanol, fatty acids, glycerol, molasses, corn steep liquor, dairy, fish waste, etc. for example as discussed in U.S. Pat. Application 17/078636.

In some embodiments, for recombinant enzyme purification, the gene encoding the enzyme and/or regulatory protein is cloned into an expression vector such as the pET expression vectors from Novagen, transformed into a protease deficient strain of E. coli such as BL21 and expressed by induction with IPTG. The protein of interest may be tagged with an affinity tag to facilitate purification, e.g. hexahistidine, GST, calmodulin, TAP, AP, CAT, HA, FLAG, MBP etc. Coexpression of a bacterial chaperone such as dnaK, GroES/GroEL or SecY may help facilitate protein folding. See Green and Sambrook (2012).

Any of the enzymes and/or regulatory proteins described above can also be produced in transgenic plants, using techniques known in the art (see, e.g., Keshavareddy et al., 2018). In these embodiments, the above-described nucleic acid encoding the enzyme and/or regulatory protein further comprises a promoter functional in a plant. In various embodiments, the nucleic acid is in a plant expression cassette. Any plant capable of being transformed with the nucleic acid can be utilized here. In some embodiments, the plant is a tobacco or cannabis.

Preferred embodiments are described in the following examples. Other embodiments within the scope of the claims herein will be apparent to one skilled in the art from consideration of the specification or practice of the invention as disclosed herein. It is intended that the specification, together with the examples, be considered exemplary only, with the scope and spirit of the invention being indicated by the claims, which follow the examples.

In the examples below, genetically engineered host cells may be any species of yeast herein, including but not limited to any species of Saccharomyces, Candida, Schizosaccharomyces, Yarrowia, etc., which have been genetically altered to produce precursor molecules, intermediate molecules, and psilocybin molecules. Additionally, genetically engineered host cells may be any species of filamentous fungus, including but not limited to any species of Aspergillus, which have been genetically altered to produce precursor molecules such as L-tryptophan and substituted indole and tryptamine molecules. Some of the species of yeast herein for the recombinant host organism include but are not limited to: Schizosaccharomyces cerevisiae, Schizosaccharomyces japonicus, Schizosaccharomyces pombe, Schizosaccharomyces cryophilus, Saccharomyces cerevisiae, Kluyveromyces lactis, Kluyveromyces dobzhanskii, and Yarrowia lipolytica.

In these examples, the gene sequences from gene source organisms are codon optimized to improve expression using techniques disclosed in U.S. Pat.10,435,727.

DNA sequences are synthesized and cloned using techniques known in the art. Gene expression can be controlled by inducible or constitutive promoter systems using the appropriate expression vectors. Genes are transformed into an organism using standard yeast or fungus transformation methods to generate modified host strains (i.e., the recombinant host organism). The modified strains express genes for: (i) producing L-tryptophan, SAMe and precursor molecules to L-tryptophan and SAMe; (ii) increasing an output of L-tryptophan molecules and precursor molecules to L-tryptophan and SAMe molecules; (iii) increasing the import of exogenous L-tryptophan, L-methionine, SAMe and TMG into the host strain; and (iv) the genes for biosynthetic pathways that generate DMT, DMTP, bufotenine, 5-MeO-DMT and all intermediate indole and tryptamine compounds synthesized and described herein. In the presence or absence of exogenous L-tryptophan, L-methionine, SAMe, TMG, 5-HTP, melatonin, and serotonin, fermentations are run to determine if the cell will convert the fed precursors into tryptamine, serotonin, methylated versions of serotonin, melatonin, or methylated versions of melatonin. The L-tryptophan, SAMe, hydroxylation, decarboxylation, and methylation pathway genes herein can be integrated into the genome of the cell or maintained as an episomal plasmid. Samples are: (i) prepared and extracted using a combination of fermentation, dissolution, and purification steps; and (ii) analyzed by HPLC for the presence of directing molecules (e.g., SAMe and L-tryptophan), precursor molecules, intermediate molecules, and target molecules such as bufotenine and 5-MeO-DMT.

Using the systems and methods herein, the genes which can be expressed to encode for a corresponding enzyme or other type of proteins include but are not limited to: ENO2, TAL1, ARO1, ADK1, MUP1, SAM2, MHT1, SAM4, SAM3, TAT2, AADC, TRPM, INMT, TPH, genes encoding enzymes for the BH4 biosynthesis pathway, genes encoding enzymes for the BH4 regeneration pathway, T5H, IOMT, caffOMT, NAT, DAC, T4H, PsiK, oxidase, phosphatase, TrpHalo, DMAT, T4H-CPR, T5H-CPR, TrpS, and ATMT. For example, the AADC gene is expressed, or overexpressed, to encode for the aromatic amino decarboxylase enzyme; the TRPM gene is expressed to encode for the TrpM enzyme; and so forth. Gene sequences can be determined using standard techniques known in the art, e.g., the techniques disclosed in U.S. Pat. 10,671,632.

### EXAMPLES

### Example 1 - Construction of Saccharomyces Cerevisiae Platform Strains With Elevated Indole and Tryptamine Precursors

The construction of Saccharomyces cerevisiae platform strains with elevated metabolic flux towards L-tryptophan is carried out by overexpressing five optimized enzymes in or upstream of the shikimate pathway to make the aromatic compound intermediate, chorismate, and one optimized enzyme in the tryptophan pathway to make L-tryptophan. Further, tryptophan levels in the cell are enhanced with the expression of TAT2, a tryptophan importer, and L-tryptophan supplementation in the media up to 1% mass to volume. Finally, five enzymes are deleted in the cell to decrease off-pathway consumption of the L-tryptophan. The genetically modified host described herein can be the same host used for production of psilocybin and DMT as both production pathways use the precursor, L-tryptophan. A specific description of the strain with elevated L-tryptophan is disclosed in U.S. Pat. Publication 2021/0147888.

### Example 2 - Construction of Saccharomyces Cerevisiae Platform Strains With Synthesis of Methyl Donor

Construction of Saccharomyces cerevisiae platform strains with elevated SAMe production is carried out via expression of SAM2, a SAMe synthetase gene. The SAM2 gene is cloned from Saccharomyces cerevisiae using techniques known in the art. The gene can be cloned into vectors with the proper regulatory elements for gene expression (e.g. promoter, terminator) and the derived plasmid can be confirmed by DNA sequencing. As an alternative to expression from an episomal plasmid, the SAM2 gene is inserted into the recombinant host genome. Integration is achieved by a single cross-over insertion event of the plasmid. Strains with the integrated gene can be screened by rescue of auxotrophy and genome sequencing.

### Example 3 - Construction of Saccharomyces Cerevisiae Platform Strains With Elevated Methyl Donor Production

Construction of Saccharomyces cerevisiae platform strains with elevated SAMe production via expression of the ADK1, adenylate kinase gene. The ADK1 gene is cloned from Saccharomyces cerevisiae using techniques known in the art. The gene can be cloned into vectors with the proper regulatory elements for gene expression (e.g. promoter, terminator) and the derived plasmid can be confirmed by DNA sequencing. As an alternative to expression from an episomal plasmid, the ADK1 gene is inserted into the recombinant host genome. Integration is achieved by a single cross-over insertion event of the plasmid. Strains with the integrated gene can be screened by rescue of auxotrophy and genome sequencing.

Further SAM accumulation for methyl donor availability is achieved herein by engineering the homocysteine to methionine side of the methylation pathway. SAH is generated after methylation of serotonin and other intermediates to produce bufotenine and other compounds described herein. SAH is recycled back to methionine after methyl donation by TMG (trimethylglycine) or betaine. TMG is fed to the cells up to 1% (v/v) in the growth media. Two Saccharomyces cerevisiae genes, MHT1 and SAM4 encode the enzymes, Mht1 and Sam4, that are responsible for homocysteine re-methylation using TMG as a methyl donor. MHT1 and SAM4 are overexpressed from a high copy vector with a strong promoter.

### Example 4 - Construction of Saccharomyces Cerevisiae Platform Strains With Enhanced Uptake of Methyl Donor Precursors

Construction of Saccharomyces cerevisiae platform strains with elevated SAMe production is carried out via expression of MUP1, the methionine permease gene. The MUP1 gene is cloned from Saccharomyces cerevisiae using techniques known in the art. The gene can be cloned into vectors with the proper regulatory elements for gene expression (e.g. promoter, terminator) and the derived plasmid can be confirmed by DNA sequencing. As an alternative to expression from an episomal plasmid, the MUP1 gene is inserted into the recombinant host genome. Integration is achieved by a single cross-over insertion event of the plasmid. Strains with the integrated gene can be screened by rescue of auxotrophy and genome sequencing.

### Example 5 - Construction of Saccharomyces Cerevisiae Platform Strains With Enhanced Uptake of Methyl Donors

Herein we describe a strategy to increase the SAM accumulation by increasing transport of exogenous SAM into the cell. SAM levels are increased by overexpressing the gene, SAM3. SAM3 encodes for the Sam3 protein, the predominant Saccharomyces cerevisiae transporter that is responsible for SAM import. SAM3 is expressed from a high-copy vector with a strong promoter and media is supplemented with 0.5 - 1.0 mM SAMe.

### Example 6 - Construction of Saccharomyces Cerevisiae Platform Strains With Decreased Off-Pathway Flux of Methyl Donors

Construction of Saccharomyces cerevisiae platform strains with elevated metabolic flux towards SAMe is carried out via deletion of SPE2 to reduce SAMe decarboxylation. Deletion of SPE2 is performed by replacement of the SPE2 gene with the URA3 cassette in the recombinant host. The SPE2 URA3 knockout fragment, carrying the marker cassette, URA3, and homologous sequence to the targeted gene, SPE2, can be generated by bipartite PCR amplification. The PCR product is transformed into a recombinant host and transformants can be selected on synthetic URA drop-out media. Further verification of the modification in said strain can be carried out by genome sequencing, then analyzed by the techniques disclosed in U.S. Pat. 10,671,632.

### Example 7 - Construction of Saccharomyces Cerevisiae Platform Strains With Decreased Off-Pathway Flux of Methyl Donor Precursors

Saccharomyces cerevisiae platform strains are constructed with elevated metabolic flux towards SAMe via deletion of GLC3 to reduce ATP consumption. Deletion of GLC3 is performed by replacement of the GLC3 gene with the URA3 cassette in the recombinant host. The GLC3 URA3 knockout fragment, carrying the marker cassette, URA3, and homologous sequence to the targeted gene, GLC3, can be generated by bipartite PCR amplification. The PCR product is transformed into a recombinant host and transformants can be selected on synthetic URA drop-out media. Further verification of the modification in said strain can be carried out by genome sequencing and analyzed by the techniques disclosed in U.S. Pat. 10,671,632.

### Example 8 - Construction of Saccharomyces Cerevisiae Platform Strains With Increased Tryptophan Accumulation

Saccharomyces cerevisiae platform strains with accumulation of tryptophan are generated by deletion of BNA2. Bna2 is an enzyme necessary for de novo NAD+ production from tryptophan. Deletion of BNA2 is performed by replacement of the BNA2 gene with the URA3 cassette in the recombinant host. The BNA2 URA3 knockout fragment, carrying the marker cassette, URA3, and homologous sequence to the targeted gene, BNA2, can be generated by bipartite PCR amplification. The PCR product is transformed into a recombinant host and transformants can be selected on synthetic URA drop-out media. Further verification of the modification in said strain can be carried out by genome sequencing and analyzed by the techniques disclosed in U.S. Pat. 10,671,632.

### Example 9 - Expression of Recombinant L-Tryptophan Methyltransferases in a Modified Host Organism

Construction of Saccharomyces cerevisiae NMTP, DMTP, and TMTP production strains is carried out via expression of the TrpM methyltransferase gene. The optimized TrpM gene is synthesized using DNA synthesis techniques known in the art. The optimized gene can be cloned into vectors with the proper regulatory elements for gene expression (e.g. promoter, terminator) and the derived plasmid can be confirmed by DNA sequencing. As an alternative to expression from an episomal plasmid, the optimized TrpM gene is inserted into the recombinant host genome. Integration is achieved by a single cross-over insertion event of the plasmid. Strains with the integrated gene can be screened by rescue of auxotrophy and genome sequencing.

### Example 10 - Expression of Recombinant Aromatic Amino Acid Decarboxylases in a Modified Host Organism

Construction of Saccharomyces cerevisiae tryptamine production strains is carried out via expression of the AADC gene which encodes the enzyme that converts L-tryptophan to tryptamine. AACD also encodes the enzyme that converts 5HTP to serotonin. This specific conversion may be carried out by the same enzyme encoded by the AADC gene that converts L-tryptophan to tryptamine. It also may be carried out by the gene product of a novel AADC described herein. The optimized AADC gene is synthesized using DNA synthesis techniques known in the art. The optimized gene can be cloned into vectors with the proper regulatory elements for gene expression (e.g. promoter, terminator) and the derived plasmid can be confirmed by DNA sequencing. As an alternative to expression from an episomal plasmid, the optimized AADC gene is inserted into the recombinant host genome. Integration is achieved by a single cross-over insertion event of the plasmid. Strains with the integrated gene can be screened by rescue of auxotrophy and genome sequencing.

### Example 11 - Expression of Recombinant L-Tryptophan Hydroxylases in a Modified Host Organism

Construction of the Saccharomyces cerevisiae 5-HTP production strains is carried out via expression of the gene that encodes tryptophan hydroxylase. 5-HTP is a precursor compound for production of serotonin and variants described herein. Tryptophan hydroxylase activity is dependent on the availability of the BH4 cofactor. The optimized TPH, BH4 biosynthesis and BH4 regeneration genes are synthesized using DNA synthesis techniques known in the art. The optimized genes can be cloned into vectors with the proper regulatory elements for gene expression (e.g. promoter, terminator) and the derived plasmid can be confirmed by DNA sequencing. As an alternative to expression from an episomal plasmid, the optimized TPH, BH4 biosynthesis and BH4 regeneration genes are inserted into the recombinant host genome. Integration is achieved by a single cross-over insertion event of the plasmid. Strains with the integrated gene can be screened by rescue of auxotrophy and genome sequencing.

### Example 12 - Expression of Recombinant Tryptamine 5-Hydroxylases in a Modified Host Organism

Construction of the Saccharomyces cerevisiae serotonin production strains is carried out via expression of the gene that encodes tryptamine 5-hydroxylase (T5H). 5-HT or serotonin is a precursor compound for production of bufotenine and variants described herein. T5H activity is dependent on the availability of the intermediate indole compound, tryptamine, production of which is disclosed in U.S. Pat. Publication 2021/0147888 and further described herein.

T5H, as a cytochrome p450-containing monooxygenase, is also dependent on the cytochrome p450 reductase enzyme (CPR) for full activity. The CPR facilitates electron transfer from the NAD(P)H. The optimized T5H and CPR genes are synthesized using DNA synthesis techniques known in the art. The optimized genes can be cloned into vectors with the proper regulatory elements for gene expression (e.g. promoter, terminator) and the derived plasmid can be confirmed by DNA sequencing. As an alternative to expression from an episomal plasmid, the optimized T5H and CPR genes are inserted into the recombinant host genome. Integration is achieved by a single cross-over insertion event of the plasmid. Strains with the integrated gene can be screened by rescue of auxotrophy and genome sequencing.

### Example 13 - Expression of Recombinant Indolethylamine-N-Methyltransferase (INMT) in a Modified Host Organism

Construction of Saccharomyces cerevisiae DMT production strains is carried out via expression of the INMT gene which encodes the enzyme that methylates tryptamine to DMT. INMT also encodes the enzyme that converts serotonin to bufotenine. Finally, INMT encodes the enzyme that converts 5-MeO-tryptamine to 5-Meo-DMT. These unique conversions may be carried out by the same enzyme encoded by the INMT gene that converts tryptamine to DMT. It also may be carried out by the gene product of a novel INMT described herein. The optimized INMT gene is synthesized using DNA synthesis techniques known in the art. The optimized gene can be cloned into vectors with the proper regulatory elements for gene expression (e.g. promoter, terminator) and the derived plasmid can be confirmed by DNA sequencing. As an alternative to expression from an episomal plasmid, the optimized INMT gene is inserted into the recombinant host genome. Integration is achieved by a single cross-over insertion event of the plasmid. Strains with the integrated gene can be screened by rescue of auxotrophy and genome sequencing.

### Example 14 - Expression of 5-Hydroxyindole-O-Methyltransferase (IOMT) or Caffeic Acid-O-Methyltransferase (CaffOMT) in a Modified Host Organism

Construction of Saccharomyces cerevisiae 5-MeO-DMT production strains is carried out via expression of the IOMT gene which encodes the enzyme that methylates the 5-OH in bufotenine, an intermediate derived from the INMT conversion of serotonin, described herein. The IOMT gene also encodes for the enzyme that converts serotonin to 5-MeO-tryptamine in the first intermediate to make melatonin. The IOMT enzyme also methylates the 5-OH of N-acetyl-serotonin to generate melatonin as an intermediate to make 5-MeO-tryptamine and further, 5-MeO-DMT. Alternatively, the enzyme that converts serotonin to 5-MeO-tryptamine can be carried out with a CaffOMT enzyme, an enzyme shared with the phenylpropanoid biosynthesis pathway. This same CaffOMT enzyme can also methylate N-acetyl-serotonin to generate melatonin. The optimized IOMT or CaffOMT gene is synthesized using DNA synthesis techniques known in the art. The optimized gene can be cloned into vectors with the proper regulatory elements for gene expression (e.g. promoter, terminator) and the derived plasmid can be confirmed by DNA sequencing. As an alternative to expression from an episomal plasmid, the optimized IOMT or CaffOMT gene is inserted into the recombinant host genome. Integration is achieved by a single cross-over insertion event of the plasmid. Strains with the integrated gene can be screened by rescue of auxotrophy and genome sequencing.

### Example 15 - Expression of Recombinant N-acetyl Transferase (NAT) and Melatonin Deacetylase (NAD) in a Modified Host Organism

Construction of Saccharomyces cerevisiae 5-MeO-DMT production strains can alternatively be carried out via expression of the two more enzymes, NAT and NAD. NAT acetylation of serotonin produces the intermediate N-acetyl-serotonin or NAS. NAS is converted to melatonin with the IOMT (or CaffOMT) enzyme described herein. DAC deacetylases melatonin to 5-MeO-tryptamine which is converted to 5-MeO-DMT via the INMT enzyme described herein. The optimized NAT and DAC genes are synthesized using DNA synthesis techniques known in the art. The optimized gene can be cloned into vectors with the proper regulatory elements for gene expression (e.g. promoter, terminator) and the derived plasmid can be confirmed by DNA sequencing. As an alternative to expression from an episomal plasmid, the optimized NAT and DAC genes are inserted into the recombinant host genome. Integration is achieved by a single cross-over insertion event of the plasmid. Strains with the integrated gene(s) can be screened by rescue of auxotrophy and genome sequencing.

### Example 16 - Construction of Saccharomyces Cerevisiae Platform Strains With Accumulation of Serotonin

Serotonin is the precursor molecule for both bufotenine and 5-MeO-DMT. Construction of a Saccharomyces cerevisiae serotonin strain is carried out by expression of AADC and TPH or AADC and T5H genes described herein for the enzymatic conversion of L-tryptophan to serotonin. Exogenous serotonin is also fed to the strains to increase precursor levels at concentrations of 0.5 mM to 2 mM. Exogenous 5-HTP with expression of the AADC gene is fed to the cells as a mechanism to increase the serotonin precursor.

In order to accumulate serotonin in the cell and prevent off pathway conversion of serotonin to unwanted products, the endogenous Saccharomyces cerevisiae gene, PAA1 (YDR071C) is deleted. PAA1 is a polyamine acetyltransferase that would acetylate serotonin and use up valuable acetyl-CoA.

### Example 17 - Method of Growth

Modified host cells that yield substituted indoles and tryptamine compounds, such as a bufotenine-producing strain herein, express engineered bufotenine biosynthesis genes and enzymes. More specifically, the bufotenine-producing strain herein is grown in a minimal, complete culture media containing yeast nitrogen base, amino acids, vitamins, ammonium sulfate, and a carbon source of glucose and galactose. The recombinant host cells are grown in 24-well plates or shake flasks in a volume range of 2 mL to 100 mL of media starting from an inoculation density of OD₆₀₀ₙₘ=1. Exogenous serotonin, melatonin, tryptamine, 5HTP, SAMe and TMG can be added to media to supplement the precursor pool for final compound production or support methyl donor accumulation.

### Example 18 - Conversion of Melatonin to 5-Methoxy-Tryptamine Using a Bio-Based Enzyme Factory

Herein we describe a strategy for 5-methoxy-tryptamine (5-MT) production by recombinant expression and secretion of the melatonin deacetylase, DAC in BL21(DE3)pLysS E. coli. The DAC enzyme is cloned into a high-copy vector with key features that allow 1) tight induction by the lactose analog, β-D-thiogalactoside (IPTG) 2) an N-terminal secretory signal peptide [MKKTAIAIAVALAGFATVAQA (SEQ ID NO:286,575)] and 3) C-terminal fusion to a HIS tag for purification. E. coli cells harboring the NAD-expression vector are grown in M9 minimal media with 1% glucose for 18h at 37° C. and shaking at 300 rpm. Concentrated cell culture is diluted to an OD₆₀₀ =1 in fresh M9 minimal media with 1% glucose and 0.2 mM IPTG. After a 3 h induction at 18° C. and 300 rpm shaking, melatonin is added to the media at a final concentration of 1-2 mM. Cells are grown at room temperature for 48 h, shaking at 300 rpm. Media is collected at 24 h and 48 h and analyzed by HPLC as described herein.

Alternatively, we describe a strategy for production of the compound, 5-methoxy-tryptamine (5-MT) by recombinant expression and secretion of the melatonin deacetylase, DAC in Saccharomyces cerevisiae. The DAC enzyme is cloned into a high-copy vector with key features that allow 1) tight induction by the sugar, galactose 2) an N-terminal alpha factor secretion leader sequence, [MEGVSLEKREAEA (SEQ ID NO:574] and 3) c-terminal fusion to a HIS tag for purification. Saccharomyces cerevisiae cells harboring the DAC-expression vector are grown in CM minimal media with 2% glucose for 18 h at 30° C. and shaking at 300 rpm. Concentrated cell culture is diluted to an OD₆₀₀ =1 in fresh CM minimal media with 2% galactose. After 24 h of induction at 30° C. and 300 rpm shaking, melatonin is added to the media at a final concentration of 1-2 mM. Cells are grown at 30° C. and 300 rpm shaking for 48 h. Media is collected at 24 h and 48 h and analyzed by HPLC as described herein.

Alternatively, we describe a strategy for production of the compound, 5-methoxy-tryptamine (5-MT) by recombinant expression and secretion of the melatonin deacetylase (DAC) in Komagataella phaffii (Pichia pastoris). The DAC enzyme is cloned into a high-copy vector with key features that allow 1) induction by methanol with the AOX1 promoter and 2) a secretion signal consisting of the α-factor pro region. K. phaffii cells harboring the DAC enzyme are inoculated into 5 mL of YPD in a 15-mL culture tube. After a day of incubation at 30° C. with shaking at 220 rpm, an aliquot of the culture is diluted to an OD₆₀₀=0.2 in 5 mL of BMG (buffered minimal glycerol media) in a 15-mL culture tube. This tube is incubated under the same conditions as before. The following day, the culture is centrifuged at 3000 rpm (2000×g) for 5 min and resuspended in 25 mL BMM (buffered minimal methanol media) to attain an OD₆₀₀ = 1.0. 25 mL of this culture is placed in a 250-mL baffled flask, and during this induction phase, the cells are incubated at 25° C. with shaking at 150 rpm to reduce loss of methanol. After 1 day of induction, an additional dose of 125 µL methanol is added (yielding a final concentration of 0.5%), melatonin is added to the media at a final concentration of 1-2 mM, and the incubation is continued for another day. After 48 h of induction, media is collected at at 24 h and 48 h and analyzed by HPLC as described herein.

### Example 19 - Conversion of Melatonin to 5-Methoxy-NMT, 5-Methoxy-DMT, and 5-Methoxy-TMT Using a Bio-Based Enzyme Factory

Herein we describe a strategy for 5-MeO-NMT, 5-MeO-DMT, and 5-MeO-TMT production by recombinant expression and secretion of the indolethylamine-N-methyltransferase (INMT) in in BL21(DE3)pLysS E. coli. The INMT enzyme is cloned into a high-copy vector with key features that allow 1) tight induction by the lactose analog, β-D-thiogalactoside (IPTG) 2) an N-terminal secretory signal peptide [MKKTAIAIAVALAGFATVAQA (SEQ ID NO:574)] and 3) C-terminal fusion to a HIS tag for purification. E. coli cells harboring the INMT-expression vector are grown in M9 minimal media with 1% glucose for 18 h at 37° C. and shaking at 300 rpm. Concentrated cell culture is diluted to an OD₆₀₀ =1 in fresh M9 minimal media with 1% glucose and 0.2 mM IPTG. After a 3 h induction at 18° C. and 300 rpm shaking, melatonin is added to the media at a final concentration of 1-2 mM and SAMe is added to the media at a final concentration of 1-2 mM. Cells are grown at room temperature for 48 h, shaking at 300 rpm. Media is collected at 24h and 48h and analyzed by HPLC as described herein.

Alternatively, we describe a strategy for production of the compounds, 5-MeO-NMT, 5-MeO-DMT, and 5-MeO-TMT by recombinant expression and secretion of the indolethylamine-N-methyltransferase (INMT) in Saccharomyces cerevisiae. The INMT enzyme is cloned into a high-copy vector with key features that allow 1) tight induction by the sugar, galactose 2) an N-terminal alpha factor secretion leader sequence, [MEGVSLEKREAEA (SEQ ID NO:574)] and 3) c-terminal fusion to a HIS tag for purification. Saccharomyces cerevisiae cells harboring the INMT-expression vector are grown in CM minimal media with 2% glucose for 18 h at 30° C. and shaking at 300 rpm. Concentrated cell culture is diluted to an OD₆₀₀ =1 in fresh CM minimal media with 2% galactose. After 24 h of induction at 30° C. and 300 rpm shaking, melatonin is added to the media at a final concentration of 1-2 mM and SAMe is added to the media at a final concentration of 1-2 mM. Cells are grown at 30° C. and 300 rpm shaking for 48h. Media is collected at 24 h and 48 h and analyzed by HPLC as described herein.

Alternatively, we describe a strategy for production of the compounds, 5-MeO-NMT, 5-MeO-DMT, and 5-MeO-TMT by recombinant expression and secretion of the indolethylamine-N-methyltransferase (INMT) in Komagataella phaffii. The INMT enzyme is cloned into a high-copy vector with key features that allow 1) induction by methanol with the AOX1 promoter and 2) a secretion signal consisting of the α-factor pro region. K. phaffii cells harboring the DAC enzyme are inoculated into 5 mL of YPD in a 15-mL culture tube. After a day of incubation at 30° C. with shaking at 220 rpm, an aliquot of the culture is diluted to an OD₆₀₀=0.2 in 5 mL of BMG (buffered minimal glycerol media) in a 15-mL culture tube. This tube is incubated under the same conditions as before. The following day, the culture is centrifuged at 3000 rpm (2000×g) for 5 min and resuspended in 25 mL BMM (buffered minimal methanol media) to attain an OD₆₀₀ = 1.0. 25 mL of this culture is placed in a 250-mL baffled flask, and during this induction phase, the cells are incubated at 25° C. with shaking at 150 rpm to reduce loss of methanol. After 1 day of induction, an additional dose of 125 µL methanol is added (yielding a final concentration of 0.5%), melatonin is added to the media at a final concentration of 1-2 mM, SAMe is added to the media at a final concentration of 1-2 mM, and the incubation is continued for another day. After 48 h of induction, media is collected at 24 h and 48 h and analyzed by HPLC as described herein.

### Example 20 - Conversion of Tryptamine to NMT, DMT, and TMT Using a Bio-Based Enzyme Factory

Herein we describe a strategy for NMT, DMT, and TMT production by recombinant expression and secretion of the indolethylamine-N-methyltransferase (INMT) in BL21(DE3)pLysS E. coli. The INMT enzyme is cloned into a high-copy vector with key features that allow 1) tight induction by the lactose analog, β-D-thiogalactoside (IPTG) 2) an N-terminal secretory signal peptide [MKKTAIAIAVALAGFATVAQA (SEQ ID NO:574)] and 3) C-terminal fusion to a HIS tag for purification. E. coli cells harboring the INMT-expression vector are grown in M9 minimal media with 1% glucose for 18 h at 37° C. and shaking at 300 rpm. Concentrated cell culture is diluted to an OD₆₀₀ =1 in fresh M9 minimal media with 1% glucose and 0.2 mM IPTG. After a 3h induction at 18° C. and 300 rpm shaking, tryptamine is added to the media at a final concentration of 1-2 mM and SAMe is added to the media at a final concentration of 1-2 mM. Cells are grown at room temperature for 48 h, shaking at 300 rpm. Media is collected at 24 h and 48h and analyzed by HPLC as described herein.

Alternatively, we describe a strategy for production of the compounds, NMT, DMT, and TMT by recombinant expression and secretion of the indolethylamine-N-methyltransferase (INMT) in Saccharomyces cerevisiae. The INMT enzyme is cloned into a high-copy vector with key features that allow 1) tight induction by the sugar, galactose 2) an N-terminal alpha factor secretion leader sequence, [MEGVSLEKREAEA (SEQ ID NO:574)] and 3) c-terminal fusion to a HIS tag for purification. Saccharomyces cerevisiae cells harboring the INMT-expression vector are grown in CM minimal media with 2% glucose for 18 h at 30° C. and shaking at 300 rpm. Concentrated cell culture is diluted to an OD₆₀₀ =1 in fresh CM minimal media with 2% galactose. After 24h of induction at 30° C. and 300 rpm shaking, tryptamine is added to the media at a final concentration of 1-2 mM and SAMe is added to the media at a final concentration of 1-2 mM. Cells are grown at 30° C. and 300 rpm shaking for 48 h. Media is collected at 24h and 48h and analyzed by HPLC as described herein.

Alternatively, we describe a strategy for production of the compounds, NMT, DMT, and TMT by recombinant expression and secretion of the indolethylamine-N-methyltransferase (INMT) in Komagataella phaffii. The INMT enzyme is cloned into a high-copy vector with key features that allow 1) induction by methanol with the AOX1 promoter and 2) a secretion signal consisting of the α-factor pro region. K. phaffii cells harboring the DAC enzyme are inoculated into 5 mL of YPD in a 15-mL culture tube. After a day of incubation at 30° C. with shaking at 220 rpm, an aliquot of the culture is diluted to an OD₆₀₀=0.2 in 5 mL of BMG (buffered minimal glycerol media) in a 15-mL culture tube. This tube is incubated under the same conditions as before. The following day, the culture is centrifuged at 3000 rpm (2000×g) for 5 min and resuspended in 25 mL BMM (buffered minimal methanol media) to attain an OD₆₀₀ = 1.0. 25 mL of this culture is placed in a 250-mL baffled flask, and during this induction phase, the cells are incubated at 25° C. with shaking at 150 rpm to reduce loss of methanol. After 1 day of induction, an additional dose of 125 µL methanol is added (yielding a final concentration of 0.5%), tryptamine is added to the media at a final concentration of 1-2 mM, SAMe is added to the media at a final concentration of 1-2 mM, and the incubation is continued for another day. After 48 h of induction, media is collected at at 24 h and 48 h and analyzed by HPLC as described herein.

### Example 21 - Conversion of Serotonin to 5-OH-NMT, 5-OH-DMT, and 5-OH-TMT Using a Bio-Based Enzyme Factory

Herein we describe a strategy for 5-OH-NMT, 5-OH-DMT, and 5-OH-TMT production by recombinant expression and secretion of the indolethylamine-N-methyltransferase (INMT) in BL21(DE3)pLysS E. coli. The INMT enzyme is cloned into a high-copy vector with key features that allow 1) tight induction by the lactose analog, β-D-thiogalactoside (IPTG) 2) an N-terminal secretory signal peptide [MKKTAIAIAVALAGFATVAQA (SEQ ID NO:574)] and 3) C-terminal fusion to a HIS tag for purification. E. coli cells harboring the INMT-expression vector are grown in M9 minimal media with 1% glucose for 18 h at 37° C. and shaking at 300 rpm. Concentrated cell culture is diluted to an OD₆₀₀ =1 in fresh M9 minimal media with 1% glucose and 0.2 mM IPTG. After a 3 h induction at 18° C. and 300 rpm shaking, serotonin is added to the media at a final concentration of 5 mM and SAMe is added to the media at a final concentration of 1-2 mM. Cells are grown at room temperature for 48 h, shaking at 300 rpm. Media is collected at 24 h and 48 h and analyzed by HPLC as described herein.

Alternatively, we describe a strategy for production of the compounds, 5-OH-NMT, 5-OH-DMT, and 5-OH-TMT by recombinant expression and secretion of the indolethylamine-N-methyltransferase (INMT) in Saccharomyces cerevisiae. The INMT enzyme is cloned into a high-copy vector with key features that allow 1) tight induction by the sugar, galactose 2) an N-terminal alpha factor secretion leader sequence, [MEGVSLEKREAEA (SEQ ID NO:574)] and 3) c-terminal fusion to a HIS tag for purification. Saccharomyces cerevisiae cells harboring the INMT-expression vector are grown in CM minimal media with 2% glucose for 18h at 30° C. and shaking at 300 rpm. Concentrated cell culture is diluted to an OD₆₀₀ =1 in fresh CM minimal media with 2% galactose. After 24 h of induction at 30° C. and 300 rpm shaking, serotonin is added to the media at a final concentration of 5 mM and SAMe is added to the media at a final concentration of 1-2 mM. Cells are grown at 30° C. and 300 rpm shaking for 48 h. Media is collected at 24 h and 48 h and analyzed by HPLC as described herein.

Alternatively, we describe a strategy for production of the compounds, 5-OH-NMT, 5-OH-DMT, and 5-OH-TMT by recombinant expression and secretion of the indolethylamine-N-methyltransferase (INMT) in Komagataella phaffii. The INMT enzyme is cloned into a high-copy vector with key features that allow 1) induction by methanol with the AOX1 promoter and 2) a secretion signal consisting of the α-factor pro region. K. phaffii cells harboring the DAC enzyme are inoculated into 5 mL of YPD in a 15-mL culture tube. After a day of incubation at 30° C. with shaking at 220 rpm, an aliquot of the culture is diluted to an OD₆₀₀=0.2 in 5 mL of BMG (buffered minimal glycerol media) in a 15-mL culture tube. This tube is incubated under the same conditions as before. The following day, the culture is centrifuged at 3000 rpm (2000×g) for 5 min and resuspended in 25 mL BMM (buffered minimal methanol media) to attain an OD₆₀₀ = 1.0. 25 mL of this culture is placed in a 250-mL baffled flask, and during this induction phase, the cells are incubated at 25° C. with shaking at 150 rpm to reduce loss of methanol. After 1 day of induction, an additional dose of 125 µl methanol is added (yielding a final concentration of 0.5%), serotonin is added to the media at a final concentration of 5 mM, SAMe is added to the media at a final concentration of 1-2 mM and the incubation is continued for another day. After 48 h of induction, media is collected at 24 h and 48 h and analyzed by HPLC as herein.

### Example 22 - Purification of Recombinant INMT Enzyme to Use for in vitro Reactions

The INMT enzyme is cloned into a high-copy vector with key features that allow 1) tight induction by the lactose analog, β-D-thiogalactoside (IPTG) 2) an N-terminal secretory signal peptide [MKKTAIAIAVALAGFATVAQA (SEQ ID NO:574] and 3) C-terminal fusion to a HIS tag for purification. E. coli cells harboring the INMT-expression vector are grown in M9 minimal media with 1% glucose for 18 h at 37° C. and shaking at 300 rpm. Concentrated cell culture is diluted to an OD₆₀₀ =1 in fresh M9 minimal media with 1% glucose and 0.2 mM IPTG and grown for 48 h.

The supernatant containing the recombinant proteins is equilibrated in binding buffer (50 mM sodium phosphate, 0.5 M NaCl, 20 mM imidazole, 10% glycerol, 10 mM 2-mercaptoethanol, 1 mM PMSF, Complete EDTA-free (1 tablet/100 ml), 20 mM 1-phenyl-2- thio-urea; pH 7.4) and centrifuged at 2,500 g for 5 min to remove insoluble matter. Then the supernatant is filtered through a 0.45 µm filter (Millipore, MA, USA) and applied onto a HisTrap HP column (GE Healthcare Bioscience). The recombinant proteins are eluted with a step gradient of imidazole (concentrations of 5, 20, 40 and 300 mM). Fractions are analyzed by SDS-PAGE and stored at -80° C. before use.

### Example 23 - In Vitro Reactions With Purified INMT Enzyme or INMT Lysate

Purified INMT protein is resuspended in activity buffer [100 mM sodium phosphate buffer, pH 6.55, PMSF (1 mM), EDTA-free protease inhibitor] cocktail at working concentration (Roche, Meylan, France) for use in in vitro assays. 0.1 mg/mL of INMT protein is added to a tube with a final volume of 600 uL per sample and added to 100 mM sodium phosphate buffer (pH 7.5), 2 mM tryptamine, serotonin, or melatonin, 2 mM S-adenosylmethionine, and 5 mM MgCl₂.

Alternatively, 0.1 mg/mL BSA protein-equivalent of INMT lysate is used in the same reaction. INMT lysate is derived from E. coli cells harboring the INMT-expression vector. They are grown in M9 minimal media with 1% glucose for 18 h at 37° C. and shaking at 300 rpm. Concentrated cell culture is diluted to an OD₆₀₀ =1 in fresh M9 minimal media with 1% glucose and 0.2 mM IPTG and grown for 48 h. Cell pellets are resuspended in 100 mM sodium phosphate buffer at pH 7.5 and lysed using sonication. After lysis, samples are pelleted by centrifugation (16,000 g, 4° C., 20 min) and supernatant containing INMT is harvested.

### Example 24 - Method of Growth

Modified host cells that yield substituted indoles and tryptamine compounds, such as the DMTP-producing strain herein, express engineered DMTP biosynthesis genes and enzymes. More specifically, the DMTP-producing strain herein is grown in a minimal, complete culture media containing yeast nitrogen base, amino acids, vitamins, ammonium sulfate, and a carbon source of glucose and galactose. The recombinant host cells are grown in 24-well plates or shake flasks in a volume range of 2 mL to 100 mL of media starting from an inoculation density of OD600nm=1. Exogenous L-tryptophan and L-methionine up to 1% can be added to media to supplement the precursor pool for DMTP production. Exogenous L-tryptophan can be taken up by strains expressing the TAT2 L-tryptophan importer protein. Exogenous L-methionine can be taken up by strains expressing the MUP1 L-methionine permease protein. The strains herein can be harvested during a fermentation period ranging from 12 hours onward from the start of pathway enzyme induction.

### Example 25 - Detection of Isolated Product

To identify fermentation-derived tryptamine, DMTP, NMT, DMT, and all other products of a recombinant host expressing an engineered biosynthetic pathway for substituted indoles (see FIG. 11), an Agilent 1100 series liquid chromatography (LC) system equipped with a HILIC column (Primesep 100, SIELC, Wheeling, IL USA) is used. A gradient is used of mobile phase A (ultraviolet (UV) grade H₂O+0.2% TFA) and mobile phase B (UV grade acetonitrile+0.2% TFA). Column temperature is set at 40° C. Compound absorbance is measured at 270 nm using a diode array detector (DAD) and spectral analysis from 200 nm to 400 nm wavelengths. A secondary wavelength of 315 nm is used to selectively detect 4-hydroxy and 4-methoxy substituted indoles. A 0.1 milligram (mg)/milliliter (mL) analytical standard is made from certified reference material for each of the substituted indoles (Cayman Chemical Company, USA). Each sample is prepared by diluting fermentation biomass from a recombinant host expressing the engineered biosynthesis pathway 1: 1 in 100% ethanol and filtered in 0.2 um nanofilter vials. The retention time and UV-visible absorption spectrum (i.e., spectral fingerprints) of the samples are compared to the analytical standard retention time and UV-visible spectra (i.e. spectral fingerprint) when identifying the substituted indole compounds. For example, FIG. 17 depicts the detection of tryptamine isolated from a fermentation with a recombinant host expressing enzymes for L-tryptophan to tryptamine conversion. Detection and isolation is depicted by retention time matching of fermentation derived tryptamine with a tryptamine analytical standard, along with a matching UV-vis spectral fingerprint (i.e. spectral fingerprint) of the fermentation derived tryptamine with the tryptamine analytical standard. This also corroborates that the recombinant host is able to successfully convert L-tryptophan to tryptamine, which further validates that the systems and methods herein direct molecules into tryptamine pathways.

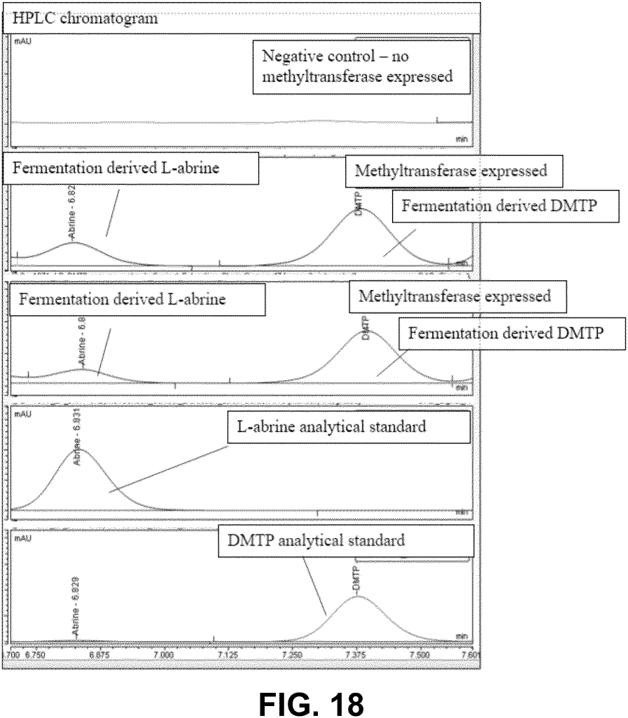

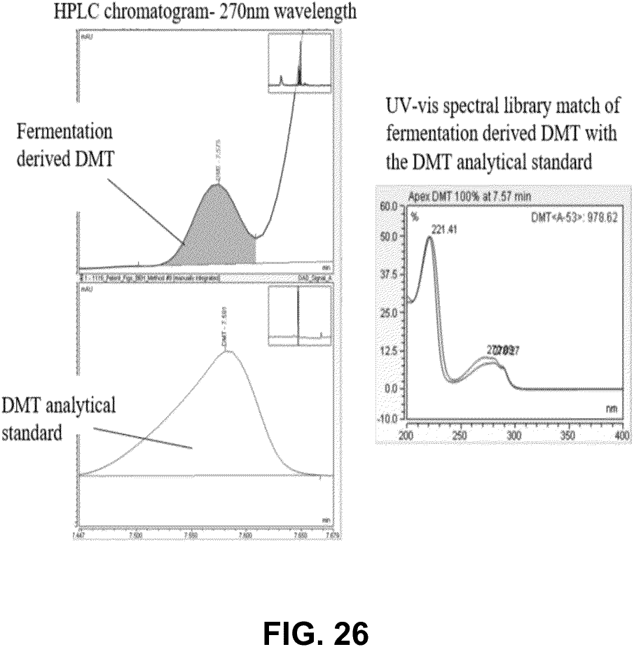

As another example, FIG. 18 depicts the production, detection, and isolation of the substituted indole, DMTP, from a fermentation of a modified recombinant host expressing the DMTP pathway. The retention time and UV-vis spectral absorption (i.e. spectral fingerprint) of the DMTP isolated from fermentation is identical to the retention time and UV-vis spectral absorption (i.e. spectral fingerprint) of the DMTP analytical standard. FIG. 18 also depicts a negative control fermentation from a host strain not expressing the TrpM enzyme or the DMTP pathway, and this strain does not produce DMTP. The modified host strain expressing the TrpM and DMTP producing pathway, highlighted in FIG. 18, is able to produce DMTP. FIG. 26 depicts the production, isolation, and identification of the dimethylated tryptamine, DMT, derived from a fermentation of a recombinant host expressing the pathway for substituted indoles and tryptamines. The fermentation derived DMT is identified by matching retention times with the DMT analytical standard. Spectral library identification of the fermentation derived DMT matches the UV-vis absorption spectrum (i.e. spectral fingerprint) of the DMT analytical standard.

### Example 26 - Synthetic Preparation of Substituted Indoles From Recombinant Host Products

In some instances, it may be preferable, for reasons of either cost or product quality, to utilize recombinant host pathways to accomplish the first part of a substituted indole synthesis and complete the remaining steps synthetically. The tryptamine, as obtained from the recombinant organism, is of a particular grade such that methylations with robust methylating agents selectively leads to mono- or di-methylation. One of ordinary skill in the art would appreciate this as improvement when a primary amine subjected to robust methylating agents as a mixture of alkylation products are not obtained, while obviating the need for tedious chromatography.

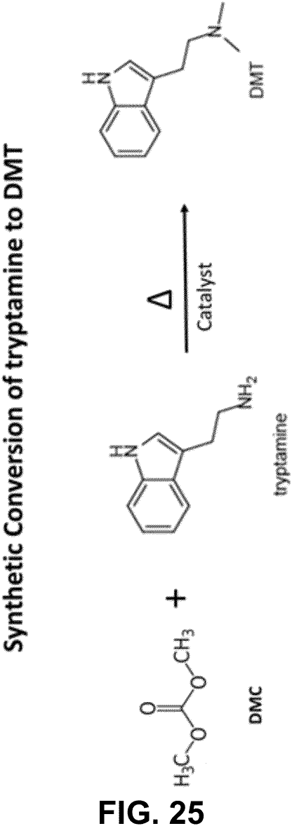

One example would be the production of tryptamine via fermentation of a recombinant host organism, followed by N,N-methylation via methylation chemistry to yield DMT. In one embodiment, the reaction of tryptamine would proceed with a 30-fold molar excess of dimethyl carbonate (DMC) under an inert atmosphere, utilizing a Y-type zeolite catalyst (see FIG. 25. This reaction is carried out at 190° C. for 6 hours in a pressurized reactor vessel or autoclave; another embodiment would utilize a microwave oven for 15-60 minutes. The DMT product is recovered from the volatile DMC reactant via distillation. Another embodiment of the combined biosynthetic and chemical synthesis route is the production of tryptamine via recombinant host organism, followed by its reaction with DMC in the presence of the catalyst: 1,8-Diazabicyclo[5.4.0]undec-7-one (DBU). This catalyst can be used in a thermally heated reactor system at 90° C. for 6-24 hours or used in a pressurized microwave reactor system for less than one hour. Another embodiment of the combined biosynthetic and chemical synthesis route is the production of tryptamine via recombinant host organism, followed by its methylation to DMT using dimethylsulfoxide (DMSO). The catalysts for this system is acetic acid, and the reaction is carried out in a thermally heated reactor at 150° C. for 6-15 hours.

### REFERENCES

Bishop et al., 2006, J Virol. 80:8225-8235.

Blei et al., 2018, Chembiochem. 19:2160-2166.

Boyce et al., 2019, Fungal Ecol. 41:147-164.

Buller et al., 2015, Proc Natl Acad Sci U S A. 112:14599-604.

Byeon Y and Back K, 2016, Appl Microbiol Biotechnol. 100:6683-6691.

Fan, Aili, and Shii-Mii,ig Li, 2014, Tetrahedron Letters 55: 5199-5202.

Francis et al, 2017, Chembiochem. 18:382-386.

Fricke et al., 2017, Angewandte Chemie International Edition 56: 12352-12355.

Fujiwara et al., 2010, JBiol Chem. 285:11308-13.

Germann et al., 2016, Biotechnol J. 11:717-24.

Green and Sambrook, 2012, Molecular Cloning: A Laboratory Manual (Fourth Edition), Cold Spring Harbor Laboratory Press, Cold Spring Harbor, N.Y.

Hemmi et al., 2001, Biochemistry 40: 3512-3524.

Horita, A., and L. J. Weber, 1961, Proceedings of the Society for Experimental Biology and Medicine 106: 32-34.

Huang et al., 2018, AJSH 40:1-7.

Kong et al., 2016, Sci Rep. 6:20273.

Lenz, C., et al., 2019, Angewandte Chemie (International ed. in English) 59.4 (2019): 1450-1454.

Levine, WG, 1967, Nature 215: 1292-1293.

Lima da Cruz et al., 2018, Frontiers in molecular neuroscience 11: 312.

Nemoto et al., 1995, European Journal of Biochemistry, 233:1-8.

Keshavareddy et al., 2018, Int. J. Curr. Microbiol. App. Sci. 7:2656-2668.

Ruohonen, et al., 1997, Yeast 13: 337-351.

Winn et al., 2018, Angew Chem Int Ed Engl. 57:6830-6833.

Zehner, et al., 2005, Chemistry & biology 12: 445-452.

Zou et al., 2013, Angew Chem IntEd Engl. 52:12951-5.

U.S. Pat. No. 10,435,727.

U.S. Pat. No. 10,671,632.

U.S. Pat. Application 16/553103.

U.S. Pat. Application 16/553120.

U.S. Pat. Application 17/068636.

U.S. Provisional Pat. Application 63/035692.

U.S. Provisional Pat. Application 63/053539.

U.S. Provisional Pat. Application 63/141486.

U.S. Provisional Pat. Application 63/164126.

U.S. Pat.Application Publication 2020/0063170.

U.S. Pat. Application Publication 2020/0063171.

U.S. Pat. Application Publication 2021/0147888.

U.S. Pat. Application Publication 20210108238A1.

Finnish Patent Application Publication FI20185254A1.

http://parts.igem.org/Yeast.

In view of the above, it will be seen that several objectives of the invention are achieved and other advantages attained.

As various changes could be made in the above methods and compositions without departing from the scope of the invention, it is intended that all matter contained in the above description and shown in the accompanying drawings shall be interpreted as illustrative and not in a limiting sense.

All references cited in this specification, including but not limited to patent publications and non-patent literature, and references cited therein, are hereby incorporated by reference. The discussion of the references herein is intended merely to summarize the assertions made by the authors and no admission is made that any reference constitutes prior art. Applicants reserve the right to challenge the accuracy and pertinence of the cited references.

As used herein, in particular embodiments, the terms “about” or “approximately” when preceding a numerical value indicates the value plus or minus a range of 10%. Where a range of values is provided, it is understood that each intervening value, to the tenth of the unit of the lower limit unless the context clearly dictates otherwise, between the upper and lower limit of that range and any other stated or intervening value in that stated range is encompassed within the disclosure.

That the upper and lower limits of these smaller ranges can independently be included in the smaller ranges is also encompassed within the disclosure, subject to any specifically excluded limit in the stated range. Where the stated range includes one or both of the limits, ranges excluding either or both of those included limits are also included in the disclosure.

The indefinite articles “a” and “an,” as used herein in the specification and in the embodiments, unless clearly indicated to the contrary, should be understood to mean “at least one.”

The phrase “and/or,” as used herein in the specification and in the embodiments, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements can optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the embodiments, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the embodiments, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the embodiments, shall have its ordinary meaning as used in the field of patent law.

As used herein in the specification and in the embodiments, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements can optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

### SEQUENCE LISTING

Seq. ID No: 1

>6xHISCACCACCACCACCATCAC

Seq. ID No: 2

>AADC_1ATGGAAACGGTGAATAAATCTTGTTGCTCCTTAGCGACACCGC
ATCTTCCTCTTATGTCACCGCACCTACTACACTCTCACAGGGACTCTCCC
GTTCTTAAAATAATCCACTCTATTGTGCTGACTGTGCAGAATAACCATTC
TTGCTTGCAGGCGTTTCTTGGGATTGGGACAGATAATGTAATCCTAGTAA
AGACTAATGATAGGGGTAAAATGATACCAGAAGACCTGGACCATAAGATC
CAGAAAGTCAAGTCCGAAGGTTCAGTTCCATTTCTGGTTTCAACTACCTG
TGGGACAACCGTGTTCGGCGCGTTTGATCCATTAGAGGGCATTGCAGATG
TTTGCGAACGTCATTCCCTGTGGCTGCATGTTGACGCTGCATGGGGAGGT
TCAGCCCTTTTAAGCAGCCGTCACAGACATTTGCTTAAAGGCATCGAGAG
GGCTGATAGTGTGACATGGAATCCTCATAAACTATTGGGTGTTGGACTGC
AGTGTTCCGCCTTTTTGCTGCGTGATACGACTCAGTTACTAGAGAGGTGT
CACGCTGCAAACGCAACGTATTTATTCCAGACCGATAAATTCTATAACCT
GCAGTATGATACTGGTGATAAAAGCATTCAATGTGGAAGAAGGGTAGATT
GCTTAAAGCTGTGGCTAATGTGGAAAGCCTTGGGATCGAAGGGTTTGGAA
ACCAGAGTCGATAGAGTATTAGACCACACTAGGTACCTTGTAGAGGAAAT
GAAGAGGAGGGAGGGATTTAGACTGATAATGGAGCCAGAATTTGTCAATT
TATGCTTTTGGTACGTGCCTCCCTCTCTTAGGAACAAAGAGAATAGTCCA
GATTTTTGGACCAGGTTGGGTAAAGTCGCCCCAGTCATAAAAGAGCGTAT
GATGAAGAAAGGGTCAATGATGGTGGGCTACCAGCCTCACGGGAACATGG
TTAACTTTTTCAGGCAGATAGTCGTGAATCCAGAAGTCACCAAAGAGGAC
CTAGATTTCTTTCTTGATGAGATAGAGCGTTTAGCAGAGGACCTT

Seq. ID No: 3

>AADC_2ATGTGGGGTTGCGGGAACGGTGATTGTATCCACGTTTTACTGC
TTATCAGCCATACGAGTCCACCACCCCTTAGTCCGCATCTGTTGCACTCC
CACAGGGACTCCCCGGTTCTGAAAATCATCCACAGTATCGTCCTTACAGT
CCAGAACAATCATTCCTGCCTACAGGGCCATGTGCCATTTTATGTTAGTG
CCACTGCGGGAACAACGGTGTATGGAGCTTTCGATCCTTTCGTCAAGATT
GCGGATATATGCCAGAAACACGGACTTTGGATGCTGCACGTAGACGCGGC
TTGGGGTGGGGGTTTGCTTATGTCTCGAAAACACAGGCACAAGATGAACG
GGATAGAAAGGGCGGACAGCGTGACTTGGAACCCACATAAGATGATGGGA
GTCCTTTTGCAATGTAGTGCAATCCTTCTGAAGGAAAAGGGTATACTACA
GGGTTGTAATCAAATGTGCGCAGGGTACTTATTTCAGCAGGACAAGCAAT
ATGATATTTCTTACGATACCGGCGACAAAGCCATCCAGTGTGGCAGACAC
GTTGATATTTTCAAATTTTGGCTGATGTGGAAAGCAAAAGGGACAGTTGG
CTTCGAGCAACAAATTAACAAGTGCTTAGAGTTGTCCGAGTACCTATACT
CTAAGATTTGCAACCGTGAAGATTTTGAGATGGTTTTTAAGGGAGAAGTG
TCTTTACACCGTTTGGAAGAGGGAATTAAG

Seq. ID No: 4

>AADC_3ATGGAAACAGTGAACAAATCTTGTTGTTCATTAGCAACGCCGC
ACCTTCCTCTTATGAGTCCACATTTGCTACACTCTCATAGAGACTCCCCA
GTTCTTAAAATTATACATTCTATCGTCTTGACAGTGCAAAACAACCATTC
CTGCTTACAAGGTCACGTCCCGTTCTATGTGAGCGCCACGGCGGGCACCA
CAGTTTACGGAGCCTTTGATCCATTCGTTAAGATCGCTGACATATGCCAG
AAGCACGGCTTGTGGATGCATGTAGATGCAGCATGGGGAGGCGGTCTTTT
ACTGAGCAAAAAGCACAGGACGAAATTAAGTGGGATCGAGAGGGCAAATA
GTGTGACTTGGAATCCTCACAAGATGATGGGGGTGCCACTTGTAAAAGAT
AACATGGACCTTCTGAAGAGATGCCACTCCGCCGAAGCCAGTTACTTGTT
TCAGCAGGATAAGTTCTATGACGTAAGATACGACACGGGAGACAAATCCA
TCCAGTGTAGTAGGCGTGCGGATGCATTCAAGTTTTGGATGATGTGGAAG
GCGCTGGGGACACTTGGGCTAGAGGAAAGGGTTAATCGTGCCCTGGCCCT
GTCTAAGTACCTAGCCAAGGAGATCAAGAAAAGAGACGGATTTGAACTGA
TTTGGGAGCCAGAATATGCTAACATCTGTTTTTGGTACATCCCACCGAGT
TTACGTAAGATGGAAAATAAAGGTCCTGAGTACTGGCAAAGGTTAAACCA
GGTCGCACCAACAATAAAAGAGCGTATGATGAAGAAGGGTTCCATGATGA
TTGGATATCAGCCCCACCGTGACAAGGTGAACTTTTTCCGTCACATCATT
ATTTCACCGCAGGTCAGTCGTGAGGATATGGACTTTGTCTTAGATGAGAT
TGACTTACTTGGCCGTGACTTA

Seq. ID No: 5

>AADC_4ATGAATGCCTCAGAATTTCGTAGGAGGGGGAAGGAGATGGTCG
ATTACGTAGCAAATTACATGGAAGGGATTGAGGGGCGTCAGGTATACCCA
GACGTGGAACCTGGGTATTTAAGGCCGCTTATTCCAGCTGCAGCCCCACA
GGAGCCCGATACCTTCGAAGACATCATAAATGACGTTGAGAAAATCATAA
TGCCTGGCGTCACTCATTGGCATTCTCCATACTTTTTCGCCTACTTTCCC
ACTGCTTCTTCCTACCCAGCCATGCTTGCAGACATGTTATGTGGCGCAAT
AGGATGTATTGGGTTCTCCTGGGCGGCATCCCCCGCCTGCACGGAGCTAG
AGACAGTTATGATGGATTGGTTGGGTAAAATGCTTGAGCTGCCCAAAGCG
TTCTTGAACGAGAAGGCAGGAGAAGGGGGTGGCGTGATCCAAGGCTCTGC
TAGTGAAGCCACCCTAGTCGCCTTGCTGGCAGCGAGAACTAAGGTGATAC
ACAGACTTCAAGCCGCCTCTCCCGAGTTAACTCAGGCTGCAATTATGGAG
AAATTGGTAGCCTATTCTAGCGACCAGGCTCACTCCAGCGTAGAACGTGC
CGGTCTGATTGGGGGCGTGAAGTTGAAAGCTATTCCGAGCGATGGGAATT
TTGCCATGAGAGCGTCCGCGCTTCAAGAGGCCTTGGAACGTGACAAGGCT
GCCGGATTAATCCCGTTCTTTATGGTCGCTACGCTTGGTACTACCACGTG
TTGTTCTTTTGATAACCTACTGGAAGTGGGTCCTATCTGCAATAAGGAGG
ACATTTGGCTTCATGTAGACGCTGCCTATGCAGGTTCTGCTTTCATTTGT
CCCGAATTTAGGCACCTGCTTAATGGCGTGGAGTTCGCGGATAGTTTTAA
TTTTAACCCTCATAAATGGTTGCTGGTGAATTTTGACTGTAGCGCCATGT
GGGTCAAGAAAAGAACGGATCTAACTGGAGCCTTTAGACTTGACCCCACG
TACCTTAAACACAGCCATCAGGATTCCGGGCTAATAACCGATTATCGTCA
CTGGCAAATCCCGTTGGGAAGACGTTTTCGTAGCCTGAAGATGTGGTTTG
TGTTTAGAATGTATGGGGTGAAAGGGCTACAGGCGTACATACGTAAGCAT
GTCCAACTTTCTCACGAGTTTGAGTCTCTTGTACGTCAAGACCCGAGATT
TGAGATTTGCGTTGAGGTAATACTAGGTCTAGTCTGTTTCAGGCTTAAAG
GCTCAAACAAGGTCAACGAAGCGTTATTGCAAAGAATAAATAGTGCTAAG
AAGATCCACCTTGTTCCCTGCCATCTACGTGATAAGTTTGTCTTACGTTT
TGCGATATGTAGTAGGACGGTAGAAAGCGCACACGTTCAGCGTGCGTGGG
AGCACATAAAGGAGTTAGCCGCTGATGTGTTGAGAGCTGAACGTGAG

Seq. ID No: 6

>AADC_5ATGGCCAGTGGGTATCCAGGGGCGGGAGCCCAACAGCCGCCTG
CGGCTCCTGCCAGTGGATCAGGTTCCCCCGTCTCCATGCCGTACTACGCT
TCCGAATTAGCAAGGGCGAAGCACGAGGATGACATGAAAATGCCGGAACA
TGGGATAGAACCTAGGCACTGTCTTAGACGTATCGAGGATTACCATCTAT
TGGACTTCAGTGAAAGGTTAAACACGAGTTCATACGTCAATGTAGTCTTT
GAACCAGAAGAGGAGACGGTTGCTAACATGGGGTTGAAAGTCAATTTAGC
TGATCAAACAGTATATCCCGAAAGTTTCCGTATGCACAATGACACAGTCA
ATATGATCGCTAAGTTATGGAATTGCCCTAAGCCTGCCGACTTTGATGAA
TATGGATGTTATGCCGGAGCAGGCACCGTTGGTTCCACCGAAGCGTGTCT
GCTTGGAGGATTGGCTCTTAAATTCAGATGGCGTAAATGGTACGCCGCCA
AACACGGAATGGATCAGAACAAAGTAAGAGGTGTGTACCCGAACCTTGTT
ATCACTACGATGTTTCAAGCCGCTTGGGAAAAACTTTTCAAATATATGGA
CATTGAACCCAGATTCGTGACCCCATCCTGGAAGACATTCACTATGGACC
CATCCGGCCTTGAAAAAGTGGTAGACGACAAAACTATAGGTGTGGTGTGT
ATTATGGGAAATCACTATGGTGGTCAATACGATCCAGTGTGGGAAGTGAA
CGACGTGCTTGAGAAGATTAACAAGGAAAAGGGACTTCAGGTAGGTATCC
ATGTGGACGGAGCGTCTGGCGGATTCATAGCCCCCTTCCAAGAGGGATTA
CCGGCATGGGATTTTAGACTAAAGAATGTGTTAAGTATAAGCGCGTCTGG
ACACAAATTCGGAAACTCCTGTTGTGGAACAGGATGGATTATTTGGAGAG
AAAGGAAAGGACTTTCTGACACTGTCGCCATCAATGTATCTTACCTTGGA
GGGTCCGCAGACAGCTATACGTTAAACTTCAGCAGACCGGCGCAAGGTGT
ATATGTCCAG

Seq. ID No: 7

>AADC_6ATGGCGGGCAGCATCGACGTGATGCTTAGGGACCTGAATCTTG
ACACCGTTGAATCTCTTCCAGCAGACTTTGATCCGACCACAGTAATAAAT
GATATACTACCACCAGTTGTAAACGACGCCAAACTGGTTAATGGCATTGA
GAAACCCAGAGAAATAGTCCTGGGCAGAAATGTACATACTTCCAGTTTGG
AGGTAACAGAGCCCGACGCCGATGATGAGGTTACCGGGGAGAGAGAGGCG
TACATGGCGTCAGTACTAGCTCGTTATCGTAAGTCTCTGCTAGAGCGTAC
AAAACACCATTTAGGTTACCCATACAATTTGGATTTTGACTATGGGGCTT
TGTCCCAGCTTCAGCACTTTTCAATCAATAATCTTGGAGATCCATTTATC
GAGAGCAACTATGGCGTACATAGCCGTCAGTTTGAGGTAGGGGTCTTGGA
CTGGTTTGCAAGACTATGGGAGCTGGAACGTAACGAATATTGGGGGTATA
TTACGAATTGTGGGACAGAGGGTAACTTGCATGGCATATTAGTCGGCAGG
GAGGTCTTTCCCGACGGTATCTTGTATGCAAGCAGTGAATCCCACTACAG
TATATTCAAAGCCGCGAGGATGTATAGAATGGATTGCGAAAAGGTCAATA
CGCTTATCAGTGGAGAAATCGACTGTGAGGATTTCAAGGCAAAGTTATCT
CTGCACAAGGATAAGCCCGCGATCATAAACGTCAACATTGGGACAACAGT
AAAGGGCGCGGTGGATGATCTGGATCTTGTTATTAAAACCCTGGAGGAAA
GTGGCTTTAGTCACGACCGTTTCTACATCCATTGTGATGGGGCGCTTTTT
GGGCTAATGATGCCTTTCGTAAAGCTGGCACCCAAGGTAAGTTTCAAGAA
ACCCATTGGTAGTGTGAGTGTGTCTGGGCATAAGTTTGTCGGCTGTCCTA
TGCCGTGTGGTGTACAAATTACCAGACTTGAGCATATCAATGCTCTTAGC
CGTAATGTAGAGTACCTGGCGTCTAGGGATGCTACCATTATGGGCTCAAG
AAACGGTCATGCACCCTTGTTTCTTTGGTATACTTTAAATCGTAAAGGTT
ACAGGGGATTCCAGAAAGAGGTACAAAAGTGTCTTCGTAACGCTCATTAT
CTTAAAGGTAGGCTAACTGAAGCAGGGATTGGCGCGATGCTAAACGAGCT
AAGTAGCACTGTGGTTTTTGAACGTCCTCAGGATGAAGAGTTCACTCGTA
AATGGCAATTGGCGTGTCAAGGTAATATTGCGCATGTAGTCGTCATGCCG
AACATTAATATTGATAAGTTGGACCATTTTGTCAATGAATTAGTAGAGAG
AAGGGCTGTATGGTACGAGAATGGGAAGTTGAAGTCTCCATGTGTTGCCT
CCGAAATAGGCAACTCTAACTGCTTATGTGCGTTGCATAAG

Seq. ID No: 8

>AADC_7ATGGTGAGGGCCGTTGAAAAGCCAGTACAAGCCATAGTAAATG
CCGCGTTTAGAGGGAAAGATGCATATCATGTTTTCAGAACAACAGTTCTA
GCGGCCGTGCTGTTAAGACTGTGGAGACACTTGAGACGTGTGATGGCTCA
TGAAGGTTTGAAAGCATACTTTATGTCCTTAGTCGCACCGCATCTGAAGA
AACTTCCGTACGTCCAAAACAAGTTGAAGAAGGAGATGGATAAAACTATG
ACGAAAATGAGAAATACCTTTCGTAAGGAGGTGACAGATCCAAGGACATC
ATTACCTGTTGATGGGATTCCTGAGAAACAGATCTTGGAACTGATTCAAC
ATCGTAAAGAGTTAGATACTAAGGAATGGACAAAGGGTATGACCACTGGC
TGTGTTTACCACGGTGGTCAGGACCATTATGACTTTGTAGGACAGATCTT
TGCATCATGGGGGTTCGCTAATCCATTGCACCCCACAACCTTTCCAAGCC
TACGTCAGATGGATAGCGAGGTCGTACAGATGGTTATCAACATGTACCAC
GGGGATTCAGAGTGCTGCGGGGCGTTCACTACTGGAGGAACGGAGAGCAT
TCTGATGGCGATGAAGGCCTATCGTGATTGGGGTAAGGCAGAAAAGGGTA
TTACCGATCCCAATATCGTTATCTGTAACACCGCCCATGCTGCTTTTGAT
AAGGCCGGCAAGTACTTTAACATCTTCGTAAAACACGCGAGAACGAACTC
TGAAATGGAGATTGATTTGGGCCATCTACGTAGCCTTATCGACTCCAATA
CCGTGGCCATCGTAGGCTCAGCCTGCCAATTTTCTCATGGAACCGTGGAC
CCGATCCAGGAAATGGCAAAGATTGCCATGAAGCGTAGAGTTGGCCTACA
CGTTGATTGTTGCCTAGGCGGTTTTCTAGTTCCGTTCATGGAAAAAGCTG
GATTCCAACTTCCGCCCTTTGACTTCAGAGTCAAGGGGGTAACGTCCATA
TCCTGTGATCCCCACAAATATGGGTTTGCCCCAAAGGGCTCCTCTGTTGT
AATGTTTAGTAACCGTCACCTGAGGCACTACATGTATTGTTTCTTAACAG
AATGGAGCGGAGGAATATACGCGACTGCAACGATGACTGGTTCCAGAGCA
GGTGGCCCGGTGGCCGCCACATGGGCGAGCATGTGCAAATTTGGGGAGAA
AGGCTATATTGAGACAACTAAACAGATCGTCGGGGCCACTAAGAAAATTG
CGGCTGGTATAGCAGAAATCGAAGGTTTACGTGTGGTTGGCAGACCGGAT
GTATGTGTCGTGGCCTTCACCTGTACTGAGGGTTCCGGAATGAATTGCTA
CGCCGTTGGGGACTGTATGCATCAGGACTTTCATTGGGAGTTGCAATCTT
GTCAGAATCCGGCTTGTGTTCACTTGGCTTTAACCCTGCCGACCAGTAGA
AATGCTGATAAATTCGTAGCTGATTTGCGTCAGGCTGTGGAGGCGGTGAG
GAGCGATAAGGACGGCAAATTCGCATCCACGGCGGGCATGTATGGGACCG
CAGCTTCTTTGCCAGCCGCGTTTTTCGAAGACGGCGCAGCAGCATACCTT
GATGCGATGTGTGAAGCCATCCCAGCGGGAGACGCTCTACTGCCGGAGGA
ACCTGCCACGAAGGAATCCCCTGCAGCTGCCGGGGCACCGGCTCAAGCAA
CAGGGGGTGCT

Seq. ID No: 9

>AADC_8ATGAATGCGAGCGAGTTTAGGCGTCGTGGGAAGGAAATGGTTG
ACTACGTAATCACGAATTATCTGGAACAGATAGAGTTACGTCAGGTCTAC
CCTTCCGTCGAGCCGGGGTATCTTAGACCTATGATTCCTGATTCCGCGCC
GGAAGAAGGTGAGACGTATGAAGATATCATGAAAGACATCGAGAGAGTCA
TAATGCCGGGAGTTACTCATTGGAATAGCCCGTACTTCTTTGCGTACTTC
CCTGCGGCAACAAGTTACCCTGCTATGTTGGCCGACATGTTATGCGGCAG
CCTGGGGTGCATCGGGTTCTCATGGGCCGCTTCACCCGCCTGCACAGAGT
TGGAAACCGTAATGCTTGACTGGTTGGGCAAAACAATCGGCCTACCAGAA
CAGTTTCTGGCTGGTACCAACGGAGAGGGTGGCGGTGTAATCCAAGGTAC
TGCCAGCGAAGCGACCTTGATGGCACTATTGGCGGCCCGTACTAAGGTTA
CAAGAAGGTTACAAGCTGAGAACCCTGACCTTTCTGAAGCAGAGATCATT
AGCCGTATGGTGGCATATTCTAGCGACCAGGCCCATTCCAGCGTGGAGAG
AGCTGGGCTGATCAGTGGCGTTAGAATGAAAAAGATCCCATCTGATGAAA
ACTTCACTGCTAGAGGTGAAGCACTAAAGAAAGCACTTGAGGAGGACAAA
GCTGAAGGTTTCATACCAGTCTTTCTTTGCGCGACACTGGGCACAACAAC
TTCTTGCGCGTTCGACAATCTTATGGAACTGGGTCCGATATGTAATGCGG
AGAACATGTGGTTGCATATCGACGCCGCCTATGCTGGGAGTGCTTTCATC
TGCCCGGAGAACAGATATTTAATGAAGGGTGTCGAGTTCGCGGACTCCTT
CAATTTCAACCCACATAAGTGGTTGTTAGTCAACTTCGACTGCAGTGCTT
TCTGGGTAAAGAAAAGGTCCGATCTTATATGTGCATTTAAGATAGACCCC
GTCTACTTACAACACGACCAGCAGGAGAGCGGTTTAGTAACGGATTACAG
ACACTGGCAGATTCCTCTAGGAAGAAGGTTCCGTAGTCTGAAACTGTGGT
TTGTTCTGAGGATGTATGGCGTAAAGGGACTTCAAGCTCATATACGTAAG
CATATTAGACTAGCACAGGAATTTCACGAATTTGTTAAGAACGATGATCG
TTTCGAGATATGTGCACCCGTCATATTGGGTCTGGTGTGTTTTTGCTTGA
AGGGGTCAAATACGCTAAATAAGTCCTTGCTTCAGAAAATTAACACTTTG
AAGAAAATACATCTTGTACCTTCTTGTCTAGGCGATAAATTCATCTTGAG
ATTCGCGGTTTGCGCTAGAACACTAGAGTCAAACCACATTGTGTTCGCCT
GGAAGCACATTGAGGAATTGGCGACTGAGGTTTTAAAGGAGGGAGAGAAA
CAA

Seq. ID No: 10

>AADC_9ATGCAGAACTGCAATCAGATGCACGCCAGTTATCTTTTCCAAC
AAGACAAGCATTATGATCTGTCCTATGATACGGGTGATAAGGCGCTACAG
TGCGGGAGGCATGTGGATATCTTCAAACTTTGGTTAATGTGGCGTGCCAA
GGGCACGACGGGTTTTGAGGTCCAGATAGACAAATGCTTGGAACTTGCTG
AGTATTTGTATGACAAAATAAAGAACCGTGAGGGTTATGAGCTAGTGATA
GAGGGGAAGCCCCAACATACTAACGTCTGTTTCTGGTACATACCCCCATC
ACTTAGACACATGGAGGATAATGAGGAAAGGATGGCTCGTTTGGTTAAAG
TCGCTCCAGTAATAAAGGCTCGTATGATGGAATACGGAACTACAATGGTT
AGCTACCAACCGCTGGGTGACAAGGTGAATTTCTTTCGTATGGTTATATC
CAATCCAGCCGCAACCCATCAAGATATAGATTTTCTAATTGATGAAATTG
AGCGTTTAGGTCAAGACCTG

Seq. ID No: 11

>AADC_10ATGTGGGGATGCGGTAACGGAGATTGCATACACGTTTTGTTA
TTGATCTCCCACACGTCTCCACCTCCCCTGTCACCCCATCTACTACATTC
TCACCGTGATAGCCCGGTATTGAAGATAATCCACAGTATCGTACTGACGG
TGCAGAATAACCATTCTTGCTTACAGGGGCATGTCCCTTTCTACGTGTCC
GCAACTGCAGGGACAACTGTCTACGGCGCATTTGATCCCTTCGTTAAAAT
CGCTGACATTTGTCAGAAGCACGGGTTATGGATGCACGTAGACGCGGCGT
GGGGAGGAGGCTTGCTTCTTAGCAAAAAGCACAGGACTAAACTGTCAGGT
ATAGAGCGTGCCAATTCCGTAACATGGAACCCACATAAGATGATGGGAGT
CCCTCTTTTCCAGTGCTCCGCCTTTTTATTACGTGATACGACTCAACTGC
TAGAGCGTTGTCATGCTGCGAACGCAACATACCTTTTCCAAACCGATAAA
TTCTACAATCTACAATACGATACGGGTGACAAAAGCATCCAGTGCGGGAG
AAGAGTGGACTGTCTGAAACTTTGGTTAATGTGGAAGGCCTTGGGCTCCA
AAGGTCTTGAGAGGAGAGTTGACAGAGTCTTAGACCATACCAGATACCTG
GTCGAGGAGATGAAAAACAGAGAGGGCTTTAGGCTAATTATGGAGCCCGA
GTTCGTGAATCTGTGCTTTTGGTACGTTCCCCCGAGCCTGCGTAATAAGG
AGAACTCACCGGACTTTTGGACAAGGCTGGGT

Seq. ID No: 12

>AADC_11ATGGGGTCTTTGGGAACAAATCCGACGAGCTTTAGCGCCTTC
CCTGATGACAAGGCCGCATTTGAACCATTAAACCCAGAAGATGTTAGAGC
TTATCTTCACAAGGCGGTCGATTTTATCTCAGACTACTACACGAACGTTG
AAAGTATGCCTGTTCTGCCGAACGTGAAGCCGGGATATCTGCAAGACGAG
CTGACGGCTTCTCCACCCACTCACAGTGCCCCATTCGACGTTACAATGAA
GGAACTAAGGACCAGCGTTGTACCAGGCATGACACACTGGGCGTCACCAA
ACTTCTTTGCATTTTTCCCATCAACCAATTCAGCGGCGGCAATAGCCGGT
GATCTAATAGCTTCTGCGATGAATACGGTAGGCTTCACATGGCAGGCGAG
TCCCGCAGCAACGGAGATGGAAGTTTTGGCCCTGGACTGGTTAGCACAGC
TACTACATCTTCCGACGACTTTTATGAATAGAACCTCCACTGGCAGGGGT
ACAGGCGGAGGAGTCATATTGGGTACGACGTCAGAGGCCATGTTAGTTAC
GCTTGTCGCAGCTAGGGACGCTGCTCTGAGGAGAAGCGGCAGCGTGGGAG
TGAGCGACATTCCTAGACTGGCAGTATATGCTGCGGATCAAACACATTCC
ACCTTCTTTAAAGCGTGCCGTTTAGCAGGATTTGACCCAGCTAACATTAG
GAGTATCCCCACTGGACCTGAGACTAATTACGGCTTAGACCCCGCTAAAC
TATTGGAAGTGATGCAGGCTGACGCGGATGCGGGGCTAGTCCCGACTTAC
GTATGCGCCACCGTCGGTACTACATCTTCCAATGCTGTGGACCCGGTTGG
CGCGGTTGCCGATGTCGCCGCCATGTTTAATGCCTGGGTACATGTAGATG
CCGCTTACGCCGGCAGCGCGTGTATATGTCCTGAGTTCCGTCACCATCTT
GACGGCGTAGAGAGAGTCGATAGCATCTCAATGTCCCCTCACAAATGGCT
TTTAACCTGCCTAGATTGCACTTGTCTGTATGTTAGGGATGCTCACAGGT
TGAGTGACTCTCTGGAGACCAACCCCGAGTACTTGAAGAACGACGTTACC
GACAGTGGTGAGGTTACCGACCTAAAAGATATGCAGGTTGGGGTTGGTAG
AAGGTTCCGTGGACTGAAGCTGTGGATGGTTATGAGAACCTATGGCACTG
CGAAACTACAGGAGCACATTCGTTCCGACGTCGCAATGGCCAAAATGTTC
GAGGATAGCGTTAGAGCTGATAATCGTTTTGAGGTCGTCGTACCGAGGAA
CTTTGCCCTTGTATGCTTCCGTATTAAGGCTAGAGGAGATATGACTGAGG
AAGACGCGGACGAAGTAAACAGGTTACTGATGGAGAATTTAAACAAGACG
GGTAAAGCATACTTAGCTCACACTGTGGTCGGGGACCGTTTTGTTTTACG
TTTTGCGGTTGGGAGTAGTCTGCAAGAAGAGAGACATGTGAGGTCAGCCT
GGGATTTGATCAAGAAGACGACTTCATCTATTATGGAT

Seq. ID No: 13

>AADC_12ATGGACCCTCTAAAAGCCGTTGAAATGGTTGACGAAAATACT
ATCTGCGTAGCAGCCATCTTGGGCTCCACTCTGACCGGGGAGTTTGAGAA
CGTAAAATTATTAAATGAGCTATTAACTAAAAAGAACAAGGATACAGGGT
GGGACACGCCTATACACGTTGACGCAGCTTCAGGCGGCTTCATAGCTCCC
TTTCTATACCCGGACCTAGAATGGGATTTTCGTCTTCCCCTTGTGAAGAG
TATCAACGTTTCTGGACACAAATATGGCTTAGTCTACCCCGGCGTGGGAT
GGGTAGTATGGCGTAGCAAAGGGGATCTACCTGACGAACTGATATTTCAT
ATCAATTATCTGGGAAGCGATCAGCCCACTTTTACCTTGAATTTCAGTAA
AGGTAACAATATCAGCACGCACGCTTACAAGAAGCCCAAGTGCCGTTTCT
ACTTCCTAACCAATCAAATCTTATCTAGGCGTTATTTATTACACCGTCTG
AAAAGTGGAACTTCTTTGAATAGTCTTCTA

Seq. ID No: 14

>AADC_13ATGCAGCCAGGGTATTTATCTCGTATGCTGCCTGATTCAGCT
CCCAATCACCCCGAATCTCTTGAGGATATTTTCAATGACATCTCAGCAAA
GATACTTCCCGGCGTCACACATTGGCAGAGCCCGAATTACTTCGCATACT
TCCCCTCTAATTCCAGTATTGCTGGGTTTCTGGGTGAAATGTTGTCCGCT
GGTCTGAACATAGTAGGTTTTTCCTGGATAACTAGCCCAGCGGCTACAGA
ATTAGAAATGATCGTTCTGGACTGGCTTGCGAAATTACTAAAACTTCCTG
ATGACTTTCTTTCCGGTGGCACGTCT

Seq. ID No: 15

>AADC_14ATGGTAGTAGACTACAAGGACTGGCAGATCCCATTAGGACGT
CGTTTTCGTAGCTTAAAATTATGGATGGTACTGAGACTTTACGGAATTGA
AAATTTACAATGCTACATCAGGAATCACATCAAGTTAGCCCAGCAGTTCG
AGGTTCTAGTAGCGCAAGACCTAAGGTTCGAGATCGTATCTCCAAGAATT
TTTAGCTTGGTTTGCTTCAGGTTACTACCTTCCCAAAACTGCAAGGATCA
CGGGAATGAGCTAAACCACCACCTGTTAGATACTGTCAACAGCACGGGGA
AAGTCTTCCTTAGTCATACTGTTCTATCAGGGAAATATATCTTGAGATTC
GCTGTTGGCGCTCCCCTTACGGAGGAAAGGCACGTAACGGCGGCATGGAA
GGTATTACAGGATGAGGCGTCAGCGTTGTTACAGTCACTG

Seq. ID No: 16

>AADC_15ATGGGCTCTCTTGATATAAAGCAGGAATCAAGCCCTCTAATG
ACAAATCCGTTAGATAGCGAGGAGTTCAGGCGTCAAGGATACATGGTGAT
AGACTTTCTAGCCGAGTATTATAAGAACATACAGAAATTTCCGGTTCGTT
CTCAAGTGGAACCTGGGTATTTAAGGAAAAGGCTTCCAGAATCTGCCCCT
TACGAGCCGGAAAGTATAGAGCGTATTCTTAAAGACGTACATGACGACAT
AGTTCCTGGTCTTACTCATTGGCAATCACCGAACTATTACGCATATTTTC
CGTCCAGCGGTAGCACAGCGGGTCTATTGGGTGAAACCCTGGCGGCCGGG
TTCAATGTGGTCGGCTTCAACTGGATCTCCAGTCCTGCATCCACTGAACT
AGAGAGTATCGTAATGGACTGGTTAGCTGAAATGTTGAACCTACCAAAGT
CCTTTACATTTAGCGGAGACGGTGGCGGGGTGATGATGGGTACCACGTGT
GAGGCAATTCTGACAACTATTACAGCTGCACGTGACAGGATACTGGACAG
GATCGGACGTGAGCACATTAATAAGCTGGTCGTTTATGGCTCTGACCAAA
CGCACTGTTCTTTCTTTAAGTCAGCTAAGATCGCTGGTATTCTACCGAAT
AATTTTAGACAAGTTAAAACTAGCAGAGTCAACGCGTTTAGTATGAGACC
CGACGCGCTTCGTGCCGCGATACAGGCGGACGCAGATGCAGGCCTGGTCC
CGTTTTTCTTATGTACCACTGTTGGTACGACCTCAACAGCGGCTGTAGAC
CCGGTAGCGTTACTTTGCGAAGTAACAAAAGACTACGGGATGTGGGTGCA
CATTGATGCAGCGTACGCGGGCAATGCCTGCATTTGTCCAGAGTTCCGTC
ATATGATCAATGGGGTTGAGAATGCCGACTCATTCTCCTTTAACGCGCAT
AAGTGGTTCTTGACTACTCTTGATTGCTGTTGCTTATGGGTTAAAGATCC
AAGCTCACTGGTAAGGTGCCTTAGCACTAATCCCGAATACCTAAAGAATA
AGGCCACTGACACCCAACAAGTTGTTGATTATAAGGATTGGCAAATCACA
CTATCAAGGCGTTTCAGATCTTTA

Seq. ID No: 17

>AADC_16ATGGACGGGCAAATGCTAAAGCCAATGGACGCTGAACAACTG
AGGGAGTATGGTCACCAGATGGTGGACTTTGTAGCGGACTACTACAAAAC
TATTGAGAGCTTCCCCGTGCTAAGCCAAGTTCAACCTGGATACTTACGTG
AGCTAGTCCCTACAAACCCTCCAACGCACCCGGAGTCCCTTCAGGATGTT
CTGGACGATGTTAAAGCGAAGATATTACAAGGCGTAACCCATTGGCAAAG
CCCGGGATATTTCGCCTATTTTCCGTGCAACTCTAGTACCGCGGGATTCT
TAGGAGAGATGCTGTCCGCCGGGATCAATATCGTAGGATTTTCCTGGATG
TCCTGCCCTGCGGCTACTGAGCTTGAAGTAATCGTCCTTGACTGGTTAAG
CAAACTG

Seq. ID No: 18

>AADC_17ATGGTGTTGAGGTTATACGGCCAGGAAGGTCTTCAAAGCTAC
ATAAGAAACCATATTGCTTTAGCGAAACAGTTCGAGGAGTTGGTCATTCA
GGATTCCAGATTCGAGATGGTCACCCCACGTAGGTTTAGTCTTGTGTGCT
TTCGTTTACTGCCCAGAAGCACAGACGAGAACCAGGCGGATAAGCTAAAT
AGGGCACTGCTAGATGCGGTGAATCTTACGGGTGCGATATTTATCTCTCA
CACAGTCTTGTCAGGGATGTACGTGTTACGTCTTGCGGTGGGGGCACCGT
TGACTGAAGAGAGGCATGTGATAGCCGCGTGGAAGGTCTTGCAAGAGAAG
GCAACGGCATTACTGGAGGGTAATGTAGCGCAAGAACCTAACGGTCACGC
GCAATTATCCAATGGTGTCGTCGAGCTTGATGGTGCGCTAGATAATGGTG
TCACCACTGAACAGCATGGACATGTGGACGATCTTCCTATTAAATCTAAA

Seq. ID No: 19

>AADC_18ATGAGCCTTTCCCGTCACATAGACGCGGAGCGTTTAATCGAG
CAAATCAAGGAACACCCACATAAGAAACATTCTTCTGCGGAGTCCCGTAG
GGGTTTGCACAGGTCTTGGAGACGTGATGAGGATGCTGCGGACTTACCAA
AGTACACCTTACCCAAACATGGAATTAATTCAAAAGCCGCTTACCAACTG
TTACACGATGAAACTGCTCTGGACGGAAATCCTTTGCTTAACCTGGCGAG
CTTTGTGCACACCTGGATGCCGGAAGATGCGGACAAGTTAATAATGGAGA
ATATAAACAAAAATATAGTGGACATGGATGAATACCCTGCCGCCTCCCTT
ATCCACAACAGATGTATATCAATGCTGGCGGATTTATGGAAAGCCCCTAA
AGAAGGTAAAGTAATCGGCACGGCAACGGCAGGATCATCTGAGGCTATTA
TGCTAGGCGGTCTGGCTTTGAAGAAGAGATGGCAGGAAGCGAGAAAGGCT
GCTGGTAAGGATTATTTCCATCCGAACATTGTGTTCGGGAGCAATGCTCA
GGTAGCCTTGGAAAAATTTGCGCGTTATTTCGATGTCGAGACGAGACTTG
TGCCGGTCAAAGAGGAGAACGGCTTTGTTATGAATCCTCATGATGCCATA
CCTTATATCGACGAGAATACGATAGGAGTGATTGTGATACTGGGAAGTAC
TTACACGGGGCACTTCGAGGACGTCAAATTGATGAGTGATCTGTTAGATG
ACTTAGAGAAAAGAACTGGGCTAGACGTAAAAATACACGTCGATGGCGCA
TCAGGCGCATTCATAGCACCTTTCGCTTACCCGCACCTGAAATGGAGCTT
TGACGTACCTAGGGTTGTATCAATTAACACAAGTGGCCACAAGTTCGGGT
TAGTTTATGCGGGCTTAGGGTGGGTACTGTGGCGTGACGAGTCATTTCTA
CATCGTGACTTGGTTTTTGAGTTGCATTACTTGGGCTCAACCGAGTATAG
TTTCACTCTAAATTTCTCCAAGCCAGCGGCTCCAGTCATAGCGCAAATGT
TTAACTTTCTAAACTTGGGTTTTGAGGGATACAAGAAGATTGCATACAAG
GATATGAGAAACGCAAGGATGCTTAGCAGAGCCCTGGAGTCTACGACGTA
TTTTAAGGTATTCAGTAATATTCATGTCCCTCGTAACTCAGACAGTGCTC
ACGTTTCCAGCAACAACAAAGACGACCCGGAGACTTACCATGCAGGCTTG
CCCGTTGTCGCATTTAGGCTGTCTGACGAATTTAATCAGAACTACCCGAA
CGTTCGTCAAGTGTGGATTCAGACCCTGCTGCGTACGAAAGGTTGGATTG
TGCCCAATTATAACGCACCCCTTGGGGCTGAGAATATAGAGATTTTGAGA
ATCGTTGTCAGGGAAACACTTAGTGAGGATCTTATTGAAAGACTGATCGT
TGACATTGTCGCCGTTACCGAAAGTTTGACTACCGAGGAAGGTAACGTAT
TCGCCGGTATCACGGCGTCTGGCGCGGTGGTTAAGCCCGATCTGGACGAA
GCCCGTCCAGACAGTAGTAATTTTAACGATTCAGGTGACGGCGAAGCGCA
GGGCCAGACGGGTTATTCACGTCAGTGC

Seq. ID No: 20

>AADC_19ATGGCTCTATCTAAACATGTTAATACGGATAAATTGATTCGT
GATAGCAGAGATAAAAAGTCACCAAAGGAAAAGGCCCATCACACGGCCAC
TGCTCATCAGGAGGCAACTTACTCCTACGGGGATCGTTATGTCACTAACC
CCGTGCCTAAATACAACATAGCGAGTAAGGGAATTAGTGCCGACGCCGCA
TATAGACTTATTCACGACGAACTAGCCCTAGACGGGTCTACTGTGCTTAA
CCTTGCCTCATTTGTTCACACTTGGATGCCGCCCCAAGGCGAACAGCTAG
TACATGAAAATATTGCGAAGAACCTTATTGATAGCGATGAGTATCCTGCC
ACGCAGATCATTCATACTAGGTGCGTCAGTATTTTGGCTGATTTATGGCA
TGCACCGTCCGCTAAGCAGGCGGTAGGCACTGCAACTACGGGCTCCTCAG
AGGCGATACAGTTAGGCGGTCTGGCGATGAAAAAGATGTGGCAAGCGCGT
ATGAAAGCAGCTGGTAAAAACATCCACGAACCGGGTCCCAATATAGTCAT
GGGAGCTAACGCACAAGTGGCCCTTGAAAAGTTCGCGAGATATTTTGATG
TCGAATGTAGATTAGTCCCGGTAAGTGTAGAATCAAAGTATCGTCTTGAT
CCCAAGAAAGCTATGGATTTTGTAGATGAAAATACGATCGGAATATTCAT
CATTCTGGGTTCAACCTACACTGGTCACTACGAGCCTGTGAAAGAGATGA
GTGATCTGTTGGATGAGTACGAGAAACGTACAGGAATCTACGTTCCCATA
CACGTTGACGGCGCGTCTGGAGGGTTCGTAGCGCCTTTTGTCCACCCCAA
GATGGTTTGGGACTTTAAATTGCCAAGAGTAGTAAGTATAAATACATCTG
GGCACAAATTTGGCCTTTCATACGTCGGCGTTGGGTGGGTGGTCTGGCGT
GATAAAGCTCATCTGCCCAAGGACCTAATCTTTGAGTTGCATTACCTAGG
CTCCGTCGAGTACAGCTTCAGTCTGAATTTCTCTCGCCCCGCTGCACCTA
TCTTGGCCCAATACTTCAATTTAGTCCATCTTGGGTTCGAAGGTTACAGG
TCAGTCGGTTTAGCAGATATGAAAAATGCCAGAGAGCTTAGCCGTGCTTT
AGAAAAGACTGGATATTACACTGTATTGTCCGATATCCATAGAGCAGTAG
GCGCAAAGGACCCGCACGGGATTGACGACGCTGATATAGAGGCCTACGAG
CCCGGGCTGCCCGTTGTGGCCTTCCGTTTTAGCGATAACTTTAAAGAGAA
ACACCCTGAAATCCAACAGAAATGGATACAGACCCTGTTGAGGGCGAAAG
GGTGGATAGTACCAAACTACGAGTTACCACCTAGCCTTGAACAGATCGAG
ATACTTAGAGTCGTGGTTAGAGAAAACGTAACTGAAGTGCTTATCGACAA
GCTGATAGACGATATCGTCGAGATAACCGAACAACTTGCTGATTCAAGTT
CCTCAATGCACTCTCTGAATAATTTGGGACATATACAGAGGCCCAAGAAA
CACGAACACCCAGAAAGTAATTTAAAGGAAGGGGAGGGGTCCGATTACTC
AGGAACATACGCTCGTCCGTGT

Seq. ID No: 21

>AADC_20ATGGCCTTAAACGCAGTAAGCGCAGCTAGAGGTAGCGCAAGA
CAGTATATTTCCACGTTCTTAACGCTAGACAACGCTAAGAGCGGATTATT
TTATTATGTATTATTGGTTCAAGCCATTAAAGTGAAACGTCACCTGCGTG
CCAGAGGTATCAGTGCCTCCCTAAAAGAATTATACACATGGATTTCTCAG
CAGATCATACGTTTGCTTCTGAGGCTGCCGGCCACACGTAAGAAAGTTGC
TAGTCAGATGGATCAAGCAAAGTTGGATATCGAGAACAGACTGGTCCCCA
AAGGGGCCAATGTTACCAGGCATTTAAGTCTGCCGTCTGAAGGCAAGTCC
TTGGAATGGATTACACAGGAGATGGACAAGATGGATACCGAGCTTGGTGG
CACAAGTGACGCGTGGAGACAGGGGAAGCTATCCGGGGCTGTATATCATG
GAGGTGACGAGCTTGCGAAAATTATCGTCGCGGCCTATAGTAGATATTGC
GTCTCTAACCCACTTCATCCGGATGTTTTTCCAGCTGTACGTAAAATGGA
AGCTGAGATCGTTGCTATGTGTCTAAAGATGTATAGAGGGCCTGAAGGTG
CTGCCGGCGCTATGACGAGCGGGGGAACCGAATCAATCGTTATGTCCGTT
AAAACCCACAGAGACTGGGCAAGGAGTGTAAAGGGAATTAAAGAACCCGA
GATGGTTGTACCCGTTTCAGCCCATGCCGCCTTTGATAAGGCGGCGGCGT
ATCTTGGGATCAAGCTACATTCTATACCAGTTGATTCCTATACAAGACAG
GTTAACATAAAGCACGTGAAAAGAGCTATCAACTCAAATACTATCATGAT
AGTTGGCTCCTGCATCGGGTTCCCTGACGGTAATCAAGACGATATTGAAG
CCCTTGGTGCATTGGCCAAGAAGTACAACATAGGCCTGCATGTTGATTGC
TGCTTAGGCAGTTTCATTGTACCGTTTCTTGAGCCAGCGGGCCTAGCAAA
AGGCGATAACAAAGGAAGATACAAGTTGACTCCTTTCGACTTCACGGTAG
ATGGCGTCACCGCAATCTCCTGCGATACCCATAAGTATGGGTTTGCTCCA
AAGGGCACGAGTGTAATAATGTACCGTAGTGCGGAACTGCGTAGATTCCA
ATACTACGTTAACCCGATCTGGCCAGGGGGCGTCTACGCATCTCCATCTC
TTAGTGGGTCTCGTCCCGGTGCACTGATAGCAGGCTGTTGGGCGGTGATG
CAATATATGGGTACAGAGGGGTACCTTAGCAGTTGCCGTGATATAGTAAT
TGCTACTAGAAAGATAGCTGACGCGATTACAGATGATATCCCTGAGTTAT
ACGTCCTGGGTAATCCTCCGGCTTCAGTCGTAGCATTCGGGTCACGTAAT
CCAACTGTAGACCCTCTTGAAGTCGGAGATGGCATGCGTAAGAGGGGGTG
GCATCTGAACGGCTTGAGTTCACCAAAATCTGTCCATATTGCTTGTACCC
GTCTGACTTTACCCGTCGTGGACCAGTTCATTGCAGACCTAAAGGATTGC
GTCAGGGAAGCAAAGGTGGCACCTAGTGGGAAAGGTACGATGGTTTCCGT
GTACGGGCTAGGCAACAGCTCTGCGGTGGGACCCGACATGGTTTCCCAAC
TTGCCAGTGCCTTTCTGGACGCCCTGTACAAAGCA

Seq. ID No: 22

>AADC_21ATGGAACTAAAGACTGCGGCCAATGAGATCTGTAACAGTAAT
CAGATGTGTCAAGGTGAGAACTATTCACAGAACATGCTATTGAGAGATGG
GCTAATTGATTTGAAGAACCAGATCAAAGAAGGAGATGAGGGTCTAGGTC
ATGGCTTTAGCGACTTCTCAAACGTATTTGCATCAGACCTACTACCAGCG
CGTAACGGCGAAAAATACACAGAGGGATTCCTATTAGAGGTATTCAATAT
ACTATTCAGTTATATCAGGAAGACCTTTGACAGAAAATCCAAGGTATTGG
ATTTCCACCACCCCCACCAGCTGTTGGAGGGACTGGAAGGGTTTAACTTG
GAATTGAGCGATCAGCCAGAACCACTGGAGCAAATTCTTGGTGACTGTAG
AGATACGTTAAAATACGGGGTTAAGACGGCACATCCGAGGTACTTTAATC
AATTAAGCTCCGGCCTTGACATGGTGGGCCTTGCAGGGGAATGGCTTACG
GGTGCAGCAAATACCAATATGTTCACCTACGAGATCGCTCCCGTCTTCAT
CATAATGGAGGGACTACTTATAAAGAAGATGCATGAGTTGGTAGGGTGGG
GAGAGTTGGAGGCGGATGGGATATTCTCTCCAGGGGGCACCATTTCAAAT
CTGTACAGTGTTTTAGTAGCCAGATACAAATTTTTCCCTATAGTCAAGCT
AAAAGGTATGGCTGCGTTACCCAGAATCGTCCTGTTCACTTCTGAGCACT
CACATTACTCTTTTCAGAAGGCAAGTGCAACTCTTGGAATTGGAATAGAG
AATGTTATCGCTGTCAAATGCGATGAGAGGGGTAAGATGATACCATCCGA
TCTGGATGAGAAGATTATGGCACAGAAAGAGAAA

Seq. ID No: 23

>AADC_22ATGTGGAAAGCTAAGGGAACAAGAGGTTTCGAGCTACAGATA
GACAGTTGTTTGGAGAACGCTGAGTACCTGTACAAGAAACTAAAAAGCAG
GAACGGCTTTGAGCTTGTTTTCCCCGACGAGCCGGAGCACACCAATGTCT
GCTTCTGGTATATTCCCCCTAGTTTGAAGGGTATGCCCAGGGACAAGGAA
TGGAATACGAAACTTCATAAGGTAGCGGCCAAAATTAAGGCTCAAATGAT
GGAAGAGGGAACGGTCATGGTGTCATACCAGCCGCTTAAAAATAAGCCAA
ACTTCTTTAGGATGGTGTTCTCAAACCCAGCTTCCAAGAAAAGTGACATA
GACTTTTTGCTAGATGAGATAGAGAGGTTAGGAAGTGACCTTGAATTT

Seq. ID No: 24

>AADC_23ATGTTTGGATCTCAACATCAGATGGATGTTGCGGCTTTAGAT
AGACAGCTGAAAGAGGACAAAGAGTCCGGGAAATTGCCACTACTTCTGGT
TGCAAATGCGGGAACACCGGGTGCAGGACACACGGACAAGTTGGCCAGGT
TGAAAGAACTTTGTCATCAGTACAACATCTGGCTTCATGTTGAGGGAGTC
AATTTAGCGACCTTGGCCCTTGGGTATGTAAGCGCATCTGTGCTGGCGGC
AACGAAATGCGATAGCATGACCTTGACACTTGGACCTTGGTTAGGTCTAC
CAGCCGTACCGGCGGTTACCCTATACAGACACGAAGATCCGAGTCTTAGC
CTGGCTGCAGGGTTGACAACATCTCAACCCGTGGAAAAGTTGAGAGCCTT
GCCGCTATGGCTGAGTCTGCAGTACCTAGGCCATAACGGGATAGTTGAGA
GAATTAAACACGCCTCTCAATTATCCCAAAGGCTATTGGAAAATCTTAAA
GATGTGACATCCATTAAAACATCCGTGGAGCCCGATGGCAATTCTCCGGT
GGTCGTGTTTAAGTTTTTCTATGACGGACCAGGCTCAGGGTCCACCATTA
ATCTTAACACGATTGAACGTGAGTCCGACGCAATGAATCAGTGGTTGGGC
GAACAGTTGGCCGCCCTTATCCCAAGTTGCGCTGTAGATACTGTTGAGTT
GGAAGACGAAGGTGTGTGTGTGAGATTTAACCCTATGATGACATCTGCAG
TACTTGGCACAACCATAGAGGACGTAGATCAGCTGGTTGAATGCGTCAAG
GTTAAAATTCCCATAATACACAATACACTACAGCTAAAGGAGGAGTTCAG
GCTTGAAGTAGAAAGGATTGCGGGCCTAACATATGTTGTTGATTATTCTT
GGGCGGGGTTGGGCGTTTTACGTTATGATCACGTTTCAGAAGAGCTAGAT
GGGTCCAGACGTGAAGCCGAATTAGAGAAGATTAATGCTTCCCTATTAAA
GAAGTTGAATGAGCTTGAAAGCGACTTGTCATTTAGTTCCGGACCCGAGT
TTGGTGCCGAGAAAAATTGTGTCTACATAGGTATGGCAACAGAGGACGTA
GATGTTAGTGAATTGGTTGAAACAATTGCTGTCATGGGCAGGGAGATAGA
GGAAAATAGCAAACTGTTAGAGAACATGACTGAAGTAGTTCGTAAGGGCA
TCCTAGAAGCTGAGGTGCAACTACAGAAAGCAAACGAAGAAAGACTTCTG
GAGGAAGGCGTACTAAGACAGATCCCTTTGGTTGGCAGCGTGTTAAACTG
GCTTTCTCCTGTCCAGGCTACTCCTAAGGGCCGTACGTTCAATCTGACCG
CCGGTAGTTTGGAGTCCACAGAAATTACTTACGCTAGTAAGGCACAAGCT
AATGGAACTTCACCCCCACCAACACCAAGCCTAGGGCATGCCAAGAGACA
TCCTGGCCAGAAGCTGTTTAAGCGTTTGAGTAGAAACAGTGACGCAATGT
CCGAGACTAGCTCAGTTTCTCACCTAGAGGAAGTGGAAAATCTGGAGGCG
AGTCCCACCCCCGAGCCACAACCTGGACATCCTACCGAACCTCCTGTTCC
ATCCGTTGAATCAAACTCTGAGGAGCCACATGAGGCGGAAGCGTTAGATA
CTAAAACTGTAGAATCTGAAAGTCTACGT

Seq. ID No: 25

>ADK1ATGAGTTCTAGTGAGTCTATCCGTATGGTTCTGATCGGGCCTCCC
GGTGCTGGTAAGGGAACGCAAGCACCCAATTTGCAGGAAAGATTTCATGC
TGCTCATTTGGCTACGGGTGACATGCTTAGGAGTCAGATAGCCAAGGGCA
CTCAGTTGGGTTTAGAGGCTAAGAAAATAATGGACCAAGGCGGCTTAGTG
AGTGACGATATTATGGTCAATATGATTAAGGATGAACTGACTAACAATCC
AGCATGTAAAAATGGCTTCATTCTGGATGGATTCCCAAGAACTATCCCTC
AGGCCGAGAAATTAGACCAAATGCTTAAAGAGCAGGGGACACCCCTAGAA
AAAGCTATTGAACTAAAAGTTGATGATGAATTGTTGGTTGCGAGGATAAC
TGGTAGGCTTATCCACCCTGCGTCTGGCAGAAGTTACCACAAGATCTTCA
ACCCGCCGAAGGAAGATATGAAGGATGACGTAACAGGGGAGGCCCTAGTT
CAGAGATCTGACGATAACGCGGATGCTCTGAAAAAGCGTCTAGCCGCATA
TCATGCGCAGACTGAGCCGATCGTGGACTTCTACAAGAAAACGGGCATTT
GGGCGGGCGTAGACGCCTCTCAACCACCAGCCACAGTATGGGCAGACATC
CTAAATAAGCTGGGTAAGGAT

Seq. ID No: 26

>AOQS_1ATGTCTAATGCAGCCATAAGATCCTCCCGTGCCGTATCCGTAT
CATCCAGCACGAAGTATTACGACTTCACGGTAATAGGTAGTGGTGTTGCG
GGGTTAAGGTATGCCCTTGAGGTAGCCAAACAGGGTACTGTGGCAGTGAT
AACAAAAGACGAGCCACACGAAAGCAATACAAACTATGCACAGGGAGGAG
TAAGTGCAGTGCTTTGCCCGTTGGATTCAGTTGAGAGTCACATGAGAGAC
ACGATGGTGGCAGGTGCGCACTTATGTGACGAAGAAACCGTCAGGGTCGT
GTGTACTGAGGGGCCCGAGCGTATCCGTGAGCTAATAGCTATGGGAGCAT
CATTCGATCACGGTGAGGACGGGAACCTACACCTAGCGAGGGAGGGCGGC
CACTCCCACTGTCGTATTGTCCATGCCGCTGACATGACGGGTAGGGAAAT
CGAACGTGCTCTGCTTGAGGCAGTTTTGAATGACCCAAACATTTCTGTGT
TCAAACACCATTTTGCAATAGACTTACTAACATCTCAGGACGGACTAAAT
ACTGTCTGCCACGGGGTTGACACCTTAAACATCAAGACTAACGAAGTAGT
TAGATTTATCTCCAAGGTGACCTTACTAGCTTCTGGAGGTGCAGGTCATA
TCTACCCGTCAACAACGAACCCCCTGGTAGCGACCGGAGATGGTATGGCA
ATGGCCCATAGGGCGCAAGCCGTCATTTCAAATATGGAATTTGTCCAGTT
TCATCCCACTGCACTGGCTGATGAAGGCTTGCCGATCAAATTGCAAACCG
CGAGGGAGAACGCGTTCCTAATAACTGAAGCCGTGCGTGGCGACGGCGGG
ATTCTGTATAATCTGGGCATGGAAAGGTTTATGCCCGTCTATGATGAGAG
AGCTGAGCTTGCTCCTAGGGATGTGGTCGCGAGGTCAATAGACGACCAAT
TGAAGAAACGTAATGAAAAGTATGTACTACTAGATATCAGTCACAAACCG
AGGGAAAAGATCCTGGCGCATTTTCCCAACATCGCGAGCGAGTGTCTTAA
ACATGGATTGGATATTACTCGTCAGCCTATCCCAGTAGTGCCGGCGGCTC
ATTATATGTGCGGCGGTGTGCGTGCAGGCCTACAGGGTGAGACCAATGTG
CTTGGTCTTTTTGTAGCAGGAGAAGTCGCATGTACAGGCCTTCACGGTGC
GAATAGGCTGGCTTCCAATTCATTACTTGAAGCCCTTGTCTTCGCTCGTC
GTGCAGTACAACCCAGCACGGAATTAATGAAGCGTACACGTTTGGACGTG
TGTGCATCTGAAAAGTGGACTAGGCCAGTGGTGGCCACCGCGCGTCTGTT
AGGGGATGAAGTGATTGCCAAGATCATCGCTTTAACAAAGGAGGTCAGAC
GTGAATTACAAGAGGTTATGTGGAAGTACGTGGGGATAGTAAGGTCTACT
ATTAGACTGACAACGGCTGAGAGGAAAATTGCGGAGTTGGAAGCTAAGTG
GGAGACGTTTCTGTTCGAGCACGGGTGGGAACAGACGGTTGTTGCCTTGG
AAGCCTGCGAGATGCGTAACCTATTCTGTTGCGCAAAACTTGTAGTGTCA
TCTGCGCTTGCCAGACACGAAAGCAGGGGGCTGCATTATATGACGGATTT
CCCGTTTGTCGAAGAGAGCAAGCGTATTCCAACCATAATTTTACCGAGCA
GTCCAACTACTGCCAGCTGGTCAAGTAGGAGATTGCAAAATATTTCTTCA
AGCAGCTTGATAGATTGTGGGAGTGGCGAAGGACGTGGCTCTCTACTGAC
TTGCGGTGACGTTGAAGAGAATCCAGGACCTAGCAGCTCCTCTTCTAGTC
AAACAACAGAGTTAGTCCCCTATAAACTGCAGCGTCTTGTTAAAGAATTT
AAATCATTGACAGAACCGATTGACAGGCTGAAATGGGTGTTACATTATGC
GTCCCTTTTGCCACAGATGCCTGAGTCCTCAAAAACCGAATCAAATAGGG
TGATGGGCTGTACAGCGAGGGTTTGGCTGGATGCCGAATTAGGCCAGGAT
GGCAAGATGAGATTTTGTGCCGACAGTGATTCAGACGTGTCTAAGGGGAT
GTGTAGCTGTTTAATACAGGTTCTGGACGAGGCAAGCCCTGTCGAGGTAA
TGGAGCTTAAAACTGAGGACTTGGCAGAGTTAAATGTCGGTCTGCTTGGC
GGAGAAAGGTCACGTGTTAACACTTGGTATAACGTACTTGTCAGCATGCA
AAAGAAAACTAGAAGGTTAGTTGCAGAGCGTGAAGGGAAGGTGCCGTCCT
TTGAGCCATTTCCTTCGCTAGTACTAACCGCCCACGGAATCGAGGCAAAG
GGGTCCTTCGCCCAGGCGCAGGCAAAGTATTTATTCCCTGAGGAAAGTAG
AGTCGAAGAGCTAGTTAATGTATTAAAGGAGAAGAAGATCGGTGTCGTGG
CACACTTTTATATGGACCCAGAAGTGCAGGGTGTTTTGACAGCTGCACAG
AAACATTGGCCCCATATCTCAATCTCAGACTCTTTAGTCATGGCTGATTC
CGCGGTGACTATGGCAAAGGCTGGATGCCAGTTTATAACTGTCCTTGGAG
TCGATTTCATGTCAGAAAACGTGAGGGCCATCTTGGACCAAGCCGGTTTT
GAGAAAGTTGGAGTCTATAGGATGTCTGACGAAACAATTGGTTGCTCATT
GGCTGATGCAGCTTCAGCCCCAGCTTACCTTAATTATCTGGAGGCGGCGT
CCAGGAGCCCACCGTCTCTACATGTTGTTTACATAAACACTTCCTTGGAG
ACGAAAGCATTCGCGCATGAGCTGGTTCCTACAATAACGTGCACATCCTC
AAACGTAGTACAGACTATTCTGCAGGCTTTTGCTCAGATGCCGGAACTGA
CAGTATGGTACGGACCCGATTCATACATGGGCGCGAACATTGTCAAATTG
TTTCAACAGATGACACTGATGACAAATGAGGAAATAGCAAACATACATCC
CAAGCATTCACTGGACTCAATAAAAAGTTTATTACCGAGGCTGCACTACT
TCCAAGAAGGTACATGTATAGTGCATCATCTTTTCGGGCATGAGGTTGTA
GAAAGGATAAAGTATATGTATTGCGATGCCTTCCTGACAGCCCACCTAGA
GGTGCCCGGTGAAATGTTCTCCCTAGCGATGGAGGCAAAGAAAAGGGAAA
TGGGCGTCGTCGGTTCCACGCAAAACATACTAGACTTTATCAAGCAGAAA
GTCCAGGAAGCGGTAGACCGTAACGTCGATGACCATTTGCAGTTTGTTCT
AGGTACTGAATCAGGAATGGTGACGTCCATCGTTGCAGTAATTAGAAGCC
TATTAGGGAGCAGCGCCAACTCCAAACTTAAAGTGGAGGTAGTGTTTCCA
GTATCCTCCGATTCTATGACAAAGACTTCTTCTGACTCCAGCAATAGTAT
AAAAGTCGGAGATGTGGCCCTACCGGTGGTACCAGGGGTGGCGGGTGGCG
AAGGGTGCTCCATACACGGAGGCTGTGCAAGTTGCCCCTACATGAAGATG
AACTCCCTATCTAGCCTTCTGAAAGTCTGTCATAAACTGCCTGATTTGGA
AAATGTGTACGGTGGGTTTATCGCGGAGAGGTTCAAGAGACAGACGCCAC
AGGGAAAACTAATAGCAGATGTGGGCTGTGAGCCAATTCTACATATGAGG
CACTTCCAGGCAAATAAGGAACTTCCTGATAAACTGGTTCATCAGGTCTT
ATCATGCGAGTCCAAGCGT

Seq. ID No: 27

>AOQS_2ATGAATACCCTACCTGAACACTCATGTGACGTACTAATTATCG
GCAGTGGAGCGGCGGGACTGAGCCTGGCACTTAGGTTGGCGGACCAGCAT
CAGGTCATTGTACTGTCTAAGGGACCGGTAACAGAAGGTTCAACTTTCTA
TGCGCAAGGTGGCATTGCCGCGGTGTTCGATGAGACGGATTCTATAGACT
CCCATGTTGAAGATACCCTAATAGCCGGTGCCGGCATCTGCGATAGGCAC
GCCGTTGAGTTTGTGGCCAGCAATGCCAGAAGCTGTGTACAGTGGCTGAT
TGACCAAGGGGTGCTGTTTGATACCCACATTCAGCCTAATGGCGAAGAAT
CTTACCACCTGACACGTGAAGGTGGCCACTCCCACAGGCGTATATTGCAC
GCCGCTGACGCCACGGGCCGTGAAGTGGAGACAACTCTGGTCAGTAAGGC
GCTAAATCACCCGAACATCCGTGTGTTGGAACGTAGCAATGCGGTGGACC
TTATAGTCTCTGACAAAATCGGGCTTCCCGGGACTAGACGTGTCGTGGGC
GCCTGGGTCTGGAACCGTAATAAAGAGACGGTAGAAACATGCCACGCCAA
AGCTGTGGTTCTGGCTACAGGTGGCGCATCAAAAGTATACCAATACACGA
CTAACCCCGATATTTCTAGCGGTGACGGCATAGCAATGGCTTGGCGTGCA
GGCTGCAGGGTAGCGAATTTAGAATTTAATCAATTTCATCCAACAGCGTT
ATATCATCCTCAAGCAAGAAATTTTCTGCTAACGGAGGCGTTGAGAGGTG
AAGGTGCCTATTTAAAAAGACCTGATGGAACGAGATTCATGCCCGACTTC
GATGAGCGTGGCGAATTAGCCCCAAGGGACATCGTCGCACGTGCTATTGA
TCATGAGATGAAAAGATTGGGTGCTGACTGTATGTTCTTAGATATTAGCC
ATAAGCCAGCTGATTTTATCAGACAACACTTTCCAATGATTTATGAGAAG
CTATTAGGCTTAGGTATTGACCTGACCCAAGAACCTGTCCCAATAGTGCC
CGCTGCTCATTATACCTGCGGTGGCGTCATGGTGGACGATCACGGCCGTA
CGGACGTTGAAGGCTTGTATGCAATTGGGGAGGTGAGTTACACCGGGTTA
CATGGTGCTAATCGTATGGCATCCAACAGCCTGTTGGAATGTCTAGTATA
CGGCTGGTCAGCAGCTGAAGACATCACGCGTAGAATGCCGTACGCGCACG
ATATCAGTACATTACCGCCGTGGGACGAGTCTAGGGTCGAAAACCCTGAT
GAAAGAGTTGTAATTCAACATAATTGGCATGAACTAAGGCTTTTTATGTG
GGACTATGTGGGTATAGTAAGGACCACAAAACGTTTGGAACGTGCTCTTC
GTCGTATAACCATGCTACAGCAGGAAATAGACGAGTATTACGCTCACTTT
AGAGTCTCAAATAATCTTCTTGAGTTGAGGAATTTGGTCCAAGTCGCGGA
GCTAATCGTGAGGTGCGCAATGATGAGAAAGGAGTCCAGAGGGCTGCATT
TCACTTTAGACTATCCCGAACTTCTGACACACTCAGGCCCCTCTATATTG
TCCCCAGGAAACCACTATATTAATAGAGGTAGTGGTGAAGGCAGGGGATC
TCTTTTGACTTGCGGAGACGTAGAAGAGAATCCTGGACCCGGAAGTGTGA
TGTTCGACCCCGATACCGCGATTTATCCGTTTCCTCCCAAGCCCACACCA
CTATCTATTGACGAAAAAGCATACTACAGGGAGAAGATTAAGCGTTTGTT
AAAGGAACGTAACGCGGTGATGGTGGCCCATTATTATACCGACCCGGAAA
TACAACAACTTGCAGAAGAGACAGGGGGCTGTATCAGTGATTCACTAGAA
ATGGCAAGGTTTGGAGCCAAACACCCGGCCAGTACTTTACTGGTGGCTGG
AGTACGTTTCATGGGGGAAACCGCAAAGATACTATCCCCAGAAAAGACCA
TCTTAATGCCAACCTTGCAAGCGGAGTGTAGCCTGGATCTTGGCTGCCCC
GTTGAGGAGTTCAACGCATTTTGTGATGCCCATCCCGATCGTACAGTCGT
AGTCTACGCCAACACCAGTGCAGCCGTCAAAGCTAGGGCTGATTGGGTGG
TAACGTCAAGTATCGCGGTGGAGTTAATCGATCATTTAGACAGTCTAGGA
GAGAAAATTATTTGGGCCCCAGATAAACACCTGGGAAGGTACGTCCAAAA
GCAGACGGGTGGGGACATACTATGCTGGCAGGGTGCTTGTATCGTGCACG
ATGAGTTCAAGACGCAAGCATTGACAAGGCTACAAGAAGAGTACCCAGAC
GCAGCGATCTTAGTACACCCGGAATCACCGCAGGCGATTGTGGACATGGC
GGATGCTGTCGGCAGCACCTCCCAATTAATTGCGGCGGCAAAGACTTTAC
CTCACCAGAGATTAATAGTGGCTACAGACAGAGGCATATTTTACAAGATG
CAGCAGGCCGTACCAGACAAAGAATTATTAGAGGCGCCGACGGCTGGAGA
GGGCGCGACCTGTAGGAGTTGTGCACATTGTCCTTGGATGGCTATGAATG
GCCTTCAAGCAATCGCAGAAGCCCTGGAACAAGAAGGCTCCAACCATGAA
GTCCACGTGGATGAGAGACTGAGAGAACGTGCCCTGGTCCCTTTAAATAG
GATGTTAGACTTTGCTGCAACCTTGAGAGGA

Seq. ID No: 28

>ATMT_1ATGACACCGGCTGCCGGTAAGACCTTCAACACTTCCATCGCTG
GCGCCGACGACCTGATTAGACTTCATCTATCAGAGTCCCCTCATGGCGCC
AGTAAGGCAGCTCTTCAAGCCGCCGAACGTGAACTAGCCCGTGTCAATGT
GTATCCAGATCCGGAACGTCAGGAATTGGTCCGTGCTCTAGCCGCCCATT
GGGGAGTCGGACCGGAGCACATTGCGGTAGCAAATGGATCAGACGAGTTG
GTACTGGCGACAGCCCTAACTCTAGGTGATCGTAACCTTCCGGGTTTGGT
CACGGATGGGACGTTCCCCGGCTATCGTGCGTGTCTTGAACTGCTAGGGC
GTGGGTGCACCGCGGTCCCGCCTGACGGGACAGCCGTCGATGTGGCAGGA
TTCGCAGCCAGATTGCCCGGACATGGTATCGGCTACCTGTGTAACCCTCA
TAACCCATCAGGAGCAGCACTAACAAGACAAGAACTGGCGGCGCTGGTTG
AAGTGTCCGGGCGTAGTGGTGTCCCTCTTGTATTTGACGAGGCCTACATG
GAATTTGCGGGGCCGGATGTGCCACAGACTAGGGATTTAACAGCGGCCGG
AGACGCACCGGTAGTCGCGCTGAGAACGTTCAGCAAAGCCTATGGCTTGG
CGGCACTAAGAGTTGGATATGCCGTGGGTAGGCCTGACTTGATAGCCGGT
TTAAGAGGAACGCTTAGAGCACTGCCGTTCAGTGTTAACCGTTTGGCTCA
AGCAGCGGCCATAGCAGCACTGGGAGACCCTGATTTTGTTGACGGAGTTA
GGAGAAGCACTGCTGAAAGGCGTAGGTGGTTTGTAGGCGAGTTGGACAGG
CGTGGCAGGGCTCATTTGCCGTCCGTAACGAACTTTGTCGCCGTTGCAGC
TAGAGACTGTGCGCGTGCACAGGACAGACTGGCTGCTGACTTCGGTATCT
TGGTCAGAAACGCGGGCTTATTCGGGTTCCCTGGCTACCTGAGGACTTCA
CTAGGCGAGAAGAAGGACTTAGAGAGGTTTTTGGACGCTCTGGATGAGAT
TGAACAAAACCCCGGTGGCGGATCAGGTGAAGGGCGTGGCTCTCTACTTA
CGTGTGGTGACGTAGAAGAGAACCCGGGTCCCATGACTGCCCCGTTAAGC
CGTGATGGTTTACGTGCAATGGGTGAGTCCGTCTTTCGTCCAGCGGAGTG
GCAAGGCGCGGCGCATACGCCGTTGGACGCGGATACAGCTTTCAACGGCT
TTATCAGTACACATGTTGTTTTTGCCCTTGAACAACTAGGTTTGTTCGCC
TGGTTTGATGAAAGCGATAGGTTAGATGTTCCTCAGTACTGCTGGCGTCG
TAAGTTAGATGAACGTGTTTTTCGTCAACTTGTGAGTGCCGCCGAGGCAT
TCGGTTATCTTGATGTTCATGATGACTTGGTGACCCCAACGCCAGCTTGG
TCCGAACTGAGGAGAAAAATTGGCTTCTTCACCTGGGGCGTAGGCGGATA
CCACGATGTATTTGCCAACGCGGCTTCAATAGCTCGTGGGGAGAGGGCCT
TCGGTAAAGACGTGCTACGTGATGAGGCTATGGTGGCCCTAGGCTCAGCA
CAAGCGGACATGGCGCTGATGAGAGACCTGCTGGATGAGCAGATTGCTGC
GTTGGACTTTTCCGTAATAGCCGATCTTGGTTCAGGTATAAGCGAACGTG
TCTGTCGTCTGGTGAAGTCAAGACCGGGAGCCAGAGGTCTTGGGGTCGAT
ATATCAGCGTCTGCGACCGCATTGGCAGCTGGGACGGTTGAACGTCACGA
ACTAGCTGATAGAGTTCAACCTATTTGCGCAGACGTCTTAGATGTTCTGT
TTCACGGCAGAAGGATAGAGGGTGCGGACCAGGTCGATGTAGCAATGTCA
TTCATGTTCCTGCACGACTTGCTAGTGGACCCTACGACCAGAACTGACGT
GATCCCAGCACTTAGGAAGGCATTTCCTAGGGCCCATACGTTCTTGCTGG
CTGATACGACAGTTCGTCCTAGGGACGAGAAGGATACATTGCCCGTATTC
TCAAGCGGTTTTGAGTTAGCACATGCGTTAATGGGCGTCCCAATTTATAC
GAGGGAGGAATATGAGAATCTTTTCCATGAGGGAGGTCTTCATCTGCGTA
GGACCGTTCCGTTTGGAGCGCCTCATACATATTTATTCGTTCTTGAAGCT
CAG

Seq. ID No: 29

>ATMT_2ATGCAGGCATTGCCCGTTAAGGGTGACACTGTAAGCCGTCCTC
CCACTGTCCACAGCCTGCACCATGAACATGAAAGGGCTGATGGAATGTTG
AGACTGCACTGTAACGAAAATCCTTACGGTCCGCCTTCCGGTGTTATCGC
TTCTGTAACTAAAGAACTGGAGGGAAGATGTAGCACGTATCCTGACTCCG
AAGTCACTGCTCTGCGTGAGGCTTTAGCTGGGCAGGTAGGGGTTGGTACC
GACATGGTAGCTGTGGGTAACGGGGCTGATGAATTAGTCCTACTTATTAC
GTTAGCCTCTGCGGGTCCTGGGGATACTGTGGTTGTGACCGAATCTACGT
TTCCAGGTTATGCAGCTTCAGCTGCAGTGGCAGGCGCGACTGTAAGAGGG
GTGCCTCTACACCGTGACCGTGTATCTGCGACAGCGTTGGTAGAAGCTGT
CGATGATGGAGCGAGGTTAGTCTTTGTATGTAACCCTCATAACCCAACGG
GTACCGTGCTTAGCCCCGCAGCAGTCGAAGAAATCTTGAGGGCATGTGAG
AGGACTGGTGCGGTGCCTGTTTTTGACGAAGCATACATTGAATTTGCTGG
TCCCGGGTTTGATCATGCATTGGATGCAGTCAGAGCCGGGAGACGTCTAC
TGGTACTGAGAACTTTTTCAAAGGCCTGGGGACTGGCAGCACTGAGGGCG
GGGTACGCCGTCGGTCCAGCCGATCTTGTCGCGGGTATAATGGAGGCCAG
GAGACCCCTGCCATTTTCCGTAAATAGGTTGGCGCAACAGGCTGCACTTG
CCGCGCTAGGATCTCCCGATCATATTGCAGAGGTCTACGAAAGGACAACA
CGTGAGAGGGAACGTCTGTGTCGTGCTCTAACTGGCCTGGGGGTCGCATA
TGTGCCGTCCGTTACAAACTTTGTAATGGTGAAGACTCCTGGAAACTCCA
CGAGATTTGCTTCTCGTTTGGCTGATGAGCATGGAATCTTAGTAAGGGAT
CTAGCACCCTTCGGTTATCCTGGTCACGTGAGAGTAAGCGTGGGTACAGC
TGAGGACACTGATCAATTCTGCGCAGCCCTAGGAAGCCTTCTTGCTTCTC
CGCGTTCTCATGCCGCCACCGGGCATGGTTTGGGGGCATCCTCTGGTGCT
GGCGGAGCGGGAAACGCTGCCATCAGGTCCGCGAGAGATGTTCTGCCGGT
ACCAACGCTTGATCCCGTCGCTCCTCAAGACCTTTTCAACGGTTATGTCG
GGGCTCATGCAGTATTCGCGCTAACTCGTTTGGGCGTATGGGATAGATTG
GCGGAAGGTTCTGAGCCAACTGTAGATGCCTTAGCGGTTCAGGCAGGAAC
GGATGCTACAGGCCTGATGCCACTACTGCGTGTTGCCGCCTTACTGGGCT
ACGTGAGCTTAACTGACGGTTCCGCCCCTGCTGTACGTTTGACTGAGTCT
GGGAGGGAACTTGTCCGTATGCGTGGCTTTTTCACGTGGGGCGTCGGAGG
ATATCACGAAGTTCTTAGATCGTTGCCGGCATTGGCCAGGGGGACCTCCG
TATTCGAGCAGGACGTCGATCGTGATGGAGGGATGGTAGCCGTGGGGTCT
GGCGAGGTCGGACGTGAAATGATGCTTCCTTTGGAACAAGAAGTCCTTGC
AACAGTTGACTTCAGGACTGTGGCCGACTTGGGATGTGGAGATGCCACTA
GGTTGTTGAGGTTGTGCGATGGACACCCACACAGGAGGGGGACAGGCATC
GAAATTAACCAAGGCGCTTGCGTACAGGCTAATAAGCGTGTTGCAGACGC
GGGGCTGGCTGATAGAGTGGACATTGTGCATGGTGATGCTCTTGACCTTT
CAGGACGTACGTTTCCGGAAGTCGATCTAGTCACTTCCTTCCTGATGATG
CACGATCTATTTGACGCGACAGGTGACCCCGTGGGAGTCATGAGGACCTT
GAGGGAGGTCTTCCCGAGAGCGCGTCACTTCCTTATAGGGGATACAGTAG
CCCAGGACTGGGAAGAGAGACGTGAGGGTTTGCCAATGTTCTCAGTGGGC
TTTGAATTGGTTCACGCGTTTATGGACACTCCTATCATGAATAGGGGCAC
CTACGAGGACGCTTTCGCGGGCGCCGGTCTTCGTGTAGCCAGAAGGGAGC
CACTAGGGGCTCCTAGTACGTGGCTTTGGCTATTGTCTACAGAA

Seq. ID No: 30

>ATMT_3ATGAGGCGTCGTTGGGCCGTTACGGCCTCTGCATCCTGGTGGG
GGACCGCCTGTGAACTTCATGCCAGTGCGTCTGCCGCATATACGCCACCG
TGCCACTCTCCGGGTACAGGAGGAAGGGGTACCGAGTCTGGCCCTATGAC
GGCACCTGTCCGTCAGGAGACGAGAAACTATAACGCATCTGTACCATCCG
CTGACGACCTTGTCCGTTTACATCTTAGCGAGAGTCCGTACGGTGCGTCT
CCCGCGGCAGTGGCTGCGGTCACAGGGGAGTTAGAGAGAATAAATAGGTA
TCCAGCTCCGGGCAGAGAGGGTTTGGTCCAGGCTTTGGCAAGACACTGGG
AACTACCCGAGGAGCATATAGCTGTCGCCAATGGGAGCGACGAGTTAGTT
CTAGCCACGGCGCTAACTTTAGGTGACCCCGGGTCACCTGGTCTAGTCAC
CGCGGGGACCTTCCCTGGGTACCTTGCAGCCCTAGAAAGAATAGGACGTG
GTGCGGTACAGGTTCCCTTAGCAGGATCTGGAACCGACACTGCAGCCTTT
GCAGACCGTCTTCCAGGTTGCGGAATTGGTTACGTGTGTAATCCACACAA
CCCTTGCGGGTCCGCATTGACCCATGATGAACTACACCGTTTAGTCGCCG
CGGCGCGTGACTCTGGGACCCCATTGGTGTTCGACGAGGCATACCATGAA
TTTGGGCCCCCGGCGCAGCCCCAAGCACGTACCCACCTAAGGGAGGATAC
TCCAGTCTTAGCTCTAAGGACGTTCAGCAAGGCATACGGCCTGGCCGCAC
TTAGGATAGGGTACGCGTTGGGTCCTGCCGATCTGATTGCTGAGGTGAGG
AGAACATTAACTGTGCTTCCTTTCAGCGTGAATAGAGCAGCGCAGGCTGC
CGCTCTTGCTGCATTAGATGATCAGGAGTTCTTAGGTAGCGTGAGAAGGG
ATTCAGCCGCCAGACGTCAGTGGTTTTGCGCTGAGCTGGAGCGTAGGGGT
TATAGATACCTTCCGTCTGTTACGAATTTTGTGGCGGTAGAAGTTGCTGC
CTCTGCAGAGGCACAAGACGTGCTGGCAAGGGATCATGGTATTTTGGTGA
GAGATACCGGAATGTTTGGATTCCCCGGTCACCTGAGAGTGAGCTTGGGC
TCAGTGGAAGAACTACGTGGATTCCTGGACGCCCTGGATAGAGTTACGGC
TGGCAGTAGGGGTGGGGGCTCTGGAGAGGGAAGAGGTTCCCTTCTAACTT
GCGGGGATGTAGAAGAAAACCCAGGACCGATGACCGGGCCAGTTAGTACT
TCAGCTCCAAGTCGTTGGCCAAGAACTTGGAGGCCTAACAGGCTGGAACC
CACCTCACGTGGTGGCCAACCAGGTCATGCAGCGAGGAGGAGTCCTGCAG
CGGGACGTAGGAGGAGAAGGGCTTCAGAAGCCCGTCCGCCCCCTAGCGGA
AGACAACCGGCTGTGAGGACGGAAAGGTGTGAGAGAGTGAGCCCGTTAAA
TACGCTGCCAAGTGAATGGCAGGGCCAGGCACCTACTCCACTGAACCCAG
ACACTGCATTCAATGGGTACATCTGTGCTAACGTATTGCATGGACTTGAG
CGTCTTGGAGTATTCGAACTTTTGAGGGATGAAAAGAGCTTAGATATGGA
TCGTTTCTGCGAGACGAATGGTCTCGATTCTGCGGTTTTTAGGGCGCTTG
TCGGAGCTGCAGAGTCATTCGGCTATCTTGACGTCAGAGGAGCCCAAGTA
AGAGCCACGTCTGTGGGTGAGGATGTTGCTAGATACTTGGGTTTCTTTAC
GTGGGGTGTTGGGGGATACCACGATATTTTTGCAAGTGCTGCTCCTGTCG
CCAGGGGCGAGAGGAGGTTTGGCGTTGATTTGCACAGGGATGAGGGGATG
GTCGCCCTGGGAAGTGCCCAGGCTGATACCGCGTTAATGAGACACATCCT
TGACGAGGAAATAGCAGGTATAGACTTCAGAACCCTGGTCGATCTTGGAG
CCGGGGTATCCGAGCGTGTGAGTAGACTCGTGAAGGCACGTCCTGGTACT
CGTGGGATCGGGATCGATATATCACGTCCAGCCACTGAACTTGCCAGAGA
CACCGTCGCAGGATACGGCCTGGCGGGAACGGTCGAGCCTGTGTGCGCCG
ACGTCTTGGACATATTGTTCAATGGTCAAGAGATTGACGGTGGGGATGCA
GCAGACGTTGTAATGTCATTTATGTTCCTACATGACTTATTAGCTGCGCC
CGAAAGAAGGGAAGAAGTAGTACCTAGATTACGTAAGGCTTTTCCGAGGG
CCCACACGTTCTTGTTGGCTGACACTACGATAAGGCCACGTAACGAGGAA
GGAGACGGCAGGTTGCCTGTCTTCTCATCCGGGTTTGAACTTGCTCATGC
CCTTATGGGTGTTCCTCTGCACACCAGAGAAGAATACGAAGAACTATTTG
AACGTGGAGGTATGAAGCTACGTAGAAGTGTACCTTTCGGGGCGCCTCAT
ACCTACCTATTCGTCCTGGAGGCGAGT

Seq. ID No: 31

>ATMT_4ATGACAAACGACCCCTCCCCAAGAGATGCACGTGACGAACTGC
CTGTGAGAGACGAGCTGCGTGGTCAATCCCCTTATGGGGCGCCGCAATTA
GACGTTCCGGTCAGACTAAATACTAACGAGAATCCGTATCCTTTACCGGA
GGCCCTGGTCGAAAGGATCGCGGAGAGAGTCAGGGAAGCCGCAAGGAGCT
TGAATAGGTATCCCGATAGGGATGCCGTCGAACTAAGGACTGAACTGGCA
CGTTATTTGACGAGAACAGCTGGGCATGAAGTTACCGCCGCGCATGTTTG
GGCAGCGAACGGATCAAATGAGGTCCTTCAACAGCTACTACAGACTTTCG
GGGGCCCAGGTCGTACTGCTATAGGCTTTGAACCATCATACTCAATGCAT
GCACTGATATCAAGATCCACTGGCACAGGATGGATCTCCGGGCCAAGAAA
TGACGATTTTACGATCGATGTGGATGCGGCTAGAGCAGCTATCGCCGAAC
ATAGACCAGAGGTCGTGTTTATTACCTCCCCCAACAACCCGACAGGTACT
GCAGTTAGAGCAGAAACAGTACTAGCGTTATATGAGGCAGCACAGGCAGC
AAGGCCGAGCATTGTAGTGGTGGATGAAGCATACGGGGAGTTCAGCCATC
ATCCCAGTCTACTGCCCCTTATAGAAGGCAGGCGTCATTTAGTTTTGTCC
AGAACAATGTCTAAGGCGTTTGGAGCTGCTGGTTTGAGGTTAGGATATCT
GGCTGCCGATCCAGCCGTGGTTGATGCTGTTCAATTAGTGCGTTTACCTT
ATCATTTATCCTCTGTTACGCAAGCTACCGCACTTGCAGCCTTGGAACAT
ACTGATACCTTGTTAGGCTATGTCGCCCAGCTTAAAGGTGAAAGAGACAG
GTTAGTTGCTGAGCTGAGAGCCATCGGGTACGAGGTAACGGAAAGCGACG
CGAACTTCGTACAATTTGGCCGTTTTGATGATTCACATGCTGTCTGGCGT
CAGATATTGGATAGAGGTGTTCTGGTCAGGGACAACGGCGTACCTGGGTG
GTTGAGGGTAACTGCTGGTACCCCAGAGGAGAACGATGCCTTCTTAGACG
CCGTTAGAGAGCTTAAAAAGGAGCACGATGCCGGCGGTGGAAGTGGGGAA
GGGCGTGGGTCCCTGCTTACTTGCGGTGACGTAGAAGAGAACCCCGGCCC
ATCTAGTTCATCTTCCACACGTACTGATTTTGCCCAGTCTGCAGTGGCCA
GCATCTTTACAGGTGCGATAGCGTCACACGCTGCGGTTTTAGCAGATGAT
CTGGGATTGTTTGATGCACTAGCTAAAGGCAAGCTGAGGAACAGAGACTT
AGACCGTTCCCCTTGGTTGAGAAATCGTATCAGGATTAGCGGTGCCCTAG
AGGCTCTGTGCAGGGTCGGAGCCGTCCAGAGATGCACAGACGGCTACGAG
CTGACCGACGTAGGGACGGAACTGGCTGGCCAGGTTCCAGTATTCCGTTT
GTGGCTGGGAGGTTACGCTTCAGTGCTGGCTGGGCAAATTTCAATCGGCG
CTGACCCTGCGACGGGTGTCCACGGGGGAATCGTGGCCGAATCAAGCGGT
GCTATCGGGGCACGTTACCTAGATGAGACGATAGTCAACTTGTTAGAGTC
ACTGAGGCCAGAGGGTAGGATATGCGATATTGGATGTGGAACCGGGGCTA
GACTGTTAAGAGTCTGTCGTAGAGTCAATCAACCCGGTATTGGATACGAT
TTGTCAGCCAAGGCTGTGGAAGCAGCAAGGGAGACTGTTGATGAAGCCAG
AAGGATTGGTGTAGATATCGACGTGAGGCAGGGAGACGCCACTGCGCTTA
CTCAAGATCATCCTGATGTGGACATAGTGACCCAGGCGTTTATGACTCAC
CACATAGCTCCTGACGAATACTGCGCGGCCGTTTTGAGGAGTTATCGTTC
CCGTTTTCCCAGAGCTAGGTATTTGGTTATTTTCGACACTGTTCCGTCTC
AGGACTCCGAGGAACCCGAAATCTTCGCCCCCGGCTTCGACTATATTCAT
GCACTACAAAATATGGAGCCGAGGTCACGAGGGGCGGCTAGGAGAATGTT
CACGGAAGCGGGTTATATTTGCCGTGAGGAAGTTGAGTTGGCTGTTCCCA
ACAGTTACGCGTGGGTTCTGGAGATGCGTGATAGAGAAGGCCCGGCTTCC

Seq. ID No: 32

>ATMT_5ATGACCAATGACCCTTCCCCACGTGATGCCCGTGACGAATTGC
CGGTGCGTGACGAATTGAGGGGACAAAGTCCATATGGTGCACCACAGCTA
GATGTTCCGGTTCGTCTTAATACTAACGAGAATCCTTACCCACTTCCTGA
GGCATTAGTAGAAAGGATCGCTGAGAGAGTAAGGGAAGCAGCAAGGTCCT
TGAACAGGTACCCAGATAGAGACGCCGTAGAATTAAGAACCGAACTAGCG
AGGTACCTGACAAGAACCGCAGGCCATGAGGTTACCGCTGCACACGTATG
GGCCGCAAATGGCTCCAATGAAGTACTTCAGCAGCTTCTGCAGACTTTCG
GAGGGCCTGGCAGAACTGCTATTGGGTTCGAACCGTCTTACTCAATGCAC
GCTCTGATTTCTCGTAGCACAGGGACCGGATGGATAAGCGGACCAAGAAA
CGACGACTTTACGATCGACGTTGATGCCGCCAGAGCTGCCATTGCCGAGC
ACAGGCCCGAAGTGGTTTTCATCACCTCCCCCAACAATCCAACCGGTACG
GCAGTGCGTGCCGAGACAGTTCTGGCGCTATATGAAGCGGCGCAAGCGGC
CCGTCCCTCCATTGTGGTAGTGGATGAAGCATACGGCGAGTTCAGCCATC
ATCCTTCACTGTTACCATTGATAGAAGGTAGGAGGCACCTTGTCCTATCC
CGTACTATGTCCAAGGCCTTTGGGGCAGCCGGTTTAAGATTGGGGTATCT
GGCCGCCGACCCAGCAGTGGTCGATGCGGTACAATTAGTCAGGCTTCCCT
ATCATTTAAGCAGTGTCACCCAAGCGACTGCCCTGGCAGCGTTGGAGCAC
ACGGATACGCTACTAGGATATGTAGCCCAGCTGAAGGGCGAGAGAGATCG
TTTGGTTGCCGAATTAAGAGCGATAGGTTATGAAGTCACCGAGAGTGACG
CAAACTTTGTGCAGTTTGGCCGTTTTGATGATAGCCATGCAGTTTGGAGG
CAGATCCTGGACAGGGGAGTGCTGGTGAGGGACAATGGTGTGCCAGGTTG
GTTAAGAGTAACAGCCGGCACACCGGAGGAAAACGACGCATTCTTAGACG
CGGTTAGAGAGCTTAAAAAGGAGCATGATGCGGGCGGGGGATCAGGAGAG
GGGAGAGGTAGCTTGCTGACTTGCGGTGACGTCGAGGAGAACCCGGGGCC
TGCTCAGGCGGCACCCACAACGGTGACAGAGGTGTTCAATCACGCGATAA
CTGCATCCGCTATCAGTGCAGCTTGGGAGATGGGGGCCTTTGACGCTCTG
AGGGTTAGTGAGAGGCTAGACGCTGATGAGTTTGCTGCGCGTGAAGGACT
TGACACTAGATCCACACATGAACTTTTTCGTGCTCTGGCTGCTGCTGACA
TCGTATCAAGAGACGGCGCGCAGATTCGTCGTGGTCCGAATTTCGCTGAA
GCTGATAGATGCAAAAGCCTGTTTCACTGGATGACAAGAGGGTGCGGGGA
ATTATTTAGCACACTGCCTGCGCTAGTTCGTGAGAAAAATAGAGTGGGTT
CCTTTTACCGTAGAGATGCCGCAGCCATCTCAGTTGCATGCCGTGAAATT
AACGCGGAATGGTGGGACCCTGTTTTCTGGCCAGTCGTCTCTGGTCTTGA
TTTCACGAGTGTTGCAGACCTTGGATGCGGTTCTGGTGAACGTTTGATTC
GTTTAGCTAGGACGGGACCGGAGGTGATGGCCCTAGGTATTGACTTTGCC
GCTGGGGCGATCGAAGTTGCTACGGCGGCTGTTGCTGAGGCTGGTTTGTC
AGACCGTATCTCATTAGTCCAGGGCGATGCCACAGCTTTAGAGCCTCGTC
CCGAATTTGCTGGTGTAGATTTACTAACTTGTTTCATGATGGGGCATGAT
TTCTGGCCGAGAGCAGAGGCAGTGGCTAGTCTACGTCGTATAAGGGAGGT
TTTTCCGGATTTGAAACACTTCCTTCTTGCAGATGCAACGAGGACGACGA
GTTATCCTGATACGGATATGCCTGTTTTTAGCATGGCATTCGAACTTGCG
CACGCAGTGATGGGAGACTACTTACCTACCTTAGAAGAGTGGAGACCCGT
GTTTGAAGAAGCCGGTTGGAGATGTGAGGGGGAGCATCCTATCAGCGTAC
CTGCTGATAGCGTAATGTTCCACTTGGTACCTAAT

Seq. ID No: 33

>ATMT_6ATGACAAATGATCCTTCACCGAGGGACGCGCGTGACGAGCTAC
CTGTACGTGACGAATTGAGAGGTCAATCACCCTACGGCGCTCCTCAGCTA
GATGTTCCAGTTCGTTTGAATACGAACGAAAACCCGTACCCACTACCCGA
AGCTCTGGTGGAGCGTATTGCTGAGAGAGTGCGTGAGGCCGCGAGAAGTT
TAAACAGGTACCCTGACAGGGACGCCGTTGAGCTTAGAACAGAATTGGCT
AGATACCTAACTCGTACCGCGGGTCATGAAGTTACAGCAGCACATGTCTG
GGCCGCCAACGGGTCTAATGAAGTTCTGCAGCAACTACTGCAGACGTTCG
GCGGACCGGGCAGGACAGCTATCGGCTTTGAACCTTCCTATAGCATGCAT
GCGCTGATATCAAGATCTACCGGGACGGGGTGGATCTCCGGACCCAGGAA
CGACGATTTCACGATAGATGTAGATGCAGCTCGTGCTGCCATCGCCGAGC
ATAGACCAGAAGTCGTATTTATTACCAGTCCTAATAACCCAACCGGGACG
GCGGTACGTGCTGAAACTGTACTTGCTCTGTATGAGGCGGCTCAAGCAGC
AAGGCCTTCCATTGTTGTTGTGGATGAAGCGTACGGCGAATTTTCACATC
ATCCGTCTCTGCTACCGCTGATAGAGGGTCGTCGTCACCTGGTATTGAGT
AGGACGATGTCAAAGGCCTTTGGGGCGGCTGGACTGAGACTGGGCTACCT
AGCCGCAGATCCTGCGGTCGTCGATGCAGTTCAATTGGTAAGGTTACCCT
ACCACCTATCTAGCGTCACACAGGCTACCGCTCTAGCAGCATTAGAACAT
ACTGATACACTGTTAGGCTATGTGGCTCAGCTAAAAGGGGAACGTGATAG
ATTAGTCGCAGAGCTTAGAGCGATCGGATATGAGGTGACCGAGAGTGATG
CGAACTTTGTGCAGTTTGGGAGATTTGACGACAGTCACGCTGTCTGGAGG
CAAATTTTGGACAGGGGAGTTTTAGTTAGAGATAACGGGGTACCCGGGTG
GCTGAGGGTAACTGCTGGGACACCAGAAGAAAATGATGCTTTTCTGGACG
CAGTCCGTGAGCTGAAGAAAGAGCACGACGCAGGAGGAGGATCTGGAGAG
GGTAGAGGCTCCCTATTGACATGCGGCGACGTCGAGGAAAATCCGGGCCC
ATCCACTGAGGTTAGCGAAGCCCAGGCAAGGAGGGCGGTAGCGGACATTT
TTAATAGCACTTTGGCGTCTTCCGCTATTGGCGCCGCTTGGGAGCTAGGG
GCACTGGACGAACTGAGAGAAAATGGGAAACTAGACGTGAGCGATTTTGC
GGTTCGTCACGATCTGCACGAACCTGCTGTAGTAGGGATGTTTACGGCGC
TGGCAAGCGTTGGCATTGTGCGTCGTGAAGGCGCCACCGTTGTTGTAGGT
CCTTATTTCGACGAGGCAAATCACCACCGTTCCTTATTTCATTGGCTTAA
CCAGGGCTCTGGAGAACTGTTCAGAAGGATGCCACAAGTGCTGCCCAATG
AGAATCGTACTGGAAAATTCTACCAACGTGACGCCGGAGCCATCAGTTAC
GCCTGTCGTGAGATCTCTGAACGTTATTTTGACCCAGCATTCTGGGCGGC
AGTAGATGGTCTTGGCTACACCCCAACTACAGTCGCAGACCTTGGCTCAG
GCTCAGGGGAGAGGCTGATTCAGATTGCGAGAAGATTCCCGGGGGTGAGG
GGTTTGGGGGTAGATATCGCCGATGGAGCCATCGCTATGGCCGAAAAAGA
GGTAGCCGCTAAAGGGTTCGGTGATCAGATCTCATTTGTCAGAGGCGATG
CGCGTACCATCGACCAAGTATCTGCCAGAGGTGAATTTGCCGAAGTGGAT
TTGTTAACATGCTTTATGATGGGGCATGACTTTTGGCCCCGTGAGAATTG
CGTACAGACCTTACGCAAATTGCGTGCAGCGTTCCCGAATGTGAGGAGGT
TTTTACTGGGAGACGCAACAAGGACGGTCGGGATACCGGATAGAGAGCTG
CCGGTTTTTACTCTGGGTTTTGAATTTGGTCATGACATGATGGGGGTCTA
CCTACCCACCTTGGACGAATGGGATGGAGTATTTGAGGAGGGAGGCTGGC
GTTGTGTAAAGAAGCATGCAATCGATAGCTTATCTGTGAGTGTTGTTTTC
GAACTAGAG

Seq. ID No: 34

>BH4reg_1ATGGCAGCGTCAGGGGAAGCCCGTAGGGTGCTAGTATATGG
TGGTAGGGGAGCCCTGGGTTCCCGTTGCGTACAGGCTTTTAGAGCGAGAA
ACTGGTGGGTGGCTAGTATCGATGTGGTCGAGAACGAAGAAGCTAGTGCT
TCAGTGATAGTTAAGATGACAGATTCATTTACTGAGCAGGCTGATCAGGT
TACTGCTGAAGTTGGGAAACTTTTAGGGGACCAGAAGGTAGACGCAATCC
TTTGCGTCGCAGGTGGGTGGGCTGGCGGGAATGCGAAAAGCAAATCTCTG
TTTAAAAATTGCGATTTAATGTGGAAGCAATCAATCTGGACCAGCACCAT
TTCTTCTCATCTAGCTACTAAACACCTAAAAGAAGGCGGGCTGTTAACAT
TGGCAGGAGCAAAAGCCGCCCTTGACGGTACCCCAGGAATGATTGGATAT
GGTATGGCTAAAGGAGCTGTTCACCAGCTATGTCAAAGCCTGGCTGGTAA
GAACTCTGGAATGCCGTCCGGCGCAGCGGCGATAGCAGTATTGCCCGTAA
CCCTGGATACTCCTATGAATAGGAAGTCCATGCCTGAAGCCGATTTCTCT
AGTTGGACTCCGCTGGAATTTCTAGTAGAAACTTTCCATGATTGGATAAC
CGGAAATAAGCGTCCTAATAGTGGTAGTCTAATACAAGTCGTCACCACGG
ATGGGAAAACCGAGCTGACCCCAGCGTACTTT

Seq. ID No: 35

>BH4reg_2ATGACCGCTCTTACCCAAGCTCATTGTGAAGCCTGCCGTGC
GGATGCACCTCACGTTAGCGATGAGGAGCTTCCAGTTTTGTTAAGGCAAA
TACCTGATTGGAATATAGAGGTCCGTGATGGGATAATGCAACTTGAGAAG
GTGTATCTTTTTAAGAATTTCAAACATGCCTTAGCCTTCACGAATGCAGT
GGGGGAAATATCAGAGGCCGAGGGGCACCACCCGGGCCTTCTTACTGAAT
GGGGTAAAGTTACTGTGACTTGGTGGAGTCACTCTATCAAAGGTCTGCAC
AGGAATGATTTTATTATGGCAGCCAGAACGGATGAGGTCGCGAAGACAGC
AGAAGGGCGTAAA

Seq. ID No: 36

>BH4syn_1ATGGAAGGGGGTCGTCTTGGCTGTGCTGTATGCGTACTAAC
TGGGGCCAGCAGAGGATTCGGCAGGGCCTTGGCTCCACAATTAGCTGGAC
TACTGTCCCCGGGATCTGTGCTGTTGCTTTCTGCGCGTTCCGACTCCATG
CTAAGACAGTTGAAAGAGGAGTTATGTACCCAGCAACCCGGTTTACAGGT
AGTTTTGGCTGCCGCCGACCTTGGTACTGAGTCCGGCGTGCAACAACTAC
TTTCTGCAGTCAGAGAATTACCCCGTCCAGAAAGACTTCAGAGGCTGCTG
TTGATAAACAATGCGGGCACACTGGGTGATGTGTCAAAGGGATTTCTAAA
TATCAATGATCTAGCAGAGGTGAATAACTATTGGGCCTTAAATTTAACCA
GCATGCTTTGTTTGACGACAGGGACATTAAACGCATTTTCTAACAGCCCC
GGACTTTCAAAGACAGTAGTCAATATTTCTTCACTGTGTGCGCTGCAGCC
CTTCAAGGGGTGGGGACTGTACTGTGCTGGTAAAGCAGCTCGTGACATGC
TGTACCAGGTGCTGGCCGTTGAAGAACCAAGTGTTAGAGTTTTAAGCTAC
GCCCCTGGACCACTAGACACGAACATGCAACAGTTGGCCCGTGAAACCTC
TATGGACCCAGAGCTGAGATCCAGGCTGCAAAAACTTAACTCTGAGGGAG
AGCTAGTTGATTGTGGAACTAGCGCACAAAAACTATTGTCCTTGTTACAA
AGGGATACCTTCCAAAGTGGAGCGCACGTCGATTTCTACGACATT

Seq. ID No: 37

>BH4syn_2ATGCACTCCCCGAGCCTGTCAGCGGAAGAAAATCTTAAAGT
CTTTGGAAAATGTAATAACCCTAACGGCCACGGTCACAACTACAAAGTTG
TGGTAACTATACACGGCGAGGAGGCAATCATGAAGCCTTTGGACCATAAA
AATTTGGACCTGGACGTACCTTATTTCGCTGACGTAGTCTCTACAACCGA
AAATGTAGCAGTATACATCTGGGAAAATTTGCAGAGGCTACTACCGGTGG
GAGCACTTTACAAAGTTAAAGTATATGAGACAGACAATAATATCGTAGTC
TATAAGGGTGAG

Seq. ID No: 38

>BH4syn_3ATGCCATCATTGTCCAAGGAGGCTGCCTTGGTTCACGAGGC
TTTGGTCGCAAGGGGGTTGGAAACGCCGTTGCGTCCTCCGGTTCACGAGA
TGGACAACGAGACTAGGAAATCACTAATCGCTGGTCACATGACTGAAATC
ATGCAATTGCTTAATCTTGACCTTGCTGATGATTCACTGATGGAGACTCC
ACACAGAATTGCGAAGATGTATGTGGACGAAATATTTTCCGGTCTTGACT
ATGCCAACTTCCCGAAAATTACCTTGATTGAAAACAAAATGAAAGTGGAC
GAGATGGTGACCGTTAGAGACATCACCCTTACCAGCACTTGCGAACACCA
CTTTGTGACTATTGATGGAAAAGCCACCGTGGCGTATATCCCAAAGGACA
GTGTCATCGGGTTGTCAAAAATCAATAGAATAGTCCAGTTCTTTGCACAA
AGGCCACAAGTTCAGGAACGTCTTACTCAACAAATCTTAATCGCCCTTCA
AACCCTATTGGGGACCAATAATGTCGCTGTGAGCATCGATGCGGTACACT
ATTGTGTTAAGGCCCGTGGTATTCGTGATGCGACGAGCGCCACCACGACG
ACCTCACTGGGAGGACTGTTTAAGAGTTCACAAAATACTCGTCACGAGTT
TTTGAGAGCCGTTAGGCACCATAAC

Seq. ID No: 39

>BH4syn_4ATGCACCACCATCATCATCATACGTCCAGTACACCTGTAAG
AACCGCTTATGTCACGCGTATAGAACATTTTAGCGCGGCCCATAGGTTGA
ATAGTGTCCACTTGAGTCCAGCTGAGAACGTAAAACTGTTTGGCAAGTGC
AATCATACCTCCGGGCATGGTCATAACTACAAAGTAGAAGTAACGATTAA
AGGACAGATCAACCCACAGAGCGGGATGGTGATCAACATAACCGACTTAA
AGAAGACCTTGCAGGTGGCCGTTATGGACCCGTGCGACCATAGAAACCTG
GACATTGATGTGCCATATTTCGAGTCCCGTCCATCAACCACTGAGAACTT
AGCAGTTTTTCTCTGGGAAAACATAAAGTCCCACTTACCTCCTAGTGATG
CCTACGACCTATACGAAATTAAATTACATGAAACCGACAAAAACGTGGTG
GTCTACAGAGGCGAG

Seq. ID No: 40

>BH4syn_5ATGCATCATCACCATCACCATAGCTCCAAGGAGCATCACTT
GGTCATAATCAACGGTGTCAATAGAGGGTTTGGGCATTCAGTTGCCTTAG
ACTACATTAGGCATTCTGGGGCGCATGCCGTCAGCTTCGTGCTTGTAGGG
CGTACGCAACACTCTTTAGAACAAGTGCTAACTGAGTTGCATGAAGCAGC
ATCCCATGCTGGAGTGGTCTTCAAGGGCGTCGTTGTTAGTGAGGTCGATT
TAGCACATTTAAATAGCTTAGACTCTAATCTAGCCCGTATTCAGTCTGCC
GCAGCAGACCTAAGGGATGAGGCGGCGCAGAGCACTAGAACAATAACGAA
GAGCGTGTTGTTTAACAACGCCGGTTCACTAGGGGACCTGTCAAAGACTG
TAAAAGAGTTCACCTGGCAAGAGGCAAGGTCATATCTGGACTTCAACGTG
GTCTCACTTGTGGGACTATGCTCTATGTTTCTTAAAGACACGTTAGAAGC
GTTTCCAAAAGAACAATATCCCGACCACAGAACTGTCGTTGTCTCCATTT
CTTCCCTGTTGGCTGTCCAGGCGTTCCCAAATTGGGGGCTATACGCTGCT
GGGAAAGCGGCCCGTGATAGATTGTTAGGCGTCATAGCGTTAGAGGAAGC
CGCCAATAATGTTAAGACTTTAAATTACGCGCCGGGTCCCTTAGACAATG
AAATGCAGGCCGATGTCAGACGTACTCTTGGCGATAAAGAGCAGTTAAAA
ATTTACGATGACATGCATAAATCCGGCTCTCTAGTTAAGATGGAAGACTC
AAGTAGAAAACTTATTCATTTACTAAAAGCTGATACGTTCACGTCTGGCG
GGCATATAGACTTTTACGACGAG

Seq. ID No: 41

>DAC_1ATGGTTGATGCAGACATAGCCCTGAATTGGGCGGGAGGACTTCA
TGTATGTATCGTGAGACCTCCGGGACATCATGCAGAGCCCGGCGCTGCAT
GCGGTTTTTGTTTCTTCAACAATGTCGCTCTAGCTGCGAGATACGCTCAG
AGCTTGCAGAGTCCTTCCGACCCACCCCTGAGAGTCATGATTTTAGATTG
GGATATTCATCACGGTAATGGCACCCAACATATCTTCCAGGATGACGCTA
GTGTGCTATATGTATCACTTCATCGTTATGACGACGGCACATTTTTCCCG
TCCTCAGAAGACGCTGCGCATGATAAAGTTGGAAGCGGACCGGGCGAGGG
TTTTAATGTAAACATCCCCTGGAATGGGGGTAAAATGGGGGACGTGGAGT
ACTTGCTGGCTTTTCACAGAATAGTGATGCCTATTGCTTACGAATTTAAC
CCTCAACTGGTATTGGTGTCCGCGGGCTTTGACGCGGCTAGAGGTGATCC
TTTAGGGGGCTGCCGTGTCTCACCTGAAGGGTATGCACACATGACCCATT
TGCTAATGGGTTTGGCGGGAGGAAAAGTAGTCGTTGTACTTGAGGGTGGG
TACAATCTAACATCAATAAGCGAGTCAATGAGCATGTGTACGAGAACACT
ATTGGGAGATCCTTTGCCTTTCATATCAGATTTGCATGCCCCTAGACCAG
CCGCGTTGAGAGCGATCTCTAGTGTCCTGGGGGTCCATCAGAAGTATTGG
AGAAGTTTGTGCATAAACGTAGGGCCGCCA

Seq. ID No: 42

>DAC_2ATGAAAACACATCCACACCCGGAACGTCCAGATCGTTTGCAGGC
CATAGCAGCGTCCTTGGCCACTGCCGGCATATTTCCCGGAAGATGCTACC
CGATCCCGGCGAGAGAGATTACCAAAGAAGAATTGCAGATGGTTCACTCA
CTGGAGCACATTGAAACTGTCGAGCTTACGGGCCAAATTCTATATTCTTA
TTTTACTCCTGATGGCACGAATCCACATAATAGGCTGAAGCTGGATAACA
GGAAGTTGGCGGGCATCCTATCACAGAGGATGTTTGTAATTCTACCATGT
GGAGGACTGGGAGTTGATAGCGATACTATCTGGAATGACCTTCATTCCTC
CAACGCTGCCAGATGGGCCGCCGGTTCAGTTATTGACTTAGCTTTCAAAG
TGGTCACCAGAGAATTGAAAAACGGCTTCGCTCTAGTTCGTCCGCCCGGG
CATCATGCAGATCCATCCACCGCAATGGGGTTTTGCTTCTTCAACTCAGT
GGCTATTGCAGCTAAACAGCTTCAGCAGAAACTAAACGTGAGGAAAATTC
TGATTGTGGATTGGGACGTGCACCACGGGAATGGTACGCAGCGTGTATTT
TATCGTGACCCTAACGTGCTTTACATATCCTTACATAGGCATGACGACGG
AAACTTCTTCCCAGGGTCAGGAGCAGCAGATGAAGTAGGTGCCAATAGCG
GGGAAGGCTTCAACGTTAATGTTGCATGGGCAGGCGGCCTAGACCCGCCT
ATGGGTGATGCGGAATATCTAGCAGCGTTTAGGACAGTTGTCATGCCGAT
AGCCCACGAGTTCGCCCCCGATGTAGTACTTGTGAGTGCGGGGTTCGACG
CTGCGGAGGGT

Seq. ID No: 43

>DAC_3ATGATGGCAACAGAACCGATAGCGAGTGGGAGCGGCACAATGGA
CATCGACAGCGAGAAGACGCCAAGCACGAGCCAGGCCAATCCAATGGCAG
ATACCTTTCAGACCCGTGAAGCGGTACTAGGATTAGGTGAAGTAGTGGAG
CATGTAGGCGGGCGTTGGGTGGCGGAGCAAGAGTGGATTAGAAGTCCTGA
GAGGAAAATGGCATACACTCAAGGTACTAAAAAGAAGGTGTGCTATTACT
ACGATGGAGACGTCGGCAATTATTACTATGGACAGGGTCATCCTATGAAG
CCCCACAGGATCAGGATGACCCATAACCTGCTGTTAAATTACGGATTATA
CAGAAAAATGGAGATTTACCGTCCCCACAAGGCCAACGCTGAGGAGATGA
CTAAATATCACTCTGACGACTACATAAAGTTCCTAAGAAGTATTCGTCCA
GATAATATGTCAGAGTATAGCAAGCAGATGCAGCGTTTTAACGTTGGTGA
AGACTGCCCAGTATTCGACGGTCTATTTGAGTTTTGTCAGCTGTCCACGG
GCGGGAGTGTAGCGTCAAGCGTCAAATTGAACAAACAGCAAACGGACATA
GCCGTGAACTGGGCCGGCGGATTACACCACGCGAAGAAGTCAGAAGCGTC
TGGATTCTGTTACGTAAACGACATCGTACTAGCGATCCTTGAGCTTCTAA
AGTATCACCAGAGAGTTTTATATATTGATATAGACATCCATCACGGCGAT
GGGGTGGAAGAAGCGTTTTATACGACGGATCGTGTAATGACGGTATCATT
TCACAAATATGGGGAGTATTTCCCAGGAACAGGTGACTTGAGAGACATTG
GTGCGGGTAAGGGTAAATACTACGCGGTCAACTATCCCCTTAGAGACGGC
ATAGACGACGAGTCC

Seq. ID No: 44

>DAC_4ATGGACGCGGGTACGCGTAGAGTGGATGATGCGGCCGTTCCAAG
TACGGGTCCATCCGCATCCTTACTAAGATCAGCGAATATGTTAAGTGCCG
CGTTCGGTCTAACAGCATCTCTATACTCCAGATTACGTGGGGTGTGCTCC
AGTAGAAGAGCCCTATCCACGTCAGCTCGTACGTCTGAGGCGGCCGGAGT
AGGGGCGAAACCCGGAGTCGCTGCCGCATTAACCGTTCCCTCAACTGGGC
CCTCTGCTTCCGAAGCATCACCAGCTGCTCTATTAAGGATTCAAGTCGCG
GAAGAGTGGGCGCGTGCCAGTGGACTATTGGATAGGGAAGACTGCCAGGT
TGGTCTGGCATTCGATGAAGCCATGCATTTACATAGTGGACCAGCTGGTC
ATCCTGAAAGACCAGCCAGGACCAAAGAAATACTGGCGCAGCTACACGCG
TCAGGTTTAGTGAGAGCTTGTGCCCAAGTCCCTAGTAGGGAAGCGACTGA
AGAAGAATTACTATTGGTCCATGACGCCAGACATGTAGAGAGGGTGTTAC
GTCATGAAGCGGCTGGTCATAAAAAGGCTAAGGCTTTCAGTTTCCCTTTC
GGCCCCGATACATATGTTTGCGAGCATACAGCGAGATGCGCTCGTCTAGC
TGTAGGTTGCTTATTGTCTCTTGTTGACGCTTCTTTAGATCCAGCAAGTC
CGGTTCGTACAGGGATGGCGGTAGTTAGACCGCCCGGGCACCACGCAACC
AGTGATCGTGCGTCCGGATTCTGCTTGTTTAATAATGTCGCTGTTGCCGC
CAGACACTTGCAGAGACGTCATGGTTTAAAAAGAGTTGCAATTGTTGACT
GGGATGTTCATCACGGTAACGGTACGAACGATCTGTTTACCGAAGACCCA
AATATACTGTTTTTCTCCGTTCACCGTTTTGATAATCACGGGTTTTTCCC
CGGGAGTGGTTTTCTTGAGGATGTTGGTCATGCGCAAGCTCGTGGTTACA
CGGTGAACGTCCCCCTGGAGAAAGGCTATGGGGATCTTGATATCGTTCAT
GTCGTAAAATACGTGCTTTGCCCCGTCCTAGAGAGATTCAAGCCCGATGC
GATCCTGGTCTCCGCTGGGTTCGATGCCGTAAAGGGCGATCCACTAGGCG
AGTGCAGAGTGTCTCCGGAGGCTTTTGGCTGGATGACGCGTTGTCTACAT
CGTTTAGCTCAGCGTTATTGCGACGGCAGGTTGTTCCTGGTACTAGAGGG
CGGGTATAATCCTGACATGATAGCACAGTGTTGCATCGAATGCGTTCAGT
CCCTAGTGGCAGAGGCTGCTGGATTAAGGGGCCCTTGGCCCGAGTTCCCT
GCTGTGGGAGTTCCGTTGGCAGAAGGAGCGCAGCTGAGTGCACCCAGCTC
AGCCCCTACCTCTGCCCCCGGGACGCCAACTTCAACAAGCCCAGCCAGCT
CCCCAGCCCTGTCCGCCGCAGCACCACCTTTAGCTTCCCCCGGTAGCACA
CCAACTTCCTCTCCTTGTTTAAGGCCAAGTGGGGGTGAAGCACCGCCTAG
ATCACCTCCGTCTGCCAGCGCGTCAGCGGGAGGAGGCGCAAGGCAAAGGG
CGCGTGCTCCAAGTTCTAAGACAGTCAGGGCGGTAAGACAGCTGACAGAG
ATACATCACCTGCTACCTTTGGAGCTGCCTGTTGCGCCGCGTCCAGGGGA
CGGTCCCGGCGCTGCAAATAAGTCCGCCAGAAAGAATGAACGTAGGAGAC
TTGGGAGAGGTAGGAGGGGACCGGAAGAGGAGGGAGCCAGTAGTGACAGT
TCAGGCTGGGCCATCGCTTGTGGTTTAAGTGATGCTGAACCCTGGCCGAG
TCCACAGGCGTCCCCTGTGGCTTCGCTTTCCCAAGGAGCTTCTTCCTTAC
CCACGTTAGAATTACCGCCCGCGTTTCCAAGTTTAGACGGTGTTGGAAGT
ACGGCCGGGAACTCTTACCTGGGTACGTCAGGGAACGTGGGAATCGACGC
GGCCGGCCATAGTGCATCTTCATGGCTAGGTTCTCCGACTACAGCAGCGA
CCGCAGTCGCCCCTCCCGCGAGGGGCGACAGGAAGGTGAAACGTCGT

Seq. ID No: 45

>DAC_5ATGGTGGATGCCGACATCGCGCTTAATTGGGCGGGAGGACTGCA
TGGTCATCACGCAGGGCGTGGATGTAGCGAAGGATTCTGTTTGTTGAACA
ACGTAGCAGTGGCCGCTGCCTACGCACGTAGTGCACATCCCGAACAGGTC
AGAAGAGTTTTGGTGCTAGACTGGGACGTTCATCATGGCCAGGGCACTCA
AGAAATCTTTTGGAGGGACCCCGGAGTGTTGTATGCGAGCGTCCATAGGG
ATGGTGGGGAGGGATTCTATCCGGGAACCGGGGCCGCTGAGCAAGTTGGA
GACGGAGCGGGGAGGGGCTTTACCGTGAACGTACCTCTTCCGACGGGCTA
TGGTGACGGCTGTCTTTGGGCTGCTTGCGCCGAAGTTTTGTTACCAGCGG
CAAGAAGATTCAGGCCGGATCTTATACTTGTATCCGCAGGATTCGACGCG
GTTGCAGGAGATCCTTTGGGCGGGTGCCGTTGTACTGCCCGTGGCTTCGG
AGCACTGACGGGTGAATTAAGAAAGCTGGCTGGGGAACTGTGTAGCGGTA
GACTGCTTCTAGCCTTGGAAGGAGGCTACGATTTAAGGACGTTGAGAGCT
TGCGTTGGGGAAGTGTGCCAAGCACTAGCAGCTCCGGAACCGGCAGAAGG
TGGAGCC

Seq. ID No: 46

>DAC_6ATGAGGAATAGGTCAAGTGGGTTCTGTCTTGTAAACAATGTCGC
TGTGGCAGCCGAATATGCAAGAGATCGTTACCCTGAAGTGGAGCGTGTTC
TAATCTTCGACTGGGACGTTCATCACGGCCAGGGAACGCAGCAGATCTTC
GAGCAGTCCCCAGATGTACTGGTAATCTCAGTGCACAGGCACGATGGTCA
CAGCTTCTATCCTGCAACTGGAAGTGCAGGGGAAGTAGGCTCTGGGCCGG
GAAGAGGGTATTCTGTCAATGTGGCTCTTCCTGCAGGTTATGGCGGGGCT
GCACTTTGGACAGCTTGTGCCCATGTCCTTCTGCCTGCGGCAAGAAATTT
TCAACCCCAACTTATTCTAGTCTCCGCTGGCTTCGATGCAGCGGCGAGTG
ATCCACTAGGGGGATGTTTCGTCGAACCCCGTGTCTTTGGTGCATTAACA
GCGGAATTAAGGAGGCTGGCTGCAGAGGTTGCGGAGGGGAGACTTATCCT
GGCCTTAGAGGGTGGCTACAACCCGGAGGTGCTTGCAGATTGTGTTGATG
AGGTCGCCGCCGCGTTGGTTGCAGATGCCTCAAGTTCCGGTGTAGAAGCA
TTCGCCGAGGCCCCATCATGGCTCGCAGGGTCTGCATGCTTCGGGGCGAT
AAGAAGGACTTGTGAGGCACATAGGATGGCTCCCTTGAGATTACCTCTTC
CGTCATCAAGAATCGACAGAAGAAGAGCTGCTGCTCGTCAGGCGGAAGCG
TTGTCTAGCCCGTCTAGCAGAGACGCTGGAGATACGGGAGGCGGCGAGGT
ATCTGCACACGGCGCCACAACAACGGTTACCACATCAGCAAATTTGGGAG
CTGGTACTCTAGCGGCGAGGCCATCCAGTATGGTGACAGGCGAGGGCAGG
AGAGCCAATGGCCAGCTAGTGGACGTTCTGGGTATCGCACTTGCTGGTAA
GCCTTCAGCATCTCCGTGGCCGGAGGCTCAGCGTACACAGGGATCTGCCC
CCGGTACCCCGGCGCCCGCGACAGGGGGCGCGTTACCACCAGCTGAAACG
GCCGAATCACCTGGGTCCGTCGCAAGCGGAGCCGCGGTTGCCCAGGGTCC
AGTAGAATGTCAAGCGGCGGCACGTCAAGCTGGAGAGTGCCCGGGGCAAG
CCCCGGCTCCGGCAGGAGCGGGAGCAGCGCCTGGAGGAAGGGGTGTCGAA
GCGGCAGCCGCGCAGCACGGACAGGATTTGGCTCCAGCGGCAGGACCCGG
AGCCGCCGCATTAGTGGAACTTCAAACGGGGGAGTTGGTCGTAAGAATCG
CACCCCTTCCAAGGCCAAAAGACGTTGTGGTTTCCGCAGAGGAGCTGTGG
GTTTGGCATGATCAAGGTGGGCCGCTGGGCGTGCAGAGGTGGAGGTTTGA
AGGCGTACGTGCCGAGAACTCCGGAGCACTCAGATGTGCCGAATTTAGGT
CCAAAAGGCACGAGTTGACCGTAAGACTAAGGTTGGGT

Seq. ID No: 47

>DAC_7ATGGTGAGAAGTAGTCAGGCTACAACATTTAGTTCTTCCCCCTA
CTTTGCGGATAGGGGGATTCACGGTACCGCACAGCCGATCACTGAAAGAA
TAAACCCGCGTAAGTGTACTTTTCACATATTGCCACCAGGCCTTGGGGGA
TGGTTGTATCTTTTTCCAAAAATGAGTAAAACAGTTGCTTATTTTTATGA
TCCGGACGTCGGCAACTTTCACTACGGCACAGGACATCCGATGAAGCCGC
ACAGGCTGGCGCTTACACATAGCTTGGTTCTGCACTATGGGTTATACAAG
AAGATGATTGTCTTCAAACCATACCAGGCAAGTCAGCATGACATGTGCAG
ATTCCACTCAGAAGATTATATTGACTTCCTGCAACGTGTAAGCCCAAATA
ATATGCAGGGATTCACGAAGAGCTTAAACGCGTTCAACGTAGGAGACGAC
TGCCCTGTGTTTCCGGGGTTGTTTGAGTTTTGCAGTCGTTATACCGGCGC
ATCTTTGCAGGGAGCCACGCAACTAAACAACAAAATTTGCGATATAGCGA
TTAACTGGGCAGGGGGCCTGCATCATGCGAAGAAGTTTGAGGCCTCAGGT
TTCTGTTATGTGAATGATATAGTCATCGGGATTCTTGAGTTGTTGAAATA
CCACCCTAGAGTCCTATACATTGATATTGACATTCATCATGGAGACGGTG
TTCAAGAGGCATTCTACCTAACTGACAGAGTAATGACCGTAAGTTTCCAC
AAATACGGAAATTACTTTTTCCCAGGAACAGGAGATATGTACGAGGTCGG
CGCAGAATCAGGGAGATACTACTGTCTTAATGTTCCTCTTAGGGATGGAA
TTGATGATCAGAGCTATCGTCACTTGTTTCAGCCAGTAATCAAACAGGTC
GTTGATTTCTATCAGCCAACTTGCATTGTTCTTCAGTGCGGGGCGGATTC
TCTGGGATGTGACCGTTTGGGTTGCTTCAACTTATCCATACGTGGGCATG
GGGATTGTGTACAATATGTTAAATCTTTCAATATACCACTGCTAGTATTG
GGCGGTGGTGGGTACACAGTTAGGAACGTCGCCCGTTGCTGGACATACGA
GACGTCACTTCTAGTGGACGAAACGATATCTGAGGAGCTACCATACAGTG
AGTACTTCGAGTATTTTGCTCCGGATTTTACGCTACATCCAGACGTTAGT
ACAAGGATTGAGAACCAGAATACGCGTCAATATCTTGACCAAATTAGGCA
AACAATCTTCGAGAATCTGAAAATGTTGAATCACGCCCCCTCCGTTCAGA
TCCATGACGTGCCGAGCGATTTGCTTTCCTACGACAGGACAGATGAGCCG
GACCCCGAAGAACGTGGGGCCGAGGATAATTATACGAGACCAGAGGCATC
TAACGAGTTTTACGATGGAGACCATGATAATGATAAGGAAAGTGATGTGG
AGATC

Seq. ID No: 48

>DAC_8ATGTGGGATGTTCATCATGGTCAGGGGATACAGTACATTTTTGA
AGATGACCCTAGCGTGCTATATTTTAGTTGGCATAGGTATGAGCACGGTT
CCTTCTGGCCCAATCTTTCAGAAAGCGATTACGATAGCATAGGGAAGGGT
AGAGGGACGGGTTTTAACATCAACCTACCCTGGAATCAGACTGGGATGGG
TAATGCGGATTACGTGGCCGCCTTTTTCCATGTATTGTTGCCACTTGCCT
TTGAGTTCAACCCAGAGTTAGTCCTTGTTTCTGCCGGATATGATTCTGGG
ATCGGTGACCCCGAGGGACATATGAGAGCTACACCTGAGTGTTTTTCTCA
CCTAACTCACATGTTGATGCACCTTGCAGGGGGCAAGCTGTGCATGATAT
TAGAAGGTGGCTACCACCTACGTAGTTTGTCCGAGAGCGTAAGTATGACC
GTAAGGACTTTGCTACGTGATCCAGTGCCCAGGCTTTCAGGTGAAATGAC
GCCCTGCTATTCAGCCCTTGAGTCTATACAGAATACGCGTCATGCTCACT
CACCATATTGGAAATGCCTTCTTCACGACGAAACTAGACTGGTGGAAGAG
ATTTCTACGAAAGGACTGAAGGCACCGGGGCCGTTGCATGTTGATGCAAG
CGTGGTTGATGAATTTCTTGAAAACCACATGAAGAAGATCTTGCACCCTA
CTCCCCCAATTACAACTATGGTCGTCGCAAGCGTGGAGAATACGCTTAAC
TTACCCGCGGGTGTTCAGTTAGAGGAGTCCACTGTCACGCCAGAGCAGGC
CAGACATGCAATCTCCGTATTCAACCCAGATGAGTTAAATGAGAACGTGC
TTAACTCAGTTTCAAAGATGCTTCCCGCCCTAGAGAAACTCGTCAAT

Seq. ID No: 49

>DAC_9ATGAAAACCCATCCCCATCCTGAACGTCCGGATCGTCTTCAAGC
GATTGCCGCAAGTTTGGCTACAGCAGGAATATTCCCAGGGAGATGTTACC
CTATCCCGGCAAGAGAGATCACTAAGGAAGAGTTACAGATGGTACATAGT
TTGGAGCATATAGAGACGGTCGAATTGACAGGTCAGATTCTATATAGCTA
TTTCACGCCGGACACCTATGCGAATCAACACTCAGCTCACGCTGCGAGGC
TTGCGGCGGGTTTGTGCGCTGACCTAGCTAAAGAGGTCTTTAGTGGCAGG
GCTAAGAACGGCTTCGCATTGGTTAGACCGCCAGGGCACCATGCTGGTGT
CAGGCAGGCAATGGGATTTTGCCTACATAACAATGCGGCGGTAGCGGCGC
TTGCCGCCCAAGTGGCGGGAGCCAAGAAGATCTTGATAGTAGACTGGGAC
GTTCATCATGGGAACGGCACACAGGAAATATTCGAGCAAAATAAATCTGT
TTTATACATATCGTTACACAGGCACGAGGGCGGAAAGTTCTACCCGGGTA
CAGGGGCGGCGCACGAATTTGGTACTATGGGAGCCGAGGGCTATTGCGTG
AATATTCCTTGGAGTCGTGGTGGCGTCGGCGACAATGATTATATATTTGC
GTTCCAGCATGTTGTCTTGCCTATAGCGTCCGATTTCGCTCCTGATTTCA
CGATAATATCCGCCGGCTTTGACGCTGCAAGGGGTGATCCACTAGGGTGC
TGCGACGTGACTCCCGCTGGTTACGCTCAGATGACGCATATGTTAAACAT
TTTAAGCGGGGGCAAACTACTTGTAATTTTGGAGGGTGGATACAACCTAC
GTTCTATCTCAAGTTCAGCAACAGCAGTAATCAAGGTGTTGCTGGGTGAA
TCTCCCGGCTGCAATCCCAAAAATTTCCTTCCGTCCAAGGCGGGTGTTCA
AACTGTCCTAGAGGTCTTGAAGATACAAATGAACTTCTGGCCGGCTCTAG
GCTCAATTTATAGTGACCTACAAACGCAATGGGGAATGTACTGCATGAAG
ACCAAAAAGAAGCAAATAAAGAAAAGACAACGTGCGGCGGCGCCGCTTTG
GTGGAAGTGGGGGCAGAAAAGTTTCTTATATCACTTGCTTAATGGTCATC
TGCATGTAAAAAGCAAGGGCTGC

Seq. ID No: 50

>DAC_10ATGTTTCTAGTCAGACACCACTTATATAAATGGAAATCACATT
TTCGTATCGATGCCGACGGTAAATTCGTCGAAGATCAGTTCTTTCCAAAG
AACTTGAAAAGCGGCAGACGTTTCTTACGTAGCATAGGCGCTTCTATCAC
GTGCTCTAATGGCATTGGAAAAGATCCATATATTTTGAGCAATGAGAAAA
TTTCAGACGCTAGGCTAATATACGCAGTCGCGCCAGCGATGGGTCATAAT
CAGGAAAGCCACCCTGAGAGTCACTTTAGGGTTCCTGCCATAGTCAACGC
CTTAGAGAAGATGGAAATGACGCCTAAATTCAGGGGTTCCGAGATTATTG
AGTTGCAATCTTTTAAGCCCGCTTTAGTTGATGATATAGCGTCTGTCCAT
GCTAGAGCATATGTATCCGGTTTGGAAAAAGCGATGGACCAGGCGAGCCA
ACAGGGTATAATATTTATTGATGGCTCAGGTCCAACATACGCAACGGCTA
CGACATTTCACGAGTCTCTTGTGGCGGCAGGGGCGGGGATAGCATTGGTC
GATTCTGTGGTTGCGGCTTCTAAAAACCATTTAGACCCGCCAGTGGGTTT
TGCGCTAATAAGGCCCCCGGGCCACCACGCCATACCACTGGGCCCGATGG
GGTTCTGTGTGTTTGGGAATGTCGCTATTGCCGCAAGATATGCTCAGCGT
GCCCACGGGCTAAAAAGGGTTTTTATAATTGATTTCGATGTGCACCACGG
CAACGGAACCAATGATGCGTTTTATGATGACCCAGATATATTTTTCCTAT
CCACTCACCAAGACGGGTCTTATCCCGGCACGGGGAAGGTGGATGAAGTA
GGAAGGGGCGATGGAGAGGGTACCACACTGAACCTGCCATTGCCCGGGGG
TAGTGGCGACATAGCTATGACAACAGTATTCGACGAAGTCATTGCTCCAT
GCGCTCAACGTTTTAAACCCGACATAATTTTGGTTTCTGCAGGCTATGAT
GGGCATGTATTAGATCCATTAGCAAGCCTTCAATTTACAACTGGCACGTA
CTACATGTTAGCCAGTAAAATTAAGTTGCTGGCGAAAGATTTATGCGGCG
GTAGGTGTGTATTCTTTCTGGAGGGCGGTTATAACCTGGAGTCTCTATCC
TATAGCGTAGCGGATAGCTTCAGGGCATTTTTAGGAGAACAGTCCTTGGC
ATCAGAGTTTGATAACCCAGCGATCTTGTACGAAGAGCCATCTACGAAAG
TAAAACAAGCTATCCAGAGGGTTAAGCATATTCACTCCTTA

Seq. ID No: 51

>DAC_11ATGGTGGACGCTGACATCGCGTTGAACTGGGCCGGAGGATTGC
ACCACGCCAAGAAATCTGAGGCGAGTGGGTTTTGTTATGTGAATGACATC
GTCTTAGGAATCCTAGAACTACTGAAAGTACACAGGAGAGTCCTATACGT
TGACATAGATGTGCACCACGGTGACGGCGTGGAAGAGGCATTCTATGCTA
CAGATAGAGTAATGACTGTTTCTTTTCACAAGTTCGGCGATTTCTTCCCC
GGAACGGGCCATATTAAGGATACCGGGTGGGGCCCCGGAAAGAACTACGC
TCTTAATGTCCCGCTTAACGATGGAATGGACGATGAATCATTTAGGGGAC
TGTTTAGACCCATCATACAGGGGGTGATGGAAGTATACCAGCCGGACGCT
GTCGTATTACAATGCGGCGCTGATTCTCTATCCGGGGATAGGCTAGGTTG
TTTTAACCTTTCAGTCAAAGGACATGCTGATTGTCTTCGTTTCTTGAGGT
CTTTTAACGTCCCACTAATGGTTTTAGGCGGGGGTGGTTATACCATCAGG
AACGTCGCTAGATGCTGGTGTTACGAGACAGCTGTAGCCGTCGGAGTAGA
ACCAGATAACAAGCTGCCGTATAACGAATACTATGAATATTTCGGCCCCG
ATTATACCCTTCATATTGAACCATGTAATATGGAAAATCTTAATACCCCC
AAGGATATGGAGAAGATAAGGAACATGCTGCTGGAACAGCTAAGCAGGAT
ACCTCATGTGCCGAGCGTTCCATTCCAAACCACGCCGCCAACAACCCAGG
TCCCCGAAGAGGCCGAGGAGGATATGGACAGAAGGCCAAAATGCAGAATC
TGGAATGGGGAGGACTACGATTCTGATCCAGACGAAGATGAGAAGCCTAG
ACATACGGAACCGAATAGCGAGCTTCGTGATGTAGTGGACGAAATGGACG
AGGACAAACGTGAAGAGCACCCACCCTCA

Seq. ID No: 52

>DAC_12ATGTGTTCAGACGCAAATGGGAAGGTTGGTAACATTTCTGTGA
TGAGCACTGAGGGAATATCACAGGTGGAGAGTAAAAAGGCTCGTCTTAAT
GGCTTACTTACACTAGAAGATATCTATAATCTGCCAGACGAGCTAGATGA
TGATGAAGACGACAGTGACTGGGAGCCTTTACTTGAACCTCTTGCGGTAC
GTAAATGGTTTTGCACCAACTGCACGATGGTCAATTTTGATGGATTTGAC
TTCTGCGAGACGTGCGAGGAGCATAAGGAAAGTGGCATCCTGAAACAAGG
CTTCTTCGCCTCTCCTGCCTTACAAGGCACAAGAAGCACTCAAATCGAGT
CTGAGGTTATCGAGAGATATACTGAGTCTATATGTGACATCTCAGCTTCT
GCGCTTAGTACCGTGGTCGGTTTCGACGAGAGGATGCTACTACATAGCGA
AGTGGTCTTAAAACCACATCCTCACCCACATCCAGAAAGACCGGATAGAC
TGAGAGCAATTGCGGCATCACTGTCAACCGCAGGGATCTTCCCTGGCAAA
TGCCATCCAATCGCCGCGAGAGAAATCACCCAAGAGGAACTGTTGAAAGT
TCACTCACTTGAGCATGTCGAGGCAGTTGAAGTTACACGTCAGATGCTAA
GCTCATACTTTACCCCAGATACGTACGCTAACGAACACAGTGCGCAAGCC
GCGCGTTTGGCGGCCGGCCTTTGTGCAGATCTAGCATCTGAAATCTATTC
CGGAAGGGCCAAAAATGGATTCGCCCTGATTAGGCCCCCTGGACACCACG
CGGGTGTACACCAGAGCATGGGTTTCTGTCTTCACAACAACGCGGCGGTT
GCCGCATTAGCCGCACAAGTAGCTGGTGCCAAAAAGGTCCTGATTGTGGA
TTGGGATGTCCATCACGGTAACGGCACACAAGAGATTTTTGAAAGGAACA
AGAGCGTTTTATATGTCAGTCTGCACAGGCACGAAGCGGGCAAATTCTAT
CCAGGAACCGGAGCCGCCCATGAAGTTGGTACTATGGGTGCTGAAGGTTA
TTGCGTTAACGTACCTTGGTCCAGGGGGAGGGTTGGCGACAACGACTACA
TCTTCGCGTTCCAAAATGTTGTGATCCCAATTGCACACGAGTTTTCCCCA
GACTTCATTATCATATCTGCCGGTTTTGACGCAGCTCGTGGAGATCCTTT
GGGTGGATGCGATGTGACCCCGGCTGGCTATGCCTGTATGACTCATATGC
TATCTGCGCTCGCTGGAGGGAAAATGTTAGTTATCCTTGAAGGTGGGTAT
AACTTACGTAGCATCAGTTCATCCGCCACGGCGGTGATTAAGGTATTACT
AGGTGAAAAGCCGAAGTGCCAGTTTGAGAACATAGAGCCTTCCGCAAGCG
GGCTTCAGGCTCTTCTTGAGGTCCTGAAGGTACAGACTAACTTCTGGCCC
TGTCTGAGTTCTAAACTGACACAACTACAAAGCTGCTGGGAAGCATACTT
GTCTGGTCGTAAAAAGCAGAAGAAACGTAGGTTTCGTACTGTTGCTCCGC
CACCAATATGGTGGGCCTGGGGACGTAAAAGGTTCCTATACTTTTTAAGA
TGTCAAAGGTTCAGGATGAAACCA

Seq. ID No: 53

>DAC_13ATGGCTGGAGCAGAAGAGCTGCACGTGTTTTGGGAAGAAGGTA
TGTTAAAACATGAGACGGGACGTGGTGTTTTTGACACTGGGAGCGATCCG
GGTTTCCTGGACGTGTTAGAAAAACACCCAGAGAATGCAGATAGAGTTAG
GAACATGGTAAGCATTTTAAAAAGGGGGCCAATCGCCCCCTTCGTGAGTT
GGCACCAGGGAAGACCGGCTAGTCTGCCCGAGCTTCTTAGTTTCCATTCC
AGCGAATACATAGAGGAGCTAGAAGAGGCGGACAGGGCGGGAGGTAAGAT
GATGTGTTGTGGGACTTTCTTAAACCCAGGCTCCTGGAACGCTGCACTAT
TGGCGGCCGGAACTACACTGTCTGCGGTAAAGTATATTTTGGATGGCCAC
GGTAAGATAGCGTATGCCCTTGTCCGTCCCCCTGGTCACCACGCCCAGCC
GACCCAGGCCGACGGATACTGTTTCTTGAACAACGCTGGCTTGGCAGTGC
AGCTTGCCCTTGACGAGGGTTGCCGTAAAGTGGCCGTTATCGACATTGAT
GTCCATTACGGTAATGGCACGGCTGAGGGATTCTACTGTTCTAACAAAGT
TTTGACGATTAGCTTACACATGAACCACGGTAGCTGGGGTCCTAGTCACC
GTCAAAGTGGCACGCATGATGAGCTTGGGGATGGTGATGGGTTTGGATAT
AACATGAACATTCCCTTGCCCAATGGAAGTGGCGATAGAGCATATGAATA
TGCAATGCAGGAGTTAGTGGTGCCTGCTGTCCAAAAATTCGGCCCGGACA
TGATTGTACTGGTAGTCGGTCAAGATTCCAGTGCCTTCGACCCTAATGGG
CGTCAGTGTCTAACTATGGACGGTTACAGACAGGTTGCAAGGATTGTAAG
GGGCTTGGCAGATATGCATTGCAAGGGCAAGTTGTTGGTCGTACAGGAGG
GTGGGTATCATATTACGTACGCCGCCTACTGCCTACACGCGACTCTTGAG
GGAGCTTTAAACCTTCCTTCCCCACTATTGAGCGACCCGATAGCCTACTA
TCCAGAGGATGAAGGATTCGCAGTGAAGGTGATTGATGCGATGAAGGAAC
ATTATAAGAGTAACGTCCCTTTTCTGAAAGAGATCAAC

Seq. ID No: 54

>DAC_14ATGGGCTTTTGTATCTTTGGTAACATAGCGATCGCAGCCCGTT
ACGCCCAAAGAGTCCACGGATTGAAAAGGGTTTTCATCATCGACTTCGAC
GTTCATCACGGGAATGGCACCCAGGATGTCTTTTACGAGGACCCTGACAT
CTTCTTTTTGTCCACGCACAAAGAAGGCTCCTATCCAGGAACAGGGAAGA
TTCACGAAGTAGGGTGCGGTCCCGGTGAGGGAACTACTTTAAACCTGCCA
TTGCCCGGAGGGACTGGTGATGTCGCAATGAGAACAGTGTTTGACGAAGT
TATAGTCCCTTGCGCTCAGAGATTTAAACCAGATATTATTTTGGTGTCAG
CGGGTTACGACGCGCACTTTCTTGATCCTCTGGCGAATTTTCAGTTCAAA
ACGGCCACATATTATACTTTGGCTGCAAACATTAAACAATTAGCAAAAGA
ACTGTGCGGGGGCAGATGCGTGTTCTTCCTTGAGGGAGGTTATAATCTTA
AATCACTATCCTACAGTGTTGCGGATTCCTTTAGAGCCTTCTTGGGAGAG
CCGTCATGCGCCTCCGACGTTGATCCCACCTTTTTATACGATGAGCCGTC
AACTAAGATTGAGCAAGCAATCGATAAAGTGAAGGCCATTCACTCCTTA

Seq. ID No: 55

>DAC_15ATGGAACAATTGTGGGTTCCATCATTACCGATTTTAGGTGGGA
GAATCTTGCCTATGCTGAGGCACTATTGTGGTTTTGGTAGTCACCATCCG
CTAACATGGAGATCCTTACAGATTACTGGCCGTAAACAAAAACACAACGG
CTGCTGGATAGCCTATTGCCTGCCAAGCCATAATGGGACTTCAATAAGCG
ACACAAACGGGGTGAGGAAGGATTTAGCATTACCTGACAATCTTTTAAGA
GATGCGCACATCCTATATTGCACTTCTCCCGCTATGGGTCATAATAAAGA
AGCACACCCGGAGACGAATAAAAGAGTCCCGGCAATAGTGGACGCACTTG
AGAAGCTAGAGCTGACATCTAAACACAGAGGTTCACAGGTTTTAGAAATT
CAGGACTTCCAGCCAGCATCCCTTGACGATATAGCATTGGTCCACAGTAG
ATCATATATAACGGGACTGGAGAAGGCTATGTCTCGTGCCTCTGACGAAG
GACTGATCTTCATTGAGGGTACGGGGCCGACATACGCGACACAGACTACC
TTTCAGGAGTGCTTATTGTCTGCTGGTGCTGGCATAACGTTAGTGGACTC
TGTTGTCGCTGCTTCTAAGTTAGGCCCGAAGCCCCCACTTGGCTTTGCGT
TAGTTAGGCCGCCCGGTCACCACGCCGTTCCTGAGGGCCCGATGGGATTC
TGCGTCTTTGGTAATATAGCTGTAGCTGCAAGGTACGCGCAAAACCAACA
CGGCTTGAAGAGAGTCATGATAATAGACTTTGATGTACATCACGGGAATG
GCACCTGTGACGCCTTCTACGAAGATCCCGACATTTTCTTTCTTAGTACT
CACCAACTAGGCTCCTACCCTGGCACGGGGAAAATACACCAGGTCGGCCA
AGGAAACGGAGAGGGGACCACCTTAAACCTTCCATTACCTGGGGGAAGTG
GCGATTACGCAATGAGATGTGCGTTTGACGAAGTCATCGCACCCGCAGCC
CAGAGGTTCAAACCCGATATCATCCTAGTCAGCGCAGGATATGACGCTCA
CGCTCTGGACCCTCTGGCAGGCCTACAATTCACAACTGGCACCTTTTACA
TGCTGGCAGCACGGATTCGTGAGGTCGCTGCCGAACTATGCGGAGGTAGA
TGCGTTTTCTTCTTAGAGGGTGGATATAATTTGGAAAGTCTGAGCAGTAG
TGTGGCCGACACCTTCAGAGCGTTTCTGGGGGAGCCTAGCCTAGCCGCTC
GTTTCGATGATCCAGCAATGTTATATGAAGAGCCGACACGTAAAATCAGG
GAGGCTATCGACAAGGCTAAGCACCTACACAGTCTA

Seq. ID No: 56

>DAC_16ATGATGGCCACCGAACCGATTGCCTCCGGTTCAGGTACAATGG
ACATAGATTCAGAGAAAACGCCGTCTACCAGTCAAGCTAACCCTATGGCA
GATACGTTCCAAACAAGAAGACCAAGGGCTTCTTCCCTGCCACTTCAACC
ATCTAATTTAAAGGTAGGCTATATTTATAGCAGCGAGATGATGAATCACT
TCTGCCCTGGAGGGCATCCTGAACAACCTTTGCGTATTCAACAGATCTGG
GCGACAATCGTCAATGAGCAACTGCATAAAAGAATGAAGTGGATGCCGAT
CAGAGAAGTCAAGAAAGGTGAAGCACTGTTGGTCCATAGCGAGGATCACT
GGAACAAGGTCATTGCAATTCAGTACCTTACCGATCAGCAACGTGCAGAT
TCCGTTGATTATTACGAGCAAATGTCACTTTACGTGATGTCAGGCACGAC
TAGGAGTGCGCTACTAAGTTGTGGAGGGGTAGTCGAGGCATGTTTAGCTG
TTGCAAGGAACGAGTTGAAGAAGACCTTTGCCATTGTTCGTCCTCCAGGA
CACCACGCTGAACCCGATGAACATATGGGGTTTTGTTTCTTCAATAATGT
AGCCGTGGCGGCGCGTGTTGTCCAACAGAGGACTAAGTTAAAGAAGATAC
TAATACTAGATTGGGACGTTCATCACGGCAATGGTACACAGAGAGCCTTC
AACGATGACCCATCCGTTCTGTACATCAGTCTTCATAGGTACGAGCAAGG
GACATTTTACCCATGCGGTCCCTTCGGTTCTCTTACTTCTTGTGGTGAAG
GGCCGGGGACCGGTTTTTCCGTAAATGTGCCCTGGCCGTGTGCGGGCATG
GGAGATGCCGAGTACATATATGCGTTTCAAAAGGTTATACTGCCCATAGC
GACCGAGTTTGCCCCGGAATTGGTTATAATTTCCGCTGGGTTCGATGCTG
CTGCGGGGGATGAGCTGGGTGAGTGCCTTGTGTCCCCCGCGGGGTATGCT
CACATGACTCACATGTTAGCGGGCCTTGCAGGCGGCAGGATGGTGGTGGC
GCTAGAAGGCGGTTATAACCTTGATAGCATTTCCCAGTCCGCCTTGGCAG
TTACTAAGGTGCTTCTAGGAGAGCCGCCGGACGAGCTGCCACCTTTGAAG
GCCAATGAGGAAGGTACCGAAACCGTTTGGTTGGTAGCTAGAGAGCAATC
CAAATATTGGAAGTCAGTGGACCCCAAAGCATGCGAACCTCAGGCTGATG
TTGAGCCTATTAGCTTTAGTGTCCCTGAGATCCTAAAAGCCCACCGTCAA
CATTACTTGTACACCAAACATGATATGATGCAAGTCCCTATGATGACGCC
AGAATTAGAAGAAAAGTTTAGTAGCCAAATCATGTGTACTTCCGACATTT
TCGAATCAAAGACCTTAGTAATCTTTGTTCATGAATTTGGGAACTTGAGG
TTAGAGTTAGAGTCTAGCACAACATGTGATGTACATCTGGAGAGATCCTA
TCTAATAGACTTCTCCAAGGAGCTAGTGGGCTGGGTTAAATCAGAGGGGT
ACAGCTTGTTAGACGCGAATCTGTACCCCAAACCGTCCACAACACCAACG
CCCAATTTAAGGCATAAGACGATGGAGGAAGTCGGAAGAGATGTGCTTGT
CTATCTTTGGGATAACTACGTCCAGCTAAGCGGCGCGGAAAGGGTTATCT
TAATAGGGCACGGCCCTGGGTGTAAGCCCCTTGTGGATTTACTAAATAGA
AGAACAACCTCTGTGACGAAGAGCGCCAAAGCGATTATACAGGTGGTCGG
GTCTCAGCGTATGCCGAGTTACCCGTCTGATGTGGACGATGCTAGGCCCT
GGTACCAGAAATCCTCACTTGTTATCGTGCCTCAGTCTCATCCCGTGATG
GGCCCTCATATTAAACCAAAGGACATTCGTAGGCATGGTGTCATGGTACC
TATCGACGAGACTAGACAGATCAAGTTAATTACACGTGCCCTGCCTGCGA
TTAAGCAGTTTGTACAGGAGACTTTATCAAGTTTTCCACTAGCAAACAGG
ACGAATCGTCCA

Seq. ID No: 57

>DAC_17ATGTCAAAGCGTAAAGTTGCGTACTTTTATGATCCAGATGTCG
GCGCTTACACATATGGTTGGAGTCATCTAATGAAGCCTCATAGAATGCGT
ATCACTCACGAATTAGCAACCGCTTACGGGATGCTAGATAAAATGCATGT
TCTTCGTCCCAAGAGGGCGACCCCCGAAGCTATGACAGCGTTCCACACTG
ATGAATACGTCCAGTTTCTTCATTCAGTCACACCTGAAACGGCTGATAAG
TTGACAGGTCAGAAAACTAGATTTCTGGTTGGTGACGACAATCCGGCTTT
TGAGGGTGTTTTCGAATTTTGTTCCATTTCAGCCGGAGGATCAATTGGAG
CGGCGGAGAGGATAGCTTCTGGCGCTGCTGATATAGCTATCAATTGGGCA
GGGGGTTTGCATCACGCGAAGAAGAGAGAGGCGGCCGGGTTCTGCTATAT
AAACGATATAGTCTTGGGCATCCTAGAGTTATTAAGGACATACCCGAGAG
TTCTATATATCGACATTGATTGCCACCACGGTGATGGGGTCGAAGAAGCC
TTCTACACTACGGACCGTGTCATGACGTGTTCCTTCCATAAGTTTGGGGA
GTTCTTTCCTGGTACCGGCACACAGGAGGACACCGGGACCGGAAAGGGGA
AAGGCTACAGCGTCAATGTGCCGCTTAAAGATGGCATTCAAGATGAGTCA
TTTAAGTCCGTGTTTGACCCAGTGATCTCTAAGATCCTAGAAGTATTCCA
GCCATCTGCCGTTGTGTTACAATGCGGAGCCGACAGTCTTGCCGGCGACA
AGTTGGGGTGTCTGAACCTTACCATGCAAGGTCATGCTCATTGCGTCCAG
TTTTTGAGAAAATCTAACATTCCATTAATCCTACTAGGTGGTGGAGGATA
TACGGTTAAGAATGTAGCAAGGGCTTGGACATATGAAACCGCATGCGCAA
TCGGAATAGAAAACGAAATAGACTTGAACATGCCGTGGTCTCAATACTTC
GAGTGGTTCGGCCCGACCTACCGTCTGGAAGTTCCAGAAAACAACATGGA
AGATATGAACGTTAAAGAAGGCACTTTGGATCATGTTAGAACGACTGCAC
TAGCCCAGCTGCAGCAGCTTGCGTCACGTTGTGCACCTTCTGTGCAAATG
CAGGACGTCCCGCGTACATCCTTAGGCGGCCACTTGGGCTTCAAACGTGA
CAAGAGAGAGCATCGTGATGAACTGGACGAGAGATTGGCCCAGCATACTC
GTTATTTGTACGACCTTCAAGAGTCCGAGTCAGAAAGCGAGGATACGGAA
AGCTCAGACAGTGATGCTTCCTCTGTGTCTTTCGTAAATAACTGGAGAAG
AGCGCCCCATAGAGCTAACAGCCTTCCCCGTATCTTGTCTGGTAGGCATT
CCAGCAATCCCCCTGGACACATAAGTGCCTCCGAAAGAAGAAGAATGAGC
ATCGTAACCGGCAAGTACTTCGACATCCCCATACACGAGAGCGGTTATAA
CCACTACGAATATGGCGCAGCCCCCACTAAATCATCAAAAAGGATCTTTT
TCCAATCAGGTCTTGACATTTACAACGATGACAATGATTTCGAGGGCATT
ATCAATGCTAGAACCTCAGTTAGCAACGGATTTGGTAACGGAATACACGA
TCTTCACGGCTTAATGGAACGTGGAGGCAGAAGTTTGAACGAAAACCTGG
AAGACGGGGACGATGAGGTGGAAGGTGAGGAATACGAGGATGACGCAGCC
ATGTCAGATAGT

Seq. ID No: 58

>DAC_18ATGGAAGAACACTTCTGGGATGTCCTATACAAGGACAAATACT
CAAAGCTGTTGAGCAAGGCTCGTGATTTCTTGGACGATACCGGCGGACCG
GGAGACGATGTCTTAGTCTTTATCTCTTGTGGGATGGACGCGTGTGAACA
TGAATATGAAAGTATGTCAAGACATAACAGGAAGGTCCCTGCGAGTTTCT
ACCATCGTTTCGCAAGAGACGCATGTGCGTTCTCTGACCGTTACGCGGGA
GGGAGACTAATTTCTGTCCTTGAAGGGGGATACAGTGATAGGGCATTGAT
CTCTGGAGCAATGGCACATCTAAGCGGATTGGTAGATACACCTGATGGTA
TTCAGGTCGATGAGCAGTGGTGGAACATACCGAACCTTGTGAAATTGGAA
GCTGCCACCAAAAAGCGTAGAGGTGGACGTCCTAGCCTGCCTGCGAAAGG
CTCAGTGGAGCCGTGGATAGAAAGGACGTTATCCATCTTTTCCAGCATAG
ATGGTAGTGCGAGCACAACCTCATCAAGGAGTACGTTTATACCACCGTCA
TCTAGGACATTAAGAGATCGTACAAAGGGGAGGGAGGCTATGCCTAAATC
CCCGCCAGCTTCCAGTGCTTCAACCAAGCCTGTGTCCAGATCAAAAGTAA
AGCCGGGTGCAAATATCAAGAGCGGAGATGAATCTTTCGCTTCAACAGGA
AGTAGTCCGCTTACCTCCCCTTCTCCCAGTAGCTCTGAAGACGAAGCACC
GCCCATTAAACGTTTG

Seq. ID No: 59

>DMAT_1ATGACCATAATAAATTCCCGTATAATTGACATTAGACAGAGTA
CCTTCGAAGAGAGTATCCCCGACCAAGTAACGGCCGGTTTATCTACCACC
CCCAAGACCCTGCCCGCACTGCTGTTTTACTCAGGCGAGGGTATTCGTCA
TTGGATTGAGCATAGTACCGCAGCCGATTTCTACCCGAGACACGAGGAGC
TGAGGATATTACGTGCGAGGGCTGCGGAAATGGTCGATAGTATAGCCAAC
AATAGCGTGGTAGTCGATTTGGGTTCTGCTTCCTTAGATAAAGTGCTTCC
ACTACTTGAGGCCTTAGAAGCCAGTAAAAAGAATATTACCTTCTATGCTT
TGGATCTTAGTTTCAGTGAATTACAAAGTACCCTGCAGAGCTTGCCATAC
GAACAATTCAAGTTTGTGAAGATAGGGGCGCTACACGGGACCTTCGAAGA
TGGTGTTCAATGGTTAAAGGATACACCTGGCGTACAGGACAGGCCTCATT
GCCTATTATTATTTGGGTTGACTGTTGGGAACTACAGTAGACCAAATGCC
GCGAAGTTTTTGCAAAACATTGCCTCCAACGCACTGGCGGCGTCACCAGT
CCAATCTTCAATTCTGTTGAGCCTGGACTCATGTAAGATGCCAACCAAGG
TGTTGCGTGCATATACAGCCGAAGGGGTAGTTCCGTTTGCATTGGCCAGT
TTAGACTACGGGAATACGCTTTTTGCTCCTAACAAAATGGGGGAGAAGGT
CTTCCAGCCTAGTGATTGGTATTTTCTGTCTGAGTGGAACTACATGCTTG
GCAGGCATGAGGCCTCCTTGATCACAAAAGGGAAAGAGGTGCGTCTTGGT
GGGCCCTTGAATGATATCGTGATAGAGAAACACGAGAAAATTCGTTTTGG
ATGTTCCTATAAATACGATACCGATGAAAGACAAGTTTTATTCGGATCTG
CAGGTCTGACTGACGTAAAAGAATGGTCTGTCGAAGGTTGTGACGTGAGC
TTCTATCAACTACAGATGTGTCCTAAC

Seq. ID No: 60

>DMAT_2ATGACCATATCCGCCCCACCAATCATAGATATTAGACAAGCGG
GCTTAGAGTCATCCATACCAGATCAGGTGGTTGAGGGATTAACAAAAGAA
GTAAAAACCCTGCCAGCACTGTTGTTTTACTCCACAAAAGGTATACAACA
CTGGAACCGTCACAGCCATGCGGCTGACTTTTATCCGAGGCACGAAGAAC
TGTGTATCCTAAAAGCAGAGGCCTCAAAAATGGCCGCGAGTATCGCCCAA
GACTCTTTAGTTATAGACATGGGCAGTGCAAGCATGGATAAAGTGATTCT
GTTACTTGAGGCGCTTGAGGAACAAAAGAAGTCAATTACATATTGCGCGC
TTGACCTTTCTTATTCAGAGCTGGCCTCCAATTTCCAAGCTATCCCAGTA
GACAGATTTCACTATGTTAGGTTTGCTGCTTTGCACGGCACATTTGATGA
CGGACTTCATTGGCTGCAAAACGCGCCAGATATCAGAAATAGGCCCAGAT
GCATACTGCTATTTGGTCTGACAATCGGTAATTTCTCCAGAGATAACGCC
GCAAGTTTCTTGAGGAATATTGCTCAGTCTGCGCTTTCTACGAGTCCTAC
ACAGTCGTCAATCATAGTCAGTCTGGATAGCTGCAAACTACCAACTAAGA
TACTTCGTGCATATACGGCAGACGGTGTTGTGCCATTTGCGTTGGCCTCT
TTGTCATATGCCAACAGTTTATTTCATCCCAAAGGAGATAGGAAAATCTT
CAACGAAGAGGATTGGTACTTTCACAGTGAATGGAACCATGCTCTTGGTA
GACATGAGGCGTCTCTGATCACACAGAGCAAAGATATTCAACTTGGGGCA
CCTTTAGAGACGGTTATTGTCCGTAGAGACGAAAAGATCCGTTTTGGATG
TAGCTATAAATACGACAAGGCAGAAAGAGATCAGTTGTTTCACAGTGCAG
GACTGGAAGATGCAGCGGTCTGGACTGCTCCGGACTGTGATGTTGCGTTC
TACCAACTAAGGCTAAGGCTAAAT

Seq. ID No: 61

>DMAT_3ATGTCTAAACCGAATGTACTTGATATCAGACTGGCCACGTTTG
AAGACTCTATCGTGGATCTGGTGATTAACGGTCTGCGTAAGCAACCTAAG
ACCCTTCCGGCATTACTATTCTACGCAAATGAAGGGCTGAAGCACTGGAA
TCACCATAGTCATCAACCCGAATTTTACCCCCGTCATCAGGAGGTACAAA
TATTGAAAAAGAAGGCCCAAGAAATGGCAGCTTCCATTCCAATGAATAGT
GTCGTTGTGGACCTAGGATCTGCCTCACTAGACAAGGTCATCCATCTTTT
AGAGGCGCTAGAAGTACAGAAGAAGAATATCTCATACTATGCCCTGGATG
TATCAGCGAGTCAGCTGGAGTCTACACTAGCTGCAATACCGACCCAGAAC
TTCCGTCACGTGAGATTCGCGGGGTTACACGGAACCTTCGATGACGGGCT
TCACTGGCTAAAGGAAGCACCGGAGGCTAGAGACGTGCCGCACACAGTAT
TGCTATTCGGTCTAACAATAGGGAACTTCTCCAGGCCTAACGCAGCAGCA
TTCTTATCAAATATCGGTCAACATGCATTTCAGGGTAAGTCAGGCGATCA
ATGCAGTATTCTGATGTCTCTGGACAGCTGCAAAGTCCCAACACAGGTCC
TAAGAGCCTATACTTGTGAAGGCGTTGTTCCTTTCGCCTTGCAAAGTCTA
ACTTATGCAAATGGCCTATTTTCAGAGAAAAATAAAACCCAAGCCAGCGG
CGATGTTCAACACAAGGTCTTCAACCTGGATGAGTGGTATTACCTATCAG
AATGGAATTTTGTTCTGGGCAGACACGAGGCTTCTTTAATACCGCGTTCT
AAGGACATTAAGCTATTACCGCCGCTTGACGGAATATTAGTCTCAAAGGA
CGAAAAAGTCAGGTTTGGATGTAGTTATAAATATGACCAAGAAGAACGTA
TGGAACTGTTTGCCGCCGCCGGAGTCAAGAATGAAGTAACATGGAGCGAT
GAAGGTTGCGATGTAGCATTCTACCAATTAAAGCTATCA

Seq. ID No: 62

>DMAT_4ATGGGTTCCATAAATCCGCCACAAATTCTGGATATCAGGAGAT
CAAAATTTGAGGAATCTATTCCGAAGCAAGTGGAGGCCGGGCTGCTAAGT
AGTCCTAAGACTCTTCCAGCATTACTATTTTACAGCACGGAAGGAATACA
GCATTGGAATCGTTATAGTCATGCTTCAGATTTCTACCCTAGACACGAGG
AGATTCAGATACTGAAAGATAAGGCAACGGACATGGCGGCTAGTATAGCT
GACGGATCAGTCGTGGTAGACCTTGGTTCCGCCAGTCTTGATAAAGTTAT
TCATCTACTGGAGGCGCTGGAAGCAGCCCAGAAGAAGGTTACTTACTATG
CCCTAGACTTATCATTCTCCGAGCTGACGAGCACCTTGCAGGCCATACCT
ACCGATCAATTCGTTCATGTCCAATTTTCAGCACTTCATGGGACATTTGA
CGATGGCTTGCAGTGGTTGAAGGAGACTCTGGTTATAAGAGACCAGCCCC
ATTGCTTGTTATTATTTGGACTTACTATTGGCAATTTTAGCAGACCCAAC
GCCGCGAAGTTTTTACACAACATAGCGTCTCATGCTCTAGTGGAGAGTCC
AAGTCAATCAAGTATTCTTTTAACCCTGGACTCCTGCAAGGTCCCGACAA
AAGTAATCCGTGCATACACGGCTGAGGGCGTCGTCCCTTTCGCATTGGAG
TCATTGAAGTATGGGAATACTCTATTCCAACAGGATGCCGGAGAGAATGT
GTTCGATCCCGAGGACTGGTATTTTCTGAGTGAATGGAATTATGTCTTAG
GAAGACATGAAGCATCATTGGTACCTAGGTCCAAGGACATTAAACTTGGT
AGGCCACTGGATAAGATAGTTGTAGGGAAGCACGAGAAGGTCAGATTCGG
TTGTTCTTATAAATACGATTCAGAAGAGAGAAAAGAACTATTCGGAACAG
CCGGGTTAAGGGATGTAAAGTCTTGGTCAAAAGAAGGGTGCGATGTCGCA
TTCTATCAATTAAAATGCTGCCCCAAT

Seq. ID No: 63

>DMAT_5ATGCCGGCACTGCCTGTCATAGATATCAGATCCAATCACGTTG
AGGACTCCCTACCCGAACAAATAATAAAAGGCCTTACCTCACAACCAAAG
ACTCTGCCGCCGCTACTTTTCTATTCAAATGAGGGCCTTGAACATTGGAA
TCATCATAGTCGTCAGCCCGACTTCTATCCAAGAAGGCAGGAAATCGAGA
TATTAAAACAAGGAGGAAACGATATTGCAAGATCCATTGCGCCCTCAAGT
GTCATTCTGGATCTGGGATCAGCAAACCTGGAGAAGGTGGGCTATCTGCT
GGAAGCTCTGGAGGCGCAGGAGAAAGACGTCCTATATTTCGCCTTAGACA
TCTCAGCGCCGCAACTAGCGACTACGCTAAAAGAAATACCAAGTAGCAAC
TTTCGTCACGTCCGTTTTGCCGGATTGCATGGTACGTTTGAGGATGGTCT
GAGATGGATAAATGAGACCCCTGAGATTAGGGATTTACCGCACTGCGTAC
TTCTGCTGGGGCTTACGATCGGAAATTTTTCAAGGCAAAACGCCGCTGCT
TTCTTGCAAAACATAGCAAATCATGCCCTTACTGGTGCTTCAAAGAACAA
ATCAAGCATATTATTAAGCCTAGATAGTTGCAAAGTACCCACAAAGGTCA
CTAGAGCATATACGTCAGACGGGGTAGTTCCATTCGCTCTACAGGCGTTG
ACGTATGCCAAGGCGTTATTGTGCGATAGGATCGACAATGGGATAGATGA
GAAGGTTTTATCATGTAACTTGAGGCCCGAACATTGGCACTATCTTTCCG
AGTGGAACTTTGCCTTAGGGAGGCATGAAGCCAGCTTAATTCCAAGGTTC
GGAGATGTGTGCCTGGGCTCCATGTTACAAGATATCATTGTAAAGAAAGA
AGAGAAAGTCCGTTTTGCCTGTAGCTACAAGTATGATGCTAAGGAGAGGC
AAAAGCTGTTCCTGGACTCTGGAGTAGATCAAGGGATGGTCTGGACAAAC
GAAGGATGCGATGTGGCAATCTACGAGTTGAAGTTAGCA

Seq. ID No: 64

>DMAT_6ATGCTTTACAAGCCCAAAGTTTTGGACATAAGGTCAGGATCAG
TAGAAGATAGCCTACGTCATTCCGTGATGGACGGTATTCGTGAAGACCCT
AGAACCTTGCCTACCTTAATTCTATACGGTCCGGAGGGTTTACAACATTG
GGACGATCACTCACATGCTCCCGACTACTACTTACGTCACGAAGAGTTGC
ATATACTAAGAAGCAGAGCTTATGAAATGGCGGAGACTATCGCGGATAAT
ACTGCAATGGTTGATCTGGGCTCCGCGCAGGTAAGTAGATTCCACGAGTC
ACCGTGCCTACTGGCGCCGACTTTATCTCTCGACAAAGCAGCTCTTCTGC
TAGACGCCCTTGAGGTTCAGGCTAAGAATGTCACATATTATGCGTTGGAC
CTTGACCATGCGGAATTACAGAAAACGCTGTGTAGACTACCTTTAGGGAA
GTACAAACATGTTCAATGCGTTGGCTTACAGGGAACCTTCGAGGACGGAC
TAGAGTGGATAAAGAACGATCCGGAGCAGTCTAGGCGTCCGCACTGTCTA
CTTTTCCTGGGTTCAACTATTGGTAATTTCTCAAGAGAAAATGCTGCCCG
TTTTATAAGATCAATGGCAAGTTCTGCCTTTCTAAGTGAGTCAGCTAAAT
CTTCTATTATCTTGTCTATAGATTCCTGCAAATTGCCCACTAAGGTTCTT
AGAGCGTATAACTCAGAAGGTGTTGTTCCCTTCGCGATGGCCGGCTTGAA
GCATGCGAGCGCGATCTTGTGTGAAGCCGCGTGTAGGCAAGAGGACGCAG
TTACCGAAACTTTTCTGCCGGACGACTGGTATTACTTATCACATTACAAC
CACGTATTGGGTCGTCATGAGGCCAGCTTCACACCAAGGAACAGAGATAT
TCAATTGGGTTCTCCACTAGAGGACGTCGTAATAAGACTTGGAGAGACAA
TCCGTTTCGGCTATTCACATAAGTATGATTTTGCCGAGATTGAGCAACTT
TTTCGTGAGGCCGGGGTCGCTGCTGTTAATAGTTGGGGAGCGGTTGGCTG
CGACCTTTCATTCTATCAGCTGGGAACGGCG

Seq. ID No: 65

>DMAT_7ATGGCAGCACCTTCAGTTATTGACATCCGTTCTCACCTGGTGG
AAGATAGTTTACCAGATCAGGTGGTGAAAGGGTTAGGGTCCGATCCCAAG
ACCCTTCCAGCTTTGTTGTTCTATTCAAACGAGGGCTTGGAGTACTGGAA
TCATCACGCACGTCAACCCGATTTTTATCCAAGGCACCAAGAAATTGAAA
TTTTAAAGCGTAAGGGTGATGAGATCGCCAGGAGCGTTGCGCCTAATTCC
GTCATTCTGGATTTGGGATCTGCTAACCTTGAAAAAGTCACTTATCTACT
AGAGGCACTGGAAGCGCAAGCAAAGAACGTAACTTATTTTGCCCTTGATT
TATCAGCTCCTCAACTGATGAGTACCCTGAAGGCGATTCCCACCACTAAA
TTTCGTCATGTCAGATTCGCAGGATTACATGGCACGTTCGTCGATGGATT
AAGATGGATATCAGAGACTCCTGATATACGTGACCTACCTCATTGTGTTC
TTCTGTTCGGCCTTACTATTGGGAACTTCAGTAGGCCGAACGCAGCCACC
TTTCTAAGAAACATTGCGAGTCAGGCACTTCGAGGGGCGAGCGAGGACAA
GTCTTCCATTTTCCTATCATTGGATTCCTGCAAAGTGCCAACCCAAATCC
TGAGGGCCTACACATCCGATGGTGTGGTTCCATTCGCTCTTCAGAGTCTG
GCTTACGCTAAAACTCTATTCTGTGAACAGACGCAAAACGATTTTAATGA
GAAACCTTCAAGTTGTCATTTAAACCCCGATGACTGGCATTACCATAGTG
AATGGAACTTTGTACTGGGGAGACACGAAGCCTCTCTTATTCCGAGACTA
AATGACATACATTTGGGGCCTCTTCTACATGATATCGTAGTGAAAAAGGA
CGAGAAAGTCCGTTTTGGATGCTCTTATAAATACGATGATCTGGAAAGGG
ATAAATTGTTCGTTGATGCTGGAGTAAAAGACGAGATGGCTTGGACCAAT
GAAGGATGCGACATCGCTATTTACGAACTGAAGTCCATG

Seq. ID No: 66

>FEX1ATGATCTTCAACCCAGTGATATCAAATCACAAGCTGTCACACTAT
ATACACGTCTTCTGCACGTTCACAACCTTCTGCATTTTGGGGACGGAAAC
AAGGCAAGCCATAACCGCGCTAAGTACATATACTCCAGCTTTCGTTACTG
CTCCGACAGTGCTATGGAGTAATTGCTCTAGTTGTATGTTAATGGGCATA
ATGCAGTCATTGAACGCGTACACTTGGATGAAGGACCATCAAGTTCTTTT
CCTGGGTGTCACTACCGGTTATTGTGGGGCCTTGTCATCTTTTTCCTCCA
TGCTGTTAGAGATGTTCGAGCATAGCACCAACCTGACAAATGGGAACATT
GCGAATCATACTAAGTTACCTAATCGTGCCTATGGGATAATGGAATTTTT
ATCTGTCCTGCTGGTGCATTTAATGGTTTCTATGGGAAGCCTGATCTTTG
GTCGTCAGTTGGGAAAGGAAGTAATCGTCGCTTACGGCTCATCCAGTTTT
TCTAAACCTTACACACCACCGTCTGACACAGTAAAAGAGAACGCCGGAGA
TGTAGATACACAAGAGATGGAGAAGAATATATTAGAGTTTAAGTTCAAAA
CGCCGGCGCCCTTCTTCAAGAAGTTCTTCGACATAGTCGATAAACTAGCT
TATGCCCTAGCTTTTCCACTGATCATATTGTTCGTAGTCCTATGTGCGTA
TTACGAAAACTACTCCCGTGGAAAGTGGACACTACCGTGCTTGTTCGGCA
TATTTGCTGGTTTTCTTCGTTACTGGTTAGCAGAAATGTTTAATAAAACC
AATAAGAAATTCCCTCTGGGGACTTTCCTGGCCAATGTTTTTGCGACCTT
GCTGATTGGTATCTTCACGATGGTGCAAAGAGGAAAGAAGCACTTTTCCA
CTGACGTACCCATCGTTAATTCCTTGAACAGCTGTCACATAGTGAGTGCA
TTGATAAGTGGGTTCTGTGGGACTCTGTCTACGATATCTACTTTCATTAA
CGAGGGCTATAAATTGAGTTTCATCAACATGCTGATTTATTACACTGTAA
GTATAGCAATATCCTATTGTCTTCTAGTTATTACCTTGGGGTCATACGCG
TGGACAAGAGGATTAACTAACCCCATTTGC

Seq. ID No: 67

>IDI1_for_fusionGGCGGGTCCGGTGGTTCAAGTGGCAGCGGTGGCT
CCTCATCAACCGCGGACAACAACAGTATGCCGCATGGCGCCGTCTCAAGT
TATGCGAAACTGGTACAAAATCAGACACCGGAGGATATCCTGGAGGAATT
TCCCGAAATCATCCCTCTGCAGCAGAGACCTAACACCAGATCCAGTGAAA
CGTCCAACGACGAGTCTGGTGAAACGTGTTTCTCTGGTCACGATGAAGAG
CAAATCAAACTTATGAACGAGAACTGCATAGTGCTAGATTGGGATGATAA
TGCTATAGGGGCCGGGACCAAGAAGGTGTGTCATCTTATGGAAAACATCG
AGAAGGGCTTATTACACCGTGCGTTTAGTGTATTTATATTCAACGAGCAG
GGCGAGTTGTTATTACAACAAAGGGCTACGGAGAAGATCACGTTTCCAGA
CTTGTGGACCAACACTTGTTGTAGCCATCCACTGTGCATTGATGATGAAC
TTGGTCTCAAGGGAAAGCTAGATGACAAAATCAAAGGTGCTATCACCGCT
GCCGTGAGAAAGTTGGACCACGAGTTAGGGATTCCTGAGGACGAGACAAA
AACTCGTGGAAAATTTCATTTTCTAAATCGTATTCATTTCATGGCACCTT
CCAACGAACCGTGGGGCGAGCATGAAATTGACTATATCCTGTTCTATAAG
ATAAATGCGAAAGAAAATTTAACTGTCAATCCGAATGTCAATGAGGTGAG
AGATTTTAAGTGGGTGTCCCCTAATGACTTGAAGACGATGTTTGCTGATC
CTTCATATAAATTTACCCCTTGCTTCAAAATAATATGCGAGAACTATTTA
TTCAACTGGTGGGAACAGCTAGACGACTTGTCTGAGGTCGAGAATGACAG
GCAAATTCACAGGATGCTG

Seq. ID No: 68

>INMT_1ATGGCTGCTCCTCACACCTCTCAACAAGATTATATCGACAACT
TTAACGCCAGAGACTATCTGCAAACAAGCTACACCCCGGGGAAAGGTATT
CTTTTTGGTGAGTGGATTGAATTTGCTACGCAGAATCTTCACGAAACCTT
TACGACAGGTGGTGTAAGAGGTGATACGTTGCTTGATTTCGGAACTGGGC
CAACAATCTACCAGTTAATATCAGCCTGCGAAGTCTTCGACAAAATCATC
GTAAGCGACTTTCTGGAACAAAATAGGGCGGAGTTCAGGAAATGGCTAAA
TAAGGACCCTGACGCCTTCGACTGGACACCCATAATCAAAGGAGTGTGCG
AACTGGAAGGTAATAGGGAAGACTGGGAGAAGAAAGCCACAAAACTGCGT
TCCAAGGTTAAAGAGGTTCTAAAATGCGATGCCCTAAAAAGAAACCCTTA
CGACCCCATTGTCGTGCCACCAGTAGATTGTTTGTTAAGCTGTTTATGCT
TGGAGGCACCCTGTAAAGACATTAAAAGTTACTGCGAAGTCCTGAAGAAT
TTTCAAAGTCTGATAAAGCCCGGTGGGCACCTTTTAATCCTTTCAGGTCT
GAATGCCACATTCTATTATGTAGGTAAAACTTATTTCTCAAGCATGACAA
CAAAGAAGGAGGAGCTAGAAATGGCCTTCAAGGAAGCGGGATACATCATA
AAGAAGGCAGTCTATACGCCGAGAGCGGATAAATCCAAAATAGACGTTGC
CGATTACGAGGGCCATTACTTTATCCATGCCCACAAGCCAAAG

Seq. ID No: 69

>INMT_2ATGGCTGCACCCCATACCTCTCAACAAGACTATATTGATAATT
TCAATGCGCGTGACTACCTGCAAACAAGCTACACCCCAGGAAAAGGGATC
TTATTCGGTGAATGGATAGAGTTCGCGACGCAAAACCTGCATGAAACTTT
TACCACCGGCGGGGTTAGAGGCGATACCCTTCTGGATTTTGGGACGGGAC
CGACCATATATCAGTTAATATCTGCGTGCGAGGTCTTCGATAAGATAATA
GTGAGTGACTTATTGGAACAGAATAGAACCGAGTTCCAAAAATGGCTTAA
TAAAGACCCTGACGCATTTGATTGGACTCCTATTATTAAGGGAGTTTGTG
AGTTAGAAGGGAATCGTGAAAATTCTGAAAAGAAGGCTGAGAAACTGCGT
TCAAAAGTGAAACAGGTACTAAAGTGCGACGCTCTAAAAAGGAATCCCTA
CGATCCAGTGATCGTTCCACCTGCAGACTGTCTATTAGTCTGTTTGTGTT
TAGAAATTCCTTGTAAGGACATGAAATCCTACTGTAACGTGCTGAAAAAT
TTCAAAGATCTGTTGAAACCAGAAGGCCAAATACTGATATTAGGAACTTT
AAACGGAACTTATTACCACGCAGGAAAGAAGAGATTTAGCCTTTTATCTA
GCAAGAAAGAGGATTTAGAGATGGCTTTTAAAGAAGCCGGCTATATAATT
GAAAAGGCCGTCTATACGCTACGTGCCGATAAGTCTAACATCGACGTTGC
CGACTATGAAGGTCATTACTTTATACATGCTCATAAGCCCAAA

Seq. ID No: 70

>INMT_3ATGTCTGATTTTACGAACACCAGAGAGTACGAGGAGCAGTTTG
ATCCGCGTTTGTACCTGGAGACGTACTTTCACCTTGGTTCAGGGTCTTTA
GCCGACGATTTCTTACGTTTCGTTCTGGACAATTTCAACAAAACATTCAA
GTCCGGGGCTGTCAAGGGAAGCACATTAATTGACATTGGGACTGCCCCCT
CAATTTATCAGTTATTATCTGCGTGCGAGTCTTTTGATGACATAATTGTG
ACTTGGCACACGAACCGTGAACTAAAGGAGCTACAAAAGTGGCTAAACAG
CGAGGCAGATGCGTTCGATTGGTCATCAATTGTTAAGCATGTCTGCGAAA
TTGAGGGAAACAGGATGGCTCAGAAAGAGAAAGAAGAGAAACTAAAGGGT
AAAATTAAACAGGTTCTAATGTGCGACGTATCCAAAAGTAACCCCTTGAG
CCCGCATGAAGTCCCTAAAGCTGATTGCCTGCTGACAACCGTCTGCCTAG
AGGCCGCTTGTAAAAATTATGAAAGTTACGGCACCGCGCTTAAAAACCTA
TCCAATTTGTTAAAGCCAAAGGGTCACCTACTTATGGCAGGTGATCTTGG
AGCGAACTATTACGAGGTAGGAAGTAACAAAGTATTCTCCCTGCCCGTCA
ATGAAAAATTCTTAAAGAAGGTCATTAGTGAGTCCGGCTATGAGATAATC
CAACTTGTCTCATTCGGGAAGCCCGAGAATGCCGATTTTGAAACCAGCGA
CTATGAGGGTTTTTATTTTGTCCATGCCCAGAAAGTG

Seq. ID No: 71

>INMT_4ATGGACTGCTTGATTTCTTGCCTATGTTTAGAAGCCCCCTGTA
AAGATTTGGAAGATTTCACGAATACGTTAAAGAAGTTTAAGGAATTGTTA
AAACCCGGAGGACACATAATTATACAGTCAGTGCTGAATTGCTCCCTTTA
CTTCGTCGGGAACAAATCATTTTCATGCCTTAGCATAACTAAGGATGAAC
TGGAGCAGGCATTCAAGGAGGCCGGGTATGAAATTGTAAAGTTAAAGGTT
GTCCCACGTTCTGAGAAAATTTGGGCTAACGTGTCCGATCACTCCGAGTA
CTATTATATACATGCTCGTAAACCACAG

Seq. ID No: 72

>INMT_5ATGAGCGACTTCACAGGAAAGAATGAATATCAGACCTTCTTCA
ACCCTAAGGCTTACTTAGAGAGCTACTATCAGTTAGGGTCCGGCAGTATG
GGTGATGAGTATCTTCAATTTGTTCTAAAGGAGCTGGCAGAGACGTTCAA
TCCAGGAAAGGTCAAGGGGGACACTCTAATTGACATTGGAACCGGACCTA
CTATATATCAATTACTGTCTGCGTGTGAGGCGTTTAAAAATATAATTGTC
TCCGATTTTACTGATAAAAATCGTGAAGAATTTAATGTGTGGCTGAAAAA
TCAACCTGGAGCGTTTGATTGGAGCCCAGTTGTTAAACATGTTTGCCGTC
TGGAGGGAGATAGAATCCCTTGGGAACAAAAGGAAGAAAGACTTCGTAAA
ACGATCAAACAGGTACTAAAATGCGATGTGTTTAATATAAATCCAATTGA
CCCGGTTACCATTCCACAAGTTGATTGCCTGCTATCATGTCTGTGTTTGG
AAGGTGCTTGCAAAGATTTTGAATCTTATATTACTGCCCTTAAAAATATG
ACGACACTATTAAAAATCGGCGGTTATCTGGTAATGACCGGGGACCTTGG
AAATACGTATTATATGGTAGGGGATGTGAAGTTTAGCGGGCTGAACTTAA
ATGAGAACTTTCTTAGAGAAGCAATAACTGGAGCGGGCTACGTTATAGAG
TCATTCCAACAATCTAAAAAGACCGAAGATAGTGTAGAAGATAAGGCGGA
TTTTACAGCTTACTACGTGATAGTAGCGAGAAAAGAGAGGAATGTG

Seq. ID No: 73

>INMT_6ATGGAGTCAGGGTTTACGAGTAAAGATACATACTTGAGTCATT
TCAACCCGAGAGACTACCTTGAAAAATACTACAAGTTCGGTTCAAGACAC
AGCGCGGAATCCCAGATTCTGAAGCACCTATTGAAGAATTTGTTTAAAAT
ATTTTGCCTAGACGGCGTAAAAGGCGATTTATTAATTGATATAGGGTCTG
GCCCCACAATTTACCAACTACTGTCTGCGTGTGAATCATTCAAGGAAATA
GTCGTGACAGACTATTCAGATCAAAACTTACAGGAGCTTGAGAAGTGGCT
GAAGAAAGAGCCAGAGGCTTTTGACTGGAGTCCAGTAGTCACCTATGTTT
GCGACCTTGAGGGCAACAGGGTTAAAGGGCCGGAGAAAGAAGAAAAATTA
AGGCAGGCCGTAAAGCAGGTATTGAAATGCGATGTGACCCAGTCCCAACC
TCTGGGCGCAGTACCTCTGCCGCCCGCTGACTGTGTCTTGTCTACCCTGT
GTCTGGACGCGGCATGTCCTGACCTACCTACTTACTGTAGGGCATTAAGG
AATCTAGGCTCTCTATTGAAGCCGGGTGGCTTTCTAGTAATCATGGACGC
TTTAAAGAGTTCCTACTACATGATTGGTGAGCAGAAGTTTAGTTCACTGC
CGTTGGGCCGTGAGGCGGTGGAGGCGGCAGTAAAAGAAGCGGGCTACACG
ATTGAATGGTTCGAAGTTATCTCCCAGTCATATTCTAGCACGATGGCGAA
CAACGAGGGGTTGTTTTCATTGGTGGCCAGGAAACTGAGCAGACCTTTG

Seq. ID No: 74

>INMT_7ATGAAGGGTGGTTTCACAGGTGGAGATGAGTATCAAAAGCACT
TTTTGCCCAGGGACTATTTGGCTACGTATTACTCCTTTGACGGGTCCCCT
TCCCCGGAGGCTGAAATGCTGAAATTTAACCTAGAGTGCCTACACAAAAC
TTTTGGTCCTGGAGGGCTTCAGGGTGACACTTTAATAGATATAGGAAGCG
GACCCACCATTTACCAAGTGCTTGCAGCATGTGACTCATTTCAAGATATT
ACTTTAAGCGATTTTACCGATAGAAACAGAGAAGAGCTTGAAAAATGGTT
GAAAAAGGAACCTGGAGCATATGACTGGACACCAGCGGTGAAGTTCGCTT
GTGAATTAGAAGGCAATAGCGGACGTTGGGAGGAAAAAGAGGAAAAACTA
AGAGCTGCCGTTAAGCGTGTGCTAAAGTGCGATGTGCATTTGGGAAACCC
CTTAGCACCAGCCGTATTGCCGCTTGCAGACTGTGTGTTAACCCTATTGG
CAATGGAGTGCGCTTGCTGTTCACTGGACGCATACAGGGCCGCGCTATGC
AACTTAGCGTCTCTGCTAAAACCCGGAGGTCATCTTGTAACAACAGTGAC
CCTGAGACTGCCGTCCTATATGGTAGGCAAGCGTGAATTTAGTTGCGTGG
CTCTGGAAAAGGGTGAAGTCGAGCAAGCCGTATTGGACGCAGGATTCGAC
ATCGAGCAGCTACTGCATAGCCCGCAATCTTACTCAGTCACAAATGCAGC
GAATAACGGAGTTTGCTGTATCGTCGCACGTAAGAAGCCAGGACCT

Seq. ID No: 75

>INMT_8ATGAGCGATATAGATGACGGAGCCTTAGCTTCCGCCCAAGCAA
TCGTCGATGGCAACCGTTTAGCGGGGCAAATTGAGCTTCGTCAGCAGCCT
GACCCTGACAGAGTTTTCGCGGGTGTTCTTAGACAGGGCGAGGCGGTTGC
TTTTTGTGTATGTAACCCGCCGTTTCATGAATCACTAGAACACGCCAGGC
GTGCCGCTGGTGCTAAGTGGCAACGTCTAGGTAGAGCGGTTCAAGGAAAG
GAGATGAACTATCAAGGGTCCCCGGCGGAACTATGTTGTAACGGTGGAGA
AGTGGGTTTCGTGACGAGGATGGCTGAAGAAAGTGCCCAACCGCGTCGTC
AGAGGGCTTGTGTCTGGTTCAGTGCTATGCTTTCACGTGAAAGTTCTATC
GCCCCTGTCCGTGAGCGTTTGGGTGAGCTGGGCGCGAGAAGGAGAGCCTG
GGAGTTACGTCAAGGGAGAACAACGAAGTGGGTAGTTGCTTGGACCTTCT
ACCCGAGGGGAGAACGTGACCAACGTCTACGTGAAATGGCGCAGAGGCGT
GCAGATCCTGAAGCACGTGCTGAAGCGGGTGCAGAAGCTGCGACCGCGAG
AGATGTAGGCGCAGGAGGGGACGGGGCCGATGGCGTGGGTGGGAGTTTAG
TCCGTCGTTCAGCAGGCGCTGGAGGATCAGCCGCA

Seq. ID No: 76

>INMT_9ATGGATTTCACGGGAGGCGAAATCTATCAATCCAGTTTCGACC
CGAAAGCCTATCTTGCTTCCTTCTGCTCACTTGGCTCCGGCAGGGACGAC
ATACTAATGTTCAGACTTAAAAAGTGCTTTGAGACCTTTGGACCCGGTGG
GTTGCGTGGTGACGTTTTAGTTGACATAGGAACTGGTCCAGCCATATACC
ACTTGTTGAGTGCTTGCGAGTCCTTCCCATACATCATTGCTACTGATTTT
ACCGATAACAATCGTCAAGAACTGGAAAAGTGGTTGAGGCGTGAACCTGG
CACGTTTGATTGGCTGGAAACAGTAAAAATTGTCTGTGACTTAGAAGGGG
ACTCCAGGGACGATTGGGTGGAGAAGGAGGACAAACTAAGGTCAAGGATA
CAAAAAGTGTTAAAGTGCGACGTTACGAAGACAAACCCCCTAGATCCAAC
TGTAATTCCACCTGCAGACTGTCTAATCACGGCATTGTGCCTTGAGACTG
CGTGTACGGATATTGATACCTATTTCTGTTCTCTAAGAAATATAACCACT
CTACTGAAACCTGGTGGGCATCTTGTTTTGATCGGAGTTCTGGGTAACAG
TTTCTATAAAGTGGGTGAGAAAAAGTTCTACTGCCTTTCCCTAGACGAGC
AAACCGTAAGGAACGCGGTCATAGACGCGGGGTACTCTATAAAAGACCTG
GAACTATATTATTTACCTAATCCCGCCTCTTGTGCCCACATCACCGATAC
CTATGCAAATATCTTCCTTGTGGCGCAGAAGAACGAGACC

Seq. ID No: 77

>INMT_10ATGGAAATCGTGTCTACCTCATATAACCACATCTACGACAAC
TTTGATGCAAGGAAGTACCTGGACAGATACTATGGGTTGGCATCTGAAAC
CCAAGAAATTGAGGAAGAGAGTGTTTTTCTTTTAACCTTTCTTAGTAACG
TATTTTCTAGCGGGAGGGTGAAGGGACATTCTTTTATTGAGATTGGTGTA
GGGCCGTCCATACATTCTATACTTTCAGCATGTGAGGTGTTTGAAAAGAT
TTACCTGACTGATTACTCCCAAGGAAATTTGAACGAAATCGAGAAGTGGT
TGAATAGCGAAAATGATGCATTCGATTGGACTCCCTATATAAGATTCGTG
TGCGACTTAGAGAATAATGGTTCTACTCCTAAGGGCAAGAAAGAGAAGTT
ACGTAGAGCTGTCAGTTTAATGAAGTGTGACGTAAACTTGTCCAACCCGC
TACACCCCCATTCACTTCCACTTACTGATTGCCTACTGACGGCGTCATGC
CTTAGTGCTACCTGCAAAACCTTCACAGATTTTAAGATGTCTCTTAAAAT
CATCGTAAGCCTAATCAAACCAGGCGGACATCTAATCCTTATTGACTATC
TGAGGGCGAGTTATTACTGGGTTGGAGAAGTCAAGCTGCCCATTTTGTCA
TTGGACGAACATGTTGTGAGGGAAGCTGTCGTGGAAAGCGGCTGTAAGAT
AGAAGAGTTCAAATGGTTTAAGGAGTTTCACATGCCTGACGAGTTGTCAG
ACTGTAAAACTGTGTTCTCCTTACTAGCTCAGAAGTTG

Seq. ID No: 78

>INMT_11ATGGATTCTAGCAACTACAAACTATACCACGTGCACGAGTTT
AATTCAAGGTCCTTCCTAGACAATTACTTCTCAGATGGTCCGCAAATGAC
TTTCGTGGACGACACACTTGTCTTTCCAATAGAAAACTTAAAGAAGACCT
TCGCGGAGGGCCACATAAAGGGTGATGTGATGATAGACCTTTCAATAGGT
GCGATGGTACACCATCTTTACGCTGCATGTGAATTTTTCAAAGATATAAT
AGTCTTGAAGGCATCAGATAGATGCATCATGGAACTTAAAAGGTGGGTTG
GAACAAGGACTGGGGCATTTTACTGGGGACATGCCACGAAACTACACGCT
GACACAGAAGGGAACAGTGAACTACTGCAGGATAAAGAAGAGAAGGTTAG
ATCAGCGATCCAACACGTGGTGAAGTGCGACGTAACCAAGGAATTGATGA
CCGATCCGATCGTTCTTCCGCCAGCCGACTGCATCATCTCTGCATGGCTA
CTGGATGCAATCAGTTCAAATCAAGATGACTTCATAACGTACTTGAGAAG
GTTCATAAAGTTATTGAAGCCAGGCGGGCACTTGATCCTTATCGGAGCGT
TGGAACAAACCAGTTATTCAGTAGGCAACGAGAAATACCAGTTCCTTACG
TATAATGAGGACTTTGCGAGGAAAGCTCTTATAGCTGAAGGCTTGGTCAT
CGACGACTGCAAAATCAAGAAAAGAACGGCAAAGTCTGATTTAGCGGATT
ATAAATCCATATTGTATTTAGTTTCACACAAAAAG

Seq. ID No: 79

>INMT_12ATGGACCCGTGTCTTAATCTGTACTATCCATCTCATGAGGTA
AACGCCAAGAGATTGTTACATGAATACTTTTCCCAGAATGTCCCCTACTC
TATATTCAAAGAATCAACTATAAATATCATGAAATGCTGTTACAAGGCTT
TTAGTTCAGGTTTGCTTAGCGGTACAACCCTTATAGACATTAGTGTCGGC
CCGTCCATAGTACATCTTCTATCTGTGTGTGAGTTTGTGGAGGAAATAAG
TATCCTAAAAGTCAATGACGCCTCAATTAGGGAGCTGGAACTTTGGAAAA
ACAAAGATCCTGAAACGTTCGACTGGACTCATACTTTGAAGCTGTTTATG
GAATTGAAAGGTACTAGCCGTGACGGATGGAAAGACGCTCAAGAAATGCT
GCGTAGGAAAGTTAAACACATAGTCAAATGCGATTTCTCTAAATCCAATC
TGACTAAGCCCTTCGCCTTACCTAGGGCTGACTGCGTTACTTGCATTTGG
GGTCTTGAGACTATCAGCAGAGATCACGACGAGTGGAAAACTACCCTTAG
GAAAATTAGTGATCTGGTTAAACTTGGCGGTCATGTGTTGATTCATGCTG
ATATTAACGCTTCATACTTTAAAATTGGTGAGGACAAATATCACTTGTTT
AACTTCGATGATGCGTTTCTTAGGAAAACTCTTACTGACGGTGGGTTCGC
TATTGTTCATTACGAAAATCTGGAACGTGAGGCATGCACCGATTGTTTGG
ACCATTCCCACAAG

Seq. ID No: 80

>INMT_13ATGGAATTAAAGCGTTGGGTCGATACCCGTACAGGGGCATTT
GATTGGAGTCACGCTGCTAAATTGCACGTGGATACCGAGGGTAATTCCGA
CGAGCTTCAAGAGAAGAACGAGAAAGTCAAAAGTGCAATTCAGCATGTTG
TGAAATGTGACTTAGAAAAAGAAAACATGACACATCCTATTGTCTTGCCA
CCTGCCGATTGCATAATCAGTTTTGCCCTGCTTGATGTAATATCAAAAGA
TAAAGACGATTACATTAAATACTTGAGAAAGTTTTCAAAGTTATTGAAGC
CTGGTGGGCATTTAATTTTAATAGGCGACCTAGATACTACGTATATTACT
GTGGGTAAGCACAAAGTTCACTATCTGACATACGATGAGGAGTTCGTGAG
GAATGCCTTAGCTGGTGAGGGATTCGTCATAGACTGTTGTAAAGTCAAAG
AAAGAACTGTCGAGTCTGACCTATGCGATTACAAAGGTATGATCTTCATA
GTCGCCCACAAGGAAAAG

Seq. ID No: 81

>INMT_14ATGGAGCTGAAGAGATGGGTGGACACCAGGACTGGAGCGTTT
GACTGGTCTCATGCGGCAAAGTTACACGTAGATACTGAGGGAAATAGCGA
TGAGCTTCAAGAGAAGAACGAAAAAGTCAAGAGTGCTATACAGCACGTAG
TGAAATGTGACTTGGAGAAGGAAAATATGACACATCCGATTGTACTACCG
CCCGCTGACTGCATTATCTCCTTCGGGTTCCTGGACGTTGTCTGCAAGGA
CCAAGAAGACTATATCAGATATTTGAGGAAATTCAGTAGATTACTGAAAC
CTGGGGGCCATCTGATACTAATAGGGGGTGTTGACGCTACTTATTTTACC
GTCGGGAAGGAGAAGCACCATTTCTTCACATACGACGAAGCCTTTGTGAG
GAAGGCATTAGAGGGGGAGGGATTTGTCATCGATGACTGCAAAGTAAAGA
AGCGTACAGCAGTAAGCGATTTTACCGATTATAAGGGGAGTATATTCATC
GCCGCACACAAAGAACAC

Seq. ID No: 82

>INMT_15ATGAGCGACTTCACCAATACTTCAGAATACGAAGAGCAATTT
GACCCGAGGTTATACTTGGAAACATACTTCCACTTGGGATCTGGGAGCCT
GGCGGATGATTTTCTAAGATTCGTTTTAGACAATTTTAATAAAACTTTTA
AATCTGGGGCGGTGAAGGGATCTACATTAATCGATATAGGCACTGCACCA
TCTATTTATCAGCTTTTAAGTGCGTGTGAATCATTTGATGATATAACAGT
TACATGGCACACTAACAGAGAGTTGAAAGAATTACAGAAGTGGCTTAACA
ATGAAGCTGACGCTTTTGACTGGAGCAGTATTGTGAAGCATGTGTGTGAG
ATTGAAGGTAACAGAATGGGTCAGAAGGAAAAAGAAGAAAAGCTGAAGGG
CAAAATTAAACAGGTGCTGATGTGCGATGTTAGCAAGTCAAATCCTCTGA
GCCCTCATGAGGTTCCGAAGGCTGATTGTCTGCTTACGACCGTTTGTTTA
GAAGCGGCCTGTAAGAATTATGAATCTTATGGTACTGCCCTTAAAAACCT
ATCCAATCTATTGAAGCCAAAAGGTCACCTTCTGATGGCCGGCGATCTAG
GCGCTAATTACTACGAAGTTGGGTCCAACAAGGTATTTAGTCTTCCCGTT
AATGAAAAATTCTTAAAGAAGGTCATCTCAGAGTCCGGGTATGAGATTAT
ACAGCTAGTCAGCTTCGGAAAACCCGAGAACGCTGACTTTGACACTTCAG
ATTATGAAGGTTTCTATTTCGTTCACGCGCAGAAAGTC

Seq. ID No: 83

>INMT_16ATGGCGTTACAAGAGAGGCAAGAACCTGACGTTTATCAGGAA
AACTTTGAGCCAACCTCATATTTGGAATACTACCGTATGAATCAAGATCC
AGTAGGGGACGAGGTATTGCATTTCCTTTTAAAGCACTATAACGCGACGT
TCAAACCGGGAGGGTTAGAAGGGAAACTACTTATTGACATAGGTTCTGGT
CCCACCATATATCAATTTTTATCCGCCTGCGAGTCCTTTCAAGAGATTAT
TGCTACGGATTATACAGATAAGAACTTACAAGAATTGGAGAAGTGGCTGA
AGAAGATGCCTGGCGCATTTGATTGGTCTCCTGTCGTCAAGTATGTTTGT
GAGCTAGAAGGGAATAGAGATAAGTGGGCTGAAAAAGAAGAACGTGTGAG
GAGGGCTGTCACCCAAGTGCTGAAGTGTGATGTGTTAAAAGAAAGGCCAC
TGGAGCCCGCGGTGCTTCCCCCAGCGGATGGTCTGATTTCAAGCCTATGC
CTTGAAGCAGCCTGCCCCACCCCTCAAGCGTGCAGAGATGCTCTGAGGCA
TCTTAGGACACTGCTTCGTCCCGGAGGGCATCTTGTTCTAAGCGGAGGCT
TCGAGACGACATTTTTCATGGTGGGAGACAAGCGTTTCTCAACGTTACCA
TTAAACGAAAAATTCTTGAGGGAGGCACTGCAAGAGGCAGGCTTTATAAT
CGAGAAGCTGGAAAAGGTAACCAGGGCCGCGGAAACCCACCTAGATAATC
GTTCTGATTACACAGGTCTATTCTTCCTGGTTGCGCGTCGTGGAGAC

Seq. ID No: 84

>INMT_17ATGGACAAAATTTCTGCCCCTTTCTTTAGTGGCACATCACCA
GCAGCAGCTTCAGTAGCAGGCGTTGATGAGGACGACAGGTTATGTTTCCA
AGCCCAGGAGCTAATGTTTGCCTACAATATATCTATGGTCTTGAGAGCAG
CAATCCAGTTAGGGTTACTGGACGCACTGTCAGCCGCTGGTGGGAAAGCA
TTAACGCCCAACGAGCTTGTGGAAAACGTTGAGACATCTAGTAACAAAGC
CGAAGCAGCGGCAGCAGTGGACCGAATCCTGAGATATCTATCATGCTTCA
ACGTGGTTACCTGCAGCTCAGAGGCTGCAGGTCCAGATGGAACTCTTGTT
AGGCGTTACACCACGGGGCCCCTTTGCAGGTGGCTTACTAAAGACAGAGG
GGATGGGACCCTGTCTCCTTTCGCTGTCTTTGTCGTAGACCCGGACCACC
TGTTCCCTTGGCATCACATTGCCGAGGCGGTCACCGCTGGGGGTCCATCT
GCATTCGAAAGAACACAAAAATGGCCTTACTACGAATATATGGGGAAAAA
TCAAAGACTAGGTACCCTTTTCGATAATGCTATGGCACAGCATTCTGTCA
TACTTGTTACTAAGATGCTAGAAAGGTTCAAAGGATTTGACGGTGTGCAA
AGACTGGTTGATGTGGGCGGAGGGACGGGTTCAACCTTGGGAATGATAAC
GAGTAAATATAAACACATGACCGGAATAAACTATGATCTTCCCCACGTTA
TTGCGCAGGGGCTACCCTTGCCGGGAGTAGAACATGTAGCAGGAGATATG
TACGAGTCTATACCAACCGGCGATGCAGTCCTTCTTCAGTGGATAACACT
AATGCTTAATGACGATGAGTTCGTAAAGATCCTATCCAACTGTCACAATG
CATTGCCCAAGGACGGGAAAGTCATAGTCGTTGATGGGATCTTACCCGAG
AATCCTGACAGCTCTTTAACGGCTCGTGACGCCTTTACGTTAGATATCAT
TATGTTTGTTCTTTTCAAGGGAGCGAAACAGCGTACCGAGAAGGAGTTCG
CGCGTCTGGCGAAGCAAGCCGGGTTTACGGGAGGGATTAAGAAGACTTAT
ATATTCTTTAACTTCTATGCATTAGAATTTACAAAG

Seq. ID No: 85

>INMT_18ATGGACGCTAACAAGAGATACCACGGTCCGCCGGTGTTGCTG
GGGGTGGTAAGGGACTCAGAGAAGTTTGACTTCTGTATGTGTAATCCTCC
GTTCTTTGAGACAATGGAAGAGGCTGGCCTTAACCCCAAGACTAGCTGCG
GAGGTACCCCAGAGGAGATGATTTGTCCAGGCGGGGAAAAAGCCTTTATC
ACCCGTATAATAGAAGACAGTGCTGTTTTAAACCAATCATTCAGGTGGTA
CACGAGCATGGTTGGAAGGAAGAGTAACCTGAAGAGTCTTATATCTAAAC
TGAGGGAAGTCGGTGTGACTATCGTTAAGACAACGGAGTTTGTACAAGGG
CAGACATGCCGTTGGGGTCTAGCCTGGTCCTTTGTTCCTCCCGTGAGGAA
GATAGTTTCACCGCATGTGGCCGAGAAGAACATCATTAGCTTCATGCTAG
AGGTGTGGGTCCCCGGATTTAGTATATGTAGGGTTGGCGACGATCTTGTT
CCGAAGAGTAAGTCTCCTCATCTGTCTCCCATACTTGGAACGAAGAAC

Seq. ID No: 86

>INMT_19ATGGAAGAAGCAGGCTTGAACCCTAAGACAAGTTGCGGCGGC
ACGCCCGAGGAAATGATCTGTCCTGGTGGAGAAAAGGCCTTCATTACACG
TATAATCGAAGATAGCGCGGTCTTAAATCAGTCCTTCAGATGGTATACCT
CAATGGTTGGAAGAAAGAGCAATCTTAAATCTTTAATCTCCAAGTTAAGA
GAAGTTGGGGTCACAATCGTGAAAACCACGGAATTTGTACAAGGCCAAAC
GTGTCGTTGGGGTCTGGCGTGGAGCTTTGTGCCCCCAGTCAGGAAGATTG
TAAGTCCCCATGTTGCGGAGAAAAATATAATCAGTTTTATGTTGGAGGGA
TTGCAACGTCAATTTTCCGCAATCCACGTGTTGCAATCTATTGAATCTTT
CTTCAGAACTTGCGGGGCAAGCAGCGAGCTAAATGCAAGTTCATTCACTG
TAGATATAACTGCTACAAATGATCATTGCAAGGCGATTTTGAATAATGAG
CTTCAAAGCATAGATGAAGCCACTTCATGTGAACATGTCCCGGAGACCTC
TAATAGCAGTTCTTCCCTACATCCGCACTCTAATGGTCTTGGGTTTAGGA
TCAGCGTGTACCAGCAAATTCCGGGGACGTTGTTAGTTAAGGGCTCATTA
CAGCACAAAAACAACCCAGTGAGCGGGGCCTTTTCATTGATAATTCAGAG
GCTTGAAGAGGACCTTAAATACAAGTTTTGCAGA

Seq. ID No: 87

>INMT_20ATGAATAGGAGCAATTATATTCATTGGATTGAGGACTTGTTA
GCGTCTGACATAACCGAAAAGAACGAAGCGAACGGAGGCAAGGTAAGGGG
GTTTGACATTGGAACGGGAGCGAACTGCATATATCCACTGTTGGGCGCTT
CCCTGCTGGGTTGGAGCTTTGTAGGATCAGATGTCACTGAGGTAGCCCTA
GATTGGGCTGAACAGAATGTAAGGAGTAACCCACATATCTCAGAGCTGAT
AGAAATCAGGAGGGTGGATGTCGATGATCCTGCGAGTTCCTCAGGGACCG
TAGAGTCATCCGGTGGCAGTAGAATGGAAGACTCTTCACAGGGTCAGTGT
GACGTAGTAGAGTTGGCATCCCTTGAAATGAAGGAGTTCTGTGACGTTGG
CGTTACCTGTAAGGGAGGAACTGATAAGAACCAAAGGAGATACGATGAAG
CAAAGCACTCAAACGTAGCTAAGGGCTACCAAGGTCCTCCCATTTTGCTG
GGGGTAGTCAAAGAGGGAGAAAAATTCGATTTCTGTATGTGT

Seq. ID No: 88

>INMT_21ATGGAAGAGGCTGGATTAAATCCTAAAACCTGCTGTGGGGGC
ACACCGGAGGAAATGGTTTGCCAGGGCGGAGAGAGGGCTTTTATTTCCAG
GATTATAGAAGATTCAGCCACGCTAAAACAAAGTTTCAGGTGGTACACGT
CAATGGTGGGTAGAAAGAGCAACTTGAAGTTTTTAATGTCAAAATTGCGT
GAAGTGGGCGTAACCATTGTTAAGACGACCGAGTTCGTACAAGGACAGAC
CTGTAGGTGGGGTTTGGCATGGTCTTTTATGCCAACCGCGAAGAGATCCG
TGCCATCTCATGTCGCTGAGAAGAGAAATCTGTCTTTTATGTTAGAAGGA
CTGCACCGTCAGACCTCTGCATTCAACGTCTTACAATCAATGGAGTCCTT
TTTCTCTCATTTCGGAGCACTTTGTAAGAGTAACCCTTCTAGCTTCACTG
TTGACGTGTCAGTATCATCTGACCACTGCGACGCAATCTTGAAATCTGAC
GTGGAGAAACTGGACGAGGCATCATCCCACTCCTGCGTCGCAGAGAGTCC
GGGTTCTGCGTCAAGTTACGACCCTATGGTCGTGTCATTCCGTTTGTCCG
TGTTTCAACAAATTCCTGGTACCTTGTTGGTCAGGGGGTCTCTACAACAG
CGTGATTCACCATTATCTGGGGCATTTCTGTCTGTATTCCAGCAGTTAGA
AAAGTTCTTGAAACACAAGTTCTGTAGAGAGAGAGGACTTCAGTTTAACC
AACGT

Seq. ID No: 89

>INMT_22ATGGCCACGGAAATAGATGACGAGTCATACGAATCAGCTAGG
AGAAACATTAGCAATAATAATATGCAGTCCCGCATTCACGTGGAGAAGGC
GAGTCCCGACCAATCCATTTTGTTCCCCCTGGAAGATGACAGAACGTTTG
AGTTCACCATGTGCAACCCGCCATTTTACGGTAGCGCCGCTGAGGTGGTA
CAGTCCGCCGAAGCTAAGGAATTTCCTCCCAATGCGGTTTGTACAGGAGC
GGACATTGAAATGATTTACCCCCACGGAGGAGAAGAAGGTTTCGTCATGA
AGATCCTGGATGAATCCGAGAGGTTCATGACTAGGTGTAAATGGTACACC
AGTATGTTAGGCAAGATGTCCAGCGTCGCTACTATTGTTGAAGTTTTGAG
ACAAAGATCCATTACGAATTATGCCGTTACGGAGTTTGTACAGGGTCAAA
CCAGGAGATGGGCTATCGCTTGGTCATTCGCAGACACAAGGCTGCCAGAT
ACTATGGCAAGAATACAGAGCATCAGCCCGAAACACGCGCTATACCCGTG
CATGCCACCCAAAAATACTTTGGTTCAGGCTTTCCCGGGACCTGCGACCC
ACCTTGTCAGCACGAAACTAATTGAAACTTTGCACGGCATAGAAGGGGTA
TCCTACACGACAACTAGCCTCAATTCTTTCTTCGTTGAAGCCAGACAAAA
TACTTGGTCTCGCAGCGCCAGGAGGTCTCGTGCTAATAAGAATAGTTCGA
AAAAGCCCGACCCATCCAGTTTAGACGCGGATGACATTCTGTCCGGGAGT
CAACCTGCACTTACCTGCTCATGCAGAGTGCTGGCGGATACCGCACATGC
GGACCCGGTGAACGTTGTCGAAAACCAATGGATTTTCGGTAATGACAGAG
CCTTATTCGAATCCTTTGTGGGTCATGTTTCCCGTAAGGTAGGAATGGGC
CTACGTGACGTTAAA

Seq. ID No: 90

>INMT_23ATGCTTCTGGAGAGCTACAAAACTTTCGAGCCCGCTAACTAT
TTGCAAGAATACTACTCTACTGTAGACTTAGAGAATCGTTCATTGTTAGC
CTTTTTCGCAGAGGCATATAAAGGCATCGACCCTAACAGTGTTATGCTAG
AGTTTAGCGGAGGGCCATCTCTTTATTCTCTAATCACCGCGGCGGCTCAC
GTTAAGGAAATACATTTCAGCGATTTTCTGGAACGTAACGTGGAAGAGAT
AAAATTGTGGAAACGTTTCCGTCACCGTTCCTACATTTGGATTAACTTCT
TTAAGGAAGCGTTAATGGCCGAGGGGCTATCAGAGGTGTCTACTGATGAT
ATTCTTGAGCGTGAGGAACTATTAAGTAAAAAGCTATCAGATTTCCTTCT
ATGCGATGCGTTCAATAGGCATCCTTTGGGTCAGAGATGCTACCAAAGAT
ACGACGTCGTTGCAGCAAATTTCGTAGCCGAATCCATAACCCCATCCTTG
AAAACTTGGGAAGAAGTGGTCAATAATATATGTAGTACTTTGAAGCCGTC
TGGAACCTTAATAATGACCGCGATCCAAGGGGCTAGTTTCTACTGTGTCG
AAAATCACAGATACCCCGCGATTGCCGTTACGCCAGAAGATGTCATCAGG
GTTTTATCATATCAAGGCTTCGACGTAGATAACTTATTAATGCGTCATAT
ACCGGCGGAAATCACAGACATCTCAGCGAAGGACTACAAGGGCTATCAAG
GCATGTTATTCGTCAAGGCCACGAGA

Seq. ID No: 91

>INMT_24ATGGAAAGTGGATTCACTTCCAAAGACACCTATTTGTCTCAT
TTCAATCCCAGGGACTACTTGGAGAAATACTATTCATTTGGTAGCAGACA
CTGTGCAGAGAACGAAATTCTACGTCACTTGCTGAAAAACCTTTTTAAGA
TATTCTGCTTAGATGGCGTTAAGGGAGAGCTGCTGATAGACATAGGAAGT
GGCCCAACCATTTATCAATTGTTGTCCGCTTGCGAATCATTCACTGAAAT
AATCGTAACAGACTACACAGATCAAAATCTATGGGAACTACAGAAGTGGC
TTAAAAAGGAGCCCGGTGCTTTTGATTGGAGTCCTGTAGTTACGTACGTG
TGCGACCTTGAAGGCAATCGTACTAAAGGGCCGGAAAAGGAAGAAAAGCT
GAGGAGAGCCATTAAACAGGTCCTTAAATGCGACGTGTCCCAAAGTCAAC
CCCTTGGAGGGGTCTCTCTTCCACCCGCAGATTGTCTATTAAGTACACTA
TGCCTAGACGCTGCCTGCCCAGATTTACCGGCTTACAGGACCGCCTTACG
TAATTTAGGATCTCTACTTAAACCGGGCGGGTTCTTAGTCATGGTTGATG
CACTCAAGAGTTCTTACTACATGATCGGGGAACAGAAGTTTTCCAGTCTT
AGTTTGGATAGAGAAGCAGTGAGGGATGCTGTGGAAGAGGCTGGTTACAC
AATCGAACAGTTCGAGGTCATATCACAATCCTATAGTTCTACAACTAGCA
ACAACGAAGGCTTATTCTCCCTGGTCGGCCGTAAGCCCGTGGGGAGCGAG

Seq. ID No: 92

>INMT_25ATGGAGATCGTCAGCACTTCATACAACCATGTGTATGAAAAC
TTTGATGCAAGAAAATATCTTGACAGATACTACGGCATTGCGCCCGAAGC
CGAGAAAATAGATGAGGAATCCGTGTTCTTGTTAACCTTCCTATCCAATG
TATTCTCCTCAGGGCGTGTCAAAGGTCACTCTTTCATAGAGATTGGGGTC
GGGCCTAGTATCCACTCCATCCTTTCTGCTTGTGAAGCGTTCGAAAAATT
GTACTTGACTGACTACTTCCAGGGAAACCTGGATGAGATTAAAAAGTGGT
TAAACTCTGAGAACGACGCATTCGACTGGACGCCATACATTAGATTTGTT
TGTGATTTGGAGAATAATGGGAGTACACCGAGGGAGAAGAAGGAGAAAAT
CCGTCGTTGCGTATCCCTAATGAAGTGTGACGTGAACCTAAGTAACCCGC
TTCATCCCCACAGTCTTCCTTTAACGGATTGTCTTCTGACCGCTTGCTGC
CTTACCTCTACTTGCAAGACGTTTACTGATTTTAAAATGAGCTTAAAGAC
GATTGTCTCCCTAATAAAGCCCGGCGGCCATCTAATTCTTATAGACTATC
TGCGTGCTAGTTACTATTGGGTAGGCGAAGCCAAATTGCCGTTACTGAGC
TTGGACGAGCATGGAGTCAGGGAGGCCGTGGAAGAATCTGGATGTAAGAT
CGAAGAATTTCAATGGTTCAAGGAGTTCCACATGCCTGACGAGGTCAGTG
ATTGCAAGACGGTGTTTATACTATTAGCTCAAAAACTA

Seq. ID No: 93

>INMT_26ATGAGGAACCTGCACGAGACTTTTGGCCCCGGCGGCGTCAAG
GGAGATATTCTAATTGACTTCGGGGCTGGTCCGACGATATATCAGTTGTT
ATCAGCGTGCGAGGTATTTAACACAATTATTACCTCAGACTTCTTAGAAC
AAAATCGTGAACAATTGAAAAAGTGGTTAAGGAAAGATCCAGATGCACTA
GATTGGAGTAATTTCGCCAAATACGTTTGCGAATTGGAGGGTAAGTCCGA
CAATTGGGAGAAGAAGGAAGAGACGCTGAGACGTAAGGTCACTAAAGTTC
TGAAGTGTGACGCCTTAGCAGAGAAGCCTTATGACCCGGTCCCAATGCCT
GAGGCGGACTGCCTGATATCTTGCCTATGCTTAGAAGTGGCCTGCAAAGA
CCTTGAGGATTTC

Seq. ID No: 94

>INMT_27ATGGAGACTCCTTTTACTTCACAGCAAACATACGTCGATGAG
TTCAAAGCATCAGATTACTTTAAAACGTATTACGTTGCAGAGGGCGGCAT
TGCCAATGAAGAATGGACCGATTTTGCACTAAGGACTCTGCATGAGACTT
TTACGAAGGGAGGTGTGAAGGGCGAAACGCTTATAGATTTCGGCGCTGGC
CCTACTATCTATCATCTACTGTCAGCGTGCGAAGTGTTTGATAAAATCAT
CACTAGCGACTACCTTGAGCAGAACAGAGCCGAGCTTGAAAAGTGGCTGA
AAAAGGACCCGAGTGCCTTTGATTGGACACCAATAATCAAGTTCGTCTGC
GAGCTAGAAGGAAACAGGAATTATGAGAAAAAGGCTGAGAAGTTGAGGAA
TAAGGTAAAAGAAGTTCTAAAATGCGATGCACTTAAAAGAAATCCGTTTG
ACCCTATTGTGTTGCAGCCGGCGGACTGTTTACTAACTTGTCTATGCCTT
GAAGCACCATGTGAGGATATGAAGAGTTACTTTAACGTGCTAAAGAATTT
CAAGGATCTGATCAAACCAGGCGGACATTTGGTTATCCTATCTGTTCTTG
ACGCGACGTTCTACTATGTTGGAGACAAATACTTCAGCAGCATGACAACA
AGGAAAGAGGAACTGGAACAAGCCTTGAAAGAAGCCGGTTTTGAGATTGA
AAAAGCCGTTTATACCACAAGGAAGGACAGATCACAGATGGATATAGCGG
ATTATCAGGGTTTTTACTACATACATGCGCGTAACCCCAAG

Seq. ID No: 95

>INMT_28ATGGAGGGGAGCTTTACCGGAGGTGAGGAGTACCAAAAATAC
TTCCAGCCGCGTGACTACCTTACTACATATTATAACTTCGATGGTTCCCC
TACCCCAGAAGCGGAAATGTTGAAATTCAATTTGGAATGTTTACACAAGA
CCTTCGGACCTGGTGGTTTAAGGGGAGATACCCTAATAGACATTGGGTCA
GGACCAACGATTTACCAAGTCCTTGCCGCTTGCGAGAGCTTTCGTGACAT
TACTCTTAGCGATTTTACAGACAGGAACAGAGAGGAGCTGGAAAAATGGC
TGAAGAAAGAGCCTGAGGCTTACGACTGGTCATCCGTTGTAAAATTCGCC
TGTGAGCTTGAGGGTGATAGCGGGCGTTGGCAAGAGAAAGAGAAGAAGTT
GAGATCAGTGGTTAAGAGGGTCTTGAAATGCGATGCAAATTTGGCGTCTC
CACTTGCACCTGCAGCCCTTCCACCCGCCGACTGCGTTCTTACGTTACTG
GCGATGGAGTGCGCCTGTTGTTCACTGGACGCATACAGGGCTGCATTATG
CAATCTGGCCTCACTTCTAAAGCCCGGAGGTCACCTAGTGACCACGGTGA
CCCTAGGCATTTCATCCTATATGGTTGGCAAGAGGGAATTTTCCTGTGTA
GTCCTTGAGAAGGAGGGTGTGGAACAAGCTGTCTTAGACGCGGGTTTTGA
CATCCAGCAGTTTCTACACATCCCTAAATGTTACTCTGCAACTATAGCCG
CGAACAATGGTGTCTGCTTTATCGTGGCTAGAAAGAAGCCTGCCCCC

Seq. ID No: 96

>INMT_29ATGGAGGGGTCCTTTACGGGACCCGATGAGTATCAAAAATAC
TTCTCACCTAAGGATTACCTTGATACTTACTATAGTTTCGAACATGGACC
ATCACCCGAGACCGAAATGATAAAATTTTCTCTTCAATTCCTTCATAAAG
TCTTCGGGCCAGGTGGCATCCGTGGTGAGACCCTGATAGATGTCGGAAGC
GGCCCGACGATATATCAGGTCCTGGCGGCCTGTGAGGCGTTTTCTGACAT
AACTCTTTCCGATTTCACGGACCGTAATAGGGAAGAACTACAAAAGTGGC
TAAGAAAGGATGCCGGAGCTTTTGATTGGACTCCCGTGTTAAAATTCGCT
TGCGAACTTGAGGGCAACAGCTCACACTGGCAGGAAAAAGCAGAAAAGCT
AAGGGCAACTGTCAAGCGTGTATTGAAATGTGACGTTAACCTGGGTAAGC
CTCTTGCGCCAGTCGAGTTGCCAGCGGCGGATTGTGTCCTTACGCTTCTT
GCAATGGAGTGTGCCTGCTGTTCTCTGGCCGCATATAGGGCGGCGCTTTG
TAATCTAGGATCTTTGCTGAAACCAGGAGGTCATCTAGTAACATCCATTA
CACTTCAAATATCCAGTTACATGGTTGGAAAACACCAGTTCTCTTGTTTA
TATATAACAAAAGAAGAAGTGGAGAGGGCAATCCTGGACGCAGGTTTCGA
TATTGAACAGTTACTTCACAGTGAGCAGAGTTACAGTGCTACGATCGCTC
CAAATAAGGGTATTTGTTTCATTGTGGCTAGGAAGCGTTCCGGTCCG

Seq. ID No: 97

>INMT_30ATGGACGCCCAGCTAACTCAACTGAGGAACGCTGATGTAAGC
TGGGCGGCTTTCGACCCAATCGCTTACGTTGACCACAACTATAGGGATCT
ACAAGCGGAGGATGCTGAAATTCTTCATCTTGTTAGGGATCACTTCGGTG
ACCACTTTAGGAAACAGGGTGGGGGACCAGTCTCAGGAATAGATGTCGGT
GCAGGCGCCAACCTTTACCCCGCTTTGGCAATGATGCCCTGGTGTGAAGA
GATAACCCTGTTTGAGCGTAGTCCGGCCAACGTACGTTATCTAAAGAGTC
AAGTGGATAGCTATGATGCCAACTGGGATCAATTCTGGGACGCCTTGTGT
GCCCATGAAGCATATAATAGCCTGGGTACTGACCCAAGAGAGCGTTTCGG
GAAAGTAGTCTGGGTAGAACAAGGTGACCTTTTCGACCTAGCGAGATACG
AAAGGAGGTGGTCAATGGGCACTATGTTTTTCGTAGCGGAGTCAATGACT
ACATCATATCAGGAATTTATGTTAGGAGTCGAGAGATTCATGCGCGCGCT
AAGCCCTGGAGCACCCTTTGCGGCTGCGTTTATGGAACATAGTAAAGGTT
ACCATGCGGGGGAGCATTTCTTCCCGGCGTGCGACGTGGGTGAGAGCGAG
GTCAGAGCGTCTCTTGAGGGGTTTGCCGGAGACTTTAAAGTGCAGAGGTT
GGAATCCGCAGCGCAGCTTCGTGATGGATACAGTGGTATGATCGTGGCAT
AT

Seq. ID No: 98

>INMT_31ATGTCAGATTTTACAAACGCCAGTGAGTACGAAAAGCAGTTC
GACCCGAGACTATACCTTGAAACCTATTTTCATTTAGGTTCAGGGTCATT
AGCAGACGATTTCCTTAGGTTCACTTTGGGCAATTTTCATAAGACCTTCA
CAGAGGGCGAGGTGAAAGGCACGACATTAATTGACATTGGAACAGCCCCT
TCAATCTATCAATTGTTGTCAGCCTGTGAATACTTTCAGGATATCACCGT
AACGTGGTATACCAATAGAGAACTACAAGAGCTACAGAAATGGTTAAATA
AGGACCCCGGCGCCTTTGACTGGTCTTCCACGGTAAAACACGTTTGGGAG
CTAGAGGGTAAACGTGGGATGTTGGAGGAGAAAGAGGAGAAACTTCGTGG
TATGATTAGACAAGTGCTTTTATGTGATGTGTCAAAGAAGAACCCTTTAG
AACCCGTTACTTTGCCGAAAGCGGACTGTCTAATATCAACCGTGTGTTTG
GAAGCGGCGTGCCGTAACTACGACTCTTATCGTACAGCCCTAAAGAATCT
AAGTACGCTTCTAAAGCCAGGTGGACACCTTTTGCTTGCGGGGGACTTAG
GAGCCAATTACTACGAAGTTGGTTCTAATAAGGTTTTCAGTCTTCCCGTC
AACGAGACTTTTCTAAGGAAGGCCGTCAATGAGAGCGGTTATGTCATCAA
CAAACTTGTGTCCTTCGGAAAGCCTGAAGACGCAGGGTACGACACTTCCG
ATTATGAAGGGTTTTACTTTATTCACGCTCAAAAATGT

Seq. ID No: 99

>IOMT_1ATGAGTAGTAAGTTAGACAATCAAAATATTACCGCTAATGAAG
AAGAGGAGGCTTTCCACCAGGCCATGCAGCTGGCCATGTCCACTATTTTA
CCGATGGTCCTGAAAGCCGCAATAGACCTAGACCTGTTAGAGATTATAGC
GAAAGCTGGACCGGCGGGATGCAAACTTTCTCCTATAGAGATCGCTAGTC
ACCTTCCCACCAAAAATCCAGATGCGAGCAGCATCATCGACAGGATTTTA
AGAGTTCTGGCCTCACATTCTATCCTGACATGTGATCTTGCTACCAACGA
GGACGGTCACGTACAACGTTTGTATGGTCTGGCCCCGATTGCGAAATATT
TCTTACACAATGATGATGGGATAAGCCTAATCCCCACTTTGACCATTTCA
ACGGATAAATACCTACTTGGTGCGTGGTATCATCTACGTGAGGCAACGTT
GGAAGGCGGGGCAATCCCACTGGTCAAGGCGTATGGAATGGACTTATTTG
AGCTGGCGGCGAAGAACGACGAAATAAGTGGGAAGTTCAACAACACAATG
GGTAACCAAACAGCGATTATCATGAAAAAGGTGTTGGAAATTTATAAGGG
ATTTGAAGGCATTAACCAGTTGGTGGACGTGGGAGGCGGACTTGGTATCA
ACTTGAAATTGATCGTATCCAAGTACCCTCAGATCAAAGGTATAAACTTC
GACCTACCGCACGTAGTAAAGGACGCTCCTCATTTCTTAGGCGTTGACCA
TGTGGGTGGTGACATGTTCATCGAGGTTCCACAGGGAGAGGTAATATTCA
TGAAGTGGATTCTGCACGACTGGGGAGACGATAGATGTCTAAAGCTGTTG
AAAAATTGTTATAATGCTCTACCAAAATTCGGAAAAGTAGTTGTGGTAGA
GCTAGTGGTGCCTGAATCTCCCATGACAGACATCGTCACAAAGAATACGT
TAACGTTGGATGCAGGCCTATTCATTGTGGTTCCAGGTGCCAAGGAAAGG
ACAAAAGAGGAATACGAGGCACTAGCCAAGAAGGCGGGCTTCTCTACGTT
TAGATTGGTATGTAGGGCCTATTCTTATTGGGTGATGGAGTTCCACAAAA
ACGTGATAGTG

Seq. ID No: 100

>IOMT_2ATGGGAAGCCAAGCCGAGGTTGGGAAGGCGATGACGGAGGAAG
AGGCTTGCGAATTTGCAATGCAGCTAGTCTCTTCCAGTATCTTGCCTATG
ACTCTTAAAGCAGCTCTTGAGCTTGAGCTTCTTGAAATTATGGCAACGGC
TGGTGAAGGAGCCCAACTGACTCCAGCGGAGATTGCTGCTCAACTTCCTA
CCTCTAATCCAGACGCCCCTATAATGCTAGACAGGATGTTAAGACTTCTT
GCCTGCCACTCTGTATTAACGGCATCAACTTATACCGACGACGATGGGAA
GGTCAGAAGACGTTATGGCTTGGCGCCTGTATGTAAGTTTTTAGTCAGGA
ATCAGGATGGTGTTTCTACGGCCGCGCTATCCTTGGTGAACCAGGATAAG
GTGACGATGGAGTCTTGGTATTACTTAAAAGACGCCGTACTGGAGGGCGG
TATTCCATTTAACCGTGCTCATGGGATGACGGCTTTCGACTATCCGGGGA
CTGATCCTAGGTTCAATAGGGTGTTTAACCAGGGAATGTCTAACCACTCA
ACCCTGACCATGAAGAAAATCCTAGAGACTTATACTGGCTTTCGTGGCCT
TCACTCCTTAGTCGATGTAGGCGGTGGCATCGGAGCTATTCTTTCCCTAA
TCGTCGCCAAATTCCCTCATATCAAGGGGATTAATTTCGACCTACCACAC
GTTATAGACGATGCGCCACAATTCCCAGGTGTTGAACACGTGGGAGGCGA
CATGTTTGCATCCGTGCCAACAGCCGAGGCCATTTTATTAAAGCTAATTC
TACATGATTGGGGTGACGAACATTGTGTGAAATTGTTGAAAAACTGCTGC
AAAGCGTTACCGGAAGATGGAAAGGTAGTCGTAGTGGAGGCCATTCTACC
AGAAGGGATTGATCACTCTTATGCGTCAGCTTGCGTTTATCAAGTCGATA
TGATTATGTTAGTCACAAACCCCGGGGGAAAGGAAAGGACCTTGAAAGAG
TTTGAAGAACTTGCCAAGGCCGGAGGGTTCGCTGGCATAAGGCCAATTTG
CTGCGTCTATGGAAGCTGGGTGATGGAATTTTACAAGAAGATG

Seq. ID No: 101

>IOMT_3ATGGGATCAACAGCAGAAACTCAACTGACCCCCGTACAGGTCA
CCGATGACGAGGCTGCCTTGTTTGCAATGCAATTAGCATCCGCATCCGTC
CTACCAATGGCGTTAAAAAGTGCACTGGAGCTGGACCTGTTAGAGATCAT
GGCGAAAAATGGGTCTCCTATGAGCCCGACTGAAATTGCTTCCAAGCTAC
CTACTAAAAACCCCGAAGCGCCTGTAATGTTGGATAGGATACTGAGGCTA
TTGACAAGCTATTCCGTCCTTACCTGCTCAAATAGGAAACTTTCTGGTGA
TGGCGTTGAAAGGATATACGGATTGGGACCGGTCTGTAAATATTTGACGA
AGAATGAAGACGGAGTGAGTATCGCGGCGTTATGCCTGATGAATCAAGAC
AAAGTGCTGATGGAATCCTGGTACCACTTAAAAGATGCAATACTGGATGG
CGGCATCCCGTTTAACAAGGCATATGGAATGTCCGCGTTTGAGTACCACG
GGACAGACCCACGTTTCAACAAGGTCTTCAACAATGGCATGTCAAATCAT
TCAACGATCACAATGAAAAAGATACTAGAGACCTACAAGGGCTTTGAGGG
GTTGACTTCATTGGTGGATGTTGGTGGTGGAATTGGGGCGACGTTAAAAA
TGATTGTGAGTAAATACCCTAACCTAAAAGGGATCAACTTCGACTTGCCT
CACGTCATTGAAGATGCACCGAGTCATCCTGGCATCGAGCACGTAGGAGG
GGATATGTTTGTTAGCGTCCCGAAGGGAGATGCTATTTTTATGAAATGGA
TATGTCACGATTGGAGCGACGAACATTGCGTTAAGTTCCTGAAGAATTGC
TACGAATCACTTCCTGAGGACGGCAAAGTGATCCTTGCCGAATGCATTCT
ACCAGAAACTCCTGACTCCAGCTTATCTACTAAACAAGTTGTACACGTAG
ACTGTATAATGTTGGCTCATAACCCCGGGGGTAAGGAAAGGACCGAAAAG
GAATTTGAGGCTTTGGCAAAGGCATCAGGATTCAAGGGAATTAAAGTTGT
TTGCGACGCATTCGGTGTGAATTTAATCGAGCTATTGAAGAAACTA

Seq. ID No: 102

>IOMT_4ATGGGCTCCACAGCTGCAGATATGGCGGCTAGTGCCGACGAGG
AGGCTTGCATGTACGCTTTGCAGCTGGTCTCCAGCTCTATATTGCCCATG
ACATTGAAGAATGCCATAGAGTTGGGCTTATTAGAAACTTTGGTCGCAGC
CGGGGGAAAGTTATTAACCCCAGCAGAGGTTGCGGCAAAACTGCCCTCAA
CCGCAAACCCTGCCGCTGCCGATATGGTTGACCGTATGCTAAGATTGTTA
GCCTCCTACAACGTTGTTTCTTGCACTATGGAAGAGGGGAAGGACGGCAG
ACTAAGCAGGAGATACCGTGCGGCGCCCGTTTGCAAATTCTTGACTCCCA
ACGAGGATGGTGTAAGCATGGCAGCCCTAGCTCTAATGAACCAGGATAAA
GTATTGATGGAGTCTTGGTACTACCTTAAAGACGCAGTCTTAGATGGTGG
TATCCCGTTTAATAAGGCTTATGGCATGTCCGCGTTTGAGTACCACGGAA
CGGACCCTCGTTTCAACAGAGTGTTTAACGAGGGTATGAAAAATCACTCC
ATTATTATAACAAAGAAACTGCTAGAAGTGTATAAGGGCTTTGAGGGATT
GGGAACTATCGTTGATGTTGGGGGTGGAGTCGGAGCCACTGTTGGAGCGA
TAACAGCCGCTTATCCAGCGATTAAAGGCATCAACTTTGATCTTCCGCAC
GTCATCTCTGAGGCACAGCCGTTCCCAGGAGTGACACACGTTGGCGGGGA
CATGTTCCAAAAAGTGCCTTCAGGGGACGCGATTCTGATGAAATGGATAC
TTCACGATTGGAGCGATGAGCACTGTGCTACCCTGCTGAAGAACTGTTAT
GACGCACTGCCCGCACATGGAAAGGTTGTACTGGTGGAGTGTATATTACC
GGTTAATCCTGAGGCGACGCCTAAGGCGCAGGGGGTTTTCCATGTAGACA
TGATAATGCTGGCTCATAATCCGGGAGGCCGTGAGCGTTACGAGAGAGAG
TTTGAAGCACTGGCAAAGGGAGCCGGGTTCAAAGCCATAAAAACAACCTA
CATCTACGCCAATGCCTTTGCGATCGAATTTACTAAG

Seq. ID No: 103

>IOMT_5ATGGGCTCAGCTGGGGAAACCCAGATTACCCCGACTCATGTGA
ATGACGAAGAGGCCAACCTTTTCGCTATGCAGTTAGCAAGCGCGAGTGTG
TTGCCGATGATCTTAAAATCTGCGTTGGAACTGGACCTTTTAGAGATAAT
CGCGAAGGCCGGGCCCAATGCGCAGCTATCTTCTAGTGACATCGCTTCAC
AGCTGCCTACCAAGAATCCAGACGCGGCAGTGATGCTGGACCGTATGATG
AGGCTGTTAGCTTGCTACAACGTATTGAGTTCTTCTTTGAGAACACTTCC
TGATGGAAAAATCGAACGTCTATACGGTTTGGCTCCAGTTGCGAAATACC
TGGTAAAGACGGAAGACGGTGTGTCTATCGCACCACTGAGTTTAATGAAT
CAGGACAAAGTGCTGATGGAAAGTTGGTACTACCTAACCGAGGCGGTGTT
GGAAGGCGGCATCCCATTTAATAAGGCGCATGGTATGACCTCCTTCGAGT
ACCACGGAAAAGACGCTAGATTCAATAAGGTGTTCAATAAGGGGATGGCT
GACCATAGTACGATTACAATGAAGAAGATTTTGGAGACATACACAGGGTT
CGAGGGACTAAAGTCTTTGGTCGATGTGGGAGGCGGCACTGGTGCAGTTA
TTAGCATGATTGTAAGTAAATATCCGTCCATCAAAGGGTTCAACTTCGAC
CTACCACATGTGATAGAGGAAGCACCGTCATATCCCGGCGTCGAACACGT
AGGTGGGGATATGTTTGTGTCAGTTCCGAAGGCTGATGCTGTTTTCATGA
AGTGGATCTGCCATGACTGGAGCGACGAGCATTGTGTAAAATTTCTGAAA
AATTGCTATGATGCTTTACCTGAAAACGGAAAGGTGATCGTGGCAGAGTG
CATCCTTCCAGTTGCCCCAGACTCTAGCCTAGCAACAAAGGGCGTCGTCC
ATATCGATGTCATAATGTTAGCCCACAATCCTGGAGGGAAGGAGAGAACC
GAGAAAGAGTTCGAAGCTCTAGCAAAAGGTGCGGGGTTCCAAGGGTTCAG
GGTATGTTGCTCTGCTTTCAATTCATATATTATCGAATTTTTGAAGAAAC
CG

Seq. ID No: 104

>IOMT_6ATGGGATCTACTGCGGAGACCCAAATCACTCCAGTCCAGGTGA
CCGATGATGAAGCTGCTTTGTTCGCCATGCAGTTGGCGTCCGCATCTGTT
CTACCTATGGTCTTGAAGTCTGCGTTGGATCTTGATCTGTTAGAAATAAT
GGCGAAGAACTCTTCACCGATGTCCCCGTCCGAAATCGCGTCTAAGCTAC
AAACCAAAAACCCCGAGGCACCAGTCATGTTGGATCGTATCCTACGTCTA
TTAACCTCCTACAGCATTCTTACATGCTCAAATCGTACGATTCTGGGTGG
TGACAGTGTGGAGAGAATTTATGGTCTGGGACCCGTCTGCAAATATCTTA
CAAAGAACGAAGACGGTGTCTCCATAGCCGCTTTATGCCTAATGAATCAG
GATAAGGTACTTATGGAATCATGGTACCACCTTAAAGACGCTGTCTTGGA
TGGCGGCATACCTTTCAACAAAGCCTATGGCATGTCAGCCTTTGAGTATC
ACGGCAAAGATCTGAGGTTTAATACAGTTTTTAACAATGGAATGAGCAAT
CACAGCACGATCACTATGAAGAAGATTCTGGAGACATACAAGGGCTTCGA
GGGTCTAACTAGCTTGGTTGATGTTGGGGGCGGAATAGGTGCGACTTTAA
AAATGATAGTGTCCAAGTACCCGGACCTTAAAGGCATCAACTTTGACTTG
CCGCATGTGATAGAAGAAGCGACCAGTCATCCTGGGATTGACCACGTAGG
TGGCGACATGTTCGTTAGTGTGCCAAAAGGAGATGCCATATTTATGAAGT
GGATATGCCATGACTGGTCCGACGAACACTGTGTTAAATTCCTTAAAAAT
TGCTACGAAGCGTTGCCCGAGGACGGAAAAGTTATCTTGGCAGAGTGCAT
TCTGCCCGAAACACCCGACTCATCTCTTTCCACAAAGCAAGTCGTCCACG
TAGACTGTATAATGTTGGCCCACAATCCAGGGGGAAAAGAAAGGACAGAA
AAAGAGTTTGAAGCGTTGGCAAAGGGCTCAGGTTTTAAGGGAATCAACGT
GGCATGTAATGCATTTGGCGTGTATGTGATAGAGCTGTTGAAGAAAATG

Seq. ID No: 105

>IOMT_7ATGGAAATGATAAACTTCATGCATCATATGGATTCAACGTGGA
ATCTTTGTGGTAAAGATGTAGTGCAAGCCTTTGACTTTTCTGAGTTCCAC
ACGGTTTATGATCTAGGTGGCTGTTCAGGCGGCCTAGCGAAGCAGTTCGT
CTCCACGTATAACGATTCCACGGTAACGATTATGGATCTACCAAAGGTAG
TACAAACTGCCAAGAAGTACTTCGTGACGGATCAAGAACAACAGATTCAC
TTCATCGAGGGCGACCTTTTCAATGACCCCATCCCTGAGGCGGACCTGTT
CATTATGGCCAGGATAATCCATGACTGGACAGAAGAGAAATGCCTAGAGC
TGCTGAGGAAAATTTACCAGTCCTGTCGTCCAGGTGGTGGTGTGCTGTTG
GTCGAAGTTCTTTTGAATGAGGATAAGTCTGGACCCCTAATGAGCCAATT
ATTCAGCTTGAACATGCTAGTTCAAACAGAGGGCAGAGAGCGTACACCAA
GTGAGTACACAAAACTTCTGACCGACTCAGGCTTTCGTGACATTCAAGTG
AAGATTACTGGGAAAATATACGATGCA

Seq. ID No: 106

>IOMT_8ATGGAACGTCTACTTGATGCCTGTGTGGGCTTAAAACTTTTGA
AAGTTGAGCTAAAGTCCAACAAGGGTTACTACAGTAACACAGATGTCTCT
ACCATGTACCTTGTAAAATCTAGCCCCCGTACTTTATACTATATGATAAT
GTTCTACTCCAAGACTACTTACATGTGTTACAACTTCTTACCTCAAGCAG
TCAGGGAGGGTCAATGTCAGTATGAGAGAGCCTTCGGCATATCCTCTAAA
GACCTATTTGAGGCTCTATACCGTTCTGAAGAGGATACTTTAGCATTCAT
GTATTTCATGAACAGCACATGGTCTATTTGTGGAAAGTATGTGGTCCAGG
CGTTCGACCTGAGTGAATTTCACACAATTTATGACTTAGGCGGCTGTACG
GGCGCTCTAGCTAAGCAGTTAGTCAGTACGTATAAAGAGAGCACCGTCAC
TATTATGGACATGCCAAACATCGTTCAAGCCGCCAAAAAGCACTTTGTTA
CTGACAAGGAGCAGCAAATTCATTTTCTTGAAGGCGACTTCTTTAATGAT
CCAATACCAGAAGCT

Seq. ID No: 107

>IOMT_9ATGATCCCATTCAATAAGGCTTACGGTATGACCGCCTTTGAGT
ACCACGGAAAGGACGATAGATTTAACAAGGTCTTTAACGCTGGCATGTTT
AATCACTCAACGATGACCATGAAGAAGATCCTGGACATCTACGACGGCTT
CAACAACCTAACGACGCTAGTAGACGTGGGAGGAGGAACTGGGGCTTCTC
TAAATATGATCGTGAGTAAGCATCCCAGCGTAAAAGGTATTAATTTCGAC
CTTCCACACGTCATTCAAGATGCCACTACTTATCCGGGCATAGAGCACGT
AGGAGGTGACATGTTTGAATCTGTTCCAAAAGGTGATGCCATCTTTATGA
AGTGGATCTGTCACGATTGGTCAGATGCCCACTGCTTAAAATTTCTTAAA
AATTGCTACAAGGCGCTACCAGACAATGGTAAAGTCATTGTAGCC

Seq. ID No: 108

>IOMT_10ATGGCACAAGCCGCGGCGGAGGCCGAAGGCATCACCCCGGTC
ATGGACTTACTATTCGCAGCACAAGGGTCCTCTGCCTTGCTAGTCTGCGC
TAGGTTAGGCCTATTCGATTACATCTCCAGCCAGGGTGAAGAAGGGGTGA
GCTGCAAGCAACTGGCGTCCAGGGCGCAGTGGTCCACACGTGCTGCAAGT
GCCGTTATGGTTAGCCTTGCTGCGAGTGGAATACTAGCTGTAAAACCTAG
TTCAGCCGGCGCTCAACACTGCTTTGAGCATTCCTACACACTTACGCCTA
GGGCCCAAAGGTTCCTGGTTACCGAGAAACCTGGGAGCATGAGCGCGTAT
ACCGAGATACACTGGGAAGCATCCCCGGAACTACTTCTGAAAAAGGCCGC
TGAGACTGAGGACGAAAAACGTAACTTTATGCTAGAGACAGGGGGAGGAG
CCCCCTCTGAGGTATTCCTAGCGGCGATGCAGGGCCAGAGTAGTTACGCT
GCGATGGTGTTGACAAGGTTAGTAGACTTATCGGACACCAGGACATTCGT
CGATGTAGGCGGTGGAAGTGGCACTTTCGCAATAGAAGCCTGCAAGGCGA
CCCCCAACCTTCAAGGAGTTGTGTACGACCTAGCCGGGGCATGTCCTACC
ACCGACGGTTTCATTGCAAGGGCAGGTATGGCTGAGAGGGTGAAGACACA
TGCGGGGAACATGTTCGAGGATGAGAGGTTCCCTGCGGCTGACTGTTATG
CATTCGGGAATGTGCTTCATGATTGGTCAGATCAAGATAATAGCAAGTTA
CTTAGGAAAGCATTTGAGAGCCTTCCTGCCCAAGGTAAAGTGCTTCTACT
AGAGATGCTGGTGGAAGAAGACGTAGTGAGTACCTCACCCTCTGCGGCCG
GACTAAATTTATGCATGGTGACTAACGAGCTGGGGAGACAGTTTAAAGCA
AGTGAACTACGTGCAATGTTGCTAGAGGCAGGGTTTGCAGGGGCCGAGGT
CGTTTCATCACCTCTGACTCCTTACTCCCTAGTCGTGGGAACGAAAGGTG
AGGCGAATCCTGTGGCGAGCAAACCCGAGGCCGCTGCGGCTGCGGAATCA
GAGAGCATTACTCCGCTGATGGATGTGTTGTTTAGCGCCCAACACAGTGC
TGTACTGATAGTTTGCTCTAGGTTAGGCGTGTTTGATTTTGTTGGCGCGC
AGGGCGAATCAGGTGCTTCTTGTGCTCAGGTGGCGGCACATGCAAAATGG
ACCACCCGTGCGGCTTCAGCGATGCTTGTTAGCCTAGCTTGCAGCGGCCT
GCTGGAGCCTACACCGGGAAGCGCAGCGGCCCAACACTGCTTCGAACATA
GCTATAGGCTTACTCCTCTAGCTAGGAGGTTCTTGGTAGCTGGTCAGCCA
GGACAACTGAGTGCTTACACTGAAATATTTTGGGGCGCTAGTCCCAAACA
ATTATTAGAAAAGGCGTCCGCTTCACTGGGCGAGTGGGGCGAGGGAAATT
TTATGTTAGACGCCGAGGGCGGAGCCCCTTCAGAGGTATTTCTAGCAGCC
ATGCAGGCGCAGTCAACCTACGCCGCAATGGTTCTTACACGTTTAGTCGA
TCTTTCTGATGTGCGTACATTCGTTGATGTCGGTGGAGGGTCAGGTACTT
TGGCCATCGAGGCCTGTAGAGCCGCTCCAGGCCTACAGGGTGTCGTGTAT
GATCTGGCAGGTGCGTGTCCCGTTACCGACGGTTTCATTGCCCGTGCTGG
AATGGCAGAACGTGTGAAGACTCACGCCGGCAACATGTTTGCTGATGAAA
GATTCCCCGCCGCTGATTGTTATGCATTTGGTAACGTGCTGCACGACTGG
TCAGACCAAGACGATGGAAAATTACTAAGAAAGGCGTTCGAAAGTTTGCC
TGCGAACGGTAAGGTCTTGTTATTGGAAATGCTATTAGCCGAGGACGTGG
AGTCTTCAACGAGGAGTGCAACAGGACTAAACATTGTAATGGTGACCAAC
GAGCAGGGGCGTCAGTTCAAGGGCTCCGAGCTTGAGGCTATGTTACGTGC
TGCTGGTTTTGCTGCGACAGAAGTGGTTCGTTCACCACTAACTCCCTACG
CATTGGTGGTTGGTACTAAAGGC

Seq. ID No: 109

>IOMT_11ATGAGCAGGACATCATGGGACGAAGGCGAGGATGTCGATCTG
GATTCAGTTGCTTATGGTTTCATGGCAAGCCAAGCATTGTTTACTGGTCT
GGAGCTTGGCATTTTTGATCACATCGCTGCAGCGGGAGCTGGTGGCTTGT
CAGCGGCTGGCATCGGGAAGGCTTGTGGGATAGAAGCGCCCAGGGTTCAA
ACGCTGCTTACTTCATTGGTCGCAGTTAAGTGCCTGAAGCGTGACGCATC
TGCCATGTACACCTTGAGTCCAAATACGGCACAATATATGGTAACTAGCA
GTAGACATTTTTATGGCGACTATTTAAGGTATCAAATAGGGCGTCAGTTT
TACCACCGTATGGGGGCCTTGCCTGAGGTGATGACGTCTGGTAAAGCCCC
GAGTTACGCATCATGGTTTAGTGATCCTGAAGTAGCTAGAACTTACACAC
AAGCACAGCATAACGGTTCCGTTGCCACAGCCAAGTATTTGATAAAGAAG
AAATTGCAACTAGGGGGCATATCAGCGATGTTAGACGTCGGCGGGGGTTC
AGGGGCGTTTTCCTATGTCTTTACTCAGGCCACACCGGGTTTGCACAGTA
AGGTACTGGAACTGCCAGAGGTCTGCCGTACCGGTGAGGGTATCCGTGAG
AAGCAACCCGAGGATGTCCGTAGCAGGGTTAGCTTTGTCGAGTTAGATGC
GAGTAGCCCGACGTGGCCGGTTGATGACTCTGCGTTTGACGTCGTGCTAA
TGTCCTATATATCAGGCTCAGTTCCCGAGCCGATCATTGGATCATTATAC
GCCAACGCTATGAAGGCTTTAAGGCCCGGAGGACGTCTTCTTGTGCATGA
CTTTATGGTCAACGACTCACTAGACGGACCGGCGTTAGGGGCTCTGTGGG
GATTGCAGCACGTCACAGTCAACGCGGACGGTTTGGGACTGTGCCCAAAG
GAGGTTATCGCGCGTATGGGAGCAGCAGGCTTTGACACAAGTAAATGTGA
AGCAATGGAGATGATTCACGGGATGACTAAGCTGATAGTCGGACACAAGG
GC

Seq. ID No: 110

>IOMT_12ATGTGCTCTAGCAAGGAACTAGATTTTCCGCATATCTTGATT
GATTACCAGCACGGCTTTCTAGTATCCAAGACGATATTCACGGCGTGTGA
ACTAGGCGTATTCGACCTATTACACGAGGTACAAGAGCCCGTGCCCGCTG
CGACTATTGCATCTAGGCTTAGCACATCTGAAGATGGAATGGAGAGATTG
TTAGACGCTTGCGTAGGCTTAAAGCTACTGAAAGTCTACCTGAAGAATAA
TAAGGGCTACTACTCAAATACTGACGTTTCCACTATTTATCTAGTTAAGA
GTTCACCTAAAACCTTACATTACATGATGATATATTACTCCAAAATAACG
TATATGTGCTGGCATTTCCTACCACAGGCCGTTAGAGAAGGTAAAAGACA
ATATGAGAGGGCGCTTGGCACAACGAGTAACGATCTTTTCGAGATTGTAT
ACAGGTCTGAAGAAGAAATGACGACATTCATGCATTTCATGGATTCTACT
TGGAATCTATGCGGGAAAGACATAGTACAGGCGTTTGATTTGAGCGAGTT
CCACACGGTCTACGATTTGGGCGGCTGCTCTGGGAGCTTAGCGAAGCAGC
TAGTATCCACGTACAAGGAATCAACCGTAACAATTATGGACCTACCGAAA
GTGGTTCAAGCGGCTAAAAAGCACTTTGTCACTGACAAGGAACAGCAAAT
CCACTTTTTAGAAGGTGATTTCTTCAATGATCCCTTACCGGAGGCCGACC
TATTTATCGTCGCAAGGATAATACACGATTGGACGGAGGAAACTTGCATT
AAGCTGTTAAAGAAAATGTACCACAGCTGCCGTCCAGGCGGAGGCGTAGT
GATTGTTGAGCTGTTACTAAACGAGGATAAGTCTGGGCCTGTTATTTCTC
AAGTTTACTCCCTATATATGTTGGTACAGGCAGAGGGAAAAGAGAGGACA
CCCAGCGAATACACTAAACTACTAACGGATAGCGGGTTCAAGGACATAAA
AGTCAAAGCGACTGAAAAACTTTTCGGTGCGATCCTGGGACGTAAG

Seq. ID No: 111

>IOMT_13ATGTGCAGCCAAGAGGGAGAAGGGTATAGCTTACTGAAGGAA
TATGCAAATGGTTTTATGGTAAGTCAAGTGCTTTTCGCCGCCTGCGAACT
TGGCGTGTTTGAACTATTAGCTGAAGCCCTTGAACCGCTGGATAGTGCAG
CTGTTAGCTCGCATTTGGGATCTAGTCCCCAGGGCACCGAGCTTCTGCTG
AATACGTGTGTAAGTCTGAAATTACTGCAAGCCGATGTGAGAGGGGGTAA
AGCAGTATATGCGAACACAGAATTGGCCAGCACATACCTTGTGAGAGGAT
CTCCCAGGAGTCAGAGGGATATGTTGTTATACGCGGGGCGTACAGCTTAC
GTTTGTTGGCGTCACCTTGCAGAGGCCGTGAGAGAGGGCAGAAATCAATA
CCTTAAAGCATTTGGCATCCCCTCAGAGGAACTTTTCTCAGCGATTTATA
GATCCGAAGATGAGAGATTGCAGTTTATGCAGGGGCTGCAAGATGTCTGG
CGTTTGGAAGGCGCTACCGTACTAGCAGCGTTCGACCTGTCACCATTCCC
CCTAATCTGCGATTTGGGCGGAGGATCAGGCGCACTTGCAAAGGCTTGTG
TCTCCCTTTATCCCGGTTGCCGTGCCATAGTCTTCGATATTCCGGGAGTT
GTCCAGATTGCCAAACGTCATTTCTCTGCCAGCGAAGACGAGAGAATATC
CTTCCACGAAGGCGACTTCTTCAAGGACGCCTTACCGGAAGCTGACCTGT
ATATTCTGGCCAGGGTTCTTCATGACTGGACAGACGCGAAGTGCAGCCAT
CTGCTACAGAGAGTATACAGAGCATGCAGGACTGGTGGGGGTATATTAGT
TATAGAGTCCCTGCTGGATACTGACGGGAGAGGACCATTGACCACTCTAC
TTTATAGTCTTAACATGTTAGTACAAACCGAGGGCAGAGAGAGGACGCCC
GCCGAATACAGGGCTCTGTTGGGCCCTGCCGGATTCAGGGACGTGCGTTG
CAGAAGGACGGGCGGCACCTATGACGCAGTTCTGGCCAGGAAA

Seq. ID No: 112

>IOMT_14ATGGGGTATGCGGCACCCCAAGCGCGTCAGAGTGACAAACAG
ATCTTCGATATTTACTTCGGCTTTCTTCACAGCTACGCGCTTCTATTTGC
TGATGAGGTCGGCCTATTCGACCTGCTAAGATGTGAAGCATTGACGTTGG
ATCAGGTCAGTATGGCAACATCCCTTCCATCCCGTAGTAGTCAAGCATTA
CTTTCATTATGCGCGTCTTTGGGACTATTAGAGAAAAGAGGAGAGAGATT
CGCCCTGTCTGCCCTTACGGAAGGGTTCTTAGTTCGTGAAGCGGAGACTT
CATTCTGCGGAGTTTTAGCGTCAGCACGTGGTCAGGCTGCCGCCTTCAGT
TACGATTTCTTCAAGGCGTCATTGCTGAAAGGGGAATCACAATTATTCGG
TGGACGTGACCTTTTCGATAATAACGCACAGGACCCTGAGCATTGCGAGA
TATTTACCAGGGCGATGCACTCCAAATCTAAAGGGCCAGCGCAAGCGTGG
GTGGAAAAGATAGACTTGAGCGCACATGCATGCCTTCTTGATGTGGGTGG
AGGGTCTGGCGTCCATGCTATTTCCGCACTAGCGAGATGGCCTAATCTGA
ACGCTGTGGTGTTCGACTTGCCGCCGGTGTGCGCCATCGCCGATACCTTC
ATCGAAAGGTATCAGATGACAGCAAGAGCACAGACTCATGGAGGGGATAT
CTGGTACACGGATTATCCTTTCGCGGATGCACATTTCTACAGCGATATTT
TTCACGATTGGCCCCTGGAAAGGTGTCGTTTTCTAGCGAGGAAATCTTTT
GATGCCTTGCCCAGTGGAGGCAGGATTATTTTACATGAGATGCTATTTAA
CGCACAAAAGACAGGTCCAAGGAATGTAGCCGCTTACAATGCGAACATGC
TTCTTTGGACTCAGGGACAGCAGCTTTCAGAGCCTGAGGCGGCAGACTTG
TTGCAAGCGGCCGGCTTTGTCGAGATATTGGCCTTTCCGACAGGATATGG
GGATTGGTCATTGGTTACGGGGGTGAAGCCC

Seq. ID No: 113

>IOMT_15ATGGGCTCAATAGATGCCCAAATGGCAGCTGTAGAAGAGGAG
TCTTGCATATACGCTATGCAATTAGCCTACACTGTCGTCTTGCCCATGAC
GCTGAAAAATGCCATTGAATTGGGAATGTTAGAGATTTTAATGGGGGCCG
GGGGCAAAATGCTGTCAGCCTCTGAGGTGGCCGCACAGTTACCGTCAACA
ACTACTAATCCAGATGCGCCAGCAATGGTAGATAGGATGTTACACTTACT
GGCATCCTATAAGGTTGTCAGTTGCGAGGTTGAGGAGGGGACTCATTCCC
GTAGATACGGCCCTGCCCCCGTTTGCAAATGGTTTACCAGTAACAAGGAT
GGCGACGGAGCGTCCTTGGCGGCAATGTTACTTTTAACCAACGAGAAGGT
GTTATTGGAAAGTCTAAACCACTTGAAAGACGCCGTCTTGGATGGCGGAC
ACCCATTTCTGAAGGCTCATGGGATGACGGTCTACGAGTACAACAAAACC
GACGCAAGGATGAAAAGAGTTTTCAGCCAAGCCATGAATAATTATTCTAC
GATCATAAATAGAAAATTGGTTGAAATGTACATGGGGTTTCATGACATCG
CCTTTCTTGTTGACGTCGGGGGAGGAGTAGGGACAACGATTAGGGCTATC
ACTAGCAAGTACCCCCACATAAAAGGTATTAACTTCGACCTTCCGCACGT
CATTGCTGATGCGCCGCAGTGTCCGGGGGTACAACACGTCGCTGGTGATA
TGTTTAGGAATGTGCCAAGTGGAGACGCGATTATTCTAAAGTGGATGCTA
CATAATTGGACGGACGAACATTGTACTACCCTGTTACGTAACTGCTACGA
CGCCCTTCCACCCCACGGTAAGGTTTTTATTGTCGAAAATATTCTGCCGT
TGAAACCAGACGCAACGAGCAGAGGTCAGCAAACGAGCCTGTCTGATATG
ATCATGTTGATGCACACGCCGGCCGGCAGGGAAAGAAGCCAGAGGGAGTT
CCAGGAGCTGGGCAAAGCCGCGGGGTTCACGGGCTTCAAAACAACTTACA
TATATGGAAACAGTTGGGTTATTGAACTTACAACG

Seq. ID No: 114

>IOMT_16ATGAGTTTCGACACCCAGCATGCATTGCAACCATATTGGGAC
TTGGCAGTGGCTCCGGTTCAAGCGGACGGTCTTGCGGCGGCACTAGAGCT
AGGTATCTTTGAGGTACTTGCGACTCCCCACACACCAGCACAGCTGGCAG
ATGTTCTTAGTTTACATGGGCCACACACGGCGCTGCTACTTGAGCTGTTA
TGGTCTATGCAAGTCCTAGAGCGTGATGGAGCAGACGCCGACACCGACGC
CAACGCTCTGAGATATCGTTGTACAGCTACGACGTTGCAATACTTCTGCC
GTGACGCCGTAGCTTTCTGCGGTGACGCTTGGCTATACCGTTTACATGCT
CTGCGTCACTTCGCAACTCAATTAAACACTCTTGTACGTGACGGAGGCAA
GGTGACCCCGTATTCAACGGCCAGTGGAGTTAACTGGGCGGCTGCCGCCC
AACAACAGATCGGCCAAGAGCAAAGAGCCGTAACGATGCGTGCTGCGCTT
TGCGTTATGCAACGTGTAGCTCCATTTGCGGATGGCAACACGCCGTTGAG
ACTGTTAGATGCGGGAGGAGGGCCTGGGTGGGTTGCCATAGCCCTTGCGC
AGGCACATGCGGGTGTTCACGGGTGCGTCTTTGACTGGCCCGAGACGGTG
GCAGTGGCAGCAGCGAATATAGCGCATGCTCAGCTGTCCGACAGGCTAGA
GACTCTAGGCGGAGACCTTGATTCCGACGACATCGGTGGCGGATATGATC
TTATTTGGTGTTCCAGTGTTCTTCACTTTGTTCCAGACATGGCCGCTGCA
TTAAGGAAAATGCAGGCAGCACTGAAGCCGGGAGGCGTGCTAGTATGCAT
ACAGGCGGAGATTGCTGCAGCACCAGGAGACGCAGCTAGGGTGTTACCGT
ACTATCTGCCGATGAGAATGTTAGGAAGGACGGTAACGAGACACGGCGAG
TTGGCACAGTTATTGAGAGATACGGGGTGGCGTCAGGTAGAGCAATATGG
GGCGTCAGACTTCCCAATGGCTCCTGTACAGGTCCTGATTGCTCGTGCG

Seq. ID No: 115

>IOMT_17ATGCAATTGGCTTCTGCTTCCGTATTACCGATGGTATTGAAA
AGCGCGATAGAACTGGACCTGTTAGATATTATTGCAAAAGCAGGCCCTGG
GGCATATCTATCCCCAAGCGAAGTCGCCTCTCAATTGCCCACTAGCAACC
CGGACGCGCCAGTGATGCTGGATAGGATTTTGAGACTACTTGCTTCATAC
TCTGTTCTAACATACAGCCTGAGAACGTTACCTGATGGTAGGGTGGAAAG
GCTATACGGGGTTGGTCCTGTCTGTAAGTTCCTGACTAAGAACGAGGATG
GTGTCTCTATAGCCGCTCTTTGCCTGATGAACCAAGACAAAGTATTGATG
GAGTCTTGGTATTACCTGAAGGATGCTGTGCTGGAGGGTGGTATTCCCTT
CAACAAAGCGCACGGTATGACTAGCTTTGAATATCACGGCAAGGATCTGA
GATTCAACAAAGTCTTTAATAAAGGTATGTCTGACCACAGCACGATAACC
ATGAAAAAGATACTTGAGACCTATAAAGGCTTCGAAGATCTAACATCATT
GGTGGATGTAGGTGGCGGAACCGGAGCAGTATTATCTACGATTGTCAGCA
AATACCCATCTATACGTGGGATTAATTTTGACCTTCCACACGTTATCGAA
GACGCTCCCTCTTACCCTGGTGTAGACCACGTCGGAGGTGATATGTTTGT
CTCTGTACCAAAAGGCGATGCAATATTTATGAAGTGGATTTGCCACGACT
GGTCAGATGAGCACTGCTTAAAATTTCTTAAAAACTGCTACGAAGCATTG
CCCGACAATGGCAAAGTTATCGTCGCCGAATGTATACTACCCGTCGCGCC
GGACACGTCTCTTGCCGCAAAAGGTGTGATCCATATCGACGTCATTATGC
TGGCACACAATCCAGGCGGGAAAGAAAGAACAGAAAAGGAGTTTGAAGCC
CTGGCAAAAGGAGCAGGTTTTCAGGGATTTAGGGTCATGTGCTGTGCGTT
TAACACTTACATAATGGAATTTATCAAAAAGTTG

Seq. ID No: 116

>IOMT_18ATGCTGAATCACACAACAATGGTCATTAAGAAGATTCTTGAA
TGTTACAAGGGGTTTGAAACCCTAAAGCAGTTGGTGGACGTCGGTGGGGG
ACTTGGCGTGGCACTAAATCTAATAACCTCCAAGTACCCACACATCAAGG
GAATAAACTTTGATCTTCCGCATGTGGTGCAGCACGCCCCCTCCTATCCA
GGTGTGGAGCACGTGGGCGGAGATATGTTCAAGAGCGTACCCAAGGCAGA
TGCAATCTTTATGAAATGGATCTTGCATGACTGGAGTGACGAGCACTGCG
TGAAACTGTTGAAAAACTGCTATGCTGCGATCCCAAACGATGGAAACGTA
ATAGTCGTGGACGCAGTTTTGCCGAAAATGCCAGAAGTAAGTACGAGCAT
GCGTTGCACGTCTCAACTTGACGTCCTAATGCTGACCCAAAATCCTGGAG
GTAAAGAAAGAACGGAAGAGGAGTTTATGGCGCTGGCTACCAAAGCGGGT
TTTAAAGGCATTAGGTATCAGGAGTGTTTCGTAAATACTTTCTGGCTTAT
GGAATTTTTCAAG

Seq. ID No: 117

>IOMT_19ATGGAGAGAAAGGAGGAGGTCGCGTTACTGAAAGGGCAGGCG
GAAATCTGGCAGCATCTTTTCGCGTTCGCAGACAGTATGGCGCTGAAATG
CGCTGTAGAGCTGAGACTAGCAGACATAATCCATTCTCACGGTGTACCTA
TAACACTTAGCCAAATAGCCTCCGCCATCGACTCACCTTCACCCGACATC
GCGTATTTGTCACGTATTATGCGTTCCCTTGTGTATAAGAAAATTTTCAC
TGAGCATCATCCCAGTGACGGTGGTGAAACAGTGCTGTACGGTCCAACCC
ATACGTCTAGGTGGCTATTACACGACGCCGAGCTGACTCTGGCCCCCTTC
GTTCTAATGGAGAATAATCAATGGCAACTTGCCCCTTGGCACTTCCTAAG
CCAGTGTGTGAAAGAGGGAGGAATAGCCTTTAAAAAGGCGCATGGGTTTG
AAATGTGGGATTTCGCCGCGCGTAATCCGGAGTTCAATAAGATCTTTAAT
GATGCAATGGCCTGTACTACGAAGATTCTTATGGGAGTTTTACTAGCGGA
ATACAAAGACGGCTTTGGGTCAATTGGCTCTCTTGTAGATGTGGGAGGTG
GGACCGGGGAAATGATCGCTGAGATTATAAAACAGCATCCTCACATTAAG
GGTATGAAT

Seq. ID No: 118

>IOMT_20ATGGGCAGTGCGAGTGGTAGCGCAGAGCGTACACAGATGGGC
GAGGACGAGGCTTGTAGTTTTGCTATGACAATAACATCTGGCAGTGTCCC
TCCAATGGTCTTGAAAGCAGTTATAGAACTGGACGTGCTAGAGATTATTA
AAAGAGCTGGACCAGGGGCACACCTATCTCCTGCTGAAATTGCAGCCCAA
CTACCCACCACTAACCCCGGCGCAGCCGCTATGTTAGACAGAATGCTAAG
ACTTCTTGCCAGCTATGACGTCCTTAGTTATTCTCTGCATACTCTTCCTG
ACGGTCGTGTCGAACGTCTATACGGTCTAGCGCCCGTCTGTCAGTTTCTG
ACTAATAACGAGGACGGGGTGACTCTATCTGCACTATCCTTAATGAATCA
GGATAAGGTTCTGATGGAGTCTTGGTACCATCTGAAGGATGCTGTCCTTG
ATGGGGGCATCCCCTTTAATAAGGCTTACGGCATGACTGCCTTTGAGTAT
CACGGAACGGACCCGAGATTTAACAAGGTGTTCAACAATGGAATGAGCAA
CCATTCCACAATTACAATGAAGAAACTACTAGAAAACTATAAAGGTTTTG
AAGGCGTGTCAACATTAGTGGATGTCGGCGGAGGTACTGGGGCAACCTTA
AATATGATAATTTCTAAACATCCAACTATTAAGGGTATCAACTTCGACCT
TCCGCACGTTATAGAAGACGCCCCAACGTACCCCGGCGTTGAGCATATCG
GCGGCGACATGTTCGTCTCAGTACCAAAAGGTGATGCTATCTTCATGAAA
TGGATTTGCCATGACTGGTCCGATGAACATTGTCTTAGGTTTTTAAAGAA
TTGTTATGCGGCCCTTGCAGATCACGGAAAAGTAATTGTTTGTGAGTACA
TTCTACCCGTCGCACCGGAGACTAATCACGCTGCGCGTACCGTTTTCCAC
GTAGACGCGATCATGCTTGCCCATAACCCTGGTGGGAAGGAGAGAACCGA
GCAAGAGTTCGAATCTTTAGCTAAGGGTGCCGGATTTGAGGGCTTTCGTG
TAGCCTTCTTTTTC

Seq. ID No: 119

>IOMT_21ATGGCACTTAACCCACCACACCAAAACAACGTCATGGAGAAA
GAAGATCTATGCTCCTTTGCTTTGAGCATTGCGACCTCCAGTAGTCTTAG
TATGGTTTTAAAAGCAATTATTGAATTAGATATCATAGGTATAATCAATA
GAGCTGGTCCCGGTGCTCACTTGAGCCCCGCACAGATAGCAGCCCAATTG
CCGACTAAAGATCCGGGCGCGACAGCTTCTATGTTGGACCGTATGTTGAG
GGTACTAGCCAATAACTCTATTTTATCATGCAGTTTACGTGCGCTACCCA
ATGATGGACCCATCGAAAGGTTGTATGGTTTGGCCCCGGTCTGCCAGTTC
TTTACGAAACCGGAGGATTTCGGTCCAATGGTATTATTTTCACAAGACAA
GGTTTACACAGACACCTGGCATCACCTGAAGGATGCCGTCTTGGATGGTG
GAAGTGCGTTTAAGAAGGCCCACGGAACGACACTGTTCGAATACCTTGGG
ACAGATATGAGATTTTCCAAGGTTTTTAATGACGCGATGAGTAGTTCCTC
AACCATAACAATGAAGAAGATGCTGGAAAACTATAATGGCTTCGACGGAC
TGTCTACGTTGGTGGATGTTGGAGGTGGAACCGGCGAGACTTTGAACATG
ATAATAGCTAAATATCCTACCATTAGGGGTATCAACTTCGACCTACCACA
TGTAATCAACGATGCCCCCAATTATGATGGCGTTGAACATGTTGTAGGTG
ACATGTTTGTATCTGTACCGAAAGGTGATGCTATATTCATGAAGTGGATA
TGTCATGATTGGTCTGACAAACTGTGTTTGAAGCTACTTAAAAACTGTTA
TACTGCATTACCCAATCATGGGAAAGTTATTGTCTGCGAATGCATCTTGC
CCGTGGCCCCTGAAACGTCACATAGTGCTAGGGTTGCGTCCAATTTGGAT
ATGCATATGCTTGCGTACTGTAGGGGCGGGAAGGAGCGTACCGAACAGGA
GTTCGAAGCACTTGCTAAAGGGGCTGGTTTTGAGAGCTTTCGTGTGGTCT
GCAGCGCCTATGATCTTAAATTATACATGTGC

Seq. ID No: 120

>IOMT_22ATGGCAGAGATACCGACCAGTAGCAATCCTAGTGATGACCCT
GAAACGCAGAAATTGAATGGCAATGAAGAGGACTATGATCATCATCACGA
TGAAGATCCCGAATCAGACGACGAAAATTACGAATACGCATTACAAATCG
CAGAGATGTTACCATTTCCAATGGTAATGCACACAGCCATAGAGCTAGAC
CTGCTTGGTATTATTGCGACTGCGGGGCCAGACAGGCAATTGAGTGCAGC
CGAGATAGCTGCAGCATTGCCCGCAGCCGGAAACCCAGACGCACCAGCCA
TGCTAGACAGGATGTTATACTTGTTAGCTACTTATAGTGTTGTAACTTGC
ACGGCGGTTGATGGAGGAGCCTCCGGGGGAGTCGTCCGTAAATACGGACT
GGCACCCGTTGCTAAATATTTCGTGTCTAATAAGGATGGAGTCAGCCTAG
GTGCGGTAATCTCTCTTAATCAAGACCAGGCGGTACTTGCATCTTGGAGT
AAACTTAAAGAAGCGGTGCTAGAGGGAGGTATTCCCTTCAATAAAGTCCA
CGGGATGGACGCCTTCGAGTACCAAGGTACTAACCCGAGATTCAATGAAA
TCTTCAACAAGGCCATGTACGATCAATCCACATACATAATCAAAAAGATT
GTGCGTCGTTACAAGGGGTTTGAAAACATCCAGCGTCTGGTGGACGTTGG
TGGTGGCCTAGGCCATACCTTACGTGTAATTACAAGCAACTATCCGTCTA
TCAAAGGGATTAACTTCGACCTACCTCACGTCATTCAACATGCACCGACC
ATTCCGGGGGTTGAACACGTTGGCGGGGATATGTTCGAGTCAATCCCGCA
CGGTGACGCCATTTTTATGAAGTGTATTCTTCATGATTGGTCAGACGAAC
ATTGCTTAAAAACGTTAAAGAACTGTTACAAAGCTCTTCCGAGGAAGGGA
AAGGTCATAGTTGTACAGATGAACATGATTGAGGAGCCCCAGACTACCCC
TCTAGCCAAAGCGATCAGTCAAATGGACCTATGGATGATGACACAAAACC
CCGGCGGGAAGGAAAGGACCAGGAGAGAGTTTCAGGCTCTTGCAGAAGCC
GCAGGATTCGCTGAGTTCAACCCGGTCTGTCATGTGGCTGGGTTCTGGGT
TATGGAGTTTCTGAAA

Seq. ID No: 121

>IOMT_23ATGTCACCCATAGACCTGGCGAATGAACTGCAGACGCTAGTG
ACAAGTACCTACTCCGGTGACGTAACCGACCCCTTCAAACTTTATAAAGC
TAAACATAGCATTAGCGACCTGTGTCTAAGTCTATTGAGGGCAGTTCAGG
GGCCGGAAGAATATACTGCCATATTAGCCGAGAGCTGCCAGGAATCCAGT
GCCCTTAACGTAGTGGCCTCTCTGGGAGTTGCCGACCATATTGCCGAGAG
CCCTAACGGAGAACTGACTCTACAGGAGTTAAGCGAAAAAGTGAAAGCGG
ATGAGAAGTATTTAAGCGTTGTTTTAAGCTCATTGGTGTACCACGGTTAT
TTCAAAGAAGTTGGAGGCTTCGGATCTCAAGTCTATGCGAACAATGATTT
CAGCTCTTTGCTACTATCCGAGGAAACGAATGCGAAGGGCGGCAAGAGTA
TGAAGGACGCGATCGGTTTGAGCGCGGATGACGGGGCGAAAGCTACTACG
AGATTGTTAGATGCGGCAACCGGCAAGGCAAAGGGGGAGGCAAAGACCGC
CGCGAATATAGCATTCGACTTTTCTGAATCACTTTTCCAATGGATGGCCA
GTCCTGGCAATGAATGGAGGGGTAAACGTACGGCCAAGGCTATGGTTCAA
TTACATGGGATGGCCAATGGAGGAATCGGAGAAGACTACCCTTGGGAGAA
ACTGGCGACGCCTATTATCGACATTGGTGGTGGGATAGGATCATTTCAAG
GCATGTTGTTGGCTTTACCAAAGAACAAAGAGTTAACTTTTACAATCTTT
GACATTGAGAAAACGGTTGAGCATGCCAAGAAAGTCTGGGCCGGTAAGCC
TCAATGGATGCAAGATAAGGTAAGTTTTATCGCTGGCGATTTCATGAAAT
CCTCCCCAAATGATAGTAAAATACCAACACCGGCTCAGGGTGCCGGCACC
TACGTGATTAGACACGTACTACATGACTGGGATGATGCCCAAGTAGTCAC
AATATTAAAACACGTCAGGAACGCAATGCTTGGGAGTCCGGCAAGCACAC
CGCCTAAGTTACTGCTTGTGGAGATGATGTTAAACGAAACATCTTCTAGG
TTCACCAGGACCACGTCCTTACAACTGTTAAGCCTAAACGGGGGTATAAC
GAGGACTGAGGTTCAATTCAGGAGGTTGATCAAAGAGGCTGGGTTCACGG
TTGATAGTGTAACGGAAGTGAGGGGTGTTGACCTGGTGGTGGAGTTATCC
CCTGCGAGCTTA

Seq. ID No: 122

>IOMT_24ATGCCTTCTACCACAATTTCCCAATTGGTGGGTCTAATACAA
CAGTCCGTCATGGCGTTAGAGAAGCTATGCTTGGAGAATAGGACGAGTTT
GCCTGACCTAGACGCATTCCACTTCGATCAGTCCTCCGAAACCTTCAGGA
GCCTGCCCGGTGCCGCACAGGACGCTAAGATAGCAGTAGCCGCGTGCATG
CAGCTAATCGCGATTCTAAGTCCCCCAACCGATACAGTTTATAGAGCAGC
TCTTGGGGGTCACCTTTCTTTTGCGACCAGAACATGTCTGGAGGCTAACA
TTACAGAGATTCTTAGGGAAGCGGGACCCGAGGGATTACATATAAATGAT
ATAGCTTCTAAATGCGGGCTAGACCCGTCCAAATTGGGAAGAGTGATTAG
GTATTTGGTTATTCATCATATATATAGAGAAGTCAAGCCCGACGTGTTTA
CTAATAACAGAACATCTTCAACAATGGATACTGGTAAGCCACTAGACAAA
CTTATCTCAGAACCGGATAGAAAGTATGACGACACTGGGTTTCCGGCATT
GATTAGTCATTTTATGGACGTGGACCAGAAGTGCGGAGCCGTGGGCTGGG
ACGTATTAAAGGACCCAGTTCTAGGCCATTCATGTGATCTTACAGAAACG
ATATTCAGTAGGGCTTTCAACACAAAGTCAAAGTATTGGGACTTCTTTGA
CCATCCCGAAAACCATTATATGCGTAGGCGTTTCGACTATGCTATGAAGG
GACTGGGAGCGATCGAAGATCACGATATGGTGCTACATGCTTTCTCATGG
GAGGATCTTGACAAAGGATCTGTTATAGTAGATGTTGGCGGAGGTATTGG
AACGGCCATGCTACCCCTGGCCAGGAAATATCCTAATTTCGATATCGTCA
TCCAAGACCTTCCGATAGTAATCGAGGAAGGCACTAAATTCTGGAGTCAA
AATTTACCAGACGCCGTTGCCAATGGAAACATTAAATTGCATGCACATAA
CTTCTTTGACGAGCAACCTATTAAGAACGCATCTGTATTTTACTTACGAC
ACGTATTGCACGACTGGCCAATGCCCGACATGGTCAAAATATTGAGGCGT
CTTCGTGACGTTGCCGCCGCGAACACGACGCTGATTATATTAGACTACAT
CTTACCCTATTCCTGTAAGATGTTTGCTGACAAGGACGCCGTTTCAATCG
CCTCCGCTCGTTATTATAGCGAAGCGCCGGAACCACTGTTACCAAACTAT
ACCCATAAGAATGTCATAAGCGATAGTGATATGTATGTTTTTCAGATGAT
GTTCCACTACAATTCACAAGAGCATACCTATCTAAGCCTTAAATCCTTAC
TAGACGCTTCCGGCTGGCGTTTAGTCCGTCTGCGTGCGATCGATCCCAGG
AATGACTATTTTCAATCAATTGAGTGCAAGATTCTAGCC

Seq. ID No: 123

>IOMT_25ATGGCCCAGCCCATGATGCTGGCCCTGGCAAAGTTAATATCC
GACAGTGTTGCAAAAGTAGACCAGCTATGCATTGAACAGGGTGTCATTTT
CCCAAGTCTGGACGACCCTTTTACGACGGAAAGTGAGTCCATTAAGTTAC
ACCCAGATGTTGCAGAGGCCTCAAACTATATTATATCCGCGGCGGCTCAG
CTAATCGCAATACTGAGACCTGTGCCCGTCACCTTATCTACGAGTGCTAT
CCATGTGCATGTTTCCTCAGCTCTACGTGTGGTTGTGGATTCTAATGTCG
TCGAGATTCTACGTGAGGCTGGGCCTCAGGGACTGCATGTGAAGAAGATT
AGTGAGAAAAATGGCGTGGAAGCAGGTAAATTAGGAAGATTGTTAAGGCT
TCTTGCGTCCGGCCATATGTTCAAAGAGATCACTCCTGATGTGTTCGCGA
CAAACAGAATTTCAAGTGCTCTGGATACCGGCAAGCCTTATGAGGAGCTA
GTCAAAAATCCGGGCGAGAAATTAATCGGGACAAATGGGATCGCTGCCTA
TATATCAAGATCAACAGATGAGTCCGTCAAGAGCAGTGGGTTTCTGTATG
AGGCTCTGACATATAGCTCAAGCGAGAAAGTACCCCTTCCTCCGTCACCT
TTTAACCTAGCGTTTAACACGGAATTGCATATCTTTTCCTGGCTTGCACA
AAAAGGCAATGAACATCGTTTGCAAAGGTTCGGAATTGCGTTTGACGGCT
TTGACAAGATGTTGCCCGTTAACGGTGTGACCAAAGGCTATAGGTGGGGC
TCCTTGCCAAAGGGTTCTATTGTCGTGGACGTGGGCGGTGGTGTTGGAAG
CGAATCAATGAAGATCGCCAAGACATTTCCAGATCTGAAGGTTATAATAC
AGGATGCTGAAGGCGTCGTAGCAAATGGAGTAAAATTTTATGAGACCCGT
TTCCCAGAAGGGCTATCCTCCGGCCAGGTTACGTTTCAAGCACACGATTT
CTTCACGCCGAATCCTGTAACCAACGCGAGGGTTTTCTTCATGAGGTTTG
TGCTGCACGATTGGCCTGACGCCACCTGTGTCAAGATACTTAAAAACCTG
AGGGCGGCTGCCGCGCCTGATACAGAACTTATCATAAACGAGTGTCTAAT
CCAGTACGCGTGCAGTACCGAGTCAGAAATTTCCAAGTCAATTCCCGGTG
GTAGGTTCAAACCCCCACCTTCCCCGTTGCTGCCAAATTTGGGCTATGCA
CGTATTTTTCATTATCTTATTGATTTACAGATGGCGATAGTTGCGCATGG
AGTCGAGAGGACTGTTGAACAATATGCGAGTATCCTTCAGAAAAGTGGAT
GGAAGCTGAAAGAAGTTCTGAGGATGCCTGAGTCAGCCTATAGCTTACAC
AAGCTGGTAGCCGTCCCCCAGCCTGAG

Seq. ID No: 124

>IOMT_26ATGACGAGGCTAACCGACTCTTTAGGGATGCTAAGAAGCAAA
CTTGTACCTCCACAGGCTACAATGCTACAGCTATTAACGGGATATCGTGT
TTCCCAAGGGATCTATGTTGTTGCAAAGCTGGGCATCGCCGATCTTTTAG
CAACAGGTTCCAAGACCAGTCAGGACCTAGCGGCGATAACTAACGTTCAC
GCTCCAAGCCTGTATCGTCTTATGCGTTCATTGGCCAGCCTGGGAATCTT
TACAGAAACTGAGAATGGGAGGTTTGAGCTAACTCCTCTGGCCGCTACCT
TGAGGTCTGACCATCCAAACTCCGTTCACGATGCCGCTATCATGTTTCTG
GAAGACTGGCATTGGCAGGCTTGGGGCAATTTCTTTGACTGTGTAAAGAC
TGGGGAAACAGCTCTTGAAAAGACATTTGGAACCAGCAATGTGTTTGACT
ATTTTGAGACACAAAACCCCGAAGCTGGGCAGCATTTTGATAACGCCATG
ACGAATACTAGCGTGATGACAAATCAGGCGCTACCTACCGCTTACAATTT
CGGAGCCTTCAAAACCTTGGTGGATGTTGGCGGCGGTCAGGGGTCCTTCT
TATCGGCGCTGTTTCACCAGTGGGATCACCTGCATGGCATACTTTTTGAT
CTACCACCAGTGATTGAAAGCGCAGAGCAGCAAAACTTATTGTCTGGGTT
TGAAAAGCGTACTACACTTGCTGCAGGTGACTTTTTCAAGGCCGTTCCCG
ACGGTGCGGATGCTTATCTGCTGAAGACCATCATACACGACTGGGACGAT
GCCAGCGCGATAGCAATTTTAAAGACATGCAGGCGTGCCATGAATCATGA
TAGTAAGTTACTATTAGTCGAGCTGATAGTGCCGTCCGGGAATGCGCCCT
CCCTTAGTAAAATTTTAGATTTAGAGATGTTGGCAGTTTTTGGTGGGGTC
GAAAGAACGGAAGCCGAGTATCGTTCCCTGTTGCTATCAGCAGGCCTTAA
ATTGACTAGGATCTACGATAGCCCGTGCCCCTGGAGTGTGATTGAAGCTA
TTCCCGTT

Seq. ID No: 125

>IOMT_27ATGTCCATGCCACCGGCCCACAGTCGTTTATACAGCAGGTCC
TTCTTATCTATGCTTCCCGACGCAATCACGCCATTTCCGTATTTGCCACC
TGATGCGACGGATACCAGACCTCTGTTAGCCGAACTGGAAGCCTTGTTAG
AAATAATAAATAGCTCTGCTAGATTGGCCATAACTGAGTACAAAAAGCAC
GGGAATAACGTACCAACGATTTATAGTACAGAATTTCATCCTTTAGATTT
CGCCACGGACACCGTCGCGCTGAAGAAAGCGATCAGGTTGCTTGAAGACG
CATGTCAACAACTTTGTGCATCATTGGCTCCGCCACAGCACACATTAGCG
AACGTGTCCCGTGTGCACCATAGACAGTACGTTACACAGCTAACCACGCA
CGACATCTTGGAAAAATATCCTTCAGGCTCTCATATCAGAGAGCTATCAC
AGACAGTGGGCTTGGAAAAGGGCAAATTGGCAAGGATTTTAAGGGTTTTT
GCTTTCAAGGGATGCTTCATAGAAGTCGATACCGATGTCTTTGCTAGTAA
CAGGTTAAGTTTGATAATGAAATCCAGCAATGACTGTGGATGTCTAACTT
GTATTCACGCGCAAGACGTATCACAGGGCGCTGGTGTCCTTTACGAAACG
TTGACGGAACCTGAATACGCAATGAGCTACGAGCCCGACAAAGCTCCAAT
GATTTATGTCCTTAAACGTAAGGGCTTAAAGGGATCGTTCTTTGATTGGA
TGAAAGCCGATGCCAAAAGGAGGGAAAATTACCATTATGCCATGATCGCT
TTAGGCCCCGTTATGGGTTCTCTTAGCATCCTGCACCACTACCCCTGGAA
TGACGTGGCCACGGTATGTGATGTAGGAGCCAGCGTAGGTAGTGTTTCCA
TCCCCCTTAGTAAAGCTCATCCTCACCTTAAAATCACCGACCAAGATCTG
CCTGAAGTGTTAGAAGCCGCCAGATCCGTATGGGAAAAGGAGGCATTCGA
AGCGTTGCGTGAAAAGAGGGTAGAATTTCTGACGCTTGACTTCTTTAAGG
AGGCGCCCGTCCCAGGCAAAGATGTATACTACCTAAGGCACATCATCCAT
GATTGGCCAGATGCCGAAGCAGCTGTTATCCTTAGGAACATAAGCAAAGC
AATGGAACCGCATTCAAGATTGCTTATCCACAATTATGTAATCGCTGGAG
CCAACCGTAGACCAGATGAAGAACAACGTGCGCCCGAGCCAATGCTACCT
AATTTTGGGGCGGGGGATAGTAGAAAGTACAGGCAAGACCTAAATATGTG
GATTCTGCATAACGCTAAAGAGAGGACGGTCGATGATCAAATCACACTAG
CC

Seq. ID No: 126

>IOMT_28ATGGCCCCTGGCCGTGAGGGGGAACTGGATAGAGATTTTAGA
GTGCTAATGTCTCTAGCGCATGGGTTCATGGTATCACAGGTCCTATTCGC
CGCATTAGACTTAGGCATCTTCGATCTGGCTGCCCAGGGCCCTGTCGCTG
CCGAGGCAGTTGCGCAAACGGGTGGCTGGTCACCAAGGGGGACACAGCTG
CTTATGGATGCTTGCACTCGTCTAGGGCTACTTAGGGGTGCGGGCGATGG
TTCTTACACTAACAGTGCGTTAAGTAGTACCTTTCTGGTGAGCGGCAGCC
CGCAATCACAAAGATGCATGCTACTTTATCTTGCAGGCACAACTTACGGG
TGTTGGGCGCACTTAGCTGCTGGGGTCAGAGAGGGAAGGAATCAGTACAG
TAGGGCGGTCGGTATTTCAGCTGAGGACCCCTTTTCTGCTATTTATCGTT
CTGAGCCAGAGAGACTACTGTTTATGAGGGGCCTGCAGGAAACGTGGTCC
CTGTGCGGAGGTCGTGTCCTAACCGCGTTTGACCTGAGCAGATTTAGAGT
GATATGCGACCTGGGAGGGGGTTCAGGGGCACTTGCTCAGGAGGCCGCTA
GGTTATATCCAGGCTCATCAGTATGTGTATTTGATTTACCTGATGTAATC
GCTGCCGCAAGGACCCATTTCCTAAGCCCAGGGGCCAGGCCGTCCGTGCG
TTTTGTGGCCGGTGACTTTTTCCGTTCCCGTCTGCCTAGAGCAGACTTAT
TTATATTAGCAAGGGTTCTGCATGACTGGGCTGATGGGGCGTGCGTCGAG
CTATTGGGCCGTCTACACAGAGCGTGCCGTCCCGGTGGGGCGCTTCTGCT
AGTGGAAGCAGTATTAGCTAAGGGAGGCGCCGGTCCGTTGAGGTCACTAC
TGCTATCTTTAAATATGATGTTGCAAGCGGAAGGATGGGAGCGTCAGGCC
TCCGACTATAGAAATTTGGCGACGAGGGCTGGTTTTCCACGTTTACAACT
ACGTAGACCTGGCGGTCCATATCATGCTATGTTGGCACGTCGTGGGCCCA
GGCCCGGCATTATTACGGGAGTTGGCTCTAACACCACTGGAACTGGGTCA
TTCGTAACCGGTATTAGGCGTGACGTGCCGGGCGCGAGGTCCGATGCAGC
AGGCACTGGCTCTGGAACTGGGAATACGGGGTCTGGGATTATGCTACAAG
GTGAAACTTTAGAATCAGAAGTATCAGCACCGCAGGCAGGTTCCGACGTT
GGCGGCGCTGGTAATGAACCCCGTAGCGGTACACTGAAGCAGGGTGATTG
GAAA

Seq. ID No: 127

>IOMT_29ATGGAGGTTGTACCATCTTGGTTCAAGGAAACTCTTGATAAA
AGTCAATTTTCTGCGCCATATGAATATGCAGTTGAGACAGCAAAACAAAA
AGCACTGGAAGTTGCTAGGAGAATGCATGTTAAACACCTAAAGACCCCGG
ACATCGTAATCGGAGCAGACACCATCGTCACGCTAGAGGGGGCCATATTA
GAAAAGCCGTTTGATAAACAGGATGCTTACAATATGCTTAGTAGACTAAG
TGGGAAGGAGCACAGTGTTTTCACCGGAGTGGTAATCGTCCATTGTCGTT
CCAAGGAAGAGAACCATTTAGAGACGGATATTATCGACTTTTACGAGGAA
ACGAAGGTTAAGTTCGCAGACCTGTCCGAGGACTTGTTATGGGAGTACAT
CGATAGCGGTGAGCCAATGGATAAGGCGGGCGGCTACGGTATTCAATCTC
TTGGCGGAATGTTAGTGGAATCAGTGCATGGCGACTTCCTAAACGTAGTC
GGCTTCCCCTTAAATCACTTCTGTCGTAAGCTCACTGAAATATACTACCC
GCCCCCAAAACAAGCCATATGCCGTGTAAAGCACGATTCTATTCCATATG
TAGAATCATTTGAGAATCTGAGTGATGTCGAGACGGATTGCACCTCTACT
AGCAAAGCATGTGAAGCGAAGAAAGCCGTGCAGGACGGCGTATGCAAGGC
GGATGGTTCAGGTTCAGCTGTTCTTCAAAATGGAATCGAAGAGAGACCCG
TCCATTGTGCCCAGCAATTAAGTAAAATTACACAGCTGCTTGACGGATTT
AAAGCCTCCCAGACTCTATTCGCAGCATCTAAGTTGAAAGTTTTTGACAA
ATTAAAAGATAAGGGGGCGCTTAAAGCGATGGAGATTGCGGAAAAGATTA
ATGCGTCTGTACACGGAACGGAAAGACTACTAGACGCCTGCGTGGCTTTA
GGCCTACTAGAGAAAACCCACCAGGTTTATTCCAATACGGAACTTGCTAA
CACGTATTTAGTGTCAGATGGAGCGTTCTCCATACATGAATATATTACCT
ACTCAAGCGATCATTTATGGTCCCACTTCACTCATTTAGATTCTGCCGTC
GTAGAGGGCGGGGGACAACATCAGACGGCCGTTAAAAAGGCGTGCGATAA
TAGAAATGGGTCTGAGGTAAAGGAGAGATTTATGAGAGCCATGCATTGCA
TGCTTAAAATAACGGCCAGAGATTTGGTCACCGCATTTGATCTGTCAAAG
TATTCAAGCGCCTGTGACTTGGGCGGATGTACGGGCGCCCTAGCACACGA
ACTGGTCTGGACTTACCCTGAAATGAAAGTCAATGTATTTGACCTACCTG
AAGTAATCAAGCATACCAGTCAATTCCAGCCTGAAAGTTTTGACTCAAGT
AGAGTCACCTTTAGTTCCGGGAACTTTATGGAAGATACACTTCCAGAAGC
AGATCTGTATATTTTGTCAAGGGTGCTACACGACCTGCCGGAGGGCAAAC
TTAATCACCTGTTAAAGAAAGTTAGTGAGGCATGTTGTCCTGGTAGAAGT
GCATTGCTGGTTGCCGAGATTGTTTTGGATGAGGACAAGAAAGAAAGTAG
GGGCTTACTTCAAAGCCTTAGTATGGGAGAAGGGAAACAGAGAAGCGGGA
CTGAATACAAGAAGTTGCTAGAAAACCACGGATTCAATAGCGTCCAGATC
AAAAGTACCGGAAATCTATTAGATGCGATTCTTGCAATTAAGACCAGT

Seq. ID No: 128

>IOMT_30ATGGACACAGTAAAGAACCTACAAGCGAGTAACGTCCCCTCC
TCACTGTCTCAGGAGGATGAGGAAGTGTTCACTTCAGGCCTGCATGTATG
TAGTTCCGAAGTCTTTTCTCACGCGCTGTCCAATTGCATACAACTTGGCC
TTTTTGATATAATCGCTGAAGCCGGGCCCAGTGCATACCTGACCGCTACT
GAAATCACTGCACAATTGCCGACTAAGAACCCCGATGCCGTATCAATGAT
AGATAGGATGTTAAGGCTTTTTAGCTGTCACTCACTATTAAATAGCTCTC
TAAAGACCGTAGCAGATGATGTCGTCGAGACTAGGTATGGTTTGTCCCCG
ATCGGCCATCTGTTCGTGAGAAAGAAGGACGGCGTAACAATGGCGGCCTG
TTTTACCGACTACAAAGCGTGGACCGAAGCCTGGCTGCATTTAAAAGACG
CCATATTAGAGGGTGGGAACCCTTATGAAAAAGCGCACGGGGTCCCTATC
TACGAACATATCTCTTCCGACACTGAAAGTGTCAAGGGCTTCAGTCAAGC
GATGGACTCCATCTCATCATTCATTATGAAGAAGGTCCTTGAAAATTATT
CTGGCTTTAAAGGTCTAGGGTCCCTTGTGGATGTAGGGGGAGGGTCCGGC
TTCGCGCTAAACATGATAACGTCCGAGTACCCCTCAATCTCCTGCATTAA
TTTCGACCTACCGCACGTCGTTCAAGAAGCACCTTATCATCCAGGTGTGA
AGCACGTCGGGGGAGACATGTTTCTTGACATCCCTTCTGCTGATGCAATA
ATGATCAAGGAGGTGTTGCACAATTGGGGTAATGAAGATTGTGTCAAGGT
CTTGAAGAATTGTTACGAGGCGTTGCCTAAAGGAGGCAAGGTGATTGTGG
TAAGTCATGTTATGCCGGAGGTAGTCGGCAGCAGCAACGCAGCAGCCAAG
TACGTGTGCCAATTAGACGTTATGATGCTACTGTTTGGCGGGGGTAAAGA
GCGTACCGAGAAGGAGTTTAAAGCCCTGGGAAAGGCTGCAGGGTTTAGTG
GGTTTCAGCTAATATGTTTCGCAGCTTATAATGCCGTTGCCGTGATGGAG
TTTTACAAG

Seq. ID No: 129

>IOMT_31ATGGCCGAGGACGTAGCCGCTGTCGCAGACGAAGAGGCATGT
ATGTACGCCATGCAGCTTGCCTCTAGTAGTATCCTGCCTATGACCCTTAA
AAACGCATTAGAATTGGGATTACTGGAGGTGCTTCAAAAAGACGCCGGGA
AGGCATTAGCCGCTGAAGAAGTGGTTGCTAGACTTCCCGTTGCACCGACC
AATCCGGCCGCGGCGGACATGGTCGATCGTATGCTGCGTCTACTGGCATC
TTACGATGTCGTTAAATGCCAAATGGAAGATAAAGACGGTAAGTACGAAC
GTAGGTATAGCGCGGCGCCCGTGGGTAAGTGGTTAACGCCTAACGAAGAT
GGCGTCAGCATGGCCGCATTGGCACTAATGAACCAAGACAAAGTTTTAAT
GGAGAGCTGGTATTATCTGAAGGATGCTGTTCTCGACGGAGGGATACCGT
TTAACAAAGCCTACGGTATGACGGCCTTTGAATATCACGGTACGGACCCA
AGATTTAACAGGGTGTTCAACGAAGGTATGAAAAACCACTCTGTCATTAT
TACCAAAAAGTTGTTAGAGTTCTACACGGGCTTTGACGAGAGCGTCTCCA
CCCTAGTAGACGTCGGAGGTGGCATAGGCGCCACATTGCACGCAATCACT
TCCCACCACAGCCATATCAGGGGTGTCAACTTTGATCTTCCTCACGTGAT
TTCTGAGGCGCCACCGTTTCCTGGAGTCCAGCATGTTGGGGGTGATATGT
TTAAATCTGTACCCGCAGGCGATGCGATCCTTATGAAATGGATTCTGCAT
GACTGGAGCGATGCTCATTGCGCTACCCTGCTGAAAAACTGCTACGACGC
CTTACCAGAGAAAGGCGGGAAGGTAATTGTTGTCGAATGCGTACTTCCCG
TAACGACCGATGCAGTCCCGAAGGCCCAGGGAGTGTTCCACGTTGACATG
ATCATGTTGGCTCATAATCCAGGGGGCCGTGAAAGATACGAGAGAGAGTT
CAGAGACTTAGCCAAAGCTGCAGGTTTCTCCGGCTTTAAGGCAACGTACA
TATACGCAAACGCATGGGCAATAGAATTTATAAAA

Seq. ID No: 130

>IOMT_32ATGACCTCACTGCAGGACCTGGATTATCCACAACAGTTACTT
GAATATAAAGATGGATTCCTAGTCAGCAAGACAATGTTCACCGCGTGTGA
ACTGGGGATCTTCGATCTTCTACATAAGTCTGATGAAGCCCTGAGTGCTT
TAACAATCTCCAGCCACCTGGGGACCAGTGCCGACGGAACTGATAGACTT
CTGTCCGCCTGTGTTGGTCTAAAGTTGTTAAAGGTCGAAATGAAGAACAA
TGAGGCATTCTTCTCAAATACTGATGTGTCCGATGTTTACCTGGTGCAAT
TAAGTCCTAGAAGTTTGTATCATATGATGATGTATTACTCCCAAACGCTG
TATAAATGTTGGCATTTCTTACCCGATGCAATAAGAGAGGGAAAGAGCCA
ATACGAGAGGGCCTTTGGCGTTTCCTCTGGCGACATCTTTAAGGCTTTAT
ATAGGTCAGAGGAGGAAATGGTTACCTTTATGCACCATATGGATAGTGTT
TGGAACATCTGTGGAAAGGACATCATCGCGGCTTTTGACCTGTCTTCCTT
TAACGAGGTTTGTGACCTTGGAGGATGCAGCGGGGGACTAGCTAAACAAC
TTTTGAGTATATACCCGTCCTCCAGCGTGACAATTCTGGATCTACCCGAA
GTTGTCCAGACCGCTAAGAAGCATTTTATAACGGATGCAGATTGCAACAT
TGCGTTCTTACAAGGTAACTTTTTCAATGACCCAATTCCTGAGGCAGATT
TATATATAATGGCCCGTATTATCCACGACTGGACCCAAGAAAAGTGCTTA
CAGTTACTTAATAAGATCTACAAAAGCTGCAGACCCGGAGGCGGAGTACT
TTTGGTGGAAGTACTATTGAACGAAGATAGGAGCGGCCCTCTGACCAGTC
AGCTTTACAGTTTGAATATGCTTGTTCAGACTGAAGGTAGGGAAAGAAGC
CCGTGTGAGTATACGAAATTGTTGGCCCACTCCGGGTTCAGGGACATCCA
AGTAAAGGCGACGGGCAAGATTTATGATGCCATTCTAGGAAGGAAA

Seq. ID No: 131

>MUP1ATGTCAGAAGGCAGAACGTTCTTGAGTCAACTGAATGTGTTTAAT
AAAGAAAACTACCAGTTTTCAAGCTCAACAACGAAGAAAGAGGTATCCAA
TTCAACGGTGGACGCAGACAACGGAGCCTCAGATTTCGAGGCGGGACAGC
AATTTGCGACCGAATTGGATCAAGGAGAGAAACAACTTGGCATATTGAGC
TGTATCGGTTTGATCTGCAATAGAATGTTGGGTACTGGGGTATTCGCAGT
ATCTTCTACCATCTATACATTATGCGGTAGTGTAGGACTGGCTCTTATTA
TGTGGGCGGTCGGCGCAATAATCGCAATTAGCGGATTGTATGTCTACATG
GAGTTTGGAACAGCGATTCCCAAGAATGGCGGTGAGAAGAATTACCTAGA
GGCTATTTTCCGTAAACCCAAATTTTTCATCACATGTATGTACGCAGCGT
ATATCTTCTTTCTGGGTTGGGCAGCCGGTAACAGTATTAACACTGCTATC
ATGTTTTTGACTGCTGCGGACACGGAAGTCACCAAATGGAACCAGAGGGG
GATTGGAGTTGCTGTGGTGTTTTTCGCTTTCCTGATCAACAGTCTTAATG
TTAAAATTGGACTTTATCTGCAGAATATACTTGGAATTTTCAAAATCGGT
ATTGTACTTTTCATCTCCATAACTGGTTGGGTGGCGTTGGGAGGCGGGCT
GAAGGATGGATATCAATCTCATAATTTTAGAAATGCCTTTGAAGGAACGG
AGACCGCTACTGCCTATGGGATCGTCAACGCATTGTATTCTGTTATTTGG
TCTTTTGTTGGATACTCTAATGTCAATTACGCTTTAGGAGAGGTAAAGAA
CCCTGTAAGGACGTTAAAAATAGCCGGTCCCACGTCAATGGTGTTTTTGG
CTATCATCTATATATTTGTAAACATAGCTTACTTCGCAGTGGTACCCAAG
GATAAGCTAATAAGTAGTAAACTAATCCTTGCAGCCGATTTCTTCGATAT
TGTCTTTGGCGGTCAAGCTAAGCGTGCTGCAGCAGCGTTGGTTGGTTTGA
GTGCGTTGGGCAACGTCTTGAGTGTAATCTTTTCACAGGGCCGTATTATA
CAACAGCTAGGGAGGGAGGGCGTACTACCCTTTAGCAACTTCTTCGCAAG
TTCAAAACCCTTCAACTCTCCAATGGTCGGCCTTTTCCAGCACTTCATTG
TCTGCACAGTAACTATACTGGCCCCACCACCCGGCGACGCTTATCTGCTG
GTCCAAAATCTAATTAGTTACCCTATGAACATTATCAACTTCGCTATCTC
CGCAGGGTTGCTGTGGATCTATTGGCAGAGGAGGCAAGGTAAAATAGAGT
GGAACCCACCTATCAAAGCTGGGGTTTTTGTAACCGGCTTTTTCACCCTT
TCAAACTTATACTTGATCATAGCGCCCTATGTCCCTCCGAGTAATGGCGA
ATCTGTGTACAGTAGTATGCCCTATTGGATACATTGCGTCATAGCCTGGG
GCATCTTCTTTTTCGGGGGTGTATATTATGTGGTATGGGCTCAGTTGTTG
CCCCGTTGGGGGCATTACAAGCTCGTCTCAAAAGATGTTTTGGGTGAGGA
CGGTTTCTGGAGGGTGAAAATCGCAAAAGTATATGATGACACCATCGGTG
ACGTGGATACGCAGGAGGATGGTGTAATCGAAACCAATATAATTGAGCAC
TATAAGAGTGAACAGGAGAAGTCTCTA

Seq. ID No: 132

>NAT_1ATGGCTCCTATCGAAGAAGAGGAGCCGTTACCTGAGGAGCTAGT
CCTGCTTGAGAGGACTCTGGCCGACGGTTCAACGGAGCAGATCATATTCA
GCAGTGCTGGCGATGTGAACGTCTACGACCTACAGGCTCTTTGCGATAAA
GTTGGGTGGCCGAGGAGACCCTTGACCAAAATAGCAGCTAGTTTGAGGAA
CTCCTACTTAGTAGCTACGCTGCATAGCGTTACAATGCCTTCCAAAGCCG
AAGGCGAGGAAAGGAAACAGCTAATAGGCATGGCAAGAGCAACTTCCGAT
CATGCTTTTAACGCCACTATCTGGGACGTATTGGTTGATCCATCCTATCA
GGGCCAAGGTTTAGGTAAAGCTCTGATGGAAAAAGTAATAAGGACGTTGC
TGCAAAGGGATATCAGCAACATTACTTTGTTCGCAGATAACAAGGTCGTT
GATTTCTATAAAAACTTAGGGTTCGAAGCCGACCCCCAAGGTATTAAAGG
TATGTTTTGGTATCCGCGTTTT

Seq. ID No: 133

>NAT_2ATGAGTACACCGAGTGTTCACTGTCTAAAGCCCTCCCCCTTGCA
CCTTCCGTCAGGCATTCCTGGTTCTCCAGGTCGTCAGAGAAGACACACTC
TGCCCGCGAACGAATTTAGATGCCTTACGCCTGAAGACGCGGCTGGAGTT
TTCGAAATCGAACGTGAGGCTTTCATATCCGTGTCTGGTAACTGTCCCCT
GAACCTGGACGAAGTCCAACACTTTCTGACTCTGTGTCCCGAATTGTCAC
TTGGATGGTTTGTGGAAGGGCGTTTGGTAGCGTTCATCATAGGATCTCTA
TGGGATGAAGAGAGACTAACTCAAGAGAGTCTTGCACTTCATAGGCCGCG
TGGACATTCCGCCCATCTACATGCGTTGGCCGTACATAGGTCATTCCGTC
AGCAGGGGAAGGGCAGTGTTCTTCTGTGGAGGTATCTACATCACGTCGGG
GCACAACCAGCCGTGAGGCGTGCTGTGCTGATGTGCGAAGATGCACTGGT
CCCGTTTTACCAGAGATTCGGTTTCCACCCGGCTGGTCCTTGCGCTATTG
TCGTTGGGAGTTTAACGTTTACTGAGATGCACTGTTCTCTGAGGGGGCAT
GCTGCACTACGTCGTAATAGCGACAGG

Seq. ID No: 134

>NAT_3ATGACAAGTGATGTCGGCGCTGACGAACATGCTACAACAGAGGC
GGGCGGCGGGCGTTTACAGGCCGGCGGACATTCTTCTGCTGAAGAGGCTT
CCGAAAGATGCCCGCCTGCTGCTGCGCCGCCGAGTGGTATGAAAGGAGCG
GCTGATTGCGGGCCACAAGATTCATCAGCAAGAGATGACGTGTCTTTTAT
TCCCTATAAAGATGAAACTGACATGCCAGGAATCGTGGAGCTTATCGAGA
AAGATTTAAGTGAACCTTATAGCATATTTACTTACCGTTATTTCATAAAT
AACTGGCCAGAGCTTTGTTTCTTAACTATGCGTGGTGATAGTTGCGTCGG
CGCAATCGTGTGCAAATTAGATGTCCATAGGTGTAGGAACACGAATAGAG
GCTACATAGCGATGTTGGCAGTTGAAAAGGGATTACGTGGTAAGGGAATA
GGAAGCACTCTTGTCCGTCTGTGCCTAGATAAAATGAGGGAAATGGGAGC
GGACGAATGTGTACTTGAGACGGAGGTCACTAACAAGGGCGCACTGGGGT
TGTACCGTAATATGGGTTTCGTAAAGGAAAAGAGGTTACACAAATATTAC
CTGAATGGCAATGATGCCTTTAGATTGAAATTCTTGTTCAAGCTGCCCGA
AGGCTTCGATAGAGGAGAGGGTTGTCTAGGTCCTTTATGTGAGGTACCTC
CAGTGACGACT

Seq. ID No: 135

>NAT_4ATGGTGTCCATACGTCCTGCCACAGTGGACGACTTACTTGCGAT
GCAAGCATGTAATCTATGCTGCCTGCCAGAGAATTATCAAATGAAATATT
ACTTCTATCACATGCTGAGTTGGCCCCAGTTGCTTTATGTTGCGGAAGAC
TACGGAAAGAAGATTGTAGGCTACGTGTTAGCGAAAATGGAAGAAGACAG
TAGCGAGGTCCACGGTCATATCACATCCCTGGCCGTGCTTAGAAGTCATA
GGAAACTTGGTTTAGCGTCTAAGCTGATGCGTGCGGCGATGGCTGCGATG
GAAGAAACATTCGGAGCAGAACATGTCTCCCTGCACGTGAGAGTTACTAA
CCGTGCCGCCTTCACATTGTATTCAGAAACGCTAGGGTTCGAAATTAACG
ACGTTGAACATAAATATTATGCCGATAAGGAAGATGCTTATGATATGCGT
AAGATGTTTGAAACAGGGCTGAAGAAGCAAGAGGCCGGTAAGCAAAAGAA
AAAGGAAAAGGAGAAGGAGAAAGAAAAGGAGAAGGAAAAAGAAAAGGAGA
AAGAAAAGGAAAAGGAGAAAGGTAAGGACAGCCAGCCGGTGGAACAGCAG
GGTGGTGCTGCCGGTGCTGACAAGGAAGCGCAACGTTCCAAAAGAGCGCG
TTCAGGCGACAGGAAAAGGAACGGCCGTCCCCGTAGGAGAAGTGGATCTG
GG

Seq. ID No: 136

>NAT_5ATGCTTCCACGTCCGCCGGTGGGCGCGGCCAAGGAGGGGCACCT
TACGTTATTTTACCGTGAGTTAAGATGGCTTTGCCCTGGAACTCGTTTTT
ATTTCGTAGTAAGGGACCCCGCCGAAAACGTAAGATCAATAGCAGACAGA
CTGGCCTTAGGACCAGAAGGCCTTAGGAGACCTCCAAGGATAGTAGCTAG
AGCAGACTTAGGATGGCGTGAGGTACTAAACATGTCATACGCAGGAGTGA
GGGAAGAATCAGCGTTGGGGACTCTGGTTGGCAGGTGGAACTTAATGGCG
AGATTGTACCTAGACGCGCCGAAGGGCGCGATGGCCCTAGTGCGTTATGA
AGATTTGGTTGCGGAAGCAACTTGGGAAGCTGAAGTCAGACGTGTAGCGG
CAGCCGAAACCTTAGACCTAAGGGAGAGAGTCCTTTGGCCTGGTCGTCCT
GACCTTTGTACTTTGCCGGGGGATGAATCAGCTCTGCATTTCGGGGCAGT
AGCTGCTGGCAAAGTTCTAGGCGTCATTTCTGTGTTTCTTTCTCCGGAAC
CAGGTGGTAGGGCCCAATTCAGGAAATTTGCTGTAGACCCTGAGGTCCAA
GGGAGAGGGCTGGGTCGTAGACTACTGGAGCAGGCGGTTGCAGCAGCTAG
AGAAGCAGGGGCCGGTAGCTTATTTTGTCACGCAAGAGCAGACCAACAGG
GGTTCTACGAGAGGCGTGGATTGCATGTGGTCGGTGAACCATTCGAGAAA
TACGGTGGCAAGCCCTACGTCGAGATGGAAGTGCCGTTCCAA

Seq. ID No: 137

>NAT_6ATGAAAGGCTCAAGAATAGAGCTAGGTGACGTCACACCACATAA
TATCAAACAGTTAAAAAGGCTAAATCAGGTCATCTTCCCCGTTAGTTACA
ACGATAAGTTCTACAAAGATGTGTTGGAAGTAGGGGAGCTGGCGAAACTA
GCGTATTTTAACGACATAGCAGTAGGCGCTGTGTGTTGCAGAGTCGATCA
TTCCCAAAATCAGAAGAGGCTATACATTATGACTCTAGGCTGCCTTGCGC
CATACAGGCGTCTGGGTATAGGAACTAAGATGCTAAACCATGTTTTGAAC
ATCTGCGAAAAGGATGGTACTTTCGATAACATATATTTGCATGTCCAAAT
AAGCAATGAGTCCGCTATTGACTTTTATCGTAAGTTTGGTTTCGAGATTA
TCGAGACGAAGAAGAATTACTATAAACGTATAGAGCCGGCGGACGCGCAT
GTCTTACAGAAAAATCTAAAAGTAAGTTCCCCTGCTCCCAATGCCGACGT
CCAAAAATCCGAAAAT

Seq. ID No: 138

>NAT_7ATGTCTACTCCGTCCATTCATTGCCTGAAACCTTCACCGTTACA
CCTACCATCTGGCATTCCCGGTAGTCCCGGTAGACAAAGGAGACACACGC
TACCGGCCAACGAATTTAGATGCTTAACCCCGGAAGATGCGGCTGGAGTG
TTTGAGATAGAAAGAGAGGCTTTTATATCCGTATCAGGGAACTGCCCATT
GAACCTAGATGAGGTAAGACACTTCTTGACCCTTTGTCCCGAGTTGAGTT
TAGGGTGGTTTGTAGAAGGTAGGCTAGTGGCGTTCATTATAGGGTCACTG
TGGGATGAGGAAAGACTTACCCAGGAGTCTCTGACATTGCACCGTCCCGG
CGGGCGTACAGCGCACCTACATGCCTTGGCGGTCCATCATAGCTTCAGGC
AACAGGGCAAGGGCTCTGTCCTACTATGGAGATATTTGCAGCATGCTGGT
GGCCAGCCAGCGGTTAGAAGGGCTGTACTGATGTGTGAGGATGCATTGGT
GCCGTTCTATCAAAGATTCGGATTCCATCCGGCAGGTCCTTGCGCCGTTG
TTGTGGGTAGCCTAACTTTCACTGAAATGCATTGTTCTTTGAGGGGGCAT
GCTGCCCTAAGACGTAATTCTGACCGT

Seq. ID No: 139

>NAT_8ATGTCTTCTGGTGGCGTTATCGTAGACTTACATAGGAACTCTAC
CAATTGGGCGAAAGTAGTAGACGACATAGTTAAGTTAGAGAGGAAGATTT
TCCCCAAACACGAAAGTCTTGCAAGGTCTTTCGATGAAGAATTAGGTAAG
AAGAATACAGGACTAATATATATGGAAGTAGACGGTGAGGTTGTTGGTTA
TGCTATGTACTCTTGGCCTAGCTCAATGTACGCTTGTGTAACTAAGTTAG
CTGTCAAGGAGAACTGTCGTCGTCAGGGTCATGGGGAAGCCCTGCTAAAA
GCTGCAATTAAGAAATGTAGGACCAGAAACGTTCATCGTATCAGTTTACA
CGTGGACCCTCTAAGAAACCCGGCTATTTCTTTATACAAAAAGTTCGGCT
TTCAGGTGGATAATCTTATTGATGGGTACTACAGCTCCGATAGGAATGCC
TACCGTATGTATCTGGACTTCGACGCTGAT

Seq. ID No: 140

>NAT_9ATGGATGAACGTGTGGTGGTTGAGTTAAAGAAGTCTTTAGCTGA
CTACCCGAAAGTCCTAGAGGAACTTGTGAGAATAGAGAAGAAGGTGTTTC
CTAAGCATGAGTCCTTGAGTAGATCCTTCGACGAGGAGCTAGGTAAAAAG
AATAGTGGGCTTCTGTATATTTGCTCAAATGGCGAAGTTGCCGGTTATGT
GATGTATAGCTGGCCTAGTGCATTGCTGGCGGTAATTACTAAATTGGCTG
TAAAAGAAAAGTATCGTCGTCAAGGCTATGGCGAAGCCTTATTGCGTGCA
GCGATTCAAAAATGTAAGACTAGGAACATCCAAAGGATCTCATTGCACGT
GGACCCTTCTAGGACACCTGCAGCGAACTTATACAAGAAACTTGGCTTCC
GTATTGACTCTCTAGTTGAAAAGTATTACGCGGCAGACCGTGACGCCTAT
AGAATGTACCTTGACTTCGACGCCGACGTC

Seq. ID No: 141

>NAT_10ATGATGGAGGGTGCCCAAGAGGACGAAGAAACGGAAGAAAAAG
CTGAGTTCGATGCCAGCGAGATTGAGTACGTGAGTTACGGAGGGGAGCAC
CACCTTCCTCTAATCATGTGTCTAGTTGACCATGAACTGTCAGAACCGTA
TAGCATCTTTACCTATAGATACTTCGTTTATTTGTGGCCGCAACTTTGCT
TCCTGGCCTTTCACAAAGGAAGGTGCGTAGGAACCGTGGTTTGTAAGATG
GGTGACCACAGGCATACCTTCAGGGGCTACATTGCTATGTTAGTTGTAAT
CAAACCTTACCGTGGAAGGGGCATAGCTACGGAGTTGGTTACTCGTGCCA
TCAAGGTAATGATGGAGTCAGGGTGCGATGAAGTTACATTGGAAGCTGAA
GTGACGAACAACGGGGCGCTCGCACTGTATGGCAGATTAGGATTCATAAG
GGCCAAGAGGCTTTTCAGGTATTACCTGAATGGTGTCGATGCGTTCAGAC
TGAAATTGCTGTTCCCCCGTAGTGAAATGCACCCCAGTTTACACCTTCTA
GCGGACCAAGACGGACATGATGACCAGATTGCAATGGAGGGGGAAGCT

Seq. ID No: 142

>NAT_11ATGAAGCAAGTTGGGATTAGCCTAGATGCAGTTAGAGAAAAGA
ATCTAATGCAGTTGAAAAAGCTAAATGTGGTACTATTTCCTGTGCGTTAT
AACGATAAGTATTACGCCGATGCTTTAGCGTCAGGTGAGTTTACAAAACT
GGCGTACTATTCCGATATTTGTGTTGGCGCTATAGCATGTAGGCTTGAGA
AGAAAGACCCCGGTGCCGTACGTGTGTACATAATGACACTTGGCGTTTTA
GCTCCCTATAGGGGTCTTGGTATAGGAACTGAGCTTCTTAATCACGTACT
AGAGCAGTGCTCCAAGCAAAACATTAGTGAAATCTATCTTCACGTCCAGA
CAAACAACGATGATGCGATAAACTTTTACAAGAAATTTGGTTTTGAGGTA
ACGGAGACGATTCAGAATTATTATACCAATATAACACCTCCCGATTGTTA
TGTCGTAAGTAAAAGGCTGGAAGCTCAACCAAAGAAG

Seq. ID No: 143

>NAT_12ATGAATATACGTGTCGCAAAGGTTGAGGACCTTATGGGAATGC
AAGCATGTAACTTGCAAAATTTACCCGAAAATTATATGATGAAATTTTGG
ATGTATCACAGCATGACCTGGCCACAAATTTCATTTGTCGCCGAAGATCA
TAAGGGGCGTATTGTGGGGTACGTACTAGCAAAGATAGAAGATCCGTCTG
AAGAGGGTACGACTGAAGAGATTCATGGCCATGTTAATTCAATATCCGTG
CTTAGGTCCTACAGGCGTCTAGGCTTAGCGAAGAAACTGATGCTTCTAAG
CCAAGAGGCAATGTCTAGTATTTACAAGGCTTCCTACGTCTCTCTTCATG
TCCGTAAGTCAAATAAGGCGGCCATTGCCCTGTATAAGGACACCCTTGGC
TTTGAAGTGGCTAAGGTGGAGAAGAAATACTACGGGGACGGTGAAGATGC
GTTATCAATGAGACTAAGTCTGAAGAACCCT

Seq. ID No: 144

>NAT_13ATGAGTGACTTCCAAGTTGCTCCACTTACTGCAAGGGAACTTG
CTAGGGTCAGGGATTTGCACGCAAAACTGCTGCCGGTGCAGTATCCAGTA
AGTTTCTTTATCCACCTATTAGTGATACCCAGTAGGGCCTGTTACGTCGC
CTATAGTCATGGGAGTCCAGTGGGCTTCATTAGTGCTGCACTACACAATC
CTACCAGATGCTTCATATCTGGGGATTCTGAAGTGAGCCCCCGTTTGGAG
ATTTTAACCCTTGGGGTGCTACCCGCTTTTCAACACAGAGGTTTGGCCCG
TAGACTAATCATGAGTCTTGTAAACGCCTTCAAGCAGGACCCGGCTACCC
CTATTTTGATATACGCAAACGTATCAACCACAAACACTCGTGCGTTACAA
TTCTATGAAAGAATGGGCATCTTGGTTTCTTCCGACATAATAACTAATTT
GTACAGAACTTTATCTTACGGGTCTCGTGATGCTTACCTAGTGGTAGGAG
CCCTT

Seq. ID No: 145

>NAT_14ATGTTGTCTATACATCCTCTGAAACCAGAGGCGCTGCACCTTC
CTCTGGGCACATCAGAGTTCCTTGGGTGTCAACGTCGTCACACATTACCC
GCGAGCGAGTTTCGTTGTCTAACGCCGGAAGATGCCACTTCAGCATTCGA
AATAGAGAGAGAAGCATTCATAAGCGTCAGCGGCACATGCCCGCTGCACT
TAGATGAAATTCGTCATTTCTTGACCTTATGTCCCGAACTTAGTCTTGGA
TGGTTCGAAGAAGGATGTCTGGTGGCTTTCATTATCGGATCTCTATGGGA
CAAGGAAAGACTAACACAGGAATCTCTAACTCTTCACAGGCCCGGTGGGA
GAACTGCACATCTTCACGTGTTAGCTGTACACAGAACTTTTAGACAGCAA
GGAAAGGGCTCAGTGTTACTTTGGAGATACTTACATCATCTAGGGTCTCA
ACCTGCGGTTCGTAGAGCGGTCTTGATGTGCGAGAATGCGCTAGTGCCAT
TCTATGAGAAGTTTGGTTTCCAGGCAATGGGACCCTGTGCTATCACTATG
GGTAGTTTAACCTTTACAGAGTTACAATGCTCACTTCGTTGTCATACATT
TCTAAGGAGAAACAGTGGATGT

Seq. ID No: 146

>NAT_15ATGGCTGATGCCCCTTCCGGCCCCTCTGTCCTTTCTCACTATC
CTGGGGCCGGTTTAGCCTTGCCACCCGGTGACGAGCAAGAAGATGGTGAA
GAAGAGGAAGAGGGGCGTTACGAACCAAGACGTGGCCACCATCACCATCG
TCGTCACCACCAACAACAACAGTTGAATGGGCTAATATCACCTGATCTGA
GGCACATTAAAGCCCTTAAAAGTAAATTACCACCTCCTCCGCACGATGAA
AGGACCGGCGCACCGAATGGACTAGAGAGACTGCAAGACCTTGAGGAAGA
GGAAGCCGTCTTGGCTTCTAGGATGGGTGCCTGTTCCCTTCACCCTGGTG
ATGGCAGCATAAGGTACGTACGTTACGAGTCAGAATTACAGATGCCAGAC
ATAATGCGTTTGATTACAAAAGATTTGAGTGAACCTTATAGCATCTACAC
TTATAGATATTTCATACACAACTGGCCCCAACTGTGTTTTCTAGCTATGG
TAGAAGAGGAATGTGTGGGAGCCATAGTATGCAAATTAGACATGCACAAG
AAAATGTTTAGACGTGGTTATATCGCAATGTTGGCCGTAGACTCAAAATA
TAGAAGGAAGGGTATTGGAACGAACCTAGTGAAGAAGGCAATTTATGCTA
TGGTGGAAGGCGACTGCGATGAGGTCGTGTTAGAAACGGAGATAACAAAT
AAGTCAGCCTTAAAGCTGTACGAGAACCTAGGATTTGTCAGAGACAAAAG
GTTGTTTAGATACTACCTAAACGGTGTGGATGCGCTTAGGCTTAAACTAT
GGTTACGT

Seq. ID No: 147

>NAT_16ATGGATGCCGCAATGCCAACAGAAATTTCTTTTCGTCAGCCAA
CTCCTGACGACGCGGCCAGATGTTTCGAAATTGAAACTAGCGCATACGAG
GGTGATGAGGCTGCTACGCTTGAGAAGATCGCAACAAGGATCGCTTTATA
TCCGGAGGGCTTCGTCATCCTGGAGGCGGACGGAAAAATCGCAGGGTTCA
TAAATTCCGGTTGTGCGTTCGAGGTGGTGATGAGTGATGAAGAATTTAAG
GAGTTGGTCGGTCACGACCCTGCTGCACCTAATGCTGTCATCATGTCCGT
AGTGGTAGATCCTGCAGAGCAAGGCAAGGGCTATTCTAAGCTACTAATGC
AACATTTTATAGCCAGGATGAAAGCTATGGACAAAAAGACAATCCATTTA
ATGTGTAAAGAGGCCCACGTCCCGCTATACGCTAGAATGGGCTACCGTTA
CACACGTCCATCTGCTTCCGATCATGGAGGCATGGCTTGGCACGAGATGG
TGATGGAATTG

Seq. ID No: 148

>NAT_1ATGGAAGGCTTACACAGCGAATGGGAAGTAGGCGCAGAGTTAAA
GGCTCTGGGTGCAGTTCCGAAACCTTTCATCGGAAGTCATGTATCAGGTA
AACTGATTCAGCGTTTAAAGCAAGACCTAAGGCAGAGCTGGGATCGTGGG
CAATCCCAGGCCAGACCGACTTGCACCTTGCCTCAACCTTTGCCTGCGCC
TCTAGGCAGTTCCGTGCCGAGCGCGTCCGCCCAAACGCAAGTCAGTAGGC
TTGTTCCAGTAGCCCCGCCACAACCAGATCCTGCCATGAGTGTCTTAAAC
GCAGTACCCTTTATGAGACCCATCCACCTTAGAAGCCCGAGACAGCAGCG
TAGGCACACATTGCCGGCCTCCGAGTTTCGTTGCTTATCTCCGGAAGATG
CTGTTAGCGTGTTTGAAATTGAACGTGAGGCTTTCATTTCAGTGAGTGGG
GATTGTCCTCTGCACCTAAACGAAGTAAGACACTTTCTAACCCTGTGCCC
GGAGTTAAGCCTTGGATGGTTCGAGGAAGGGAGGCTAGTCGCTTTCATCA
TTGGTAGTCTGTGGAACCAAGACCGTCTTTCTCAAGATGCATTAACTTTG
CATAAGGCCGAGGGTAGTAGTGTCCATATACACGTTCTTGCAGTTCACAG
AACATTTAGACAGCAGGGTAAGGGGAGCATCTTACTATGGCGTTACCTAC
AGTACCTAAGATGTTTGCCTTTTGCCAGAAGAGCGGTACTGATGTGTGAG
GACTTTCTGGTTCCATTCTACAGCAAGTGTGGGTTCAAGGCAGTAGGTCC
GTGTGATATAACCGTTGGTCCATTGACATTCATTGAGATGCAGTGTCCAG
TACAGGGCCACGCATTTATGCGTAGGAATAGCGGATGC

Seq. ID No: 149

>PSIK_1ATGCAGGCTAACAGACCTATATCCGACCAAGATCAAGACCAGT
TTAAATTGAATCTTACCACTGCAGATGGAACTAGGTCTTATCTGGAGAAG
CATTTGAGCCTGAACGTGGAGGCGGTTGAACGTTTATCCGGTGGGTTCAT
AAACTTTGTTTGGAGGGCTAAGTTAGGAACCCCCTACGAAGGTCAGAACT
CAATTGTTGTAAAGCATGCACCCCCTTTCACAGCAATGGACTCATCTCTT
AATGTCGCGGTAGAGAGGTTGAAATTCGAGTATGACTCTCTTAAAATGAT
AGGTAGCGAGCCAAGTATTGCCGGCGAGGATGCCCTTATATCAGTCCCCT
CTGTGTACCACCATGATAACATTAAGCATGTGTTGATCATGCAGGATGTT
GGAACCATGTCAACCCTGAGAGATTTCATGGGAGCATCCCCTCCTCCGCC
AACTGACATGGCTGCACTGATTGGGTGCCAACTAGCCACGTTCATTGCAG
GACTGCACAACTGGGGCAGAAACAACGAATCTGCTCGTGCTGGTCTATCA
GCAAATGCTTATGGGAGAACGGTCATGGACCTATGTGGATACCAGACAGT
GGTGCCTAACGCTACGGCAAGTGGGATTCTGGACCCGCTGTTATCAACTG
CAATGGCAGCTTTAGCCGAGCGTGATAAGACCTCCGAAGAAACAGCCATC
ATGGGAGACTTCTGGGCCCTTAATGTACTTGTTGATATTGACATGTCAGC
ATCAGGAGAAAAAGCCCTTAAAAACATATGGATCGTCGATTGGGAAGCAT
GTAGATATGGCTCACCAGCGGTGGACGTAGCGACCTTCGCAGGAGACTGT
TATTTAATTTCACGTATTCACAATGAAACCGCAACCGACGCGATGCGTCG
TAACTTTCTTGGCACGTATGTCGCATTAGCTAAGGTGGACCCTATGGAAG
TTGTGATCGGAATGGGCACGATGTGGATTATGTGGACTAAGTACCAAGAG
GATATAGGGGAAGCCGAAAAGAGGGAGAGGGTCGCAAAAGGCGTCGAATA
CATCCACAAAGGATGGGAACGTTCCAGAGAGTGGCTGCCTGTCTCATTAG
CGCAGGAGTTGATCGCA

Seq. ID No: 150

>PSIK2ATGGACCTGACCACCGGAGACGGCGTTCGTGTATACTTGACGGC
TCACATGACGCTAAAGGTTGAGAGCACAGAAAGATTGTCCGGTGGTTACT
GTAACTTCGTATGGAGGGCTAAGTTAAAGACACCTTATGAGGGACAAAAT
AGTGTAATCGTGAAATACGCGGCCCCCTTCACTTCCTGGGATCAGACTAT
TGAATTGGGCGTTGAAAGACTAGCATTTGAGTGCATGTCACTAAAAATGA
TTACGTCCGAAACTCCACTGCTTGAGGAAAATGGGTTAGTTGCAGTACCT
ACAGTATATCACTACGATTCCACAGCCAATGTGTTGGTCATGCAAGATAT
TGGTTCCATTGCGACTTTGCATGGGTTTTTGCGTTCCAATACTCCCCCAA
CGGTGCCGATGGCCGCACTAATCGGGGCAAAGTTGGCCGCATTCATTGCG
GGGGTGCACAATTGGGGAAGGAACAACCTACCAGCCCACACCAGATTAAG
CGCCAATACTGTCGGCCGTACTGCAATGAAGAAATTGTGTTACGAAACGA
TCGTTCCAAAGGCGGCAAAGTCCGGGGTGGTCGATCCCCTGCTTCCGATG
GTAGTGGCCGCTCTATCTGAAGAGGCGATGACGAATGACGAAACCTTGGT
AATGGGAGACTTTTGGACTGCGAACGTACTTATTGACGTTCAAGAGTCAC
ATACCGGGGAGCAGGTCCTGAAGAAACTGTGGGTGATTGACTGGGAATCA
TGCCGTTATGGTAACCCCGCGACCGACATCGCGAGCTTCGCGGGAGACTC
TTACCTTGTGTCCCGTTTCCAGGACCACGGTTTAGGTGAAGCCTTGAGAC
ACAGTTTTCTTGAAACCTATGCGGCGTTAGCAAAAGTCGATCCACTACGT
GTTGCTTTGGGTCTGGGCGCGCACTGGATAATGTGGACCGATGATCTAGG
GCAAGGTGGAGAAGCGGAAACCCGTGAATGCGTCGATAAAGGCTTAGAGT
ATATTCAACGTGCGTGGGATCAAAGCGCCGAGTGGGTCTCATTGTCTTTG
GCAAAAGAGTTGGTAGTCTTG

Seq. ID No: 151

>PSIK3ATGGCAAACGAGAACCCTGACTTACTTACAGTCGCGGGCGTTCT
AAGGTTTCTAGCTCCAACTCCTTTCGCTAGTGATGAAGTTCATCCTCTTT
CCGGGGGCAATTGTAACTTTGTGTATAGGATTCACTTGAGGACACCGTAC
AATAATATATCTACTCTGGTCTTAAAGCACGCGGAGCCTTACGTCGCTGC
GTCCGCTCACAGGATGCCCCTTGCCGTGGAGCGTCAGAACACGGAAGTCA
CTGCCATGAACGCAGTCAAGGCAATTCTATCATCCGATGCGGTAGTTATA
GTTCCTACAATCCATCATTTCGACGATGTCGCACATGTAGTTATCATGGA
TGATTGTGGAGTCGGAGCCGTCACCCTAAAGCAATTGATGTTGAAAAACC
CACCTCCTGTTTCCGTGGCAAAAGCGTTGGGGGCAGGTCTAGGAGAGTTT
TTAAGTCGTCTACATGTGTGGGGGAGGGACCCGCAGACGTCTAATCATGT
CAGTTTCGATCAAAACCAACAGGGAAGGACAATAAGCGGCTATGTTACAT
ATGGTAGGTTAGTCAGCACCCTTACTGGTAAAGATAATATCCCGGCGTTA
TCCGACCCTCCGCTAGACATAGCGCAAAGTAAATTGGATACGATCAGTGC
ACTGTCTAGTGAGAAAATCCATGCCATAAACACTTCACATCAGACCCTGA
CTATGGGTGACTTCTGGCCTGGTAATATTATGGTTAGGCTGAATCCTGCG
GGTGATAGTTTGGAAAGGGCTTATGTGTTGGATTGGGAGGTTGCTAAGCC
TGGCGTGGCCGGTTTAGATATCGGCCAGTTCTGCGCTGAAATGCACAGTC
TAAGAAGGTTCAGTCCTGCCTGTGATGCTTCAGCTACCACCGTTCTGGAT
GCTTTTCTGAAGACCTACAGGGACGCAGCCGGAGTCGATGTAGGGGTAGC
CAAAGATGCGATGGTCCATGTCGGAGCGCATTTAGTAGCGTGGACGCCGA
GAGTGCCGTGGGGGAGTAAAGAAAGAACTCGTGAAGTGGTTGAAGAAGGC
GTTGGATATTTAGTTGAGGGATACGCAGCGACACAAGAATGGCTACGTGG
CTCTCTTGTGGGTAGGCTAGTA

Seq. ID No: 152

>PSIK_4ATGGAAATCGAATGGTGTGACCTAGACCTAACTTCAGAAAGTA
GACCTCCTACCCATAAATATACTTATTTCGCCACTGCCCTTATGCCCTTT
GATTTAACTACGAGGGATGGCGTCCGTATGTACTTGACCGCATACCTTGC
TTTAGACGTGATGTCTGTAGAAAGGTTGTCTGGCGGGTATTGCAATTTCT
CCTGGCGTGCGAAGTTGGAAAGCCCTTACGAAGGCCAAATCTCAATCGTC
GTAAAACACGCGGCGCCATTCACCTCTTGGGATAGAAACACAGAACTTGG
TGTTGAACGTCTTGCTTTCGAATACAAGGCATTAAAGATATTAAACAGTG
AACCGTCAGTCATCGCCAAGAACTCACTGGTCGCTGTACCGGCTGTTTAC
CACTACGACCCGACCGCCAACGCATTGATCATGCAAGACGTAGGTTCAAT
ACCAACCTTGCATGCATTACTAAGGAATAATGCATTACCACCAGTCCCAA
TGGCGGAGAAGATCAGTAATGAATTGGCGGCGTTTATTGCAGGCATTCAT
AATTGGGGAAGAAACAACCAAGAAGCAAGAGCTAATTTGAGTCAGAATTT
GGTTGGCAGGACAGCGATAAGGAAGTTGTGTTACGAAACATTGGTGCCAA
AAGCGGAGAAGTCTGGTGTTGATGACCCTCTATTGCAACAAGTTGCTGCG
GCGTTATCCGAGGAGGTCATGAACTCCGAGGAAACGCTGGTGATGGGTGA
TTTCTGGACTGCAAATGTGATGGTTGACATACAAGAAACCGGGGCAGGAG
TAAGGAGCCTAAGAAAGATATGGGTTATAGACTGGGAGGGCTGTAGGTAC
GGCTCACCTGCGGCAGACATAGCTAGTTTCGCTGGGGACTCTTACCTTGT
CGCAAGGTTTCACCATCACGATCTTGGAGAGACTTTAAGGCATAGCTTCC
TAGAGACCTATGCAGGTCTAGCAAAAGTAGATCCTTTCAGGGTCGCGTTA
GGTTCTGGTGCACACTGGATAATGTGGACTGATGATCTTTCTGAACAAGA
AGAAGGTGAAATCCGTGAATGCGTAGACAAAGGGGTGGAATACATTCATC
GTGCATGGGAGCAATCCACTAAATGGATTAGCTTGTCACTAGCCAAGGAA
TTAGTCACA

Seq. ID No: 153

>PSIM_1ATGCATTTCAGCTACGATTTTACTATGTGTAACCCGCCGTTTT
ATGGTGACTACGCTGAGCTAACGAGGCTAAGAGAATCCAAGTTGAAGGGA
CCGTTCGGGGGCGCTCATGAGGGAGTAAGTACTGAACTATTTACTGCTGG
AGGCGAAATACATGAGTTCATAGCAAGTGAACTGAACCAGGGCAAAACGC
TGCGTTGGGTGGTAGGATGGACGTTCCATAAGGATTTATTCGATAAAAAG
GTTATCGAGCCATTACATATTTTAATAAAATTGAAGCCCCTGAGCTGTAA
CACCTTGGAGCTACTAGATTCCGACCACGAACTTGCTACGCCCTACAGCA
AGAAGCGTTGTCTTAGGCGTATCTCCTTTACGCCCAAGGGGAGGCCACCT
ACGAAACAAACTATAAGTGTAATGCACCCCAATGAGGCTTCGTGGGCATC
TCTGCTAAATCACCTTCAAGCATTGGACATTTCCGTGACCGTAGGCAACC
ACTTTCTAACAGCGGAAGTGAAAGACCCAACGTGGACAAGAGCGTGGAGA
CGTAGCTCTAAGGTAACTTCTAAGATCACCCCTTTTAGTTTCTCTGGACA
GTTTTCCGATCCACCAGAGAGGTTGTTAGTATTACAGCTTCTTGTCGATG
AGAGTCAGACCAGCGAAGACATTCTACTGTCATTCCAGAGCTTG

Seq. ID No: 154

>PSIM_2ATGCACCCTCGTAATCCTTACAGAACCCCTCCGGATTACGCAG
CTTTGGCGAGGAGCTTCCCGGAACTGAAGCCTTACGTAAGTAGGAACGCC
AACGGCACAGTTTCCGTCGATTATCAAGATGAAGCCGCCCTAAGGTGCCT
AACCAGAGCCCTACTGTATAGAGATTTTGGACTATCCGTGGATCTTCCCA
AGGATAGGTTGTGTCCCACCGTTCCAAACAGGCTAAACTATATTCTTTGG
ATTGAGGATATTTTGAACGTCTCATCTTTAAGCAGACTACAAAGCAACTC
TGAGGCAACTGTGAGAGGTCTTGACATAGGAACCGGCGCCAGCGCGATCT
ACCCGTTGCTAGGCTGCAGGGTTAGTCCAAGATGGGATTTCTATGCCACG
GACATAGATGCCCAGAGCCTTGCCCATGCCAGGGCCAACATAACCCGTAA
TGGGCTACAGGGAAGGATTAACCTGGTTGCTGCGGACCCCAAGGGTTCTA
TATTTGGACCATTGGAGAGCAAACACGATACGACTTTTGAGTTCACCATG
TGCAATCCCCCATTCTATTCATCAGAGGAGGACATCGCCCAGAGCGCAGC
CGTTAAGAAACTTGCTTCTAACGCCGTCTGTACAGGGGCAGCAGTCGAAA
TGATCACTCCTGGAGGTGAGGCTGCGTTTGTGGTTAGGATGGTAAGAGAG
AGCCTTGCTCTAAAAATGAGGTGTCAATGGTACACGTCCATGCTAGGAAA
AATGAGTAGTCTGACAGAAATAGTGGGGCTGTTAAGGGAAAATAGTATTG
ATAACTACGCTATCACGGAATTTGTCCAAGGGAAAACAAGGCGTTGGGCG
ATTGCATGGTCATTCGGCCACGTAAGGCTACTTGATAGTCTGGCGAGACT
TTCAGGTGGGCCACTTCAGTCATTAATGCCAACTAGGAACACATGTCGTA
GGTCTTTCGCCGTTCCTCGTATGGTCTTACATAAGCATCTAATTTTGGTG
CTTGACGGCATAGAGGGAACATCCCAGACTCCCATGTCAATCCCAGTAGG
CGCTGGGGACGCCGACGGGTTGTACGGGCTACAGATCTCAGCTAGTAGAG
ACACTTGGTCCAGAGCGGCGAGGAGAAAAAGGCAACACGGTGCAATGGAT
ATATCATTAGATAATGATGAAGTAGGGATGAAGTGCCTGATAAAGGTGTT
ATCCGTAGAGGAAGCAAGAGAAGGAGCAGAGGCCGTAGTGCTAGAGTGTA
CCTGGGTGTATGGGCATGAAAGGGCGTTATTTGAGAGTTTTTGGGGCCAC
GTATGCCGTAAGATTGGGGAAGCAAACGGG

Seq. ID No: 155

>PSIM_3ATGCACGCGAGAAGCATATTCAACCCTAATTCAGCTCAGTTTC
AGGCACGTCTGACATTTTCTGAGTTGTCTAACGAGTTTCCCAAGTTGAAA
CCATTCTTGAAGTATAAGAGGTCCAGGAAGCAGAATGAAGCGGACCCTTT
ATCATCCCAGTGTACGTTTATTGATTTCAAAGATCCAGTCGCTACGCGTG
CCTATAATGAGGTGTTACTAAAGAAGTACTTTGACCTTAGCTTGGAATTT
CTGCCTGGCTCATTATGTCCGGCGGTTCCCAATAGATTAAATTACACCTT
GTGGCTAGAGGACGTCTTGAACGTCTTCCCAGGGAGTATGGGGGCAAATA
ACCAACGTGATGAGCTAAGAGGACTGGACATAGGAACTGGTTCATCATGC
ATATATCCCCTACTTATATGTAGGACGCACCCGAACTGGAGGATGGCGGG
GTCAGACATCAATCCCTCTTCAATAGAGATCGCCAAGAAGAACGTGCAGG
AGAATAGGCTGCTGGATCGTATACAGCTTTTCTTAACAACCGATAAGAGG
GATAGCGTCCTAGAGGGGCAAATATTTCAGACCCACTTGTTCTTCAATTC
AAAGAAATGCCTGCAAGATGAAAAACCTGCCAGATTCTGCTATGACTTCA
CGATGTGCAATCCACCATTTTATTCCGACGTTGAAGACCTTAATAACAGT
AGACAAGCGAAAACCACGACCATATTAGGTGGTGGACATGAAGGGGTGTC
GTCAGAATTGTTCACTACAGGTGGAGAATTACTGTTCTTATCGCAGATGG
TGGAGGAAAGTTTCTTGTATAAAGACAAGGTGGGGCTTGTTAGTTCATAT
TTCTTTGTGCTTAAATGCATAGTGGAGATTCTTCGTCTTCTGGGACAGCA
TAAGATACAGGAAACTATTGCGTCTAAGCTAATACAAGGCAAAACCATAA
GATGGGTAATAGGATGGACGTTCCACAAAGACCTTTTTGATCTGAAGCAT
CCCTCCTGTAACACTCTGAAGGTGCAGGATATACATTGCACGAACGAAGA
AGCCCCTGCGTCTAAGAAAATATGTCTAGGGAAAATTCCCCTTAACTCTA
AGGAACACATTCCGTCTAAGCAGCTTATATCTTCATCCCAGTCAAGCGAC
ATTGGCTGGACCAGACTGAAAAGCAGACTTGGGGACCTTAGAATCGAGTT
CTCTCTTGAAAAGTTATCCCTGATTGGAAAAGTTGTCTACCCTACCTGGA
CGAGGGCTTGGAGGAGGAATGGAAAAGCCAAAAGTAAACCAACCCCTTTT
TCCTTCTCCGCGCAGCAGTCAGATGCAAGTGAGACAACAATACAATTGGA
ACTGTTAGCGTCCGAGATAGAGGAGCCCGACGAAAATACTCTAGTCAGCT
TTCAAAGTTTGTGTAATCACCTGCGTAGCTACTTGAAAGAC

Seq. ID No: 156

>PSIM_4ATGAGCGATATAGATGCTCAAAGTCTTGTTTACGCCCGTGCGA
ACGTGGCTCGTAATGCACTGGAAGGCAGGATTGCTGTCGTGACAGCCGAA
CCTGAAGGCTCCATCTTTGGGCCGATTGAAGCGGAGAAGGAGATACAGTT
CGACTTTACTATGTGCAATCCTCCCTTTTACGCTTCTGCGGAAGATATCG
CTCAGTCAGCAGCAACGAAGGAATTGGGTCCAAATGCCGTTTGCACCGGT
GCGGCGGTTGAAATGATAACGTCTGGGGGCGAAGGGGCGTTTGTTGCCAG
AATGATTGATAACTATGCTATAACTGAGTTCGTCCAAGGGCAAACTAGGC
GTTGGGCAATAGCATGGAGCTTTGGTCACGATAGGTTACCAGATAGCCTA
GCCAGGCTATCATCCGGCCCCCTACAATCATTGATGCCCACGAGAAATAC
ATGCCGTCGTTCTTTCACCTTTGCGAGGATGAACTTGCTTAGTAGGCTAG
AGCAAGTGCTAAATAATATAGAGGGGTTGTCCCACAGCAATATGTCTCCT
AGCGAAGATAGAGGAAGCGGGGGAAGGCCATCTAGCTTACTTGTATCCGT
TGCGAGGGATACCTGGTCCAGGGCGGCTAGGAGAAAGAAGCAGCGTGGCA
GCATGGATACATCACTAGATAACGATACATCAGGACTTATCTGTAGCGTC
AAGGTATTATTCGACGAGGAGGGCAGAGAAGGAAGCGAAATAGCATCATT
AGAATGTACTTGGATTCATGGGAGGGAACGTGCTTTATTTGAATCATTTT
GGGGACATATCTGTAGAAAAGTAGGGGAAGTTTCTGGT

Seq. ID No: 157

>PSIM_5GAGGGACTATCCCCCTTCCTGCTTATGCACCCAAGAAATCAAT
ACTGTAAAAAGAAGCCGGATTTCGCAGATCTAGCTAAAAGCCACCCTCCG
TTAAGAGAACATTTAAAGTGGAAAACTGAGGACTACGCGACTATTGATTT
CAAATCTCCATCCGCACAGAAGGAGTTGACACGTGCATTACTGAAACAAG
ACTTCCATCTGGACGTTGATATGCCTGTTGACAAATTGGTCCCAACAGTT
CCCCAGAAATTAAATTACATTCACTGGATTGAAGACCTGCTGAGCGGCGG
CCGTTCAGATTCTATCCCGCGTGGAGAAGGGATCAGGGGCATAGATATAG
GAACCGGGCCGGCGTGCATCTACCCGCTACTGGCGACTTCACTTAATAAA
TGGACCTTCGTCGCGACTGATATCGACGCTGTTTCACTAGAGTACGCTGT
TAAGAATGTGAGCCGTAATGATATGGAAGGTCGTATCAGGGTGAAAGGGG
TGGACCCAGATACGTTACTTGTCGGTGTGGTCCGTGACGAACAGTTTGAC
TTCTGCATGTGTAACCCGCCATTTTATGGGATCGATGAAGACCATCACGA
CAACCAGAGGCCCCCACCTCCCTATAGCAGCTGCAGTGCCCAGGCACACG
AAGTCCGTGTGCAGGGAGGTGAGGTTGGCTTTGTAAGTAGAATGGTTGAG
GAAAGCCTGCTGTTGCCCTCCCGTGTGCGTTGGTTTACCTCTATGGTGGG
CAAGAAAGGATCTCTTAAAAGTCTAAGGGCACTGCTGAGGAAGAGGGAGG
TGCCCACGGTAACCACGACGGAGTTCGTGCAGGGTGTAACAAAAAGGTGG
GCTGTTGCATGGAGTTTTACCGAACAAGTACCATGCATCCCTAGCCACTC
ACTTCCGTGTACGGTGCCATTACTGGGTTCTACCAGTAGCGCAGAAGGAC
GTGCATACGCTGAACAATGGTTAGAAAGGGTCCTTAACCATATGGAGGTA
ACATTCACAAAGAAGGATCAGGATGGGTACACTTGCACCGCAGAACGTGC
TACTTGGGCTAATCAGAGGAGGAAGAGGAGGCTTATGCAGCGTCCGATGA
TGTCCCCCGAGGCTGCAGCAAAGAGATCCTGCGGAGGAAGCGATAATACG
AGTGAAGGAGTCCCTAGGAATGACTCAGACACCTTAGTCAGCGCCGGACA
CCTAAGTCCGAAGGCTGACAGCCTAGAAAGGAACGCTAGTTCCGATCTTG
CGGCTCAGCTAAGTGCCTTGACACCACCGTACCACGTAACCTTCTGGTGC
GGAGTCCAACCAAGCGTACCTCCCAGCACTAACAAAGCGGAGTTAGAACT
AAAGATGGTACTGATTGACGGCGGAAGCGGTACTCAGCCGCTACAGCCCA
TTGCTCAATACATGAAGAATAACTGGAGTGCCACAGATTCAAGACCAACA
TCTGATCGTAGTAGCCAA

Seq. ID No: 158

>PSIM_6TGCATCTACCCACTGCTAGGGGCCACGATGAACGGATGGTATT
TCTTGGCGACTGAAGTGGACGACATTTGCTTCGATTATGCCACAAAGAAC
GTGGAACAAAACAACTTATCTGACTTAATTAAAGTAGTTAAAGTCCCCCA
GAAAACTTTGCTTATGGACGCATTGAAGGAGGAAACCGAAATAGTCTATG
ACTTCTGTATGTGCAACCCGCCCTTTTTCGCGAATCAGTTGGAAGCCAAG
GGGGTCAATTCCCGTAATTCCCGTAGGCCTCCTCCGTCTTCAGTAAACAC
AGGTGGAGTCACTGAGATAATGGCAGAAGGGGGTGAATTGGAATTTGTAA
AAAGAATTATTCACGACAGTCTACAGTTAAAGAAGCGTTTAAGGTGGTAT
AGTTGCATGTTAGGAAAGAAGTGTTCTCTTGCACCTCTGAAAGAGGAGTT
AAGAAAACAGGGGGTCCCTAAGGTAACCCATACTGAGTTCTGCCAAGGGC
GTACCATGAGGTGGGCCCTTGCATGGAGCTTTTATGATGACGTTATAGTG
CCCTCACCACCTAATAAAAAGAGGAAACTGGAAAGGGCAAGGAAGCCGCT
ATCTTTCACTTTACCGGAAGCCGGACTGAAGGAATTACAGAGTAAAGCAC
TTGCTCTTGGAGGCACTGCATGTAGCCCGGTGGACCGTGTAGCAGCACTT
TTGGAGAAAACTCTTACCGACCTTAGAGTCCTACATAAACGTGTTCCGTG
TAGGAAGCAAGAACAAAGCCTATTTCTAACTGCCGTTGAGAATACATGGA
TACATGGTCGTCAGAAAAGGCGTGAACAAAGTCGTCAGCTTAGAGAGCTG
CCCCGTGCGCCACCTTGCGCGGGTACATCCTCCCAAACTACAGTTGCTAC
GGCCGACTCTGTGAAAACCCCAGCATCACAGACTCAATCCGCATCTACTC
AAAATTCCAACTCCCAAGACGATAGCAGTCAGAACAAGCGTGCGAGTGCG
CAGGAATTAGCCGGTCAACAACCCACGGATAAGGCCGGCAGTAGCGCATC
TTCTGATGAAATATCAATTAAAGTGCTGCATAACTCTACGGGAGAGCAAA
AGGAGGTCACGGAAAATCTTTCAAGCGAGGCAGTAGACATGGAGTTCAGT
ACAAGCACAGAAGCAGTCCAGGAGACGGGTTCTAAAGAGGCTCCCTCTGC
TGAAAGCGAGCCTCCCAGCAAGAGACCCCTTTCTCCCGGTACGGTCGAAC
AATTTCTATTTAAATGTTTACTAAACGTAATGCTTGAGGAAAGTGACGTC
ATGATCGAAATGCATTGGGTGGAGGGTCAGAACAAGGATCTTATGAACCA
ATTATGCACTTACCTGAAAAACACTCTACTGAAGTCTGTCGCTAAATCC

Seq. ID No: 159

>PSIM_7ATGGGCTCAAAGAAGAGGCGTCGTAGACGTGAAGAGCGTCCGA
CAATTCATCCGAAGAATAAGTATAGTGAAAACCCGCCCGACTTTGCGCTA
CTAGCCTCCTTGTACCCCAGCTTCGAACCTTTTGTTTTCTATTCTCGCGA
CGGCCGTCCCCGTATAGATTGGACTGACTTCAATGCGACGCGTGAGTTAA
CAAGAGTTTTGCTGCTGCACGATCACGGCCTTAATTGGTGGATACCTGAC
GGTCAATTATGTCCCACGGTCCCGAACCGTAGTAACTACATACATTGGAT
CGAGGACCTTCTGAGTAGTAATATAATCCCGACCACTTCGAGAAATGGTG
ATAAGGTAAAAGGTTTCGATATCGGTACGGGTGCGAATTGTATTTATCCG
TTGTTGGGAGCTTCTCTACTTGGTTGGAGCTTCGTAGGGAGCGACATGAC
CGACGTGGCGTTAGAGTGGGCAGAGAAGAACGTAAAAAGCAACCCGCATA
TATCTGAACTGATCGAGATTCGTAAAGTTGACAATTCTGAATCTACCCCT
AGTATTCAAGAATCCTTAACTGGGAAATCAGTGCAAGACGAGTCTAATAT
GGACATGTCCGGCCACATGGACGAGGAGGCCGAACCAAGTTCAAGTTCTT
CTTTTAACCTTCCAGCGGGCGCTCAAAGTTCCTATCACGGACCTCCGGTT
CTGGTGGGTGTTGTAAGAGATGGCGAACAGTTCGACTTCTGTATTTGTAA
TCCGCCCTTCTTTGAAAGTATGGAAGAGGCTGGACTAAACCCCAAAACAT
CTTGCGGAGGAACTCCTGAAGAAATGGTATGTAGCGGTGGCGAAAGGGCT
TTCATAACCAGAATAATAGAAGACAGTGTTGCTCTGAAACAAACGTTCAG
GTGGTACACCAGTATGGTCGGCAGAAAAAGTAATTTAAAATTTCTTATCT
CAAAATTGAGGAAGGTCGGTGTCACAATAGTCAAAACAACGGAATTTGTC
CAGGGTCAGACGTGTCGTTGGGGTTTGGCATGGAGTTTCGTCCCGCCTGC
TCGTAAAATCATCAGTCCGCATGTTGCAGAGAAAAAGAATCTATCCTTTA
TGCTTGAATGTACGTTGATTAACAGGAGCTTATATCAGATGATCAATGTG
ACTCAAAGC

Seq. ID No: 160

>PSIM_8ATGCATCCTCGTAACCCTTATAGGCAATTGCTTGACTTCGCCA
GTTTGGCAGAGGCATACGAGCCACTAAAGCCCCACTTGAAACCTACCAGA
TCTCCCACCGCAGGCGGTTTATCATACACCATAGATTTTAAGAACTCCGA
GTCTCAGAGGCAACTGACAAAGGCAATCCTGTATAGGGATTTCGGGCTGC
GTATTGCGCTGCCAGATCACCGTCTATGTCCGCCTGTGCCAAACTCTCGT
TTAAATTACATTTTGTGGTTACAAGACATCATTAAAGCGCACGACGAGTA
TATGGATAGGCCCGCCTCTTGCATATGCGGATTAGATATAGGCACTGGAG
CAAGCGCCATCTACCTTTTGCTTGGGTGTAGAGTAGAGCCCAGTTTTAGG
TTCATAGGCACAGAACTAGACGATATATCATTCAGCTACGCCACTCAAAA
CGTCGAGTCTAACGGCCTGTCTGACAGGATACACCTGATAAAAACGACAA
GCAACGATCCCATCCTGCTACCGTTCGATCTAAATCCGGCATGGTCTTGC
GATTTTACGATGTGCAATCCCCCTTTCTACGAGAGCGAAGAGGAAATGGC
CCGTAGTGCGCAGGCGAAAGAATTAGCTCCAAATGCGGTTTGTACGGGGG
CTCAAGTGGAGATGGTTACCCCCGGTGGAGAACTAGCGTTTGTATCACAA
ATTGTCAAGGAATCTTTAAAGTACACCACCCGTTGTAGGTGGTATACTTC
AATGTTAGGTAAATTGAGCAGCTTAACAAAGTTAGTAGGACTGCTTAGAG
AGTACGCGATCTCCAATTATGCTATTACGGAGTTCGTCCAGGGGCAGACA
AGGAGATGGGCTGTGGCTTGGAGCTTCGGCGAGACACACTTGCCGGATTC
TGTGGCCAGGATTAGTAACCCTACGCTGCAACCCTTGCTGCCGGAGAGGA
ATACGAGTAGGCACGTGATCAATATCAGTCTTCCTCCGTTTAGCACAAGG
ACAGTGAAATCAAAACAGTCTATCAAGGCGCTTTCTGAGGTTCTTAGTCA
GATCAAAGATGTGACTGTGCAGAGATTATATCAAGTGGAACACCTAGAAC
CCACCGAAGAGGAAGAGGAGGACAAGAGTCTGTATAGATTGCTTGTTTAC
GCGAAGCAGAATATGTGGAGCAGGTCTGCTCGTCGTCAAAGAGGAAGGGA
GACTGGCCATAAAGCCAACGATAAAGGATGCGCTGTAGGGGGTCCTTTAA
CTTCAATTCCGGCTACTTTAGATGGATTACTTTGCGGGATAGAGATAAAG
GCACCGTTGATAAAGCAAGAGCAACAGGATGTAGAGATGGAATTTGTATT
TCAGTGGGTTCACGGGCAAGATAGAAGTATGTTTGAATCCTTCGTGAACC
ATGTAACTAGAAAGATGAAATGTAATATAGTACTGGAC

Seq. ID No: 161

>PSIM_9ATGGCTCTGAATAAATCCATGCACCCGCGTAACAGATATAAAG
ATAAGCCGCCAGATTTTGCGTTCCTAGCATCCAAGTACCCGGAATTTAAG
CAGCACGTTGACGTTGGTTTAAGTGGTAAGGTAGGACTGAATTTTAAGGA
CCCAGGCGCGGTCAGGGCTCTCACGTGTACACTTCTAAAGGAGGATTTTG
GGCTGACTATCGATATCCCCCTTGAGCGTCTTATTCCCACCGTTCCATTG
CGTCTGAACTACATACATTGGGTAGAGGACCTGATAAATTTTCATGATAG
CGACAAGACTACCGTGAGAAGAGGTATAGATATAGGCACAGGCGCAAGTT
GTATCTACCCCCTTTTAGGAGCAACATTAAATGGCTGGTATTTCCTAGCG
ACCGAGGTAGATGATATTTGCTACAATTACGCTAAGAAGAACGTGGAACA
GAACCATCTAGCCGATCTTATTAAGGTTGTCAAAGTCCCGCAAAAGACTC
TACTTATGGACGCCCTGAAGGAAGAGTCAGGAATAATATATGACTTTTGT
ATGTGCAATCCACCATTCTTTGCAAATCAAATGGAGGCTCAGGGCGTCAA
TTCCAGGAACCCCAGACGTCCTCCTCCGAGCAGCGTAAATACGGGAGGGA
TTACCGAAATAATGGCAGAAGGTGGAGAACTTGAATTTGTGAAGCGTATC
ATTCACGATTCACTACAACTGAAGAAGAGGCTTCGTTGGTATTCCTGTAT
GCTGGGGAAAAAGTGTTCTCTAGCGCCATTGAAAGAAGAGTTGAGAATAC
AGGGGGTGCCAAAGGTAGCCCACACCGAATTTTACCAAGGCCGTACTATG
CGTTGGGCGCTGGCTTGGAGTTTCTATGATGACGTAACGATCCCTAACCC
TCCGTCTAAAAAGCGCAAACTAGAGAAACCAAGGAAGCCGATGATGTTCT
CTGTTTTAGAAACTACCGTGAAAATGTTGATGGACAAGTTCGATTGTAGC
GTAGACAGTGAGCACGTCTCCGTCGTAACGGATTGTTTGAAGAAGATCCT
TACCGACTTAAAAGTCCAACATAAACCGGTTCCATGCGGTAATGGGGAGG
AGTCTCTATTTCTAACTGCGATCGAGAATAGTTGGGTTCATATAAGACGT
AAAAAGCGAGATAGGATGAGGCAACTTAGAGAATTACCCAGGGCACCTGA
TGAAAACTTTCTCCTAGTTCAAAAGGACGAAAGGCAGGCTGAGGACGAGG
AAACGACTGAAAAGACAGTGTCAAGCAGTGAGAAATCCGTCAGTACATCA
GGCATTGATGAAGCTGCCGCCCTACCCCCAAATCCGGAGGATTCCATCTC
CGAAAGTATGGGGGAAGACAGTCGTCAGCTGCCCGAGGAAGTTAAAGATA
CATCCGCACTAGGGCAGATAACCGACGTCGATGAGCACCAAAATACAATG
GAGGCGTCTCAACCTTGTTCCTCAAATAGTGCCTTCCTGTTTAAGTGCCT
AGTCAATGTTAAAAAGGAAGCTACCAATGTTTTAGTTGAAATGCACTGGG
TGGAAGGTCATAATCGTGATCTGATGAACCAACTGTGCACTTACCTAAGG
AATCAGATTTGTAAAATTGCAACGTCA

Seq. ID No: 162

>PsiHchimera_1ATGTTCTGCCGTGGCCTGTTGAGTTTAATGGCAATC
ATCATCGTGTATTTTATAGCCCAAAAGAGGAGAAGAGCCAGATTACCACC
CGGTCCTCGTGGCCTGCCACTGATCGGAAATTTGCACCAGGCACCGAAAG
AGGCGGTGTGGCTAACCTTCCATAAATGGGTGAAGGAATATGGCAACCTT
GTTTCTGTAAATTTTGGGGGCACGGAGATGGTGATACTTAATACGTTAGA
GACGATTACAGATTTGCTAGAGAAGAGGGGATCAATTTACTCTGGCAGGC
TGGAATCAACTATGGTCAACGAACTGATGGGCTGGGAGTTTGATCTTGGC
TTTATTACATATGGAGATAGGTGGCGTGAGGAAAGACGTATGTTTGCAAA
GGAGTTCAGTGAAAAGGGAATTAAACAGTTTCGTCATGCCCAAGTTAAGG
CCGCTCACCAATTAGTACAACAGTTAACTAAGACACCAGATCGTTGGGCA
CAGCACATAAGACATCAAATCGCTGCTATGAGCCTTGACATTGGCTATGG
CATTGACTTAGCTGAGGATGATCCGTGGCTGGAGGCGACGCACTTGGCCA
ACGAGGGCCTAGCAATTGCCAGCGTTCCAGGGAAATTCTGGGTTGATTCT
TTTCCAAGCCTGAAATATTTACCTGCTTGGTTCCCTGGAGCTGTTTTCAA
AAGAAAAGCGAAGGTTTGGAGGGAAGCGGCCGATCACATGGTCGATATGC
CCTACGAGACTATGAGAAAGTTGGCTCCTCAGGGGCTTACGCGTCCGAGT
TACGCTAGTGCAAGATTACAAGCGATGGATCTTAATGGCGATTTAGAGCA
CCAGGAGCACGTAATCAAGAACACGGCGGCGGAAGTAAACGTTGGCGGAG
GTGATACTACTGTGTCCGCAATGTCCGCCTTCATCTTGGCAATGGTTAAA
TACCCTGAAGTCCAAAGAAAAGTTCAAGCAGAATTGGATGCTTTAACTAA
CAATGGGCAAATCCCGGATTATGATGAAGAAGACGACAGTTTACCCTATC
TAACGGCGTGTATTAAAGAACTGTTTAGGTGGAATCAAATAGCCCCGCTG
GCAATTCCACATAAACTTATGAAAGACGACGTGTATAGAGGCTACCTTAT
CCCCAAAAATACGCTAGTCTTCGCAAACACATGGGCTGTTCTAAACGATC
CCGAAGTCTATCCAGATCCTAGTGTTTTTAGACCAGAACGTTACTTAGGA
CCAGACGGAAAACCAGACAACACAGTACGTGATCCTCGTAAGGCAGCATT
TGGCTATGGCAGACGTAATTGCCCGGGCATACATCTTGCGCAGAGTACCG
TGTGGATAGCTGGGGCCACCCTTTTGTCAGCCTTTAATATTGAAAGGCCC
GTCGATCAGAACGGTAAACCCATAGACATACCTGCTGACTTCACGACCGG
GTTTTTCCGTCATCCAGTTCCCTTCCAATGCCGTTTCGTACCTAGAACGG
AACAGGTATCTCAAAGCGTAAGCGGACCT

Seq. ID No: 163

>PsiKchimera_1ATGAAAACCAAATTTTGTACCGGCGGCGAAGCCGAG
CCATCACCATTAGGGTTACTATTGAGCTGTGGTAGTGGCCTTGTCCCTCG
TGGTTCTCCACAACCGCCAGCTGACGAGCAACCCGAGCCGCGTACCAGAA
GGAGAGCATATCTATGGTGCAAGGAGTTCTTGCCGGGGGCATGGCGTGGA
CTTAGGGAAGACGAGTTTCATATCAGTGTTATCCGTGGCGGTTTATCCAA
TATGTTATTCCAATGTTCCTTGCCCGACACGACTGCTACCTTAGGCGACG
AACCAAGAAAGGTGCTTCTGAGATTATACGGCGCTATCCTGCAGATGAGG
AGTTGTAATAAGGAAGGATCTGAACAGGCTCAGAAAGAGAATGAGTTTCA
GGGAGCGGAAGCGATGGTCTTAGAGAGCGTAATGTTCGCCATCCTGGCAG
AGCGTTCTCTAGGTCCAAAGTTGTATGGGATCTTTCCACAAGGCCGTCTG
GAGCAGTTCATAAAGATGAAAACGTTGTTAGACTATGTTACTGCAAAGCC
GCCTTTAGCGACCGACATTGCTAGATTGGTGGGCACGGAAATAGGTGGTT
TCGTGGCACGTCTTCACAACATCGGACGTGAGAGACGTGATGACCCTGAG
TTTAAATTTTTCTCAGGTAATATCGTAGGTAGAACAACGAGTGACCAGCT
GTATCAAACTATCATTCCTAATGCGGCAAAGTATGGGGTCGATGATCCAT
TGTTACCTACCGTCGTAAAGGATCTGGTTGACGACGTAATGCATTCAGAG
GAGACTTTGGTCATGGCGGACTTGTGGAGCGGTAACATTTTACTGCAGTT
AGAGGAAGGTAACCCCTCCAAGCTGCAGAAAATCTACATTCTGGACTGGG
AACTATGTAAATACGGTCCTGCGAGTTTGGACCTTGGTTACTTCCTTGGG
GATTGCTACTTGATATCTAGGTTCCAAGATGAGCAAGTCGGGACTACAAT
GCGTCAGGCGTACTTACAGTCTTATGCTAGGACCAGCAAGCACTCCATCA
ACTACGCAAAGGTTACGGCAGGCATAGCAGCCCACATCGTTATGTGGACT
GATTTTATGCAATGGGGGAGTGAGGAGGAAAGGATCAACTTCGTCAAAAA
GGGGGTGGCGGCATTCCACGACGCTAGAGGGAATAACGACAACGGAGAAA
TCACTTCGACCCTGCTTAAAGAGTCATCAACAGCG

Seq. ID No: 164

>PsiKchimera_2ATGGCGTTCGACCTAAAGACCGAAGATGGTCTAATC
ACATATCTGACAAAGCACCTGTCTTTAGATGTAGATACCAGCGGAGTTAA
ACGTCTATCCGGAGGGTTTGTTAACGTCACCTGGAGAATAAAACTTAATG
CACCGTACCAAGGACACACGAGTATCATCCTAAAACACGCACAACCCCAT
ATGTCAACGGATGAAGACTTTAAGATCGGAGTTGAGCGTAGCGTCTACGA
ATATCAAGCGATTAAGTTGATGATGGCAAACCAGGAGGTGTTGGGTGGAG
GCGACAGCCGTGTTAGCGTCCCCGAAGGCTTTCACTACGACGTTGAAAAT
AACGCATTAATCATGCAGGATGTTGGAACTATGAAAACTCTGTTAGACTA
TGCAACTGCAAAACCGCCACTTTCAACGGAAATCGCGAGTCTAGTGGGGA
CCGAAATCGGGGCCTTTATCGCGCGTCTTCACAACCTAGGTCGTAAACGT
CGTGACCAACCGGCTTTCAAATTCTTCTCCGGAAATATTGTAGGCCGTAC
AACAGCCGATCAGCTTTATCAAACCATTATTCCAAATGCGGCCAAGTACG
GTATTAACGACCCACTACTACCCACTGTTGTAAAAGATCTTGTCGAAGAG
GTTATGAACAGTGAGGAAACCCTGATTATGGCAGATTTATGGAGCGGGAA
CATTCTACTGCAATTAGAGGAAGGGAACCCTTCAGAGTTGAAAAAGATCT
GGCTTGTTGACTGGGAATTGTGTAAGTACGGACCAGCCAGCCTAGACATG
GGGTACTTCCTAGGAGACTGCTACCTTATTGCCAGATTTCAGGACGAGTT
GGTAGGTACGACTATGAGAAAAGCCTATCTAAAATCTTATGCGAGAACCG
CTTCAGACACTATAAATTACTCAAAGGTCACGGCAAGCATCGGGGCGCAT
CTAGTTATGTGGACCGATTTTATGAAGTGGGGAAATGACGAAGAAAGGGA
GGAA

Seq. ID No: 165

>PsiMchimera_1ATGGATAGCGCAGGAAACATTTACCGTCACAAAGTA
GACTTCACCGCGCTTGCGCTTCAGGACCCTGCCTTCAAGGAGACGCTATC
AGCAAAGGGACGTTTAGACTTCAGCAATCCAGATGCGGTTAGACAACTTA
CTGTGAGTCTGTTGAGGAGGGATTTCGGGTTGGAGGTTGAGCTTCCAGAC
GACAGGCTTTGCCCTCCCGTGCCAAACAGGCTTAACTACATTTTGTGGTT
GCAAGACTTGATAGATTGTACAGGGGATGATTACCACGAGGGCTTTAATG
CTGACCGTGATGTGGTGGGACTGGACATAGGGACGGGGTCTAGCGCCATT
TATCCTATGTTGGCTTGCGCAAGATTCAAGGCTTGGAGCATGGTCGGAAC
AGAGGTAGAAAGAAAATGTATAGATACGGCCAGACTAAATGTCGTCGCCA
ATAACTTGCAAGACAGACTGTCCATTTTGGAGACTTCGATTGATGGGCCC
ATCCTAGTACCCATTTTTGAGGCCACTGAAGAGTATGAGTACGAGTTCAC
CATGTGTAACCCGCCCTTTTACGACGGGGCTGCCGATATGCAGACATCTG
ACGCGGCGAAAGGGTTTGGCTTTGGGGTGGGGGCTCCTCATTCCGGTACA
GTGATTGAGATGAGTACGGAAGGGGGCGAGAGCGCATTTGTAGCACAAAT
GGTTCGTGAGAGTCTGAAGCTGAGGACTAGGTGCAGGTGGTACACCTCAA
ATCTAGGCAAGCTGAAAAGCCTTAAAGAGATCGTAGGGCTTTTAAAAGAG
TTGGAAATATCAAACTATGCTATCAACGAATATGTCCAAGGAAGTACAAG
AAGATATGCGGTGGCTTGGTCTTTTACAGACATTCAACTACCCGAAGAAC
TTTCAAGGCCATCTAATCCTGAACTTTCAAGCCTTTTT

Seq. ID No: 166

>PsiMchimera_2ATGAGCGCCACCACCAACATATACAAGGAAGACATT
GACTTTATAACTTTAGGGAGGGAAGATAGCGACTTTGGAAAGTTACTAAA
CTCAAACGGGCAATTAGATTTCTCTGACCCCAAATCAGTACAACAGTTGA
CTAAGTCTCTGTTAAAGAGGGATTTCGGCCTTAAATTGATTCTTCCCGAT
GACAGGTTATGCCCACCTGTTCCCAACAGGTTAAATTACGTACTATGGAT
AGAAGATATTTTCAACTACACAAACAAAACCCTAGGCCTGAGTGACGACA
GACCAATCAAAGGAGTTGATATCGGAACAGGTGCTTCTGCTATCTATCCT
ATGCTGGCCTGTGCCCGTTTCAAGGCTTGGAGTATGGTTGGTACGGAGGT
CGAGAGGAAGTGTATTGATACGGCTAGATTAAACGTTGTCGCAAACAATT
TACAAGACAGATTATCCATTCTGGAGACTTCCATAGATGGACCTATTCTT
GTGCCTATTTTTGAGGCCACCGAGGAATATGAGTATGAGTTCACAATGTG
TAACCCGCCGTTTTATGACGGCGCGGCGGACATGCAAACGAGCGACGCAG
CAAAGGGGTTCGGTTTTGGAGTTGGCGCTCCACATAGTGGGACCGTTATC
GAAATGTCAACAGAAGGCGGCGAGTCCGCTTTCGTGGCTCAAATGGTGAG
GGAAAGCCTAAAGTTGAGGACCCGTTGTCGTTGGTATACTTCTAATCTTG
GCAAGTTAAAGTCTTTGAAGGAAATTGTAGGTCTATTAAAGGAGCTAGAA
ATTTCCAACTACGCGATCAATGAGTACGTGCAGGGCTCCACAAGAAGGTA
TGCCGTCGCTTGGTCCTTTACGGATATCCAATTACCAGAAGAGTTGTCAC
GTCCGTCCAACCCAGAGCTGTCAAGCCTATTT

Seq. ID No: 167

>PsiMchimera_3ATGGCTCAAAACTCCACCATCTATGAAGATGAAGTA
GATTTTGCCACACTAGCATTGCAAGATTCCGAGTTCGCGAAGATTCTGAA
ATCTAACGGACAGTTGGATTTTAGTAACCCCGAATCCGTGCAGCAGTTGA
CAAAGTCATTGCTAAAAAGGGATTTCAAGCTGAAACTTTCTCTGCCTCCG
GATAGACTTTGCCCGCCCGTTCCTAACAGGCTTAACTACATTATATGGAT
TCAGAATCTTCTTGACACTACGTCTGATTCATATAACGACAAGTACGATC
CCGAGCGTGAGGTTCTTGGATTAGACATCGGCACGGGCGCCAGTGCAATT
TACCCCATGCTTGCTTGCGCTCGTTTCAAAGCGTGGTCAATGGTCGGGAC
CGAAGTTGAGAGAAAGTGCATCGATACTGCCAGATTGAATGTGGTAGCGA
ACAATTTACAAGATAGGCTAAGCATTTTGGAGACATCCATTGATGGTCCG
ATTTTAGTACCCATTTTTGAGGCGACAGAGGAGTACGAGTACGAATTTAC
GATGTGCAATCCGCCATTCTACGACGGGGCAGCAGACATGCAGACCTCTG
ATGCTGCCAAAGGATTTGGCTTCGGTGTTGGCGCCCCACACAGTGGTACG
GTGATAGAAATGAGCACTGAAGGAGGGGAGAGCGCATTTGTCGCTCAAAT
GGTCAGGGAGTCACTAAAACTGAGGACCAGATGTAGATGGTATACGTCAA
ACTTGGGAAAATTGAAATCTTTGAAAGAAATTGTGGGTCTGCTTAAAGAA
TTAGAAATTTCTAACTATGCTATAAACGAATATGTTCAGGGTAGCACGAG
AAGGTATGCCGTGGCCTGGTCATTCACGGATATTCAATTACCCGAAGAGC
TTTCACGTCCCTCTAATCCCGAACTGTCCAGCTTGTTC

Seq. ID No: 168

>SAM2ATGTCTAAATCCAAGACTTTCTTATTTACTAGCGAGAGCGTAGGA
GAAGGGCACCCAGATAAAATCTGTGACCAGGTATCTGATGCAATTCTGGA
TGCCTGCCTAGAACAAGACCCCTTCTCTAAGGTAGCCTGCGAAACCGCCG
CAAAGACTGGAATGATTATGGTCTTCGGCGAAATTACTACAAAGGCAAGA
CTAGATTATCAACAAATCGTTAGAGATACTATCAAAAAGATCGGGTATGA
TGACTCCGCGAAAGGATTTGATTATAAGACGTGTAACGTACTTGTAGCAA
TTGAGCAGCAAAGTCCAGACATTGCTCAGGGGCTGCATTACGAGAAGTCT
CTTGAGGATTTAGGCGCAGGCGATCAGGGCATTATGTTCGGGTATGCTAC
CGACGAGACGCCGGAGGGCCTTCCGTTAACGATATTGCTGGCGCATAAGC
TAAACATGGCTATGGCCGACGCACGTCGTGATGGGTCACTACCGTGGCTG
AGGCCAGACACCAAAACTCAGGTGACCGTCGAATATGAAGATGACAACGG
TAGATGGGTTCCGAAGCGTATTGACACGGTGGTTATCTCCGCCCAACACG
CAGATGAGATCTCTACTGCGGATCTAAGGACTCAATTGCAGAAGGACATT
GTCGAAAAGGTAATTCCGAAAGATATGCTAGATGAGAACACTAAGTATTT
TATCCAGCCAAGCGGGAGATTTGTAATAGGTGGGCCGCAGGGTGATGCCG
GCTTAACAGGTAGGAAAATAATAGTCGATGCCTACGGCGGAGCCTCAAGC
GTGGGTGGTGGAGCATTTTCTGGGAAGGACTACTCAAAAGTTGATAGATC
TGCAGCGTATGCAGCGAGGTGGGTAGCCAAGTCCCTAGTGGCGGCCGGAT
TGTGCAAGAGAGTTCAAGTGCAATTTTCCTACGCCATTGGTATTGCAGAA
CCCTTGAGCTTGCATGTGGATACCTACGGCACCGCGACAAAGAGTGATGA
CGAAATAATAGAAATTATTAAGAAGAATTTTGATCTGAGACCTGGCGTTC
TGGTCAAGGAGTTAGACCTGGCCAGGCCAATATATCTACCGACCGCGTCA
TATGGGCACTTCACAAACCAGGAGTATTCATGGGAAAAGCCCAAGAAACT
TGAGTTC

Seq. ID No: 169

>SAM3ATGGACATCCTGAAAAGAGGTAACGAGAGTGACAAGTTTACTAAG
ATAGAGACCGAGTCAACCACGATTCCGAACGACTCCGATAGAAGTGGATC
TCTTATTAGACGTATGAAAGATAGTTTCAAGCAGAGCAACCTGCATGTAA
TCCCGGAGGACCTTGAAAACAGCGAACAAACTGAGCAGGAGAAGATCCAA
TGGAAGTTGGCTAGTCAACCTTACCAGAAAGTGTTATCACAACGTCACCT
GACCATGATTGCAATAGGCGGCACTTTAGGTACTGGACTGTTTATCGGCC
TAGGTTATTCTCTAGCTTCCGGTCCTGCCGCGCTTCTTATTGGCTTTTTG
CTGGTTGGCACCAGCATGTTTTGTGTTGTCCAATCCGCTGCGGAGTTGTC
CTGCCAATTCCCCGTCAGTGGCAGCTATGCGACCCATGTCTCTAGGTTCA
TTGATGAATCCGTGGGCTTTACGGTAGCAACCAATTACGCTCTTGCGTGG
TTGATATCCTTTCCCTCCGAACTGATCGGGTGTGCCTTAACCATTAGCTA
TTGGAACCAGACCGTAAATCCGGCTGTGTGGGTGGCAATTTTCTACGTAT
TCATAATGGTGCTGAACCTTTTCGGAGTTAGGGGTTTTGCAGAGACAGAG
TTTGCTCTGTCTATCATAAAAGTAATTGCGATATTCATATTCATCATTAT
TGGGATTGTATTGATAGCAGGTGGTGGTCCTAATAGCACTGGCTATATAG
GCGCGAAGTATTGGCACGACCCAGGTGCGTTCGCTAAACCGGTATTCAAG
AATCTATGCAACACATTCGTGAGTGCTGCGTTTAGCTTCGGTGGTTCAGA
ACTGGTATTATTGACCTCAACAGAAAGCAAGAATATCAGCGCAATAAGCC
GTGCGGCTAAAGGCACTTTTTGGAGGATAGCGATATTTTATATCACCACG
GTTGTGATTATTGGGTGCTTAGTCCCTTACAACGATCCCAGACTACTTTC
TGGGTCTAATAGCGAAGACGTCTCCGCCTCTCCTTTCGTCATAGCTCTGT
CTAACACAGGCTCAATGGGAGCCAAGGTCAGTAATTTTATGAATGTCGTG
ATTTTAGTAGCAGTGGTGTCAGTATGCAATTCATGCGTCTATGCGAGCAG
TAGGTTAATTCAAGCCCTGGGCGCCAGCGGGCAATTGCCTTCTGTGTGTT
CTTATATGGATAGGAAAGGGAGACCGCTTGTTGGTATCGGCATAAGTGGA
GCCTTTGGTTTACTTGGATTTTTGGTGGCGAGCAAAAAGGAAGATGAGGT
GTTTACGTGGCTATTTGCCTTATGCTCTATCAGCTCCTTCTTTACCTGGT
TCTGCATCTGCATGTCCCAGATCAGGTTCAGAATGGCGTTAAAAGCTCAA
GGGAGGAGTAACGATGAAATAGCCTATAAAAGTATACTAGGTGTATATGG
GGGTATCTTGGGGTGCGTATTGAACGCTCTTCTAATTGCGGGGGAAATCT
ATGTGTCTGCGGCACCCGTCGGATCTCCCTCTTCTGCTGAGGCATTTTTC
GAATATTGCCTGAGTATTCCCATAATGATCGTAGTTTATTTTGCCCATCG
TTTTTATAGGAGGGATTGGAAACACTTCTATATCAAGAGGTCAGAAATCG
ACCTTGACACGGGGTGTAGCGTCGAAAATCTTGAGTTATTCAAAGCCCAG
AAAGAAGCTGAGGAACAGCTAATTGCATCTAAGCCGTTCTACTACAAAAT
TTACAGGTTCTGGTGT

Seq. ID No: 170

>SS02ATGTCTAACGCGAATCCTTACGAGAACAATAATCCATACGCTGAA
AATTACGAAATGCAGGAAGACCTGAACAATGCCCCAACGGGGCACAGCGA
CGGCTCAGATGACTTCGTCGCATTTATGAATAAGATTAACTCAATCAATG
CTAATCTATCAAGATACGAGAATATTATTAACCAAATTGACGCGCAACAT
AAAGACTTACTTACCCAGGTCAGCGAAGAGCAGGAAATGGAGCTGCGTCG
TAGTCTCGACGACTACATAAGCCAAGCAACAGATTTGCAGTACCAACTAA
AGGCAGACATAAAGGACGCGCAAAGAGACGGACTACACGACTCAAATAAA
CAGGCTCAGGCGGAGAACTGTAGGCAAAAATTTCTGAAGTTAATTCAAGA
CTACAGAATTATTGATTCTAACTATAAGGAGGAATCAAAAGAGCAAGCAA
AGAGACAGTACACGATTATCCAACCAGAAGCAACAGATGAAGAGGTTGAA
GCAGCCATCAACGATGTAAACGGGCAGCAAATATTCTCTCAAGCATTGCT
AAATGCTAACAGGCGTGGCGAAGCCAAGACCGCCTTAGCTGAAGTCCAAG
CTCGTCATCAAGAGTTATTAAAGTTGGAGAAAACTATGGCTGAATTGACA
CAGTTATTTAACGACATGGAAGAGTTGGTCATCGAACAGCAGGAGAATGT
GGATGTCATTGACAAGAACGTCGAGGATGCCCAGCAAGATGTGGAGCAGG
GGGTCGGGCATACAAATAAAGCTGTAAAATCTGCGAGGAAGGCAAGAAAG
AACAAAATCCGTTGTCTAATAATTTGCTTTATCATATTTGCTATAGTCGT
AGTAGTTGTTGTTGTCCCCTCTGTTGTCGAGACGAGGAAA

Seq. ID No: 171

>T4H-CPR_1TCTAGCTCTAGTGACGTGTTCGTACTGGGGTTAGGAGTCG
TACTAGCAGCTCTTTACATATTTCGTGATCAGCTTTTCGCTGCCTCCAAG
CCAAAGGTGGCACCGGTGAGCACAACGAAACCGGCTAATGGGAGCGCAAA
TCCTAGAGATTTTATCGCCAAGATGAAACAGGGAAAGAAACGTATTGTTA
TTTTCTACGGCTCTCAGACAGGCACCGCGGAAGAATATGCAATACGTTTA
GCAAAGGAGGCAAAACAAAAATTTGGACTAGCATCCCTAGTATGTGACCC
AGAAGAATATGACTTCGAGAAGCTAGATCAGTTGCCTGAGGACTCTATAG
CATTCTTCGTAGTAGCCACATATGGAGAGGGTGAACCGACAGATAATGCG
GTTCAACTATTACAAAACTTACAGGACGAATCATTCGAGTTCTCCTCAGG
GGAGCGTAAGTTGTCAGGACTTAAATATGTTGTCTTCGGCTTGGGCAATA
AGACCTACGAGCATTATAATCTAATAGGGCGTACTGTAGACGCGCAGCTT
GCTAAAATGGGAGCGATCAGAATCGGAGAAAGGGGTGAAGGAGACGACGA
CAAATCTATGGAGGAGGACTACTTGGAATGGAAGGATGGAATGTGGGAGG
CGTTCGCGACAGCGATGGGTGTCGAGGAGGGGCAGGGAGGTGATAGCGCC
GACTTCGTGGTAAGCGAATTAGAGTCACATCCACCTGAAAAGGTCTATCA
AGGCGAGTTCTCTGCGAGGGCTCTAACTAAGACTAAAGGCATTCATGACG
CGAAGAACCCTTTCGCGGCTCCCATCGCAGTAGCGCGTGAACTGTTCCAG
AGCGTAGTAGACAGGAACTGCGTGCATGTGGAATTTAACATAGAAGGGAG
TGGAATAACCTACCAGCACGGCGATCATGTCGGCTTGTGGCCGTTGAATC
CTGACGTAGAGGTGGAGCGTTTATTATGCGTTCTGGGTTTAGCGGAGAAA
AGAGACGCAGTGATCTCCATTGAGTCCCTGGACCCAGCCCTTGCGAAAGT
GCCATTCCCTGTTCCAACGACCTATGGAGCGGTCCTTAGACATTACATTG
ACATATCTGCCGTCGCCGGAAGGCAGATTCTTGGAACTCTATCAAAATTC
GCACCAACTCCAGAGGCTGAGGCGTTTTTAAGGAACTTAAATACGAACAA
GGAGGAATACCACAATGTCGTCGCGAACGGGTGTTTAAAACTGGGCGAAA
TCCTGCAGATAGCGACTGGTAATGACATCACAGTACCGCCTACGACCGCC
AATACCACTAAGTGGCCGATACCCTTTGATATAATCGTATCCGCTATTCC
CAGACTACAGCCTCGTTACTACAGTATCAGTTCTAGTCCAAAAATCCATC
CGAACACAATACATGCGACGGTCGTGGTACTGAAGTATGAAAATGTTCCC
ACTGAACCTATCCCAAGAAAGTGGGTGTATGGTGTAGGGTCAAATTTCTT
ACTTAACCTGAAGTATGCAGTAAATAAGGAACCGGTTCCATACATAACGC
AAAACGGGGAACAGAGGGTCGGAGTTCCCGAGTACCTGATCGCAGGCCCT
AGAGGCTCCTATAAGACTGAGAGCTTTTACAAAGCTCCAATCCACGTGCG
TAGGTCAACATTTAGGTTGCCAACAAACCCCAAGAGTCCCGTAATCATGA
TCGGGCCAGGGACCGGAGTAGCACCCTTTCGTGGATTTGTGCAGGAGCGT
GTTGCATTGGCAAGAAGGTCCATAGAGAAAAACGGCCCAGACTCTTTGGC
GGATTGGGGCAGAATTTCACTTTTCTATGGATGTCGTCGTAGTGACGAGG
ACTTCTTATACAAGGACGAATGGCCTCAGTATGAAGCCGAACTAAAGGGA
AAGTTTAAATTACACTGTGCTTTTTCCCGTCAAAATTACAAGCCTGACGG
GTCAAAAATCTATGTGCAGGATCTGATATGGGAAGACAGAGAACACATTG
CAGATGCAATACTAAATGGCAAAGGATATGTGTATATTTGCGGGGAGGCA
AAGTCAATGTCTAAGCAGGTCGAGGAAGTCCTTGCGAAGATTCTTGGAGA
AGCTAAGGGTGGTTCAGGTCCCGTAGAAGGCGTTGCTGAGGTAAAGCTAT
TGAAGGAGAGGTCTAGGCTTATGCTGGATGTATGGTCA

Seq. ID No: 172

>T4H-CPR_2TCATCTAGCTCTGACGTTCTGATTTTGGGCCTTGGCGTTG
CGCTGGCTGCTCTTTACCTGTTCAGGGATCAGCTGTTCGCTGCTTCTAAG
CCAAAGGCTATACCGCTTACGAACAAACTGGCCGGTCTTGACAACGAAGG
GAACCCGCGTGATTTCATAGCAAAGATGAAGGCAGGAAAGAAGCGTTTGG
TCATATTTTACGGCTCTCAAACAGGAACCGCTGAAGAGTACGCGATCAGG
CTGGCCAAAGAAGCCAAATCTAAGTTCGGTTTGACCTCCCTTGTGTGCGA
TCCTGAAGAGTACGATTTCGAGAATCTGGATCAGTTGCCGGAAGAGTGCG
CCGTATTTTTCGTGATGGCTACGTACGGGGAAGGTGAGCCGACGGATAAT
GCGGTTCAGCTTATGCAGAATCTAGCCGACGAATCCTTCGAGTTCTCAGG
CGGAGAAAGGAAATTGGAGGGTTTGAAATATGTAATATTCGCTTTAGGAA
ACAAAACTTATGAGCACTACAATTTGATTGGTCGTAAAGTTGACACTCTT
CTGACTGACATGGGTGGGGTCCGTTGCGGGGAACTAGGGGAAGGCGACGA
TGACAAGAGTATGGAAGAGGATTATTTGGAGTGGAAAGACGCGATGTGGG
AGGACTTCGCGAGGAAGATGGGCGTCGAAGAGGGCCAGGGTGGTGATTCT
GCTGATTTTGCAGTCAGCGAATTAGACACTCACGTGCCCGAGAAGGTGTA
TTTAGGAGAGTTGAGTGCTAGGGCATTGACGAAGACGAAAGGGATACATG
ATGCCAAGAATCCGTATCCGGCGCCCATCGTAGCAAGTCGTGAATTGTTC
CAGCAAGGTGGCGACAGGAATTGTGTTCATGTTGAACTATCCATTGAAGG
CTCAGGAATTACCTATCAGCACGGCGATCACGTCGGAGTATGGCCCACGA
ATCCTGAGGTGGAAGTCAATAGGCTGCTTTGCGCCCTGGGTTTATGGGAG
AAGAAGGACCAGGTTATAGGGATTGAATCCCTTGACCCAGCCTTGGCAAA
AGTCCCATTTCCTGTCCCAACTACGTACGCCACTGTGCTGAGGAATTACA
TTGACATCAGCGCAGTTACGGGGCGTCAAATCCTAGGCCACTTGAGTAAA
TACGCGCCAGCTCCCGACGTCGAGGAATTTCTGAAGGGTTTGTCTACAAA
CAAGGAACAATACGGTGCTACAGTAGCCAATGGTTGCTTGAAGTTAGGCG
AGGTCCTACAACTTGCCGCGGGCAATGATCTGAAGGCTATACCAACGACT
GAGAATACTACAGCGTGGAGTATCCCCTTCGACGTTATCGTGTCCGCCAT
TCCCCGTCTTCAACCACGTTATTACTCAATTTCATCATCCCCAAAGCTAA
ACCCCACCTCAATTCACGTAACCGCTGTTGTGTTGAAGTATCAATCAGTC
GCGAGCGAAAAACTGCCTGCTAAGTGGGTCTACGGCGTAGGTAGCAACTT
CTTACTAAATTTAAAATACGCTGCTAATGGTGAACCAGCGCCATTCGTTA
CTACCAATGGCTCAGCCGATCCTGCATCAGTTTATTATCCCACTTACGCA
ATCGAGGGCCCACGTGGCGCGTATAAACAGGAAACTATATACAAATCCCC
TATACACGTGAGAAGAAGTACATTCCGTCTGCCCACGAACCCTAAATCAC
CGGTGATTATGATCGGTCCGGGTACCGGTGTGGCGCCTTTCAGGGGGTTC
GTACAAGAGAGAGTTGCCTTAGCAAGGAGAACGATTGAGAAAAATGGAGC
CGACGCGTTGGCTGACTGGGGCCGTATATCTCTTTTCTATGGGTGTCGTA
AATCAACAGAGGATTTCTTATATAAAGAAGAGTGGCCGCAGTATACAGAG
GAGTTAAAAGGGAAATTTAACATGCATAGTGCCTTTAGTAGAGAAGCACC
TTACAAGGCTGACGGAAGCAAGATTTATGTCCAGGACCTGATCTGGGAAG
ATCGTGCCAATGTGAGCGATGCGATACTGAATGGCAAGGGATATATCTAC
ATATGCGGTGATGCGAAATCAATGGCGAAACAGGTGGAAGACACGCTTGC
TAAGATACTAGGAGAAGCGAAGGGTGGTACCGCGGAAGTCGAGGGAGCGG
CGGAAATGAAGCTGTTAAAAGAAAGGTCTAGGCTAATGTTAGATGTTTGG
TCA

Seq. ID No: 173

>T4H-CPR_3TCTAGTTCTTCCTCAGGCGCCGGGGCAGATAGCGACGAAA
ACCCTCGTGATTTCATTGCCAAAATGAAAGCAGGAAAGAAGAGGTTGGTC
ATCTTCTACGGCAGCCAAACAGGCACTGCCGAAGAGTATGCGATCCGTTT
GGCGAAAGAGGCAAAATCCAAGTTTGGACTAACGTCTCTAGTGTGCGACC
CAGAAGAATATGATTTTGAAAACCTTGACCAGTTGCCTGAGGATTGTGCC
GTGTTCTTTGTAATGGCCACTTATGGAGAAGGCGAGCCTACCGACAATGC
GGTGCAGCTTATGCAGAACTTGCAAGACGAGAGCTTTGAGTTCTCTAATG
GCGAGAGAAAGTTGGAAGGGTTGAAATACGTAGTATTCGCATTGGGTAAC
AAAACCTACGAGCATTATAACTTAATAGGAAGAAAAGTGGACACAATATT
GGGGGAAATGGGGGCGGTGAGGTGCGGAGAGAGAGGCGAAGGAGATGACG
ACAAATCAATGGAGGAAGATTATTTAGAGTGGAAGGACGCTATGTGGGAA
GACTTTGCTCGTAAGATGGGGGTTGAAGAGGGACAGGGCGGAGATAGCGC
CGATTTTGCTGTTTCAGAGCTTGAGTCCCATGCTCCAGAGAAAGTATACC
TAGGTGAATTGTCTGCGCGTGCCTTGACCAAGACTAAAGGCATTCACGAC
GCTAAAAATCCATACCCCGCCCCTATCGTCGAGAGTAGAGAACTATTCCA
AGTTGGTGGGGACAGGAATTGTGTACATGTAGAGCTAGGCATTGAGGGAT
CAGGGATTACCTACCAGCACGGAGACCATGTCGGTGTGTGGCCTACAAAT
CCTGAAGTTGAGGTGACACGTCTATTATGCGCGCTGGGGTTGTGGGAGAA
AAAGGATCAAGTTATTGGAATAGAGAGCTTAGACCCTGCGTTGGCAAAGG
TGCCATTCCCCGTACCTACAACATACATAACTGTGCTACGTAATTATATA
GACATTTCTGCGGTCACTGGGCGTCAAATCTTAGGGCATCTAAGTAAGTT
TGCGCCGAGTCCAGATGCAGAGGCGTTTCTGAAATCCTTATCTACTAACA
AGGAACAGTATGGCGCCATTGTAGCGAACGGATGTCTAAAACTTGGAGAA
GTTCTGCAACTAGCCGCTGGGAATGATTTGAAAGCCGTTCCGAACGCTGA
AAACACAACGAAATGGACAATCCCGTTTGACGTTATAGTAAGTGCTATTC
CAAGATTACAGCCTAGATACTATAGTATAAGTTCTTCTCCGAAACTTAAT
CCTACGACAATACACGTCACCGCCGTAGTCCTAAAGTACGAATCAGTGGC
CTCTGAGAAAGTTCCGGCAAAGTGGGTATATGGCGTAGGGTCAAACTTTC
TATTGAACCTGAAGTATGCCGCGAATGGCGACGCAGCACCATTTGTCACA
GCCAACGGTTCAGCCGATCCGGCCTCCGTTTATGCGCCGACCTATGCCAT
AGAAGGACCCAGAGGGGCTTATAAGCAGGAAACCATTTACAAGTCTCCTA
TTCATGTAAGGAGAAGTACCTTTAGATTGCCGACAAACCCTAAGTCCCCG
GTGATCATGATAGGTCCCGGCACTGGCGTGGCACCATTCCGTGGCTTTGT
ACAGGAAAGGGTTGCACTAGCCAGGCGTACGATTGAGAAGAATGGTCCAG
ATGCGCTTGCAGACTGGGGCAGAATAACATTATTTTACGGCTGTAGAAAG
TCTACCGAAGATTTCCTATATAAAGATGAATGGCCTCAATACACCGAAGA
ATTGAAAGGGAAATTTACCATGCATAGTGCGTTCTCTAGGGAACCCCCGT
ACAAGGCGGACGGGAGTAAAATTTATGTACAGGACCTTATCTGGGAAGAT
CGTGAGAAAGTCGCGGATGCTATACTTAATGGCAAGGGGTATGTTTATAT
ATGCGGAGACGCAAAATCAATGGCAAAGCAGGTGGAAGACACCCTAGCCA
AGATACTTGGAGAGAGTAAGGGCGGTTCAGCAGAGGTAGAAGGTGCAGCA
GAAATGAAACTACTGAAGGAGAGGAGTAGACTAATGTTAGACGTCTGGTC
T

Seq. ID No: 174

>T4H-CPR_4TCATCAAGTAGCAGCTCCAAGTTGAGCGATGGCGACGAGA
ATCCTAGAGACTTCATCGCAAAAATGAAGAATGGGAAGAAACGTCTTGTC
ATATTCTACGGGTCCCAGACGGGGACTGCAGAAGAATATGCTATAAGACT
AGCCAAAGAAGCCAAAAGCAAATTTGGTCTTACCTCCTTAGTATGTGATC
CCGAGGAGTATGACTTCGAGAATCTGGACCAGTTACCGGACGACTGCGCA
GCGTTTTTCGTCGTAGCAACGTATGGGGAAGGTGAACCTACCGACAATGC
TGTACAACTAATGCAGAACCTACAAGACGAATCATTCGAATTTTCTGGCG
GTGAAAGAAAGTTGGAAGGATTGAAGTACGTCGTATTTGCCCTGGGCAAT
AAGACTTATGAACACTATAATGTTATTGGGCGTATAGTAGACACGGAGCT
TGCAAAAATGGGCGCTATACGTTGTGGGGAGAGGGGTGAGGGTGATGACG
ACAAGTCTATGGAAGAGGATTACTTAGAGTGGAAAGATGGAATGTGGGAG
GAATTTGCCCGTATCATGGGAGTTGAAGAGGGTCAGGGAGGTGACACCCC
AGACTTCAAAGTGACAGAGCTGCAATCACATCCTAGTGAGAAGGTATATT
TGGGCGAGCTTTCCGCGAGAGCACTGACAAAGACTAAGGGGATACACGAC
GCAAAGAATCCATACCCGGCCCCCATCCTAAAATCTAGGGAATTGTTCCA
GAAACAGGGGGAGAGGAACTGCGTCCACTTGGAGCTGGGTATTGACGGGA
GTGGAATCACATATCAACACGGAGATCACGTCGGCGTGTGGCCAAGCAAT
CCAGAGGTTGAGGTTAACCGTCTACTATGCGCGCTGGGCCTTTGGGACAA
ACGTGACCATGTCATCGGTATCGAAAGTCTTGATCCAGCCCTTGCTAAGG
TACCATTCCCCGTTCCCACAACTTACTCCACTGTTTTAAGAAACTATATC
GACATATCAGCCGTTGCAGGAAGACAAATTTTGGGAAATCTGGCTCGTTT
TTCACCCTCCCCAGATGCTGAAGGGTTTATGAGAAGTTTGAATACGGATA
AAGAGCAATACGGTAGGATCATAGCAAACGGATGTCTGAAACTAGGGGAA
GTTTTGCAACTTGCGGCGGGAAACGACATAAAAGCAGTACCAACGTTAGA
AAATACTACTGCGTGGCCGATCCCATTCGATGTAATCGTTAGCGCCATTC
CTCGTTTACAACCTAGATATTTCAGCATAAGTAGTTCTCCCAAGCTACAT
CCTACAGCAATCCATGTTACTGCCGTAGTGCTAAAGTATCAGAGTGTCGC
TTCAGATAAGGTGCCACCGAAATGGGTGTACGGTGTTGGTTCAAACTTTA
TCCTTAACCTAAAATACGCCGCTTGCGGCGAAACAGCTCCGCTTATTGCA
CAGAATGGAAGCGCCGATCCTGCTCACACACCCTTTCCCCTATACGCTAT
AGAAGGTCCACGTGGGGCATACAAGCAGGAAATGATCTATAAAAGCCCAA
TCCATGTGAGAAGGAGTACATTTAGACTACCTACTAACCCTAAATCCCCT
GTAATCATGGTTGGACCAGGTACCGGAGTCGCGCCGTTTCGTGGATTCGT
TCAGGAACGTATCGCTCTAGCCCGTCGTACAATCGAGAAGAATGGTCCGG
ATGCACTAGCTGATTGGGGTAGGATATCTTTATTTTATGGGTGCAGAAAA
AGCAACGAGGACTTCCTATATAATGAAGAGTGGCCACAATACATCGACGA
ATTAAAAGGGAAGTTTACATTGCATACAGCTTTTTCAAGGGAGCCCCCGT
ATAAGCCGGATGGAAGTAAGATTTATGTGCAGGATCTATTATGGGATGAC
AGATCCAAGGTCGCAGACGCTATCATTAATGGAAAGGGCTACATCTATAT
ATGCGGTGATGCAAAGTCGATGGCAAAGTCAGTGGAGGACGTGCTGGCGA
AGATATTAGGGGAGGCTAAGGGCGGCACTATGGAAGTCGAAGGTGCGGCA
GAACTTAAATTATTAAAAGAAAGAAGTCGTCTTATGTTGGATGTTTGGTC
T

Seq. ID No: 175

>T4H_1ATGAAGACTAGGACTTCCAAACATCCTCCAGGCCCACGTGGGCT
GCCCCTGATTGGTAATCTACTAGACATGCCCGCATCATACGAGTGGTTAC
AATATAGGAAGTGGTCTGAGGAATTTAAGTCCGACATCATTTACCTGAAT
ATCTTAGGCACACAAATTGTAGTGACTAACACCCTTGAATCCACACTGGA
CTTACTTGAAAAGAGATCCTCTAAGTACTCCGGGAGGCATTCCTTTCAAC
TGCCGAATAACTGCGCTATGGGGTGGGCGTGGAATCTGGCTCTAATGTCT
TATGGGGACGAGTGGAGGGCCCACCGTAGGCTTGCCGCTAGAGGTTTCGA
TGCACAGGCGATGCCGAAATTTAACCACGCATTTACGCGTAATACTCGTG
GCCTGCTTAGGCGTCTTTTAGAGTCACCAGAGGCCTGGAATGAGCATGTA
AGGCACGAGGTCGGGTCAATGATAATTGAAATTACCTACGGGTTGGACGT
GCTTTCTAAGAATGATCCCTTTATTGAGTCCGCCGACAAAGGACTAGCGA
CATTGGCCTTAGCAGTAGTACCGGGTGCCTTTCTGGTTGACACTCTACCA
ATTCTTAAACACATACCTTCATGGTTTCCAGGGGCTGGCTTCAAACGTAA
GGCTAAGGAGTGGAAAAGATACGCCGATGAGGTTTTAGAAGCTCCTTATA
AGGCTCTAAAGGAAGAGATGGCAAGTGGAGCCGCGAAGCCTTCTTTTGTT
CAAAGGTGTCTGCAGGACATGGACCCAAATATCGATACAACGAACCAAGA
ACGTGTCATAAAGAACACAGCGGCAGAGATGTACGTGGCGGGCGCCGACA
CTTCTGCCAGCTTTATTGCAACGTTCGTGCTTGCTATGATACAGTACCCT
CAGGTACAGCGTAGAGCACAAGCAGAGTTGGACTCTGTCTTAGGCCCAGA
TAGACTGCCAACCTTCGGCGATATGCCTAGCCTTCCGTATTTGTCCGCGA
TAACTAAAGAGTGTTTCCGTTGGGAAGTAATCACCCCCATAAGTATTCCA
CACATGCTTACAGAGGACGACGAGTATCGTGGTTGGTTCCTACCGTCAGG
AACGGTTGTAATTCCAAATAGCTGGGCTATAATGAATGACCCGACAGTAT
ACCCAGACCCCTCCGTGTTCAATCCTGAAAGATTCCTGAAGGATGGTAAA
ATAGACCTTGAAGTGCAGGACCCGCAGTTAGCGGCTTTTGGTTATGGGCG
TAGAATTTGTCCCGGCATGAGGGTCGCCAATGCTTTTACGTGGTTAAGTG
CTGGATCAATCTTGGCCTCATTCAACATTTCAAAACCGGCAGCGAAAGAC
GGGACCCCCATCGAGCTTGATGTTAAGTATCGTAGCTCCAGTATTCGTCA
TCCAGAGGCCTTTGACTGTCTGTTTAAGCCCCGTTCAGAGAATACTAGAG
ACATGATCGTAAGCGCAGCCGCG

Seq. ID No: 176

>T4H_2ATGAGTAAGAGGTCAAAACACCCACCTGGCCCCAGAGGCTTGCC
GTTGATAGGGAACTTACTGGATATGCCTACGAATGATGAGTGGTTACAAT
ATCGTAAATGGAGTCAAGAGTTCAAGTCCGACATAATATACCTTAATGTT
TGCGGTACTCAAATCGTGGTTACGAATACCCTTGAGTCTACGCTTGATTT
GCTAGAAAGAAGATCTAGTAAGTATAGCGGCAGAATGGGTCTTGAATGGG
CTTTCATCTTGATGCCTTATGGAGATGAGTGGAGAGCGCACAGGAGACTA
GCAGCTAAAGGTTTCGACGCAAAGGCTATCCCAAAGTTTAATCCGACCTT
CACGAGAAACGCGCAGGATCTACTGAGAAGGCTTTTAGAATCTCCAGAGG
CTTGGCATGAGCACGTTCGTCATCAAGTCGGAGCGATGATAATAGAAGTA
TCTTACGGATTAGATGTATTGCACAAAAACGATCCCTTTATAGAGAGCGC
GGATAAAGCAGCCGTGACGTTCGCAATGGCCATCAAACCCGGAGCTTTCC
TTGTCAATACCGTGCCCATTTTAAAATACGTTCCTAGTTGGTTCCCCGGT
GCAGGATTTCAGAGAAAAGCTAAGGAGTGGAAAAGGTATAACGACGCAGT
GCTAGAAGCACCATTTAAGGCTTTGAAAGAAGAGATTACCAATGGGGCGG
CAAGACCATCTTTTGCCCAACAATGCCTGCAGAATATGGACCCGAACATT
GACACGGCATATCAGGAACGTGTGATAAAGGACACAAGTGCTGCGATGTA
TGGGGGAGGATCAGACACGTCAGTTTCATTTTTGGCTACATTCGTCCTGG
CTATGTTACAGTACCCCTCTGTCCAACGTCGGGCTCAGGTTGAGCTTGAC
TCCGTGCTAGGACGTGATCGTTTGCCAACATTCGACGACATGCCGGACCT
TCCCTATCTAGCAGCCGTTATGAAAGAGTGCCACAGATGGGAAATAGTTT
TGCCGCTTGCGATACCTCACATGCTTACCGCTGATGACGAGTACAGAGGA
TGGTTCCTTTTATCCGGCACGTTAGTTATCCCAAATAGCTGGGCAATACT
AAACGATCCTACGGTTTATCCCGATCCGTCTACTTTCAATCCTGAACGTT
TCTTAAAAGACGGGAAGATTGACCCCAATGTACAAGATCCCGAGCTTGCG
GCTTTTGGATACGGCAGAAGGACTTGTCCCGGCCGTAGGATAACCAACGC
TTTTACCTGGCTTTCCGCGGGATATATACTAGCCAGCTTCAATATAGAAA
ATGCAGTTGGTAATGATGGTATGCCCATCGAGCCCAAAGTTAAGTACCGT
TCTGAAACAATCAGACACCCAGATACTTTTGAGTGCGTCTTCACCCCAAG
GTCAGATGATACCAGAGATATGATCGGTAGCGCGTACACA

Seq. ID No: 177

>T4H_3ATGGGCAGGTGGCCCATAATTGGGAATCTATTAGACATGCCGCA
GAAATCCCCTTGGCTTACTTATGCCAAATGGAGTGAAGATTGCGACTCTG
ACATAATACACTTGAATGTCCTGGGTACGAGTATAGTCGTTTTATCTTCT
TTAGAGGCAATAAGTACGTTACTAGAGGGTAAGGCGGTCGATTTTAGTGA
TAGGCCTAAATCTACGATGATGTCCGAGCTTATGGGGTGGGAAAGGGGCT
TCGCGTTTATGCCCTATGGCCAGTTATGGAGATCCCACAGGAAAGCATTC
CATCAGGAGTTCAGCCCCCAGGTGGCGCACAGAAATCACCCGAAGTTAAT
TAAGGCGACACATAACTTACTGCGTCTGCTTCTGAACACCCCACAGCACT
GGCATGGGCATATTCGTAGACAAGCCGGGGCATCAATTATGGATATAGCA
TACGGTATTGAAGTACTACCTGAAAACGATCCGTACTTAGATATAGCAGA
GGCAGCCGTGAAGGCATTCAACGATGCCAGCGTGCCTGGCGCATTTCTAG
TCGATTCTATACCCCTACTTAAACATGTTCCGGCATGGGTCCCCGGGGCG
GGCTTTCAGCTTAAAGCAAAGGAGGGTAGACAGGCACTAGAAAACCTAAT
TGACTCACCTTACAACGCGATGAAAAAGGATCTTGCTGGCGGAAAAGCTA
AGAGCAGCTACACGTCTAGGTCCTTGGCAGCAATGGATGCGACTGGTGTG
ATTGAAGAAAATGAGACGATAATAAGGGAGACAGCGGCAATGGTGTATTT
AGGGGGAAGCGACTCCACCCCGAGCACGACATCCGTGTTCATACTGGCCA
TGTTAGCCCATCCCGAGGTGCAGAGAAAAGCTCATGCGGAGCTGGACAGC
GTGATCGGGAAAGCACAGCTTCCTACCTTTAAAGACCGTGGCTCACTACC
ATATGTTACGGCTGTGGCAAAAGAAGTCCTGAGGTGGGAACCTGTAGCGC
CGCTGGCGGTTCCTCGTAAGGTTCGTGTGGACTCCGAGTACAAGGGGTAC
AGGATACCCAAGGGCTCTATAGTCTTTCAAAACTCTTGGGCCCTTCTGCA
TGACGAGAAGACTTACCCTAACCCGTTAGCGTTCAACCCCGAGAGATTCC
TGAAAGACGGTCAATTAGACCCTAACGTCCAGGACCCAGATGTTGTGGCC
TTCGGGTATGGAAGGCGTTCTTGTCCGGGTAAAACGATGGGCTATGACTC
CGTATGGCTGAACGTGGCAAGTATTCTAGCCGCCTTCGACATTAAGAAGG
TTGCGAACCCGGATTCCACGAACGTGGAACCAAAGTTTGAACCGTTTGGA
ATAACCGTT

Seq. ID No: 178

>T4H_4ATGTATCTATTTAAGGCATACCTACGTCCTTCTCGTAGGTTACC
CCCAGGGCCAAGGGGGTGGCCCCTAATAGGAAACTTGCTAGACATGCCGA
CCTCCGACGAATGGGTAAGATACGCCCAATGGGTCCGTGAGTTTAAGTCT
GACGTTATCCACTTAGAAGTGTGTGGGACGCACATCGTTATTCTTAATAG
TGTAGAAAGCGCAGTTGATCTTTTAGAGAAGCGTTCATCCCTGTACTCAT
CTCGTCCGCCAACGCCTATGATGAGTGACCTGATGGGGTGGTCTTGGAAT
ACAGCTATGCTTCCGTACAACGACGAGTGGAGAGCGCAAAGGCGTCATTT
CCACGGTGAGTTCGACGGTAGAGCAATAGGAAAGCATTATCCTCCGATCA
TTAGAAGCACGCACGATTTGTTGCAAAGATTGTTGGACACCCCAGAGCAA
TGGCAGAGTCATATAAGGCACTTGGTCGGTGCAACGATATTGGATGTCGC
ATACGGAATCGAAGTTCTTCCAGCCGACGACCCATATGTACGTACAGCCG
AAGCTGCCTTTGCCTCCGTATCTGAGGCTATGGTGCCAGGCGCTTTCCTT
GTCGATGTACTGCCAATTTTGAAGCACATGCCGAGTTGGATGCCAGGGGC
AGGCTTCAAAAGAAAGGCAGTGGCGTGGAAGAAATTGGCAGACGCAGTTT
TTGATGCACCCTTCGCGGCGATGAAACAAGCAATGGCAGCGGGTACTGCG
AAGTCTTCATTTGGCAGTAGAAGTCTGAGGGATATAGACATTAAGGGAAA
CGTACAGAGCCAAGAGTTCTCTATCCAGGCCGCGGCTGGCACCATGTATA
ATGCCGGGTCAGACACGACAGTTGCGCTTTTAGAAACGTTCATGCTTGCT
ATGGTCCTACATCCCGAAGTCCAAACCAAGGCGCAAGCCGAAATGGACTT
AGTCCTAGGGAGAAGCAACTTGCCCACATTCGCTGACCAGGAGAGTTTGA
GCTACTTAGCAGCTGTAATGCAAGAAGTTTTTAGGTGGCAAGTCGTCGCG
CCCTTCGGCGTGCCGCACATGTCAACCGCAGACGATGAATACCGTGGTTA
TTTCATACCGGAAGGTACCATTGTAATTCCGAACGCCCATCAGATGCTGA
ACGATGAAGATGTTTATCCAGAGCCGTCCAAATTCAAACCCGAAAGATTT
TTAAAGGATGGCAAGCTAGACTTATCAGTGCGTAGCCCCCTAATTGCAGC
ATTCGGCTTCGGTAGAAGGATTTGTCCTGGCAGAGCCTTAGGAGAGAACT
CCGCCTGGTTGGCAGCTGGGTCCATCCTGACTATGTTCAATTTGTCTAAA
GCGACGGACCACAATGGTGTCACGATAGAACCCTCCGGCAGATATACATC
AGGTCTGGTTAGACACCCCGAGACGTTTAAATGTCAAATTACTCCCAGAT
CCAATGAGCCGAGAAGAGAACTGGCAGGGGAGATTGAATTGATCACGGGC
AGGATTCAAGAGTCTGAAGAAGCG

Seq. ID No: 179

>T4H_CPR_chimera_1TCCAGTAGCTCAAGCGGTTCAGTGGCCTACTT
TACGAAAGGCACTTATTGGGCAGTCCCCAAGGACCCATATGCATCCTCCT
ATGGCGCAGCTAATGGGGCGAAGGCGGGAAAGACAAGAGACATCATAGAG
AAAATGGAGGAGACCGGGAAAAACTGTGTAATCTTCTACGGCTCTCAAAC
CGGGACAGCTGAAGATTACGCTAGTAGATTGGCAAAAGAAGGGAGTCAAA
GGTTCGGACTAAAGACTATGGTCGCGGATTTAGAAGAATATGACTACGAG
AATCTGGACAAATGGCCAGAGGACAAAGTAGCATTCTTTGTACTTGCTAC
ATACGGCGAGGGGGAGCCTACGGATAACGCTGTACAATTACTGCAAAATT
TGCAGGATGAGAGCTTTGAGTTTTCTTCCGGTGAACGTAAGTTAAGTGGG
CTGAAATACGTTGTTTTTGGCCTTGGTAATAAAACATATGAGCACTACAA
CTTGATCGGCCGTACAGTTGATGCTCAACTAGCAAAGATGGGGGCGATCA
GAATAGGAGAAAGAGGGGAGGGCGATGACGATAAGTCTATGGAGGAAGAT
TATCTTGAGTGGAAGGACGGTATGTGGGAAGCGTTTGCCACGGCGATGGG
AGTTGAAGAAGGCCAAGGAGGTGATTCCGCCGACTTTGTTGTTAGCGAGC
TGGAATCACATCCACCTGAGAAAGTCTATCAAGGCGAATTTAGCGCGAGA
GCCTTAACTAAAACAAAGGGGATTCACGATGCGAAAAATCCGTTTGCTGC
ACCGATCGCAGTTGCACGTGAGTTGTTTCAAAGCGTAGTGGACCGTAATT
GTGTCCACGTAGAGTTCAATATCGAGGGAAGCGGGATCACGTATCAACAC
GGGGACCATGTCGGTCTATGGCCTCTGAATCCGGATGTAGAGGTAGAAAG
ATTATTATGCGTCCTAGGTCTAGCCGAAAAGAGAGACGCAGTTATAAGCA
TTGAGAGCCTAGACCCAGCTTTAGCTAAGGTCCCCTTTCCAGTACCTACC
ACCTACGGTGCAGTTCTACGTCACTACATCGACATATCCGCCGTTGCTGG
CCGTCAGATCCTTGGCACTTTATCAAAATTCGCGCCGACTCCGGAAGCCG
AAGCCTTTCTAAGGAATCTAAATACCAACAAAGAAGAGTACCATAATGTC
GTAGCCAACGGCTGCTTGAAGTTGGGTGAGATCCTGCAGATCGCGACTGG
CAATGACATAACCGTTCCCCCGACCACGGCCAACACGACAAAGTGGCCCA
TACCTTTTGACATTATAGTAAGCGCGATACCAAGGTTGCAGCCAAGATAT
TATTCCATTAGTAGTTCCCCGAAAATCCACCCTAACACGATACATGCAAC
TGTCGTCGTATTAAAGTACGAAAACGTGCCGACAGAGCCCATTCCCAGGA
AGTGGGTTTATGGGGTGGGGTCCAACTTTCTATTAAACCTGAAGTATGCT
GTGAACAAGGAGCCAGTACCCTACATCACGCAGAATGGTGAACAGCGTGT
CGGAGTCCCAGAATATCTAATAGCGGGCCCTAGGGGGAGTTACAAAACAG
AGTCCTTCTACAAGGCCCCCATACATGTGAGAAGATCTACCTTCAGGCTA
CCCACCAATCCGAAAAGTCCAGTTATCATGATTGGACCGGGTACGGGCGT
TGCACCGTTTCGTGGCTTCGTACAGGAGAGGGTCGCACTGGCTCGTCGTT
CCATTGAAAAGAATGGGCCGGACAGCCTGGCTGACTGGGGACGTATTTCC
TTGTTTTACGGTTGCAGACGTAGCGATGAAGATTTCCTTTACAAAGATGA
GTGGCCACAGTATGAGGCCGAGCTTAAAGGCAAGTTCAAGCTACACTGTG
CTTTCAGCCGTCAGAACTATAAACCCGATGGCAGTAAAATTTATGTGCAA
GACCTGATATGGGAAGATCGTGAGCATATCGCAGATGCGATACTTAACGG
TAAGGGTTATGTATACATCTGTGGAGAAGCAAAGTCAATGAGCAAACAGG
TAGAAGAGGTTCTGGCCAAAATTCTGGGTGAAGCCAAAGGGGGCTCTGGT
CCGGTAGAAGGAGTGGCCGAAGTCAAACTGTTGAAGGAGCGTAGCAGACT
AATGCTAGACGTCTGGAGC

Seq. ID No: 180

>T4H_CPR_chimera_2TCTTCATCATCCAGTTCAGGGACGATAGCTTA
TTTTACCAAAGGAACCTACTGGGGTATTGTCAAAGATCCATATGCTCCTA
ACTATCCTCCTGCAAATGGGAATAAGCCCGCCAAAACAAGAAATATTGTT
GAGAAAATGGACGAAAGCAACAAGAATTGTGTTGTATTCTATGGTTCCCA
GACTGGAACTGCCGAAGATTACGCGAGTCGTTTAGCCAAGGAAGGGAAAT
CACGTTTCGGGCTGGAAACGATGGTAGCTGATCTTGAGGACTATGACTTC
GATAACTTGGACACACTAGGAGATGACAAAGTTGCAATTTTTGTGTTGGC
AACCTATGGTGAGGGGGAGCCCACAGATAACGCGGTTCAATTGCTACAGA
ATTTACAAGACGAGAGCTTTGAGTTTTCATCTGGCGAGAGGAAGCTAAGT
GGATTGAAATATGTGGTCTTTGGACTAGGAAACAAGACATATGAACACTA
CAATTTGATAGGAAGAACCGTTGATGCTCAGCTTGCTAAGATGGGTGCTA
TACGTATCGGAGAAAGAGGGGAAGGAGACGACGACAAATCTATGGAGGAG
GACTATCTTGAATGGAAAGATGGGATGTGGGAGGCATTTGCCACGGCAAT
GGGGGTGGAAGAAGGCCAGGGTGGTGATTCCGCTGACTTTGTTGTGAGCG
AATTGGAAAGTCATCCTCCTGAGAAAGTTTATCAAGGGGAATTTAGCGCG
CGTGCTCTGACAAAGACTAAAGGAATACATGACGCAAAGAACCCCTTCGC
CGCTCCCATAGCGGTCGCCAGGGAGCTGTTCCAGTCTGTCGTTGACAGAA
ATTGCGTGCACGTAGAGTTCAACATTGAAGGTAGTGGTATTACATACCAA
CATGGTGATCATGTCGGACTTTGGCCTCTTAACCCGGATGTAGAAGTTGA
ACGTCTGCTTTGTGTGTTAGGCCTGGCCGAAAAACGTGACGCCGTTATAA
GCATTGAGTCTCTGGACCCTGCATTAGCAAAGGTCCCGTTCCCTGTTCCT
ACGACATACGGTGCTGTCCTTCGTCATTACATTGATATCAGTGCTGTAGC
AGGAAGGCAAATTCTAGGGACCCTAAGTAAGTTTGCACCAACCCCGGAAG
CAGAGGCATTTTTACGTAACTTAAATACCAATAAAGAAGAATACCACAAC
GTAGTAGCGAACGGATGTCTAAAACTTGGAGAGATTTTACAAATTGCTAC
CGGCAACGACATTACGGTACCGCCGACCACGGCCAATACAACTAAGTGGC
CGATACCATTTGACATCATAGTGTCAGCCATACCGCGTTTGCAACCACGT
TATTACTCTATCTCAAGTAGCCCAAAGATCCACCCCAACACAATTCATGC
AACCGTGGTGGTTTTGAAGTATGAGAACGTCCCAACGGAGCCTATCCCCA
GGAAGTGGGTATACGGAGTGGGTTCTAACTTTTTGTTAAACTTAAAATAC
GCAGTAAATAAAGAGCCTGTCCCGTACATAACACAGAATGGGGAACAGCG
TGTTGGCGTGCCTGAGTATTTGATCGCTGGACCCAGAGGCTCTTATAAAA
CTGAAAGTTTTTACAAGGCCCCGATCCACGTGAGGAGGAGCACCTTCAGA
TTACCCACCAATCCTAAGTCCCCAGTCATAATGATCGGGCCTGGAACAGG
CGTGGCCCCCTTTAGGGGCTTTGTTCAAGAGAGAGTAGCATTGGCGCGTC
GTTCCATAGAAAAGAACGGCCCAGATTCACTGGCCGACTGGGGCAGGATT
AGTTTGTTTTACGGGTGCCGTCGTTCTGATGAAGATTTCTTGTACAAAGA
TGAGTGGCCGCAGTACGAAGCGGAATTGAAAGGCAAATTTAAATTACACT
GCGCCTTTAGTCGTCAGAACTACAAGCCGGACGGCAGCAAGATTTATGTC
CAGGATCTTATATGGGAGGACAGAGAACACATCGCAGATGCGATACTGAA
TGGTAAGGGCTACGTTTACATATGTGGAGAAGCCAAATCCATGTCTAAAC
AGGTTGAAGAGGTCCTAGCCAAAATACTAGGGGAGGCAAAAGGGGGTAGT
GGCCCCGTAGAGGGGGTGGCTGAAGTTAAGCTGTTAAAGGAGAGGAGTAG
ACTAATGTTAGATGTGTGGAGC

Seq. ID No: 181

>T5H-CPR_1AGCAGTAGCTCCTCCTCAGGCGGCTTACTTGCCTTTTTAT
ACTTATTTAGGGGTACGTTATTTGCATCAGGAAAAGCGAGCGATGCCGGG
TCAAAATTAGCAGGAGGGTCTGATCTGGACAGCAGTGCTGACGCCGCTGC
CAACGACTTTGTTACTAAACTGACGTCCCAAAATAAACGTATAGCTATTT
TCTACGGAAGCCAGACGGGAACAGCAGAAGAGTATGCGACAAAGATCGCT
AAAGAAGCGAAAGCCCGTTTTGGAACGTCATCCCTTGTGTGTGACCCCGA
GGAGTATGAATTTGAAAAATTAGACCAGTTGCCTTCCGATTGTGTGGCCT
GTTTTGTCATGGCGACCTACGGAGAGGGCGAACCGACCGACAACGCAGTG
GGTCTTATGGAGTTCTTAGATGGAGAAGATGTGCAGTTCAGCAATGGGTC
TAGCCTGGATAATCTTAACTACGTTATCTTTGGCCTAGGAAATAGAACTT
ACGAGCACTATAACGCAATCGCACGTAAACTAGACGCCCGTCTTGAATCT
CTAGGGGCGAAGAGGATAGGCGAGAGGGGCGAGGGAGATGACGATAAGAG
TATGGAGGAGGATTATTTAGCTTGGAAAGATGGAATGTTTGAAGCCTTGG
CAAGCTCATTAGGTTTCGAAGAGGGCGGAGGGGGCGACGTGGCCGATTTT
AAGGTACGTGAGGTAGCAGATCACCCGGAAGATAAAGTGTATAGGGGTGA
ACTTTCTGCGAGAGCCTTGCTAGGCACGAAGGGCATTCATGATGCCAAGA
ACCCCTACAACGCAGTAGTAAAGGAGGCTAGGGAACTATTCGTCGAAGGG
ACAGCTGACAGGACGTGTGTCCATGTTGAGTTCGACATCGAGGGTTCCGG
AATCTCCTACCAACACGGTGATCATATAGCCGTATGGGCTCATAATCCTG
AACAGGAAGTTGAAAGAGCATTAGCCGTCCTTGGTTTGCTGGGCAAACGT
GATACGGTGATAGATGTAGAAAGTCTCGATCCCACCTTGGCGAAGGTTCC
TTTTCCGGTTCCAACTACGTACGAGGCCGTCTTTAGACATTACCTGGATA
TTTGTGCACACGCGAGCAGACAAACTCTGAACAACTTCGCAAAGTATGCG
CCCACCCCCGAAGCCCGTGCTAAGCTGGAGAAAGCCTGCGGAGACAAGGC
AGCGTTCCAAGAGGCAATAGGCCATAGATGTCTGAAAACTTTTGAGGCAT
TACAATTGATTGTCGGCGATGATCTAGGTGGTGACTCCGTTGCAAAAGCT
ACTGCCTGGGAGATACCATTTGATAGAGTAATCTCTGACCTTCCCAGAGT
CGGACCAAGGTTTTATTCAATATCATCTTCACCTAAGATGCACCCCAAGA
CGGTGCACATCACGGCTGTCGTGTTGAGATATAGGCCAGAGGCAGCAGGG
CAAGACTCCCCGTACGTTCATGGCTTAGCAACGAACTTTATATCCGCTAT
AAAGATGGCTAAAAATAATGAGCAGCCTAGTGGACCGGATGATCCGAGAT
TCGGAACTCCTGGGTATGACCTAGCAGGCCCCAGGGGTGCCTACACAAAA
GAGAGTCTATTTAGGGCCCCTATCCATATAAGACGTAGTAATTTCCGTCT
ACCCACGTCACCCAAAATCCCAGTAATCATGGTAGGTCCTGGCACGGGGG
TCGCACCCTTTCGTTCATTCGTGCAGGAGCGTGTCTGCAGCGCACAGAAG
ACCTGTGATAAAGTAAATCAATCCCCGGCCGAGGCCCTTCAGGACTGGGG
TAATATCTGGCTATTCTATGGTTGTAGAAGGAGCAACGAAGACTTCCTGT
ATAAGGATGAGTGGCCCGAGTACGCGAGCAAGCTGGGAGGGAAATTCCAA
ATGGAGACAGCCGTCAGTAGAGAGAAGTTCAAGCCAGACGGCAGTAAACT
TTATGTGCAGGACTTAATATGGGAGAGGCGTAAAGAACTGGCACAAGATA
TACTAGATAAGAAAGCGTATATTTACATCTGTGGTGAGGCGAAAGGGATG
GCCCACGATGTCGAAGAAATGTTTGGTAGGGTGCTTGAGGAGGCTAAAGG
TTCCGCTGAAGCTGGCAGACGTGAACTTAAATTACTGAAGGAGAGGTCCC
GTTTACTTTTAGACGTTTGGTCT

Seq. ID No: 182

>T5H-CPR_2TCCTCCAGCTCTTCATCACTGTTCTCAACTACTGACGTGA
TTCTATTCAGCCTTATTGTGGGTGTCATGACATATTGGTTTCTGTTTCGT
AAGAAGAAGGAGGAAGTGCCAGAATTTACAAAAATACAGACAACAACGAG
TTCAGTGAAAGATCGTTCTTTCGTCGAAAAGATGAAGAAGACAGGTCGTA
ATATCATTGTGTTTTATGGAAGTCAGACCGGGACCGCAGAGGAGTTTGCC
AACCGTCTGTCCAAGGACGCGCACAGATATGGAATGCGTGGTATGGCTGC
CGATCCTGAAGAGTACGATCTAGCGGACCTGTCATCACTGCCTGAGATAG
AAAAAGCCTTGGCAATCTTTTGTATGGCGACTTATGGTGAGGGCGACCCC
ACGGACAATGCCCAGGACTTTTACGACTGGCTTCAGGAGACAGACGTCGA
TCTTAGCGGCGTTAAATATGCAGTCTTCGCCTTGGGAAACAAGACATACG
AGCATTTCAACGCGATGGGAAAATACGTAGACAAAAGGCTAGAGCAACTT
GGGGCACAGAGAATATTTGATCTTGGATTGGGAGATGATGACGGAAATTT
GGAGGAAGACTTCATTACATGGAGGGAGCAGTTTTGGCCGGCTGTGTGCG
AGCACTTTGGGGTCGAAGCCACCGGTGAGGAAAGCAGTATAAGGCAGTAT
GAACTTATGGTGCATACCGATATGGACATGGCCAAAGTATATACGGGAGA
GATGGGTCGTCTAAAGAGTTATGAAAATCAGAAGCCGCCCTTTGATGCCA
AGAACCCTTTCCTGGCCGTTGTCACGACCAATAGAAAGCTAAATCAAGGC
ACGGAGAGGCACCTTATGCACCTGGAGCTTGATATTTCAGATAGCAAGAT
ACGTTACGAAAGCGGGGACCATGTCGCCGTCTATCCTGCAAACGACTCCG
CACTGGTTAACCAGTTAGGAGAGATATTGGGCGCTGATCTTGATATAATA
ATGTCCCTAAACAACCTGGACGAAGAAAGCAACAAAAAGCATCCGTTTCC
TTGCCCAACCAGCTACAGGACTGCGTTAACGTATTATTTGGATATCACTA
ACCCTCCTAGAACCAACGTGTTATACGAATTAGCACAATACGCGAGCGAG
CCGACCGAACACGAGCAACTGAGGAAAATGGCTAGTAGCTCAGGGGAAGG
AAAGGAGTTATATCTGAGATGGGTCCTAGAGGCAAGGCGTCATATCTTAG
CCATTCTACAGGATTATCCGTCCCTTAGGCCTCCCATAGACCATTTATGT
GAGTTATTGCCCAGATTGCAAGCGAGATATTACTCCATCGCAAGTTCATC
AAAAGTTCATCCTAATTCTGTGCACATCTGTGCGGTAGCGGTGGAATATG
AAACTAAAACGGGGAGGATTAATAAGGGTGTCGCTACGAGCTGGCTAAGG
GCTAAAGAGCCGGCTGGCGAAAATGGCGGACGTGCATTGGTCCCAATGTA
CGTAAGAAAGTCCCAGTTCAGACTACCTTTCAAGGCGACGACGCCTGTCA
TAATGGTAGGCCCAGGTACCGGTGTGGCTCCATTTATCGGGTTCATTCAG
GAAAGAGCGTGGCTGAGGCAACAGGGAAAGGAGGTCGGTGAGACACTATT
GTACTACGGCTGCAGGAGGAGTGACGAGGATTACCTTTACAGGGAAGAGT
TGGCAGGCTTTCATAAAGATGGTGCCCTTACACAGCTTAACGTCGCCTTC
AGTAGAGAACAGCCGCAAAAGGTTTATGTTCAACACTTACTTAAAAAGGA
CAAAGAACACTTGTGGAAACTAATACATGAGGGTGGGGCGCATATTTATG
TGTGTGGAGATGCGAGGAATATGGCTCGTGATGTGCAAAATACGTTTTAT
GACATAGTGGCTGAGCAGGGGGCGATGGAACATGCCCAAGCCGTGGATTA
CGTCAAGAAGTTAATGACCAAAGGCCGTTACAGCCTAGACGTTTGGTCT

Seq. ID No: 183

>T5H-CPR_3AGTAGCTCCTCATCATCCGAGGCAGTAGCCGAGGAGGTTT
CCCTGTTTTCTATGACTGATATGATCCTATTCTCCTTGATTGTTGGTTTG
TTGACGTACTGGTTTCTTTTTCGTAAGAAGAAAGAAGAGGTTCCGGAATT
TACTAAGATTCAAACTTTGACCAGTAGCGTCCGTGAGTCATCTTTCGTCG
AAAAGATGAAAAAGACAGGTAGAAATATCATAGTATTCTACGGCAGTCAA
ACCGGTACAGCGGAGGAGTTCGCTAACAGATTATCTAAGGATGCCCACCG
TTATGGCATGAGAGGAATGTCCGCTGATCCTGAGGAGTACGATCTGGCTG
ACCTAAGCTCCCTACCCGAAATTGATAATGCACTGGTGGTTTTCTGTATG
GCTACATACGGGGAGGGAGACCCGACGGACAACGCTCAAGACTTCTACGA
CTGGCTGCAGGAAACAGACGTTGACCTGTCTGGTGTAAAATTCGCGGTGT
TTGGCCTGGGGAATAAGACTTATGAGCACTTCAACGCGATGGGAAAATAT
GTTGATAAAAGACTTGAGCAACTTGGAGCACAGAGGATTTTCGAACTTGG
ATTGGGAGATGACGACGGTAACTTAGAGGAGGATTTTATCACCTGGCGTG
AACAATTCTGGCCCGCAGTGTGTGAACATTTCGGGGTGGAGGCGACCGGT
GAGGAAAGCTCAATCAGGCAATACGAGTTGGTGGTTCATACCGATATAGA
CGCCGCCAAAGTTTACATGGGAGAGATGGGCAGACTGAAGTCTTACGAGA
ATCAAAAGCCCCCTTTCGACGCCAAGAACCCATTTTTAGCGGCAGTCACT
ACTAATAGGAAACTGAACCAAGGCACCGAACGTCACCTTATGCATCTGGA
ACTGGATATCAGCGATAGCAAGATCAGGTATGAATCAGGAGATCATGTGG
CGGTCTATCCTGCCAACGATTCAGCTCTTGTCAACCAGTTGGGAAAAATT
TTGGGTGCTGACTTAGACGTTGTAATGTCACTTAACAATTTAGATGAGGA
GAGCAACAAAAAGCACCCTTTCCCCTGCCCCACAAGCTACAGGACCGCAT
TAACTTATTACCTAGACATAACGAACCCTCCACGTACTAACGTCTTATAT
GAATTGGCACAATACGCCTCCGAACCGTCCGAACAGGAGTTACTGCGTAA
GATGGCTAGTAGTTCAGGAGAAGGTAAGGAATTATACCTAAGTTGGGTTG
TAGAGGCAAGGAGACACATCCTTGCCATATTGCAAGATTGTCCATCTCTG
AGGCCACCAATCGACCACCTTTGCGAGTTGCTACCTAGACTTCAGGCGAG
GTACTATAGTATAGCATCTTCCAGCAAAGTGCATCCCAATTCAGTGCATA
TTTGCGCCGTTGTCGTTGAATACGAAACAAAGGCAGGAAGGATCAATAAG
GGCGTCGCGACAAATTGGCTGCGTGCGAAGGAGCCTGCGGGGGAGAACGG
TGGACGTGCTCTAGTTCCGATGTTTGTTAGAAAATCTCAATTCCGTTTGC
CTTTTAAAGCTACCACCCCCGTAATAATGGTAGGTCCAGGAACCGGTGTA
GCACCATTTATTGGTTTCATACAAGAAAGAGCTTGGCTAAGGCAGCAAGG
AAAGGAAGTAGGCGAGACCTTGTTATATTATGGTTGTCGTCGTTCAGATG
AGGATTACCTGTACAGAGAGGAGCTGGCTCAATTCCATCGTGATGGGGCT
CTGACCCAATTAAATGTGGCATTCAGCCGTGAACAATCTCACAAAGTGTA
TGTTCAACATTTGCTAAAACAGGATAGAGAACATCTATGGAAATTAATAG
AAGGGGGTGCTCACATATATGTCTGCGGGGATGCCAGAAATATGGCGCGT
GACGTGCAGAACACGTTTTATGACATTGTGGCCGAGCTAGGCGCGATGGA
GCACGCCCAAGCCGTCGATTACATCAAAAAGTTGATGACGAAGGGCAGGT
ACTCATTAGATGTGTGGAGT

Seq. ID No: 184

>T5H-CPR_4TCCTCTAGCAGTAGCTCTGCCGCGGCTGCAGACGGCGACG
GTGGGCAATCTCGTAGACTTCTGGCTTTACTGGCTACCAGTCTAGCAGTA
TTAGTGGGATGCGGCGTTGCGTTATTGTTCAGGAGAAGTAGTAGTGGTGC
AGCTCCACTAGCTAGGCAGGCCGCTGCAGCCAAACCCCTTGCAGCTAAAA
AGGACCAGGAACCAGATCCAGATGACGGCAGGCAAAGGGTCGCCTTGTTC
TTCGGCACCCAGACCGGGACAGCGGAGGGTTTTGCGAAGGCACTAGCCGA
GGAAGCCAAGGCGAGGTACGATAAAGCAGTGTTTAAAGTCTTGGATCTGG
ACGATTACGCAGCCGAGGATGAAGAATATGAAGAAAAGTTGAAAAAGGAG
AACATCGCGTTTTTCTTTTTGGCAACGTATGGGGATGGAGAACCAACAGA
CAACGCAGCGAGATTTTACAAGTGGTTCAGCGAAGGGAATGAAAGGGGTG
AGTGGTTGTCTAACCTGCAGTACGGGGTATTTGCATTGGGGAATAGACAG
TACGAACATTTTAATAAGGTCGGAAAGGAAGTGGACCAACTACTGGCAGA
ACAGGGCGGGAAAAGAATTGTACCTGTTGGATTGGGTGATGACGACCAAT
GTATAGAAGATGACTTCAACGCGTGGAAAGAGTTACTGTGGCCTGAACTG
GATAAACTTTTGAGGGTAGAAGACAACTCATCCGCAGCTCAAAGTCCCTA
TACTGCGGCGATTCCTCAATATAGAATAGTGCTAACAAAACCCGAAGACG
CAACACATATTAATAAGAGCTTTAGTCTGTCAAATGGACACGTGGTGTAC
GACTCACAACACCCGTGCAGGGCCAACGTGGCGGTAAGACGTGAGTTACA
CACGCCGGCAAGTGACAGGAGCTGCATTCATCTTGAGTTCGACATCGCCG
GGACCTCACTTACGTACGAGACGGGAGATCACGTTGGAGTATATGCCGAG
AACAGTACTGAGACGGTGGAGGAGGCGGAAAAACTACTTGATTACAGCCC
AGATACTTATTTCAGCATTTATGCAGATCAAGAGGACGGTACCCCATTGT
TTGGTGGTAGTCTGCCACCCCCGTTTCCGCCATGTACAGTAAGGGTGGCT
TTAGCAAGATATGCTGACCTATTAAATTCCCCCAAGAAGAGTGTATTATT
AGCCCTGGCCGCTCATGCATCCGACCCCAAGGAAGCTGAGAGGCTGCGTC
ACCTGGCATCTCCGGCTGGAAAGAAGGAGTATTCTCAGTGGATTATAGCG
TCTCAAAGGTCTCTGTTAGAGGTAATATCAGAGTTTCCCTCCGCTAAGCC
ACCACTGGGAGTTTTCTTCGCAGCGATTGCCCCGAGGTTACAACCGCGTT
ATTACTCCATTAGCAGCAGTCCCAGGATGGCGCCCACAAGAATACATGTC
ACCTGTTCATTGGTGCACGGCCAATCTCCCACCGGTAGAATCCACAAAGG
CGTATGCAGTACATGGATGAAAAACAGTACGCCCAGCGAGGAAGAGTCCG
AGGAGTGTTCCTGGGCACCAATCTTTGTGAGACAATCCAATTTCAAGCTA
CCCGCCGATCCCACTGTCCCGATCATTATGGTGGGGCCTGGAACAGGTCT
AGCTCCATTCAGGGGTTTTCTACAGGAAAGACTGGCTTTAAAAGAAACGG
GAGTTGAGTTGGGGCGTGCAATTCTATTTTTCGGATGTCGTAATCGTCAA
ATGGATTTTATTTACGAGGATGAGCTGAACAATTTTACGGAAAGTGGCGC
GCTTTCCGAGCTGGTAGTTGCCTTTTCTCGTGAAGGTCCCACCAAGGAAT
ATGTTCAACACAAGATGGCAGAAAAAGCGGCAGATTTATGGTCAATAGTA
TCTCAAGGGGGTTATGTCTATGTGTGCGGGGACGCTAAGGGAATGGCACG
TGACGTGCACAGAGCATTGCACACCATAGTACAGGAACAAGTCACACAAA
GGACCTCCAACTTTGGGCTTTGGAAGTTCCGTCTTGTTTCACTAAAT

Seq. ID No: 185

>T5H-CPR_5AGTTCATCTTCATCATCTGCGGCAGCCGCAGGCGGCGATC
CACTAGCCGCGCTGGCTGCCACCGCCGCAGCGTTAGTGGCAGGAGTAGTA
ATACTAGCTGTTTGGTTCAGGTCAGGTGGAGGTGCGCCTCCGAAAGCAGC
TGCTCCACCACCTCGTCCACCTCCGGTTAAGATTGAAGCCGACGCAGATG
CTGATGATGGCAGGAAGAGGGTGACCGTGTTCTTTGGCACTCAGACCGGG
ACCGCAGAAGGCTTTGCCAAAGCGATGGCCGAAGAAGCTAGGGCCCGTTA
CGAAAAGGCTGTGTTCAAAGTCGTTGACCTGGATGACTATGCGGCAGAGG
ATGAGGAATATGAGGAAAAACTTCGTAAAGAAACCATCGTCTTACTGTTC
CTGGCGACGTATGGGGACGGTGAGCCTACCGACAATGCGGCGCGTTTCTA
TAAGTGGTTTACAGAAGGTAAAGAAAAAGAGGTTTGGCTAAAAGACCTAA
AATATGCGGTATTTGGATTGGGTAACAGACAATACGAGCACTTTAATAAG
GTCGCCAAGGTAGTCGATGAGCTATTGGAGGAACAAGGAGGGAAAAGACT
TGTTCCCGTGGGTTTAGGAGATGATGACCAGTGCATAGAGGATGATTTCA
CGGCTTGGAAGGAGCAGGTCTGGCCAGAACTTGATCAGCTTTTAAGAGAC
GAAGACGACACAACGGGTGCATCTACACCTTACACTGCGGCCATACCCGA
ATATCGTATCGTTTTCATTGATAAGAGCGATGTATCATTTCAAGACAAGA
GCTGGTCCCTTGCAAACGGTTCAGGGGTCATAGATATTCATCATCCTGTA
AGGAGTAACGTCGCAGTCCGTAAAGAGTTGCACAAGCCGGCTTCTGATAG
GTCTTGTATCCACTTGGAGTTCGACATTTCAGGCACCGGCCTTGTGTATG
AGACCGGCGATCATGTCGGGGTGTATAGCGAGAATGCCATCGAAACAGTT
GAGCAAGCGGAAAAGCTACTAGACCTTTCTCCAGATACGTTCTTTAGCGT
CCATGCAGATGCAGAGGACGGATCTCCCAGAAAAGGAGGGGGATCTTTAG
CCCCACCCTTCCCGTCTCCATGTACTCTTAGAACCGCCCTATTGAGGTAT
GCTGACCTGCTGAACAGTCCTAAGAAAGCTGCATTAGTAGCTCTGGCAGC
GCATGCGTCCGATCTAGCTGAAGCAGAAAGGCTACGTTTCTTGGCTTCCC
CAGCCGGAAAAGATGAGTATTCACAATGGGTGGTAGCGTCCCAGAGGTCC
CTGTTGGAAGTTATGGCCGCGTTTCCCAGTGCAAAGCCGCCGCTGGGAGT
GTTCTTTGCAGCTGTCGCCCCCAGACTTCAACCACGTTACTACTCCATTT
CATCCTCTCCGAAAATGGCTCCCTCCCGTATCCACGTCACCTGCGCGCTA
GTGTACGGCCCGACTCCTACAGGCCGTATACACCAGGGCGTTTGTAGCAC
GTGGATGAAGAATGCAATACCAAGCGAGTACAGTGAGGAGTGTTCCTGGG
CACCCATATACGTTAGACAAAGTAATTTTAAACTACCCGCGGACCCGACG
ACCCCAATTATTATGATAGGACCCGGTACAGGGCTTGCTCCATTTCGTGG
CTTTCTACAAGAACGTTTGGCTTTAAAACAGTCTGGAGTTGAGCTGGGGA
ACTCTGTGTTATTTTTCGGGTGTAGAAACCGTAATATGGATTATATTTAC
GAAGATGAACTACAGAACTTTATTCAGGAGGGCGCTTTGAGCGAGTTGAT
CGTCGCTTTTAGTAGGGAAGGCCCAGCGAAGGAATATGTCCAACACAAAA
TGACTGAAAAGGCAACGGAAATCTGGAATATAGTCTCTCAAGGTGGTTAT
ATTTACGTTTGCGGGGATGCAAAGGGCATGGCTAGGGACGTCCACCGTGC
TTTGCACACTATAGTACAAGAACAAGGCTCTCTTGATAGCAGTAAGACAG
AGTCATATGTAAAATCTCTTCAAATGGACGGCCGTTATTTACGTGACGTC
TGG

Seq. ID No: 186

>T5H-CPR_6TCTTCCTCCTCCTCAAGCGCCGCTGCATATCTGTTTAGGG
ACCAAATTTTTCGTTCTAGCAGTCCGAAAGTAGTAGTACCCGCCCCTTCC
AAACTGGCGAACGGCCACGGAAATCCTCGTAACTTTGTTAGTAAGATGAA
GGAAGGGAAGAAGAGGATTGTAATCTTTTATGGGAGCCAGACTGGTACCG
CGGAGGAGTATGCGATTAGGATCGCTAAAGAAGCTAAAACGAAGTTCGGT
CTTACCTCACTGGTTTGCGACCCTGAGGAATACGATTTCGAGAACCTGGA
CCAAGTACCAGAAGACTGCTGCGTATTCTTTGTGATGGCCACATACGGAG
AGGGGGAACCGACGGACAATGCTGTTCAACTGATGCAAAACTTAGAGGAT
GAGTCCTTCGAGTTTAGTAATGGATCACACAGGTTGGACGGTTTGAAATA
TGTGGTGTTTGCGCTGGGCAACAAAACGTACGAGCACTATAACGCTATTG
GACGTAAGGTAGACACATTGTTAACAGACATGGGGGCGACAAAAATCGGG
GAACGTGGAGAAGGAGACGACGATAAGTCAATGGAAGAGGATTATTTAGA
GTGGAAAGATGGAATGTGGAAGGCGTTTTCTGAAGCGATGGGTGTAGAGG
AAGGGCAGGGCGGGGATACTCCAGATTTTGCTGTTACTGAGCTTGATAGT
CACCCGCCAGAGAAAGTATATCTAGGCGAGCTTAGTGCCAGGGCCTTAAC
TAGGACTAAAGGCATTTATGACGGTAAAAATCCATACCCCTCCGCCGTAA
AACATAGTAGGGAACTTTTTCAGGCTGGTGCAGAGAGGAACTGCGTACAC
GCGGAACTAGATATTGAGGGCTCTGGCATTACATATCAGCACGGTGATCA
TGTAGGAGTGTGGCCGAGCAACCCCGATGTTGAGGTCGATCGTATGCTAT
ACGTGTTAGGTCTATATGGCAAGAAAGACGCCGTGATAAATATAGATTCC
CTAGACCCTGCGCTGGCGAAAGTACCCTTCCCCGTACCGACTACATATGC
CACGGTTCTGAGACACTACATCGACATATGTGCTGTGGCTGGGCGTCAGA
TGTTGGGGGTCCTTAGCAAGTTCGCACCGCATCCGAAAGCCGAGGCTTTC
CTGAAATCATTGAATAGCGATAAGGAAGAGTACTCAAACATAGTAACGAA
CGGGTGTTTTAAACTGGGTGAAGTTCTGCAGTTAGCGGCAGGCGACGATA
TTAAACTATGTCCCACCCCAGACAACACCACAGCTTGGGCAATACCCTTC
GATATCATAGTATCCTCTATACCGAGACTACAACCGCGTTTCTACAGTAT
TAGTTCCTCTCCCAAATTATACCCCAATGCAATCCATCTAACAGCTGTAG
TGCTGAAGTACGATAGTATCCCAAATAGGCTGGTGGAGTCTCGTTTTGTA
TATGGCGTGGCCACAAATTTTCTATTGAATGTGAAGTACGCAGCTAATGG
TGAGACGGCTCCATTCATCGCCGAACCAGTAATATCTGAGCCGGCACATG
TCTCACTTCCAAAATACGCCATAGAAGGACCTAGAGGAGCCCATATCGAG
GACAATATTTATAAGATACCGATACATGTCCGTAGATCCACTTTTAGGCT
ACCTGCTAATCCGAAGATTCCAGTAATCATGGTCGGACCGGGAACAGGCG
TCGCGCCCTTTAGAGGGTTCGTGCAGGAGAGAGTTGCACTTGCTAAACGT
AGCATTGAGAAAAATGGGCCAGACGCCCTTGCCGATTGGGGCAGCATTAC
ACTGTTTTACGGTTGTAGGAAATCCAATGAAGACTTTTTATATAAGGAAG
AGTGGCCTCAATATGCGGAAGAGTTAAAAGGTAAATTTAAGATGCATTGC
GCTTTCAGTAGAGAGCCTCCTTACAAGCCTGACGGTAGCAAAATATATGT
TCAAGACCTAATCTGGGAAGAAAGAGAGACGATAGCAAAGGCAATTCTTG
AAGGAAAGGCGTATGTTTATATCTGCGGTGATGCAAAAGCCATGAGTCGT
GCAGTCGAAGACACCCTGGCCAGGATTCTGGGAGAAGCAAAAGGTGGCAA
TGCAGAAGTGGAAGGTGCAGCAGAGATGAAGATCTTGAAGGAACGTAGTA
GACTACTGCTTGACGTATGGTCT

Seq. ID No: 187

>T5H-CPR_7TCAAGCTCTTCCAGCAGCTCACTGTTTTCTACTACTGACA
TGGTACTGTTCTCTTTAATTGTCGGAGTCCTGACCTATTGGTTCATTTTC
AGAAAGAAGAAGGAGGAGATTCCCGAGTTCAGTAAAATACAGACTACCGC
CCCACCTGTCAAGGAGAGCAGCTTTGTGGAGAAAATGAAGAAAACCGGCA
GGAACATTATAGTCTTTTACGGCTCTCAGACCGGCACGGCGGAGGAGTTC
GCAAACAGACTGTCCAAAGACGCCCATCGTTATGGTATGCGTGGCATGAG
TGCGGACCCAGAAGAATACGACCTTGCGGATTTATCATCCTTGCCGGAAA
TTGATAAGTCACTTGTGGTCTTTTGCATGGCAACATACGGAGAGGGTGAC
CCAACGGACAACGCGCAAGACTTTTATGACTGGCTTCAAGAGACGGATGT
AGACTTAACTGGCGTTAAATTTGCCGTTTTTGGACTAGGTAATAAGACTT
ACGAACATTTCAACGCAATGGGCAAGTATGTTGATCAGCGTCTTGAGCAG
CTGGGGGCCCAACGTATTTTCGAATTGGGCTTGGGAGACGATGATGGCAA
CTTAGAAGAGGACTTTATTACTTGGCGTGAGCAGTTTTGGCCTGCAGTGT
GCGAGTTCTTCGGGGTGGAAGCTACCGGCGAGGAATCATCTATCCGTCAG
TATGAATTAGTGGTCCACGAGGACATGGATGTTGCTAAAGTGTATACCGG
CGAAATGGGCAGGCTAAAATCTTACGAAAATCAGAAGCCACCCTTCGACG
CCAAGAATCCATTTCTAGCAGCGGTCACTGCCAATAGAAAACTTAATCAG
GGGACTGAGAGACATCTAATGCATTTAGAACTAGACATCAGTGACTCAAA
AATAAGATATGAATCCGGCGACCACGTTGCAGTGTACCCTGCCAACGATT
CCGCGCTAGTAAACCAGATAGGTGAGATCTTAGGTGCGGACCTAGACGTA
ATCATGAGTTTGAATAACCTAGATGAAGAGTCTAATAAAAAGCACCCCTT
CCCTTGTCCTACAACGTATAGGACGGCCCTTACATACTACCTAGACATCA
CAAACCCGCCAAGAACTAACGTGCTTTATGAGTTAGCTCAATACGCCTCA
GAACCTTCTGAGCAAGAGCACCTGCATAAAATGGCAAGTTCCTCCGGTGA
AGGTAAAGAGCTTTATTTATCATGGGTAGTAGAAGCACGTAGACACATAT
TAGCCATTCTGCAAGACTACCCATCTTTGCGTCCTCCCATCGACCATTTG
TGCGAACTTTTGCCCCGTCTGCAGGCACGTTATTATTCCATAGCCAGCAG
TTCAAAGGTCCATCCAAATTCCGTACACATATGCGCTGTGGCCGTGGAAT
ATGAGGCGAAATCCGGGCGTGTTAATAAGGGTGTCGCGACAAGTTGGCTA
CGTGCCAAGGAGCCCGCGGGTGAAAATGGGGGCAGGGCCCTAGTTCCTAT
GTTTGTAAGGAAGTCACAGTTTAGATTACCATTTAAAAGTACTACCCCAG
TAATCATGGTTGGCCCCGGGACCGGGATCGCGCCTTTCATGGGATTCATT
CAGGAAAGAGCTTGGCTACGTGAGCAAGGAAAGGAGGTTGGCGAGACGTT
ATTGTACTATGGATGTAGGAGGAGTGACGAAGACTACCTATACCGTGAAG
AGCTGGCGAGGTTTCACAAAGATGGTGCATTGACTCAGCTTAATGTGGCA
TTTTCCCGTGAGCAGGCCCACAAGGTCTACGTCCAACATCTGCTTAAACG
TGATCGTGAGCATTTATGGAAACTGATTCACGAAGGCGGTGCTCATATCT
ATGTTTGTGGTGATGCTAGGAATATGGCCAAAGATGTTCAAAATACATTT
TATGATATAGTGGCTGAATTTGGGCCGATGGAGCATACTCAAGCCGTTGA
TTACGTGAAAAAGTTGATGACAAAGGGCAGGTACAGCCTGGATGTATGGT
CA

Seq. ID No: 188

>T5H-CPR_8TCATCTAGCTCATCATCTGGGGGCTCTCCCATGTCCGATT
CCGTTGTCGTAATCATCACGACGAGCTTCGCAGTCATAATCGGCTTACTG
GTGTTTCTTTGGAAACGTTCTTCAGACAGGAGTAAGGAGGTTACTCCGCT
TGTTGTACCAAAAAGTCTTTCCGTGAAAGACGAAGAGGACGAAGCTGAAA
CACTTGCGGGCAAAACAAAAGTCACAATTTTCTATGGTACTCAGACAGGC
ACGGCCGAAGGTTTTGCTAAGGCATTGGCGGAAGAGATAAAGGCTAGGTA
TGAGAAAGCAGCAGTCAAGGTTGTGGACTTAGATGATTACGCTATGGATG
ATGACCAGTACGAAGAGAAATTAAAGAAGGAGACTCTAACGTTTTTCATG
GTAGCAACTTATGGAGACGGTGAGCCAACAGACAATGCTGCAAGGTTTTA
CAAATGGTTCACAGAAGAGCACGAGCGTGGGGTGTGGCTACAGCAGTTGA
CTTATGGGATCTTCGGGTTAGGAAATAGACAATACGAACATTTTAATAAG
ATAGCCAAAGTACTAGACGAGCAATTGAATGAGCAAGGCGCCAAGCGTTT
AATTCCAGTAGGACTGGGAGACGATGATCAGTGTATTGAGGACGACTTCA
CAGCGTGGAGGGAACTTCTATGGCCCGAGCTAGACAATTTACTACGTGAC
GAGGATGACGTCAACGGGGCAAGCACCCCGTACACAGCGGCAATACCGGA
ATATCGTGTCGTAATCCATGACGCCAGTGCTACGAGCTGCGAAGATAAAA
GTGTGCTAGAGAATGGGAACACGTCCATCGACATCCACCACCCTTGCCGT
GTAAACGTAGCCGTTCAAAAGGAACTGCACAAGCCTGAAAGCGACAGGAG
CTGTATACATTTGGAATTTGACATTAGCGGTACAGGTATAATTTACGAGA
CTGGTGATCACGTGGGCGTGTACGCGGAGAATTTTGAGGAGAACGTTGAA
GAAGCAGGGAAACTTCTTGGCCAGCCTTTGGACCTATTGTTTAGTATTCA
CGCGGACAACGAGGATGGCGCCCCTTTAGGATCAAGTCTTGCACCACCTT
TCCCCGGCCCCTGTACACTACGTACTGCACTAAGCCATTACGCAGATCTG
TTGAACCCACCCCGTAAGGCGGCACTAATAGCTTTAGCGGCCCACGCCTC
AGAACCCTCTGAGGCAGAGAGACTTAAATACCTGAGTTCACCCGAAGGGA
AGGACGAGTATAGTCAATGGATCGTTGGCAGCCAAAGGTCACTGCTGGAA
GTGATGGCCGAATTTCCCTCTGCGCGTCCTCCTCTGGGCGTTTTCTTTGC
TGCTATCGCGCCCAGGTTACAACCTAGGTACTACTCTATTTCTAGTTCTC
CTAGGTTTGCACTGTCCAGAGTACATGTGACCTGTGCGCTAGTCTACGGT
CCAACTCCAACGGGCAGAATCCACAAGGGGGTATGTAGCACATGGATGAA
GAATGCGGTGCCGCTAGAGAAGTCACACGACAGTAGTTGGGCGCCCGTGT
TTATTCGTACCTCTAACTTTAAACTTCCAACAGACCCTTCAATACCTATC
ATTATGGTAGGTCCTGGGACCGGACTTGCGCCCTTTAGAGGTTTCCTGCA
GGAGAGGATGGCGCTTAAAGAGGACGGTGCACAATTGGGGCCAGCTTTGC
TGTTCTTCGGTTGCCGTAATAGGAGAATGGACTTTATCTACGAGGACGAG
CTTAACTATTTTGTGGAACAGGGGGTAATTTCCGAATTAATCGTAGCCTT
TTCTAGGGAAGGGCCGCAAAAAGAGTACGTCCAGCATAAGATGATGGACA
AAGCAGCCCAAATCTGGTCATTGATTTCTGAGCGTGGGTACATATATGTG
TGTGGCGATGCAAAGGGGATGGCGAGAGATGTTCATAGGACCCTACATAC
GATCGTCCAAGAACAAGGTAATTTGGACAGTTCCAAAACGGAGAGTATGG
TCAAGAAATTGCAAATGGACGGTAGATACCTTAGAGACGTATGG

Seq. ID No: 189

>T5H-CPR_9TCTTCCTCAAGTAGCTCCACTTCTTTCCACAAGTTAAAAA
GAATTTTACACAAACACTTGCAGCGTAGTCATTCCATCGGAGCAGAATGT
AAACCCCAAAGGTCCAATCACGAGGATCTATTAGCGGTGATGAACAGGAG
TTCAATTAAGGTTTCTATATTTTATGGTAGCCAGACAGGTACGGCGAAGA
AGTTTGCAATCAACCTTGGGCATCACCTGCATAACTGTGGTGTACGTAAC
TTGGTTATGGACCTGAGACAGACGAATATGGAAATCTTAGTGAACTTATC
TATGCTTGATAATTGCGTAGCATTATTTGTTGTAGCCACTTACGGAGAAG
GAGAACCGACGGATAGTGCTAGGCAATTTATGGACAACTTAAAGAACTCC
TATCAAAAGTTGGATAATCTTCGTTTTGCAGTGTTTGGATTGGGAAATAG
TATGTACACGTATTTCAATGCAGTGGGAAAGTCTATCGACCGTTTGTTGA
TTCAACACGGAGGAAAGCGTTTGCAAACGTTAACACTGGGCGATGAAGTA
AACGAGTTGGAGTCTACATTCCTAAACTGGCGTTCCCACCTTACCAGTTT
ATTAATCGATTTCTTTGACTTGAACGATCACGATAGAAACTACTTAAATA
AACAGTATAAAAGGATGTACTCACTGAAGAGATTTAACTGGAATGTGCCC
CTTGTTTCCCACTTCGTAAACATGTTTATCAACAAGGCACACGTTAAGGA
GACCCTTCCTTACGAAAATGACAATTACTTCTACGCTTCTGTCGCAGTTA
ATCAAGAGCTTTATCACAAATCCTCGCGATCATGTCGTCATATCGAGCTA
GATGTCTCCGCCTCTCAGCTTAGGTATAAGACTGGGGACCATATTGCTAT
TTTTGCGTCAAACCCATTGGACCTTGTAGAGAAAATAGGAGATCTGCTGA
ACATCGACCTTAACGAAATGATCTCTTTGGACGCTGTCGATCCAGACTCA
CTAACAAAGCACCCCTTTCCTTGCCCGTGCACGTATAGACACGCATTTAT
GCATTTTGTCGATATAACGGGTCCTCCGGGTAAGTCCCTGTTAAGTGCAT
GTCTGGATAGCGTCACGAACCCCGAGGAGTCCCAGTTTGTGCAATTGCTG
ATCTCTGACAGTGAGGACGGCAAGAAACTGTACAGTAAATGGATATTGGA
AGACCATAGGGGATTAGTCGATGTACTTCAGGATCTAAAGAGCTTCAGGC
CGCCGGCAGACCTTCTATTGGAATTATTAAATCCGCTGAAACCGAGACTA
TATTCTATTAGTTCCAGTAGTCTTGTGCATACGAATCGCATTCATATTAC
AGCCTCTATTGTCAAGTATAAGACGAACTCTGGTAGAATCTTTAAAGGGC
TGGCCACGAACTGGTTAAAATCCTTGCAGTCAACAAACACGGAGCGTCAC
CTAAAGATCCCAGTTGCCATACATACGTCCAACTTCAATTTACCGCGTTC
CAGGACCATTCCAGTGATAATGATAGCAAGCGGTACCGGGTTGGCTCCAT
TTAGAGCCTTTATCCAGGAGAGACTCAAGGTGGCGCACGACAAAGTCGGA
AAGACCGGACAGATGGTATTATTCTTTGGGTGCAGGCACGAGAATAAAGA
TTTCATTTATTCAGACGAGCTTAAACAAGCGTGCTCTACCGGCCTACTTG
AAATGTTCACGGCATTCTCCAGGGATTGTCTGGATGGCAATAAGGTCTAT
GTACAGCATAAAGTTTTGGAGATGGGCAATATGGTATGGAAGTTATTAGA
CGAATGTTACGCCTACATATATGTGTGCGGTGACGCCGCAGGAATGGTTC
GTGATGTACATCTTTGTTTAATCGAACTTGTTGTTCAGAGGAGTAACCTT
ACGCGTGAAGCGGCCACGTCCTACGTTTTAAACTTGCGTAAACAAGGTAG
GTATCGTACCGATGTTTGGAAA

Seq. ID No: 190

>T5H-CPR_10TCAAGCTCCTCTTCCTCCGGGGGCAAAATATTTGATAAA
TTGAACTCTTCTCTCGATTCTGGGGACAGCACTAGCCCTGCATCACTGAC
GGCTTTATTAATGGAAAATAAGGATCTTATGATGATTCTGACAACTAGCG
TTGCCGTCCTAATAGGGTGCGCGGTTGTCCTGATGTGGAGAAGATCCAGC
ACATCCGCGCGTAAGGTGGTAGAACTTCCCAAACTTGTAGTTCCCAAGTC
TGTTGTAGAGCCTGAAGAAATCGACGATGGCAAGAAAAAGATAGCGATCT
TTTTCGGGACTCAGACCGGCACGGCTGAGGGTTTTGCTAAGGCATTAGCA
GAGGAAGCCAAGGCAAGATATGAAAAAGCAATATTTAAAGTCATTGACAT
GGATGACTACGCGGCTGATGACGAAGAATATGAAGAGAAGCTCAAAAAGG
AGAAATTAGCATTTTTCTTTCTGGCGACCTATGGAGACGGCGAGCCCACA
GATAATGCGGCCAGGTTCTACAAGTGGTTCGAGGAGGGCAAGGAGAGAGG
AGACTGCTTCAAAAACCTACAATACGGTGTCTTTGGCCTAGGCAACCGTC
AATATGAGCATTTTAATAAGATCGCGAAGGTGGTCGATGAACTGCTTGCT
GAACAAGGCGGGCAGCGTCTAGTCCCAGTAGGATTAGGGGATGATGATCA
GTGTATAGAGGACGATTTTGCTGCTTGGCGTGAGTTGGTCTGGCCCGAGC
TAGATAAGTTGCTGCTAGACGGTGATGATGCCACAGCAACGACCCCCTAT
ACTGCTGCAGTGCTAGAGTACAGGGTGGTCACGTACGATAAGAGCAACTT
CGATAACGACTTGACTAACACGAACGGCCATGCGAACGGCCACGTCATTG
TTGATGCCCAACACCCAGTTCGTGCCAATGTTGCGGTTCGTAAGGAGCTA
CACACCCCCGCGTCTGATAGGTCCTGCACCCACTTGGAGTTTGACATAAG
TTGTACAGGACTTACTTATGAGACTGGAGATCATGTTGGCGTGTACTGCG
AAAATTTTGTGGAAACGGTTGAAGAGGCGGAAAGACTTCTGAACATATCA
CCTGACACATTCTTCTCCATACACACCGACAAGGAGGATGGAACGCCTTT
GGGAGGGAGTAGTTTACCTTCTCCATTTCCACCCTGTACACTAAGAACGG
CGTTGACTAGATATGCAGACGTCTTGAGTTCACCAAAGAAGTCCTCCCTG
CTTGCCCTGGCGGCCTGTTCATCAGATCCCAATGAGGCAGATAGACTGAG
ATACTTGGCCTCTCCCGCGGGAAAAGAGGAGTATGCGCAATGGATAGTTG
CCAGTCAGAGAAGTTTGTTGGAGGTTATGGCGGAATTTCCGTCAGCTAAA
CCGTCCATAGGGGTGTTTTTCGCAAGCGTTGCACCTAGATTACAGCCGCG
TTTCTACTCAATCTCTAGTTCTCCGAGAATGGCTGCATCCCGTATACACG
TAACTTGCGCTTTAGTATACGATAAGATGCCGACGGGACGTATCCATAAA
GGGGTTTGCAGCACCTGGATGAAAAACGCCATACCGTTGGAAGAAAGTCT
AAGTTGTAGCACCGCCCCGATCTTTGTTAGACAGTCAAATTTTAAACTTC
CAGCCGACAACAAGGTTCCTATCATTATGATTGGTCCTGGCACGGGATTG
GCGCCGTTCAGAGGCTTTTTGCAGGAGAGGATGGCTCTGAAGGAAGAAGG
TGCGGACCTGGGGCCCGCCGTGTTGTTCTTTGGGTGCCGTAACAGACAAA
TGGATTACATATATCAGGATGAATTAGATAACTTCCTTGAAGCCGGGGCG
TTAAGTAATCTAGTCGTCGCTTTTTCAAGAGAAGGACCCAACAAGGAATA
CGTCCAACACAAGATGACACAAAAGGCAGATGATATTTGGAATATGATTT
CTCAAGGGGGTTACGTTTATGTCTGCGGTGACGCAAAAGGCATGGCCAGA
GACGTACACAGAACACTTCATACTATCGCGCAGGATCAGGGGAGCCTAGA
CTCCTCTAAGGCAGAATCATTCGTCAAGAATCTGCAGACAACCGGTAGGT
ACCTAAGGGACGTGTGG

Seq. ID No: 191

>T5H-CPR_11AGTTCATCTTCTTCCAGCGGGGGAGACGGTGCCGAAGGC
AGGGCTCTGGTAGCCACTCTAGCTGCGGCGGTGCTTGGAGCGGCTTTATT
TGTCTTATGGCGTAGGGCCGCTGCAGGCAAGAAGAGGAAAAGGGAGGCCG
CGGCTGCTGCAGTGGCTGAGGCGACAGAGGTCAAAGCAAGAGCGGCCAAA
GGTGGCGAAGATGAGAAAGCCGCCGATGACGGACGTAAGAAGGTAACAGT
TTTCTTCGGTACGCAAACTGGAACAGCCGAAGGTTTTGCGAAGGCCCTGG
CAGAGGAGGCTAAAGCGCGTTACGACAAGGCAATATTTAAGGTTGTAGAC
TTAGATGATTACGCTGCCGAAGATGAGGAATACGAAGAGAAATTGAAGAA
GGAAAAGCTGGCTCTGTTCTTTGTCGCTACGTACGGGGATGGGGAACCTA
CTGACAATGCCGCAAGATTTTATAAATGGTTCACGGAAGGTAATGAAAGG
GGTGTCTGGCTGAACGATTTTGAGTATGCGGTGTTCGGTCTAGGCAACCG
TCAATACGAGCATTTCAACAAAGTAGCGAAAGTTGTTGATGAAATTCTTA
CAGAACAAGGGGGCAAGAGACTGGTACCTGTCGGACTTGGTGACGATGAT
CAATGCATCGAGGATGATTTTAATGCATGGAAAGAGGCGCTGTGGCCCGA
GCTGGATAGACTTTTGCGTGATGAGAACGATGCCAGCACGGGGACAACTT
ACACGGCAGCAATCCCGGAGTACCGTGTCGAGTTTATAAAGCCTGAAGAG
GCGGCTCACCTGGAGAGAAACTTCAGCCTTGCTAACGGTCATGCCGTGCA
TGATGCTCAGCATCCGTGCCAGGCTAACGTGGCGGTTAGACGTGAGTTAC
ATACGCCGGCGTCTGACAGGTCATGCACTCATTTGGAGTTCGACATTGCA
GGCACCGGACTTACCTACGAAACGGGGGACCACGTTGGAGTGTACACAGA
GAATTGCCCGGAGGTGGTTGAGGAAGCCGAGAGGTTGTTGGGTTACTCCC
CGGACACTTTCTTCACAATTCACGCGGATAAAGAGGATGGTACTCCCTTG
TCCGGCTCATCCCTAGCGCCACCATTTCCGTCTCCCATCACGGTAAGGAA
CGCCTTAGCCAGATATGCCGATTTACTGAACTCTCCCAAAAAGACCAGCC
TGGTAGCCCTAGCGACATACGCCAGTGATCCCGCTGAGGCGGACAGGTTA
AGGTTCCTGGCTTCTGCAGCGGGGAAAGATGAATATGCTCAATGGGTCGT
GGCGTCACAAAGAAGTTTACTAGAGGTCATGGCGGAATTTCCATCTGCCA
AACCGCCACTTGGCGTGTTTTTCGCCGCCGTGGCGCCAAGGTTACAACCC
AGATATTATTCAATTTCCTCATCCCCATCGATGGCTGCCACGAGGATTCA
CGTAACTTGTGCCTTGGTTCATGAAACCACTCCAGCCGGGCGTGTACACA
AGGGAGTTTGCTCAACATGGATCAAAAATGCAGTCCCCTCTGAGGAGAGC
AAAGATTGTAGCTGGGCGCCGATTTTCGTAAGGCAAAGCAATTTCAAATT
ACCGGCCGATCCTTCAGTTCCCATAATAATGATCGGGCCGGGAACAGGCC
TTGCACCGTTTCGTGGCTTTTTGCAGGAACGTCTGGCCCAAAAGGAGTCT
GGAGCAGAATTGGGCCCAAGCGTCTTTTTCTTTGGTTGTAGAAATTCAAA
AATGGATTTCATCTATGAAGATGAACTTAACAATTTCTTGGAACAGGGTG
CCCTATCTGAACTTGTTTTAGCGTTCTCCCGTCAAGGGCCAACTAAGGAG
TATGTTCAGCACAAAATGGCTCAGAAGGCGTCTGAAATATGGGACATGAT
TAGTCAGGGGGCGTACATTTACGTTTGCGGGGATGCGAAGGGCATGGCCA
GAGACGTGCACAGAGTCCTACATACAATCGTCCAAGAACAAGGATCACTG
GACAGTTCCAAGGCCGAGAGCTTCGTGAAGAACCTGCAGATGGAAGGACG
TTATCTACGTGACGTGTGG

Seq. ID No: 192

>T5H-CPR_12TCCTCATCTTCTTCCTCAGTAAGAGAATCTAGTTTCATA
GAGAAAATGAAAAAGACCGGTAAGAATATCGTAGTGTTCTACGGATCACA
AACTGGTACCGGTGAAGAGTTTGCGAATAGACTTGCAAAAGACGCTCACC
GTTATGGAATGAGAGGGATGGCTGCTGATCCCGAAGAGTTCGAAATGACG
GACTTGTCCAGATTAACAGAGATCGAAAATGCACTAGCAGTTTTCTGCAT
GGCTACCTACGGTGAAGGTGACCCCACTGATAATGCGCAAGACTTCTATG
ATTGGCTGCAGGAGACAGATATCGATCTGGCCGGGCTAAAGTATGCCGTG
TTTGGCCTGGGAAACAAAACGTACGAACATTTTAACGCTATGGGCAAGTA
TGTGGATAAGAGATTAGAGGAGTTAGGGGCAGAAAGAATTTTTGAGTTAG
GGATGGGGGACGATGACGGTAACTTGGAAGAAGACTTTATCACGTGGAGA
GAACAGTTCTGGCCGGCGGTTTGTGAACACTTTGGCGTCGAAGCGACAGG
AGAAGACAGTAGCATTAGACAATACGAATTGGTCGTACATACGGATGAAA
ATATGAACAAGGTATATACAGGCGAAATGGGGAGACTAAAGAGCTACGAG
ACCCAGAAACCTCCTTTTGACGCCAAGAACCCCTTCCTTGCGAACGCCAC
TGTCAACAGAAAGCTGAATGAAGGGGGAGATAGACATTTTATGCATCTTG
AACTAGATATTACGGGCTCAAAGATTCGTTATGAGAGTGGCGATCACGTC
GCAGTCTATCCTGCTAACGACGCGGCACTGGTTAATAAATTAGGAGAAAT
TCTTGGAGCCGACTTAGAGACAGTTATTTCTCTGAACAATCTTGATGAAG
AGTCTAATAAGAAACATCCCTTCCCTTGTCCGACTACCTACAGGACGGCA
CTAACGTACTATCTGGATATCACGAATCCCCCGCGTACAAATGTTTTATA
CGAGTTGGCACAATACGCCACCGATTCAAAGGAGCAAGAGAACCTACGTA
AAATGGCGTCATCTGCGCAAGATGGTAAAGCACTGTACCTGTCTTGGGTG
GTAGAATCCAGGCGTAACATATTAGCTATATTAGAAGACATACCATCACT
GAGACCGCCTCTGGACCACTTGTGTGAACTTTTACCCAGGTTACAGGCGA
GATATTACTCTATAGCAAGCTCCAGCAAAGTCCACCCTAATAGTATTCAC
GTTTGCGCAGTCCTGGTGGAATATGAAACCAAAACTGGCCGTGAAAACAA
AGGCGTCGCCACAAATTGGCTGAAGAATAAGCAACCTAGCGATAACGGTC
ATAAATCCTCAGTACCAATGTTTGTAAGAAAGAGTCAATTTAGGTTACCA
TTTAAGCCATCCACACCCGTTATAATGATCGGTCCGGGTACCGGCATTGC
GCCCTTTATGGGATTTATACAAGAACGTGAATGGTTAAAGCAGCAAGGTA
AAGACGTCGGCGAGACGGTACTATACTATGGTTGCAGGCATGAACATGAG
GATTTCTTGTACATAAACGAACTGAAAAGGTATCACAAAGAGGGAGTTCT
AACGCAGTTGAACGTCGCGTTCTCTAGGGATCAAGCACACAAAGTGTACG
TACAACACTTGCTAAAGAACAATAAGGAAATGGTTTGGAAGTTAATCCAC
GAGGATAACGCTCATATTTATGTCTGCGGTGACGCCAGGAACATGGCCAG
AGACGTACAAAATATCTTTTATGACATCGTTGAGGAATACGGCAAGCTAG
ATCACGCCCAAGCCGTAGACTACATAAAGAAATTAATGACTAAGGGGAGG
TATTCACAGGATGTGTGGTCT

Seq. ID No: 193

>T5H_1ATGCTACCGATCGTGGATCACCTACTTGACGTTTTAAACTTGGA
ACGTACACCCTTTCGTACATATGCTGTGACGGCATTACTGCTTCTTTTCG
TAGGCATCATTGCCAGAGCCCTACTTAAAATGATGCTATTTATTCAGGAG
TACAGTGCTAATTCAAAGCGTTTGAGGTGTTTCCCCGAGCCCCCGAATAG
GTCCTGGATCTTGGGGCACCTGGGTCTTTTTGCCCCGAACGAGGAGGGAA
TGACAGAATTTAGTAAACAGGTGAGCAAATTTACCTACTACATGAAGACA
TGGATGGGACCAGTAATACCGCTGATTAGTCTAATACATCCCGATACTAT
TAAACCAGTAGTTGCGGCACCAGCTTCTATCGCGCCTAAAGACGCACTTT
TCTACGGATTTTTAGAACCGTGGCTGGGCGACGGTCTACTTCTAAGCAGG
GGCGAGAAGTGGGTTCGTCATAGACGTCTACTGACGCCCGCTTTCCATTT
CGACATCCTGAAGCATTATGTCAAAATATTTAACCAGTCCACAGATATTA
TGCATGCAAAGTGGAGACGTTTGTGTACAAAGGGGCCTGTCTTCCTTGAT
ATGTTCGAGCACATAAGTCTAATGACCCTGGACTCCTTATTAAAATGCAC
GTTCTCCTATGATTCTGACTGTCAGGAAAAGCCTTCAGATTATATCGCGG
CGATATACGACTTGTCAGAGCTTATAGTGGAAAGAGAGCAATGCCCGCCT
CATCACTTCGATTTCATCTATAGATTTTCTTCTAATGGGAGGAAGTTCCA
GAGGGCCTGTCGTATCGTCCACGAGTTCACTGCAAATGTCGTCCAGCAGA
GAAAGAAAGCGTTGCAAGAGAAAGGAGCGGAAAACTGGATAAGATCCAAA
AAGGGCAAAACTCAAGACTTTATTGACATCCTTCTTTTAAGTAAGGATGA
AGACGGTAATACTCTGTCTGACCAGGAGATGCGTGATGAAGTCGATACGT
TCATGTTCGAAGGGCACGATACTACCGCTTCCGGTCTGTCATGGATACTT
TATAACTTGGCATCACATCCCGAATATCAGGAGAAATGTAGGGAAGAGGT
GACACAACTGCTGAAAGGTGAGTCTACACACCTTGAGTGGGATGACTTAT
CCCTTCTTCCGTTCACCACTATGTGCATAAAGGAAAGTCTAAGGCTGCAC
CCGCCGGTAACGGCAGTGAGTAGAAGATGCACTGAGGACATCGCAATGCC
AGACGGGAAGGTAATTCCGAAAGGCAATATCTCTCTTATATCTATTTACG
GTACTCACCACAATCCAGCGGTTTGGCCTAACCCGGAAGTCTACGATCCT
TACCGTTTCGATCCGAGTAGCACAGATGAAAGATCCTCCCACGCCTTCGT
TCCTTTCAGCGCGGGCCCTAGAAACTGTATCGGGCAAAATTTCGCTATGG
CAGAGATGAAAGTCGTCCTAGCCCTAACTCTTCTTAACTTTAAGGTTGCG
TTAGACCCAAACAGGGTGGTCCGTCGTAAGCCGGAGTTAGTTCTTAGAGC
GGAAGGTGGACTATGGTTGCAAGTCGAAGCCCTTAAAAGCAAGTCA

Seq. ID No: 194

>T5H_2ATGGAGCTGTTGGGCTTGGTCTCTTGGCTTCTGCTTCTTCTACT
GACTTTGGTGGTTATCTGCTTTCTACTGTACTGTGGCTACATCCATTACC
AGCACATGAAATATGACCACATACCCGGACCGCCCAGAGAGTCATTCTTG
TTTGGACACGGTTCAGCTATCTGGAAAGTGATGAGAAAGAACCAATTAGT
TTATGATTTATTTCTAAATTGGGTCGAAACATATGGGCCTGTTATACGTA
TAAATGCACTGCATAAAGTAACCATCGTTAGCGTCTCACCAGAGAGTGTC
AAGGAAGTCTTAATGTCTCCAAAATACCGTAAGGACTGGTTCTACGACCA
TTTGCACAGTCTATTCGGAGTCCGTCTAATGGGAAATGGTTTAGTGACAG
ATAGGGACAATGATCACTGGTATAAGCAGAGAAGAATCATGGACCCAGCA
TTCAGCAGAACGTACTTGATTGGGCTACTAGGGCCGTTTAACGAGAAGGC
TGAAGAACTGATGGAGCGTTTAGCAGAGGAAGCTGATGGGAGGAGTCATG
TAGTTATGCACGCCATGATGAGTAGAGTGACTTTGGACGTAATTAGTAAA
GTGGCCTTTGGAATGGAGATGAACTCCTTGAAGGATGATGGGACGCCACT
TCCCAGGGCGATCAGTCTTGTAATGAGGGCTCTTGTGGAGATGAGAAATC
CATTCATCAGGTACAGCAGAGAAAAACAAGCATTCATACGTGATGTTCAA
GAGTCTGCCCGTTTATTGCGTAAAACTGGTCGTGAATGCATAGAGCGTCG
TCAAAAGGCGATACAGGACGGTGAGGAGATACCTGTTGACATATTGACCC
AAATCTTGAAAGGCGCCGCGTTGGAAGGGGACTGTGACATGGAAGATCTG
CTGGATAATTTTGTGACGTTCTTTATTGCCGGCCAGGAAACAACCGCGAA
CCAACTTGCGTTTACTATAATGGAGTTAGCGCGTAACCCCGAGATTCTGG
AAAAGGCTCAGGCAGAAGTAGACGAAGTCATTGGAGTCAAGAGGGATATA
GAATACGACGACCTAGGCAAGTTGCAATACTTATCCCAGGTCCTGAAAGA
GTCTCTTAGGCTGTATCCGACTGCTCCTGGGACAAGTAGGGCAATAGAGG
AAGAGACGATAATAGAGGGTTTTCGTATTCCCCCTAAAGTCCCGTTAATG
TTCAACAGTTACATCATGGGGAGGATGCAACAGTTTTATCCCGATCCCCT
TACATTCAACCCGGATAGATTCCATCCAGATGCTCCCAAACCGTACTATA
GCTACTTCCCGTTTTCCTTGGGTCCGAGAAGTTGTATCGGACAGGTCTTC
GCACAAATGGAAGCTAAAGTCATCATGGCCAAACTATTGCAAAGATTTCA
ATTCGAGCTAGTAGAGGGGCAGAGCTTCGGTATCATGGATACCGCCAGCC
TTCGTCCGGAGGGCGGGGTTATTTGTCGTCTTACGATACGTACGAATCCG
GGAAAGGCCAAGAAAGACGAT

Seq. ID No: 195

>T5H_3ATGAGCAGGCCTCAAGTCCCAAAGGGGCTAAAGAACCCTCCAGG
CCCGTGGGGCTGGCCCTTGATCGGCCATATGCTAACCTTAGGGAAAAATC
CACACTTAGCTCTTAGTAGAATGTCACAACAATATGGCGACGTTTTACAG
ATCCGTATTGGGAGCACACCGGTAGTTGTACTTAGTGGCCTGGATACCAT
AAGGCAGGCGTTGGTTAGACAAGGAGACGACTTTAAGGGGAGACCTGATC
TGTACACGTTTACTTTAATTTCCAATGGACAATCAATGAGTTTTAGCCCC
GATTCCGGTCCTGTATGGGCCGCCAGGAGACGTCTTGCTCAAAATGGACT
AAAGTCTTTTTCAATTGCTAGTGATCCCGCCTCTTCTACGTCTTGTTATC
TGGAGGAGCATGTTTCAAAAGAAGCCGAGGTTTTGATCTCAACTCTTCAA
GAATTGATGGCCGGACCAGGACATTTCAACCCGTATAGGTACGTGGTAGT
ATCTGTAACGAACGTCATTTGTGCGATATGCTTCGGGAGGAGGTACGATC
ATAACCACCAAGAACTACTATCTTTAGTTAATTTGAACAATAATTTTGGA
GAAGTTGTCGGGTCTGGGAACCCGGCGGACTTTATACCTATTCTAAGATA
TCTACCGAATCCCTCTCTTAATGCCTTCAAAGACTTGAACGAGAAATTCT
ATTCATTTATGCAGAAGATGGTGAAAGAGCACTATAAGACGTTCGAGAAG
GGGCACATTAGAGACATTACAGATTCTCTAATAGAGCACTGCCAAGAGAA
GCAGCTGGATGAGAATGCAAACGTTCAACTATCTGACGAGAAGATTATTA
ATATCGTGTTAGACCTATTTGGCGCTGGCTTTGATACGGTCACCACAGCT
ATTTCTTGGTCCCTGATGTATTTAGTGATGAACCCTAGAGTTCAGAGGAA
GATACAAGAAGAGCTGGATACCGTAATCGGGAGATCCAGACGTCCGCGTC
TGAGTGACAGAAGTCATTTACCCTACATGGAGGCCTTTATCTTAGAGACT
TTTCGTCACAGCTCCTTAGTGCCTTTCACTATCCCACACAGTACGACGAG
GGATACATCTTTGAAAGGTTTCTACATCCCAAAAGGGAGATGCGTATTTG
TCAATCAGTGGCAAATAAACCACGACCAGAAACTATGGGTTAATCCAAGC
GAATTTCTGCCTGAGCGTTTCCTTACTCCCGATGGCGCAATAGATAAAGT
GTTATCCGAGAAGGTCATCATCTTCGGTATGGGGAAAAGAAAGTGTATTG
GGGAAACAGTAGCGAGGTGGGAAGTATTCCTTTTTCTGGCCATTTTATTA
CAGCGTGTAGAATTTAGTGTACCACTGGGGGTTAAGGTAGATATGACTCC
CATATATGGGCTTACGATGAAACACGCGTGCTGTGAACACTTCCAAATGC
AGTTGCGTTCC

Seq. ID No: 196

>T5H_4ATGCCGACTCCTGGAGGCAGATTGGTTGCCTTCCTGCAGAGGAG
GGGCAAGCTGGCAGGTAGTCTTGCTGTGATACTACTGTTAATACTGAAGA
GATTGAGGGACGCTCCAAGAAAGGTGAGGTGGTTGAGAGGACCGCCACTA
CTTGGGGTCGTGCTAAAAGTTTTTCAGGGACTGCGTGAGCACGCCTTGCT
AGATATGTATGACAGGTGGCATCAAAGGCTTGGACCCACTTTTGCATATT
GTGCCCCCGGAAAAATGGTTGTAGCAACAATCGATCCCAAAAACATAGAG
CACGTTTTGAAGACGAAATTTGATAATTACGTCAAGGGGCACGTGTTTGC
CGAACCGTTTACGGATCTACTGGGCGATGGTATATTTAACGCAGACGGGG
AGATGTGGCATAGACAAAGGAAAACAGCCAGCAGGATGTTTACCAAGCGT
CAATTCGAGACACATATATGGAAAGCAATAGAAGCGAACACGGCCAAAGT
AGGTAGGATATTGGAACGTTCCGAGGGAACCCTTGATATGTTCAATCTTA
TGAACAGGTTTACCCTAGATACGATCGGGCGTATTGGATTTAGCAAAGAT
ATTGGTTCTTTAGAGGACCCGTCATCACCCTTCCTGAGATCCTTCGACAG
AGCACAACAAATTCTGATCTTACGCTTTTGGACTAATCCCGCTTGGAAAG
TTCTAAGGTGGTTAGGGGTGGGCTGGGAACGTGAATTGAAGGAACACCTA
GGTAGACTAGATGGGTATGCACGTGGCATAGTCAGGGAACTTAGGCAAAA
AGCGGAAGCTGGACAGGATGATTCCTTTGTAGGGTTGTTCATGAAGGAAG
AGCAAGCGGCTCCTGCAGCTAGATCCCCCGAGCTACAAGAGAAGTTTATG
AGGGACTTAGTACTGAATTTCTTGATAGCAGGCAGGGACACTACTGCACA
ATGTATTAGTTGGACTCTTTTCGAACTGACTCAGCACCCTGCCGTTGCGG
CGAAAGCAAGGCAGGAGGTTTTAGATGTATGCGGCGAGGGCCCCGTCACT
TTCGAGCATCTAAAATCCTTACAATACGTGAGAGCCATTTTAGACGAAGG
CCTGAGACTTCACCCGTCTGTTCCTTACGACGGCAAACTATGTCTAGGCA
AGGACACGTTGCCTGACGGCACTGTCGTGCCGGCCGGCTGCATAATTCAA
TACATCCCTTACGCTCAGGGCCGTTGTAAAGACATATGGGGTGAGGACGC
TTGTTCCTTCAGACCTGAAAGGTGGCTAGAGATGCCCCGTAGACCATCCT
CTTTCGCCTTTGCGGCCTTCAATGCTGGTCCCAGGGAGTGTCTTGGACGT
AGGCTTGCGGAAGCGGAGATGGCGGCTCTTGTTTCCACAGTCGTTCGTGA
TTTCGACATGCGTTTAGAGGTAGAGCCATCTAGTGTGCGTTATGATGCGC
AGTTAACATTAGGGATGTGTGGGTTGCCCGTTAGCGTGAGGCGTTGTCGT
AGGGCCTATGGTGTTGCTGAACCACTTGCCGGAGCG

Seq. ID No: 197

>T5H_5ATGCTACCGTTGAGACATAAGATGCTTACGGGAGAGGCCGAACC
CTGTCTAGTCTCAAAGACTGCAGAGACTGACGCTGAGTGGACCAGGGACG
CGTTTGGTATGGGGCAATACACAGCCGGACGTTGTGACCACTTATTATCC
TGGGTGGTCTTCTTACTTCTGGCCCCTGTACTATTGATAGTGTGGTTACC
TCTATCTTGTATCTGCTGTGCCTCTCCGGTATTATTAGTCCAGAGATTCG
CAGGATGGGTTCTTTCCGGGTGTTTGGCAAGGACATATTTGGGAGTCCTG
TTGATCAGATTGTGCGGGAAGTGCGACCTAATCCTTACAGGTATGCATTT
TATTAGGACGGGAAGTCAAAGGTTCTGGATGGATACACTTGACCCTCAGG
ATTGGGCGTATCACAACGAGACGTATGGGAGAAACATCATCCTATGGGCG
AATCTGCGTGTAGGATCATACAAGCAAGTTAGGGATATTGTTCTTAACCC
CGCGCGTAAAAGAACAAGGGCGCTGGACGGCTGGATATCAGGGTTCGCGA
GGCACTACCCCAATCTGCCCGTGTTTTTCAACACTGGCTCAAACATGCAT
ACTACGTTCAGGCAGATCTTTTTCGCCAACTTCACCAAAACGGATTTCGT
ACTTAGGGCGTTGGAAGACGAAGGAGCGGGCTTGGCGAAAATGGCGGCGC
CTATTTTGCAAAGGTGGCTTGCAGGGAGCTTTCGTGAAAGCAAGTCAGGT
GAGGGGAATTTATATATGGTGGAACCAGTGGCACCTCTGATTTTGTTTCT
GCTGTTCGAAGTAGAAGTGGAGTCCATTCCGCCCGAATTGTTGACCGCCT
TCTCAGACGTAGTAACTGTCGGTGCGTCATACTTTTTGCTGCCCCCACAC
TCACCGTATTGGCTACTGAGTGGAAAGGTGAAAGCTATTGCTCTGCTTAA
AGATTTCCTACTAGAACACTGTAACGCGGCGAGACCCGAGTCACTGAAGG
GCCGTGCTGTCGATTGGAGGAGCTTGGCGGCACAAATGCCCGCTTTTCTT
CCTAAGGACGAGTGTCGTTGTCCATGTAGTGGGACCCCGGCTGTAGATCC
CGTCGATGCGTATTTGGAAGTCATATCAGTAATGGTTTGCGTTGCGGGTG
TGACTGGCACTACTAATGGGTTCACATCAGTCATAAGAAAATTCGCCGAC
GTACCTGTAGGACCTACAAAGTCCCGTTGGCCTAGTGCGCCTGTACAATG
GCGTCCAGACGCTGATGATATGGTGAGACTTTACCGTAGGGACCCACTGG
GTTTTATCTTGGAGGCCCTTCGTCTTGGTACACCTGTTGCAGGGACCCAC
CAGGTCTTAGAGGAGGAATTAACTTGTCCTTTCCTGCACAAGGAGACAAC
GTTCCCAAAGGGGACGGTCGTATGCGCAAATCTAAATGCCTGCCACACTG
ATCCCGAGGAGTGGGGATCAGATGCTCTGGAATTTAGACCAGGAAGGGCT
GCAAGGAACCGTTACCTGATGTGGAACGGACCCTTCGGTGAGGCCGCGCC
GAGGCAGTGTCCCGGCGAACAGGTCGCGGCACATTGCATCAAAGTTTCTA
TCGATGCGTTCTTAGACATGCACAAGCCTCAA

Seq. ID No: 198

>T5H_6ATGGCTACGTCTATCCTATCCCTGAGCCTTATGGACTTGTTATA
TTGGGGTGCGTGTTTGTGCGTACTAAGTGTTTTGTACAAGATCTCAGCCC
TATACCTTAGACAAAAGAATTTTGAAAGGGTTTTCTCAGCATTTCCCGGA
CCGAAGAGGCACTGGTTGTATGGCAACGCTCACGAGTTCAAGCAAGACGG
AACAGACTTAGATATCCTGAACGGGTACGCAAAGCAGTTTGATTGCGCCT
TTCCATTGTGGCTTGGAAACTTTTTCGCCTCCCTAGCTATATACCACCCA
GACTATATAAAAGCTATCCTTAGTCGTCAAGACCCCAAGGATAACTTCGT
TTATCACTTCATCACCCCGTGGATCGGCAAAGGACTACTTGTCCTAAGTG
GCCAAAAATGGTATCAACATAGGAAACTTTTAACTCCCGGGTTTCACTAT
GACGTCTTAAAACCTTATGTTGGCGTTATGAGTGATTGTGTTAACGTTAT
GTTGGATAAGTGGGAGCGTCTAGTCCCCGACAAGAAGCCAGTGGAACTGT
TTCACTATATTTCCCTAATGACGCTAGATACTATAATGAAATGCGCCTTC
AGTTACCAAAGCAACTGTCAAAACGACTCCGAAAATGAGTACATTAAGGC
AGTTTATGAGTTGTCCTACTTGGTGGATCACCGTACTAGATGCCCTCCAT
ACCACAATGACTTTATTTTCTACTGGAGCCCGCACGGATTCAGACTGCGT
CGTGCTTTGAAGACGGCTCATCAACACACGGAGAAGGTAATAAAGTTGAG
AAAAGAATCTCTTAAACAGGAGACGGAGCTTGAGAAGATAAAACAAAAGA
GGAGGCTTGATTTCTTGGATATTCTGTTGTGTGCGAGAGACGAGAACGGA
CAAGGCCTTAGTGATGAAGATTTGCGTGCGGAGGTCGATACCTTTATGTT
CGAAGGGCATGACACTACTGCTTCTGGGGTCTCCTGGACGTTTTATTGCT
TGGCAAAGAACCCAGAACATCAGGAAAAATGTCGTGAGGAAATACGTCAG
GTCCTAGGCGACCGTAGAACCGTAGAGTGGGAGGACTTGTCTAAGTTACC
CTACACAACCATGTGCATCAAGGAGTCCATGCGTTTATATCCTCCGGTAC
CTGAGGTGGCAAGAGAATTAAAAGAGCCCATTACTTTCTGTGATGGCAGG
AGCGTGCCAAAAGGTAGTATCGTTTTCTTATGTATCTATGCTATCAATAG
ATGCCCTGGCATCTGGGAAGATCCTGAGGTTTTCGATCCGTTACGTTTTA
GCCCCGAGAACTCTTCAACGAGACATTCCCATGCTTTTCTACCTTTTTCT
GCGGGAGGCCGTAACTGCATAGGTCAGAATTTCGCGATGAACGAGATGAA
GATAGCCACTGCACTGACCCTACAGAGGTTCGAATTGCAATTGGAGACCA
AAAGAGAGCCAGTCAAACGTGCTCAGTTAGTGTTAAGAAGCATGAATGGA
ATCTATATCAACCTTAAAAAGATTCATAGCGACAAGACTAAGATTATC

Seq. ID No: 199

>T5H_7ATGGGTTTATGGACCTTTATGACAGGTGCCTTAATATTATTACT
AATTCTAGTGGTACTATGTTTTCTGCTGTACTGCGGTTACATATATTACA
TGCATATGAAATATGACCATATCCCCGGTCCTCCGAGAGATTCCTTCTTC
TTTGGTCACAGTCCGACCATTATGAAATTAATGAGAAATAACGTGATTAT
GTACGATACGTTTTTAGAATGGGTTAAGACATACGGACCGGTCGTTCGTG
TAAATCTATCATGTAGTACCATTGTTTTTGTCATCAGTCAAGAAGCCGTG
AAAGAGTTTCTGATGTCTCCCAAATACACGAAAGATAACTTTTACGAATG
TGTAGAAACACTTTTCGGTGTCAGGTATATGGGTAAGGGCCTTTTGACTG
ACAGGGACTACGAGCACTGGCACAAGCAGCGTAGGATCATGGACCCTGCA
TTTAGTCGTAATTATCTGATAGGTCTAATGGGGACATTCAACGAAATTGC
AGAAGACCTGGTGGACATCTTGGGCGACAAGGCAGACGGCAAGTGTCAGG
TTGGTATGCATGATATGATGGGAAGAGTCACGTTAGATATTATCGCCAAA
GTAGCATTTGGAATGGAGCTTAACTCCTTACACGACGATCAGACGCCGTT
CACCCGTGCAATCACTACAGTTATGAGGGGAATGGTAGAAACGAGAAACC
CTTTGGCCCGTTATATCCCTGGTAAACAGGCACTTATACGTGATATCAAG
GAGTCACTGAAATTTTTACGTAAAACGGGGAGGGAGTGTATTCTACAGAG
AAGAAAGGCAATCCAAGACGGAGAAGACATACCAAGAGATATCCTGACAC
AAATCCTAAAAGGCGCTGAGACCGAGGGAGACTGCTCCTTGGAAAACCTG
ATAGACAACTTTGTAACTTTCTTCATTGCCGGCCAGGAGACGACAGCCAA
TCAATTGAGCTTCGCAGTTATGGAGTTAGGTAGACACCCTGAGATTTTGA
CACGTGTGCAGGCGGAAGTAGATGAGGTATTAGGTAGCAAGAGGGATATT
GAATACGAAGACCTAGGTAAGTTGCAGTATCTGTCACAGGTATTGAAGGA
GACCTTGAGACTTTATCCCATCGCGCCCGGGACCTCCCGTGCCCTAGAGA
AAGAAATGGTTATTGAGGGCGTTCGTGTTCCACCCGGGACTACGTTAATG
TTTAATGCATATATAATGGGAAGGATGGAGAAATACTACCATGACCCTCT
AGTCTTTAATCCCGATAGGTTTCATCCAGATGCCCCGAAAATATCTTATG
CTTATCTACCATTCAGCCTAGGACCCAGATCTTGTATTGGTCAAGTTTTT
GCACAGATGGAGGCGAAAGTAGTTATGGCAAAACTATTACAGAGGTTTGA
GTTCGAGTTAGTCGAGGGACAGAGCTTCAGAATATTGGACACGGGTACAC
TTCGTCCCCTTGATGGTGTCATCTGTAGGCTAAGACCGAGAGCAGAGCAT
AAGTCCAGGAAG

Seq. ID No: 200

>T5H_8ATGTGGACAATCCTTCTTTCTACCATCAACATAACGCTGGCAAC
AGCTCTAATGTTATCCTTCATAATAATCTACCTTCTATATATCCAAAATT
CAACAAAACTGCCCCCTGGCCCCACCTCTTGGCCTTTAATTGGCTATACT
AGCTGCCTTGGCACTGATGCGTTCAGGAAGATTCAAGATCTTAACAAGAT
ATATGGCGACATAGTCAGCTTCCAAGTATTAGGGAAGACTATAATTATAT
TATACAATTATGATTTAATCCATGAAGCTGCCAATGGCAATAGGTCTAAG
GTGGGGCGTTACACCATGACCGTGAATGATTTGCTGGCGGAAAATAGCGG
CATCTCTAATTATGACACGCAGAAGGCACTTGAAATGCGTAAAGCGTTCG
TTAGGCTTGTCCACAACAATATCAAGACTACGGAGGAACATGAGGGCAAC
AAGCTACAACCGTTTATTAGCCAAAATATAATAAACGCACAGATCAATAA
GCTGATTAGGCAGCTGAGAATCAGGCAGGGCAAGCCGGTTAACGTTTTAC
AGCTTATGAGGTGCACCGTCTGGAGAATCATTTGGAATTTGATATTTGGC
AAGGAGTGCCAACTGACTGATAAGCAAATCTCAGACACACTGGACGATAT
TTCCAGCAATAACCTGCAGAACCAGCTATTTCAAATCAGGCAGTTGCTGC
CAAGGTTTTGTGTTAACATATTCAAGCACAGCCAGTTCGCCCGTAAGCTA
TTCGAGATCGAGGAAATAATCTACAAGTATAAAACTGTGAGACAATTAAT
TGATAATAATGTTGGAGAGATGCATAACTCCGATAGCTTGCTGGGTCAAT
TAATAAACGATTTGAAACTGAATCTAACAAAGAATGACATCTCCCGTCTA
AGTTTTGAATTTATGGCAGCTGGAACCGATACTACCTCTCTAACCTTGAC
GTGGGCATGTGACTATTTAGCAAGAGCCCCGCCAAAGGAGTCACTGAAAT
TATCCTCCGACCTGATTGATATGATCCATAGATGGGCCTCAGTTGTGCCT
CTTTCTTTGCCTCACATAGTTCGTGAAAGTTTCAAGCTAAAGAACTATTA
TATCCCGAAATCAAGCATATTAATATATAACTTGTACGCCGTCCACAATT
CCCAAATTAAGAAGCTAATCAATACGGAGCAAAATTCTGATGAGATTCAG
GAATCCGATAAGCCAATCCCCTTCTCTCTTGGTAGTAGATCATGTCCGGG
GGCTCGTATAGCAAATTTATTGATCGAACAAATTCTGACAGCCATTAACC
AGGAGTTCCTTATTCAAAATATAACGCAGAGCCCGTTTGAGACCATTTCC
CCAGGAAACCAGGAAAGTTTAACGCCTTTCGGGATTACTAGGACTCCACA
TAAAAGTATGTATATATTCGTAACGAAACTAAATGGAAATCGTAGGACTT
CAATT

Seq. ID No: 201

>T5H_9ATGTCCCAGTTGCTATCGAGCTTGATAGAGCTACCTACTCAGAC
GCTGGTGCTTGCAACAGCAGTGGCTGTAGGGGCAGCCGCCTTATTAGTCC
ACGCGTATCTTTTCGACGCCGTTGGGAAGCATGGAAACCTACCGCCGGGC
CCACCAGTTGATTCACTATTTTCTGGACATAGAATACCGTCAACGCATCC
TTGGAGGTACCTGGAGAAGCTAACAGAAGAATATGGCGATATTTTTACCC
TGAGGATTGGTAGATCACCTTTATTTGTCCTTGGAAGAGCTAGTTCAGCT
CATCGTATTCTGGAGAAACAAAGTGCGCTGAGCAGCTCCAGACCTCGTCT
AGTTCTTGCCGGTGAACTATTGAGTAATAATAAAAGAATATTACTTATGC
CTTATGGAGACCAGTGGAGGCTGTATAGAAAAGCCATGCACGAAACATTG
AACGACACGGTGGCGAAACAATATGAACCTATCCAAGAGAGGGAGGCTAG
AATTGCGACGTTACACCTAGGAAGGCTGGGTCAGGCCGATGGCGGTGGAG
GTGACTTCCAGCGTGTCTTGCACAGGTACGCGGCCAGCGTAATAATGCAA
GTTACGTATGATTACCAGGTTCAGACTCTGGACGATCCCCTGGTGAGGTC
AGTGGCCCAGAGGGGTCACGCGTTGGCGATGTGCATCCGTCCTGGCGCTT
CTGTTCTTGACAGGTATCCACTTTTAGAGCACGTTCCCACGTGGTTGAAT
CCCTGGAAACAGGAAGGTTTGAGGCTAAGAAAACTAGAGCAAGAGCTATA
TCTAGGCCAAGTGATCAAGGTCAGAGAAAGAATGGAGAGAGGGGAGTGTG
CCCCGTGCTTTGTTTCTAAAATGACTGAGCGTCAGCAAGAACTAGGGCTT
ACCGATCTGGATGTTGCGGGGATGAGTGGGAGTCTGTTCGGTGCTGGGTC
AGATACAACGGCGAGCGCACTTTCCATCTTCGTGATGGCTGTGTGCAGAT
ATCCCGCAGTGTTAGCCAGACTTCACGAGGAATTAGACAGGGTGGTAAGC
CGTGATCGTATGCCCACCTTTGATGACATTCCGCAAATGCCCTATGTGCG
TGCCACAGTGCAAGAAGTACTAAGATGGAGGCCAGTCTCTGCCGGTGGTT
TCCAACACAGCCTGACAGCGGATGTAGAGTACAAAGGCTACGTTTTGCCT
AAGGGGTCCACGGTGGTGGGCCCCCATTGGTCAATCAGCAGGGACGAGCA
CGAATATCCTGAACACGATGTCTTTAAACCTGAAAGGTTTTTACAAAGCG
GGGGAGCGGAGGCTAACGGCACTTCAGCCCAGGACGAAGTAAAAGGGACT
TGGTTCGCGCCCGCACGTGGCTCTGTCGCATTTGGATTTGGGAGAAGAGT
TTGTCCAGGGTTAAACGTTGCCATGCGTTCTTTACACATTAACATTGCTT
GTATGGCATGGGCTTTTGACATTGCCCAGCCCGATGGCAGACCGGAGAGG
GTCGATACGTTTGCGTTCAACTCTGCAGCGAACTCACATCCTCTACCATT
TGACGCTACCTTCACGTACAGGGACCCCGCGAGGAAAGGCGTAGTCGAGG
AAGAGAATATTGCTACAGGGGAGTTAGACAGGATCGCTGCATCAAGAGGT
GCAACT

Seq. ID No: 202

>T5H_10ATGTTAGAAGCATTGTCCAGTCTAGCTACAGCACTGTGGGCTG
CTTTGAGACCTGATACCGTGCTTCTAGGGACACTGGCTTTTCTGCTATTT
GTTGACTTCTTAAAAAGAAGGCATCCTAAAAACTATCCACCAGGGCCCCC
TGGTCTACCGTTCGTGGGAAACCTGTTCCAACTAGATCCCGAAAAGGTGC
CATTAGTCCTACACCAGTTTGTGAAAAAGTATGGGAATGTCTTCTCCTTA
GACTTTGGAACCGTCCCATCTGTGCTAATAACCGGCTTGCCGTTGATAAA
AGAAGTGCTAGTTCATCAAGGACAAATCTTCTCTAACAGGCCTATAGTCC
CGCTACAAGAACATATCATTAATAACAAAGGCTTAATTATGTCATCAGGC
CAATTATGGAAAGAACAACGTCGTTTTGCATTAACGACACTGAGAAATTT
TGGTTTAGGGAAAAAGTCACTTGAAGAGAGAATCCAAGAAGAGGCCAGCT
ATTTAATCCAGACAATCAGAGAAGAAAACGGACAGCCTTTTGATCCTCAT
CTGACGATTAATAATGCGGTTTCAAACATTATTTGTAGTATTACCTTCGG
GGAAAGGTTTGACTACCAAGATGACCAATTTCAGGAGCTGCTAAGGATGT
TGGATGAGATACTAAACCTTCAGACATCCATGTGCTGTCAACTTTACAAT
GTCTTTCCGAGAATAATGAACTTCCTGCCAGGACCACATCAGGCGCTTTT
CAGTAACATGGAGAAAATGAAGATGTTTGTCGCAAGAATGATCGAGAATC
ACAAGAGAGACTGGAATCCTGCTGAGGCCCGTGATTTCATTGACGCCTAT
CTACAGGAGATTGAAAAGCACAAGGGCGACGCCACATCCAGTTTTCAAGA
AGAGAATCTAATTTATAATACACTTGATCTGTTTCTTGCGGGGACCGAGA
CTACGTCAACTAGCCTACGTTGGGGGCTACTGTTCATGGCTTTGAACCCG
GAAATACAAGAAAAAGTTCAAGCAGAAATCGATCGTGTCCTGGGCCAGTC
CCAACAGCCGTCAATGGCTGCCAGAGAAAGCATGCCCTATACTAACGCCG
TAATACACGAAGTATTGAGGATGGGCAACATAATCCCCCTAAACGTCCCC
AGAGAGGTTGCCGTAGATACTACCCTGGCGGGATACCACTTACCCAAAGG
TACGATGGTTATGACTAATTTAACTGCGCTTCACCGTGACCCCACAGAAT
GGGCTACACCCGATACTTTTAATCCCGAGCATTTTCTTGAGAATGGGCAG
TTCAAGAAGAGAGAATCTTTCCTGCCCTTCTCCATAGGGAAAAGAATGTG
CCTTGGAGAACAACTGGCAAGGACCGAACTATTCATCTTTTTCACAAGTT
TACTGCAGAAATTCACTTTTCGTCCGCCTGAAAACGAGCAGTTGAGCTTA
AAATTTAGAGTATCTCTTACCCTAGCCCCGGTAAGCCACAGGCTGTGTGC
GGTCCCTCGTGGG

Seq. ID No: 203

>T5H_11ATGAAGACCCCGCCCCAGTCCTCATGCCCTTTTCATGCGGTTG
GACGTCCTCCCACACCCCCTAGATCATCTGCGGGCAGATGGCCCCCAGGA
CCAGAGTCAGGTCTGACGGGTTGGGGGTTATTAAAGCTGATGAGCAGAGA
TTTAATGGGAACGCTTGCAGGGTGGCAAAGGGAGTTTGGCGATCTTGTGC
ATGTCAGGACCTGGCCTGAGCATCAAGTCATAGTGTCCGATCCACAACTA
GCGAGAGAACTTCTAGTCAATCAAGCCGACGCATTACAAAGGTGGGAGCG
TGCGCTTACAGTATATCGTAGGGTTCACGGACACAGCGTCCTGATAGCCG
AGGGCCAGGCCTGGAGGGAGAAGAGACAAGCCCTTCAGCCCGACTTTACA
AGGAAATCAGTACAGGCCTTTAGTCCCAGTATCGTAGAAGCAGCGCGTAG
GGCGTTCGAACAGTGGCCTGCGAGGCATGCTGCTTGGCCAATTGAATCCG
AGCTTACCTCAGTTACGATGGAGGTGATACTTCGTATGATGTTTAGCAGT
GGAGTGGGTTCTGAAGCCCAGCAAGCTGAGGAAGCTGTACATACCCTAAT
GGTGGCATCAACCGAAGAGCTTTGGAGACCCGCCAGCTTGCCTGATTGGG
TTCCCTGGCAAAGGAAAAGAAGGCGTGCGAGACTACTAATGAACGGTCTA
ATTGAAAGGCACTTGCAGGCGCGACTAGCAATGCCTCAGGATGCTTGGCC
CGAGGACTTGTTGAGTCGTTTACTTCGTCTTCACTTGCAACAGCCCCAAT
CCTGGCCACTACAGGCTGTGAGGGATGAGTGCAAAACAGCATTCTTGGCT
GGGCACGAAACTGTCGCAACATCACTTACATGGTGGGCTTGGTGCATGGC
GTCTCATCCGGAAATCCAGGAGAGGGCTCGTGAAGAGGCCCTAGCGGCCT
TGTCTGGTGGAGGACAAGCAGACCCAGCTGCCCTACAATACGTCAATCAA
ACATTGCTAGAAACTATGCGTCTATACCCTGCCGTCCCCTTACTTATGAG
TAGAAGAGCACTGAAACCAGTGACTCTGGGCGACTGGACTTTTCCTGCGA
AGACTGTCTTCATGGTTCCAATGCAGCTTATGCAGCATGACGAGCGTTGG
TTCCCGGAACCGAGATCTTATCGTCCAGAAAGATTTGGGCCTGATGCTGC
CCGTCCACAACAGGGAGCGTATTTGCCATTCGGAGGGGGCCCTCGTGTGT
GTTTAGGACAACATCTAGCTATGGCGGAAATGGCGTTGGTGGCAGCACAG
CTGCTTCTTAGGTATAGGCTGTCTGCGCCAGAAGGCGCGGAACCCCCTAG
ACCCGTTTTCCACGTCTCACAAAGACCAAGCCAGCCACTGACTCTAGGTA
TTGCCCGTATT

Seq. ID No: 204

>T5H_12ATGAAACTTGCAGGTAAAAGGTTCAGGCTACCCCCTGGGCCAT
CAGGAGCGCCTATTGTCGGCAACTGGCTTCAAGTGGGGGATGATCTAAAT
CACAGAAATCTAATGGGTCTAGCTAAGAGATTTGGAGAAGTGTTCCTTTT
GAGGATGGGTGTGAGGAATCTAGTTGTCGTGTCTTCACCAGAACTTGCTA
AAGAGGTTCTGCACACCCAAGGAGTTGAGTTTGGTTCAAGGACGAGGAAT
GTAGTGTTTGATATTTTTACGGGCAAAGGACAAGATATGGTCTTCACCGT
TTACGGGGACCACTGGCGTAAGATGCGTCGTATAATGACGGTGCCGTTCT
TTACCAATAAGGTAGTTGCGCAAAATAGAGTCGGTTGGGAAGAGGAAGCC
AGACTTGTGGTGGAAGATTTGAGGGCGGACCCTGCAGCAGCAACCAAAGG
GGTAGTAGTCAGAAGGCGTTTGCAACTTATGATGTACAACGATATGTTTA
GGATCATGTTCGACAGAAGGTTTGAAACCGTGGCAGACCCCCTGTTCAAC
CAACTTAAAGCACTCAATGCGGAGAGGTCCATCCTTAGTCAGAGCTTCGA
TTACAACTATGGAGATTTTATTCCGGTGCTGCGTCCCTTTCTGAGACGTT
ACTTAAACAGGTGTACTAATTTAAAAACGAAGAGGATGAAGGTCTTCGAA
GATCACTTTGTCCAACAGAGAAAAGAAGCCCTAGAGAAAACTGGCGAGAT
CAAGTGTGCAATGGATCACATATTGGAAGCAGAGCGTAAAGGTGAAATTA
ATCATGATAATGTCCTTTATATTGTGGAAAACATCAATGTTGCGGCGATC
GAGACGACACTATGGTCTATTGAGTGGGGTCTAGCAGAGCTGGTCAATCA
CCCCGAAATACAGCAGAAACTTAGAGAGGAAATTGTAGCGGTGCTAGGTC
CAGGTACACCAGTAACGGAGCCCGATTTGGAACGTCTGCCTTATTTGCAG
TCCGTAGTAAAAGAAACACTGAGGTTACGTATGGCTATTCCTTTACTGGT
CCCTCACATGAACTTGAGCGATGCGAAATTAGCCGGGTATGATATTCCGG
CGGAATCAAAGATCCTGGTGAATGCCTGGTTCTTGGCAAACGACCCAAAG
AGGTGGGTCAGAGCGGATGAGTTTAGACCCGAAAGATTCTTGGAGGAGGA
AAAGAGCGTGGAAGCACATGGGAACGATTTCCGTTTTGTCCCCTTCGGCG
TTGGCAGAAGGAGCTGTCCAGGCATAATATTAGCCTTGCCGATTATTGGT
ATTACCCTGGGTAGGTTGGTACAGAATTTTGAGCTGTTACCGCCACCTGG
CCAGGACAAAATTGATACTACGGAAAAGCCGGGTCAATTCTCCAACCAGA
TCTTAAAACACGCAACAATAGTATGCAAGCCGCTAGAGGCT

Seq. ID No: 205

>T5H_13ATGCACACGGATACCCCTGACACGACCGCCGATCAACCGCTTA
GGCGTATAAAAGATTTGCCAGGACCTAGACCTTTGCCCCTTATAGGCAAC
GGTCATCAGATAAAACCCCAGAGGATTCACCAGCATGTAGAGAGATGGAG
TCTTCAGTACGGGCCCCTAATGAGGATGTACTTCGGAGCGACTCCCATTT
TGGTCGTCGCAGATCACGAAATGGTAGGTGCTGTCTTACGTGATAGACCT
GACGGGTTTCGTCGTCCCAGTATAAGTGCTACGATTTCAAATGAGATGGG
AGGCATACCGGGCTTATTCCTGGCAGAAGGAGCAGACTGGCGTAATCAGA
GACGTATGGTTATGGCAGGCTTCGCGCCCACGGCAATAAAAGCGTATTTT
CCTGCACTGGTCGCGGTAGCTCTAAGGCTAAGAAGACGTTGGCAAGCAGC
AGCATCCGCAAGAAAGGCTATCGATCTTGAGAGCGACCTGAAGAGGTACA
CAGTTGATATAATAGCTGGATTGGCCTTCGGGTCAGATGTTAATACCCTG
GAGTCTGGAGAAGATGTGATTCAAAGACATCTTGATGACATCCTTCCGGC
AGTCGCACGTCGTTCTTTGGCATTAGTACCCTATTGGCGTTACGTGAAAC
TTCCGGCCGATCGTAGATTAGATAGGTCAGTGGCCGTGCTAAGAACTGCC
GTTCAGGACCTTATTGGGCAGGCTCGTCAGAGAATGCTTGATAACCCGGC
CAGACGTGAGAGGCCCCCGAATCTTTTGGAGGCTATGATAGCTGCCGCGG
ACCAGAGCGGTAGTGGAGTTACCGATTTGAACGTTGCTGGGAACGTCACC
AATATGTTATTAGCTGGAGAGGACACTACTGCGAACACAATATCCTGGAT
GATCTACCTTCTGCAGCGTCACCCTCATACCTTGCAAAAAGCTAGAGACG
AAGTTAGGAGAAACGCCCCTGACGCAGCCAGATTTACGATCGAGCAACTA
GATTCTTTGGACTACTTAGGCGCCTGTGCGAACGAGGCCATGAGACTTAA
ACCTGTTGCGCCTTATTTGCCTCTGGAGGCTCTAAGGGATACAGTAATTG
GAGATGTTGCGGTGCCTGCGGGTACCATGATCTGGTGTGTGTTGAGACAT
GACAGTGTAGCAGAAAAGCACTTCCCCGATCCACTGTTGTTCGACCCACA
GAGATGGCTGCAGGCTGATGGAAAGCCGAACTCCGATAAGCGTGTTACAA
TGCCCTTCGGTGCCGGACTAAGAACTTGTCCAGGCAGATATCTGGCTTTA
CTAGAGATCAAAATCGCAATGGCTATGTTGCTGGGAAGTTTTGATATTGC
CGGGGTGGACACGCCGGACGGCAAGGAGGCTCAGGAGCTTATGGGGTTTG
TGATGTCTCCAGTTGGATTATCTTTGAGATTAGAG

Seq. ID No: 206

>T5H_14ATGCTGATGAAAACCTTAATGGCATCACTTCAATGGTTGAAGG
AATCCTTCCAGCCGTTTATGTTACTGTTTGCTTCCATTTTTCTTGCCGTG
CTTCTAAAGTTCTTCTTCAAGGAGAAGAGCCGTAAAAGGTCAAACTTACC
TCCGAGCCCACCAAAGTTACCGATTATTGGGAACTTGCATCAGCTTGGCA
ACATGCCTCACCTTTCCCTGCATAACTTGGCAAAGAAATATGGGTCAATT
ATCTTTCTTCAACTGGGGGAAATTCCCACCGTGGTAGTCAGTTCAGCTAG
GCTTGCGAAAGAGGTAATGAAAACTCATGACCTTGCTCTGAGTTCTAGGC
CGCAAATTTTCTCAGCGAAGCATCTTTTCTACAACTGCACAGATGTTGTA
TTCAGTCCGTACGGCGCTTACTGGCGTCACATTAGGAAGATCTGTATCCT
GGAGTTACTGTCAGTTAAGAGGGTCCAGAGTTACTCTTTTGTGAGGGAGG
AAGAAGTGGCTCGTTTAGTCAGGAGAGTCGCAGAGTTTTACCCAGGCACT
ACGGACTTGACTAAGATACTGGGATTATACGCAAATGACGTCCTTTGCCG
TGTAGCCTTTGGCAGAGATTTCAGTGGGGGCGGAGAGTATGATCAGCACG
GGTTCCAGAAAATGCTGGAGGAGTACCAGGAATTGTTGGGCGGTTTCTCT
CTAGGTGACTTTTTCCCCAGTATGGAGTTCGTGCACAGTTTAACCGGGAT
GAAGAGTCGTTTGCAAGACACGTTTCGTAGGTTCGACCAGCTATTCGACC
TTTTCCTGACGGAACACAGAGACCCAAAGAGAGAGACAGAAGAACACAAA
GACCTGGTGGACGTGTTATTAGACTTACAAAAGAATGCCTACGATGAAAT
GCCGCTGACAACGGACAATATCAAAGCTATAATATTGGACATGTTTGCGG
CAGGGACCGATACTACGTTTATCACTTTAGATTGGGGGATGACTGAACTA
ATCATGAACCCCGAGGTAATGGAGCGTGCCCAGGCCGAGGTCAGGTCTGT
GGTCGGTGACCGTGAGGTTGTATTACAGAGTGATCTGCCTCAGCTACATT
ACATTAAGGCCGTCATCAAAGAGATCTTTAGACTACATCCCCCGGCACCC
GTTTTGGTGCCCAGGGAATCTATGGAAGACGTTAGTATAGATGGTTATAA
TATCCCTAGCAAAACCAGGTTCTTCGTTAATGCGTGGGCTATTGGGCGGG
ACCCAGAGTCCTGGGAAAACCCCAACGCTTTCGAACCAGAAAGATTTATG
GACAGTACAATAGATTTCAAAGGTCAGCACTTTGAACTTATTCCCTTTGG
TGCAGGTAGGCGTAGTTGCCCCGCAATCGCCTTCGGCGAAGCGACTATCG
AATTAGCCCTAGCGCAACTTTTGCATAGCTTTGACTGGGAGCTGCCTCCA
GGTACGACGCCCAAAGATCTTGACATGTCAGAAGTGTTTGGAATCACTAT
GCACCGTATTGCCCATTTAATAGTCATCGCGAAACCGAGATTTCCCGTCG
GCCAAAACAAA

Seq. ID No: 207

>T5H_15ATGCCCAAACAGAAGAAACGTTTGCCGCCAGGGCCTCCAACAC
TACCAATCATTGGGAACATGCACCAGCTGGGAGAACTTGCGCACAAATCT
CTGTCCGAGCTGTCAAAGAAGTATGGCCCTATTATGCTTTTAAAAATTGG
TAGTAAAACAATAATCAACATAAGTAGCGCAGAAGCGGCGAGACAGGTAT
TGAAAGTGCATGATTTGGATTGTTGCTCAAGACCGGTAAGCTCCACAGCT
GGCAGACTAACGTACAATTTTAAAGATATTGTATTTGCACCGTATGGCGA
TTACTGGAGGGAGATGAGGAAAATCTGCGCACTAGAGCTGTTGAGCGTGG
CGAGAGTGCAAAGCTATCGTTTCATTAGAGAGGAAGAAGTCGCAAGTCTA
GTAAATTCTATCTCCCAGAGTGCATCTTCTGCCACCCCGGTTGATCTGTC
TGAGAAGATGTTAGCATTGACAGTGAACATACTTTGCAGGACTGCGTTCG
GTAAAAGTTTCAGAGGATCAGGACTTGACAATGGTAAATTAAGAGAGGTC
GTCCACGAGGCTGAAGTCATGTTCGCGAGCTTTTCTGCGACCGAATTTTT
CCCCTACGTAGGTTGGATTATTGATCGTCTAAGCGGACGTATAAGAAGAT
TAGAGAAAATTTTTAGAGGGCTAGACGATTTCCTGCAACAGGCTATTGAT
TTGCACTTAAAACCTAAAAAGACCGAACAAGACCATGAAGATCTAATTGA
CGTACTATTGAAGATAGAGCGTGACCAACAAACCAATACGGGGGCACCCC
CGTTCAACAAGGACAACATCAAGGCCATACTTTTTGATATGTTCTTGGGT
GGATCAAATACAGCCGCAGTGACCATGTTGTGGGCTATGGCTGAACTAGC
AAGGAACCCCAGAGCTATGAAGAAAGCTCAAGATGAGGTACGTAATGTCG
TAGGCAATCGTGGAAAAGTCACTGAAAGTGACATCACACATTTGCATTAC
CTGAAGATGACCATCAAAGAGACGTTTAGGTTACACCCGCCCGCAGCGAT
ACTTCTTCCAAGGCAAACTATGGCCGAGGTCAAGATCGGGGGCTATGACA
TCGGCCCGAACAGCCTTTTACAGGTTAATGCGTGGGCTCTAGGTAGGGAC
CCGGAGTACTGGATGAACCCCGAAGAGTTCTACCCAGAGAGATTCGTTGA
CTCTAGCATAGATTACAAGGGACAACATTTCGAATTATTACCTTTTGGTT
CAGGTCGTAGGGGCTGCCCGGGTATGCACATGGGAACAACAACGGTCGAG
TTAGCATTGGCTAACTTGCTGTATTGCTTCGACTGGAAGTTGCCCAGTGG
TCTTAAAGAGGAGGATATTAATATGGATGAGTCTACGGGTCCGGGGCTGA
CCCAAAAGAGAACGACACTGAAGTTAGTGCCGGTGAAGTTGTTT

Seq. ID No: 208

>T5H_16ATGAAGCTACTGCTAGATAGGACCAGAACTAATGGGTACCTTC
CCCCTAGCCCTCCAAAGCTACCCATCATTGGTAATTTACATCAATTAGGC
AAAATGCCGCACATCAGCCTGTGTGAGCGTGCTCAGAAGTTGGGACCGAT
TATGTTCTTGCAACTTGGAGAGGTACCGACTGTAGTCATCTCCTCAGCGG
CTATGGCAAAGGAGGTGATGAAAACGCATGATTTAGCTTTCTCAAGTAGA
CCGCAGTTATATTCCGCCAAGTGGTTGTTCTACAATTGCACTAATATCGT
ATTTTCACCTTATGGAGCGTATTGGAGACACGTTAGAAAGATTTGCATTT
TAGAGCTGCTGTCCACTAAAAGGGTCCAGTCTTATGGGTTTATTCGTCAA
GAAGAGGTTTCAAGACTACTTCATAGAATAGCTGATTCTTGCAGCAAGCC
AATAAATCTAAGCAAATTGTTGGGCCTTTACGCGAATGATGTGCTATGCC
GTGCTGTCCTGGGCAGAAATTTTAGTGAAGGCGGGGATTATGATATGCAT
GGGTTCCAGTCTATGTTGAAAGAGTACCAAGAGTTGTTGGGTGGCTTCAG
TATAGGAGATTTCTTTCCAAGTAAAGAATTTGTCCATTTATTAACAGGCC
ACAAAAGGAGGTTACAAAATACGTTTAAAAGATTCGATAACTTCTTTCAA
CAGGTTGTGCGTGAGCATTTAGATCCGGAAAGAAACTACGAAGGTGAAAA
AGATATCTTAGATGTCTTGTTAGATATCCAAAAGAATGGAAGTAGCGAAA
TGCCCTTAACACTGGATAATGTTAAAGCCATTCTGTTGGATATGTTTGCA
GCCGGAACAGACACATCATTCATCGTCCTGGACTGGGGAATGACCGAACT
AATCATGAACCCAAAGGTTATGAAAAAGGCGCAAGCAGAAATAAGGAGAG
TAGTAGGTGAGAGGCAAGTCGTCCTTGAAAATGATCTGCCGCAACTACAT
TATTTAAAAGCAGTCATTAAAGAAATATTCAGGCTGCATCCGCCTGTCCC
GGTACTGGTCCCGCGTGAATCTATACAAGATGTGACCATTGAGGGCTACA
ATATCCCAGCAAAGACAAGGGTGTTTATCAACGTGTGGGCAATAGGCCGT
GATCCAGAAAGCTGGAAGAACCCAGAGACGTTCGATCCGGAACGTTTCGT
TGGTAGCACAATCGACTTTAAAGGCCAGGACTTCGAACTGCTGCCCTTTG
GGGCTGGCAGGAGGGGATGTCCAGGAATTACATTCGGAGCAGTAACTGTT
GAATTGGCCTTAGCGCAACTGTTGCATAGCTTCGATTGGAAATTGCCTTT
AGGGGTTGAAGCAAAGGACCTAGATCTAACGGAGGCATTTGGAATTTCCA
TGCCTAAGACGAGTGACTTGATCGTCGTCGCCAAGCCGTGTTTCGCA

Seq. ID No: 209

>T5H_17ATGAGAATGGACGGAAATTCAACTACGATGTTCCCTCTTCTGA
TAACGGTCATAATGCTTTTGGCCTCTGTGTTATTTTATATATTCAACAGG
TGGACTCATAGGTATAGTAAGTCAGGAATACTGCCTCCTAGTCCGCCTAA
GTTACCCCTGTTAGGTCACCTGCACTTGCTTTCAGATCAACCGCACGTTG
CCCTGTCAAGGTTAGCTCAAAAGTATGGCCCAATTATGTACTTAGAGCTG
GGACAAGTGCCGACTGTGGTTGTTAGCTCAGCGTCCTTGGCACGTGAAGT
CCTTAAAACCCACGACCATGTATTTTGTAATCGTCCGCAAACTATAGCCG
CACAGTACATATCATTCGGGTGTTCAGACGTGACTTTTTCTCCTTATGGG
CCGTATTGGAGACAGGTGAGAAAAATATGCGTCACAGAATTATTGACCCT
GAGGAGGGTTAACTCTTTTCAGTTAATACGTGAAGAGGAAACTAATAGGT
TGCTTACGGCCGTCGGAGCGCACTCAGGGAGTGAGGTGAACTTAACGAAA
TTATTTTTCAATTTGGCCAATGATACGCTATGCAGAGCCGCATTCGGAAC
GAGATTCATGAGCGAGTCTACCCAGTTAGAAAGGCAAAGGGAGGGCAAAA
GACTTGAGGATATTCTGATTGAGACCGTAAAACTGTTAAGTGGATTCTAT
GTGGGCGAGTTCTTTCCCAGGTGGGGGTGGATTAACAGCGTCAGCGGTTT
CAAACGTAGACTTGAGAGAAACTTAGCAGATTTGCGTTCAGTAGGTGATG
AGATAATCCAAGAACACATTAAGAAGAGAGGACGTGGTAACGAAGAAGAG
GATTTTGTAGACGTATTGTTACGTGTCCAACGTCAACAAGACTTGCAAGT
CCCAATCACTGATGACAACGTAACGGCACTTGTTATGGATCTTTTCGTGG
CGGGGACAGATACCACATCCTCCACTTTGGAGTGGACGATGACCGAAATG
GCCAGGCACCCGGAGGTGATGAAAAAGGCTCAAGCAGAAGTGAGATCTAT
GTCACCCGAAGGGGGCACGTTGGACGAGTCCCATTTAAGACATTTGCACT
ATCTTAAAGCTGTTATTAAAGAGGCACTTAGATTACATCCACCAATACCT
CTACTTCTGCCCCGTGAAAGCATGGATAAGTGTGCCATCGACGGTTATGA
AATCCCTGCTAAGACAAGAGTACTTATAAATAACTTCGCACTTGGAAGGG
ACCCCGATTCTTGGGACGACCCTCTTCGTTATAACCCTGCCCGTTTTATG
GGTGGCGATGAACATAAAATCGATTTCAAAGGGGAAGATTTCAGATTTGT
TCCGTTTGGCGGTGGTAGACGTGGGTGTCCCGGCTACTCCCTGGGACTTG
CTACCGTTGAATTGACCCTAGCCCGTTTACTATACCATTTTGACTGGAAG
TTGCCGCCTGGAGTAGAGGCAGAGAAAATTGATTTGACAGAAATTTTCGG
CCTAGCTACTAGGAAGAAAACCCCATTACTGCTTATTCCGACTGCCAGGA
AAGCTCCACCGCATGAA

Seq. ID No: 210

>T5H_18ATGGAATTAACTATGGCTTCTACCATGTCTCTGGCATTGCTAG
TTTTAAGCGCCGCATATGTCTTAGTCGCTCTAAGAAGATCCCGTAGTAGT
TCCTCAAAACCTCGTAGACTTCCACCATCACCTCCCGGTTGGCCGGTTAT
TGGACACCTGCATTTGATGAGTGGGATGCCACACCACGCGCTTGCGGAAT
TAGCAAGAACCATGAGGGCTCCACTATTTAGAATGCGTTTAGGTTCCGTG
CCAGCTGTTGTAATATCAAAACCCGACTTGGCTCGTGCTGCACTTACCAC
AAACGATGCGGCATTGGCGTCCCGTCCGCATCTGCTTTCAGGACAGTTTC
TGAGCTTCGGCTGCAGCGATGTAACTTTCGCACCGGCAGGACCGTATCAT
AGAATGGCTCGTAGGGTGGTTGTAAGCGAGTTACTAAGCGCACGTCGTGT
GGCAACATATGGGGCGGTGAGAGTTAAAGAGCTTCGTAGGCTATTGGCCC
ATTTGACGAAAAATACAAGCCCTGCTAAGCCTGTAGATTTATCAGAGTGT
TTTCTGAATTTAGCCAATGACGTGCTATGTAGAGTAGCTTTTGGTAGACG
TTTCCCACACGGAGAAGGGGACAAGCTGGGAGCCGTACTGGCTGAAGCGC
AAGATCTATTCGCAGGCTTCACTATAGGGGATTTCTTCCCGGAATTGGAG
CCTGTAGCTAGTACGGTTACTGGGCTACGTCGTAGATTAAAGAAATGCCT
AGCCGATCTTAGGGAGGCGTGTGACGTGATAGTAGACGAGCACATTTCCG
GTAACAGGCAAAGAATCCCAGGTGACCGTGACGAGGACTTCGTTGATGTA
TTACTACGTGTCCAAAAGAGCCCCGACCTGGAAGTACCACTTACGGACGA
CAATCTGAAGGCCTTAGTGTTAGATATGTTCGTGGCAGGTACGGATACCA
CTTTCGCAACACTGGAGTGGGTCATGACAGAGCTTGTACGTCATCCGAGA
ATCCTAAAGAAAGCTCAGGAGGAAGTTCGTAGAGTGGTCGGCGACTCTGG
TCGTGTTGAGGAAAGCCATTTAGGAGAACTGCATTACATGAGGGCCATTA
TTAAAGAAACTTTTAGACTTCACCCAGCGGTTCCCCTATTGGTACCGAGG
GAGTCTGTTGCACCATGTACTTTGGGTGGGTATGACATACCTGCAAGGAC
GAGAGTATTCATCAACACGTTTGCGATGGGGAGGGACCCGGAAATTTGGG
ATAATCCTCTTGAGTATTCTCCAGAAAGATTTGAGTCTGCCGGCGGGGGA
GGCGAGATTGATCTCAAGGACCCCGATTACAAACTTCTACCATTCGGCGG
CGGAAGACGTGGCTGTCCAGGATATACGTTCGCCCTTGCGACCGTTCAGG
TTTCATTGGCTAGTTTATTATATCACTTTGAATGGGCACTACCGGCAGGG
GTAAGAGCCGAGGACGTCAACCTTGACGAAACTTTCGGTCTAGCAACTAG
AAAGAAAGAGCCCCTGTTTGTTGCGGTACGTAAGAGTGATGCTTACGAGT
TCAAAGGAGAGGAGCTAAGTGAAGTC

Seq. ID No: 211

>T5H_19ATGCCCCTAAGCGACAGCACTATCTCCCTTCTACTGGCCGTGT
TGCCGATTAGCGGGATCATTTTCGCCCTATATAACCAGTACCAGATTTGG
CTTAAATCTCCGATAAGAGGGCTGCCCTATCCCCCTGGACCACCCTTGTT
GTTGGGGAACGCGAACAGGGCTGTACAGTCTAGGCCCTGGTTAACGTATA
CAGAGTGGGCCAAGCAGTACGGAGACATAATATATGTCAATATATACGGA
GAACACACGGTCATTTTGAATAACTTAGAGGATGTTATGGAACTGTTCGA
ACAGAGATCAAGGGTTTATTCTTCCAGACAAAACAACCCATACATCGAGC
TGATGGGATGGCAGTTTAACGCTGGGCTTCTGCCCTATGGTGATCTGTGG
AGGAGACATAGGAAGTTACTACAGCAGTGTTTCAGAAGAAAGATTTCCAC
ACAGTACGAACCCATACAGATAGCTAAGACCCACAACCTACTGAATGATT
TACTGCAAACACCTAGCGACTTCATAGAGCATATCAAACGTAACAGTTCT
GCCATGATAATGAGTATCCTATACGGTCAAGACATCTCTGACGAAATGTC
TGCCCAATTCGTTTCTGTGGCTGAAGAAAGTGTAAAGGCTCTGGGGAAGT
GCCTGCGTCCCGGCACCTACCTTGTATCTTACATCCCAATGTTGCGTTAC
CTGCCAGCCTGGTTTCCCGGAGCAGAATTTCAACGTCAAGCTGCTGAGGT
GAAAAAGCTAACTACCAAAATGAAAGATGAGCCAATTGATTTCGTAGGGA
AGGGTCTGTTACACGGCACAGCATCAGCATCTCTTGTGGCAGACCTATTG
GAAAACTGCTATGTGCAAAGAGAATACGACGTAATTAAAGACGTAGCCGC
GACCGTTTTTGCAGCGGGGGCAGATACCTCTGTGGCAGCATTGGAGTCCT
TTTTCCTAGCTATGAGTCTTTTCCCGGAAGCTCAGAAAAAGGCCCAGGCT
GAGATGGACAGAGTAATTGGTAATAAAAGATTACCAACGACCGATGACAG
GCCACTGCTACCTTATCTGGAAGCCGTATACCGTGAGCTGATGAGGTGGG
CTCCTGTAGTGCCTCTAAACGCGGCGCACACCACAATCGCGGACGACATC
TACAAAGGCTATTACATACCGAAGGGCACTGCAGTCTACGCTAACACGTG
GGCCTTGACGCGTAATGAGGAAAAGTACCCCAATCCAGACATATTTAATC
CCGATAGGTTCTTTACCGAGACCGGAGAGCTAAATGACGATGATACGGTA
TTGACTTTCGGGTTTGGAAGGCGTATATGTCCGGGCAGGCACATGGCATC
TACGACGGTTTGGTTAACTATTGCTTCAGTTTTAAGTAATTTTGATATAA
AGGGGAAAGGCACAAATACGAAAGATCAGAAATTTACTAGCATCGGGGAA
ATGTTTACCGACAACTTCATTTCCAGGCCGGTGCCATTTGAATGCGACAT
TGTGCCGAGAAAGAACGCAGCCCTGCTGGCAAGTAAA

Seq. ID No: 212

>T5H_20ATGGCGTTCGAAACGACGAACGGTATCCTATTAGCTGCCAGTC
TATTCGCGGGGGTGGTTCTATATCTGCAAAAGAGAAAGAGATACACCCTA
CCGTATCCTCCGGGGCCAAAGAAGCACTTCCTTTTGGGCAACTTACTAGA
TGTTCCAACCACGTTTGCATGGAAGAGATACGCAGAGTGGGGGAAGACTT
TTGATTCTGATGTGCTACACTTGAGCGTTGCAGGGAGCCATTTCATCATA
CTTAACTCCTTTAAGGCCGCCAACGATCTGTTCGAAAAACGTAGCTCTAT
TTATTCTTCAAGGGCACAGATGATAATGTTTAGCGAACTGATCGGTTGGG
ATTGGCTGATGAGCGGCATGGTCTATGGGGAGCCTTGGCGTGAAAGGAGG
AAGGCGTTCCAACAATATTTTCACGTAGGCAATGCCCACTTATATGAACC
CGTTCAGATGCAAGCAGTGAGAAAGATGCTACCACGTCTGTTAAAAGAAC
CTGAAGACTTTTTAAGCATCACGAGGCATGCGCTTGGCAGTATGGCACTA
ACCCTAGCCTACGGCCTAGACATACAAGAAAAGAATGATCCCTATTTAAG
AGTCTCTGAAGCTGCAGTCAAGTCTATAGGTGAAGTCGCAATTCCAGGCG
CTTTTCTTGTGGACATGATCCCGGCGCTGAAATATGTACCTGAATTTTTC
CCTGGAGCAGGTTTTAAGAAAAAGGCACGTATTTGGAGAAAAGTCCAGGA
AAACATGCGTGAGATACCTTTCGCTGCTACACTGAAGAATATAGCGAGTG
GTAGCGCCAAGGTCAGTTTTACAAGTACATGCCTAGAAAACCTTGATGAA
TCAAGGGACGTAGACCACCAACGTACTATTATTAAAGATACGGCCGGAAA
TATGTTTGCCGCTGCGACTGATACAACTATTAGTGCTATCCATACCTTCT
TTGTTGCGATGTTGTGCTTTCCCGAGGTCCAGAAAAAGGCTCAACAAGAA
ATTGACAGGGTATTACAAGGTAGGTTACCTGAGTTTAGTGATGAGGCCGA
CTTACCGTACTTATCTGCATTGGTTAAAGAAACGCTGAGGTGGGAGCCTA
GCACCCCTATCGGTGTGCCGCACTACAGTTCTGAAGATGATGTCTATAAC
GGATATCACATACCGAAAGGAAGTCTAGTTATCGGAAATGCGTGGGCAAT
GCTTCACAATGAGGAGGATTACCCGGAGCCTTCACTTTTCAAGCCAGAAA
GGTTCATCAAGGACGGTAAATTGAATCCCAACGTGAGGGACCCGGCCGAA
ATGGCCTTCGGGTTCGGGCGTCGTCTGTGTCCGGGGAACCACATTGCCAT
TTCTGCGCTATGGTTAACCGCTGCAACAGTACTAGCTACCTTTAACATCA
CCGAGGCAATAGATGACGATGGTAGACCGATCAAGCCATGTGTGGAATAT
GAATCTGCGTTAATATGCCACCCCCTTCCTTTTAAATGCACAATCAAACC
TAGATCTAAGGAATGCACAATGTTAATTCAAGCGGCAGCTGACAGCTAT

Seq. ID No: 213

>T5H_21ATGATTATCGATTCTAGTAACTCCGAAGGCAATAGTGAAGGCC
AGTACACCATCGATGGACCTAAGGCCAAAGGACTGAGAAGGATGTTTAGA
ATCTTCCACTTAATCTTACAGCCGACTAAGTATATGGAGTCTTCCGTACA
GAGGTACGGTAGTATGTTTCAGATAGGAAGCGAAGGAGCATCACCATTAG
TATACGTAGGAGAACCAGAAGTTGTGAAAGAGATTTTCGCTTTGGACGGA
GATCAAGTCGTGACAGGGCAGGGTAACGGAGTGCTAGAGACTATGGTTGG
CAAGCACAGTATTCTTTTACTTGATGGCGACCCTCACCGTCAACAAAGAA
AGTTGTTAATGCCTCCATTCCACGGTGAGCAGCTAAGGGCATACGCACAT
TTGATTTGCGATATTACCAGGCAGATCAGTGCACAGTGGCAGCCGGGACA
AACCATCGTGGCTAGACCGCCCATTCAGAATCTTACTCTAGGAGTGATCC
TACAGGCAGTGTTTGGGGTCCCCTCTGGCGAAAGGTTGTCCCGTCTTCAA
CAACTTATGTCCACGCTGTTAGACTCTTTTGCCTACCCCATATCAGCATC
ATTTTTATTCTTCCCGGCGTTACAAAAAGACTTAGGAGAGTGGTCCCCGT
GGGGTAAGTTTATTAGGCTGAGAGAGGAAGTCCGTAGTCTTATTTATGCG
GAAATTAGGGATAGAAGGCAGCAGTTAGAGAGAAGCGCAATTGAGCAAGA
CGAAAAACTTGGAGAGAAGTTAGGCGAGAAGACAGATATATTAACCTTGC
TACTACAAGCGCGTGACGAGGACGGAGGGGCGATGTCTGATGCTGAGCTT
CACGATGAGATAGTAACCCTTTTGCTTGCCGGCCATGAAACGACAGCCAG
CGCAATAGTGTGGATGCTATACTGGATACACTACCTTCCTGAAGTGCAGC
AGAAGTTGCGTGCCGAGCTGGATGCGTTGGGACCGGACCCTGATCCTATG
GCTATAGCACAGCTTCCCTACCTGACCGCCGTGTGCCAAGAAGCACTGAG
GATATATCCTATCACTCCAACCACATTTATTAGACGTTTAAGGGAGCCTA
TGACTCTTGCAGGTTACAGATTTAAAGCGGGTACGGCACTTATGCCGGCG
ACGTACATCATCCATCAAAGACCCGATCTATACCCCGAGCCTAAACAATT
TAGGCCAGAGAGGTTCCTAGAAAGACAGTTTGCTCCGCATGAGTTTTTAC
CATTCGGCGGGGGCCATAGATATTGCATCGGAAGTGCCTTGGCCATGATG
GAGTTGAAGTTATCAATTGCAACGCTGCTTGCGGATTTCGAACTGGCATT
ACTGCACTCCAGGCCGTTGCTGCCAGCTAGGAGAGGACTAACAATGGCTC
CACCAGCAGCAATGAAGTTAAGGATCAAGGCGAGAAAAACCAACAAAGCT

Seq. ID No: 214

>T5H_22ATGCCCGCGCCTAAAACTGCACCGAGCACACTGCCATTACCAC
CAGGAAGGTTAGGATTGCCGTGGATTGGTGAAACACTGTCATTTTTGAGG
GACCCAAACTTTGCGACAAAAAGACAGGCACAGTATGGTAGTTTGTTCAA
ATCTAGGATCATTGGCCAGCCGACCGTATTCTTTTGCGGACCAGAGGCGA
ATGCTTTCCTTCTGTCCAGCCACGCGGACTGCTTCTCCTGGAGAGATGGC
TGGCCAGGCACATTTCAAGAACTTCTTGGTGAGTCCTTATTTTTACAAGA
GGGTGAGACTCATTTACGTAACCGTAGACTGCTGATGCCAGCCTTTCACG
GAAAAGCGTTGGCAAGTTATTTCAGCACTATGGTTGCCCTTTCCGATAGC
TATCTGGCCCGTTGGGAGAAGAAACAGCAATTGACATGGTTCCTAGAGTT
CAAAAAGTTTACGTTTGAAGTAGCCTCTGTTCTATTGGTCGGCTCAGCCC
CAGGTCACGACGAGACCGACAACACTATCGGCACGGCTGAAAGTGCGGAG
ACGGAGGCCCAAATCGCGCAGCTGGCTTCCTGGTTTGCAGATCTAACGAA
CGGCTTATTCACCCTTCCTATCCGTTGGGGTCCCACGACCTATAGGAAGG
CCCTAAGAGGCAGGGATAGATTGCTGAGTTATATAGAACAGGAGATTACT
AAAAGGAGACAATTATTGGCACGTTTACAAACCGATCCAACCGCAGCTCT
ACCGACGGATGTTTTAACCTTACTTCTTCAAACGGAAGATGATGAGGGAA
ACAGACTTTCTGAGGCAGAAATCAAGGTTCAGACATTGCTAATGCTGTTT
GCGGGGCACGAGACGACGACCTCTATGCTAACTTCATTAGTTATGTCTCT
TGCCCAAAACCCGGACGTCCTAGCCAAAGCACGTGCGGAGCAACAAGCGT
TCCCTGCAGAGTCAGCCCTAACGTTTGAACAGATACAACAAATGCCATAT
TTAGATCAGATTCTAAAAGAGGTCGAACGTCAGTATCCTCCCGTCGGGGG
AGGATTCAGGAGGGTCATCAAGCCGTTCAACTTTAATGGATACCATGTCC
CTGCAGGTTGGCTGGCATTATACAGGATTGATGCGGCGCACAAGGACGAA
AGATGTTATACGAACCCGTCAGACTTTGACCCTGATAGATTCAGCCCTGA
GCGTGCTGAACAAAAACGTTATGACTACTCACTTGTTGGGTTCGGTGGGG
GCCCACGTGTGTGCCTAGGCATGGCGTTTGCCAAGCTCGAAATGAAAATA
ATGGCTGCACAATTGCTTAGACGTTATCACTGGCAACTAGACGCTGATCA
AGATCTGACTATGAACCCTGTACCTAGTTTGAGGCCTGCGGACGGTTTGA
AGGTCCGTTTCAGTAAGCTAAGTTTCACAGCC

Seq. ID No: 215

>T5H_23ATGTTGGATATGCCCTCCGTGAAACCTTGGCTAACCTTCTCAG
ACTGGGCCTCCAAGTTCGGTGATATAAGTCATTTAGAAATATTTGGTCAA
CACATTGTGGTCCTTAACAGTGCTAAAACTGCTGTTGAAATGTTAGATAG
AAAATCTAGCATCTACTCCGATCGTCCGGTATTACCAATGGGTGGGGAAT
TAGTAGGCTGGAGAAATACCCTGGTCCTATTACCGTATGGAGATAACTTC
AGGGAATATAGAAGGAATTTTCACAGAGTGATTGGAAGCCGTGCGGCTAT
GAGCGTTTACCACGCGATTGAAGAGGAGGAAACTCATAAGTTTCTACAAA
GAGTGCTGACGAAACCGGCTGATTTGAGTGCTCATGTAAGGACAACAGCC
GGTGCAATTATTCTAAGGATTAGTCATGGGTATCATATTCAAGAAGACGG
GGACCCATTTGTCAGTTTGGCGGATACAGCTGTGGATCAATTCTCCCGTA
GTACGGCTACAGGAGCGTTCATGGTTGACCTGATCCCGGCCCTAGCTTAC
GTACCAGAATGGTTTCCTGGAGCTAGTTTCCAAAGAAAAGCGCGTGAGTG
GAGGGCCACATTACACGAGATGGTCAATCAGCCATATAAGTTCGTGCAAG
ACCAGATGGCTGCCGGCATAGCCCCTAAGAGCTTTACCAGTAACCTGCTG
GAGGGACGTACATTAACGGAAGAGGAGGAGCATATTATCAAATGGTCAGG
TGCGTCTCTGTACAGCGGGGGTGCCGACACTACGGTTAGTGCCATTTATG
GATTTTTCCTGGCCATGACGCTTTATCCCGAGGCGCAGAAGAAAGCCCAG
GCCGAGATCGACGCAGTAGTCGGGAGTGATCGTCTACCCACATTTGCAGA
CAGGGAATCTCTACCTTACGCTGAAGCATTGGTCAAAGAAGTTCTTAGAT
GGTGTCCCGTCGTCCCGATTGTAGTTCCGCACCGTGTTACCGCAGATGAT
ATACATAATGGATATTATATTCCCAAGGGTACCTTAGTACTAGCTAATGC
TTGGTATATGTTGAGGGACCCGTCAATATATCCCGATCCAATGAACTTTA
ACCCCGATAGGTTCCTTCCGTCAGGCGGGAAGGAGCCCCCAACAGACCCG
CGTGATATTTGTTTTGGTTTTGGACGTAGAATCTGCCCTGGAATGCACTT
GGCGGACGCTAGTGTTTGGTTATCCGCCGTAATGTCTCTGGCGGTGTTTA
ATGTCTCAAAGGTGGTCGAAAACGGTGTGGAGATCACCCCTGAAGTCGAT
CCTAGTTCCGGAACTATCAGTCACCCGAAGCCCTTCAAATGTTCTATTAA
GCCAAGATCTGCTAAAGCACTAGAGCTAATTCAGCAAACGCCACATTAC

Seq. ID No: 216

>T5H_24ATGCACTTACCTCCAGGTCCAAGGCCGCTTCCATTCCTTGGGA
ATTTGCTTCAGATGAACAGACGTGGATTGTTGAGGAGCTTTATGCAGTTG
CAAGAGAAGTACGGCGATGTATTTACAGTGCACCTTGGTCCTAGGCCAGT
CGTAATTCTATGCGGGACCGATACAATTAGAGAAGCCTTGGTGGATCAGG
CCGAAGCGTTCTCCGGAAGAGGTACCGTTGCTGTTCTGCATCCCGTCGTC
CAAGGCTACGGAGTCATTTTTGCAAATGGCGAGAGATGGAAAATCCTACG
TCGTTTTAGTCTAGTTACTATGAGAAATTTCGGAATGGGAAAGAGGAGCG
TCGAGGAAAGGATAAAAGAGGAGGCACAATGCTTGGTTGAAGAGCTAAAG
AAATACAAAGGCGCGCTACTAAACCCAACTTCCATCTTTCAGAGTATTGC
TGCAAACATAATCTGTTCCATAGTTTTTGGGGAAAGATTTGATTACAAAG
ACCATCAATTTTTAAGGTTACTGGACTTGATTTATCAGACATTTTCTCTT
ATGGGCTCATTGAGTAGTCAAGTATTCGAGCTATTTTCCGGTTTTCTGAA
GTATTTCCCTGGGGTGCACAAACAGATTAGTAAAAACTTGCAGGAGATTT
TAAATTATATAGATCATTCCGTTGAGAAGCACCGTGCCACATTGGACCCA
AATACTCCCAGGGACTTCATCGACACATACCTACTTCACATGGAAAAGGA
AAAATCTAACCATCACACTGAATTTCACCATCAAAACTTGGTAATTTCCG
TCCTGTCCCTGTTCTTTGCAGGCACCGAGACCACTTCCACGACTTTACGT
TACTCATTTTTAATCATGCTGAAATATCCACACGTCGCAGAAAAGGTTCA
AAAGGAGATCGACCAAGTGATATCTAGTCATAGGTTGCCGACGTTGGATG
ACAGGATTAAGATGCCCTATACCGACGCAGTCATCCATGAAATTCAGCGT
TTTGCAGACCTAGCGCCAATAGGGTTACCGCACAGGGTTACAAAAGATAC
AATGTTCCGTGGATATCTGCTACCTAAAAATACCGAGGTATATCCGATTT
TATCCTCCGCACTTCATGATCCGAGATACTTCGACCATCCTGACACTTTT
AACCCGGAACACTTCTTAGACGCTAATGGAACGTTGAAAAAGTCCGAGGC
ATTCCTGCCGTTCAGTACAGGAAAGAGAACTTGTCTGGGTGAGGGAATAG
CACGTAACGAGTTGTTCATTTTCTTTACAGCCCTACTTCAGAATTTTAGC
CTTGCCAGCCCTGTCGCACCCGAGGACATTGATCTTACTCCAATAAACTC
CGGGGCGGGTAAAATTCCAAGTCCATACCAAATCAATTTTCTAAGTCGTT
GTGTCGGA

Seq. ID No: 217

>T5H_25ATGTATCTAATACCCGACTTTAGTAAAGAGACCTGGATACTAC
TTATCATCTTACTTGCGCTGTTGGCTTATTATGGTATTTGGCCCTATAGG
CTTTTCAAGAAATATGGGATACCTGGCCCTAAACCTTTACCTTTCTTTGG
TACCTTTCTGGAGAACAGGAATGGAGTGTTTGAATTTGATATGGAATGCT
TCAAAAAGTTCGGTAAAGTATGGGGGTTTTACGACGGCCGTCAGCCCGTA
TTGGCCATTATGGACCCCGTTATTATCAAGGCCATTCTGGTTAAAGAATG
CTATACAGTGTTTACGAATCGTAGGAATTTCGGGCTAAACGGTCCTCTTA
ACTCAGCAGTGTCTATTGCAGCAGACGATCAGTGGAAACGTATTAGAACA
GTGCTATCACCGACTTTTACTTCCGGGAAGCTGAAACAGATGTTTCCAAT
CATTAAGCAATATGGCGACTTGCTAGTCAAAAATATACAGAAAAAGGTAG
ATAACAAGGAGTTCATAGATATGAAAAATATATTTGGTTCATACAGTATG
GATATCGTTTTAAGCACTAGCTTTTCAGTAAACGTAGACTCACTAAATAA
CCCTAACGACCCATTCGTCACCAACGGGAGGAATCTGTTTACTTTCTCTT
TCTTAAATCCCCTATTTCTGACGACGCTACTGTGTCCTTTCCTGATTCCA
ATACTGGACAAGCTGAATTTCTGCTTTCTGCCCATTTCAGTTCTAAACTT
CTTTCAGGATGCCATTACATCTATCAAGAAGAATAGACAAAAAGGCATTC
ACAAGGATAGAGTAGACTTCCTTCAGTTGATGGTTGACGCACAAGCCAAT
GATTCAAAAGGGGGCGCAGACCACGGCTACAAGGAATTAACGGATACTGA
GATCATGGCTCAGGGGTTAATTTTCATAATCGCGGGGTATGAGACTACGA
GCACAACTCTAATGTTTCTAGCGTACCACTTGGCGACCCACCCTGACGTG
CAGACCAAATTACAGGAAGAAATTGATATCATACTTCCCAACAAAGCGCC
ACCTACATATGAGGCACTTATGCAAATGGAGTATTTGGACATGGTATTGT
ACGAAAACCTGAGGTTGTATCCGGCAGCAGGTCGTATTGAGAGAGTTTGT
AAAGCAACGACAGAAATTAACGGAGTTACCATTCCGAAAGGTGTTGTGAC
AGTGATTCCAGCTTTCGTCTTGCATCGAGATCCCGAGTTATGGCCCGAAC
CGGACGAGTTCCGTCCCGAAAGATTTTCCAAGGAAAATAGAGAAACGCAA
GACCCATATACTTTTCTTCCATTCGGGGCGGGTCCAAGGAACTGTATAGG
GATGAGGTTTGCACTTATTAACATGAAGTCAGTAATTACATTGTTGCTTC
AGAACTTTTCCTTCCGTACTTGCAAGGACACGCCAATCCCGCTACAAATA
GATACTAGGGGATTCTTGAAAACAACTAAGCCTGTCATCCTTAACTTGGT
CCCCAGGGAAGCTCAGAAAACCGAGAAA

Seq. ID No: 218

>T5H_26ATGTATGATACGTTCCTTGAATGGATCGAAAAGTATGGACCTG
TAGTACGTGTCAACTCATCTCACTCAACCTTCGTCATTGTGATTTCCCCC
GAGGGAGTTAAGGAATTTCTTATGTCACCCAAGTATACCAAGGATAATTT
CTATGAACGTATCGAAACCCTGTTTGGCGCCAGATTCTTAGGTAAGGGAC
TTGTGACCGATAGGGACTACGATCACTGGCACAAACAACGTAGGATGATG
GACCCCGCGTTCAGCAGGACGTATTTAATAGGTTTAATGGGGACCTTTAA
TGAGACTGCCGAGGACCTTATGGACGTGCTAGGTGACAAAGCGGACGGTA
AGTGCCAGGTGGGGATGCATGACATGTTATCTCGTGTGACTCTTGATGTT
ATTGCGAAGGCCGCTTTCGGGATGGAGCTGAACAGTTTACATGACGATCA
AACACCTTTCACAAGAGCGATAAGTACCGTAATGAAAGGGATGGTAGAGA
CCAGAAACCCTCTAGCACGTTACATACCGGGGAAACAAGCGTTTATACGT
GAGGTGAAAGAAAGTATCAAACTGCTGAGGGAGACTGGGAGAGAATGTAT
CCTTCAAAGGAGGAAGGAGATTCAAGATGGAGAGGATATACCGATGGATA
TATTAACGCAAATCTTGAAAGGGGCCGAGATCGAGGACGGATGCAGTTTA
GAAGATTTGATCGATAACTTCGTTACGTTCTTTGTCGCGGGACAGGAAAC
GACAGCGAATCAATTGAGTTTCGCGGTTATGGAATTAGCCAGGAACCCAG
AGATTCTTACCAGAGTACAAACGGAAGTGGATGAGGTACTTGGATCTAAG
CGTGATATCGAATATGAAGATTTGGGTAAGCTGCAGTATTTAAGCCAGGT
TTTGAAAGAAACGCTTCGTTTATATCCGATTGCTCCAGGAACATCCAGAG
CCCTGGAGAAGGAGACCGTTATTGAAGGTGTAAGGGTTCCACCCGGCACT
ACACTTATGTTCAACAGCTATATAATGGGACGTATGGAGAAGTATTACCA
CGATCCTTTTATCTTCAATCCAGACAGGTTCCATCCGGATGCGCCCAAGC
CAAGCTGCGCTTATTTCCCATTTTCCCTTGGACCACGTTCCTGTATTGGG
CAAGTTTTTGCTAGGATGGAAGCAAAGGTCGTCATGGCCAAGCTATTACA
GCGTTTCGAGTTTGAGTTGGTTGAAGGACAGTCTTTTAGAATCATGGATA
CGGGTACCCTCAGGCCGATGGATGGGGTTATATGCAGACTTAGACCGCGT
GCAGAGAGAAAATCCAGGAAG

Seq. ID No: 219

>T5H_27ATGGCCGCTCGTCCAAAGCCGGCAACCCCACCCAGCCCTCCAG
CGTTACCGGTTATCGGTCATTTGCATCTTCTAACAGATATGCCACATCAC
ACGTTTGCTGACCTTTCCAACAGCCTTGGTCCACTGATTTACCTTAGATT
GGGACAGGTCCCGACCATAGTGATTCATTCCGCACACCTAGCAAAATTAG
TTCTTAGGACACACGATCACGCTTTTGCTAACCGTCCTCAATTGATCTCC
GCTCAATACCTGTCTTTTGGTTGTTCCGATGTGACTTTTTCAAGTTACGG
AGCGTACTGGAGGCAGGCTAGGAAAATATGCGTCACGGAGTTACTAAGTG
CTAAAAGGGTACATTCTTTCAGATTGGTTAGAAAAGAGGAGGTGGATAGA
CTGTTGGATGCAGTGCTAACGAGCAGTGGAAAGGAGGTAGACATGTCCCA
AATGCTGTTTTGTTTGGCGAATGATGTACTATGTAAGGTTGCGTTCGGAC
GTCGTTTTATGGCAGAAAAGGACGGGAAGGGGAAGAACCTTGGATCTGTA
CTAATGGAAACCCAAGCGTTATTTGCTGGATTCTGCTTAGGAGACTTCTT
TCCCAAGTGGGAATGGGTTAATAGCATGTCAGGTTATAGGAAGAGACTGT
TGAAGAATCTAAAGGACTTGAAGGAAGTCTGTGACGAGATAATTGAAGAG
CATTTAAAGAAGAAGAAAAAGAAAAACGGCACAGAAAACGCCGATGACGA
CGATGACTATAACGAGAAGGAGGACTTTGTAGATGTTTTGCTTAGAGTGC
AGAAAAGGGAAGACCTTGAAGTACCCATCACTGACGACAACTTGAAGGCG
CTGGTCCTAGACATGTTCGTCGCTGGCACTGACACCAGCAGTGCTACTTT
AGAGTGGGTGTTCACAGAGCTTGCGAGACACCCCCGTGTTATGAAGAAAG
CTCAAGAAGAGGTTCGTATGATTGCTTCCGGAAATGGTAAGGTCGATGAG
TCAGACCTACAGCATCTTCACTACATGAAGGCGGTAATAAAGGAGACGAT
GAGGCTACACCCGCCAGTGCCCCTTTTAGTCCCAAGGGAGTCAATGGAAA
AATGCGCCCTAGACGGGTATGAAATCCCTGCCAAGACACGTGTCCTGATT
AACACTTACGCTATCGGTCGTGACCCGAAATCATGGGAAAATCCCCTAGA
CTATGACCCAGAAAGGTTCATGGAGGACGACATTGACTTTAAGGATCAAG
ATTTCAGATTCCTTCCTTTTGGCGGAGGGCGTAGAGGCTGCCCTGGTTAT
TCCTTCGGACTTGCGACAATTGAGATAACACTAGCCCGTCTTCTATATCA
CTTCGATTGGGCTTTACCGCATGGAGTAGAGGCGGACGATGTGGACTTGA
GCGAGGTGTTCGGACTAGCAACTCGTAAAAAGACTGCTCTGGTTTTAGTG
CCGACGGCAAACAAGGATTTCCAGTTTAGAGGGCATGACTTC

Seq. ID No: 220

>T5H_28ATGGGGAAAAACAAAGTGCCGCCAGGACCGATCGGACTGCCGT
TCATCGGTAACCTGCATCAGTTCGATACTCTAGCACCACATATCTACTTT
TGGGAGCTATCCAAAAAGTACGGAAAAATCTTCTCATTTAAGTTGACGAG
TAACGTGCCTATAATAGTGGTTAGCAGTGCAAAATTGGCGAAAGAAGTGC
TGAAGACACAGGATCTAGTGTTTTGCAGCAGACCATCACTGGTCGGACAG
CAAAAGCTGTCATACAATGGGCATGACATAGGCTTCGCACCATATAACGA
TTATTGGAGGGAAATGAGGAAAATTTGCGTACTTCACCTATTCTCTCTGA
AGAAGGTGCAGCTATTTTCCCCCATTCGTGAGGATGAGGTTAGCCGTATG
ATCAAGAAAATCTATCAACAAGCAGTGAACAGTCAAGTCACTAACCTATC
CAACCTAATGATCAGCTTAAATTCTACTATAATATGTCGTGTAGCGTTCG
GAGTAAGATTCGATGAAGAAGCCCATGAGAGAAAGAGATTCAACTACATA
TTGGCAGAAGCACAGGCGATGTTTGCGGGTTTCTTCATGTCAGATTTCTT
CCCATCACTTAGCTGGATAGATAAGTTGACAGGGATGATCGATAGGCTAG
AAAAGAATTTTAAAGACCTTGACGAATTTTATGAGGAACTTATAGAACAG
CACTACAATCCTAACAGGCCGAAAAGTATGGAGGGAGACTTTATTGATAT
TCTGCTACAGCTCAAAAAGGACCAGCTAACTCCTATAGACTTGAGCCTGG
AGGATATTAAAGGAATCTTGATGAATGTACTATTGGCAGGCTCTGACACC
TCTAGTAGTGTCATAATTTGGGCGATGACTATATTAATCAAGAACCCCAA
GGCAATGAAGAAGGTGCAAGAGGAAATTCGTAACCTAATCGGGAATAAAG
GGATAGTCAATGAGGATGACATCCAAAACATGCACTATCTAAAGGCCGTC
ATCAAAGAAACTCTTAGACTATTTCCGCCAGCACCTCTACTTATTCCTCG
TGAGTCAATGAAGATCTCTACTCTGGAGGGGTACGAGTTTCAGCCAAGAA
CTATCGTATACGTCAATGCATGGGCGATCGCTAGGGACCCGGAAATATGG
GAAAACCCAGAAGAGTTCATGCCAGAACGTTTTCTGAATAGTAACATTGA
TTTTAAGGGACAAGACTACGAACTAATACCATTTGGCGCAGGGAGGAGAG
GATGTCCGGGGTTGGCTTTGGGCGTAGCGTCAGTGGAACTAGCTCTATCT
AATCTACTTTATGCGTTCGACTGGGAGTTACCCTATGGCTTGAAGAAGGA
GGACATAGACATTAATGGTAAACCGGGGATCACAGTTAACAAGAAGAACG
ATCTGTGTCTTATCCCCAAGAAGTACTTT

Seq. ID No: 221

>T5H_29ATGAAGTTAACGGGGAAGCGTTATAGATTACCACCAGGACCTG
CCGGCGCTCCCGTGGTTGGGAATTGGCTTCAAGTGGGTGACGACCTGAAT
CACAGAAACTTAATGTCTTTGGCGAAGCGTTTCGGTGATATTTTCCTGCT
GCGTATGGGAGTTAGGAACTTAGTGGTGGTGTCAACGCCCGAACTTGCAA
AGGAGGTTCTACATACCCAAGGGGTAGAATTTGGGTCTCGAACACGTAAT
GTTGTGTTTGACATTTTTACAGGCAAAGGACAAGACATGGTTTTCACTGT
ATATGGCGACCATTGGAGAAAGATGAGGAGGATAATGACGGTACCTTTCT
TCACAAATAAAGTAGTAGCACAGAACAGGGTTGGTTGGGAGGAAGAAGCG
AGGCTTGTAGTGGAGGATGTAAGAAAAGACCCCCGTGCAGCCGCCGAGGG
AGTTGTAATCCGTAGACGTTTACAATTAATGATGTACAATGACATGTTTA
GGATAATGTTCGACACTAGATTTGAATCAGAGCAGGACCCCTTGTTTAAT
AAGTTAAAAGCATTGAATGCGGAGCGTTCACGTCTTTCCCAATCTTTTGA
ATACAATTACGGAGACTTCATACCTGTCCTGAGACCTTTTCTGAGAGGGT
ACCTAAATCGTTGTCATGATTTGAAAACCCGTAGAATGAAGGTTTTCGAG
GATAACTTCGTACAGGAACGTAAGAAAGTAATGGCGCAAACCGGAGAGAT
CCGTTGCGCTATGGATCACATTCTTGAAGCGGAGAGGAAAGGCGAGATAA
ACCACGATAATGTTCTTTATATTGTTGAGAACATCAATGTGGCTGCAATA
GAAACGACACTATGGTCTATCGAGTGGGGCATTGCGGAGCTAGTTAACCA
TCCCGCGATTCAAAGTAAACTACGTGAAGAAATGGATAGTGTACTGGGCG
CCGGAGTACCCGTGACCGAGCCTGACTTAGAAAGACTACCCTATCTACAA
GCCATTGTTAAAGAAACACTTCGTCTTCGTATGGCGATACCTCTGTTAGT
CCCCCATATGAACCTAAATGATGGGAAACTAGCTGGATATGACATCCCCG
CGGAATCAAAGATATTAGTTAACGCCTGGTTCCTTGCAAATGACCCAAAG
AGATGGGTCAGACCTGACGAGTTCAGACCAGAGCGATTTTTAGAGGAGGA
AAAGACAGTAGAGGCGCATGGGAATGATTTTAGATTCGTACCATTCGGGG
TAGGAAGAAGGTCATGCCCAGGTATCATTTTAGCTTTACCAATCATCGGC
ATCACGCTAGGCAGATTGGTCCAAAACTTCCAGCTGTTGCCCCCGCCTGG
GCAGGATAAGATAGATACGACAGAAAAGCCGGGTCAGTTTTCTAACCAAA
TCGCCAAACATGCTACAATTGTGTGTAAGCCCCTAGAAGCG

Seq. ID No: 222

>T5H_30ATGAAACCGCGTGGCGCTAAATATCCTAATAGTCTTCCATGTT
TGCCCTTCATCGGCTCACTGTTGCACCTTGCATCACACCTGGCTCCTCAT
ATCTTATTTAATAAACTACAAGAAAAATATGGGTCACTATATTCATTCAA
AATGGGATCTCACTACATCGTAATAGTAAATCATCATGAACATGCTAAGG
AGGTATTACTGAAGAAGGGTAAAACATTTGGTGGTCGTCCTAGAGCTGTG
ACCACAGACTTGCTAACTAGGAATGCTAAGGATATTGCTTTTGCGGACTA
TAGCCCCACGTGGAAGTTCCATCGTAAACTTGTACACGCAGCACTGTCTA
TGTTTGGTGAGGGGACTGTAGCGATAGAGAAAATCATTTCACGTGAAGCC
GCCTCCCTTTGTCAAACTCTTATTACATTTCAAGGGAGCCCCTTAGATAT
GGCCCCGGAGCTGACCCGTGCGGTGACGAACGTAGTATGCGCACTTTGTT
TCAACGCGAGATACAAAAGATGTGATCCTGAATTTGAAGAGATGCTTGCA
TATTCCAAAGGAATCGTTGACACGGTCGCTAAAGATAGTCTGGTAGACAT
TTTTCCCTGGTTGCAAATATTTCCCAATAAAGACCTGGAGATTCTGAAGA
GATCAGTGGCCATTAGAGACAAACTATTACAGAAAAAGCTGAAGGAACAT
AAGGAGGCATTTTGTGGGGAAGAGGTGAATGACCTGCTGGACGCGTTATT
AAAAGCAAAGCTGTCAATGGAAAACAACAACTCAAATATATCCCAGGAAG
TCGGGCTTACAGACGACCATCTTTTGATGACCGTGGGTGACATATTTGGC
GCGGGAGTTGAGACAACCACCACAGTACTAAAGTGGGCTGTAGCGTATCT
ACTACACTATCCGAAGGTCCAAGCTAAGATTCAAGAAGAATTAGATGTCA
AGGTAGGTTTCGGGAGACATCCGGTTTTGTCCGATCGTCGTATCCTGCCC
TACTTGGACGCGACGATTAGCGAAGTCCTAAGGATAAGACCAGTTGCCCC
TCTACTGATCCCCCACGTCGCATTACACGAAAGCAGTATAGGGGAATATA
CCATCCCACAGGATGCGAGAGTTGTAATAAATCTGTGGAGCCTGCATCAC
GATCCTAACGAATGGGAGAACCCCGAGGAGTTCATTCCTGACCGTTTTCT
TGACGAAAATGGGAACCATCTTTACACTCCTAGCCAAAGCTACCTACCCT
TCGGTGCAGGCATTCGTGTTTGTTTGGGGGAGGCATTGGCTAAGATGGAG
ATTTTTCTATTTCTGTCATGGATCTTACAGAGGTTCACGCTAGAAGTCCC
TGCCGGGGATTCCCTGCCGGATCTTGACGGGAAGTTTGGCGTGGTGTTGC
AAGTGAAGAAATTTCGTGTAACAGCGAAGCTGCGTGAAGTGTGGAAGAAC
ATCGACCTTACAACA

Seq. ID No: 223

>TAT2ATGACGGAGGATTTCATTTCTTCTGTTAAAAGATCCAACGAAGAA
TTAAAAGAGAGAAAATCCAATTTTGGATTTGTGGAGTACAAATCAAAACA
GCTGACGAGCAGTAGCTCACACAATAGCAATAGCAGCCATCACGATGATG
ACAACCAACACGGGAAAAGGAATATATTTCAAAGGTGTGTTGACAGTTTC
AAAAGTCCCTTAGACGGATCTTTTGACACATCAAATCTAAAACGTACGCT
AAAGCCCAGACATCTTATCATGATCGCTATAGGTGGGAGTATAGGAACTG
GTCTGTTCGTCGGGTCAGGTAAAGCCATTGCCGAAGGTGGTCCATTAGGG
GTGGTCATTGGCTGGGCCATCGCTGGCAGTCAGATCATTGGTACCATCCA
CGGACTAGGCGAAATAACCGTCAGATTCCCCGTGGTCGGCGCGTTCGCGA
ATTACGGAACTAGGTTCTTAGATCCATCAATATCATTTGTCGTGTCTACT
ATCTACGTACTTCAATGGTTCTTCGTACTACCGCTGGAGATCATCGCAGC
GGCTATGACAGTGCAGTACTGGAACTCATCAATTGATCCGGTCATCTGGG
TTGCGATCTTCTATGCCGTCATAGTCAGCATAAATTTGTTTGGTGTACGT
GGATTTGGAGAGGCAGAATTTGCATTCTCTACGATAAAGGCCATCACGGT
GTGCGGTTTCATAATACTATGCGTGGTACTTATTTGCGGCGGGGGTCCTG
ATCACGAATTTATAGGGGCCAAGTACTGGCACGATCCAGGCTGTCTAGCA
AATGGGTTTCCGGGCGTACTTTCCGTACTGGTCGTTGCCTCATATAGCTT
GGGCGGTATAGAGATGACCTGCTTAGCGTCAGGGGAGACGGACCCCAAGG
GATTACCGTCTGCCATAAAGCAGGTGTTCTGGAGAATACTGTTCTTCTTT
CTGATTTCTCTTACGCTTGTTGGATTTCTTGTCCCGTACACGAATCAGAA
TTTACTGGGAGGGTCCAGCGTCGATAACAGCCCCTTCGTGATCGCAATAA
AGCTGCACCACATCAAAGCCCTTCCCTCAATCGTCAACGCGGTAATCTTG
ATCAGTGTTCTGAGTGTAGGTAACAGTTGTATCTTTGCGTCCAGTAGAAC
ATTGTGTTCAATGGCCCACCAAGGCTTGATTCCCTGGTGGTTCGGTTACA
TAGATAGAGCAGGCAGACCTCTAGTGGGAATTATGGCAAACAGTTTGTTT
GGACTTCTAGCTTTCTTAGTGAAGTCAGGCTCAATGAGCGAAGTATTCAA
CTGGTTAATGGCGATAGCGGGGCTGGCCACTTGCATAGTCTGGTTATCTA
TAAATCTTTCCCATATACGTTTTAGGTTGGCAATGAAAGCGCAAGGCAAA
TCCCTAGATGAGCTAGAGTTTGTAAGTGCAGTTGGTATATGGGGTTCCGC
TTATTCTGCGTTGATAAACTGCTTGATATTGATTGCGCAATTCTATTGTT
CTCTGTGGCCGATAGGTGGATGGACGTCAGGAAAAGAAAGAGCCAAAATC
TTTTTCCAAAACTATCTGTGCGCCCTAATTATGCTGTTCATTTTCATTGT
TCATAAAATCTATTACAAATGCCAAACTGGTAAGTGGTGGGGTGTCAAGG
CTTTGAAGGACATTGACCTGGAAACAGACAGAAAAGACATTGATATAGAA
ATTGTGAAGCAAGAGATCGCCGAAAAGAAAATGTACCTAGATAGCAGACC
CTGGTATGTAAGACAGTTTCATTTCTGGTGC

Seq. ID No: 224

>TMO_1ATGAGCACGTTAGCAGATCAGGCATTGCATAACAACAATGTCGG
CCCGATTATTCGTGCAGGGGACCTAGTCGAACCTGTCATCGAAACTGCCG
AAATCGACAATCCCGGGAAGGAAATAACGGTCGAAGACCGTCGTGCCTAC
GTAAGGATTGCGGCAGAAGGGGAGCTAATATTGACTCGTAAGACTCTTGA
GGAACAGCTAGGTAGACCGTTTAATATGCAGGAACTAGAGATAAATCTAG
CCTCATTCGCTGGGCAAATACAGGCAGATGAAGACCAAATCAGATTTTAT
TTCGATAAGACAATGGGCGGAGGGAGTGGTGAGGGAAGAGGCAGCCTTCT
TACTTGTGGAGACGTGGAAGAAAACCCCGGGCCCATGTTTAACATACAAT
CTGACGATCTATTACATCACTTTGAAGCTGATAGTAATGATACACTATTG
TCCGCCGCGCTTCGTGCTGAACTTGTATTCCCCTACGAATGCAACTCAGG
TGGTTGCGGCGCTTGTAAAATCGAACTACTTGAAGGAGAAGTATCCAATT
TATGGCCGGACGCACCTGGGTTAGCCGCCAGAGAGCTACGTAAGAATAGG
TTTCTGGCCTGCCAGTGTAAGCCGCTTTCTGATCTTAAAATTAAAGTTAT
CAATAGAGCCGAGGGCCGTGCTTCACATCCCCCGAAGAGGTTCTCTACGA
GAGTTGTAAGCAAAAGGTTTTTAAGTGACGAAATGTTTGAACTACGTCTG
GAGGCCGAACAGAAGGTCGTTTTCTCCCCCGGACAGTATTTTATGGTGGA
CGTTCCAGAGTTAGGCACCAGGGCATATAGCGCCGCGAACCCAGTTGATG
GTAATACACTAACGCTAATCGTAAAAGCCGTGCCGAACGGAAAGGTGAGC
TGCGCTCTAGCTAATGAGACGATCGAGACACTACAACTTGACGGACCTTA
TGGTTTGAGCGTGCTGAAGACTGCTGATGAAACGCAGTCCGTATTTATCG
CGGGAGGGAGTGGTATAGCTCCTATGGTCTCTATGGTCAATACTTTAATA
GCACAAGGCTACGAGAAGCCTATTACGGTCTTCTACGGTTCTCGTTTGGA
GGCGGAACTAGAGGCAGCCGAGACATTGTTCGGCTGGAAGGAGAACCTAA
AATTGATTAACGTGAGCAGTTCCGTCGTTGGTAATTCCGAGAAAAAGTAT
CCCACCGGGTACGTGCATGAGATCATACCGGAATACATGGAAGGCCTGTT
AGGTGCGGAGTTCTACCTGTGTGGTCCACCACAAATGATCAACTCTGTCC
AGAAACTGTTGATGATTGAAAATAAGGTCCCATTCGAGGCGATTCATTTT
GATAGGTTCTTC

Seq. ID No: 225

>TMO_2ATGGCCATGCATCCGCGTAAAGATTGGTATGAGCTGACGCGTGC
GACCAATTGGACACCTTCATACGTGACGGAAGAACAACTGTTTCCAGAAC
GTATGAGCGGTCACATGGGTATTCCACTTGAGAAATGGGAGTCCTATGAC
GAGCCGTACAAGACTTCGTACCCGGAGTATGTTTCCATACAAAGAGAGAA
GGACGCGGGTGCCTATTCTGTTAAAGCTGCCCTGGAACGTGCGAAGATTT
ATGAAAATAGTGATCCCGGTTGGATTAGTACATTGAAGTCCCACTATGGC
GCTATTGCCGTGGGTGAATACGCAGCGGTTACTGGTGAGGGTAGAATGGC
TAGATTCAGTAAAGCCCCTGGCAATAGGAACATGGCTACTTTCGGTATGA
TGGACGAGCTGAGGCATGGTCAATTACAACTATTTTTCCCCCACGAATAC
TGCAAAAAGGACAGGCAATTCGACTGGGCCTGGAGAGCTTACCATTCTAA
CGAATGGGCCGCCATCGCGGCCAAACACTTTTTCGACGATATCATCACAG
GCAGGGACGCTATATCTGTCGCGATCATGCTGACATTTTCATTCGAAACG
GGTTTCACAAACATGCAGTTTCTAGGACTTGCTGCTGATGCAGCCGAGGC
GGGAGACTACACGTTTGCGAACCTTATATCAAGCATTCAAACCGATGAAT
CCAGACATGCACAGCAGGGTGGTCCCGCATTACAACTGCTAATTGAAAAC
GGTAAGCGTGAGGAAGCGCAGAAGAAAGTCGATATGGCAATTTGGAGGGC
CTGGAGATTATTCGCAGTGCTGACCGGTCCGGTAATGGACTATTATACGC
CCTTGGAGGATCGTTCCCAAAGTTTCAAGGAGTTCATGTATGAATGGATC
ATTGGCCAGTTCGAACGTAGCCTGATTGACTTGGGTTTGGATAAACCCTG
GTACTGGGATTTGTTCTTAAAGGACATCGACGAGCTACACCACAGTTATC
ACATGGGAGTTTGGTACTGGCGTACCACCGCCTGGTGGAACCCTGCTGCA
GGGGTAACTCCGGAGGAGAGGGATTGGTTGGAAGAAAAATATCCGGGATG
GAATAAGCGTTGGGGCAGGTGCTGGGACGTGATAACCGAGAATGTCCTAA
ATGACAGAATGGATTTAGTGTCTCCAGAGACATTACCCTCTGTCTGCAAC
ATGTCTCAGATTCCATTAGTTGGAGTACCGGGCGACGACTGGAACATAGA
GGTCTTTAGTCTGGAACATAACGGCAGATTGTATCACTTCGGAAGTGAGG
TAGACAGATGGGTTTTCCAGCAAGATCCGGTCCAATATCAAAATCATATG
AACATCGTTGATAGATTCCTGGCGGGCCAAATTCAACCCATGACATTAGA
AGGTGCGCTTAAATACATGGGGTTCCAGTCCATCGAGGAAATGGGGAAGG
ACGCTCACGATTTCGCATGGGCAGATAAGTGCAAACCGGCAATGAAGAAG
TCAGCAGGTGGGGGCAGCGGAGAAGGCAGGGGAAGCCTGTTAACCTGTGG
CGACGTAGAGGAGAATCCCGGTCCTATGTCATTCGAAAAGATTTGCTCTC
TGGATGATATCTGGGTTGGTGAAATGGAGACGTTCGAAACTTCAGATGGA
ACAGAGGTTTTGATCGTGAACTCTGAGGAACACGGCGTTAAAGCATACCA
GGCTATGTGTCCTCACCAGGAGATTCTTCTAAGTGAGGGGAGTTACGAGG
GAGGTGTCATAACGTGTAGAGCGCACTTATGGACATTCAATGACGGCACG
GGCCACGGCATTAACCCCGATGACTGCTGTTTAGCGGAGTACCCTGTGGA
GGTTAAGGGTGATGACATTTATGTCAGTACCAAAGGTATACTTCCCAATA
AGGCACACAGC

Seq. ID No: 226

>TMO_3ATGTCTTTTACCAAGGTTTGTTCTGTCGGAGATATCTGGGAGGG
GGAAATGGAGCCATTTACTGTTGATGGTCACGAGATATTACTTGTAGGTG
TGGAAGGGGGAGGGATCAAAGCCTTCCAGGGTATCTGTCCTCACCAAGAT
ATAGCGTTGTCAGAGGGGAAATTTGATGGGAAGAAACTTATCTGCCGTGC
ACACCTATGGCAATTCGATGCCAGCAACGGAAAAGGTATCAACCCAGACG
ATTGCGCCCTAGCCGAATACCCTGTCAAGGTTGACGGCGACGACGTCTAC
GTACAGACTGCTGGGGTGGAAGCATTATTTGCACATAGCGGTGGTGGCTC
AGGCGAAGGGAGGGGCTCCCTGCTAACGTGCGGAGATGTCGAGGAAAACC
CTGGTCCGATGGCCTTGCTGAATAGAATGGACTGGTATGACCTTGCGCGT
ACAACTAACTGGAGTCCAAAATATGTAACGGAGTCTGAATTGTTTCCGCC
CGAGCTGTCAGGAGACCACGGAATACCTATGGAGAAGTGGGAAACTTATG
ATGAACCCTACAAGCAGACTTATCCAGAGTATGTTAAAGTACAAAGGGAA
AAGGATGCGGGCGCCTATAGTGTTAAGGCAGCCCTGGAAAGATCCCAGAT
CTATGAGCGTTCTGACCCGGGGTGGCTAACGGTTATGAAGCAGCACTATG
GCGCGATTGCACTTGGAGAGTACGCCGCTTCATCAGCAGAGGCCAGGATG
ATGAGATTTAGTAAAGCTCCAGGTATGAGAAACATGGCCACACTAGGGAG
CATGGATGAAATACGTCACGGACAGATACAGCTATATTTTCCCCATGAGC
ATGTTTCCAAGGATCGTCAGTTTGATTGGGCAGCTAAGGCTTTCCACACC
AACGAATGGGCAGCGATAGCTGCGCGTCACTTTTTCGACGATATAATGAT
GACCAGAGATGCTATAAGCGTAGCAATTATGCTTACATTCTCATTCGAGA
CGGGGTTTACGAATATGCAGTTTCTTGGCTTGGCAGCAGACGCGGCGGAG
GCTGGAGATCACACATTTGCAAGTTTGATATCCTCCGTACAAACAGATGA
GAGCAGGCATGCCCAGATTGGAGGCCCTACGCTGCAAATCCTTATTGAGA
ACGGCAAGAAGGCTGAAGCCCAAAAGAAGGTGGATATCGCTTTCTGGCGT
GCCTGGAGACTGTTTTCCGTCCTGACTGGTCCAGTCATGGATTACTATAC
GCCGCTAGAACACCGTAAACAGAGCTTTAAGGAATTTATGCAAGAATGGA
TAGTCGCGCAGTTCGAGAGAGCACTTAGCGATCTAGGGCTGGATAAACCC
TGGTATTGGGATACGTTCTTACAGCAGCTTGACCAACAGCATCACGGGAT
GCACCTGGGGGTCTGGTATTGGAGACCAACGGTTTGGTGGAACCCTGCTG
CTGGCGTTACACCAGCAGAAAGGGATTGGCTGGAAGAAAAATACCCCGGC
TGGAACGATACTTGGGGACAATGCTGGGACGTGATTATTGACAATTTAGT
TGACGGCAACATAGCTCAAACATATCCGGAGACATTGCCGATTGTCTGCA
ACATGTGCAATCTGCCTATAAACTGCACGCCCGGCAATGGTTGGGCCGTG
CAAGATTACCCGCTGGAATACAACGGGCGTCTATACCACTTTGGGAGTGA
GCCTGATCGTTGGTGTTTTGAACAGGAACCTGAGCGTTATGCGGGCCACA
TGACCCTTGTCGATCGTTTTCTGGCTGGTCTAGTGCAACCAATGGACCTG
GGCGGGGCCCTGGCCTATATGGGGCTGGCGCCAGGGGAAATAGGGGACGA
TGCACACGGGTATAGCTGGGTCGATATTTATAAGAAAATGCGTATGAAGA
AGGCAAGC

Seq. ID No: 227

>TMO_4ATGTCAGTTGCATCAAGCGCGCAAGCGTATCATAACAACATGGT
CGGACCCGTTATGAGAGCAGGCGACTTAGCGTTAGCAGTAATTGAAGCGG
CCAGGGTAGACAATCCAGGCAAGGAGGTTTTTGTTGATGATAAACGTGCT
TATGTACGTATTCACACCGAGCAAGAAATGATATTGAGGAGAGAAACGAT
AGAGGAGGAACTTGGTAGACCATTTAAGATGAATGATCTTGAGGTAGATT
TAAGTAGTTTTGCGGGGCAAATCGAATCTTTGGATGACGCCGTTAGATTT
TACTTCACAAAGAAGTTGGGTGGGGGATCTGGCGAAGGGAGGGGTAGCCT
ATTGACATGTGGGGACGTGGAGGAAAATCCGGGACCATCATCAAACCCAA
TAATCCATAATCAAAAGGACGGCTCAAGATTCGCACAAAGGGAGGGGGAT
ACAATACTTCGTGCAGCTCTGAGGGCTGGTGTTGGGCTGTCCTACGAGTG
TAACTCCGGTGGCTGTGGAGGGTGCAAGTTCGAACTACTGGAGGGAGAAG
TGGACACGTTGTGGCCGGATGCTCCCGGGCTATCCGACAAAGACAGAAGG
AGAGGGCGTCACCTAGCTTGCCAGTGTCGTGCTAGGGGGCCTGTCAGTAT
AAAAGCCGCGACGGGAGCCGAGTACGTGCCTAAGGTCGTTCCACAAAGAC
AAACGGCTAGACTGGTTGGGAGCACTGATATTACGCACGACCTTAGAGAG
TTTAGGTTCAGATCCGCGGCAGGAGCCAGTTTCTTGCCCGGGCAGTTCGC
CATGCTAGATCTACCCGGGCTTGCGAGTGCTAGGGCCTATTCTATGAGTA
ACACTGCCAATGATGATGGGGAGTGGCATTTCCAAGTGAGGAGAGTCCCG
CATGGGCAAGGGACTCATGTATTGTTTGACAGGCTGGGGGTCGGAGACGA
AATCGGGTTGGACGGACCTTACGGAGTAGCCTGGCTAAGAACTGGTGCTC
CCAGAGATATAGTATGCGTAGCGGGTGGGAGTGGACTGGCTCCTATGGTC
TCCATCGCAAGAGGAGCCGCCGCCGCCGGCATGCTAAAGGATAGGAAACT
GTATTTCTTTTATGGCGCAAGAACTCCTAGGGACGTTTGTGGAGCGGAGA
TGCTTGCACAGCTGGATGGCTTCGGGGAACGTATCATATATCTTCCGGTG
GTTTCTCTTCCGGGTGGCGAGGGTGAATGGCAGGGGGAGACTGGTTACGT
ACACGATGCAGTAGCTAGGACACTACCCGGCTCATTGGCGGGTTTCGAGT
TTTATTTTGCTGGGCCGCCGCCGATGACGCAAGCCCTGCAAGAGATGCTG
ATGGTAGGACACAGGGTTCCTTTTGAGCAAATTCATTTTGATCGGTTTTT
C

Seq. ID No: 228

>TPH_1ATGCCGTCCAGACTGAATAAGGATGAATATCAGTTTTACATCGA
TCTTGATAATAAGTCCACACCAGCCTTGAATGAAATTGTTAAATGTTTAC
GTCTTGACATTGGCGCGACAGTGCATGAGTTGAGCAGAGATAAGAAGAAG
GATGCGGTGCCGTGGTTTCCGAAAACAATACAAGACTTGGACAAATTCGC
GAACCAGATCCTTTCCTACGGCGCTGAACTTGATTCTGATCACCCTGGCT
TCACTGATCCCGTCTATAGAGCTAGAAGGAAAGAATTTGCTGACATTGCT
TTTCATTATAAGCACGGCCAGCCAATCCCTTGCGTAACGTACACAGAGGA
AGAGAAGAAGACTTGGGGCACAGTATTCAAAGAGTTAAAGTTGCTTTATC
CGACTCACGCATGCTACGAACATAATCACGTTTTTCCTTTACTAGAGAAA
TATTGCGGTTATAACGAGAACAACATTCCTCAATTAGAGGACGTCTCTAA
ATTTCTTCAGACCTGTACGGGATTTCGTCTGAGGCCGGTTGCAGGCCTTC
TGAGTTCCAGGGACTTCTTAGCTGGCCTAGCTTTCCGTGTATTCCACTCA
ACTCAATACATTCGTCACTGGAGTAAGCCAATGTATACGCCTGAACCGGA
CATCTGTCACGAGCTATTGGGCCACGCTCCGCTTTTTGCAGATCCAAGTT
TCGCTCAATTCTCACAAGAAATCGGCCTGGCGTCACTGGGCGCTCCAGAT
GAATACATAGAGCGTCTAGCGACTCTTTACTGGTTCACAGTAGAGTTCGG
ATTATGTAAGCAGGATGACAAAATTAAGGCCTATGGAGCCGGACTTCTTT
CATCTTTCGGCGAGCTTCAGTATTGCCTTACAGATAAGCCTGAATTGAAG
CCCTTTGAGCCAGAAAAGACGTCCCTTCAGAAGTACCCGATTACAGAATT
TCAACCTGTGTACTTCATAGCCGAGTCATTCGAAGACGCTAAAGAGAAAA
TGAGAAAATTCGCCACTACTATTCCTAGACCGTTCAGTGTGCGTTATAAC
CCGTATACTCAAAGTATCGAGGTCTTAGACAATGTCCAGCAGTTAAAGAA
TTTAGCGGATTGTATAAACTCAGAGATCGGAACCCTGTGTTGCGCG

Seq. ID No: 229

>TPH_2ATGATCGAGGATAATAAAGAAAATAAAGATCACAGTCTGGAACG
TGGCAGGGCTTCATTGATATTCTCTTTAAAGAATGAGGTAGGCGGCTTGA
TAAAGGCGCTTAAAATATTCCAAGAGAAACATGTTAACCTATTGCATATC
GAGAGTAGAAAAAGTAAAAGGCGTAACTCCGAGTTCGAGATATTCGTAGA
TTGTGATATTAACAGGGAACAGCTGAATGACATTTTCCATCTTCTAAAGA
GCCACACTAACGTACTGTCTGTAAACCTACCGGACAATTTTACCCTAAAG
GAAGATGGTATGGAAACAGTCCCTTGGTTTCCCAAAAAGATAAGTGATTT
GGACCACTGCGCTAACAGAGTGCTAATGTATGGAAGCGAGTTAGATGCCG
ACCATCCTGGATTTAAGGACAACGTTTATAGGAAGAGGAGGAAGTATTTC
GCTGATTTGGCGATGAATTATAAACATGGCGATCCAATTCCCAAGGTTGA
GTTCACTGAAGAGGAGATTAAAACTTGGGGAACCGTTTTTCAAGAATTAA
ATAAGTTGTATCCGACACACGCATGTAGAGAGTATCTAAAGAACCTTCCT
CTTCTGAGTAAGTATTGTGGGTATAGGGAGGACAATATCCCGCAACTGGA
GGATGTTAGCAACTTCTTGAAAGAAAGGACCGGATTCAGTATCCGTCCAG
TGGCAGGCTATCTATCACCTAGAGATTTTCTAAGTGGGTTAGCATTCAGG
GTATTCCATTGCACACAATACGTCCGTCACAGTAGTGATCCGTTCTATAC
ACCAGAGCCCGATACATGCCACGAGCTTTTAGGCCACGTTCCTTTACTTG
CGGAGCCGTCCTTTGCACAATTCTCTCAGGAGATCGGACTTGCATCCCTT
GGCGCTTCCGAAGAAGCAGTTCAGAAACTTGCCACTTGCTACTTCTTTAC
TGTAGAGTTTGGCTTGTGCAAGCAGGATGGGCAACTGAGAGTGTTTGGGG
CGGGATTGCTTTCCAGCATCAGCGAGTTGAAGCATGCGTTAAGCGGACAT
GCAAAAGTCAAGCCATTCGATCCAAAAATCACATGCAAGCAGGAGTGCTT
GATCACAACTTTCCAGGACGTTTATTTCGTGTCTGAATCTTTTGAAGACG
CCAAGGAGAAGATGAGGGAGTTCACGAAAACTATTAAGCGTCCTTTCGGG
GTAAAATATAATCCGTACACAAGATCCATTCAGATTCTGAAAGACACAAA
ATCAATCACGTCTGCGATGAACGAACTTCAACACGACTTGGATGTAGTTT
CAGATGCCCTAGCTAAGGTTTCCAGGAAGCCTTCTATA

Seq. ID No: 230

>TPH_3ATGTCCGGTCTAGCACTTGACCGTAGTTCACAGCCACACGAGGT
GAGAACACTGGAGGTCAACGAGTTGGACCCGAAAGTTTTCGCGGTAGTCG
AAGTAAGGAAGGACGAGCCAGGAGTACTGGGCGATGTGTTGAAAGTATTT
ACAGAGTCTAGCATAAACATCACTAACATTGAGTCCAGGTTCAAGAGCTT
CGCAAGGGATGGGCCAGCATTTCATATTGATTTTGAAGGTGAAGCCAGGG
AGCACAGAGTACAACGTGTGCTAAGGGATGTAAAATCTGTTCCAGGCGTC
TCTCAGGTGACAGTAATGGAAGAGAGGGAAGTCCCCTGGTTCCCAATTAA
TATTAGGGACTTAGATCTAACCACGGATACTCTTGACGGAGGAACAGCCT
TGATCAATGAGGATCATCCTGGCTTCAACGACCTAGCGTACAGGCAGAGA
AGAGAGGAGATCGTTACCGCTGCAAAGGAACATAGGCACGGAGACAGAAT
TGCCAGGGTACAGTACTTGGAACATGAAGTCGAGACTTGGAGGGCAGTCT
ACGAACAACTTCGTGAATGTCATTCAAGGTGGGCTTGTACTGAGTACCTA
GAGATGTTGCCGCAAATGGAGAGGTTCTGCGGTTATGCACCCGGGAACAT
ACCGCAACTAGCAGATATATCTGACTTTTTACAACAGAGAACTGGGTTTA
CCTTGCGTCCTATTACAGGTCTATTATCTGCTAGAGATTTTCTGAACGCA
CTTGCATTCAGAGTTTTCTACAGCACCCAATACATCAGGCACCACGGAAA
TCCCTTTTACACACCAGAACCGGATATTTGTCATGAGCTGATGGGACATG
TACCTCTTTTTGCGAATGCCGCTTTTGCCGATTTTAGTCAGGAAATAGGG
TTGGCATCCTTGGCTGCCAGCGACGATGATATAGCCCGTCTTGCGGCAGT
ATATTGGTTCACAGTTGAATTTGGGCTGGTCAGACAGGGAGGAGAGGTGA
AAGCGTACGGGGCTGGACTGCTGTCATCCTTTGGTGAGATGGAGTGGTCT
TGTTCCAGAGAACCCAGCACGACCTGTAGAGAAATGGGTAGCGTAGCGGA
GCTGCAAGCTCCTTCCATAGTCCCACTGGACCCGACCCAAGCAGGCAAAC
AGGCTTACCCTATAACCACGTATCAACCCTTGTACTTTTGTGCCGAGTCA
ATGCAGGACGCTAAGGCCAAGATCTCCCAGTTTTGCGATACTCTGACAAG
GCCCTTCTTCCCTCAATATGATCCGTTAACGCAAAACATCCGTGTTACTA
AAGCAGTACGTCGTGCGAGGCGTATCTCTACTGTAGAAATGCAGATGGCA
AAGCAACTTGATTATTTCGAGAAGCAA

Seq. ID No: 231

>TPH_4ATGGCAGTTCCCTGGTTTCCGAAAACTATACAAGACTTAGACAA
GTTCGCCAATCAAATCCTAAGTTATGGTGCCGAATTAGATTCAGACCACC
CTGGGTTCACGGACCCTGTATACAGAACGCGTAGAAAGGAGTTTGCTGAC
ATAGCGTTTCATTATAAGCATGGCCAACCAATTCCACGTGTAACGTATAC
AGAGGAGGAGAAGAAGACCTGGGGTACGGTGTTCAAGGAGTTAAAATTGC
TATACCCTACACACGCGTGTTATGAACACAATCATGTATTTCCACTTCTT
GAAAAGTACTGCGGGTACAATGAAAACAACATACCCCAGCTTGAGGACGT
TTCAAACTTCTTGCAGACGTGTACTGGGTTTAGGCTTAGGCCCGTGGCGG
GTTTACTAAGTTCCAGGGATTTTCTGGCTGGACTGGCGTTTCGTGTATTT
CACAGCACACAATATATCAGACATTGGTCTAAACCGATGTATACGCCGGA
GCCGGATATTTGTCACGAGCTTCTAGGTCATGCACCTTTATTTGCTGACC
CTAGCTTCGCACAGTTCAGTCAAGAAATTGGCCTGGCTTCACTAGGCGCT
CCAGATGAGTATATTGAGAGACTTGCCACATTATACTGGTTTACCATTGA
GTTTGGCTTGTGTAAACAAGACGACAAGATAAAAGCCTATGGGGCGGGGT
TATTATCTAGTTTCGGTGAGCTACAGTATTGTCTAACGGACAAACCGGAC
TTAAAGCCGTTTGAGCCAGAAAAGACATCATTACAGAAATATCCCATAAC
CGAGTTCCAGCCTGTCTATTTCATAGCGGAGAGCTTTGAAGATGCCAAAG
AGAAAGTTCGTAAATTCGCAACGACGATTCCGAGGCCCTTCAGCGTACGT
TATAATCCTTATACACAGAGTATTGAGGTGTTGGATAATGTCCAACAGCT
TAAAAATCTAGCCGATTGCATAAACTCCGAAATTGGTATCTTGTGTTGTG
CACTGCGTAAATTGGAG

Seq. ID No: 232

>TPH_5ATGTTGATTTCCTTTACGCTTAACCTGGTCCACCAAAAGAAGAA
CAGCGAGTTTGAGATTTTCCTAGACTGCGACAGTAACAGGGAACAACTAA
ACGAGATATTCCAGCTGCTAAGACCCCACGTCAATCTGATAACCATGAAT
CCGCAAGAGGACTTTAGCGTGGAAGAAGATGACATGGAGTCAGTACCCTG
GTTTCCCATCAAAATTAGCGATTTAGATAAAAGCGCAAACAGGGTGCTTA
TGTACGGCAGTGACTTAGACGCGGACCATCCTGGCTTCAAGGATAACGTA
TATAGAAGAAGACGTAAATATTTTGCCGACGTCGCGATGAATTATAAATA
CGGTGACCCAATCCCACACATCGAGTTCACTGAAGAGGAGGTGAAAACAT
GGGGGACTGTGTTCCGTGAATTAAATAAGCTACACCAAACCCACGCATGC
CGTGAATACCTAAAGAATTTACCTTTGTTAGTCAAGCACTGTGGGTATAG
GGAAGACAACATTCCACAACTAGAGGACGTTTCCAGATTTTTGAAAGAAC
GTAGTGGCTTTACCATCCGTCCGGTCGCCGGCTACTTATCCCCGAGGGAT
TTCTTAGCTGGCCTAGCCTTTAGAGTATTTCACTGCACTCAATATGTTCG
TCACTCATCAGATCCGTTATACACGCCGGAGCCCGACACCTGCCATGAGT
TGCTAGGCCATGTACCTTTGTTAGCTGAGCCCAGTTTTGCACAGTTTAGT
CAGGAGATAGGTTTGGCCAGCTTGGGCGCCTCTGATGAAGCTGTCCAGAA
GTTGGCCACCTGCTATTTCTTTACGGTGGAGTTCGGTCTGTGTAAACAAG
AAGGGAAACTAAAGGTGTACGGCGCTGGGCTTCTGTCTAGTATCTCAGAG
TTGAAACACAGCCTGTCAGGTAATGCAAATGTGAAACCGTTCGATCCAAT
GGTCACTTGCTCACAAGAATGCATCATCACAAGTTTTCAGGAAGTTTATT
TTTACTCTGAATCATTTGAGGAAGCTAAAGAGAAGATGAGAGAATTTGCC
AAGACAATAAAACGTCCGTTTGGTCTGAAATACAATCCGTACACGCAGAG
CGTACAAATGTTGAAGGATACCCAGTCTATCACTACGCTAGTATCTGAAC
TTCGTCACGAGCTGGATATCATCAGCGATGCTCTTAATAAAATGAACAAA
CAGCTAGGTGTC

Seq. ID No: 233

>TPH_6ATGCATTCCCCCGAACCTGATTGCTGCCACGAATTATTAGGGCA
CGTCCCGATGTTAGCTGATAAAACTTTTGCCCAGTTTTCTCAGGATATAG
GATTGGCATCCCTAGGTGTCACGGACGAGGAAATAGAAAAGTTGAGCACA
CTATATTGGTTTACGGTCGAGTTTGGTCTATGCAAGCAAGACGGTGAGGT
TAAAGCATACGGAGCAGGTTTACTGTCCTCATATGGCGAGTTACTACACG
CGCTATCCGACAAACCTGAGGTAAGACCTTTCGATCCAGACGAGGCTGCG
ATTCAGCCGTACCAAGATCAAAATTATCAACCAGTTTACTTTGTTTCCGA
GTCTTTTACCGACGCCAAGGAGAAATTGAGAAATTACGCCAGCAGGATTA
AGCGTCCCTTCGCGGCGAGGTATGACCCATATACAGTCTCCATCGAGGTG
TTGGACTCCCCTGGTCAAATTCAAAGTTCTTTGGAGGAACTTAAAGACGA
GTTGCAGACACTGACTACAGCGTTAAATATACTATCT

Seq. ID No: 234

>TPH_7ATGATGATATCTACTGAATCTGACCTAAGGAGGCAGTTGGACGA
AAACGTACGTTCCGAGGCCGATGAGTCCACTAAGGAGGAATGTCCATATA
TTAACGCAGTACAATCCCATCACCAGAATGTCCAGGAAATGTCCATAATC
ATATCGTTAGTTAAGAATATGAATGACATGAAGTCTATCATCTCCATTTT
CACAGATCGTAATATCAATATACTTCATATTGAAAGTAGGCTGGGCAGGC
TGAACATGAAGAAACATACTGAGAAGTCCGAATTTGAGCCCCTAGAGTTA
TTAGTTCACGTAGAGGTACCCTGCATAGAAGTCGAAAGGTTGTTGGAAGA
ACTTAAATCATTTAGTTCTTACCGTATCGTCCAGAACCCGTTGATGAATT
TACCTGAGGCGAAGAATCCAACCTTGGACGACAAGGTGCCGTGGTTTCCC
AGGCACATAAGTGACCTTGATAAGGTGAGCAATAGTGTTCTTATGTACGG
GAAAGAGTTAGATGCCGATCATCCCGGATTCAAAGATAAGGAGTACAGGA
AAAGGAGGATGATGTTTGCCGACATAGCTCTAAACTATAAGTGGGGCCAA
CAGATTCCCATTGTTGAGTATACTGAAATAGAGAAGACAACTTGGGGAAG
AATTTATAGGGAATTAACCAGACTATATAAGACGTCCGCATGTCACGAGT
TTCAGAAGAATCTAGGGTTACTACAAGATAAGGCGGGGTACAACGAATTT
GACCTGCCGCAACTACAGGTAGTCTCCGACTTTTTGAAGGCCAGGACTGG
ATTTTGTCTTCGTCCCGTTGCGGGGTATCTTAGTGCAAGGGATTTCCTTA
GCGGACTAGCCTTTAGGGTCTTTTACTGTACTCAGTATATTCGTCACCAG
GCCGATCCGTTCTACACACCCGAACCAGATTGCTGCCATGAGCTTCTTGG
ACACGTTCCGATGCTGGCGGACCCCAAGTTTGCTCGTTTCTCACAAGAAA
TAGGGCTTGCGTCACTAGGTACCAGTGATGAAGAAATTAAGAAATTAGCA
ACTTGTTATTTCTTTACGATAGAGTTCGGCTTGTGCAGACAGGACAACCA
GCTAAAAGCGTACGGTGCAGGTTTGCTTAGTTCCGTCGCAGAATTACAGC
ACGCACTTTCCGACAAAGCGGTTATCAAACCGTTTATTCCTATGAAGGTA
ATAAATGAGGAATGTTTGGTTACAACATTCCAAAATGGCTATTTCGAGAC
CAGTTCATTTGAAGATGCTACTCGTCAAATGAGGGAATTTGTCAGAACGA
TCAAGCGTCCTTTCGATGTCCACTATAATCCGTACACTCAATCTATTGAA
ATAATCAAAACCCCAAAATCTGTAGCAAAATTAGTACAAGATTTACAATT
TGAGCTAACCGCAATTAATGAGTCCTTGCTGAAAATGAACAAGGAGATTC
GTAGTCAACAGTTTACCACAAACAAGATTGTGACTGAAAATCGTAGTTCC

Seq. ID No: 235

>TrpHalo_1ATGTCTACAGCTTCAAAGAACATAGATATTACAAGATTCC
CCAAGAAATACGATGCGGCCACGAAAGACTCTGATTTTTATGATGTTGTA
ATCGTAGGTGCTGGCCCAGGTGGCAGTACAACTGCTTACTACTTGGCAAA
AGAAGGGAAAAAGGTTTTATTGCTTGAGAAGAAAAAGTTCCCCAGAGATA
AGATATGCGGTGACGCTATATGCAAACTAGCCATAGAGATGCTTATGGAT
ATGGGTGTCTACGAGGGATTAGTTAGGGAGAAGAAAGCTCGTGTAGCTCA
CAACGGAGGATTAGTTTCTCCCTCCGGCCTTAGTTTCATCGGGAATACGT
ACCTAAAGCCGGGGGAGATCCCTGCTGCAGCTGCGTGCAAACGTATGGTC
CTGGACGAAGCCATCGCTAAGGCCGCTATTGGGGCCGGAGCCGAACTAAA
AGAAAATTCCCCAGTGACAGACGCTGTCTTCGATAGTAGTACCGGGCTGT
GGACGATTAGCATTGAGGGCAGTGATGTGAAACATATGGGCCGTGTGTTA
GTATGTGCAGACGGTGCTCCTAGTAAACTAGCTACGCAATTAGGCATCGT
AAAACAGGCGCCCCAGGGGGTGTGTAGCAGAGCGTATATTAAGGAAGGTA
CACATAGATTCAGAGCGGACGGCGTCGTATTTTATCCGAGAAATATCTTA
CCAGCATATGCTGCCCTTTTCAGGCACATAGACGACACTGTCGCGTACTG
CACCTATATATTACCCTTTAACCCAAAAGTTACGACAGACGACCTGTCAT
ATTGGCATCATCGTCTATTAGAGGAGGACCCTTCTATTTCTCAAGCAGTC
GGTAAAAATGCTGATATGGAGAGAATGAAAGCCTGGGGTCTGCGTATGGG
AGGTGAACCCGTGACGTATGGTAACCATGTTTTGGTAGTTGGAGACGCAG
CTGGAATGATCGATCCACTAACAGGGGAAGGTATACACCACGCTATGGAT
GGCGGTCGTATCGCCGCACATTTCCTATGCGAAGCCATAGCGGTTGGGAA
TTTTGACAAGGAAGTCATGAAGGAATACCAGAACAGGTGGCTTTACACGT
TCGGAAATGACTACAAATGGAGCCAGGCCATATGCCACTTTTTATATCGT
TTCCCTATCTTCATCGATGCTACTGCCGCCGCCGCACAAAGGCGTGGGAA
CAACTTTCTTGCTCTTTACGCAGACATTATGACCGGGAGGATACCTAAGG
CCAATATTTTCCGTCCCGATATTAGTTTGCCCATAGCATTTGAAGTCTTG
GTACTATTGTGGAAGATGATGTTCACTGGGGGCGGAGGTAATAATAAAAT
GAAGTCACAG

Seq. ID No: 236

>TrpHalo_2ATGAGTACGGCGTCTAAAAATATCGACATCACCCGTTTTC
CGAAGAAGTATGACGCGGCGACCAAGGACAGTGACTTCTATGACGTCGTC
ATCGTCGGAGCAGGGCCAGGGGGATCAACGACGGCTTATTATCTAGCGAA
AGAGGGAAAGAAGGTACTACTTTTGGAAAAGAAAAAGTTTCCGAGGGACA
AAATTTGTGGAGATGCTATATGCAAGTTAGCGATCGAGATGCTTATGGAT
ATGGGCGTCTACGAGGGTCTGGTAAGGGAGAAAAAGGCAAGAGTTGCACA
TAACGGGGGTCTAGTTAGTCCAAGTGGTTTAAGTTTCATAGGCAACACGT
ACCTTAAACCAGGCGAGATTCCTGCCGCCGCAGCGTGCAAAAGAATGGTA
TTGGACGAGGCTATAGCAAAGGCAGCTATAGGGGCAGGGGCTGAACTAAA
GGAAAATAGCCCTGTGACTGACGCCGTGTTCGATTCTAGTACCGGTCTGT
GGACAATCTCTATCGAAGGTTCAGACGTAAAACATATGGGCAGGGTCCTA
GTTTGTGCGGACGGAGCACCCAGCAGACTTGCGATGCAATTAGGCATCGT
CAAGGGGACACCTAAATGCGTTTGCTCCAGAGCGTACATAAAAGGCGGGA
CCCACCGTTTTAAAGAGGACGGAATGGTATTCTATGTACCTTCTATCCTA
CCGGGGTACGTGGCTTTGTTGCGTCATATCGACGACCAACTTACATACTG
CACCTACATTCTACCAGGGAACCCAAGAGCAACTACGAAGGATTTGTCTT
ATTGGCATCATAGACTTCTTGAGGAAGACCCAAACATAAGCCAGGCAGTC
GGGAAGAACGCCGAACTTGAGAAGATGAAAGCCTGGGACTTACGTGTGGG
CGGTGAACCAGTAACGTACGGTAATCATGTCCTTGTGGTCGGTGACGCGG
CCGGTATGATAGATCCATTGACAGGTGAAGGCATCCACCACGCAATGGAT
GGGGGCCGTATTGCCGCTCACTTTTTGTGTGAAGCTATTGCAGTGGGTAA
TTTCGATAAGGAAGTTATGAAAGAGTACCAGAATAGATGGTTGAAGGCAT
TCGGAAATGACTTCAGATGGTCTCAAGCTATCGGCAATTTTCTTTACCGT
TATCCGATTTTCATCGACGCAACCGCCGCTGTCGCCGAGAAGAAAGGAGA
CAGGTTCTTAGCAAGGTGGGCAGATATTATGGCAGGAAGAATACCAAAGA
TTAGTGTCCTTAGGCCGCAGTTTTTGCTGGCTGTAGGCTTCCAAGCTCTC
CTACTATTCTATAAGAAGATTTTCAAAGGGGGTTATGGAAAGAAGACAAA
GATTCTA

Seq. ID No: 237

>TrpHalo_3ATGAGTAGCTTAATCGCGCCAAAAGTAGATACCATAGATA
TTACAAGGTTCCCCAAAAAGTACGACCCTGCAGCGGAGGACTCCGATTTC
TACGACGTAGTAATAGTCGGAGCGGGTCCCGGTGGCAGCACGACTGCGTA
TTACCTAGCGAAGAAGGGTAAGAAAGTGTTACTACTAGAAAAGAAGAAAT
TTCCTAGAGACAAGATCTGTGGTGACGCAATCTGTAAAACGGCAATTGAA
ATTTTAATGGATATGGGGGTCTACGGCGGACTGATCAGGGAACAGAAGGC
TTACATGATTGACTACGGGGGACTTGTGTCCCCTTCCGGTTTGAGCTTTG
TCGGCCACACCCATGAGCTTTTCGGTGAAATACCAGGAGCTGTTGTGTGC
AAGAGAGTTGTGTTGGACAAGGTAATATCAAGAACGGCACAATCAGCTGG
AGCTGAATTGTTGGAGAACTCCCCTGTAACAGATGCAGTTTTCGATTCCA
GCACTGGTCTTTGGACTATTTCTATTGAGGGCTCAGACGTGAAGCATATG
GGTAGAGTACTGGTATGCGCTGACGGAGCACCTTCCAGATTGGCCATGCA
ACTAGGAATTGTCAAGGGGACACCAAAGTGTGTTTGTAGTCGTGCCTATA
TTAAAGGCGGCACCCATAGGTTCAAAGAAGACGGCATGGTGTTCTATGTC
CCTTCAATACTTCCCGGATATGTCGCCCTTCTTCGTCATATAGATGACCA
GCTGACTTACTGTACGTATATCTTACCTGGAAATCCCAGAGCTACCACTA
AGGACCTTTCTTACTGGCACCATAGGCTGCTAGAGGAGGACCCCAATATT
AGCCAAGCAGTTGGTAAAAATGCCGAATTAGAGAAAATGAAGGCCTGGGA
CCTAAGAGTGGGCGGAGAACCCGTCACGTACGGCAATCATGTACTGGTAG
TAGGTGACGCTGCGGGCATGATCGATCCATTAACCGGAGAGGGAATCCAC
CATGCTATGGACGGAGGCCGTATCGCGGCTCACTTTCTTTGCGAGGCTAT
AGCTGTCGGCAACTTTGACAAAGAAGTCATGAAGGAGTATCAAAATCGTT
GGTTAAAGGCATTTGGTAATGATTTTAGATGGTCCCAAGCGATTGGGAAC
TTCCTATATAGATACCCGATATTTATAGATGCCACTGCGGCCGTGGCTGA
GAAGAAGGGTGACAGATTCCTGGCCCGTTGGGCTGACATCATGGCGGGTA
GGATTCCCAAAATATCCGTCTTGAGACCGCAATTCCTGTTGGCAGTAGGA
TTCCAAGCCCTTCTGCTTTTCTATAAGAAGATTTTTAAAGGGGGCTACGG
TAAGAAAACTAAGATCCTT

Seq. ID No: 238

>TrpHalo_4ATGTCTGGAAAGATTGACAAGATTTTGATTGTGGGAGGTG
GAACCGCGGGATGGATGGCTGCAAGTTATCTAGGGAAGGCGTTACAGGGC
ACTGCGGACATTACGTTATTACAGGCTCCAGATATCCCGACACTAGGGGT
AGGAGAAGCAACCATACCTAATTTACAGACCGCGTTCTTTGATTTCTTGG
GAATACCCGAGGATGAGTGGATGAGGGAATGCAATGCCAGTTACAAAGTT
GCGATAAAGTTCATCAACTGGCGTACGGCGGGCGAAGGTACCTCTGAGGC
GAGGGAATTAGATGGCGGGCCGGACCATTTTTACCACAGCTTTGGCCTTC
TAAAGTACCATGAGCAGATTCCTCTGTCACACTACTGGTTCGACCGTTCC
TACAGGGGCAAGACGGTTGAGCCGTTCGACTACGCGTGCTACAAGGAGCC
GGTTATTTTAGATGCTAACAGAAGCCCAAGGAGATTGGACGGCTCCAAGG
TCACTAACTATGCGTGGCATTTTGATGCGCACCTAGTCGCTGATTTTCTG
AGAAGATTTGCAACTGAGAAGTTAGGGGTAAGGCATGTGGAAGATAGAGT
TGAACACGTGCAGCGTGATGCCAATGGGAATATTGAGTCCGTGAGAACGG
CTACCGGAAGAGTATTTGACGCTGACTTGTTTGTAGACTGCAGTGGATTT
AGGGGGTTACTAATTAATAAGGCAATGGAAGAACCCTTTTTAGACATGTC
CGACCATTTGTTGAATGATTCAGCCGTAGCGACTCAGGTACCACACGACG
ATGATGCTAACGGAGTGGAACCTTTTACCAGTGCAATAGCCATGAAGTCA
GGTTGGACATGGAAAATTCCCATGCTGGGCAGATTCGGGACAGGTTATGT
GTATTCTTCCAGGTTTGCCACTGAGGACGAGGCAGTGCGTGAATTTTGCG
AGATGTGGCATTTAGATCCAGAGACGCAGCCTTTGAATAGGATTAGGTTT
CGTGTCGGGCGTAACCGTAGGGCTTGGGTCGGTAATTGTGTGTCCATTGG
AACTTCTTCTTGCTTCGTAGAGCCACTTGAGTCAACTGGAATTTACTTCG
TGTATGCCGCACTATACCAGCTTGTCAAGCACTTCCCTGACAAGAGTCTG
AACCCTGTTCTAACTGCTCGTTTCAACCGTGAAATTGAGACAATGTTCGA
TGATACCCGTGACTTCATTCAGGCACACTTTTACTTCAGTCCACGTACCG
ATACACCGTTCTGGCGTGCTAACAAAGAGCTGAGACTTGCAGATGGGATG
CAGGAAAAGATCGATATGTATAGGGCTGGCATGGCTATCAATGCGCCTGC
CAGTGACGATGCTCAGCTTTACTATGGAAATTTTGAAGAGGAGTTCCGTA
ACTTCTGGAACAACTCAAATTACTACTGTGTTTTAGCGGGATTAGGTCTT
GTTCCCGATGCCCCTAGCCCCAGGCTGGCCCACATGCCTCAAGCAACGGA
ATCAGTGGACGAGGTGTTTGGCGCCGTTAAGGACAGACAACGTAACTTGC
TTGAGACCCTACCATCACTGCATGAGTTCCTTAGGCAGCAGCATGGGAGG

Seq. ID No: 239

>TrpHalo_5ATGGACGAAATAGACGACCCCCGTATAAGGAGTGTAGTGA
TAGTGGGAGGCGGTACCGCGGGGTGGATGACCGCCGCCGCTTTAGTTCAG
CACTTTAGAACCGCGCCTCTAAAAATAACGGTGGTGGAGAGTTCCGATAT
TGGGACTATTGGTGTAGGTGAAGCTACGATTCCGACCATTCGTAGATTCT
ATGGCCAACTAGGACTTAGGGACGACGATGTGATGAGAGCTACGCAAGCG
ACATGTAAATTGGGCATCAGGTTCTTAGATTGGAGTGGACCTGGGTCCGA
CTTTATTCATCCCTTTGGTCTTTATGGCCAAGATGTTAAGGGCATTGGCT
TCCACCACTATTGGCTTAAACAAAGGCGTGCCGGAGATGCAGCGCCGTTG
GCTGCGTACAGCTTAGGAGCGGCACTGGCTGCCGGTGGCAAATTTACGTT
GCCCAGTCCTCATCCACCCTCTCAACTGTCTGTATTTGATTGGGCATTGC
ACCTTGACGCAGGCCTATTCGCCCAGCACCTAAGAGCGTACGCAGAAGCC
GGTGGCTGTGCCAGAATTGATGCCCGTATAAGATCAGTTGAGCTTAGACC
AGAAGACGGCTTTGTCAGAGCCCTTACATTGGACGACGGAAGAGAGGTAG
AGGGGGACTTGTTCGTGGACTGTAGCGGGTTCAAAGGACTTGTTATCGGT
GAGGCGCTGGGAGTTGGGTTCGAAGACTGGGGGAGGTGGTTACCTTGCGA
CGCAGCGTATGCTGTACAGTCTGAAAACAGGCCAGGCGATGCGCCAGCTC
CGTTCACACGTGTTACAGCTAGAAGCGCCGGATGGCAGTGGGGCATACCG
CTTAGACATAGGGCGGGTAACGGGTTAGTATTTTCAAGTGCCCATCTGTC
AGACGACCAAGCCCTTGCTGAGTTGATGCCGCATTTATTAGGAGACCCCC
TTACCGAGCCGAGGCGTATACCTTTCCGTCCCGGCCGTAGATCTCAAGCG
TGGGCAAAGAATTGTGTGGCTATTGGCCTTTCATCCGGCTTCTTAGAGCC
CCTGGAGTCCACTAGCATCGCTCTGATCGAGACAGGAATTGAACGTTTGA
AAGCCCTGTTCCCTGACCGTAGGTTCGCGCAACCTATCCTAGACGAATTT
AATGATCAAACGGCGAGAGAGATGGAGAGGGTTCGTGACTTTATCATACT
ACATTATAAACTTAATAGGAGAACTGACACCGACTTCTGGAGAGACTGCA
GGGAAATGCCCGTTCCCGAAACATTGGAGAGAAAGATAGCCCTATGGACG
GCAAGAGGACAGTTTGTACGTTATCGTTGGGAAATGTTCCATCCAGCAAG
CTGGTTAGCGATCTATGATGGTTTCGGCCTTTATCCCGATCACCATGATC
CAGCGGTGGATGCTATGGACCCCGCCTATTTAGCTAGGTCATTAGCGGAA
ATGAGAGCTAACATTGCGGACTTAGTTGCGCGTACCCCCGAACATGCTCA
GTTCCTTGCTGGGTTGGACCCGGCGGCTTCGGCCGCT

Seq. ID No: 240

>TrpHalo_6ATGATACGTAGTGTCGTGATTGTAGGAGGAGGAACGGCCG
GTTGGATGACGGCATCTTATCTAAAAGCTGCCTTCGATGACCGTATCGAC
GTAACTCTGGTAGAATCCGGTAACGTGCGTCGTATTGGAGTCGGGGAAGC
TACTTTCTCAACAGTAAGACACTTTTTCGATTATCTGGGGTTGGATGAGC
GTGAGTGGTTGCCACGTTGTGCTGGTGGATATAAATTAGGTATACGTTTT
GAAAACTGGTCCGAACCAGGGGAATACTTTTATCACCCATTCGAGAGGCT
AAGGGTAGTCGATGGTTTTAATATGGCAGAATGGTGGCTTGCGGTGGGAG
ATCGTCGTACTTCTTTCAGTGAGGCTTGCTACTTAACACATAGGCTTTGT
GAGGCCAAGAGGGCTCCAAGGATGCTAGACGGGAGTCTTTTCGCATCTCA
AGTAGATGAGTCATTAGGGAGAAGCACACTAGCAGAACAGAGAGCCCAAT
TTCCCTATGCTTATCATTTCGATGCCGACGAGGTGGCGAGATACCTATCA
GAATATGCAATAGCGAGGGGCGTGAGGCACGTAGTGGACGACGTACAACA
TGTAGGTCAGGATGAAAGGGGATGGATCAGTGGTGTTCATACTAAGCAGC
ACGGAGAGATTAGTGGAGATCTTTTTGTAGACTGTACAGGGTTCAGAGGC
CTACTTATAAACCAAACTCTGGGTGGTAGATTTCAGAGTTTTTCCGATGT
GCTTCCTAACAATAGGGCTGTTGCTCTGAGGGTGCCAAGAGAGAACGACG
AGGACATGCGTCCTTACACAACGGCTACCGCAATGTCTGCCGGTTGGATG
TGGACTATACCCCTTTTCAAGAGGGATGGAAACGGTTACGTGTATTCAGA
CGAGTTCATTTCTCCTGAAGAAGCCGAGCGTGAGTTGAGATCAACAGTGG
CTCCCGGCAGAGATGACCTGGAGGCAAATCATATCCAGATGCGTATCGGG
AGGAACGAGCGTACGTGGATTAATAACTGCGTGGCCGTCGGTTTGTCAGC
AGCGTTTGTAGAGCCATTGGAGTCAACCGGAATCTTTTTCATCCAACACG
CAATCGAGCAGCTGGTTAAGCACTTCCCCGGAGAGAGATGGGACCCTGTC
CTAATAAGTGCATACAATGAACGTATGGCACACATGGTTGACGGGGTGAA
GGAATTTTTAGTATTACACTACAAGGGTGCCCAGCGTGAAGACACTCCTT
ACTGGAAAGCAGCCAAAACCAGGGCGATGCCGGACGGACTGGCCAGGAAA
CTAGAGCTTAGCGCGTCTCACTTGCTAGACGAGCAGACTATATACCCCTA
CTATCATGGATTCGAAACCTACAGCTGGATCACCATGAACTTAGGGTTAG
GCATAGTGCCCGAGAGACCTCGTCCAGCTCTTTTGCACATGGACCCTGCT
CCCGCCTTAGCAGAATTTGAACGTCTGCGTAGAGAAGGAGACGAGCTAAT
TGCAGCCCTTCCATCATGCTACGAGTATCTGGCTAGTATTCAA

Seq. ID No: 241

>TrpHalo_7ATGTTAGAATCTATTGTGGTCGTAGGAGGTGGTACCAGTG
GATGGATGACCGCTTCATATCTGAGTGCTGCCTTTGGTGAGCGTATTTCT
GTAACAGTTGTAGAATCAGCGAGGGTCGGGACGATAGGGGTTGGCGAAGC
TACCTTCAGCACAGTAAGACACTTTTTCGAATACCTGGGGTTAAGTGAGG
AAACATGGATGCCAGCTTGCAATGCTACGTATAAACTAGGGATCAGATTT
GAAAACTGGAGAGCGCCAGGGCACCATTTTTATCATCCTTTTGAAAGACA
GAGGGTAGTGGACGGGTTCACCCTGCCAGACTGGTGGCTGGCCGATGGCG
GGGCCACCGAGAGATTCGATAAGGAATGCTTCCTGGTTGGCACACTGTGT
GACACTATGAGATCCCCTAGACACATGGACGGTGCTTTATTTGAGGGCGA
TCTAACGGACAGGCCCGCTGGACGTTCAACGCTGGCCGAGCAAGGAACGC
AGTTTCCTTATGCATATCATTTCGATGCGGCGTTGCTTGCCGACTTTTTA
CGTGATTACGCTGTGGCGAGAGGGGTCTTGCATGTTGTGGACGATGTGGT
TCACGTGGCTAGGGACGAAAGGGGATGGATCTCTCATGTTGCCACAAGGG
GATCTGGAGACCTGGCGGGCGATTTGTTTGTCGATTGTACTGGATTCAGA
GGATTACTTATTAACGACGCTTTGGACGAGCCGTTTGAGTCTTATCAAGA
CACCCTACCGAATGATTCCGCAGTGGCTCTGCGTGTACCAGTTGACATGG
AGCGTGAAGGCTTGAGGCCATGTACCACATCAACCGCTCAAGCGGCGGGG
TGGATATGGACTATTCCACTTTTTGGACGTGTTGGAACCGGCTATGTCTA
CGCGAGGGATTACTGCACACCTGAGGAAGCTGAACGTACGCTGCGTCGTT
TCGTAGGCCCGGCTGCCGACGACTTAGAGGCCAATCACATTCGTATGAGA
ATTGGAAGGAGTAGACGTTCATGGGTCAACAATTGCGTCGCAGTGGGTTT
AAGCAGCGGGTTCGTAGAACCGTTAGAAAGTACCGGTATCTTCTTTATTC
AGCACGCAATAGAACAACTTGTTAAACACTTCCCCGATGCGGACTGGGAC
CCCGCTCTGCGTTCCGCCTACAACACCTTGGTTAACAGGTGCATGGACGG
AGTAAGGGAGTTCTTGGTCTTGCACTATTACGGGGCAGCGAGAGCTGACA
ATGAATATTGGAGAGATACAAAGACGAGGAAAATCCCCGACTCTTTGGCG
GAGAGGGTGGAGCAGTGGCGTACCAAATTGCCCCATCCAGAGAGTGTGTA
CCCTCACTATCATGGTTTTGAAGCGTACTCCTACGTCTGTATGGTGCTTG
GACTGGGTGGTATTCCTCTAAAACCGAGCCCTGCCCTACGTATGCTTGAC
CCCTCCGCAGCCCAGCGTGAGTTCCGTCTGTTGGCGACTCAAGCAGAGGA
TCTTAGGCGTACCCTACCGTCTCAATATGCTTACTTCGCACAGTTTAGA

Seq. ID No: 242

>TrpHalo_8ATGAATAAACCTATAAAGAATATAGTGATCGTCGGAGGCG
GAACCGCAGGCTGGATGGCAGCGTCATATTTAGTAAGGGCCCTTCAGCAG
CAGGCCAATATAACCTTAATTGAATCCGCTGCTATACCTCGTATCGGTGT
CGGGGAGGCCACCATACCCAGCTTACAGAAGGTCTTCTTCGACTTCCTTG
GTATTCCCGAGAGAGAGTGGATGCCTCAGGTTAATGGAGCGTTCAAGGCA
GCTATCAAGTTTGTTAATTGGAGGAAAAGCCCGGACCCTTCCAGGGATGA
CCATTTCTACCATCTATTCGGCAATGTTCCGAATTGCGATGGCGTGCCCC
TAACGCACTACTGGTTGCGTAAGAGGGAGCAGGGATTTCAGCAACCGATG
GAGTACGCTTGCTATCCCCAGCCTGGGGCGTTAGACGGCAAATTAGCGCC
ATGCCTTTCCGATGGCACACGTCAAATGAGTCACGCTTGGCATTTCGACG
CTCACTTGGTAGCTGATTTTCTGAAGAGGTGGGCCGTTGAGAGGGGCGTC
AATAGAGTAGTTGATGAGGTCGTCGATGTCAGATTGAATAACCGTGGCTA
CATAAGTAATTTACTGACCAAAGAGGGGAGAACTTTGGAAGCGGACTTAT
TCATTGATTGCTCCGGGATGCGTGGACTGCTGATTAACCAAGCGTTAAAA
GAACCATTTATAGATATGTCCGATTACTTACTGTGTGATTCTGCCGTTGC
GAGTGCCGTACCCAATGACGACGCCCGTGACGGGGTTGAGCCCTATACGT
CTAGTATCGCGATGAATAGCGGCTGGACGTGGAAGATACCAATGTTAGGT
CGTTTCGGTAGTGGATACGTCTTTAGCTCACATTTCACGAGTCGTGACCA
AGCAACAGCGGATTTCTTAAAGCTGTGGGGACTATCTGACAACCAACCCT
TGAACCAGATTAAATTCCGTGTCGGGCGTAATAAGAGAGCCTGGGTAAAT
AATTGCGTCTCAATCGGGCTATCAAGTTGTTTCCTGGAACCGCTAGAATC
TACTGGGATTTACTTTATATACGCAGCATTATATCAACTGGTCAAACACT
TCCCGGATACGTCTTTTGATCCACGTTTATCAGATGCCTTCAACGCCGAG
ATCGTACATATGTTCGACGATTGCCGTGACTTTGTCCAAGCGCATTATTT
CACAACTTCGAGAGACGACACCCCGTTCTGGTTAGCAAACAGGCATGATC
TAAGACTTAGCGATGCGATCAAAGAAAAAGTCCAGCGTTACAAAGCTGGG
CTTCCTCTTACCACAACGAGTTTCGACGACTCTACCTATTATGAAACGTT
TGACTACGAATTTAAAAATTTTTGGTTAAACGGCAACTATTATTGTATTT
TTGCGGGTCTTGGGATGTTGCCGGATCGTAGCCTTCCCCTTCTGCAACAC
CGTCCCGAGAGTATAGAAAAAGCAGAGGCTATGTTTGCGTCCATCCGTAG
GGAGGCAGAAAGATTGAGGACCAGTCTACCTACAAACTATGACTACTTGA
GATCCCTACGTGATGGTGACGCGGGATTAAGCAGAGGCCAAAGGGGACCG
AAACTTGCCGCACAAGAGTCTTTG

Seq. ID No: 243

>TrpM_1ATGAGTCCCGTCGCACTTTCTCCTAAAAGGGTTGACATAGTAG
ATATTAGAGGGAACGACATGCAGTATAGTTTAGTCAACGAAATTCATAAA
GGTCTAAATCCTCCTAACGGTACCAGGAGAAGCCTTCCCACAATGCTATT
ATACGATAGTGAAGGGCTTAAACTATTTGAGAAAATTACTTACGTCGATG
AGTACTATCTGACTAACGCAGAGATTGAGGTCTTGGAAAAGCATTCCCGT
AGGTTAGTGGAGAAAATTCCGAGTAACGCGCAACTGCTAGAGTTGGGCTC
CGGTAATCTTAGGAAAATTGAGATCCTTCTGAGAGAGTTTGAGCGTGTAG
GTAAGCCCGTCGATTACTACGCATTGGATCTGTCACTGAGTGAATTAGAG
CGTACCTTCAGCAATGTCTCATTAGAAGAATATAAGTCTGTAGGATTTCA
TGGCTTGCACGGTACATATGACGATGCACACACTTGGCTTTCTGACCCGA
AAAATCGTGAGAGGCCCACTGTCGTTTTGTCAATGGGGTCTAGTCTAGGG
AACTTTTCTCCCCCTGATGCAGCTGCCTTCCTTGCTGGGTTTGCAACCCT
TTTGAAACCCTCCGACTTCATGGTCATTGGTTTAGATGCCTGTGAGGACC
CTGATCGTGTATATAAGGCTTACAACGACAGTGCCGGTATAACGAGAAAG
TTTTATGAGAACGGCCTTGCAAATGCAAATAAAACTCTGGGTCACGAGGT
ATTTAGACCTGACGAGTGGGAAGTGGTAACGGAATATGATGCGGTCAATG
GAAGGCATCAAGTATTCTATGTCCCTACCAAGGACGTAAGCGTGGGTGAC
GTATTGCTAAGGCGTGGCGAGAAGATTATCTTCGCTGAGGCATTTAAGTA
CGGATGCCAAGCACGTGAAAAGTTGTGGCACGACGCTGGACTGATTGAAG
CAGCCGAGTTCGGCTCCGGGAGTGAGGACTATAGGACTTATATATAA

Seq. ID No: 244

>TrpM_10ATGCTAGGACCAGTACCTTCTCCTAGTCCAGTCCCAATACCG
CCTGGCTCACGTCCTGGGGCAAGCCCCGGACTGGAGGCTACCATCCCCAT
CATAGACATACGTAGCACAGCTCACTCCGTTACAGTAGCAGCTCTTGAGG
ACGGTATCCGTGCTAACGTATTAAGCGGGTTTACAAAGCCTTACAATGAA
AAAGAGCTACCAAATCTTTTATTGTATAACGAGGAAGGATTAAGATTATT
TGAACAAATTACCTACCAGCCAGATTACTACTTGACCAGATTGGAAATCG
ATATTTTATCAAGGCACGCCCATCAGATTGCGAACTCAGTACCCGACGGT
GCCATCTTATTGGAGTTAGGGGCAGGTGCACTTAGAAAAACGGCGCTAAT
TCTGGACGCGTTGGAGGCGCAGGGCAAGGACGTTACATACTTTGCTTTAG
ACCTAGATAAACCGGAGCTGCTACGTACATTGGCCGAAGTCAAAGGGAGA
TATACGCACGTGAGCCTGGCTGGACTATGGGGGACGTACGATGATGGCTG
CACGTGGCTTAAACAAGTGAAAGATAGACCTCGTATTATTCTATGGTTGG
GTTCCTCAGTAGGAAACATGAGCCGTAAAGAAGCTGGCCAATTCATCAGA
ACCTTCGGCGATATTCTGGCGCCCAGGGACAGGTTCATTGTTGCTATAGA
CAGTAAGAACCATAAGTTGAACGACATTCGTGCCGCTTACGACGACCGTG
CGGGCGTTACCCGTAGATTCGCATTGAACGCGTTGGGAAACATTAATGAC
CTGTTCAATGCGGATGTTGTTGACGTCAGTTCTTTTGATTACAACCCGTA
CTATAATGAAGTACAGGGCAGAAACGAAGCGTATTTCAGGTGCCTTAAAG
ATACGCAAGTCCGTATCCCCTCCGAGACACCTATATTGGTACACGAGGGT
GAGTATATCAGATTCGCGTTTAGTCATAAATATGATAGAGTTGAAAGGCA
AGTTCTGTGGACAGCCGCAGGGGCATACCCCGTTCAGGAATGGATGTCAC
AGGACGGTGACTATGCACTGACAATGCTTAGTTGGTCAAGCTAA

Seq. ID No: 245

>TrpM_11ATGACCTACAGTATAGTTGATATTCGTAAAACCGATACTTGT
TTAAAGAATAGCATAATCAATGGCATTAACCAGTCCACTAAATCAATCCC
TGCTATTGTACTATACGACGAACTTGGCCTGCAATACTATGAAAAAGTGA
CCTATTTAAAGGAATATTATTTAACGGAAGCCGAGATTGACATCCTGAAG
AACAAGGCTGACCAGATCTCTGACTATATTCCGGAAGGCAGCTCCCTGAT
AGAGCTTGGGTCTGGGGCGCTTAGAAAGACAAGATTACTACTAGATTCTA
TCGAGAAGCAGAAAAAGAAGGTTATCTACTATGCCCTGGATTTAATGGAG
GGCGAATTGAAGCGTACGCTTTCCTCATTGGGCAAGTTTCAATATGTAAA
ACTTGTTGGCTTGTGGGGTGTTTACGAAGATGGCATTGATTATGCGTCTA
ATCTGCCAGGGGATTCACACAAAACTATTCTATGGATGGGAAGCAGTATC
GGCAACTTTAATCGTGACGAGGCTGCTAATTTCGTTAAGACGATCCAGGA
CAAAGCTATGAACCCAGGAGATCTATTCCTGATTGGGATAGACAGACGTA
AAAACCCGGACAAGATAACAGCCGCCTACAACGATCCAAAGGGAATCAAC
GCAAAATTCATAATGAACGGTTTGAACCACGTAAATGCAATATTCGACCA
ACCTATTTTCGATTCTAATAACTTCGAACATGTAACTATGTATAACGATG
ATGTTGGAAGGCATGAGGCTTACTGCAAGGTAAAGAATGATACAACTCTT
GAGTTTAAAGAATCCAAAGATAACCCGAAAACAATAATCAAACTTAATAA
GAATGAGTTAATTAACATAGGTTACTCCCATAAGTATAATAAGGCGGAAA
CAGATGCTCTATTTGACTTCTCACTACTAAGCTACATGGAGTCTTGGACC
GACTCACAGAGCCTATATGACCTGCATTTGGTTTATAAGAGTCCCTTCCA
CTTCACACGTAAGTTTGATAGCCACAAATAA

Seq. ID No: 246

>TrpM_12ATGTCTAAAGACGTGCAAGTTCTTGATATACGTGCAAGCCCC
CAGTCTAAGGGCTCAATTCCTAATCTGCGTACAGCGATATTGGATGGCTT
GCAGAAGGCGCCCGGTATGAGAACTCTACCTAGTGAAATTCTATATGACG
ACAGGGGCCTTAAAATCTATAATGACTGTATCAGATCTTGGTCTGAGTGG
TATTATCCCATCTCTGCCGAAACGGAAATCCTTGAGATCAACGGTAAAGA
CATAGCCAGGGTGTTCAGTACAAGTGATCGTGGTGAGGCGGTGTTAATTG
AGCTGGGTGCAGGGAGCCTGGATAAGACCAGCAAGATTCTAGTATCCCTT
TCTGAAACCGTTCAAAATGTATCCGATAGCCAGCCTCCGATAACTTATTA
CGCACTTGACCTGGAGCGTAGTGAACTGCAACGTACACTTAGTGAACTAC
AAAAGAACATCGGGGAGAAGATAGCGGGGAAAATTGCGACGAAAGGGATG
TGGGGTACTTATGACGATGGAATCCGTTCTGTTGAGAATAACGAACTACA
CCTGGACGCAGCAGTGCCGGTTCACTTTTTGTTCCTTGGTGGTACTATCG
GCAATTTCTCAAAAGGAGAGGGAGATGTGACCTTTCTTAGGAATCTGCCC
TTGAATGCACAGAGAGGAGATACGATTCTTTTGGGCATTGACCGTGAGAA
ATCCAAAGAGATTATCGAGAGAGCGTATAATTTTCCCGCCGCCCGTGAGT
GGATTATGAACGGTCTAAACGTCTCAGGGCACTTACTTTCTGGTGACAAA
GACCTGTTCCAATTAGATAACTGGGACCGTTATGCGATGTACGACGAAAA
ACTGGGCAGATTAGAAGCGGGATATCGTAGCAAGATCGATCAAATCATCG
AAGTGACGGCGAATTACAGCATACCTTTTAAGAAGGATGAATCAGTGATG
GCCATTTTCTCCAATAAGTACACCGACGATGAACTTAATTTCCTTATTAG
CAAGGCCAATCTAAAGACAATCAACAGTTGGGTCGATCACAAGGCCTTGT
ACTATATTTTCTCACTGAGGAAGGTCTGA

Seq. ID No: 247

>TrpM_13ATGCCGCGTATACAGGTGCTAGATATAAGAGGCAGTAAGGAG
AGCGTGGGCAGTACGCCACACTTACGGGCTGCGATACTTGAAGGGTTACT
GAAACCACCCGGTTCCCGTACCCTGCCGAGCGAGACTCTATATGATGAAG
TCGGACTGAAGATGTACAATGATGGCATGAAGGCCTGGGCTGAATGGTAT
TATCCCGTAGAAGCCGAACGTCAAATCTTGGAAAGATATGGGCGTGATAT
TGCAAAGCTGTTCACAACTTCTGCCAAGGGGAAAGCGGTACTTATAGAAC
TGGGAGCCGGGTCACTTGACAAAACCAGTCAGGTGCTGCTAAGCGCGGCA
GAAATCACACGTACCACGGGACCCATGAACAATATAGCATATTACGCATT
GGATTTGGAGCGAGGGGAGCTGGAGAGAACCATAGGTCGTTTACAGGAAG
TCATAGGCGATCAAATAGCAGGTAAAATCAGTACAGCCGGTATGTGGGGA
ACCTATGATGACGGGATTAGGGTAATTGAGAAAAACGAACTTGAGCTAGA
ACCGGACATCCCTGTGCACATTCTGTTTCTCGGGGGCACTATCGGAAATT
TCTCTAAGCAGGACGGAGACGTCGCCTTTCTTAAATCCCTGCCACTGGAC
CATAAAAGGGGAGATACGCTTTTAGTAGGGATGGATAGACACAAGAGCGC
AGACGCCATCGAGAGGTCCTATGGATTCGCCGCGGCGAAGGATTGGATCA
TGAACGGCCTTAAAGTATCAGGTAGAGTTCTTACCGGGGACGAAGGTTTA
TTTGAAATAGGGAACTGGGAGAGATACGCAAAATACAATGAAGAACTGGG
CAGGTATGAAGCAGGATATAAAAGTCAGAAAGAGCACGCCTTAAAAATCA
GTGAGGGAGTAGACATCACCTTCTTAAAAGATGAGGTAGTGTTGGTTATG
TTTTCTAATAAGTACACCGACGCGGAAATGGACAGCGTAGTCGATTCTGC
GGGCCTAGTTAAAAACGGCTCCTGGATGGATGAAAAAGCTCAGTATTGCC
TGTTATCTCTGAGAGCCAACAACGGCCCCGTCTAG

Seq. ID No: 248

>TrpM_14ATGAGCCAGATAGAAGTTCTGGACATTCGTGGAAGCAAGGAA
GCTACAGGTTCTACACCACACTTGAGGGCGGAAATTTTGCAAGGTCTTTC
AAAGTCACCTGGGCATCGTACCATTCCCGGTGAGACCTTATTTGATGAGA
CCGGGTTGAAGATGTATGACGAGGGGATGAAGACTTGGAGAAAGTGGTAT
TACCCGTTCGAGGCTGAGAAGGAAATCCTTGAAGTTAGGGGTCTGGAGAT
AGCCAAATTGTTGAAAACCAGTTCCAAAGGGGAAGCCGTACTAATTGAAT
TAGGCGCTGGGTCTTTAGAGAAAACTTCACAAATACTTTTATCTGCCGCT
CAGATAGCCGAAACAGCCGATAACAGCACTACTAACCCCATTACATACTA
CGCACTAGACCTGGAACATAGAGAGCTGGAGAGAACACTGGCGGCCTTAC
AAGACGCTATCGGTCCGAGAATCGCTGGTAAAATCACAACCAAAGGGATG
TGGGGGACCTACGAAGACGGAATTAGAGTCGTGGAAAGGAATGATCTAAA
ATTTCCTTCAGATGTACCTTTACACATCCTATTTCTAGGAGGAACCATTG
GCAACTTTTCTAAAGCTGATGGCGATATTGCATTTTTGAAGAGCCTGCCT
TTGAATCGTAAGAGGGGAGATACATTGCTATTGGGAGTGGACAGAGCTAA
GGCGGTTGAACTAATTGAAAGAGCCTACGGCTTCGCGGCTGCTACAGGTT
GGATAATGAACGGCTTAAAGGTAAGTGGAAGGGTTCTAACCGGAGATGAA
GAGCTGTTCGAGAGCGGGAATTGGGAAAGGTACTCCAAGTACAACGAGGA
ACTAGGAAGATATGAAGCAGGATATAAGAGCAGGAAGGATCAAACAATAA
AGGTTGCTAAGGATGTAGACATCGTTTTTAGTAAGGATGAAGTCATTCTA
GTTACTTACTCAAATAAGTATACTGATGCTGAAATAAAGACGGTCTTTGA
CGGTGCCGGACTGGAAATTGTGGAATCTTGGATGGACAAAAAGGCACAGT
ATTGCTTGTTTTTACTAAAAGCCTGA

Seq. ID No: 249

>TrpM_2ATGACGTTGAGTCTTGCTAATTACCTAGCGGCGGATAGCGCCG
CCGAGGCTTTAAGGCGTGACGTGAGGACCGGTCTGACGGCGACACCAAAG
AGTCTTCCTCCCAAATGGTTCTACGACGCTGTAGGTTCCGACCTTTTTGA
CCAAATAACTAGACTACCCGAATATTACCCTACCCGTACTGAAGCACAAA
TTTTACGTACCCGTTCAGCGGAAATAATCGCGGCTGCCGGGGCGGATACG
TTGGTCGAGTTGGGCTCAGGGACTTCTGAGAAGACTCGTATGCTGTTGGA
CGCAATGAGAGATGCGGATCTATTGAGGAGGTTCATTCCCTTCGACGTCG
ATGCTGGGGTATTAAGATCTGCGGGAGCGGCCATTGGGGCGGAGTATCCA
GGTATAGAAATTGATGCAGTCTGTGGGGATTTCGAAGAACATTTGGGGAA
GATCCCAAGAGTAGGTAGAAGGTTAGTTGTGTTTTTAGGTAGTACGATCG
GTAACCTGACACCACAGCCGAGGGCTGAATTTTTAGCCACACTAGCAGAT
ACTTTACAACCAGGAGACTCCCTGTTGTTAGGAACGGATCTTGTAAAGGA
TACTGGGAGACTTGTAAGAGCATACGATGACGCGGCTGGCGTAACAGCTG
CGTTTAATAGGAACGTACTTGCAGTCGTTAATAGGGAACTGTCCGCCGAC
TTTGATCTTGATGCGTTCGAACATATAGCTAAGTGGAACGATGACGAGGA
GAGAATCGAAGTTTGGCTACGTGCCAGAACAGCCCAGCATGTCAGAATAC
CTGCACTAGACCTGGAAATAGATTTCGCAGCCGGGGAGCAGATGCTGACC
GCAGTAAGCTGTAAGTTTCGTCCCGATAGTGTTGCCGCTGAACTAGCCGA
AGCAGGTTTGAGACAAACACATTGGTGGACCGATCCAGCGGGCGACTTCG
GACTGAGTCTAGCTGTGAGATAG

Seq. ID No: 250

>TrpM_3ATGACACTTAGCCTTGCAAATTACCTTGCGGCCGATTCAGCGG
CCGAAGCACTAAGGAGGGACGTCAGAGCCGGATTAACTGCTGCCCCGAAA
TCTCTTCCACCTAAGTGGTTCTATGACGCCGTAGGAAGCGATCTTTTCGA
CCAGATTACCAGATTGCCCGAGTACTATCCAACAAGAACAGAAGCTCAGA
TACTAAGGACTAGGAGCGCGGAAATAATAGCGGCGGCTGGAGCAGACACA
CTTGTAGAACTAGGTAGCGGTACTTCCGAGAAGACACGTATGCTACTAGA
TGCAATGAGAGACGCTGAGTTATTGCGTAGGTTCATTCCGTTCGACGTGG
ACGCGGGAGTGTTAAGGAGTGCTGGGGCGGCAATTGGTGCGGAGTATCCT
GGCATCGAGATAGACGCTGTTTGCGGAGATTTTGAGGAACACCTGGGTAA
GATTCCCCATGTCGGTCGTCGTTTGGTGGTCTTCTTAGGGAGCACCATAG
GCAATCTGACGCCTGCGCCCAGAGCTGAATTTCTAAGTACGTTAGCCGAT
ACCCTTCAACCAGGGGATAGCTTGTTATTGGGTACTGACCTAGTCAAGGA
TACCGGACGTTTGGTTAGAGCTTACGATGATGCGGCAGGAGTGACGGCCG
CTTTTAATAGAAATGTACTTGCTGTGGTTAACAGGGAACTATCCGCCGAC
TTTGATCTGGACGCGTTTGAGCATGTCGCCAAATGGAACAGCGATGAAGA
GAGGATTGAAGTCTGGTTGAGGGCGAGGACTGCTCAACACGTGAGGGTTG
CAGCTCTGGATCTGGAAGTGGATTTTGCGGCTGGAGAGGAGATGCTGACC
GCCGTTTCCTGTAAATTTAGACCTGAGAATGTCGTAGCCGAGCTTGCGGA
GGCAGGCTTGAGACAGACTCATTGGTGGACAGATCCAGCGGGAGACTTTG
GGTTGAGCTTGGCCGTTCGTTAG

Seq. ID No: 251

>TrpM_4ATGAGGGTCAGTGGGGCAAACCATCTGGGCGAGGATGCCGGGC
ACCTGGCATTAAGGCGTGATGTATATAGTGGGCTTCAGAAAACACCTAAA
AGTCTACCACCCAAGTGGTTTTACGATACCGTTGGCTCAGAACTATTTGA
TCAGATCACGAGATTACCGGAGTATTATCCCACGAGAGCCGAGGCCGAAA
TATTGCGTGCGAGGAGCGCAGAGGTAGCTTCAGCTTGCAGGGCAGATACT
CTAGTCGAGCTTGGGTCAGGCACGTCAGAAAAGACCAGGATGCTGCTAGA
TGCGCTAAGGCATCGTGGGTCCCTGAGGAGATTCGTACCTTTCGATGTAG
ATGCTTCCGTGCTTTCCGCCACTGCGACTGCGATTCAAAGAGAATATTCC
GGCGTGGAGATCAACGCCGTCTGTGGGGATTTTGAAGAACATCTAACCGA
GATACCCAGGGGCGGGAGGCGTCTATTTGTGTTTTTGGGCTCCACGATTG
GTAACCTGACACCGGGTCCCCGTGCCCAGTTTCTAACTGCCCTGGCAGGC
GTCATGCGTCCAGGGGACTCCTTGTTATTAGGCACAGATTTAGTGAAAGA
TGCTGCAAGGTTAGTGAGAGCTTATGATGATCCAGGAGGAGTGACGGCGC
AGTTTAACAGAAATGTCCTGGCCGTCATAAACCGTGAGCTAGAAGCAGAT
TTTGACGTAGACGCATTTCAACACGTAGCCCGTTGGAACAGCGCCGAGGA
AAGGATTGAGGTCTGGCTTAGGGCGGACGGAAGACAACGTGTGAGGGTGG
GTGCTTTAGATTTGACAGTAGATTTTGATGCGGGTGAAGAGATGTTAACT
GCTGTGTCATGTAAGTTTCGTCCACAGGCAGTTGGTGCAGAGCTTGCAGC
AGCCGGCTTACATAGGATTAGGTGGTGGACCGATGAAGCCGGTGACTTCG
GTCTAAGCCTCGCCGCAAAATGA

Seq. ID No: 252

>TrpM_5ATGACGCTGACATTATCAAATTATCTTGCTGCAGATTCTGCCG
CCACTGCTCTAAGGCGTGACGTGCATGAGGGGTTGACCCAGTCTCCGAAG
ATGCTTCCGCCGAAGTGGTTTTATGATAGCGTTGGGTCAGATCTTTTCGA
CCAAATTACCAGACTACCTGAGTACTACCCTACAAGAACGGAAGCGCAGA
TACTTACCCATCGTTCTCCAGAAATTGTCGCGGCAGCGGGGGCCGATACA
CTTGTGGAATTAGGATCAGGAACATCTGAGAAGACCAGAATGCTATTAGA
TGCGATGAGAGACGGGGGTCAGCTAAGACGTTTTATTCCGTTCGACGTTG
ACGCGGGCGTGCTAAGAGCAGCAGGGGCGGCTATAGGACAAGAATATCCA
GGGATTGAAATTGACGCGGTGTGTGGGGATTTTGAGGAACATTTAGGGAA
AATACCTGCGGTCGGCAGGAGACTGGTGGCTTTCTTAGGGTCTACGATCG
GTAACCTAACCCCAGGTCCACGTGCGGACTTTCTGGCAAGTCTTGCCGAG
ACTCTTCAGCCCGGCGATTCCGTGTTGCTTGGGACGGACCTGGTTAAGGA
CACTGGTCGTCTAGTCTCTGCATATGACGATTCCGCAGGAGTTACAGCAG
CGTTCAATAGAAATGTCCTTTCTGTAGTTAACAGAGAATTGGACGCTGAT
TTTGACCTAGATGCTTTCGCACATGTCGCTAAATGGAACGCGGAAGAAGA
GCGTATCGAGGTATGGCTTAGGGCCGACGCACCCCAGCAAGTAAGGATAG
CTGGGCTAGACTTAGACGTTGCTTTCGGGGCTGGCGAGGAGATGTTGACG
GCAGTTAGTTGTAAGTTCCGTGCTGATGGTGTCGCGGATGAATTAGCGAA
GGCCGGGCTGAGGCAAACACACTGGTGGACCGACGAGGCGGGTGACTTTG
GTCTAAGTCTTGCGGTCAAGTAG

Seq. ID No: 253

>TrpM_6ATGCTTGAAGCGACGTCTACCCAAAATCTGGTGAGCTTCCAAA
TACCCATCGTTGATATTAGGACACCGTCTTGTTTAGAGGAAACTATTAGG
AAGAAAGTAGTATCCGGTTTAGCCCGTCCTTATAATAAGAAATCTATTCC
GGACTTGCTACTGTATAATGAGACAGGCTTACGTCTTTTTGAGGACTTGA
CATACCAGCCGGATTACTATCTAACCGGACTGGAAATCGAAATATTGTCA
AAGCATTCTCTTCAGATAGCCGACTCAATCCCGGTAGGGTCCCTGATAAT
GGAACTAGGTGCTGGCGCCCTTCGTAAAACGGCCCTAATACTAGACGCCT
TAGAGGCACAGAAAAAGGAAGTTGCTTATTTAGCACTGGACCTGGACCGT
CCTGAGTTGGTGAGAACTCTTGGACAGTTGAACGGGAAATACACACATGT
GAAGCTGGGCGGGTTATGGGGTACGTATGATGATGGACGTCGTTGGCTTA
GTGAAAACACCAGTGATTCCCCCAGAACAATACTGTGGCTTGGAAGCTCT
ATTGGTAACGTCAAAAGAGATGACGCGGGGGACTTTATTCGTTCCTTTGG
TGACGTCCTTAGTTCTAAAGATCGTTTTGTGGTGGCAATAGATTCTAGGT
ATCACGAGGTTGATACCATTTGTAGGGCGTATAACGATAGAGAGGGTTTC
GCTGAAAGATTTTGCTTGAACGGAATAGACTCATTTAACCAACTATTCGG
CCGTGCTATCATTGACATCTCTTGCGCGAAATACCGTACAGTTTATAACG
AGGTCAAAGGGAGACACGAGGTGTACTACAGATGCACCCACGATTTCGAA
ATCAGGCTACCTGGAGATTATCCACCCACCTTCTTGTACGAGGGAGAACT
TATCCTGTTGGCGCATTCATATAAATATGCGGCGGTTGAGAGAGAGACGC
TATGGCTGCGTGCAGGAGCTAGACCGGAGAAAGAATGGATGACGGACGGG
AGCTATACCGTGACAATGTTGTCATGGCCATAA

Seq. ID No: 254

>TrpM_7ATGTCTCCATCTACGGTTAACAAAATCGCTTCTAGCCCGGTTT
TCGACATTCGTTCCGACGAAACGAAAGGCTTTGCTAAGGCTCCGATAGAA
GATGAGTTAGCAGGGCTACAAGCAGTATACAACGAGAAAACGCTGCCAAA
TGTACTTCTATATGATGCCAAGGGGCTGCAACTTTTCGAAAAGATAACAT
ACACCAATGACTACTACTTAACTGGACTGGAGATGGACCTTCTTGGAGAA
CACGCCGATGAGATGGCAGAATGGATAAAGGATGGAGCGGCTCTGGTCGA
ACTTGGTGCGGGCGCACTTAGGAAAACCGCTATCTTACTGGATGCCATTG
AACGTCAAGGTAAGAGGATAACCTTTTATGCGTTGGACCTAGACCACTCA
GAGTTGACCAGGACATTGGCGGAGCTAGAGGGTAGGTATAGGCACATAAC
ACTATGTGGCTTGTGGGGAACGTATGATGATGGCAGGGCCTGGCTTGCAT
CTACAAATGAAGAGCAGCGTGTCCTATTGTGGTTGGGTAGCAGTATTGGC
AATCTGTCAAGACAAGAGGCCAAGGATTTTCTGCATTCTTTCGGACGTGC
CTTGAGACCTGGAATCGACAAGTTTATAGTAGCAATGGACAGTAAGTATA
ACGCTGTCTCTTCCATGACCAGGGCATATAACGACTCAGAAGGAGTAACA
GCAAGTTTCGCGTTAAACTTACTTGACGCATTCAATGCTAAAGTCGGATT
CAAAGCACTGCCCCCATCATCCTTTTGCTATAGTCCCTTCTTTAATCAGG
CACAGGGGAGAAACGAGGCATATCTAAGGGCACGTCATGGTGTGAGGTTC
GAGGTTAACGGCATAGCAGTCGAAGTGAGAGATGAGGAGTTGATTCGTTT
CGCGTACTCCCATAAATACGATAACGCTGAGCGTGACTTGCTTTGGAGAG
CTGCTGAAGCAAATGTAGAACAAGAGTGGCTACATTCACCCCAGTCAGGT
AGGGCACGTTATTCCATAAGTCTGCTGTCCTTCCGTGATTAG

Seq. ID No: 255

>TrpM_8ATGACCCTGTCTCTATCAAACCATCTTCCAGCCAACAGTGCAG
CAAGGGTACTGAGGAGAGATGTCCTGGATGGTTTAACGCAAACGCCCAAA
GCCTTGCCGCCTAAGTGGTTTTACGACTCAGTCGGAAGTGATCTTTTCGA
CCAGATCACAAGACTTCCAGAGTATTACCCGACTAGAACAGAAGCACAGA
TCTTGAGGACCCGTAGTGCGGAGATTGCCGAGGCTTCTGGAGCCGATACA
TTGGTAGAGTTAGGGAGTGGGACAAGCGAAAAGACGAGGATGCTTCTGGA
TGCCTTGAGAGATAACGGTACTCTTAGAAGATTTATACCTTTTGATGTGG
ACGCTGGGGTACTAAATGCAGCCGGAGCGGCCATCCAGAAGGAATATCCA
GGCGTAGAGGTTGATGCAGTATGCGGCGACTTCGAGGAGCACCTTGGCGA
AATCCCTCGTGTCGGGAGGCGTCTAATCGCGTTCTTGGGGTCCACTATTG
GAAACCTTACACCCCAACCGAGAGCCCGTTTTCTGACGGCCTTAGCGCAA
ACCATGCGTCCAGGGGACAGCCTGCTTCTTGGAACTGACCTTGTCAAAGA
CACAGAGAGATTAGTAAGGGCATACGACGATTCAGCGGGAGTTACCGCGC
GTTTTAACAGGAATGTACTGGCGGTAATCAACAGGGAACTGGATGCGGAT
TTTGATCTAGCCGCTTTCGATCATGTCGCTAGGTTCAATGCAGCCGAGGA
ACGTATTGAGGTGTGGCTGCGTGCACGTGGGGCGCAAAGGGTGTATGTCA
GAGAACTTGACTTGACGGTAGACTTTGCCGATGGAGAAGAAATGTTGACC
GCGGTGAGTTGTAAATTCAGGCCCGATGGAGTTGCAGCTGAGTTAGCCGC
AGCGGGTCTGCGTAGAACTCACTGGTGGACCGATCCTGCTGGAGATTTCG
GCCTTAGTTTAAGCACGAAATAA

Seq. ID No: 256

>TrpM_9ATGACCATTTCCATAGCCAACTATCTGGCTGCCGACTCAGCAG
CTACAGCCCTGCGTAGAGACGTGAGAGAGGGATTGGCAGGGACCCCTAAG
TCTCTACCGCCCAAATGGTTTTATGACTCAGTTGGCTCCGACCTTTTTGA
CCAGATCACCAGATTGCCGGAATACTATCCTACTAGGGCAGAGGCCCAGA
TACTAAGAACACACGCGGTTGACGTAGCTGCGGCCTCTGGCGCTGACACG
TTAGTTGAGCTTGGCAGCGGGACCAGTGAGAAGACGCGTTTATTACTTGA
TGCTTTGCATCGTGCTGACAGTTTGAGGCGTTTCATACCGTTCGATGTAG
ATGCGAGTATCCTGCAGTCCGCCGGCGCTGCTATCAGTCAGGAGTACCCA
GATGTCGAAATTGAAGCTGTCTGTGGCGACTTTGAGGAACACCTGGGAAA
AATACCTCTTCAGGGTAGAAGGCTGGTTGTTTTCCTGGGATCTACTATTG
GCAACCTAACTTCCGGACCCAGAGCAACTTTCCTATCCGCATTGGCAGAT
TCCTTGCAACCGGGTGACACTCTATTGCTGGGTACCGATCTAGTGAAGGA
CGTAGATAGGTTGAAGAGAGCCTACGATGACGCAGCGGGAGTGACGGCAA
GGTTCAATAAGAACGTCCTTACTGTAGTCAATAGGGAGTTGGGCGCGGAC
TTCGACCTGGACGCATTTGAGCACGTCTGCAAGTGGAACGCGGATGAAGA
ACGTATCGAGGTTTGGTTGAGGGCTAATACTCTGCAACGTGTCCATATAT
CAGGACTGGAACTAGATGTAGAATATGCTGCGGGGGAAGAGATGTTGACA
GCAGTCTCTTGTAAGTTCCGTCCTGAGGGGATCGCGGCCGAATTAGCCGC
AGTCGGATTAAATCGTACCCACTGGTGGACGGATGACGCGGGAGATTTCG
GCCTTAGCTTAGCAGTAAAGTAA

Seq. ID No: 257

>TrpS_1ATGACGACGTTATTAAATCCCTACTTTGGTGAGTTTGGAGGTA
TGTACGTACCTCAGATTTTGATGCCTGCTCTAAATCAACTTGAGGAGGCA
TTTGTGTCCGCACAAAAAGATCCGGAGTTCCAGGCTCAATTTGCCGATCT
GCTAAAGAATTATGCAGGGAGACCGACGGCGCTTACTAAGTGCCAGAACA
TCACAGCCGGGACCAGGACCACACTATACCTTAAAAGAGAAGACCTGCTG
CATGGTGGTGCCCATAAAACGAACCAGGTTTTAGGCCAGGCTCTTTTAGC
CAAACGTATGGGAAAAAGCGAAATCATAGCAGAAACGGGAGCTGGGCAGC
ATGGTGTAGCCTCTGCCTTAGCCTCCGCTCTATTAGGCCTGAAGTGCCGT
ATCTACATGGGGGCAAAAGATGTGGAGAGGCAGAGTCCCAATGTTTTTCG
TATGCGTCTGATGGGAGCGGAAGTCATCCCAGTACACTCTGGGTCAGCTA
CGGTAAAAGACGCGTGCAACGAGGCGTTGCGTGACTGGAGTGGGTCATAC
GAGACAGCCCATTATATGCTTGGGACAGCAGCTGGCCCGCACCCGTACCC
TACGATAGTAAGGGAATTTCAAAGGATGATTGGTGAAGAGACAAAAGCTC
AGATATTAGATAAAGAGGGAAGGCTGCCTGATGCGGTGATCGCATGCGTT
GGAGGGGGTAGTAATGCCATTGGCATGTTTGCTGACTTTATCAATGACAC
AAGCGTTGGCCTAATTGGTGTCGAACCGGGCGGGCATGGCATCGAGACTG
GAGAACATGGTGCCCCACTGAAACACGGCAGAGTTGGGATATACTTTGGG
ATGAAGGCACCTATGATGCAGACGGCCGACGGTCAAATAGAAGAGTCATA
CAGCATAAGTGCGGGGCTAGATTTTCCAAGTGTTGGTCCACAACATGCCT
ATCTGAACTCCATCGGGAGAGCGGATTATGTTAGCATTACTGACGACGAA
GCACTAGAGGCCTTTAAGACCCTGTGCAGGCATGAGGGCATAATTCCGGC
ATTGGAAAGTAGTCACGCTCTAGCGCACGCGTTAAAGATGATGAGAGAAC
AACCCGAGAAAGAACAATTATTAGTAGTGAATCTTTCAGGCAGGGGCGAT
AAGGACATATTCACAGTACATGACATCTTGAAGGCTAGAGGGGAAATA

Seq. ID No: 258

>TrpS_2ATGTGGTTTGGAGAATTTGGAGGCCAATATGTCCTAGAAACAC
TAATAGGACCACTAAAGGAGTTAGAGAAAGCATATAAGCGTTTTAAAGAC
GACGAGGAGTTCAATCGTCAGCTTAACTATTACCTGAAGACCTGGGCAGG
AAGGCCAACGCCATTGTATTATGCTAAGAGACTTACCGAGAAGATTGGCG
GCGCTAAGGTCTACTTAAAGAGGGAGGACCTGGTCCACGGGGGCGCACAC
AAAACAAATAACGCCATAGGTCAGGCCCTACTAGCTAAGTTTATGGGAAA
AACTAGGTTAATTGCCGAAACAGGAGCAGGACAGCACGGCGTTGCCACAG
CGATGGCTGGGGCTTTGCTTGGAATGAAAGTAGATATATACATGGGAGCG
GAAGACGTTGAACGTCAGAAAATGAATGTTTTTAGGATGAAGCTATTGGG
CGCTAATGTCATACCAGTTAACAGCGGTTCCAGGACACTGAAGGACGCAA
TCAATGAAGCATTACGTGATTGGGTGGCGACGTTTGAGTACACACATTAT
CTGATAGGCAGCGTCGTTGGCCCTCATCCCTATCCGACAATAGTCAGGGA
TTTTCAATCTGTAATAGGTAGGGAGGCCAAAGCTCAAATACTAGAAGCTG
AAGGGCAATTGCCTGATGTCATCGTTGCTTGTGTCGGGGGAGGCAGTAAC
GCTATGGGGATATTTTATCCATTTGTTAACGACAAAAAGGTTAAATTGGT
AGGTGTCGAGGCAGGGGGAAAAGGTCTGGAAAGCGGTAAGCATAGTGCCA
GTCTAAATGCTGGGCAGGTAGGTGTTTCACACGGCATGTTGTCTTATTTT
CTCCAAGATGAGGAAGGCCAAATCAAGCCGTCTCATAGCATAGCACCAGG
GTTAGACTACCCGGGGGTCGGGCCCGAACACGCGTATCTAAAGAAAATCC
AGAGGGCGGAGTACGTTGCGGTAACAGACGAAGAAGCGTTAAAGGCCTTT
CACGAGCTATCTAGGACAGAAGGTATTATTCCAGCCCTAGAATCCGCGCA
CGCGGTGGCATACGCTATGAAGTTAGCCAAGGAAATGAGTCGTGACGAAA
TTATTATAGTGAACCTATCTGGGAGGGGCGACAAGGATCTGGACATAGTA
TTAAAAGTGTCAGGGAACGTA

Seq. ID No: 259

>affibody_tag_1ATGGTGGACAACAAGTTCAACAAGGAGACGATACA
GGCATCACAAGAAATAAGGCTGTTACCTAATCTTAACGGTCGTCAAAAAC
TGGCGTTCATTCACTCACTGTTAGACGACCCCAGCCAGTCCGCGAACCTG
CTGGCGGAAGCTAAAAAGTTAAATGATGCACAAGCTCCGAAGAATGCCGC
AATCCGTTCTTCCTCTGCTTCATCTGGAGGGAGCGGGGGCAGCTCAAGTA
GC

Seq. ID No: 260

>affibody_tag_2AACGCAGCAATTAGAAGCAGTAGTGCTAGTAGTGG
TGGATCAGGAGGTTCCTCATCCTCCGTGGATAATAAGTTTAACAAAGAAA
CAATACAAGCATCACAGGAGATTAGACTATTGCCAAACCTTAATGGGAGA
CAGAAACTGGCGTTCATTCACAGTCTATTAGACGATCCCAGCCAATCCGC
CAATCTACTTGCAGAGGCCAAGAAACTGAACGATGCACAGGCACCGAAA

Seq. ID No: 261

>affibody_tag_3AACGCCGCAATTAGGTCATCCTCAGCATCTAGCGG
CGGGTCAGGCGGGTCATCTTCTTCAGGCGTGGATAACAAGTTTAACAAGG
AATTGGGCTGGGCGACATGGGAAATTTTTAACCTACCAAATCTGAATGGA
GTCCAGGTAAAGGCGTTTATCGATTCCCTGAGGGACGATCCCTCCCAGTC
CGCGAATCTACTAGCGGAGGCAAAGAAATTGAATGACGCTCAAGCGCCGA
AG

Seq. ID No: 262

>affibody_tag_4ATGGTGGACAACAAATTCAACAAGGAGCTGGGTTG
GGCCACCTGGGAAATCTTCAATCTTCCAAACCTTAACGGCGTACAAGTAA
AGGCTTTCATCGACTCTCTAAGAGACGATCCGTCACAGAGTGCTAATCTG
TTGGCCGAAGCGAAGAAGCTGAATGACGCTCAGGCTCCCAAAGGCAACGC
GGCCATAAGATCAAGCTCAGCTTCCTCTGGAGGTAGCGGCGGTTCCAGTT
CTTCA

Seq. ID No: 263

>affibody_tag_5ATGGTAGACAACAAGTTCAATAAAGAGATGAGAAA
TGCTTACTGGGAGATCGCTCTGCTACCAAACTTAAATAATCAACAAAAGA
GAGCATTTATTAGAAGTTTATATGACGACCCATCCCAGTCCGCGAACCTT
CTTGCGGAGGCAAAGAAATTGAACGATGCTCAGGCTCCGAAGTCAAGTAA
CGCAGCAATACGTTCTTCCAGTGCAAGCTCTGGGGGAAGTGGGGGAAGTT
CATCCTCC

Seq. ID No: 264

>affibody_tag_6AATGCCGCCATCAGGTCTTCATCAGCCTCAAGTGG
CGGCTCTGGAGGGTCCAGCTCCTCTGGGGTCGATAATAAGTTTAATAAAG
AGATGAGGAACGCATATTGGGAAATTGCCTTACTGCCGAATCTGAATAAT
CAGCAGAAAAGGGCTTTCATCAGATCCCTGTACGATGACCCGTCCCAGAG
CGCGAATCTTTTGGCAGAAGCGAAAAAGCTGAACGACGCACAGGCACCAA
AG

Seq. ID No: 265

>cofold_1ATGAAAATCGAGGAGGGGAAGCTAGTTATCTGGATCAACGG
GGATAAGGGCTATAATGGCCTAGCCGAAGTAGGAAAGAAGTTTGAGAAAG
ACACCGGTATAAAAGTGACAGTGGAGCACCCTGATAAGTTGGAGGAGAAG
TTCCCTCAGGTGGCTGCTACTGGGGATGGTCCAGATATTATCTTCTGGGC
TCATGACCGTTTCGGCGGATACGCCCAAAGTGGATTGCTTGCTGAAATAA
CGCCAGATAAAGCATTCCAGGACAAATTGTACCCATTCACCTGGGACGCG
GTGCGTTACAATGGAAAACTGATCGCTTACCCTATCGCGGTAGAAGCTCT
TTCACTTATATATAACAAAGATCTGTTGCCTAACCCTCCAAAGACCTGGG
AAGAGATTCCAGCATTAGACAAAGAATTGAAAGCAAAAGGCAAGAGTGCT
CTGATGTTTAATTTACAAGAGCCGTATTTTACCTGGCCCTTGATCGCCGC
GGACGGGGGTTACGCTTTTAAGTATGAAAACGGTAAGTACGACATCAAGG
ATGTTGGAGTAGATAACGCCGGAGCAAAAGCGGGTCTTACCTTTTTAGTA
GATCTGATCAAGAATAAGCATATGAACGCTGATACGGATTATAGCATCGC
AGAAGCGGCATTTAATAAGGGTGAGACAGCGATGACAATAAACGGCCCCT
GGGCATGGTCTAATATCGATACTTCTAAGGTGAATTACGGAGTGACTGTC
TTACCTACTTTCAAAGGGCAACCTAGCAAGCCGTTTGTTGGAGTCCTGTC
TGCGGGGATTAACGCAGCATCCCCTAATAAGGAGTTGGCTAAAGAATTTT
TGGAAAATTATCTGCTTACCGATGAGGGTTTGGAGGCGGTTAATAAAGAT
AAACCCCTGGGGGCGGTGGCTCTTAAATCATACGAAGAAGAGCTGGCGAA
AGATCCAAGAATAGCTGCTACAATGGAGAATGCACAGAAGGGTGAGATAA
TGCCGAATATTCCACAAATGTCTGCCTTTTGGTACGCTGTCAGGACTGCG
GTCATTAATGCTGCCTCAGGAAGGCAAACGGTGGACGAGGCGTTAAAAGA
CGCCCAGACAAACTCAAGTAGTAATAATAACAACAATAACAACAACAATA
ATTTAGGTATTGAAGGTAGG

Seq. ID No: 266

>cofold_2ATGGTGTCTAAGGGTGAGGAGTTGTTCACCGGAGTTGTCCC
AATTTTAGTGGAATTAGACGGAGACGTCAACGGACACAAATTCAGCGTCT
CAGGAGAGGGGGAGGGAGACGCGACTTACGGAAAACTTACCCTAAAACTG
ATATGTACGACGGGAAAACTTCCAGTACCCTGGCCTACTCTAGTTACAAC
TTTAGGGTATGGCCTACAGTGTTTCGCGCGTTATCCAGACCACATGAAAC
AACACGACTTTTTCAAGTCAGCTATGCCGGAGGGCTACGTTCAAGAACGT
ACCATTTTCTTCAAAGATGATGGAAATTACAAAACAAGAGCGGAGGTCAA
ATTTGAGGGAGATACCCTAGTAAATCGTATAGAACTGAAAGGGATAGACT
TTAAAGAAGATGGAAACATCCTAGGCCATAAACTTGAGTATAATTACAAT
TCCCACAATGTGTACATTACGGCTGATAAACAGAAAAACGGGATCAAGGC
CAATTTCAAGATTCGTCATAACATCGAGGATGGAGGGGTGCAATTAGCAG
ACCACTACCAACAGAATACTCCTATTGGTGACGGACCTGTCCTTTTACCG
GACAATCACTATTTGAGTTACCAGTCTGCTCTGTCCAAAGACCCGAATGA
GAAACGTGACCACATGGTGTTATTGGAGTTCGTGACAGCAGCTGGAATCA
CGCTTGGTATGGATGAGTTGTATAAA

Seq. ID No: 267

>cofold_3ATGGCGATGTTCTGCACGTTCTTTGAAAAACATCACCGTAA
ATGGGATATACTACTAGAGAAAAGTACCGGGGTCATGGAAGCGATGAAAG
TGACCAGCGAGGAGAAGGAGCAGCTTTCTACGGCGATTGATAGGATGAAT
GAAGGCCTGGACGCTTTCATTCAATTATATAATGAATCTGAAATAGATGA
GCCCTTAATCCAACTGGATGACGATACCGCCGAGTTAATGAAACAAGCTA
GGGATATGTATGGCCAGGAAAAGCTAAATGAGAAACTAAACACCATAATC
AAACAGATTTTATCTATATCCGTTTCAGAAGAAGGAGAGAAGGAAGGTTC
AGGCTCTGGA

Seq. ID No: 268

>cofold_4ATGTACCTGTTAGGAATCGGGCTGATTCTTGCACTAATTGC
CTGTAAACAAAACGTGTCCAGTTTAGATGAGAAAAATTCAGTGAGTGTTG
ACTTACCTGGAGAAATGAAGGTCCTAGTATCTAAGGAGAAAAATAAGGAT
GGAAAGTACGATTTGATAGCAACGGTTGATAAATTGGAACTAAAAGGTAC
TTCTGACAAAAACAATGGTAGTGGGGTTTTAGAAGGTGTGAAAGCGGACA
AAAGCAAGGTTAAACTGACGATCAGTGATGACGGATCAGGT

Seq. ID No: 269

>cofold_5ATGGCAGACAGGGATAGGTCTGGGATATACGGAGGGGCTCA
CGCAACCTATGGCCAGCAGCAGCAGCAAGGTGGAGGTGGTAGGCCGATGG
GGGAACAAGTCAAAGGAATGCTTCACGACAAAGGGCCGACGGCTAGTCAG
GCTCTGACGGTCGCGACACTTTTTCCCCTGGGCGGTTTATTGCTAGTGCT
TAGTGGACTGGCCCTAACGGCGTCAGTGGTGGGATTGGCTGTAGCTACCC
CAGTTTTCTTGATATTTAGTCCTGTTCTAGTACCAGCCGCCCTTTTGATT
GGAACAGCGGTGATGGGCTTCCTGACGAGCGGCGCTTTAGGCTTAGGAGG
CTTATCTAGTCTTACTTGTCTTGCAAACACGGCACGTCAGGCTTTTCAAA
GAACGCCTGATTACGTAGAGGAGGCTCATAGAAGAATGGCGGAGGCTGCC
GCGCATGCCGGACATAAAACGGCTCAAGCGGGCCAGGCTATTCAGGGCCG
TGCTCAGGAAGCTGGGGCCGGAGGTGGAGCGGGA

Seq. ID No: 270

>oxidase_1ATGAAGATCTTAATCTTGGGCATCTTTCTGTTTCTATGTA
GCACTCCAGCTTGGGCCAAGGAGAAGCACTATTACATCGGGATTATAGAG
ACGACGTGGGATTATGCCAGCGACCACGGCGAGAAGAAACTTATTAGCGT
GGACACTGAGCACTCTAACATCTATCTTCAGAATGGCCCAGATAGGATAG
GACGTTTATATAAAAAGGCATTATACCTTCAATATACTGACGAAACTTTC
AGAACGACGATTGAAAAGCCTGTGTGGCTAGGGTTCCTGGGGCCTATCAT
TAAGGCGGAGACGGGAGATAAGGTCTACGTACACCTAAAGAACCTTGCTA
GTCGTCCTTACACATTTCACTCTCATGGTATTACATACTACAAGGAGCAT
GAGGGTGCTATTTATCCAGACAACACCACAGATTTCCAAAGAGCCGATGA
CAAAGTTTACCCAGGGGAACAGTACACCTATATGCTACTGGCAACGGAGG
AACAAAGTCCCGGGGAGGGCGACGGGAACTGCGTAACAAGAATCTACCAT
AGCCATATCGACGCACCAAAGGATATAGCAAGCGGCCTAATCGGGCCTCT
GATTATTTGCAAGAAGGACTCCCTGGATAAAGAGAAAGAGAAGCATATTG
ATCGTGAGTTTGTGGTGATGTTCTCAGTAGTTGACGAAAATTTCAGCTGG
TATCTGGAGGACAACATTAAGACATATTGTAGCGAACCGGAGAAGGTAGA
TAAAGATAATGAAGATTTTCAAGAGTCTAATAGGATGTATAGTGTAAACG
GTTATACCTTTGGCTCACTTCCAGGGCTGAGTATGTGCGCAGAGGATAGA
GTTAAATGGTACCTGTTTGGCATGGGGAACGAAGTAGACGTACACGCAGC
ATTCTTTCATGGACAGGCGCTAACTAACAAAAACTATAGAATCGATACAA
TAAATCTATTCCCAGCTACGCTGTTTGACGCCTACATGGTTGCTCAAAAC
CCCGGAGAATGGATGCTATCTTGTCAGAATTTGAATCACCTTAAAGCCGG
TTTGCAAGCATTCTTTCAGGTACAAGAGTGTAATAAGTCCTCATCTAAAG
ATAACATTAGAGGAAAGCACGTCCGTCATTACTACATCGCGGCGGAGGAA
ATAATTTGGAATTATGCACCCAGCGGCATAGACATCTTTACTAAAGAGAA
CTTGACTGCCCCTGGGTCTGACAGTGCCGTGTTCTTCGAGCAAGGTACCA
CCCGTATTGGTGGATCTTATAAGAAACTTGTCTATAGGGAATATACCGAC
GCTAGTTTCACTAACCGTAAAGAAAGAGGCCCTGAGGAAGAGCACCTAGG
AATCTTAGGGCCGGTCATTTGGGCGGAAGTGGGGGACACAATACGTGTTA
CCTTCCATAATAAAGGTGCTTATCCATTGTCCATCGAGCCAATCGGCGTA
AGATTTAACAAGAATAACGAGGGGACTTATTACTCTCCTAACTATAACCC
TCAGTCAAGATCTGTGCCCCCAAGCGCCTCACACGTTGCTCCAACTGAAA
CTTTTACTTACGAATGGACGGTACCCAAAGAGGTCGGGCCCACTAATGCT
GACCCTGTATGCCTAGCTAAGATGTACTATTCCGCTGTAGATCCTACCAA
AGATATCTTTACGGGGTTAATAGGACCCATGAAAATCTGTAAGAAAGGTA
GCCTACACGCCAATGGCAGACAAAAGGACGTGGATAAGGAGTTTTATCTT
TTCCCTACAGTTTTTGACGAAAATGAAAGCCTTCTATTGGAGGATAATAT
CCGTATGTTCACTACCGCGCCCGACCAAGTTGATAAAGAGGACGAAGACT
TTCAAGAGAGTAATAAGATGCACAGTATGAACGGATTCATGTACGGCAAT
CAGCCGGGCCTTACCATGTGCAAGGGTGATTCAGTAGTGTGGTATCTATT
CAGCGCAGGCAACGAGGCAGACGTGCATGGAATTTATTTTTCAGGAAACA
CTTATCTTTGGAGAGGCGAAAGACGTGACACAGCAAATCTTTTCCCACAG
ACGAGTCTAACGCTACACATGTGGCCAGATACGGAAGGAACTTTCAATGT
GGAGTGTCTTACGACCGATCATTACACCGGGGGAATGAAGCAGAAGTACA
CCGTCAACCAGTGCAGGAGACAAAGCGAGGATTCAACCTTCTACCTTGGA
GAACGTACATACTACATTGCAGCTGTCGAGGTGGAGTGGGATTATTCTCC
TCAGCGTGAATGGGAAAAAGAACTTCATCACCTGCAAGAGCAAAATGTCT
CTAACGCGTTTCTCGACAAAGGTGAGTTCTACATAGGATCAAAGTATAAG
AAAGTGGTTTACCGTCAATATACAGACAGTACATTTAGAGTCCCCGTGGA
AAGAAAGGCAGAGGAAGAGCACCTGGGCATACTAGGCCCTCAGTTGCACG
CGGACGTCGGGGACAAAGTAAAGATCATATTTAAGAACATGGCTACTAGG
CCGTATAGCATACATGCACACGGAGTACAAACCGAGAGTTCCACAGTCAC
ACCAACGTTGCCGGGCGAAACTCTGACTTATGTGTGGAAAATTCCTGAAA
GATCAGGTGCTGGAACGGAAGACAGTGCTTGCATTCCCTGGGCGTACTAT
TCAACAGTCGATCAGGTCAAAGACCTTTACAGTGGTTTAATAGGGCCTCT
AATAGTCTGTAGAAGACCTTACCTTAAAGTCTTTAATCCTCGTAGGAAAC
TTGAGTTTGCGTTATTATTCCTGGTCTTCGACGAGAACGAAAGCTGGTAC
TTAGACGACAACATTAAAACGTACTCCGACCACCCAGAGAAAGTTAATAA
GGACGACGAGGAATTTATAGAATCTAACAAGATGCACGCTATCAACGGTA
GAATGTTTGGGAATCTTCAGGGTTTGACGATGCACGTGGGAGATGAGGTC
AACTGGTATTTGATGGGTATGGGAAACGAAATAGATCTGCATACCGTCCA
TTTTCATGGCCATAGTTTCCAGTACAAACATAGGGGAGTCTACTCAAGCG
ACGTATTTGATATTTTCCCGGGTACCTACCAAACTCTGGAAATGTTTCCC
CGTACGCCGGGTATTTGGCTGTTACATTGTCATGTAACGGATCACATTCA
TGCAGGGATGGAAACGACCTATACAGTATTGCAAAACGAGGATACAAAAA
GCGGA

Seq. ID No: 271

>oxidase_2ATGGGACTGAACTCCGCGATACCGTCTTTAGCGATTTTGG
CACTGTCCGTAGGATCTTATGCCGCAATCGGGCCAGTATCTGACCTACAC
ATTGTAAACAAAGATTTGGCACCCGACGGCGTGCAGAGGCCCACAGTATT
AGCTGGCGGCACGTTCCCCGGAACGTTAATAACTGGACAGAAGGGTGATA
ATTTTCAACTTAACGTTATCGATGACCTTACCGATGACCGTATGCTAACC
CCTACGAGCATCCATTGGCATGGCTTTTTCCAGAAAGGTACTGCCTGGGC
AGACGGTCCAGCTTTTGTTACGCAATGCCCGATTATTGCGGACAATAGTT
TCTTATACGATTTTGATGTGCCAGACCAGGCGGGAACTTTCTGGTACCAC
TCCCATTTATCAACACAATACTGCGACGGTCTACGTGGGGCATTTGTCGT
ATATGACCCGAACGATCCCCATAAAGATCTTTATGATGTCGATGATGAAA
GTACAGTCATCACACTAGCAGATTGGTACCACGTCTTAGCGCAAACAGTG
GTAGGCGCTGCTACGCCAGACAGCACATTGATTAATGGCCTAGGTAGGTC
CCAAACTGGTCCCGCAGACGCAGAGTTAGCCGTAATCAGCGTAGAGCACA
ATAAGAGGTATAGATTCAGACTTGTGTCTATATCTTGCGACCCAAACTTC
ACCTTCTCTATAGATGGTCACAACATGACCGTAATCGAGGTCGATGGGGT
AAATACCCGTCCTTTAACCGTGGATTCCATTCAAATTTTCGCCGGCCAAA
GATATTCTTTTGTCTTGAATGCAAATCAGCCGGACGACAACTACTGGATT
AGAGCCATGCCGAATATAGGTAGGAATACGACGACCCTAGACGGTAAGAA
CGCGGCTATACTAAGATATAAAAATGCTTCCGTAGAAGAGCCCAAGACAG
TTGGCGGTCCAGCTCAGAGCCCATTGAATGAAGCCGACCTTCGTCCGCTT
GTGCCAGCACCCGTGCCGGGAAATGCGGTCCCGGGAGGTGCAGATATTAA
TCACAGATTGAACCTGACATTTTCCAATGGGCTGTTCTCCATAAATAACG
CAAGTTTCACAAACCCATCCGTACCGGCATTACTGCAGATCCTGAGCGGT
GCACAAAACGCGCAGGACCTTCTGCCAACAGGTTCCTACATTGGGTTAGA
GCTGGGGAAGGTGGTAGAACTTGTCATCCCGCCACTAGCGGTAGGGGGTC
CTCATCCATTCCACTTACACGGGCATAACTTCTGGGTTGTCAGATCAGCC
GGGAGCGATGAATATAACTTCGACGATGCTATACTTAGGGATGTGGTTTC
CATTGGTGCGGGCACGGACGAGGTGACCATTAGGTTTGTCACCGACAACC
CGGGGCCCTGGTTCTTACACTGTCACATAGATTGGCATCTGGAAGCGGGA
CTAGCTATCGTATTCGCTGAAGGTATAAACCAGACTGCCGCTGCAAACCC
GACCCCTCAAGCATGGGACGAACTATGCCCTAAATACAACGGGCTTAGCG
CGAGCCAAAAGGTTAAACCGAAAAAGGGTACCGCTATT

Seq. ID No: 272

>oxidase_3ATGAGTAGGTTCCAGTCTCTGCTATCTTTCGTACTTGTAA
GTTTGGCGGCCGTTGCCAATGCTGCTATAGGCCCTGTTGCCGATTTAACT
TTGACAAATGCTGCAGTGAGTCCGGACGGATTTAGCAGAGAGGCTGTCGT
CGTTAATGGAATTACCCCAGCCCCTCTAATTGCTGGTCAGAAAGGCGATA
GATTCCAATTAAATGTCATTGATAACTTAACAAACCATACGATGCTAAAA
ACCACATCAATCCACTGGCATGGCTTCTTCCAGCACGGCACGAACTGGGC
CGATGGCGTGTCTTTCGTGAACCAATGCCCAATCGCCAGCGGCCACTCCT
TTTTGTACGATTTTCAAGTCCCGGACCAGGCAGGCACATTTTGGTATCAT
AGTCACCTGAGTACGCAGTACTGTGATGGTTTAAGGGGTCCGTTTGTGGT
CTACGACCCGAATGATCCCCAAGCGTCCTTATATGATATCGACAACGACG
ACACAGTAATCACGTTGGCAGATTGGTACCATGTTGCAGCCAAGTTGGGC
CCAAGGTTTCCTCTAGGAGCAGACGCCACCCTAATTAATGGACTAGGCCG
TTCACCCGGGACAACAACAGCCGACCTAGCCGTCATTAAAGTGACCCAAG
GCAAGAGATATAGATTTCGTTTAGTTTCTCTTAGCTGTGACCCTAACCAC
ACCTTTTCTATTGATGGCCATACGATGACTGTGATCGAAGCTGACAGTGT
AAACACCCAACCACTTGAAGTAGATTCAATTCAGATCTTTGCGGCTCAGC
GTTACAGTTTCGTTTTAGATGCGTCCCAACCAGTGGATAACTACTGGATT
AGGGCAAACCCAGCATTTGGGAATGTGGGTTTTGCAGGTGGAATTAACTC
TGCGATTCTGAGATACGATGGAGCCCCCGAAGTTGAGCCGACCACAACCC
AGACCACCTCCACCAAACCGCTTAATGAAGCTGACCTACATCCACTGACA
CCTATGCCCGTTCCGGGTAGACCTGAGGCCGGGGGCGTAGACAAGCCACT
TAATATGGTATTTAACTTTAATGGAACTAATTTCTTCATTAATAATCATT
CCTTCGTACCCCCGAGCGTACCCGTTCTGCTTCAGATTCTATCCGGCGCG
CAGGCTGCACAAGACCTTGTCCCAGACGGGAGCGTGTACGTCTTACCGTC
AAATTCAAGCATAGAAATATCTTTTCCAGCCACCGCCAATGCCCCGGGCA
CGCCCCATCCGTTTCACCTGCATGGCCACACTTTCGCCGTCGTCAGGTCT
GCCGGATCTAGTGAATATAACTATGACAACCCAATCTTCAGGGATGTTGT
CAGCACGGGTCAGCCAGGCGATAACGTGACTATAAGGTTCCAGACTAATA
ATCCCGGTCCCTGGTTTTTGCATTGCCATATAGACTTTCACCTAGAGGCC
GGATTTGCAGTTGTTCTTGCGGAGGATACCCCTGATACGGCAGCAGTGAA
TCCAGTGCCACAGTCCTGGAGCGACTTATGCCCAATCTATGACGCGCTAG
ACCCTTCTGACTTG

Seq. ID No: 273

>oxidase_4ATGAAATTCTTGTTACTATCCGCACTGCTTTTCCTGCATT
CTTCATTGGCTTGGACTAGGGAGAAGCACTACTACATCGGGATTACCGAG
GCGGTATGGGATTACGCGAGCGGAAGCGAAGAAAAAGAGTTAATTTCCGT
AGACACAGAACAATCCAATTTCTACCTTAGGAATGGCCCTGACAGGATCG
GTCGTAAATACAAGAAAGCCTTGTACAGCGAGTACACAGACGGCACATTT
ACAAAAACCATAGACAAACCAGCCTGGTTGGGATTCCTGGGCCCCGTAAT
AAAGGCCGAAGTAGGGGATAAGGTTTCCGTACACGTGAAGAACTTCGCCT
CACGTCCTTACACATTTCATGCTCATGGTGTTACTTATACCAAAGCAAAC
GAGGGTGCTATTTACCCTGACAACACAACAGACTTCCAGAGAGCAGACGA
TAAGTTATTTCCGGGTCAGCAATATCTATACGTTCTTCGTGCGAATGAAC
CAAGTCCTGGCGAAGGGGATTCCAATTGCGTGACGAGGATATACCACAGT
CATGTTGACGCTCCAAAGGACATCGCTTCTGGACTGATCGGCCCCTTGAT
TCTATGTAAGAAGGGCAGTTTGCACAAGGAAAAGGAAGAGAACATTGATC
AAGAGTTCGTGTTGATGTTTAGCGTGGTCGATGAGAACTTGTCATGGTAC
TTGGAAGACAACATCAAAACATTTTGTTCCGAACCAGAAAAGGTTGATAA
AGACAACGAGGATTTTCAGGAATCTAACAGGATGTATTCTATAAACGGTT
ACACATTCGGAAGTTTACCTGGACTAAGCATGTGCGCCGAAGACCGTGTA
AAATGGTATCTTTTTGGTATGGGAAACGAGGTGGATGTTCATAGCGAACT
GTTCCACGGACAAGCCCTTACCTCCAAGAATTACCATACAGACATAATTA
ACCTGTTTCCCGCCACATTAATTGACGTGTCAATGGTGGCACAGAACCCC
GGCGTATGGATGCTATCCTGCCAGAATCTTAACCACTTAAAAGCTGGATT
GCAGGCATTCTTCCAAGTCCGTGATTGTAACAAACCATCCCCTGACGATG
ACATCCAAGACCGTCATGTCAGGCACTATTACATAGCCGCGGAGGAAACG
ATCTGGGATTACGCGCCATCAGGGACAGATACGTTTACAGGCGAAAATTT
TACCTCCTTAGGATCAGATTCGAGAGTGTTTTTCGAACAAGGAGCTACTA
GAATCGGCGGATCGTACAAGAAGTTGGTCTACAGGGAGTATACGGATGAT
TCCTTTACTAACAGGAAGGAGCGTGGCCCAGATGAGGAACATTTGGGGAT
ATTAGGGCCGGTGATCTGGGCGGAAGTCGGCGATATCATTAGGGTTACCT
TTCACAATAAGGGTCAGTTTCCGCTAAGTATACAACCTATGGGAGTGCGT
TTCACTAAAGAAAATGAGGGAACTTATTATGGGCCAGATGGAAGGAGCAG
CAAACAAGCGAGCCATGTCGCCCCGAAAGAAACTTTCACTTACGAATGGA
CTGTACCTAAAGAAATGGGACCGACCTACGCCGATCCCGTTTGTTTGTCC
AAAATGTATTACAGTGGAGTAGATCTAACTAAAGATATATTTACAGGCCT
TATTGGCCCTATGAAGATATGCAAGAAAGGAAGTTTATTGGCTGACGGTA
GACAGAAAGACGTAGATAAAGAGTTCTACCTTTTCGCGACGGTCTTTGAC
GAAAATGAGAGTCTTCTACTGGACGACAATATTAGAATGTTTACTACCGC
GCCTGAAAACGTAGATAAGGAAGACGAGGACTTCCAGGAATCAAACAAAA
TGCATTCTATGAATGGCTTTATGTATGGCAACTTGCCCGGGCTAAACATG
TGCCTTGGTGAATCTATCGTTTGGTATCTTTTCTCTGCGGGCAATGAAGC
CGATGTTCATGGCATATACTTTTCAGGCAACACTTATTTGTCAAAAGGAG
AGAGACGTGACACGGCCAATCTGTTCCCTCACAAGTCCTTAACGCTTCTG
ATGACACCTGACACAGAGGGGAGCTTTGATGTCGAATGCCTGACAACTGA
CCATTATACAGGAGGCATGAAGCAAAAGTACACGGTCAACCAATGCAAAG
GACAATTCGAAGACGTCACCCTGTACCAGGGTGAACGTACGTACTACATA
GCGGCTGTAGAAGTAGAATGGGACTATAGTCCCAGCAGGGATTGGGAGAT
GGAGCTTCACCATCTGCAGGAGCAAAACGTGAGTAACGCATTCTTAGATA
AGGAAGAATTTTTCATCGGTTCAAAATATAAGAAAGTGGTCTACAGAGAA
TTTACAGACAGCACATTCAGAGAGCAGGTTAAACGTCGTGCGGAGGAAGA
GCACTTGGGTATGTTGGGGCCCTTAATCCACGCAGACGTAGGTGCTAAGG
TCAAGGTCGTCTTTAAGAATATGGCTACGAGGCCGTATTCAATACACGCG
CATGGGGTCAAAACCAAGAGTTCCACCGTAGCACCAACACTACCGGGTGA
AGTAAGAACCTACATATGGCAAATTCCCGAAAGAAGTGGTGCAGGTACCG
AGGATTCACCCTGCATCCCGTGGGCTTACTATAGCACGGTAGATCGTGTA
AAAGACTTATATAGCGGCCTGATAGGGCCGTTAATAGTATGCAGAAAATC
ATACGTTAAAGTCTTCAATCCTAAGAAGAAGATGGAATTTTCTCTGTTAT
TTCTGGTGTTCGATGAGAACGAGTCATGGTACCTTGATGATAATATTAAT
ACATATCCAGATCATCCTGAGAAGGATAATAAGGATAATGAAGAGTTTAT
TGAGAGTAATAAGATGCACGCAATCAATGGGAAAATGTTCGGAAACCTTC
AGGGGCTGACCATGCACGTAGGTGACGAAGTAAACTGGTACGTCATGGCT
ATGGGCAATGAGATCGATTTACACACAGTTCACTTTCATGGCCACTCTTT
TCAGTATAAGCACAGAGGGATTCATAGTAGTGATGTATTCGACTTCTTTC
CTGGAACGTATCAAACCCTAGAGATGTTCCCTCAAACGCCCGGAACGTGG
TTGCTACATTGCCACGTTACCGATCACATACACGCTGGTATGGTTACTAC
GTACACTGTGCTTCCAAACCAGGAAACCAAATCCGGG

Seq. ID No: 274

>oxidase_5ATGAACTTCGTCACGGCGCTGCCTCTGATTGCGCAGTTGA
TAGGAACAGCAAGGGCGGCTATAGGCCCGGTCACAAACCTTTTGGTGAAA
AACGCGGACATACCCCCGGACGGATTCACTAGGGCTGCGGTTGTCGCTAA
TAATCAGTTTCCTGGTCCTGTTATCAGGGCCACTAAAGGAGATACGCTTT
CCCTAAACGTGGTAAATCAGCTAACTGATGCCACCATGCTTATGGGGACT
TCAATACATTGGCACGGATTCCACCAGAAGGGTACCTCTTGGGCCGACGG
AGTTGTAGGCGTTACCCAATGTCCGATAGCTCCCGGACATAGCTTCTTGT
ATCAATTCCCTACAGCCAATCAAGCTGGGACTTTCTGGTACCACTCTCAT
TATTCCACACAATACTGCGATGGTCTAAGGGGAGCGTTAATAGTTTATGA
CCCAACTGACCCATACAGGACCTGGTATGACATTGACGACGAGTCTACCA
TAATTACACTAGCGGATTGGTACCATAAGGCTGCCCCTCTGCAAACGTTA
AGAACCGCGAAAGAGGATAGCGTACTGATCAACGGACAAGGCAGAGTCCC
AGGGGATAAGACCACCGACAGTACACCGTTGTCCGTGATTAATATAATTC
CTCAGAAGAGATATAGGTTTAGGCTGATTTCAATATCATGCGATCCAGCA
TTTTCTTTTTCAATAGACGGGCATAGCATGACTGTCATCGAGGCCGATTC
ACAAAGCGTCCAACCGTTGACGGTCAACGAGATAACTATTTTCGCGGGAC
AGCGTTATTCTTTCATCCTGTACGCGAACAACCCGGTTGGTAATTACTGG
ATTCGTTCACAGCCTACATACCCTGACGACGGTATTCAAGGGTATGCAGG
GGGTATCAACTCCGCAATTTTGAGGTATTCAGGCGCTCCTGCCGTCAACC
CAACTACGAAGAAGGCATCCATTACCATCCCGCTGGTAGAGGCCGATCTA
AGACCATTATATAGTCCCGCCGCTCCTGGATTGCCTTCACCCGGTGCGGC
AGATGTGAATATCAAACTGGATATAAGTTATAATTCTCCGTCTGAGACAT
TTTTCGTAAACAACTCCACATTTCCTGAAGTTCCTGTGCCTGTCCTTTTG
CAAATCCTGAGTGGCGCGCAAAGCGCCAACGATTTGTTGCCCGCTGGCAG
CGTGTATACCCTTCCGCCCAATAAAGTAATCGAGATTAGTATGCCAGGGG
GCCGTCCTGGGAGTCCGCATCCTATGCATCTACATGGTCATGACTTCAGT
GTGGTAAGATCTGCAGGATCAAATAGGTATAATTATGCCAACCCCGTTAG
GAGAGACGTAGTGAATATTGGGATGGAGGACACTGATAATGTCACGATCA
GGTTCCGTGTTTGCAGTCATACTTATTTGAGTTTACATTGTCATATCGAT
TTCCATTTGGAAGACGGACAGTCTGGGACCCTAGTTCCACCTCTTCCACA
CAGATTGCCGCCACGTGGAAGGATCAGGTGTAGACTACATAGAGGCATTT
TAGTAAGAGGCAGGCTTGGACCTGACCTTCAG

Seq. ID No: 275

>phosphatase_1ATGCAGGGGCCGTGGGTTCTTTTACTGTTGGGCCTG
AGACTACAACTATCTCTGGGCATTATCCCTGTAGAAGAGGAGAATCCAGA
TTTCTGGAACCGTCAGGCTGCCGAGGCCCTGGGTGCAGCTAAAAAGCTGC
AACCCGCCCAGACGGCAGCTAAGAATTTGATCATTTTCTTGGGAGACGGC
ATGGGTGTTTCTACTGTAACAGCGGCTAGGATTCTAAAGGGGCAAAAGAA
AGACAAACTTGGACCAGAAACGTTCCTTGCAATGGATCGTTTTCCTTATG
TGGCGCTGAGCAAGACTTACTCAGTGGACAAGCATGTCCCTGATTCCGGA
GCAACCGCAACAGCTTATCTTTGTGGAGTCAAAGGTAATTTTCAAACCAT
AGGGTTGAGTGCGGCAGCAAGATTTAATCAATGCAACACAACAAGGGGGA
ACGAAGTGATTAGTGTAATGAATAGGGCCAAGAAGGCTGGCAAGTCCGTA
GGGGTCGTTACGACCACTAGAGTTCAACACGCGAGCCCCGCTGGGGCGTA
TGCACACACTGTAAACAGAAATTGGTATAGCGACGCCGATGTGCCTGCTT
CTGCAAGACAAGAGGGATGTCAGGACATCGCTACGCAATTGATAAGTAAC
ATGGATATTGATGTTATTTTGGGCGGAGGCAGAAAATACATGTTCCCTAT
GGGAACGCCGGACCCTGAATATCCTGACGACTATTCACAAGGGGGAACTA
GGTTAGACGGAAAGAATTTGGTTCAGGAGTGGTTGGCCAAACACCAAGGC
GCACGTTACGTGTGGAATCGTACGGAGTTATTACAGGCATCTCTTGACCC
ATCTGTAACGCATCTGATGGGCCTTTTCGAGCCCGGTGACATGAAATACG
AGATCCATCGTGATTCTACTTTGGACCCAAGTCTTATGGAAATGACTGAA
GCAGCTTTATTACTACTATCACGTAATCCGAGGGGATTCTTTCTTTTTGT
TGAGGGAGGAAGAATCGACCACGGGCACCATGAGTCTCGTGCCTATAGGG
CGTTAACCGAGACCATTATGTTCGACGATGCAATCGAGCGTGCCGGGCAA
CTGACCTCTGAGGAAGATACTCTGTCCCTTGTAACCGCGGATCATTCTCA
CGTATTCTCATTTGGCGGATATCCTTTACGTGGCAGTTCTATCTTTGGGT
TAGCTCCAGGTAAGGCGCGTGATAGAAAAGCCTACACAGTGCTTTTGTAC
GGGAATGGCCCCGGTTATGTTTTGAAAGACGGCGCAAGACCAGACGTTAC
CGAATCCGAAAGCGGTAGTCCAGAGTATAGGCAGCAAAGCGCAGTTCCTT
TGGATGGGGAGACTCACGCAGGGGAAGATGTTGCTGTGTTCGCGCGTGGA
CCGCAGGCTCACCTTGTGCATGGCGTGCAGGAGCAAACCTTTATTGCCCA
TGTGATGGCTTTTGCAGCGTGTTTAGAGCCCTATACTGCCTGTGACTTGG
CTCCACGTGCGGGCACAACAGATGCTGCACACCCCGGCCCCTCTGTTGTA
CCGGCTCTGCTTCCGCTACTTGCTGGCACGTTACTGCTATTGGGAACAGC
TACCGCACCT

Seq. ID No: 276

>phosphatase_2ATGCAAGGCGCTTGTGTGCTGTTGCTTCTTGGCTTG
CACTTGCAACTATCACTTGGTCTAGTTCCTGTCGAAGAAGAAGACCCAGC
GTTCTGGAATAGGCAGGCCGCGCAAGCATTAGACGTCGCCAAGAAGCTGC
AACCTATCCAAACGGCCGCTAAAAACGTCATCCTGTTCCTTGGCGACGGG
ATGGGGGTCCCTACAGTCACAGCCACACGTATACTTAAAGGCCAAATGAA
TGGTAAATTGGGGCCGGAAACTCCACTGGCTATGGACCAGTTCCCCTACG
TCGCATTATCCAAGACGTACAATGTAGACCGTCAGGTACCCGACTCCGCA
GGAACCGCCACGGCTTATCTATGTGGTGTAAAAGGCAACTACAGAACGAT
TGGGGTCTCTGCTGCCGCAAGATACAATCAATGTAAAACAACTAGGGGTA
ATGAAGTCACGAGTGTCATGAACAGAGCTAAAAAGGCGGGGAAGAGCGTA
GGCGTGGTCACAACAACGAGAGTACAACATGCTTCTCCCGCGGGAGCCTA
CGCACACACGGTTAATAGGAACTGGTACTCTGATGCAGACTTGCCGGCCG
ACGCGCAAATGAACGGTTGTCAAGATATAGCAGCGCAGCTAGTAAACAAT
ATGGACATCGATGTAATATTAGGGGGTGGCAGGAAATATATGTTCCCAGT
CGGGACGCCAGATCCGGAATATCCCGACGACGCTAGTGTAAATGGCGTCA
GAAAGAGGAAGCAGAATTTAGTTCAAGCCTGGCAGGCTAAACATCAAGGC
GCTCAGTACGTATGGAATAGGACCGCACTGCTTCAAGCTGCAGATGATTC
CAGCGTGACACATCTGATGGGCCTGTTTGAACCCGCAGACATGAAGTACA
ATGTACAGCAGGACCATACTAAAGATCCAACTCTACAGGAAATGACTGAG
GTCGCATTGAGAGTCGTTAGTAGAAACCCCAGGGGCTTTTACCTATTTGT
CGAAGGCGGGAGAATCGATCACGGTCACCACGACGATAAAGCGTACATGG
CCTTAACTGAAGCCGGCATGTTCGACAATGCGATAGCCAAAGCCAACGAA
CTTACGAGTGAACTAGACACGTTAATTCTAGTAACTGCGGACCACAGTCA
CGTCTTTTCATTCGGCGGCTATACTCTAAGGGGCACATCCATATTTGGGC
TTGCACCGTCTAAGGCTCTCGATTCTAAGTCATACACCAGCATATTGTAC
GGCAACGGACCTGGCTATGCATTAGGGGGTGGATCACGTCCCGACGTCAA
TGATTCAACCAGTGAAGATCCGTCTTACCAGCAACAGGCCGCCGTGCCTC
AAGCCAGCGAGACACACGGTGGCGAGGACGTTGCTGTGTTTGCTAGGGGA
CCTCAGGCACATTTGGTACACGGCGTCGAAGAAGAAACCTTTGTAGCCCA
CATAATGGCGTTCGCCGGATGTGTTGAGCCTTATACCGATTGTAACCTGC
CAGCTCCGACAACGGCAACTTCTATTCCAGACGCGGCACATCTTGCAGCC
TCTCCCCCACCTCTTGCCTTGTTAGCAGGTGCTATGCTGTTACTGCTTGC
TCCTACGCTTTAT

Seq. ID No: 277

>phosphatase_3ATGCAGGGTCCTTGGGTATTATTGTTGCTTGGATTG
CGTCTGCAGCTATCCCTTGGTGTAATCCCAGCCGAAGAAGAAAACCCAGC
TTTCTGGAATAGGCAAGCAGCGGAGGCCCTGGACGCCGCCAAAAAGCTGC
AGCCTATTCAGAAGGTAGCCAAAAATTTGATATTGTTCTTGGGAGACGGT
CTGGGTGTACCAACCGTAACAGCTACCCGTATCCTAAAGGGTCAGAAGAA
TGGCAAGTTAGGACCTGAAACACCGTTAGCTATGGACAGATTCCCCTACC
TTGCCCTGAGCAAGACTTACAACGTGGATCGTCAGGTACCTGATTCCGCT
GCAACAGCAACCGCGTATCTTTGTGGAGTCAAGGCCAATTTTCAAACAAT
CGGACTATCTGCAGCCGCCAGATTTAACCAATGCAACACAACTAGAGGAA
ACGAGGTTATTTCTGTGATGAATCGTGCAAAGCAAGCTGGAAAGAGTGTC
GGGGTGGTAACAACGACGCGTGTTCAACATGCTTCTCCTGCGGGAACATA
TGCGCACACTGTTAACAGGAATTGGTACTCAGACGCGGATATGCCCGCGT
CAGCCAGACAGGAGGGCTGCCAAGACATCGCCACTCAATTAATATCCAAC
ATGGATATCGACGTCATTCTAGGAGGCGGTAGGAAGTATATGTTCCCTAT
GGGGACACCAGACCCTGAATACCCCGCTGACGCCTCTCAGAATGGCATAA
GACTTGACGGGAAAAATCTAGTACAAGAATGGTTAGCAAAGCATCAGGGC
GCGTGGTATGTCTGGAATAGAACGGAGTTGATGCAGGCAAGCCTGGACCA
GTCAGTTACGCACCTGATGGGGCTTTTTGAACCCGGCGATACAAAGTACG
AGATCCACCGCGATCCCACACTTGACCCTTCTTTAATGGAAATGACCGAA
GCGGCACTGCGTTTGCTGTCCAGAAACCCCAGAGGCTTTTACCTTTTTGT
GGAGGGTGGTAGAATAGATCACGGACACCACGAGGGCGTGGCATACCAAG
CTCTAACTGAGGCGGTAATGTTTGATGACGCAATTGAAAGGGCCGGACAA
CTGACCTCCGAAGAAGATACTTTGACCTTAGTCACCGCCGACCATTCCCA
TGTCTTTTCCTTTGGTGGTTATACCCTGAGAGGTTCCTCTATATTCGGAC
TAGCTCCCTCTAAGGCGCAAGACAGCAAAGCATATACGAGTATCCTGTAC
GGGAATGGACCGGGCTACGTATTTAACTCAGGTGTTAGGCCCGACGTAAA
TGAAAGTGAGTCCGGCTCCCCAGACTATCAGCAGCAGGCGGCTGTTCCAC
TTTCATCAGAAACCCACGGAGGTGAGGACGTAGCGGTGTTTGCCAGGGGC
CCCCAGGCTCATTTAGTTCACGGAGTTCAAGAGCAGTCATTCGTTGCTCA
CGTGATGGCATTTGCCGCATGTTTGGAGCCCTACACTGCCTGTGATTTAG
CTCCTCCCGCTTGCACAACAGACGCGGCACATCCTGTCGCAGCTTCCCTG
CCATTATTAGCTGGAACTTTACTTCTTCTAGGAGCGTCAGCCGCTCCT

Seq. ID No: 278

>phosphatase_4ATGCAAGGCGCCTGGGTATTACTTTTATTGGGTTTC
CGTTTGCAGCTGTCTCTGAGCGTAATCCCTGTAGAAGAAGAAAATCCGGC
GTTTTGGACGCAAAAAGCTGCTGACGCGTTGAACGTGGCGAAGAAGTTAC
AGCCAATTCAGACAAGTGCCAAGAATCTAATAATATTTTTAGGAGATGGT
ATGGGTGTTGCAACAGTAACGGCCACGAGGATCTTAAAGGGTCAGCTGGA
AGGGAATTTGGGTCCCGAAACACCGCTGGCGATGGACCACTTCCCGTATA
TGGCTCTTTCCAAGACTTACTCAGTTGATAGACAGGTACCGGATAGCGCA
TCTACAGCTACTGCTTATCTGTGCGGCGTAAAGACAAACTATAAGACGAT
TGGGGTTAGCGCAGCTGCGAGGTTCGATCAATGTAACACAACGTTTGGCA
ATGAGGTTCTTTCTGTAATGTACCGTGCAAAGAAGGCGGGTAAATCCGTG
GGCGTGGGTGATCATACGAGGGTGCAGCACGCGAGTCCGGCTGGCACATA
CGTGCACACCGTTACGTCTAATTGGTATGGGGATGCAGACATGCCAGCCC
TGCCGTTGCAAGAGGGTTGCAAGGACATAGCGACGCAGTTGATTAGTAAC
ATGGATATAAACGTAATACTTGGTGGGGGCAGAAAATATATGTTTCCGGC
TGGTACACCCGATCCTGAGTATCCAAATGATGTCAATGAAACAGGCACGC
GTCTTGATGGCAAGAATTTAGTTCAAGAGTGGCTATCCAAACACCAGGGT
AGCCAGTACGTTTGGAACAGGCAAGAGTTAATTCAAAAGTCCTTGGACCC
TAGTGTTACCTATTTGATGGGTCTATTTGAGCCAGTAGACACGAAGTTCG
AAATACAACGTGACCCTCTGATGGACCCCAGTTTGAAGGATATGACAGAG
GCTGCGCTTCACGTGCTTTCTAGGAACCCTAAAGGATTCTACCTATTCGT
AGAGGGTGGCCGTATAGATAGAGGGCACCACTTGGGTACCGCCTATCTGG
CACTAACTGAGGCCGTCATGTTTGATTCTGCGATAGAAAGAGCATCACTA
CAAGCGTCTGAGCAAGACACCCTAACTATCGTGACGGCAGACCACAGCCA
CGTCTTTTCCTTCGGGGGTTATACATTAAGGGGAACCTCCATCTTTGGGC
TTGCCCCGTTGAATGCTTTGGATGGAAAGCCCTACACATCTATTTTATAC
GGGAACGGGCCGGGGTATGTGGGGACAGGGGAAAGACCCAACGTTACCGA
CGCCGAAAGCCACGATCCTTCCTACCAACAACAAGCAGCGGTCCCTGTTA
AAAGTGAGACAACTGTGGGAAAGGATGTGGCAATCTTTGCGCGTGGACCT
CAGGCACACTTACTGCATGGCGTACAGGAGCAGAATTATATAGCACACGT
TATGGCTTTTGCTGGGTGCTTGGAACCCTACACGGATTGCGGCCTAGCTC
CACCTGCTGACGAGAATAGACCTACGACACCTGTACAGAACTCCACGACA
ACGACTACGACTACTACCACAACCACTACAACCACCACTACTACTAGAGT
CCAAAACTCAGCTTCCTCACTTGGGCCAGCAACCGCCCCTCTTGCTTGGC
ATTACTGGCCGCGTCGT

Seq. ID No: 279

>phosphatase_5ATGTCAGGATCTAGCGTTACAGGCGGGGGTGCAAGC
CTACCTGCGGAATTGTACAAGGGGTCCGCTGACTCAATCTTGCCAGCCAA
TTTCTCTTATGCTGTTACGGGTAGCGGTACGGGTAAAAATGCGTTCCTGA
CGAACAACTCTAGCTTGTTCGGTACAACCGGCACAGTTCATTACGCGGGG
TCCGATTCTGTTCTGTCCGGCAGCGAATTGACCACCTACAATTCAAACTA
CAATGGCACCTATGGTCCCTTAATCCAGATACCTAGTGTGGCGACTTCTG
TTACCGTGCCGTACCGTAAAGATGGTAACACTACGCTTAATTTGACATCA
GCACAATTGTGTGACGCATTTAGTGGAGCTAAGACAACTTGGGGTCAACT
ACTTGGCACAACTGACTCTACACCCATTAGGATAGTCTATAGGACAGGGT
CATCAGGGACTACCGAATTGTTTACTAGACACTTAAACTCTATTTGCCCC
ACAAGGTTTGCGACAAACAGCACCTTCACCAATGCGAGATTACCCGCCGG
AGGGACTCTACCTAGTAATTGGGTTGGTGTAGCAGCTACGTCTACAGTAG
TGAGCACGGTGAAAGCTACGAACGGCAGTCTGGGTTATGTATCTCCTGAC
GCAGTTAACATAAATTCCAATGCGGAGGTTAGTAGGGTCAATGGAAACTT
ACCTACACAGGCCAATGTGTCAACCGCGTTGGGGTCCGTGGCACCCCCGG
CCAATGCGGCAGATCGTGCAGATCCATCAAAGTGGGTCCCAGTATTCACA
AATCCTAGTGCCGGTTATTCAATCGTGGGATATACTAATTTCGTGTTTGG
CCAGTGCTATAAAGACGCCTCTGTCTCCACGGACGTTAGAGCCTTTATCA
ATAAGCACTATGGAGGCACAACCACCAATGCAGCGGTCGCGGCGCATGGC
TTCATTCCGTTGACACCAGCCTGGAAATCCGCTATAGTATCCGCCTTTTA
TACTGGTACGTCTGAAAATTTAGCGATAGGCAATACAAACGTATGTAACA
CTAAGGGAAGGCCC

Seq. ID No: 280

>phosphatase_6ATGAACTTCGTGACAGCCTTACCTCTTATCGCCCAA
TTGATAGGGACAGCTAGAGCGGCTATCGGACCGGTAACTAACCTACTGGT
CAAGAATGCGGACATTCCGCCCGATGGTTTTACACGTGCTGCGGTGGTTG
CTAACAATCAGTTTCCTGGTCCGCTAATCACGGGTAATCAGGGGATTAAT
TTTCAGATCAACGTAGTGGCCAGATTAAATAACGAGTGTAGTTCCCGTCC
TCTGTGGTATCATTGGCATGGTTTCTTCCAGAAGGGAACGAATTGGGCTG
ACGGTCCGGCATTTGTAAACCAATGTCCAATTTCTACTGGAAATTCATTT
CTTTATGACTTCACGGCTGCCGACCAGGCTGGTACGTTCTGGTACCATTC
CCACCTAAGTACCCAATATTGTGACGGTCTGAGAGGGCCGATGGTCGTAT
ATGATCCAAACGATCCCCACGCAAGTCTATATGACGTAGACGACGAAAGC
ACCGTAATAACCCTTAGTGACTGGTACCACACTGCCGCCCGTTTAGGCTC
TGCCTTTCCGCTAGGTCCGGACTCAGTGCTAATCAATGGTCTAGGTCGTT
TTGCAGGAGGGGATGGAAATGCTGAACTAGCGGTAATCCCAGTAACTCAG
GGCAAGCGTTATAGATTTCGTCTGATTAGCTTATCTTGTGACCCTAACTT
CGTCTTTAGCATCGATAAACACAACATGACCGTTATCGAGGCGGACGCCG
TCAGTCACGAGCCTGTGACGGTAGATAGCATACATATCTACGCGGGACAG
AGATACTCATTTGTTCTATCCGCTCATCGTGACATAGACAACTACTGGAT
ACGTGCTCTTCCTTCAGGCGGCACTGTAAATTTCGTCGGGGGCGTGAACT
CAGCCTTAATAAGATATGATGGTGCAGCGGAAGTTGAACCAGTTACCAAC
ACTACGATGAGTATTGCCCCACTTGTTGAAACTGACCTTGTACCACTGGA
CAGTCCAGCGGCTCCCGGCGAGGCCAGCATAGGAGGAGTGGACTATGCAC
TTAGTCTTGTTCCATCTTTCGTTAGTCGTACCTTGTTTTGCGTCCGTTCA
ATAGCAGATGACCTTAGGTTACGTACGGGAAGAACCATATTAAGCGGTTC
AACGGAGCTACCCTCCTCCCATCCCCCTTGCAGAGTATATACTTTGCCCT
CTAATGCTACGATAGAATTGTCCTTTCCAATCACCGCCACAAACGCGCCG
GGGGCTCCCCATCCCTTCCACTTGCATGGACACGTTTTTAGTGTTGTTAG
ATCAGCGGGGTCTAGTGAGTACAATTACGCAAACCCTCCACGTAGAGATG
TGGTCAATACAGGTACAGCCGGTGACAATGTAACTATTCGTTTTAGGGTA
TGTTCTCACACTTACTTGTCATTGCATTGTCATATTGACTTTCACCTGGA
GGACGGTCAAAGTGGGACGTTAGTACCACCTCTGCCTCACCGTCTGCCAC
CGAGGGGGAGGATTCGTTGTCGTTTACACAGAGGTATTTTAGTCAGAGGA
AGATTAGGTCCTGACCTACAA

Seq. ID No: 281

>scaffoldGGGGGCGCTAGTGCGTCCGGTGTGGATAATAAATTTAATAA
AGAGAGAGTCATTGCTATTGGTGAAATTATGAGATTACCGAATTTGAATA
GCTTGCAAGTAGTCGCCTTCATCAATTCCCTTCGTGACGATCCTTCTCAA
AGTGCCAACCTTCTGGCGGAGGCGAAGAAGTTAAATGACGCCCAGGCCCC
TAAAGGAGGGAGTTCTGCTTCCAGTGCGGGCGGATCATCAGTAGATAATA
AGTTTAACAAGGAGGCGCAAACCGCCGGGGTTGAAATAATGGAGTTGCCA
AACTTGAATACACGTCAGCTATTGGCTTTCATTCAAAGTTTACGTGATGA
TCCCTCTCAGAGCGCGAATTTATTAGCGGAAGCTAAGAAATTAAACGATG
CTCAGGCCCCAAAGACAAGTGGAGGCTCCAGCGCGTCATCTGCAGGTGGA
TCAAGCGTGGATAATAAGTTCAACAAAGAGAGGAGGATGGCGGCCTACGA
GATAATCGATCTGCCTAATCTAAATTGGTTTCAATTAGAGGCGTTCATCA
CGAGCCTAAGCGATGACCCGAGCCAAAGCGCAAATTTGCTTGCCGAAGCG
AAGAAACTTAACGATGCGCAGGCACCCAAGGGATCAGGCTCTAACGCCGC
AATTCGTTCTAGCGGGAGTGGGTCCGTAGATAACAAATTTAACAAGGAAA
GAGTAATTGCTATTGGAGAAATCATGAGGTTGCCTAACCTTAACTCCCTT
CAGGTAGTGGCATTTATAAACAGTTTACGTGATGACCCTTCACAGAGCGC
AAACCTACTTGCAGAAGCCAAGAAATTAAATGATGCACAGGCGCCCAAAG
GAGGGTCAAGTGCGTCCTCTGCAGGAGGGAGTAGTGTTGATAATAAATTT
AATAAAGAAGCCCAAACAGCGGGTGTTGAAATTATGGAACTACCGAACTT
AAACACCCGTCAATTACTGGCATTTATTCAGAGCCTGAGAGATGATCCAT
CTCAATCCGCTAACCTCCTAGCTGAGGCGAAGAAACTTAATGATGCCCAG
GCGCCAAAAACCTCAGGTTCAGGTTCAGCAAACGCTGCTATTCGTAGTGC
AGGATCTGGAAGCGTAGATAATAAATTCAACAAAGAACGTCGTATGGCTG
CTTATGAGATCATCGACTTACCTAATCTAAACTGGTTTCAACTTGAGGCT
TTCATCACTTCATTATCTGATGATCCATCCCAGAGCGCCAACCTACTAGC
GGAAGCGAAGAAGCTAAACGACGCTCAAGCTCCTAAAGGTTCAGGGGCGT
CTGGTTCTGGTGCGGGCGGCAGTTCCGTAGATAACAAATTTAACAAAGAA
AGAGTTATCGCCATAGGGGAAATTATGCGTCTGCCGAATCTGAATAGTCT
GCAAGTCGTCGCATTTATAAACTCTTTACGTGATGATCCCAGCCAATCTG
CGAATTTACTGGCAGAGGCTAAGAAGCTAAACGATGCGCAAGCCCCGAAG
GGCTCTGGCGCGTCTGGTAGTGGGGCAGGTGGGAGCAGCGTAGACAACAA
GTTTAACAAGGAGGCCCAGACTGCAGGAGTCGAAATCATGGAATTACCTA
ATTTGAACACCAGACAGCTGCTGGCGTTTATACAATCTCTTAGAGATGAT
CCTAGCCAGTCCGCTAATTTACTAGCCGAGGCCAAAAAGTTGAATGACGC
ACAGGCACCGAAGACAAGTGGTTCCGGGTCCGCTAACGCAGCGATAAGGT
CCGCGGGTTCCGGATCTGTGGACAATAAGTTTAATAAAGAGCGTAGGATG
GCCGCGTACGAAATAATCGATCTTCCTAATCTAAATTGGTTTCAGTTAGA
GGCCTTTATTACTAGCCTTTCCGATGACCCCAGTCAGTCAGCGAACCTAT
TAGCGGAGGCCAAAAAGCTGAACGACGCGCAGGCACCTAAG

Seq. ID No: 282

>sec_1ATGCAGCTATTAAGGTGTTTTTCTATTTTCTCAGTTATAGCCTC
CGTGTTGGCCCAAGAGCTAACTACCATCTGCGAGCAGATCCCATCTCCTA
CGCTAGAATCAACCCCATATAGTTTATCAACGACCACAATCTTGGCTAAT
GGCAAA

Seq. ID No: 283

>sec_2ATGCTATCTTTGAAAACGCTGCTGTGTACGCTGTTAACGGTTTC
ATCAGTTCTAGCCACACCCGTACCCGCTCGTGATCCATCCTCTATTCAAT
TTGTCCATGAAGAAAATAAGAAGCGTTATTATGATTACGATCATGGAAGC
CTTGGTGAA

Seq. ID No: 284

>sec_3ATGAAACTTCAGTCACTGCTGGTAAGCGCGGCAGTCCTGACCAG
CTTAACGGAGAACGTGAACGCATGGAGCCCAAATAACTCTTACGTTCCCG
CCAATGTCACTTGTGATGACGATATTAACCTTGTGAGGGAGGCAAGTGGA
TTAAGCGACAATGAGACTGAATGGTTGAAGAAGCGTGATGCGTACACCAA
AGAG

Seq. ID No: 285

>sec_4ATGGAGGGCGTGTCACTAGAAAAACGAGAGGCTGAGGCA

Seq. ID No: 286

>sec_5ATGAAAAAGACAGCGATTGCTATAGCGGTAGCCTTGGCGGGCTT
TGCCACAGTCGCTCAGGCC

Seq. ID No: 287

>vac_1ATGTTCTCTTTGAAAGCTCTACTACCCTTGGCTTTGTTGTTGGT
AAGCGCAAATCAAGTTGCAGCGAAGGTTCATAAGGCTAAAATATATAAAC
ACGAACTGTCA

Seq. ID No: 288

>vac_2ATGACGAAGAACTTTATTGTTACACTGAAAAAGAACACTCCAGA
TGTCGAGGCGAAAAAGTTCCTGGATAGTGTACATCACGCAGGGGGTTCCA
TAGTACATAAATTTGACATAATTAAGGGCTATACAATAAAAGTTCCAGAT
GTGTTGCACCTAAACAAACTAAAAGAGAAACATAATGACGTGATAGAGAA
CGTGGAAGAGGACAAGGAGGTCCACACCAAT

Seq. ID No: 289

>vac_3ATGGAAGAGCAGAGGGAAATCCTTGAGCAATTAAAGAAGACATT
ACAAATGCTAACTGTGGAGCCTAGCAAGAACAACCAAATCGCAAATGAGG
AGAAAGAGAAAAAGGAAAACGAGAACTCCTGGTGCATTCTTGAACACAAT
TATGAAGACATCGCGCAGGAGTTTATTGATTTTATCTATAAGAATCCCAC
CACCTACCATGTCGTTTCATTCTTCGCAGAATTACTTGACAAGCATAATT
TCAAGTACCTATCTGAGAAGAGCAACTGGCAGGACAGTATCGGCGAAGAT
GGCGGT

Seq. ID No: 290

>6xHISHHHHHH

Seq. ID No: 291

>AADC_1METVNKSCCSLATPHLPLMSPHLLHSHRDSPVLKIIHSIVLTV
QNNHSCLQAFLGIGTDNVILVKTNDRGKMIPEDLDHKIQKVKSEGSVPFL
VSTTCGTTVFGAFDPLEGIADVCERHSLWLHVDAAWGGSALLSSRHRHLL
KGIERADSVTWNPHKLLGVGLQCSAFLLRDTTQLLERCHAANATYLFQTD
KFYNLQYDTGDKSIQCGRRVDCLKLWLMWKALGSKGLETRVDRVLDHTRY
LVEEMKRREGFRLIMEPEFVNLCFWYVPPSLRNKENSPDFWTRLGKVAPV
IKERMMKKGSMMVGYQPHGNMVNFFRQIVVNPEVTKEDLDFFLDEIERLA
EDL

Seq. ID No: 292

>AADC_2MWGCGNGDCIHVLLLISHTSPPPLSPHLLHSHRDSPVLKIIHS
IVLTVQNNHSCLQGHVPFYVSATAGTTVYGAFDPFVKIADICQKHGLWML
HVDAAWGGGLLMSRKHRHKMNGIERADSVTWNPHKMMGVLLQCSAILLKE
KGILQGCNQMCAGYLFQQDKQYDISYDTGDKAIQCGRHVDIFKFWLMWKA
KGTVGFEQQINKCLELSEYLYSKICNREDFEMVFKGEVSLHRLEEGIK

Seq. ID No: 293

>AADC_3METVNKSCCSLATPHLPLMSPHLLHSHRDSPVLKIIHSIVLTV
QNNHSCLQGHVPFYVSATAGTTVYGAFDPFVKIADICQKHGLWMHVDAAW
GGGLLLSKKHRTKLSGIERANSVTWNPHKMMGVPLVKDNMDLLKRCHSAE
ASYLFQQDKFYDVRYDTGDKSIQCSRRADAFKFWMMWKALGTLGLEERVN
RALALSKYLAKEIKKRDGFELIWEPEYANICFWYIPPSLRKMENKGPEYW
QRLNQVAPTIKERMMKKGSMMIGYQPHRDKVNFFRHIIISPQVSREDMDF
VLDEIDLLGRDL

Seq. ID No: 294

>AADC_4MNASEFRRRGKEMVDYVANYMEGIEGRQVYPDVEPGYLRPLIP
AAAPQEPDTFEDIINDVEKIIMPGVTHWHSPYFFAYFPTASSYPAMLADM
LCGAIGCIGFSWAASPACTELETVMMDWLGKMLELPKAFLNEKAGEGGGV
IQGSASEATLVALLAARTKVIHRLQAASPELTQAAIMEKLVAYSSDQAHS
SVERAGLIGGVKLKAIPSDGNFAMRASALQEALERDKAAGLIPFFMVATL
GTTTCCSFDNLLEVGPICNKEDIWLHVDAAYAGSAFICPEFRHLLNGVEF
ADSFNFNPHKWLLVNFDCSAMWVKKRTDLTGAFRLDPTYLKHSHQDSGLI
TDYRHWQIPLGRRFRSLKMWFVFRMYGVKGLQAYIRKHVQLSHEFESLVR
QDPRFEICVEVILGLVCFRLKGSNKVNEALLQRINSAKKIHLVPCHLRDK
FVLRFAICSRTVESAHVQRAWEHIKELAADVLRAERE

Seq. ID No: 295

>AADC_5MASGYPGAGAQQPPAAPASGSGSPVSMPYYASELARAKHEDDM
KMPEHGIEPRHCLRRIEDYHLLDFSERLNTSSYVNVVFEPEEETVANMGL
KVNLADQTVYPESFRMHNDTVNMIAKLWNCPKPADFDEYGCYAGAGTVGS
TEACLLGGLALKFRWRKWYAAKHGMDQNKVRGVYPNLVITTMFQAAWEKL
FKYMDIEPRFVTPSWKTFTMDPSGLEKVVDDKTIGVVCIMGNHYGGQYDP
VWEVNDVLEKINKEKGLQVGIHVDGASGGFIAPFQEGLPAWDFRLKNVLS
ISASGHKFGNSCCGTGWIIWRERKGLSDTVAINVSYLGGSADSYTLNFSR
PAQGVYVQ

Seq. ID No: 296

>AADC_6MAGSIDVMLRDLNLDTVESLPADFDPTTVINDILPPVVNDAKL
VNGIEKPREIVLGRNVHTSSLEVTEPDADDEVTGEREAYMASVLARYRKS
LLERTKHHLGYPYNLDFDYGALSQLQHFSINNLGDPFIESNYGVHSRQFE
VGVLDWFARLWELERNEYWGYITNCGTEGNLHGILVGREVFPDGILYASS
ESHYSIFKAARMYRMDCEKVNTLISGEIDCEDFKAKLSLHKDKPAIINVN
IGTTVKGAVDDLDLVIKTLEESGFSHDRFYIHCDGALFGLMMPFVKLAPK
VSFKKPIGSVSVSGHKFVGCPMPCGVQITRLEHINALSRNVEYLASRDAT
IMGSRNGHAPLFLWYTLNRKGYRGFQKEVQKCLRNAHYLKGRLTEAGIGA
MLNELSSTVVFERPQDEEFTRKWQLACQGNIAHVVVMPNINIDKLDHFVN
ELVERRAVWYENGKLKSPCVASEIGNSNCLCALHK

Seq. ID No: 297

>AADC_7MVRAVEKPVQAIVNAAFRGKDAYHVFRTTVLAAVLLRLWRHLR
RVMAHEGLKAYFMSLVAPHLKKLPYVQNKLKKEMDKTMTKMRNTFRKEVT
DPRTSLPVDGIPEKQILELIQHRKELDTKEWTKGMTTGCVYHGGQDHYDF
VGQIFASWGFANPLHPTTFPSLRQMDSEVVQMVINMYHGDSECCGAFTTG
GTESILMAMKAYRDWGKAEKGITDPNIVICNTAHAAFDKAGKYFNIFVKH
ARTNSEMEIDLGHLRSLIDSNTVAIVGSACQFSHGTVDPIQEMAKIAMKR
RVGLHVDCCLGGFLVPFMEKAGFQLPPFDFRVKGVTSISCDPHKYGFAPK
GSSVVMFSNRHLRHYMYCFLTEWSGGIYATATMTGSRAGGPVAATWASMC
KFGEKGYIETTKQIVGATKKIAAGIAEIEGLRVVGRPDVCVVAFTCTEGS
GMNCYAVGDCMHQDFHWELQSCQNPACVHLALTLPTSRNADKFVADLRQA
VEAVRSDKDGKFASTAGMYGTAASLPAAFFEDGAAAYLDAMCEAIPAGDA
LLPEEPATKESPAAAGAPAQATGGA

Seq. ID No: 298

>AADC_8MNASEFRRRGKEMVDYVITNYLEQIELRQVYPSVEPGYLRPMI
PDSAPEEGETYEDIMKDIERVIMPGVTHWNSPYFFAYFPAATSYPAMLAD
MLCGSLGCIGFSWAASPACTELETVMLDWLGKTIGLPEQFLAGTNGEGGG
VIQGTASEATLMALLAARTKVTRRLQAENPDLSEAEIISRMVAYSSDQAH
SSVERAGLISGVRMKKIPSDENFTARGEALKKALEEDKAEGFIPVFLCAT
LGTTTSCAFDNLMELGPICNAENMWLHIDAAYAGSAFICPENRYLMKGVE
FADSFNFNPHKWLLVNFDCSAFWVKKRSDLICAFKIDPVYLQHDQQESGL
VTDYRHWQIPLGRRFRSLKLWFVLRMYGVKGLQAHIRKHIRLAQEFHEFV
KNDDRFEICAPVILGLVCFCLKGSNTLNKSLLQKINTLKKIHLVPSCLGD
KFILRFAVCARTLESNHIVFAWKHIEELATEVLKEGEKQ

Seq. ID No: 299

>AADC_9MQNCNQMHASYLFQQDKHYDLSYDTGDKALQCGRHVDIFKLWL
MWRAKGTTGFEVQIDKCLELAEYLYDKIKNREGYELVIEGKPQHTNVCFW
YIPPSLRHMEDNEERMARLVKVAPVIKARMMEYGTTMVSYQPLGDKVNFF
RMVISNPAATHQDIDFLIDEIERLGQDL

Seq. ID No: 300

>AADC_10MWGCGNGDCIHVLLLISHTSPPPLSPHLLHSHRDSPVLKIIH
SIVLTVQNNHSCLQGHVPFYVSATAGTTVYGAFDPFVKIADICQKHGLWM
HVDAAWGGGLLLSKKHRTKLSGIERANSVTWNPHKMMGVPLFQCSAFLLR
DTTQLLERCHAANATYLFQTDKFYNLQYDTGDKSIQCGRRVDCLKLWLMW
KALGSKGLERRVDRVLDHTRYLVEEMKNREGFRLIMEPEFVNLCFWYVPP
SLRNKENSPDFWTRLG

Seq. ID No: 301

>AADC_11MGSLGTNPTSFSAFPDDKAAFEPLNPEDVRAYLHKAVDFISD
YYTNVESMPVLPNVKPGYLQDELTASPPTHSAPFDVTMKELRTSVVPGMT
HWASPNFFAFFPSTNSAAAIAGDLIASAMNTVGFTWQASPAATEMEVLAL
DWLAQLLHLPTTFMNRTSTGRGTGGGVILGTTSEAMLVTLVAARDAALRR
SGSVGVSDIPRLAVYAADQTHSTFFKACRLAGFDPANIRSIPTGPETNYG
LDPAKLLEVMQADADAGLVPTYVCATVGTTSSNAVDPVGAVADVAAMFNA
WVHVDAAYAGSACICPEFRHHLDGVERVDSISMSPHKWLLTCLDCTCLYV
RDAHRLSDSLETNPEYLKNDVTDSGEVTDLKDMQVGVGRRFRGLKLWMVM
RTYGTAKLQEHIRSDVAMAKMFEDSVRADNRFEVVVPRNFALVCFRIKAR
GDMTEEDADEVNRLLMENLNKTGKAYLAHTVVGDRFVLRFAVGSSLQEER
HVRSAWDLIKKTTSSIMD

Seq. ID No: 302

>AADC_12MDPLKAVEMVDENTICVAAILGSTLTGEFENVKLLNELLTKK
NKDTGWDTPIHVDAASGGFIAPFLYPDLEWDFRLPLVKSINVSGHKYGLV
YPGVGWVVWRSKGDLPDELIFHINYLGSDQPTFTLNFSKGNNISTHAYKK
PKCRFYFLTNQILSRRYLLHRLKSGTSLNSLL

Seq. ID No: 303

>AADC_13MQPGYLSRMLPDSAPNHPESLEDIFNDISAKILPGVTHWQSP
NYFAYFPSNSSIAGFLGEMLSAGLNIVGFSWITSPAATELEMIVLDWLAK
LLKLPDDFLSGGTS

Seq. ID No: 304

>AADC_14MVVDYKDWQIPLGRRFRSLKLWMVLRLYGIENLQCYIRNHIK
LAQQFEVLVAQDLRFEIVSPRIFSLVCFRLLPSQNCKDHGNELNHHLLDT
VNSTGKVFLSHTVLSGKYILRFAVGAPLTEERHVTAAWKVLQDEASALLQ
SL

Seq. ID No: 305

>AADC_15MGSLDIKQESSPLMTNPLDSEEFRRQGYMVIDFLAEYYKNIQ
KFPVRSQVEPGYLRKRLPESAPYEPESIERILKDVHDDIVPGLTHWQSPN
YYAYFPSSGSTAGLLGETLAAGFNVVGFNWISSPASTELESIVMDWLAEM
LNLPKSFTFSGDGGGVMMGTTCEAILTTITAARDRILDRIGREHINKLVV
YGSDQTHCSFFKSAKIAGILPNNFRQVKTSRVNAFSMRPDALRAAIQADA
DAGLVPFFLCTTVGTTSTAAVDPVALLCEVTKDYGMWVHIDAAYAGNACI
CPEFRHMINGVENADSFSFNAHKWFLTTLDCCCLWVKDPSSLVRCLSTNP
EYLKNKATDTQQVVDYKDWQITLSRRFRSL

Seq. ID No: 306

>AADC_16MDGQMLKPMDAEQLREYGHQMVDFVADYYKTIESFPVLSQVQ
PGYLRELVPTNPPTHPESLQDVLDDVKAKILQGVTHWQSPGYFAYFPCNS
STAGFLGEMLSAGINIVGFSWMSCPAATELEVIVLDWLSKL

Seq. ID No: 307

>AADC_17MVLRLYGQEGLQSYIRNHIALAKQFEELVIQDSRFEMVTPRR
FSLVCFRLLPRSTDENQADKLNRALLDAVNLTGAIFISHTVLSGMYVLRL
AVGAPLTEERHVIAAWKVLQEKATALLEGNVAQEPNGHAQLSNGVVELDG
ALDNGVTTEQHGHVDDLPIKSK

Seq. ID No: 308

>AADC_18MSLSRHIDAERLIEQIKEHPHKKHSSAESRRGLHRSWRRDED
AADLPKYTLPKHGINSKAAYQLLHDETALDGNPLLNLASFVHTWMPEDAD
KLIMENINKNIVDMDEYPAASLIHNRCISMLADLWKAPKEGKVIGTATAG
SSEAIMLGGLALKKRWQEARKAAGKDYFHPNIVFGSNAQVALEKFARYFD
VETRLVPVKEENGFVMNPHDAIPYIDENTIGVIVILGSTYTGHFEDVKLM
SDLLDDLEKRTGLDVKIHVDGASGAFIAPFAYPHLKWSFDVPRVVSINTS
GHKFGLVYAGLGWVLWRDESFLHRDLVFELHYLGSTEYSFTLNFSKPAAP
VIAQMFNFLNLGFEGYKKIAYKDMRNARMLSRALESTTYFKVFSNIHVPR
NSDSAHVSSNNKDDPETYHAGLPVVAFRLSDEFNQNYPNVRQVWIQTLLR
TKGWIVPNYNAPLGAENIEILRIVVRETLSEDLIERLIVDIVAVTESLTT
EEGNVFAGITASGAVVKPDLDEARPDSSNFNDSGDGEAQGQTGYSRQC

Seq. ID No: 309

>AADC_19MALSKHVNTDKLIRDSRDKKSPKEKAHHTATAHQEATYSYGD
RYVTNPVPKYNIASKGISADAAYRLIHDELALDGSTVLNLASFVHTWMPP
QGEQLVHENIAKNLIDSDEYPATQIIHTRCVSILADLWHAPSAKQAVGTA
TTGSSEAIQLGGLAMKKMWQARMKAAGKNIHEPGPNIVMGANAQVALEKF
ARYFDVECRLVPVSVESKYRLDPKKAMDFVDENTIGIFIILGSTYTGHYE
PVKEMSDLLDEYEKRTGIYVPIHVDGASGGFVAPFVHPKMVWDFKLPRVV
SINTSGHKFGLSYVGVGWVVWRDKAHLPKDLIFELHYLGSVEYSFSLNFS
RPAAPILAQYFNLVHLGFEGYRSVGLADMKNARELSRALEKTGYYTVLSD
IHRAVGAKDPHGIDDADIEAYEPGLPVVAFRFSDNFKEKHPEIQQKWIQT
LLRAKGWIVPNYELPPSLEQIEILRVVVRENVTEVLIDKLIDDIVEITEQ
LADSSSSMHSLNNLGHIQRPKKHEHPESNLKEGEGSDYSGTYARPC

Seq. ID No: 310

>AADC_20MALNAVSAARGSARQYISTFLTLDNAKSGLFYYVLLVQAIKV
KRHLRARGISASLKELYTWISQQIIRLLLRLPATRKKVASQMDQAKLDIE
NRLVPKGANVTRHLSLPSEGKSLEWITQEMDKMDTELGGTSDAWRQGKLS
GAVYHGGDELAKIIVAAYSRYCVSNPLHPDVFPAVRKMEAEIVAMCLKMY
RGPEGAAGAMTSGGTESIVMSVKTHRDWARSVKGIKEPEMVVPVSAHAAF
DKAAAYLGIKLHSIPVDSYTRQVNIKHVKRAINSNTIMIVGSCIGFPDGN
QDDIEALGALAKKYNIGLHVDCCLGSFIVPFLEPAGLAKGDNKGRYKLTP
FDFTVDGVTAISCDTHKYGFAPKGTSVIMYRSAELRRFQYYVNPIWPGGV
YASPSLSGSRPGALIAGCWAVMQYMGTEGYLSSCRDIVIATRKIADAITD
DIPELYVLGNPPASVVAFGSRNPTVDPLEVGDGMRKRGWHLNGLSSPKSV
HIACTRLTLPVVDQFIADLKDCVREAKVAPSGKGTMVSVYGLGNSSAVGP
DMVSQLASAFLDALYKA

Seq. ID No: 311

>AADC_21MELKTAANEICNSNQMCQGENYSQNMLLRDGLIDLKNQIKEG
DEGLGHGFSDFSNVFASDLLPARNGEKYTEGFLLEVFNILFSYIRKTFDR
KSKVLDFHHPHQLLEGLEGFNLELSDQPEPLEQILGDCRDTLKYGVKTAH
PRYFNQLSSGLDMVGLAGEWLTGAANTNMFTYEIAPVFIIMEGLLIKKMH
ELVGWGELEADGIFSPGGTISNLYSVLVARYKFFPIVKLKGMAALPRIVL
FTSEHSHYSFQKASATLGIGIENVIAVKCDERGKMIPSDLDEKIMAQKEK

Seq. ID No: 312

>AADC_22MWKAKGTRGFELQIDSCLENAEYLYKKLKSRNGFELVFPDEP
EHTNVCFWYIPPSLKGMPRDKEWNTKLHKVAAKIKAQMMEEGTVMVSYQP
LKNKPNFFRMVFSNPASKKSDIDFLLDEIERLGSDLEF

Seq. ID No: 313

>AADC_23MFGSQHQMDVAALDRQLKEDKESGKLPLLLVANAGTPGAGHT
DKLARLKELCHQYNIWLHVEGVNLATLALGYVSASVLAATKCDSMTLTLG
PWLGLPAVPAVTLYRHEDPSLSLAAGLTTSQPVEKLRALPLWLSLQYLGH
NGIVERIKHASQLSQRLLENLKDVTSIKTSVEPDGNSPVVVFKFFYDGPG
SGSTINLNTIERESDAMNQWLGEQLAALIPSCAVDTVELEDEGVCVRFNP
MMTSAVLGTTIEDVDQLVECVKVKIPIIHNTLQLKEEFRLEVERIAGLTY
VVDYSWAGLGVLRYDHVSEELDGSRREAELEKINASLLKKLNELESDLSF
SSGPEFGAEKNCVYIGMATEDVDVSELVETIAVMGREIEENSKLLENMTE
VVRKGILEAEVQLQKANEERLLEEGVLRQIPLVGSVLNWLSPVQATPKGR
TFNLTAGSLESTEITYASKAQANGTSPPPTPSLGHAKRHPGQKLFKRLSR
NSDAMSETSSVSHLEEVENLEASPTPEPQPGHPTEPPVPSVESNSEEPHE
AEALDTKTVESESLR

Seq. ID No: 314

>ADK1MSSSESIRMVLIGPPGAGKGTQAPNLQERFHAAHLATGDMLRSQI
AKGTQLGLEAKKIMDQGGLVSDDIMVNMIKDELTNNPACKNGFILDGFPR
TIPQAEKLDQMLKEQGTPLEKAIELKVDDELLVARITGRLIHPASGRSYH
KIFNPPKEDMKDDVTGEALVQRSDDNADALKKRLAAYHAQTEPIVDFYKK
TGIWAGVDASQPPATVWADILNKLGKD

Seq. ID No: 315

>AOQS_1MSNAAIRSSRAVSVSSSTKYYDFTVIGSGVAGLRYALEVAKQG
TVAVITKDEPHESNTNYAQGGVSAVLCPLDSVESHMRDTMVAGAHLCDEE
TVRVVCTEGPERIRELIAMGASFDHGEDGNLHLAREGGHSHCRIVHAADM
TGREIERALLEAVLNDPNISVFKHHFAIDLLTSQDGLNTVCHGVDTLNIK
TNEVVRFISKVTLLASGGAGHIYPSTTNPLVATGDGMAMAHRAQAVISNM
EFVQFHPTALADEGLPIKLQTARENAFLITEAVRGDGGILYNLGMERFMP
VYDERAELAPRDVVARSIDDQLKKRNEKYVLLDISHKPREKILAHFPNIA
SECLKHGLDITRQPIPVVPAAHYMCGGVRAGLQGETNVLGLFVAGEVACT
GLHGANRLASNSLLEALVFARRAVQPSTELMKRTRLDVCASEKWTRPVVA
TARLLGDEVIAKIIALTKEVRRELQEVMWKYVGIVRSTIRLTTAERKIAE
LEAKWETFLFEHGWEQTVVALEACEMRNLFCCAKLVVSSALARHESRGLH
YMTDFPFVEESKRIPTIILPSSPTTASWSSRRLQNISSSSLIDCGSGEGR
GSLLTCGDVEENPGPSSSSSSQTTELVPYKLQRLVKEFKSLTEPIDRLKW
VLHYASLLPQMPESSKTESNRVMGCTARVWLDAELGQDGKMRFCADSDSD
VSKGMCSCLIQVLDEASPVEVMELKTEDLAELNVGLLGGERSRVNTWYNV
LVSMQKKTRRLVAEREGKVPSFEPFPSLVLTAHGIEAKGSFAQAQAKYLF
PEESRVEELVNVLKEKKIGVVAHFYMDPEVQGVLTAAQKHWPHISISDSL
VMADSAVTMAKAGCQFITVLGVDFMSENVRAILDQAGFEKVGVYRMSDET
IGCSLADAASAPAYLNYLEAASRSPPSLHVVYINTSLETKAFAHELVPTI
TCTSSNVVQTILQAFAQMPELTVWYGPDSYMGANIVKLFQQMTLMTNEEI
ANIHPKHSLDSIKSLLPRLHYFQEGTCIVHHLFGHEVVERIKYMYCDAFL
TAHLEVPGEMFSLAMEAKKREMGVVGSTQNILDFIKQKVQEAVDRNVDDH
LQFVLGTESGMVTSIVAVIRSLLGSSANSKLKVEVVFPVSSDSMTKTSSD
SSNSIKVGDVALPVVPGVAGGEGCSIHGGCASCPYMKMNSLSSLLKVCHK
LPDLENVYGGFIAERFKRQTPQGKLIADVGCEPILHMRHFQANKELPDKL
VHQVLSCESKR

Seq. ID No: 316

>AOQS_2MNTLPEHSCDVLIIGSGAAGLSLALRLADQHQVIVLSKGPVTE
GSTFYAQGGIAAVFDETDSIDSHVEDTLIAGAGICDRHAVEFVASNARSC
VQWLIDQGVLFDTHIQPNGEESYHLTREGGHSHRRILHAADATGREVETT
LVSKALNHPNIRVLERSNAVDLIVSDKIGLPGTRRVVGAWVWNRNKETVE
TCHAKAVVLATGGASKVYQYTTNPDISSGDGIAMAWRAGCRVANLEFNQF
HPTALYHPQARNFLLTEALRGEGAYLKRPDGTRFMPDFDERGELAPRDIV
ARAIDHEMKRLGADCMFLDISHKPADFIRQHFPMIYEKLLGLGIDLTQEP
VPIVPAAHYTCGGVMVDDHGRTDVEGLYAIGEVSYTGLHGANRMASNSLL
ECLVYGWSAAEDITRRMPYAHDISTLPPWDESRVENPDERVVIQHNWHEL
RLFMWDYVGIVRTTKRLERALRRITMLQQEIDEYYAHFRVSNNLLELRNL
VQVAELIVRCAMMRKESRGLHFTLDYPELLTHSGPSILSPGNHYINRGSG
EGRGSLLTCGDVEENPGPGSVMFDPDTAIYPFPPKPTPLSIDEKAYYREK
IKRLLKERNAVMVAHYYTDPEIQQLAEETGGCISDSLEMARFGAKHPAST
LLVAGVRFMGETAKILSPEKTILMPTLQAECSLDLGCPVEEFNAFCDAHP
DRTVVVYANTSAAVKARADWVVTSSIAVELIDHLDSLGEKIIWAPDKHLG
RYVQKQTGGDILCWQGACIVHDEFKTQALTRLQEEYPDAAILVHPESPQA
IVDMADAVGSTSQLIAAAKTLPHQRLIVATDRGIFYKMQQAVPDKELLEA
PTAGEGATCRSCAHCPWMAMNGLQAIAEALEQEGSNHEVHVDERLRERAL
VPLNRMLDFAATLRG

Seq. ID No: 317

>ATMT_1MTPAAGKTFNTSIAGADDLIRLHLSESPHGASKAALQAAEREL
ARVNVYPDPERQELVRALAAHWGVGPEHIAVANGSDELVLATALTLGDRN
LPGLVTDGTFPGYRACLELLGRGCTAVPPDGTAVDVAGFAARLPGHGIGY
LCNPHNPSGAALTRQELAALVEVSGRSGVPLVFDEAYMEFAGPDVPQTRD
LTAAGDAPVVALRTFSKAYGLAALRVGYAVGRPDLIAGLRGTLRALPFSV
NRLAQAAAIAALGDPDFVDGVRRSTAERRRWFVGELDRRGRAHLPSVTNF
VAVAARDCARAQDRLAADFGILVRNAGLFGFPGYLRTSLGEKKDLERFLD
ALDEIEQNPGGGSGEGRGSLLTCGDVEENPGPMTAPLSRDGLRAMGESVF
RPAEWQGAAHTPLDADTAFNGFISTHVVFALEQLGLFAWFDESDRLDVPQ
YCWRRKLDERVFRQLVSAAEAFGYLDVHDDLVTPTPAWSELRRKIGFFTW
GVGGYHDVFANAASIARGERAFGKDVLRDEAMVALGSAQADMALMRDLLD
EQIAALDFSVIADLGSGISERVCRLVKSRPGARGLGVDISASATALAAGT
VERHELADRVQPICADVLDVLFHGRRIEGADQVDVAMSFMFLHDLLVDPT
TRTDVIPALRKAFPRAHTFLLADTTVRPRDEKDTLPVFSSGFELAHALMG
VPIYTREEYENLFHEGGLHLRRTVPFGAPHTYLFVLEAQ

Seq. ID No: 318

>ATMT_2MQALPVKGDTVSRPPTVHSLHHEHERADGMLRLHCNENPYGPP
SGVIASVTKELEGRCSTYPDSEVTALREALAGQVGVGTDMVAVGNGADEL
VLLITLASAGPGDTVVVTESTFPGYAASAAVAGATVRGVPLHRDRVSATA
LVEAVDDGARLVFVCNPHNPTGTVLSPAAVEEILRACERTGAVPVFDEAY
IEFAGPGFDHALDAVRAGRRLLVLRTFSKAWGLAALRAGYAVGPADLVAG
IMEARRPLPFSVNRLAQQAALAALGSPDHIAEVYERTTRERERLCRALTG
LGVAYVPSVTNFVMVKTPGNSTRFASRLADEHGILVRDLAPFGYPGHVRV
SVGTAEDTDQFCAALGSLLASPRSHAATGHGLGASSGAGGAGNAAIRSAR
DVLPVPTLDPVAPQDLFNGYVGAHAVFALTRLGVWDRLAEGSEPTVDALA
VQAGTDATGLMPLLRVAALLGYVSLTDGSAPAVRLTESGRELVRMRGFFT
WGVGGYHEVLRSLPALARGTSVFEQDVDRDGGMVAVGSGEVGREMMLPLE
QEVLATVDFRTVADLGCGDATRLLRLCDGHPHRRGTGIEINQGACVQANK
RVADAGLADRVDIVHGDALDLSGRTFPEVDLVTSFLMMHDLFDATGDPVG
VMRTLREVFPRARHFLIGDTVAQDWEERREGLPMFSVGFELVHAFMDTPI
MNRGTYEDAFAGAGLRVARREPLGAPSTWLWLLSTE

Seq. ID No: 319

>ATMT_3MRRRWAVTASASWWGTACELHASASAAYTPPCHSPGTGGRGTE
SGPMTAPVRQETRNYNASVPSADDLVRLHLSESPYGASPAAVAAVTGELE
RINRYPAPGREGLVQALARHWELPEEHIAVANGSDELVLATALTLGDPGS
PGLVTAGTFPGYLAALERIGRGAVQVPLAGSGTDTAAFADRLPGCGIGYV
CNPHNPCGSALTHDELHRLVAAARDSGTPLVFDEAYHEFGPPAQPQARTH
LREDTPVLALRTFSKAYGLAALRIGYALGPADLIAEVRRTLTVLPFSVNR
AAQAAALAALDDQEFLGSVRRDSAARRQWFCAELERRGYRYLPSVTNFVA
VEVAASAEAQDVLARDHGILVRDTGMFGFPGHLRVSLGSVEELRGFLDAL
DRVTAGSRGGGSGEGRGSLLTCGDVEENPGPMTGPVSTSAPSRWPRTWRP
NRLEPTSRGGQPGHAARRSPAAGRRRRRASEARPPPSGRQPAVRTERCER
VSPLNTLPSEWQGQAPTPLNPDTAFNGYICANVLHGLERLGVFELLRDEK
SLDMDRFCETNGLDSAVFRALVGAAESFGYLDVRGAQVRATSVGEDVARY
LGFFTWGVGGYHDIFASAAPVARGERRFGVDLHRDEGMVALGSAQADTAL
MRHILDEEIAGIDFRTLVDLGAGVSERVSRLVKARPGTRGIGIDISRPAT
ELARDTVAGYGLAGTVEPVCADVLDILFNGQEIDGGDAADVVMSFMFLHD
LLAAPERREEVVPRLRKAFPRAHTFLLADTTIRPRNEEGDGRLPVFSSGF
ELAHALMGVPLHTREEYEELFERGGMKLRRSVPFGAPHTYLFVLEAS

Seq. ID No: 320

>ATMT_4MTNDPSPRDARDELPVRDELRGQSPYGAPQLDVPVRLNTNENP
YPLPEALVERIAERVREAARSLNRYPDRDAVELRTELARYLTRTAGHEVT
AAHVWAANGSNEVLQQLLQTFGGPGRTAIGFEPSYSMHALISRSTGTGWI
SGPRNDDFTIDVDAARAAIAEHRPEVVFITSPNNPTGTAVRAETVLALYE
AAQAARPSIVVVDEAYGEFSHHPSLLPLIEGRRHLVLSRTMSKAFGAAGL
RLGYLAADPAVVDAVQLVRLPYHLSSVTQATALAALEHTDTLLGYVAQLK
GERDRLVAELRAIGYEVTESDANFVQFGRFDDSHAVWRQILDRGVLVRDN
GVPGWLRVTAGTPEENDAFLDAVRELKKEHDAGGGSGEGRGSLLTCGDVE
ENPGPSSSSSTRTDFAQSAVASIFTGAIASHAAVLADDLGLFDALAKGKL
RNRDLDRSPWLRNRIRISGALEALCRVGAVQRCTDGYELTDVGTELAGQV
PVFRLWLGGYASVLAGQISIGADPATGVHGGIVAESSGAIGARYLDETIV
NLLESLRPEGRICDIGCGTGARLLRVCRRVNQPGIGYDLSAKAVEAARET
VDEARRIGVDIDVRQGDATALTQDHPDVDIVTQAFMTHHIAPDEYCAAVL
RSYRSRFPRARYLVIFDTVPSQDSEEPEIFAPGFDYIHALQNMEPRSRGA
ARRMFTEAGYICREEVELAVPNSYAWVLEMRDREGPAS

Seq. ID No: 321

>ATMT_5MTNDPSPRDARDELPVRDELRGQSPYGAPQLDVPVRLNTNENP
YPLPEALVERIAERVREAARSLNRYPDRDAVELRTELARYLTRTAGHEVT
AAHVWAANGSNEVLQQLLQTFGGPGRTAIGFEPSYSMHALISRSTGTGWI
SGPRNDDFTIDVDAARAAIAEHRPEVVFITSPNNPTGTAVRAETVLALYE
AAQAARPSIVVVDEAYGEFSHHPSLLPLIEGRRHLVLSRTMSKAFGAAGL
RLGYLAADPAVVDAVQLVRLPYHLSSVTQATALAALEHTDTLLGYVAQLK
GERDRLVAELRAIGYEVTESDANFVQFGRFDDSHAVWRQILDRGVLVRDN
GVPGWLRVTAGTPEENDAFLDAVRELKKEHDAGGGSGEGRGSLLTCGDVE
ENPGPAQAAPTTVTEVFNHAITASAISAAWEMGAFDALRVSERLDADEFA
AREGLDTRSTHELFRALAAADIVSRDGAQIRRGPNFAEADRCKSLFHWMT
RGCGELFSTLPALVREKNRVGSFYRRDAAAISVACREINAEWWDPVFWPV
VSGLDFTSVADLGCGSGERLIRLARTGPEVMALGIDFAAGAIEVATAAVA
EAGLSDRISLVQGDATALEPRPEFAGVDLLTCFMMGHDFWPRAEAVASLR
RIREVFPDLKHFLLADATRTTSYPDTDMPVFSMAFELAHAVMGDYLPTLE
EWRPVFEEAGWRCEGEHPISVPADSVMFHLVPN

Seq. ID No: 322

>ATMT_6MTNDPSPRDARDELPVRDELRGQSPYGAPQLDVPVRLNTNENP
YPLPEALVERIAERVREAARSLNRYPDRDAVELRTELARYLTRTAGHEVT
AAHVWAANGSNEVLQQLLQTFGGPGRTAIGFEPSYSMHALISRSTGTGWI
SGPRNDDFTIDVDAARAAIAEHRPEVVFITSPNNPTGTAVRAETVLALYE
AAQAARPSIVVVDEAYGEFSHHPSLLPLIEGRRHLVLSRTMSKAFGAAGL
RLGYLAADPAVVDAVQLVRLPYHLSSVTQATALAALEHTDTLLGYVAQLK
GERDRLVAELRAIGYEVTESDANFVQFGRFDDSHAVWRQILDRGVLVRDN
GVPGWLRVTAGTPEENDAFLDAVRELKKEHDAGGGSGEGRGSLLTCGDVE
ENPGPSTEVSEAQARRAVADIFNSTLASSAIGAAWELGALDELRENGKLD
VSDFAVRHDLHEPAVVGMFTALASVGIVRREGATVVVGPYFDEANHHRSL
FHWLNQGSGELFRRMPQVLPNENRTGKFYQRDAGAISYACREISERYFDP
AFWAAVDGLGYTPTTVADLGSGSGERLIQIARRFPGVRGLGVDIADGAIA
MAEKEVAAKGFGDQISFVRGDARTIDQVSARGEFAEVDLLTCFMMGHDFW
PRENCVQTLRKLRAAFPNVRRFLLGDATRTVGIPDRELPVFTLGFEFGHD
MMGVYLPTLDEWDGVFEEGGWRCVKKHAIDSLSVSVVFELE

Seq. ID No: 323

>BH4reg_1MAASGEARRVLVYGGRGALGSRCVQAFRARNWWVASIDVVE
NEEASASVIVKMTDSFTEQADQVTAEVGKLLGDQKVDAILCVAGGWAGGN
AKSKSLFKNCDLMWKQSIWTSTISSHLATKHLKEGGLLTLAGAKAALDGT
PGMIGYGMAKGAVHQLCQSLAGKNSGMPSGAAAIAVLPVTLDTPMNRKSM
PEADFSSWTPLEFLVETFHDWITGNKRPNSGSLIQVVTTDGKTELTPAYF

Seq. ID No: 324

>BH4reg_2MTALTQAHCEACRADAPHVSDEELPVLLRQIPDWNIEVRDG
IMQLEKVYLFKNFKHALAFTNAVGEISEAEGHHPGLLTEWGKVTVTWWSH
SIKGLHRNDFIMAARTDEVAKTAEGRK

Seq. ID No: 325

>BH4syn_1MEGGRLGCAVCVLTGASRGFGRALAPQLAGLLSPGSVLLLS
ARSDSMLRQLKEELCTQQPGLQVVLAAADLGTESGVQQLLSAVRELPRPE
RLQRLLLINNAGTLGDVSKGFLNINDLAEVNNYWALNLTSMLCLTTGTLN
AFSNSPGLSKTVVNISSLCALQPFKGWGLYCAGKAARDMLYQVLAVEEPS
VRVLSYAPGPLDTNMQQLARETSMDPELRSRLQKLNSEGELVDCGTSAQK
LLSLLQRDTFQSGAHVDFYDI

Seq. ID No: 326

>BH4syn_2MHSPSLSAEENLKVFGKCNNPNGHGHNYKVVVTIHGEEAIM
KPLDHKNLDLDVPYFADVVSTTENVAVYIWENLQRLLPVGALYKVKVYET
DNNIVVYKGE

Seq. ID No: 327

>BH4syn_3MPSLSKEAALVHEALVARGLETPLRPPVHEMDNETRKSLIA
GHMTEIMQLLNLDLADDSLMETPHRIAKMYVDEIFSGLDYANFPKITLIE
NKMKVDEMVTVRDITLTSTCEHHFVTIDGKATVAYIPKDSVIGLSKINRI
VQFFAQRPQVQERLTQQILIALQTLLGTNNVAVSIDAVHYCVKARGIRDA
TSATTTTSLGGLFKSSQNTRHEFLRAVRHHN

Seq. ID No: 328

>BH4syn_4MHHHHHHTSSTPVRTAYVTRIEHFSAAHRLNSVHLSPAENV
KLFGKCNHTSGHGHNYKVEVTIKGQINPQSGMVINITDLKKTLQVAVMDP
CDHRNLDIDVPYFESRPSTTENLAVFLWENIKSHLPPSDAYDLYEIKLHE
TDKNVVVYRGE

Seq. ID No: 329

>BH4syn_5MHHHHHHSSKEHHLVIINGVNRGFGHSVALDYIRHSGAHAV
SFVLVGRTQHSLEQVLTELHEAASHAGVVFKGVVVSEVDLAHLNSLDSNL
ARIQSAAADLRDEAAQSTRTITKSVLFNNAGSLGDLSKTVKEFTWQEARS
YLDFNVVSLVGLCSMFLKDTLEAFPKEQYPDHRTVVVSISSLLAVQAFPN
WGLYAAGKAARDRLLGVIALEEAANNVKTLNYAPGPLDNEMQADVRRTLG
DKEQLKIYDDMHKSGSLVKMEDSSRKLIHLLKADTFTSGGHIDFYDE

Seq. ID No: 330

>DAC_1MVDADIALNWAGGLHVCIVRPPGHHAEPGAACGFCFFNNVALAA
RYAQSLQSPSDPPLRVMILDWDIHHGNGTQHIFQDDASVLYVSLHRYDDG
TFFPSSEDAAHDKVGSGPGEGFNVNIPWNGGKMGDVEYLLAFHRIVMPIA
YEFNPQLVLVSAGFDAARGDPLGGCRVSPEGYAHMTHLLMGLAGGKVVVV
LEGGYNLTSISESMSMCTRTLLGDPLPFISDLHAPRPAALRAISSVLGVH
QKYWRSLCINVGPP

Seq. ID No: 331

>DAC_2MKTHPHPERPDRLQAIAASLATAGIFPGRCYPIPAREITKEELQ
MVHSLEHIETVELTGQILYSYFTPDGTNPHNRLKLDNRKLAGILSQRMFV
ILPCGGLGVDSDTIWNDLHSSNAARWAAGSVIDLAFKVVTRELKNGFALV
RPPGHHADPSTAMGFCFFNSVAIAAKQLQQKLNVRKILIVDWDVHHGNGT
QRVFYRDPNVLYISLHRHDDGNFFPGSGAADEVGANSGEGFNVNVAWAGG
LDPPMGDAEYLAAFRTVVMPIAHEFAPDVVLVSAGFDAAEG

Seq. ID No: 332

>DAC_3MMATEPIASGSGTMDIDSEKTPSTSQANPMADTFQTREAVLGLG
EVVEHVGGRWVAEQEWIRSPERKMAYTQGTKKKVCYYYDGDVGNYYYGQG
HPMKPHRIRMTHNLLLNYGLYRKMEIYRPHKANAEEMTKYHSDDYIKFLR
SIRPDNMSEYSKQMQRFNVGEDCPVFDGLFEFCQLSTGGSVASSVKLNKQ
QTDIAVNWAGGLHHAKKSEASGFCYVNDIVLAILELLKYHQRVLYIDIDI
HHGDGVEEAFYTTDRVMTVSFHKYGEYFPGTGDLRDIGAGKGKYYAVNYP
LRDGIDDES

Seq. ID No: 333

>DAC_4MDAGTRRVDDAAVPSTGPSASLLRSANMLSAAFGLTASLYSRLR
GVCSSRRALSTSARTSEAAGVGAKPGVAAALTVPSTGPSASEASPAALLR
IQVAEEWARASGLLDREDCQVGLAFDEAMHLHSGPAGHPERPARTKEILA
QLHASGLVRACAQVPSREATEEELLLVHDARHVERVLRHEAAGHKKAKAF
SFPFGPDTYVCEHTARCARLAVGCLLSLVDASLDPASPVRTGMAVVRPPG
HHATSDRASGFCLFNNVAVAARHLQRRHGLKRVAIVDWDVHHGNGTNDLF
TEDPNILFFSVHRFDNHGFFPGSGFLEDVGHAQARGYTVNVPLEKGYGDL
DIVHVVKYVLCPVLERFKPDAILVSAGFDAVKGDPLGECRVSPEAFGWMT
RCLHRLAQRYCDGRLFLVLEGGYNPDMIAQCCIECVQSLVAEAAGLRGPW
PEFPAVGVPLAEGAQLSAPSSAPTSAPGTPTSTSPASSPALSAAAPPLAS
PGSTPTSSPCLRPSGGEAPPRSPPSASASAGGGARQRARAPSSKTVRAVR
QLTEIHHLLPLELPVAPRPGDGPGAANKSARKNERRRLGRGRRGPEEEGA
SSDSSGWAIACGLSDAEPWPSPQASPVASLSQGASSLPTLELPPAFPSLD
GVGSTAGNSYLGTSGNVGIDAAGHSASSWLGSPTTAATAVAPPARGDRKV
KRR

Seq. ID No: 334

>DAC_5MVDADIALNWAGGLHGHHAGRGCSEGFCLLNNVAVAAAYARSAH
PEQVRRVLVLDWDVHHGQGTQEIFWRDPGVLYASVHRDGGEGFYPGTGAA
EQVGDGAGRGFTVNVPLPTGYGDGCLWAACAEVLLPAARRFRPDLILVSA
GFDAVAGDPLGGCRCTARGFGALTGELRKLAGELCSGRLLLALEGGYDLR
TLRACVGEVCQALAAPEPAEGGA

Seq. ID No: 335

>DAC_6MRNRSSGFCLVNNVAVAAEYARDRYPEVERVLIFDWDVHHGQGT
QQIFEQSPDVLVISVHRHDGHSFYPATGSAGEVGSGPGRGYSVNVALPAG
YGGAALWTACAHVLLPAARNFQPQLILVSAGFDAAASDPLGGCFVEPRVF
GALTAELRRLAAEVAEGRLILALEGGYNPEVLADCVDEVAAALVADASSS
GVEAFAEAPSWLAGSACFGAIRRTCEAHRMAPLRLPLPSSRIDRRRAAAR
QAEALSSPSSRDAGDTGGGEVSAHGATTTVTTSANLGAGTLAARPSSMVT
GEGRRANGQLVDVLGIALAGKPSASPWPEAQRTQGSAPGTPAPATGGALP
PAETAESPGSVASGAAVAQGPVECQAAARQAGECPGQAPAPAGAGAAPGG
RGVEAAAAQHGQDLAPAAGPGAAALVELQTGELVVRIAPLPRPKDVVVSA
EELWVWHDQGGPLGVQRWRFEGVRAENSGALRCAEFRSKRHELTVRLRLG

Seq. ID No: 336

>DAC_7MVRSSQATTFSSSPYFADRGIHGTAQPITERINPRKCTFHILPP
GLGGWLYLFPKMSKTVAYFYDPDVGNFHYGTGHPMKPHRLALTHSLVLHY
GLYKKMIVFKPYQASQHDMCRFHSEDYIDFLQRVSPNNMQGFTKSLNAFN
VGDDCPVFPGLFEFCSRYTGASLQGATQLNNKICDIAINWAGGLHHAKKF
EASGFCYVNDIVIGILELLKYHPRVLYIDIDIHHGDGVQEAFYLTDRVMT
VSFHKYGNYFFPGTGDMYEVGAESGRYYCLNVPLRDGIDDQSYRHLFQPV
IKQVVDFYQPTCIVLQCGADSLGCDRLGCFNLSIRGHGDCVQYVKSFNIP
LLVLGGGGYTVRNVARCWTYETSLLVDETISEELPYSEYFEYFAPDFTLH
PDVSTRIENQNTRQYLDQIRQTIFENLKMLNHAPSVQIHDVPSDLLSYDR
TDEPDPEERGAEDNYTRPEASNEFYDGDHDNDKESDVEI

Seq. ID No: 337

>DAC_8MWDVHHGQGIQYIFEDDPSVLYFSWHRYEHGSFWPNLSESDYDS
IGKGRGTGFNINLPWNQTGMGNADYVAAFFHVLLPLAFEFNPELVLVSAG
YDSGIGDPEGHMRATPECFSHLTHMLMHLAGGKLCMILEGGYHLRSLSES
VSMTVRTLLRDPVPRLSGEMTPCYSALESIQNTRHAHSPYWKCLLHDETR
LVEEISTKGLKAPGPLHVDASVVDEFLENHMKKILHPTPPITTMVVASVE
NTLNLPAGVQLEESTVTPEQARHAISVFNPDELNENVLNSVSKMLPALEK
LVN

Seq. ID No: 338

>DAC_9MKTHPHPERPDRLQAIAASLATAGIFPGRCYPIPAREITKEELQ
MVHSLEHIETVELTGQILYSYFTPDTYANQHSAHAARLAAGLCADLAKEV
FSGRAKNGFALVRPPGHHAGVRQAMGFCLHNNAAVAALAAQVAGAKKILI
VDWDVHHGNGTQEIFEQNKSVLYISLHRHEGGKFYPGTGAAHEFGTMGAE
GYCVNIPWSRGGVGDNDYIFAFQHVVLPIASDFAPDFTIISAGFDAARGD
PLGCCDVTPAGYAQMTHMLNILSGGKLLVILEGGYNLRSISSSATAVIKV
LLGESPGCNPKNFLPSKAGVQTVLEVLKIQMNFWPALGSIYSDLQTQWGM
YCMKTKKKQIKKRQRAAAPLWWKWGQKSFLYHLLNGHLHVKSKGC

Seq. ID No: 339

>DAC_10MFLVRHHLYKWKSHFRIDADGKFVEDQFFPKNLKSGRRFLRSI
GASITCSNGIGKDPYILSNEKISDARLIYAVAPAMGHNQESHPESHFRVP
AIVNALEKMEMTPKFRGSEIIELQSFKPALVDDIASVHARAYVSGLEKAM
DQASQQGIIFIDGSGPTYATATTFHESLVAAGAGIALVDSVVAASKNHLD
PPVGFALIRPPGHHAIPLGPMGFCVFGNVAIAARYAQRAHGLKRVFIIDF
DVHHGNGTNDAFYDDPDIFFLSTHQDGSYPGTGKVDEVGRGDGEGTTLNL
PLPGGSGDIAMTTVFDEVIAPCAQRFKPDIILVSAGYDGHVLDPLASLQF
TTGTYYMLASKIKLLAKDLCGGRCVFFLEGGYNLESLSYSVADSFRAFLG
EQSLASEFDNPAILYEEPSTKVKQAIQRVKHIHSL

Seq. ID No: 340

>DAC_11MVDADIALNWAGGLHHAKKSEASGFCYVNDIVLGILELLKVHR
RVLYVDIDVHHGDGVEEAFYATDRVMTVSFHKFGDFFPGTGHIKDTGWGP
GKNYALNVPLNDGMDDESFRGLFRPIIQGVMEVYQPDAVVLQCGADSLSG
DRLGCFNLSVKGHADCLRFLRSFNVPLMVLGGGGYTIRNVARCWCYETAV
AVGVEPDNKLPYNEYYEYFGPDYTLHIEPCNMENLNTPKDMEKIRNMLLE
QLSRIPHVPSVPFQTTPPTTQVPEEAEEDMDRRPKCRIWNGEDYDSDPDE
DEKPRHTEPNSELRDVVDEMDEDKREEHPPS

Seq. ID No: 341

>DAC_12MCSDANGKVGNISVMSTEGISQVESKKARLNGLLTLEDIYNLP
DELDDDEDDSDWEPLLEPLAVRKWFCTNCTMVNFDGFDFCETCEEHKESG
ILKQGFFASPALQGTRSTQIESEVIERYTESICDISASALSTVVGFDERM
LLHSEVVLKPHPHPHPERPDRLRAIAASLSTAGIFPGKCHPIAAREITQE
ELLKVHSLEHVEAVEVTRQMLSSYFTPDTYANEHSAQAARLAAGLCADLA
SEIYSGRAKNGFALIRPPGHHAGVHQSMGFCLHNNAAVAALAAQVAGAKK
VLIVDWDVHHGNGTQEIFERNKSVLYVSLHRHEAGKFYPGTGAAHEVGTM
GAEGYCVNVPWSRGRVGDNDYIFAFQNVVIPIAHEFSPDFIIISAGFDAA
RGDPLGGCDVTPAGYACMTHMLSALAGGKMLVILEGGYNLRSISSSATAV
IKVLLGEKPKCQFENIEPSASGLQALLEVLKVQTNFWPCLSSKLTQLQSC
WEAYLSGRKKQKKRRFRTVAPPPIWWAWGRKRFLYFLRCQRFRMKP

Seq. ID No: 342

>DAC_13MAGAEELHVFWEEGMLKHETGRGVFDTGSDPGFLDVLEKHPEN
ADRVRNMVSILKRGPIAPFVSWHQGRPASLPELLSFHSSEYIEELEEADR
AGGKMMCCGTFLNPGSWNAALLAAGTTLSAVKYILDGHGKIAYALVRPPG
HHAQPTQADGYCFLNNAGLAVQLALDEGCRKVAVIDIDVHYGNGTAEGFY
CSNKVLTISLHMNHGSWGPSHRQSGTHDELGDGDGFGYNMNIPLPNGSGD
RAYEYAMQELVVPAVQKFGPDMIVLVVGQDSSAFDPNGRQCLTMDGYRQV
ARIVRGLADMHCKGKLLVVQEGGYHITYAAYCLHATLEGALNLPSPLLSD
PIAYYPEDEGFAVKVIDAMKEHYKSNVPFLKEIN

Seq. ID No: 343

>DAC_14MGFCIFGNIAIAARYAQRVHGLKRVFIIDFDVHHGNGTQDVFY
EDPDIFFLSTHKEGSYPGTGKIHEVGCGPGEGTTLNLPLPGGTGDVAMRT
VFDEVIVPCAQRFKPDIILVSAGYDAHFLDPLANFQFKTATYYTLAANIK
QLAKELCGGRCVFFLEGGYNLKSLSYSVADSFRAFLGEPSCASDVDPTFL
YDEPSTKIEQAIDKVKAIHSL

Seq. ID No: 344

>DAC_15MEQLWVPSLPILGGRILPMLRHYCGFGSHHPLTWRSLQITGRK
QKHNGCWIAYCLPSHNGTSISDTNGVRKDLALPDNLLRDAHILYCTSPAM
GHNKEAHPETNKRVPAIVDALEKLELTSKHRGSQVLEIQDFQPASLDDIA
LVHSRSYITGLEKAMSRASDEGLIFIEGTGPTYATQTTFQECLLSAGAGI
TLVDSVVAASKLGPKPPLGFALVRPPGHHAVPEGPMGFCVFGNIAVAARY
AQNQHGLKRVMIIDFDVHHGNGTCDAFYEDPDIFFLSTHQLGSYPGTGKI
HQVGQGNGEGTTLNLPLPGGSGDYAMRCAFDEVIAPAAQRFKPDIILVSA
GYDAHALDPLAGLQFTTGTFYMLAARIREVAAELCGGRCVFFLEGGYNLE
SLSSSVADTFRAFLGEPSLAARFDDPAMLYEEPTRKIREAIDKAKHLHSL

Seq. ID No: 345

>DAC_16MMATEPIASGSGTMDIDSEKTPSTSQANPMADTFQTRRPRASS
LPLQPSNLKVGYIYSSEMMNHFCPGGHPEQPLRIQQIWATIVNEQLHKRM
KWMPIREVKKGEALLVHSEDHWNKVIAIQYLTDQQRADSVDYYEQMSLYV
MSGTTRSALLSCGGVVEACLAVARNELKKTFAIVRPPGHHAEPDEHMGFC
FFNNVAVAARVVQQRTKLKKILILDWDVHHGNGTQRAFNDDPSVLYISLH
RYEQGTFYPCGPFGSLTSCGEGPGTGFSVNVPWPCAGMGDAEYIYAFQKV
ILPIATEFAPELVIISAGFDAAAGDELGECLVSPAGYAHMTHMLAGLAGG
RMVVALEGGYNLDSISQSALAVTKVLLGEPPDELPPLKANEEGTETVWLV
AREQSKYWKSVDPKACEPQADVEPISFSVPEILKAHRQHYLYTKHDMMQV
PMMTPELEEKFSSQIMCTSDIFESKTLVIFVHEFGNLRLELESSTTCDVH
LERSYLIDFSKELVGWVKSEGYSLLDANLYPKPSTTPTPNLRHKTMEEVG
RDVLVYLWDNYVQLSGAERVILIGHGPGCKPLVDLLNRRTTSVTKSAKAI
IQVVGSQRMPSYPSDVDDARPWYQKSSLVIVPQSHPVMGPHIKPKDIRRH
GVMVPIDETRQIKLITRALPAIKQFVQETLSSFPLANRTNRP

Seq. ID No: 346

>DAC_17MSKRKVAYFYDPDVGAYTYGWSHLMKPHRMRITHELATAYGML
DKMHVLRPKRATPEAMTAFHTDEYVQFLHSVTPETADKLTGQKTRFLVGD
DNPAFEGVFEFCSISAGGSIGAAERIASGAADIAINWAGGLHHAKKREAA
GFCYINDIVLGILELLRTYPRVLYIDIDCHHGDGVEEAFYTTDRVMTCSF
HKFGEFFPGTGTQEDTGTGKGKGYSVNVPLKDGIQDESFKSVFDPVISKI
LEVFQPSAVVLQCGADSLAGDKLGCLNLTMQGHAHCVQFLRKSNIPLILL
GGGGYTVKNVARAWTYETACAIGIENEIDLNMPWSQYFEWFGPTYRLEVP
ENNMEDMNVKEGTLDHVRTTALAQLQQLASRCAPSVQMQDVPRTSLGGHL
GFKRDKREHRDELDERLAQHTRYLYDLQESESESEDTESSDSDASSVSFV
NNWRRAPHRANSLPRILSGRHSSNPPGHISASERRRMSIVTGKYFDIPIH
ESGYNHYEYGAAPTKSSKRIFFQSGLDIYNDDNDFEGIINARTSVSNGFG
NGIHDLHGLMERGGRSLNENLEDGDDEVEGEEYEDDAAMSDS

Seq. ID No: 347

>DAC_18MEEHFWDVLYKDKYSKLLSKARDFLDDTGGPGDDVLVFISCGM
DACEHEYESMSRHNRKVPASFYHRFARDACAFSDRYAGGRLISVLEGGYS
DRALISGAMAHLSGLVDTPDGIQVDEQWWNIPNLVKLEAATKKRRGGRPS
LPAKGSVEPWIERTLSIFSSIDGSASTTSSRSTFIPPSSRTLRDRTKGRE
AMPKSPPASSASTKPVSRSKVKPGANIKSGDESFASTGSSPLTSPSPSSS
EDEAPPIKRL

Seq. ID No: 348

>DMAT_1MTIINSRIIDIRQSTFEESIPDQVTAGLSTTPKTLPALLFYSG
EGIRHWIEHSTAADFYPRHEELRILRARAAEMVDSIANNSVVVDLGSASL
DKVLPLLEALEASKKNITFYALDLSFSELQSTLQSLPYEQFKFVKIGALH
GTFEDGVQWLKDTPGVQDRPHCLLLFGLTVGNYSRPNAAKFLQNIASNAL
AASPVQSSILLSLDSCKMPTKVLRAYTAEGVVPFALASLDYGNTLFAPNK
MGEKVFQPSDWYFLSEWNYMLGRHEASLITKGKEVRLGGPLNDIVIEKHE
KIRFGCSYKYDTDERQVLFGSAGLTDVKEWSVEGCDVSFYQLQMCPN

Seq. ID No: 349

>DMAT_2MTISAPPIIDIRQAGLESSIPDQVVEGLTKEVKTLPALLFYST
KGIQHWNRHSHAADFYPRHEELCILKAEASKMAASIAQDSLVIDMGSASM
DKVILLLEALEEQKKSITYCALDLSYSELASNFQAIPVDRFHYVRFAALH
GTFDDGLHWLQNAPDIRNRPRCILLFGLTIGNFSRDNAASFLRNIAQSAL
STSPTQSSIIVSLDSCKLPTKILRAYTADGVVPFALASLSYANSLFHPKG
DRKIFNEEDWYFHSEWNHALGRHEASLITQSKDIQLGAPLETVIVRRDEK
IRFGCSYKYDKAERDQLFHSAGLEDAAVWTAPDCDVAFYQLRLRLN

Seq. ID No: 350

>DMAT_3MSKPNVLDIRLATFEDSIVDLVINGLRKQPKTLPALLFYANEG
LKHWNHHSHQPEFYPRHQEVQILKKKAQEMAASIPMNSVVVDLGSASLDK
VIHLLEALEVQKKNISYYALDVSASQLESTLAAIPTQNFRHVRFAGLHGT
FDDGLHWLKEAPEARDVPHTVLLFGLTIGNFSRPNAAAFLSNIGQHAFQG
KSGDQCSILMSLDSCKVPTQVLRAYTCEGVVPFALQSLTYANGLFSEKNK
TQASGDVQHKVFNLDEWYYLSEWNFVLGRHEASLIPRSKDIKLLPPLDGI
LVSKDEKVRFGCSYKYDQEERMELFAAAGVKNEVTWSDEGCDVAFYQLKL
S

Seq. ID No: 351

>DMAT_4MGSINPPQILDIRRSKFEESIPKQVEAGLLSSPKTLPALLFYS
TEGIQHWNRYSHASDFYPRHEEIQILKDKATDMAASIADGSVVVDLGSAS
LDKVIHLLEALEAAQKKVTYYALDLSFSELTSTLQAIPTDQFVHVQFSAL
HGTFDDGLQWLKETLVIRDQPHCLLLFGLTIGNFSRPNAAKFLHNIASHA
LVESPSQSSILLTLDSCKVPTKVIRAYTAEGVVPFALESLKYGNTLFQQD
AGENVFDPEDWYFLSEWNYVLGRHEASLVPRSKDIKLGRPLDKIVVGKHE
KVRFGCSYKYDSEERKELFGTAGLRDVKSWSKEGCDVAFYQLKCCPN

Seq. ID No: 352

>DMAT_5MPALPVIDIRSNHVEDSLPEQIIKGLTSQPKTLPPLLFYSNEG
LEHWNHHSRQPDFYPRRQEIEILKQGGNDIARSIAPSSVILDLGSANLEK
VGYLLEALEAQEKDVLYFALDISAPQLATTLKEIPSSNFRHVRFAGLHGT
FEDGLRWINETPEIRDLPHCVLLLGLTIGNFSRQNAAAFLQNIANHALTG
ASKNKSSILLSLDSCKVPTKVTRAYTSDGVVPFALQALTYAKALLCDRID
NGIDEKVLSCNLRPEHWHYLSEWNFALGRHEASLIPRFGDVCLGSMLQDI
IVKKEEKVRFACSYKYDAKERQKLFLDSGVDQGMVWTNEGCDVAIYELKL
A

Seq. ID No: 353

>DMAT_6MLYKPKVLDIRSGSVEDSLRHSVMDGIREDPRTLPTLILYGPE
GLQHWDDHSHAPDYYLRHEELHILRSRAYEMAETIADNTAMVDLGSAQVS
RFHESPCLLAPTLSLDKAALLLDALEVQAKNVTYYALDLDHAELQKTLCR
LPLGKYKHVQCVGLQGTFEDGLEWIKNDPEQSRRPHCLLFLGSTIGNFSR
ENAARFIRSMASSAFLSESAKSSIILSIDSCKLPTKVLRAYNSEGVVPFA
MAGLKHASAILCEAACRQEDAVTETFLPDDWYYLSHYNHVLGRHEASFTP
RNRDIQLGSPLEDVVIRLGETIRFGYSHKYDFAEIEQLFREAGVAAVNSW
GAVGCDLSFYQLGTA

Seq. ID No: 354

>DMAT_7MAAPSVIDIRSHLVEDSLPDQVVKGLGSDPKTLPALLFYSNEG
LEYWNHHARQPDFYPRHQEIEILKRKGDEIARSVAPNSVILDLGSANLEK
VTYLLEALEAQAKNVTYFALDLSAPQLMSTLKAIPTTKFRHVRFAGLHGT
FVDGLRWISETPDIRDLPHCVLLFGLTIGNFSRPNAATFLRNIASQALRG
ASEDKSSIFLSLDSCKVPTQILRAYTSDGVVPFALQSLAYAKTLFCEQTQ
NDFNEKPSSCHLNPDDWHYHSEWNFVLGRHEASLIPRLNDIHLGPLLHDI
VVKKDEKVRFGCSYKYDDLERDKLFVDAGVKDEMAWTNEGCDIAIYELKS
M

Seq. ID No: 355

>FEX1MIFNPVISNHKLSHYIHVFCTFTTFCILGTETRQAITALSTYTPA
FVTAPTVLWSNCSSCMLMGIMQSLNAYTWMKDHQVLFLGVTTGYCGALSS
FSSMLLEMFEHSTNLTNGNIANHTKLPNRAYGIMEFLSVLLVHLMVSMGS
LIFGRQLGKEVIVAYGSSSFSKPYTPPSDTVKENAGDVDTQEMEKNILEF
KFKTPAPFFKKFFDIVDKLAYALAFPLIILFVVLCAYYENYSRGKWTLPC
LFGIFAGFLRYWLAEMFNKTNKKFPLGTFLANVFATLLIGIFTMVQRGKK
HFSTDVPIVNSLNSCHIVSALISGFCGTLSTISTFINEGYKLSFINMLIY
YTVSIAISYCLLVITLGSYAWTRGLTNPIC

Seq. ID No: 356

>IDI1_for_fusionGGSGGSSGSGGSSSTADNNSMPHGAVSSYAKLVQ
NQTPEDILEEFPEIIPLQQRPNTRSSETSNDESGETCFSGHDEEQIKLMN
ENCIVLDWDDNAIGAGTKKVCHLMENIEKGLLHRAFSVFIFNEQGELLLQ
QRATEKITFPDLWTNTCCSHPLCIDDELGLKGKLDDKIKGAITAAVRKLD
HELGIPEDETKTRGKFHFLNRIHFMAPSNEPWGEHEIDYILFYKINAKEN
LTVNPNVNEVRDFKWVSPNDLKTMFADPSYKFTPCFKIICENYLFNWWEQ
LDDLSEVENDRQIHRML

Seq. ID No: 357

>INMT_1MAAPHTSQQDYIDNFNARDYLQTSYTPGKGILFGEWIEFATQN
LHETFTTGGVRGDTLLDFGTGPTIYQLISACEVFDKIIVSDFLEQNRAEF
RKWLNKDPDAFDWTPIIKGVCELEGNREDWEKKATKLRSKVKEVLKCDAL
KRNPYDPIVVPPVDCLLSCLCLEAPCKDIKSYCEVLKNFQSLIKPGGHLL
ILSGLNATFYYVGKTYFSSMTTKKEELEMAFKEAGYIIKKAVYTPRADKS
KIDVADYEGHYFIHAHKPK

Seq. ID No: 358

>INMT_2MAAPHTSQQDYIDNFNARDYLQTSYTPGKGILFGEWIEFATQN
LHETFTTGGVRGDTLLDFGTGPTIYQLISACEVFDKIIVSDLLEQNRTEF
QKWLNKDPDAFDWTPIIKGVCELEGNRENSEKKAEKLRSKVKQVLKCDAL
KRNPYDPVIVPPADCLLVCLCLEIPCKDMKSYCNVLKNFKDLLKPEGQIL
ILGTLNGTYYHAGKKRFSLLSSKKEDLEMAFKEAGYIIEKAVYTLRADKS
NIDVADYEGHYFIHAHKPK

Seq. ID No: 359

>INMT_3MSDFTNTREYEEQFDPRLYLETYFHLGSGSLADDFLRFVLDNF
NKTFKSGAVKGSTLIDIGTAPSIYQLLSACESFDDIIVTWHTNRELKELQ
KWLNSEADAFDWSSIVKHVCEIEGNRMAQKEKEEKLKGKIKQVLMCDVSK
SNPLSPHEVPKADCLLTTVCLEAACKNYESYGTALKNLSNLLKPKGHLLM
AGDLGANYYEVGSNKVFSLPVNEKFLKKVISESGYEIIQLVSFGKPENAD
FETSDYEGFYFVHAQKV

Seq. ID No: 360

>INMT_4MDCLISCLCLEAPCKDLEDFTNTLKKFKELLKPGGHIIIQSVL
NCSLYFVGNKSFSCLSITKDELEQAFKEAGYEIVKLKVVPRSEKIWANVS
DHSEYYYIHARKPQ

Seq. ID No: 361

>INMT_5MSDFTGKNEYQTFFNPKAYLESYYQLGSGSMGDEYLQFVLKEL
AETFNPGKVKGDTLIDIGTGPTIYQLLSACEAFKNIIVSDFTDKNREEFN
VWLKNQPGAFDWSPVVKHVCRLEGDRIPWEQKEERLRKTIKQVLKCDVFN
INPIDPVTIPQVDCLLSCLCLEGACKDFESYITALKNMTTLLKIGGYLVM
TGDLGNTYYMVGDVKFSGLNLNENFLREAITGAGYVIESFQQSKKTEDSV
EDKADFTAYYVIVARKERNV

Seq. ID No: 362

>INMT_6MESGFTSKDTYLSHFNPRDYLEKYYKFGSRHSAESQILKHLLK
NLFKIFCLDGVKGDLLIDIGSGPTIYQLLSACESFKEIVVTDYSDQNLQE
LEKWLKKEPEAFDWSPVVTYVCDLEGNRVKGPEKEEKLRQAVKQVLKCDV
TQSQPLGAVPLPPADCVLSTLCLDAACPDLPTYCRALRNLGSLLKPGGFL
VIMDALKSSYYMIGEQKFSSLPLGREAVEAAVKEAGYTIEWFEVISQSYS
STMANNEGLFSLVARKLSRPL

Seq. ID No: 363

>INMT_7MKGGFTGGDEYQKHFLPRDYLATYYSFDGSPSPEAEMLKFNLE
CLHKTFGPGGLQGDTLIDIGSGPTIYQVLAACDSFQDITLSDFTDRNREE
LEKWLKKEPGAYDWTPAVKFACELEGNSGRWEEKEEKLRAAVKRVLKCDV
HLGNPLAPAVLPLADCVLTLLAMECACCSLDAYRAALCNLASLLKPGGHL
VTTVTLRLPSYMVGKREFSCVALEKGEVEQAVLDAGFDIEQLLHSPQSYS
VTNAANNGVCCIVARKKPGP

Seq. ID No: 364

>INMT_8MSDIDDGALASAQAIVDGNRLAGQIELRQQPDPDRVFAGVLRQ
GEAVAFCVCNPPFHESLEHARRAAGAKWQRLGRAVQGKEMNYQGSPAELC
CNGGEVGFVTRMAEESAQPRRQRACVWFSAMLSRESSIAPVRERLGELGA
RRRAWELRQGRTTKWVVAWTFYPRGERDQRLREMAQRRADPEARAEAGAE
AATARDVGAGGDGADGVGGSLVRRSAGAGGSAA

Seq. ID No: 365

>INMT_9MDFTGGEIYQSSFDPKAYLASFCSLGSGRDDILMFRLKKCFET
FGPGGLRGDVLVDIGTGPAIYHLLSACESFPYIIATDFTDNNRQELEKWL
RREPGTFDWLETVKIVCDLEGDSRDDWVEKEDKLRSRIQKVLKCDVTKTN
PLDPTVIPPADCLITALCLETACTDIDTYFCSLRNITTLLKPGGHLVLIG
VLGNSFYKVGEKKFYCLSLDEQTVRNAVIDAGYSIKDLELYYLPNPASCA
HITDTYANIFLVAQKNET

Seq. ID No: 366

>INMT_10MEIVSTSYNHIYDNFDARKYLDRYYGLASETQEIEEESVFLL
TFLSNVFSSGRVKGHSFIEIGVGPSIHSILSACEVFEKIYLTDYSQGNLN
EIEKWLNSENDAFDWTPYIRFVCDLENNGSTPKGKKEKLRRAVSLMKCDV
NLSNPLHPHSLPLTDCLLTASCLSATCKTFTDFKMSLKIIVSLIKPGGHL
ILIDYLRASYYWVGEVKLPILSLDEHVVREAVVESGCKIEEFKWFKEFHM
PDELSDCKTVFSLLAQKL

Seq. ID No: 367

>INMT_11MDSSNYKLYHVHEFNSRSFLDNYFSDGPQMTFVDDTLVFPIE
NLKKTFAEGHIKGDVMIDLSIGAMVHHLYAACEFFKDIIVLKASDRCIME
LKRWVGTRTGAFYWGHATKLHADTEGNSELLQDKEEKVRSAIQHVVKCDV
TKELMTDPIVLPPADCIISAWLLDAISSNQDDFITYLRRFIKLLKPGGHL
ILIGALEQTSYSVGNEKYQFLTYNEDFARKALIAEGLVIDDCKIKKRTAK
SDLADYKSILYLVSHKK

Seq. ID No: 368

>INMT_12MDPCLNLYYPSHEVNAKRLLHEYFSQNVPYSIFKESTINIMK
CCYKAFSSGLLSGTTLIDISVGPSIVHLLSVCEFVEEISILKVNDASIRE
LELWKNKDPETFDWTHTLKLFMELKGTSRDGWKDAQEMLRRKVKHIVKCD
FSKSNLTKPFALPRADCVTCIWGLETISRDHDEWKTTLRKISDLVKLGGH
VLIHADINASYFKIGEDKYHLFNFDDAFLRKTLTDGGFAIVHYENLEREA
CTDCLDHSHK

Seq. ID No: 369

>INMT_13MELKRWVDTRTGAFDWSHAAKLHVDTEGNSDELQEKNEKVKS
AIQHVVKCDLEKENMTHPIVLPPADCIISFALLDVISKDKDDYIKYLRKF
SKLLKPGGHLILIGDLDTTYITVGKHKVHYLTYDEEFVRNALAGEGFVID
CCKVKERTVESDLCDYKGMIFIVAHKEK

Seq. ID No: 370

>INMT_14MELKRWVDTRTGAFDWSHAAKLHVDTEGNSDELQEKNEKVKS
AIQHVVKCDLEKENMTHPIVLPPADCIISFGFLDVVCKDQEDYIRYLRKF
SRLLKPGGHLILIGGVDATYFTVGKEKHHFFTYDEAFVRKALEGEGFVID
DCKVKKRTAVSDFTDYKGSIFIAAHKEH

Seq. ID No: 371

>INMT_15MSDFTNTSEYEEQFDPRLYLETYFHLGSGSLADDFLRFVLDN
FNKTFKSGAVKGSTLIDIGTAPSIYQLLSACESFDDITVTWHTNRELKEL
QKWLNNEADAFDWSSIVKHVCEIEGNRMGQKEKEEKLKGKIKQVLMCDVS
KSNPLSPHEVPKADCLLTTVCLEAACKNYESYGTALKNLSNLLKPKGHLL
MAGDLGANYYEVGSNKVFSLPVNEKFLKKVISESGYEIIQLVSFGKPENA
DFDTSDYEGFYFVHAQKV

Seq. ID No: 372

>INMT_16MALQERQEPDVYQENFEPTSYLEYYRMNQDPVGDEVLHFLLK
HYNATFKPGGLEGKLLIDIGSGPTIYQFLSACESFQEIIATDYTDKNLQE
LEKWLKKMPGAFDWSPVVKYVCELEGNRDKWAEKEERVRRAVTQVLKCDV
LKERPLEPAVLPPADGLISSLCLEAACPTPQACRDALRHLRTLLRPGGHL
VLSGGFETTFFMVGDKRFSTLPLNEKFLREALQEAGFIIEKLEKVTRAAE
THLDNRSDYTGLFFLVARRGD

Seq. ID No: 373

>INMT_17MDKISAPFFSGTSPAAASVAGVDEDDRLCFQAQELMFAYNIS
MVLRAAIQLGLLDALSAAGGKALTPNELVENVETSSNKAEAAAAVDRILR
YLSCFNVVTCSSEAAGPDGTLVRRYTTGPLCRWLTKDRGDGTLSPFAVFV
VDPDHLFPWHHIAEAVTAGGPSAFERTQKWPYYEYMGKNQRLGTLFDNAM
AQHSVILVTKMLERFKGFDGVQRLVDVGGGTGSTLGMITSKYKHMTGINY
DLPHVIAQGLPLPGVEHVAGDMYESIPTGDAVLLQWITLMLNDDEFVKIL
SNCHNALPKDGKVIVVDGILPENPDSSLTARDAFTLDIIMFVLFKGAKQR
TEKEFARLAKQAGFTGGIKKTYIFFNFYALEFTK

Seq. ID No: 374

>INMT_18MDANKRYHGPPVLLGVVRDSEKFDFCMCNPPFFETMEEAGLN
PKTSCGGTPEEMICPGGEKAFITRIIEDSAVLNQSFRWYTSMVGRKSNLK
SLISKLREVGVTIVKTTEFVQGQTCRWGLAWSFVPPVRKIVSPHVAEKNI
ISFMLEVWVPGFSICRVGDDLVPKSKSPHLSPILGTKN

Seq. ID No: 375

>INMT_19MEEAGLNPKTSCGGTPEEMICPGGEKAFITRIIEDSAVLNQS
FRWYTSMVGRKSNLKSLISKLREVGVTIVKTTEFVQGQTCRWGLAWSFVP
PVRKIVSPHVAEKNIISFMLEGLQRQFSAIHVLQSIESFFRTCGASSELN
ASSFTVDITATNDHCKAILNNELQSIDEATSCEHVPETSNSSSSLHPHSN
GLGFRISVYQQIPGTLLVKGSLQHKNNPVSGAFSLIIQRLEEDLKYKFCR

Seq. ID No: 376

>INMT_20MNRSNYIHWIEDLLASDITEKNEANGGKVRGFDIGTGANCIY
PLLGASLLGWSFVGSDVTEVALDWAEQNVRSNPHISELIEIRRVDVDDPA
SSSGTVESSGGSRMEDSSQGQCDVVELASLEMKEFCDVGVTCKGGTDKNQ
RRYDEAKHSNVAKGYQGPPILLGVVKEGEKFDFCMC

Seq. ID No: 377

>INMT_21MEEAGLNPKTCCGGTPEEMVCQGGERAFISRIIEDSATLKQS
FRWYTSMVGRKSNLKFLMSKLREVGVTIVKTTEFVQGQTCRWGLAWSFMP
TAKRSVPSHVAEKRNLSFMLEGLHRQTSAFNVLQSMESFFSHFGALCKSN
PSSFTVDVSVSSDHCDAILKSDVEKLDEASSHSCVAESPGSASSYDPMVV
SFRLSVFQQIPGTLLVRGSLQQRDSPLSGAFLSVFQQLEKFLKHKFCRER
GLQFNQR

Seq. ID No: 378

>INMT_22MATEIDDESYESARRNISNNNMQSRIHVEKASPDQSILFPLE
DDRTFEFTMCNPPFYGSAAEVVQSAEAKEFPPNAVCTGADIEMIYPHGGE
EGFVMKILDESERFMTRCKWYTSMLGKMSSVATIVEVLRQRSITNYAVTE
FVQGQTRRWAIAWSFADTRLPDTMARIQSISPKHALYPCMPPKNTLVQAF
PGPATHLVSTKLIETLHGIEGVSYTTTSLNSFFVEARQNTWSRSARRSRA
NKNSSKKPDPSSLDADDILSGSQPALTCSCRVLADTAHADPVNVVENQWI
FGNDRALFESFVGHVSRKVGMGLRDVK

Seq. ID No: 379

>INMT_23MLLESYKTFEPANYLQEYYSTVDLENRSLLAFFAEAYKGIDP
NSVMLEFSGGPSLYSLITAAAHVKEIHFSDFLERNVEEIKLWKRFRHRSY
IWINFFKEALMAEGLSEVSTDDILEREELLSKKLSDFLLCDAFNRHPLGQ
RCYQRYDVVAANFVAESITPSLKTWEEVVNNICSTLKPSGTLIMTAIQGA
SFYCVENHRYPAIAVTPEDVIRVLSYQGFDVDNLLMRHIPAEITDISAKD
YKGYQGMLFVKATR

Seq. ID No: 380

>INMT_24MESGFTSKDTYLSHFNPRDYLEKYYSFGSRHCAENEILRHLL
KNLFKIFCLDGVKGELLIDIGSGPTIYQLLSACESFTEIIVTDYTDQNLW
ELQKWLKKEPGAFDWSPVVTYVCDLEGNRTKGPEKEEKLRRAIKQVLKCD
VSQSQPLGGVSLPPADCLLSTLCLDAACPDLPAYRTALRNLGSLLKPGGF
LVMVDALKSSYYMIGEQKFSSLSLDREAVRDAVEEAGYTIEQFEVISQSY
SSTTSNNEGLFSLVGRKPVGSE

Seq. ID No: 381

>INMT_25MEIVSTSYNHVYENFDARKYLDRYYGIAPEAEKIDEESVFLL
TFLSNVFSSGRVKGHSFIEIGVGPSIHSILSACEAFEKLYLTDYFQGNLD
EIKKWLNSENDAFDWTPYIRFVCDLENNGSTPREKKEKIRRCVSLMKCDV
NLSNPLHPHSLPLTDCLLTACCLTSTCKTFTDFKMSLKTIVSLIKPGGHL
ILIDYLRASYYWVGEAKLPLLSLDEHGVREAVEESGCKIEEFQWFKEFHM
PDEVSDCKTVFILLAQKL

Seq. ID No: 382

>INMT_26MRNLHETFGPGGVKGDILIDFGAGPTIYQLLSACEVFNTIIT
SDFLEQNREQLKKWLRKDPDALDWSNFAKYVCELEGKSDNWEKKEETLRR
KVTKVLKCDALAEKPYDPVPMPEADCLISCLCLEVACKDLEDF

Seq. ID No: 383

>INMT_27METPFTSQQTYVDEFKASDYFKTYYVAEGGIANEEWTDFALR
TLHETFTKGGVKGETLIDFGAGPTIYHLLSACEVFDKIITSDYLEQNRAE
LEKWLKKDPSAFDWTPIIKFVCELEGNRNYEKKAEKLRNKVKEVLKCDAL
KRNPFDPIVLQPADCLLTCLCLEAPCEDMKSYFNVLKNFKDLIKPGGHLV
ILSVLDATFYYVGDKYFSSMTTRKEELEQALKEAGFEIEKAVYTTRKDRS
QMDIADYQGFYYIHARNPK

Seq. ID No: 384

>INMT_28MEGSFTGGEEYQKYFQPRDYLTTYYNFDGSPTPEAEMLKFNL
ECLHKTFGPGGLRGDTLIDIGSGPTIYQVLAACESFRDITLSDFTDRNRE
ELEKWLKKEPEAYDWSSVVKFACELEGDSGRWQEKEKKLRSVVKRVLKCD
ANLASPLAPAALPPADCVLTLLAMECACCSLDAYRAALCNLASLLKPGGH
LVTTVTLGISSYMVGKREFSCVVLEKEGVEQAVLDAGFDIQQFLHIPKCY
SATIAANNGVCFIVARKKPAP

Seq. ID No: 385

>INMT_29MEGSFTGPDEYQKYFSPKDYLDTYYSFEHGPSPETEMIKFSL
QFLHKVFGPGGIRGETLIDVGSGPTIYQVLAACEAFSDITLSDFTDRNRE
ELQKWLRKDAGAFDWTPVLKFACELEGNSSHWQEKAEKLRATVKRVLKCD
VNLGKPLAPVELPAADCVLTLLAMECACCSLAAYRAALCNLGSLLKPGGH
LVTSITLQISSYMVGKHQFSCLYITKEEVERAILDAGFDIEQLLHSEQSY
SATIAPNKGICFIVARKRSGP

Seq. ID No: 386

>INMT_30MDAQLTQLRNADVSWAAFDPIAYVDHNYRDLQAEDAEILHLV
RDHFGDHFRKQGGGPVSGIDVGAGANLYPALAMMPWCEEITLFERSPANV
RYLKSQVDSYDANWDQFWDALCAHEAYNSLGTDPRERFGKVVWVEQGDLF
DLARYERRWSMGTMFFVAESMTTSYQEFMLGVERFMRALSPGAPFAAAFM
EHSKGYHAGEHFFPACDVGESEVRASLEGFAGDFKVQRLESAAQLRDGYS
GMIVAY

Seq. ID No: 387

>INMT_31MSDFTNASEYEKQFDPRLYLETYFHLGSGSLADDFLRFTLGN
FHKTFTEGEVKGTTLIDIGTAPSIYQLLSACEYFQDITVTWYTNRELQEL
QKWLNKDPGAFDWSSTVKHVWELEGKRGMLEEKEEKLRGMIRQVLLCDVS
KKNPLEPVTLPKADCLISTVCLEAACRNYDSYRTALKNLSTLLKPGGHLL
LAGDLGANYYEVGSNKVFSLPVNETFLRKAVNESGYVINKLVSFGKPEDA
GYDTSDYEGFYFIHAQKC

Seq. ID No: 388

>IOMT_1MSSKLDNQNITANEEEEAFHQAMQLAMSTILPMVLKAAIDLDL
LEIIAKAGPAGCKLSPIEIASHLPTKNPDASSIIDRILRVLASHSILTCD
LATNEDGHVQRLYGLAPIAKYFLHNDDGISLIPTLTISTDKYLLGAWYHL
REATLEGGAIPLVKAYGMDLFELAAKNDEISGKFNNTMGNQTAIIMKKVL
EIYKGFEGINQLVDVGGGLGINLKLIVSKYPQIKGINFDLPHVVKDAPHF
LGVDHVGGDMFIEVPQGEVIFMKWILHDWGDDRCLKLLKNCYNALPKFGK
VVVVELVVPESPMTDIVTKNTLTLDAGLFIVVPGAKERTKEEYEALAKKA
GFSTFRLVCRAYSYWVMEFHKNVIV

Seq. ID No: 389

>IOMT_2MGSQAEVGKAMTEEEACEFAMQLVSSSILPMTLKAALELELLE
IMATAGEGAQLTPAEIAAQLPTSNPDAPIMLDRMLRLLACHSVLTASTYT
DDDGKVRRRYGLAPVCKFLVRNQDGVSTAALSLVNQDKVTMESWYYLKDA
VLEGGIPFNRAHGMTAFDYPGTDPRFNRVFNQGMSNHSTLTMKKILETYT
GFRGLHSLVDVGGGIGAILSLIVAKFPHIKGINFDLPHVIDDAPQFPGVE
HVGGDMFASVPTAEAILLKLILHDWGDEHCVKLLKNCCKALPEDGKVVVV
EAILPEGIDHSYASACVYQVDMIMLVTNPGGKERTLKEFEELAKAGGFAG
IRPICCVYGSWVMEFYKKM

Seq. ID No: 390

>IOMT_3MGSTAETQLTPVQVTDDEAALFAMQLASASVLPMALKSALELD
LLEIMAKNGSPMSPTEIASKLPTKNPEAPVMLDRILRLLTSYSVLTCSNR
KLSGDGVERIYGLGPVCKYLTKNEDGVSIAALCLMNQDKVLMESWYHLKD
AILDGGIPFNKAYGMSAFEYHGTDPRFNKVFNNGMSNHSTITMKKILETY
KGFEGLTSLVDVGGGIGATLKMIVSKYPNLKGINFDLPHVIEDAPSHPGI
EHVGGDMFVSVPKGDAIFMKWICHDWSDEHCVKFLKNCYESLPEDGKVIL
AECILPETPDSSLSTKQVVHVDCIMLAHNPGGKERTEKEFEALAKASGFK
GIKVVCDAFGVNLIELLKKL

Seq. ID No: 391

>IOMT_4MGSTAADMAASADEEACMYALQLVSSSILPMTLKNAIELGLLE
TLVAAGGKLLTPAEVAAKLPSTANPAAADMVDRMLRLLASYNVVSCTMEE
GKDGRLSRRYRAAPVCKFLTPNEDGVSMAALALMNQDKVLMESWYYLKDA
VLDGGIPFNKAYGMSAFEYHGTDPRFNRVFNEGMKNHSIIITKKLLEVYK
GFEGLGTIVDVGGGVGATVGAITAAYPAIKGINFDLPHVISEAQPFPGVT
HVGGDMFQKVPSGDAILMKWILHDWSDEHCATLLKNCYDALPAHGKVVLV
ECILPVNPEATPKAQGVFHVDMIMLAHNPGGRERYEREFEALAKGAGFKA
IKTTYIYANAFAIEFTK

Seq. ID No: 392

>IOMT_5MGSAGETQITPTHVNDEEANLFAMQLASASVLPMILKSALELD
LLEIIAKAGPNAQLSSSDIASQLPTKNPDAAVMLDRMMRLLACYNVLSSS
LRTLPDGKIERLYGLAPVAKYLVKTEDGVSIAPLSLMNQDKVLMESWYYL
TEAVLEGGIPFNKAHGMTSFEYHGKDARFNKVFNKGMADHSTITMKKILE
TYTGFEGLKSLVDVGGGTGAVISMIVSKYPSIKGFNFDLPHVIEEAPSYP
GVEHVGGDMFVSVPKADAVFMKWICHDWSDEHCVKFLKNCYDALPENGKV
IVAECILPVAPDSSLATKGVVHIDVIMLAHNPGGKERTEKEFEALAKGAG
FQGFRVCCSAFNSYIIEFLKKP

Seq. ID No: 393

>IOMT_6MGSTAETQITPVQVTDDEAALFAMQLASASVLPMVLKSALDLD
LLEIMAKNSSPMSPSEIASKLQTKNPEAPVMLDRILRLLTSYSILTCSNR
TILGGDSVERIYGLGPVCKYLTKNEDGVSIAALCLMNQDKVLMESWYHLK
DAVLDGGIPFNKAYGMSAFEYHGKDLRFNTVFNNGMSNHSTITMKKILET
YKGFEGLTSLVDVGGGIGATLKMIVSKYPDLKGINFDLPHVIEEATSHPG
IDHVGGDMFVSVPKGDAIFMKWICHDWSDEHCVKFLKNCYEALPEDGKVI
LAECILPETPDSSLSTKQVVHVDCIMLAHNPGGKERTEKEFEALAKGSGF
KGINVACNAFGVYVIELLKKM

Seq. ID No: 394

>IOMT_7MEMINFMHHMDSTWNLCGKDVVQAFDFSEFHTVYDLGGCSGGL
AKQFVSTYNDSTVTIMDLPKVVQTAKKYFVTDQEQQIHFIEGDLFNDPIP
EADLFIMARIIHDWTEEKCLELLRKIYQSCRPGGGVLLVEVLLNEDKSGP
LMSQLFSLNMLVQTEGRERTPSEYTKLLTDSGFRDIQVKITGKIYDA

Seq. ID No: 395

>IOMT_8MERLLDACVGLKLLKVELKSNKGYYSNTDVSTMYLVKSSPRTL
YYMIMFYSKTTYMCYNFLPQAVREGQCQYERAFGISSKDLFEALYRSEED
TLAFMYFMNSTWSICGKYVVQAFDLSEFHTIYDLGGCTGALAKQLVSTYK
ESTVTIMDMPNIVQAAKKHFVTDKEQQIHFLEGDFFNDPIPEA

Seq. ID No: 396

>IOMT_9MIPFNKAYGMTAFEYHGKDDRFNKVFNAGMFNHSTMTMKKILD
IYDGFNNLTTLVDVGGGTGASLNMIVSKHPSVKGINFDLPHVIQDATTYP
GIEHVGGDMFESVPKGDAIFMKWICHDWSDAHCLKFLKNCYKALPDNGKV
IVA

Seq. ID No: 397

>IOMT_10MAQAAAEAEGITPVMDLLFAAQGSSALLVCARLGLFDYISSQ
GEEGVSCKQLASRAQWSTRAASAVMVSLAASGILAVKPSSAGAQHCFEHS
YTLTPRAQRFLVTEKPGSMSAYTEIHWEASPELLLKKAAETEDEKRNFML
ETGGGAPSEVFLAAMQGQSSYAAMVLTRLVDLSDTRTFVDVGGGSGTFAI
EACKATPNLQGVVYDLAGACPTTDGFIARAGMAERVKTHAGNMFEDERFP
AADCYAFGNVLHDWSDQDNSKLLRKAFESLPAQGKVLLLEMLVEEDVVST
SPSAAGLNLCMVTNELGRQFKASELRAMLLEAGFAGAEVVSSPLTPYSLV
VGTKGEANPVASKPEAAAAAESESITPLMDVLFSAQHSAVLIVCSRLGVF
DFVGAQGESGASCAQVAAHAKWTTRAASAMLVSLACSGLLEPTPGSAAAQ
HCFEHSYRLTPLARRFLVAGQPGQLSAYTEIFWGASPKQLLEKASASLGE
WGEGNFMLDAEGGAPSEVFLAAMQAQSTYAAMVLTRLVDLSDVRTFVDVG
GGSGTLAIEACRAAPGLQGVVYDLAGACPVTDGFIARAGMAERVKTHAGN
MFADERFPAADCYAFGNVLHDWSDQDDGKLLRKAFESLPANGKVLLLEML
LAEDVESSTRSATGLNIVMVTNEQGRQFKGSELEAMLRAAGFAATEVVRS
PLTPYALVVGTKG

Seq. ID No: 398

>IOMT_11MSRTSWDEGEDVDLDSVAYGFMASQALFTGLELGIFDHIAAA
GAGGLSAAGIGKACGIEAPRVQTLLTSLVAVKCLKRDASAMYTLSPNTAQ
YMVTSSRHFYGDYLRYQIGRQFYHRMGALPEVMTSGKAPSYASWFSDPEV
ARTYTQAQHNGSVATAKYLIKKKLQLGGISAMLDVGGGSGAFSYVFTQAT
PGLHSKVLELPEVCRTGEGIREKQPEDVRSRVSFVELDASSPTWPVDDSA
FDVVLMSYISGSVPEPIIGSLYANAMKALRPGGRLLVHDFMVNDSLDGPA
LGALWGLQHVTVNADGLGLCPKEVIARMGAAGFDTSKCEAMEMIHGMTKL
IVGHKG

Seq. ID No: 399

>IOMT_12MCSSKELDFPHILIDYQHGFLVSKTIFTACELGVFDLLHEVQ
EPVPAATIASRLSTSEDGMERLLDACVGLKLLKVYLKNNKGYYSNTDVST
IYLVKSSPKTLHYMMIYYSKITYMCWHFLPQAVREGKRQYERALGTTSND
LFEIVYRSEEEMTTFMHFMDSTWNLCGKDIVQAFDLSEFHTVYDLGGCSG
SLAKQLVSTYKESTVTIMDLPKVVQAAKKHFVTDKEQQIHFLEGDFFNDP
LPEADLFIVARIIHDWTEETCIKLLKKMYHSCRPGGGVVIVELLLNEDKS
GPVISQVYSLYMLVQAEGKERTPSEYTKLLTDSGFKDIKVKATEKLFGAI
LGRK

Seq. ID No: 400

>IOMT_13MCSQEGEGYSLLKEYANGFMVSQVLFAACELGVFELLAEALE
PLDSAAVSSHLGSSPQGTELLLNTCVSLKLLQADVRGGKAVYANTELAST
YLVRGSPRSQRDMLLYAGRTAYVCWRHLAEAVREGRNQYLKAFGIPSEEL
FSAIYRSEDERLQFMQGLQDVWRLEGATVLAAFDLSPFPLICDLGGGSGA
LAKACVSLYPGCRAIVFDIPGVVQIAKRHFSASEDERISFHEGDFFKDAL
PEADLYILARVLHDWTDAKCSHLLQRVYRACRTGGGILVIESLLDTDGRG
PLTTLLYSLNMLVQTEGRERTPAEYRALLGPAGFRDVRCRRTGGTYDAVL
ARK

Seq. ID No: 401

>IOMT_14MGYAAPQARQSDKQIFDIYFGFLHSYALLFADEVGLFDLLRC
EALTLDQVSMATSLPSRSSQALLSLCASLGLLEKRGERFALSALTEGFLV
REAETSFCGVLASARGQAAAFSYDFFKASLLKGESQLFGGRDLFDNNAQD
PEHCEIFTRAMHSKSKGPAQAWVEKIDLSAHACLLDVGGGSGVHAISALA
RWPNLNAVVFDLPPVCAIADTFIERYQMTARAQTHGGDIWYTDYPFADAH
FYSDIFHDWPLERCRFLARKSFDALPSGGRIILHEMLFNAQKTGPRNVAA
YNANMLLWTQGQQLSEPEAADLLQAAGFVEILAFPTGYGDWSLVTGVKP

Seq. ID No: 402

>IOMT_15MGSIDAQMAAVEEESCIYAMQLAYTVVLPMTLKNAIELGMLE
ILMGAGGKMLSASEVAAQLPSTTTNPDAPAMVDRMLHLLASYKVVSCEVE
EGTHSRRYGPAPVCKWFTSNKDGDGASLAAMLLLTNEKVLLESLNHLKDA
VLDGGHPFLKAHGMTVYEYNKTDARMKRVFSQAMNNYSTIINRKLVEMYM
GFHDIAFLVDVGGGVGTTIRAITSKYPHIKGINFDLPHVIADAPQCPGVQ
HVAGDMFRNVPSGDAIILKWMLHNWTDEHCTTLLRNCYDALPPHGKVFIV
ENILPLKPDATSRGQQTSLSDMIMLMHTPAGRERSQREFQELGKAAGFTG
FKTTYIYGNSWVIELTT

Seq. ID No: 403

>IOMT_16MSFDTQHALQPYWDLAVAPVQADGLAAALELGIFEVLATPHT
PAQLADVLSLHGPHTALLLELLWSMQVLERDGADADTDANALRYRCTATT
LQYFCRDAVAFCGDAWLYRLHALRHFATQLNTLVRDGGKVTPYSTASGVN
WAAAAQQQIGQEQRAVTMRAALCVMQRVAPFADGNTPLRLLDAGGGPGWV
AIALAQAHAGVHGCVFDWPETVAVAAANIAHAQLSDRLETLGGDLDSDDI
GGGYDLIWCSSVLHFVPDMAAALRKMQAALKPGGVLVCIQAEIAAAPGDA
ARVLPYYLPMRMLGRTVTRHGELAQLLRDTGWRQVEQYGASDFPMAPVQV
LIARA

Seq. ID No: 404

>IOMT_17MQLASASVLPMVLKSAIELDLLDIIAKAGPGAYLSPSEVASQ
LPTSNPDAPVMLDRILRLLASYSVLTYSLRTLPDGRVERLYGVGPVCKFL
TKNEDGVSIAALCLMNQDKVLMESWYYLKDAVLEGGIPFNKAHGMTSFEY
HGKDLRFNKVFNKGMSDHSTITMKKILETYKGFEDLTSLVDVGGGTGAVL
STIVSKYPSIRGINFDLPHVIEDAPSYPGVDHVGGDMFVSVPKGDAIFMK
WICHDWSDEHCLKFLKNCYEALPDNGKVIVAECILPVAPDTSLAAKGVIH
IDVIMLAHNPGGKERTEKEFEALAKGAGFQGFRVMCCAFNTYIMEFIKKL

Seq. ID No: 405

>IOMT_18MLNHTTMVIKKILECYKGFETLKQLVDVGGGLGVALNLITSK
YPHIKGINFDLPHVVQHAPSYPGVEHVGGDMFKSVPKADAIFMKWILHDW
SDEHCVKLLKNCYAAIPNDGNVIVVDAVLPKMPEVSTSMRCTSQLDVLML
TQNPGGKERTEEEFMALATKAGFKGIRYQECFVNTFWLMEFFK

Seq. ID No: 406

>IOMT_19MERKEEVALLKGQAEIWQHLFAFADSMALKCAVELRLADIIH
SHGVPITLSQIASAIDSPSPDIAYLSRIMRSLVYKKIFTEHHPSDGGETV
LYGPTHTSRWLLHDAELTLAPFVLMENNQWQLAPWHFLSQCVKEGGIAFK
KAHGFEMWDFAARNPEFNKIFNDAMACTTKILMGVLLAEYKDGFGSIGSL
VDVGGGTGEMIAEIIKQHPHIKGMN

Seq. ID No: 407

>IOMT_20MGSASGSAERTQMGEDEACSFAMTITSGSVPPMVLKAVIELD
VLEIIKRAGPGAHLSPAEIAAQLPTTNPGAAAMLDRMLRLLASYDVLSYS
LHTLPDGRVERLYGLAPVCQFLTNNEDGVTLSALSLMNQDKVLMESWYHL
KDAVLDGGIPFNKAYGMTAFEYHGTDPRFNKVFNNGMSNHSTITMKKLLE
NYKGFEGVSTLVDVGGGTGATLNMIISKHPTIKGINFDLPHVIEDAPTYP
GVEHIGGDMFVSVPKGDAIFMKWICHDWSDEHCLRFLKNCYAALADHGKV
IVCEYILPVAPETNHAARTVFHVDAIMLAHNPGGKERTEQEFESLAKGAG
FEGFRVAFFF

Seq. ID No: 408

>IOMT_21MALNPPHQNNVMEKEDLCSFALSIATSSSLSMVLKAIIELDI
IGIINRAGPGAHLSPAQIAAQLPTKDPGATASMLDRMLRVLANNSILSCS
LRALPNDGPIERLYGLAPVCQFFTKPEDFGPMVLFSQDKVYTDTWHHLKD
AVLDGGSAFKKAHGTTLFEYLGTDMRFSKVFNDAMSSSSTITMKKMLENY
NGFDGLSTLVDVGGGTGETLNMIIAKYPTIRGINFDLPHVINDAPNYDGV
EHVVGDMFVSVPKGDAIFMKWICHDWSDKLCLKLLKNCYTALPNHGKVIV
CECILPVAPETSHSARVASNLDMHMLAYCRGGKERTEQEFEALAKGAGFE
SFRVVCSAYDLKLYMC

Seq. ID No: 409

>IOMT_22MAEIPTSSNPSDDPETQKLNGNEEDYDHHHDEDPESDDENYE
YALQIAEMLPFPMVMHTAIELDLLGIIATAGPDRQLSAAEIAAALPAAGN
PDAPAMLDRMLYLLATYSVVTCTAVDGGASGGVVRKYGLAPVAKYFVSNK
DGVSLGAVISLNQDQAVLASWSKLKEAVLEGGIPFNKVHGMDAFEYQGTN
PRFNEIFNKAMYDQSTYIIKKIVRRYKGFENIQRLVDVGGGLGHTLRVIT
SNYPSIKGINFDLPHVIQHAPTIPGVEHVGGDMFESIPHGDAIFMKCILH
DWSDEHCLKTLKNCYKALPRKGKVIVVQMNMIEEPQTTPLAKAISQMDLW
MMTQNPGGKERTRREFQALAEAAGFAEFNPVCHVAGFWVMEFLK

Seq. ID No: 410

>IOMT_23MSPIDLANELQTLVTSTYSGDVTDPFKLYKAKHSISDLCLSL
LRAVQGPEEYTAILAESCQESSALNVVASLGVADHIAESPNGELTLQELS
EKVKADEKYLSVVLSSLVYHGYFKEVGGFGSQVYANNDFSSLLLSEETNA
KGGKSMKDAIGLSADDGAKATTRLLDAATGKAKGEAKTAANIAFDFSESL
FQWMASPGNEWRGKRTAKAMVQLHGMANGGIGEDYPWEKLATPIIDIGGG
IGSFQGMLLALPKNKELTFTIFDIEKTVEHAKKVWAGKPQWMQDKVSFIA
GDFMKSSPNDSKIPTPAQGAGTYVIRHVLHDWDDAQVVTILKHVRNAMLG
SPASTPPKLLLVEMMLNETSSRFTRTTSLQLLSLNGGITRTEVQFRRLIK
EAGFTVDSVTEVRGVDLVVELSPASL

Seq. ID No: 411

>IOMT_24MPSTTISQLVGLIQQSVMALEKLCLENRTSLPDLDAFHFDQS
SETFRSLPGAAQDAKIAVAACMQLIAILSPPTDTVYRAALGGHLSFATRT
CLEANITEILREAGPEGLHINDIASKCGLDPSKLGRVIRYLVIHHIYREV
KPDVFTNNRTSSTMDTGKPLDKLISEPDRKYDDTGFPALISHFMDVDQKC
GAVGWDVLKDPVLGHSCDLTETIFSRAFNTKSKYWDFFDHPENHYMRRRF
DYAMKGLGAIEDHDMVLHAFSWEDLDKGSVIVDVGGGIGTAMLPLARKYP
NFDIVIQDLPIVIEEGTKFWSQNLPDAVANGNIKLHAHNFFDEQPIKNAS
VFYLRHVLHDWPMPDMVKILRRLRDVAAANTTLIILDYILPYSCKMFADK
DAVSIASARYYSEAPEPLLPNYTHKNVISDSDMYVFQMMFHYNSQEHTYL
SLKSLLDASGWRLVRLRAIDPRNDYFQSIECKILA

Seq. ID No: 412

>IOMT_25MAQPMMLALAKLISDSVAKVDQLCIEQGVIFPSLDDPFTTES
ESIKLHPDVAEASNYIISAAAQLIAILRPVPVTLSTSAIHVHVSSALRVV
VDSNVVEILREAGPQGLHVKKISEKNGVEAGKLGRLLRLLASGHMFKEIT
PDVFATNRISSALDTGKPYEELVKNPGEKLIGTNGIAAYISRSTDESVKS
SGFLYEALTYSSSEKVPLPPSPFNLAFNTELHIFSWLAQKGNEHRLQRFG
IAFDGFDKMLPVNGVTKGYRWGSLPKGSIVVDVGGGVGSESMKIAKTFPD
LKVIIQDAEGVVANGVKFYETRFPEGLSSGQVTFQAHDFFTPNPVTNARV
FFMRFVLHDWPDATCVKILKNLRAAAAPDTELIINECLIQYACSTESEIS
KSIPGGRFKPPPSPLLPNLGYARIFHYLIDLQMAIVAHGVERTVEQYASI
LQKSGWKLKEVLRMPESAYSLHKLVAVPQPE

Seq. ID No: 413

>IOMT_26MTRLTDSLGMLRSKLVPPQATMLQLLTGYRVSQGIYVVAKLG
IADLLATGSKTSQDLAAITNVHAPSLYRLMRSLASLGIFTETENGRFELT
PLAATLRSDHPNSVHDAAIMFLEDWHWQAWGNFFDCVKTGETALEKTFGT
SNVFDYFETQNPEAGQHFDNAMTNTSVMTNQALPTAYNFGAFKTLVDVGG
GQGSFLSALFHQWDHLHGILFDLPPVIESAEQQNLLSGFEKRTTLAAGDF
FKAVPDGADAYLLKTIIHDWDDASAIAILKTCRRAMNHDSKLLLVELIVP
SGNAPSLSKILDLEMLAVFGGVERTEAEYRSLLLSAGLKLTRIYDSPCPW
SVIEAIPV

Seq. ID No: 414

>IOMT_27MSMPPAHSRLYSRSFLSMLPDAITPFPYLPPDATDTRPLLAE
LEALLEIINSSARLAITEYKKHGNNVPTIYSTEFHPLDFATDTVALKKAI
RLLEDACQQLCASLAPPQHTLANVSRVHHRQYVTQLTTHDILEKYPSGSH
IRELSQTVGLEKGKLARILRVFAFKGCFIEVDTDVFASNRLSLIMKSSND
CGCLTCIHAQDVSQGAGVLYETLTEPEYAMSYEPDKAPMIYVLKRKGLKG
SFFDWMKADAKRRENYHYAMIALGPVMGSLSILHHYPWNDVATVCDVGAS
VGSVSIPLSKAHPHLKITDQDLPEVLEAARSVWEKEAFEALREKRVEFLT
LDFFKEAPVPGKDVYYLRHIIHDWPDAEAAVILRNISKAMEPHSRLLIHN
YVIAGANRRPDEEQRAPEPMLPNFGAGDSRKYRQDLNMWILHNAKERTVD
DQITLA

Seq. ID No: 415

>IOMT_28MAPGREGELDRDFRVLMSLAHGFMVSQVLFAALDLGIFDLAA
QGPVAAEAVAQTGGWSPRGTQLLMDACTRLGLLRGAGDGSYTNSALSSTF
LVSGSPQSQRCMLLYLAGTTYGCWAHLAAGVREGRNQYSRAVGISAEDPF
SAIYRSEPERLLFMRGLQETWSLCGGRVLTAFDLSRFRVICDLGGGSGAL
AQEAARLYPGSSVCVFDLPDVIAAARTHFLSPGARPSVRFVAGDFFRSRL
PRADLFILARVLHDWADGACVELLGRLHRACRPGGALLLVEAVLAKGGAG
PLRSLLLSLNMMLQAEGWERQASDYRNLATRAGFPRLQLRRPGGPYHAML
ARRGPRPGIITGVGSNTTGTGSFVTGIRRDVPGARSDAAGTGSGTGNTGS
GIMLQGETLESEVSAPQAGSDVGGAGNEPRSGTLKQGDWK

Seq. ID No: 416

>IOMT_29MEVVPSWFKETLDKSQFSAPYEYAVETAKQKALEVARRMHVK
HLKTPDIVIGADTIVTLEGAILEKPFDKQDAYNMLSRLSGKEHSVFTGVV
IVHCRSKEENHLETDIIDFYEETKVKFADLSEDLLWEYIDSGEPMDKAGG
YGIQSLGGMLVESVHGDFLNVVGFPLNHFCRKLTEIYYPPPKQAICRVKH
DSIPYVESFENLSDVETDCTSTSKACEAKKAVQDGVCKADGSGSAVLQNG
IEERPVHCAQQLSKITQLLDGFKASQTLFAASKLKVFDKLKDKGALKAME
IAEKINASVHGTERLLDACVALGLLEKTHQVYSNTELANTYLVSDGAFSI
HEYITYSSDHLWSHFTHLDSAVVEGGGQHQTAVKKACDNRNGSEVKERFM
RAMHCMLKITARDLVTAFDLSKYSSACDLGGCTGALAHELVWTYPEMKVN
VFDLPEVIKHTSQFQPESFDSSRVTFSSGNFMEDTLPEADLYILSRVLHD
LPEGKLNHLLKKVSEACCPGRSALLVAEIVLDEDKKESRGLLQSLSMGEG
KQRSGTEYKKLLENHGFNSVQIKSTGNLLDAILAIKTS

Seq. ID No: 417

>IOMT_30MDTVKNLQASNVPSSLSQEDEEVFTSGLHVCSSEVFSHALSN
CIQLGLFDIIAEAGPSAYLTATEITAQLPTKNPDAVSMIDRMLRLFSCHS
LLNSSLKTVADDVVETRYGLSPIGHL FVRKKDGVTMAACFTDYKAWTEA
WLHLKDAILEGGNPYEKAHGVPIYEHISSDTESVKGFSQAMDSISSFIMK
KVLENYSGFKGLGSLVDVGGGSGFALNMITSEYPSISCINFDLPHVVQEA
PYHPGVKHVGGDMFLDIPSADAIMIKEVLHNWGNEDCVKVLKNCYEALPK
GGKVIVVSHVMPEVVGSSNAAAKYVCQLDVMMLLFGGGKERTEKEFKALG
KAAGFSGFQLICFAAYNAVAVMEFYK

Seq. ID No: 418

>IOMT_31MAEDVAAVADEEACMYAMQLASSSILPMTLKNALELGLLEVL
QKDAGKALAAEEVVARLPVAPTNPAAADMVDRMLRLLASYDVVKCQMEDK
DGKYERRYSAAPVGKWLTPNEDGVSMAALALMNQDKVLMESWYYLKDAVL
DGGIPFNKAYGMTAFEYHGTDPRFNRVFNEGMKNHSVIITKKLLEFYTGF
DESVSTLVDVGGGIGATLHAITSHHSHIRGVNFDLPHVISEAPPFPGVQH
VGGDMFKSVPAGDAILMKWILHDWSDAHCATLLKNCYDALPEKGGKVIVV
ECVLPVTTDAVPKAQGVFHVDMIMLAHNPGGRERYEREFRDLAKAAGFSG
FKATYIYANAWAIEFIK

Seq. ID No: 419

>IOMT_32MTSLQDLDYPQQLLEYKDGFLVSKTMFTACELGIFDLLHKSD
EALSALTISSHLGTSADGTDRLLSACVGLKLLKVEMKNNEAFFSNTDVSD
VYLVQLSPRSLYHMMMYYSQTLYKCWHFLPDAIREGKSQYERAFGVSSGD
IFKALYRSEEEMVTFMHHMDSVWNICGKDIIAAFDLSSFNEVCDLGGCSG
GLAKQLLSIYPSSSVTILDLPEVVQTAKKHFITDADCNIAFLQGNFFNDP
IPEADLYIMARIIHDWTQEKCLQLLNKIYKSCRPGGGVLLVEVLLNEDRS
GPLTSQLYSLNMLVQTEGRERSPCEYTKLLAHSGFRDIQVKATGKIYDAI
LGRK

Seq. ID No: 420

>MUP1MSEGRTFLSQLNVFNKENYQFSSSTTKKEVSNSTVDADNGASDFE
AGQQFATELDQGEKQLGILSCIGLICNRMLGTGVFAVSSTIYTLCGSVGL
ALIMWAVGAIIAISGLYVYMEFGTAIPKNGGEKNYLEAIFRKPKFFITCM
YAAYIFFLGWAAGNSINTAIMFLTAADTEVTKWNQRGIGVAVVFFAFLIN
SLNVKIGLYLQNILGIFKIGIVLFISITGWVALGGGLKDGYQSHNFRNAF
EGTETATAYGIVNALYSVIWSFVGYSNVNYALGEVKNPVRTLKIAGPTSM
VFLAIIYIFVNIAYFAVVPKDKLISSKLILAADFFDIVFGGQAKRAAAAL
VGLSALGNVLSVIFSQGRIIQQLGREGVLPFSNFFASSKPFNSPMVGLFQ
HFIVCTVTILAPPPGDAYLLVQNLISYPMNIINFAISAGLLWIYWQRRQG
KIEWNPPIKAGVFVTGFFTLSNLYLIIAPYVPPSNGESVYSSMPYWIHCV
IAWGIFFFGGVYYVVWAQLLPRWGHYKLVSKDVLGEDGFWRVKIAKVYDD
TIGDVDTQEDGVIETNIIEHYKSEQEKSL

Seq. ID No: 421

>NAT_1MAPIEEEEPLPEELVLLERTLADGSTEQIIFSSAGDVNVYDLQA
LCDKVGWPRRPLTKIAASLRNSYLVATLHSVTMPSKAEGEERKQLIGMAR
ATSDHAFNATIWDVLVDPSYQGQGLGKALMEKVIRTLLQRDISNITLFAD
NKVVDFYKNLGFEADPQGIKGMFWYPRF

Seq. ID No: 422

>NAT_2MSTPSVHCLKPSPLHLPSGIPGSPGRQRRHTLPANEFRCLTPED
AAGVFEIEREAFISVSGNCPLNLDEVQHFLTLCPELSLGWFVEGRLVAFI
IGSLWDEERLTQESLALHRPRGHSAHLHALAVHRSFRQQGKGSVLLWRYL
HHVGAQPAVRRAVLMCEDALVPFYQRFGFHPAGPCAIVVGSLTFTEMHCS
LRGHAALRRNSDR

Seq. ID No: 423

>NAT_3MTSDVGADEHATTEAGGGRLQAGGHSSAEEASERCPPAAAPPSG
MKGAADCGPQDSSARDDVSFIPYKDETDMPGIVELIEKDLSEPYSIFTYR
YFINNWPELCFLTMRGDSCVGAIVCKLDVHRCRNTNRGYIAMLAVEKGLR
GKGIGSTLVRLCLDKMREMGADECVLETEVTNKGALGLYRNMGFVKEKRL
HKYYLNGNDAFRLKFLFKLPEGFDRGEGCLGPLCEVPPVTT

Seq. ID No: 424

>NAT_4MVSIRPATVDDLLAMQACNLCCLPENYQMKYYFYHMLSWPQLLY
VAEDYGKKIVGYVLAKMEEDSSEVHGHITSLAVLRSHRKLGLASKLMRAA
MAAMEETFGAEHVSLHVRVTNRAAFTLYSETLGFEINDVEHKYYADKEDA
YDMRKMFETGLKKQEAGKQKKKEKEKEKEKEKEKEKEKEKEKEKGKDSQP
VEQQGGAAGADKEAQRSKRARSGDRKRNGRPRRRSGSG

Seq. ID No: 425

>NAT_5MLPRPPVGAAKEGHLTLFYRELRWLCPGTRFYFVVRDPAENVRS
IADRLALGPEGLRRPPRIVARADLGWREVLNMSYAGVREESALGTLVGRW
NLMARLYLDAPKGAMALVRYEDLVAEATWEAEVRRVAAAETLDLRERVLW
PGRPDLCTLPGDESALHFGAVAAGKVLGVISVFLSPEPGGRAQFRKFAVD
PEVQGRGLGRRLLEQAVAAAREAGAGSLFCHARADQQGFYERRGLHVVGE
PFEKYGGKPYVEMEVPFQ

Seq. ID No: 426

>NAT_6MKGSRIELGDVTPHNIKQLKRLNQVIFPVSYNDKFYKDVLEVGE
LAKLAYFNDIAVGAVCCRVDHSQNQKRLYIMTLGCLAPYRRLGIGTKMLN
HVLNICEKDGTFDNIYLHVQISNESAIDFYRKFGFEIIETKKNYYKRIEP
ADAHVLQKNLKVSSPAPNADVQKSEN

Seq. ID No: 427

>NAT_7MSTPSIHCLKPSPLHLPSGIPGSPGRQRRHTLPANEFRCLTPED
AAGVFEIEREAFISVSGNCPLNLDEVRHFLTLCPELSLGWFVEGRLVAFI
IGSLWDEERLTQESLTLHRPGGRTAHLHALAVHHSFRQQGKGSVLLWRYL
QHAGGQPAVRRAVLMCEDALVPFYQRFGFHPAGPCAVVVGSLTFTEMHCS
LRGHAALRRNSDR

Seq. ID No: 428

>NAT_8MSSGGVIVDLHRNSTNWAKVVDDIVKLERKIFPKHESLARSFDE
ELGKKNTGLIYMEVDGEVVGYAMYSWPSSMYACVTKLAVKENCRRQGHGE
ALLKAAIKKCRTRNVHRISLHVDPLRNPAISLYKKFGFQVDNLIDGYYSS
DRNAYRMYLDFDAD

Seq. ID No: 429

>NAT_9MDERVVVELKKSLADYPKVLEELVRIEKKVFPKHESLSRSFDEE
LGKKNSGLLYICSNGEVAGYVMYSWPSALLAVITKLAVKEKYRRQGYGEA
LLRAAIQKCKTRNIQRISLHVDPSRTPAANLYKKLGFRIDSLVEKYYAAD
RDAYRMYLDFDADV

Seq. ID No: 430

>NAT_10MMEGAQEDEETEEKAEFDASEIEYVSYGGEHHLPLIMCLVDHE
LSEPYSIFTYRYFVYLWPQLCFLAFHKGRCVGTVVCKMGDHRHTFRGYIA
MLVVIKPYRGRGIATELVTRAIKVMMESGCDEVTLEAEVTNNGALALYGR
LGFIRAKRLFRYYLNGVDAFRLKLLFPRSEMHPSLHLLADQDGHDDQIAM
EGEA

Seq. ID No: 431

>NAT_11MKQVGISLDAVREKNLMQLKKLNVVLFPVRYNDKYYADALASG
EFTKLAYYSDICVGAIACRLEKKDPGAVRVYIMTLGVLAPYRGLGIGTEL
LNHVLEQCSKQNISEIYLHVQTNNDDAINFYKKFGFEVTETIQNYYTNIT
PPDCYVVSKRLEAQPKK

Seq. ID No: 432

>NAT_12MNIRVAKVEDLMGMQACNLQNLPENYMMKFWMYHSMTWPQISF
VAEDHKGRIVGYVLAKIEDPSEEGTTEEIHGHVNSISVLRSYRRLGLAKK
LMLLSQEAMSSIYKASYVSLHVRKSNKAAIALYKDTLGFEVAKVEKKYYG
DGEDALSMRLSLKNP

Seq. ID No: 433

>NAT_13MSDFQVAPLTARELARVRDLHAKLLPVQYPVSFFIHLLVIPSR
ACYVAYSHGSPVGFISAALHNPTRCFISGDSEVSPRLEILTLGVLPAFQH
RGLARRLIMSLVNAFKQDPATPILIYANVSTTNTRALQFYERMGILVSSD
IITNLYRTLSYGSRDAYLVVGAL

Seq. ID No: 434

>NAT_14MLSIHPLKPEALHLPLGTSEFLGCQRRHTLPASEFRCLTPEDA
TSAFEIEREAFISVSGTCPLHLDEIRHFLTLCPELSLGWFEEGCLVAFII
GSLWDKERLTQESLTLHRPGGRTAHLHVLAVHRTFRQQGKGSVLLWRYLH
HLGSQPAVRRAVLMCENALVPFYEKFGFQAMGPCAITMGSLTFTELQCSL
RCHTFLRRNSGC

Seq. ID No: 435

>NAT_15MADAPSGPSVLSHYPGAGLALPPGDEQEDGEEEEEGRYEPRRG
HHHHRRHHQQQQLNGLISPDLRHIKALKSKLPPPPHDERTGAPNGLERLQ
DLEEEEAVLASRMGACSLHPGDGSIRYVRYESELQMPDIMRLITKDLSEP
YSIYTYRYFIHNWPQLCFLAMVEEECVGAIVCKLDMHKKMFRRGYIAMLA
VDSKYRRKGIGTNLVKKAIYAMVEGDCDEVVLETEITNKSALKLYENLGF
VRDKRLFRYYLNGVDALRLKLWLR

Seq. ID No: 436

>NAT_16MDAAMPTEISFRQPTPDDAARCFEIETSAYEGDEAATLEKIAT
RIALYPEGFVILEADGKIAGFINSGCAFEVVMSDEEFKELVGHDPAAPNA
VIMSVVVDPAEQGKGYSKLLMQHFIARMKAMDKKTIHLMCKEAHVPLYAR
MGYRYTRPSASDHGGMAWHEMVMEL

Seq. ID No: 437

>NAT_17MEGLHSEWEVGAELKALGAVPKPFIGSHVSGKLIQRLKQDLRQ
SWDRGQSQARPTCTLPQPLPAPLGSSVPSASAQTQVSRLVPVAPPQPDPA
MSVLNAVPFMRPIHLRSPRQQRRHTLPASEFRCLSPEDAVSVFEIEREAF
ISVSGDCPLHLNEVRHFLTLCPELSLGWFEEGRLVAFIIGSLWNQDRLSQ
DALTLHKAEGSSVHIHVLAVHRTFRQQGKGSILLWRYLQYLRCLPFARRA
VLMCEDFLVPFYSKCGFKAVGPCDITVGPLTFIEMQCPVQGHAFMRRNSG
C

Seq. ID No: 438

>PSIK_1MQANRPISDQDQDQFKLNLTTADGTRSYLEKHLSLNVEAVERL
SGGFINFVWRAKLGTPYEGQNSIVVKHAPPFTAMDSSLNVAVERLKFEYD
SLKMIGSEPSIAGEDALISVPSVYHHDNIKHVLIMQDVGTMSTLRDFMGA
SPPPPTDMAALIGCQLATFIAGLHNWGRNNESARAGLSANAYGRTVMDLC
GYQTVVPNATASGILDPLLSTAMAALAERDKTSEETAIMGDFWALNVLVD
IDMSASGEKALKNIWIVDWEACRYGSPAVDVATFAGDCYLISRIHNETAT
DAMRRNFLGTYVALAKVDPMEVVIGMGTMWIMWTKYQEDIGEAEKRERVA
KGVEYIHKGWERSREWLPVSLAQELIA

Seq. ID No: 439

>PSIK_2MDLTTGDGVRVYLTAHMTLKVESTERLSGGYCNFVWRAKLKTP
YEGQNSVIVKYAAPFTSWDQTIELGVERLAFECMSLKMITSETPLLEENG
LVAVPTVYHYDSTANVLVMQDIGSIATLHGFLRSNTPPTVPMAALIGAKL
AAFIAGVHNWGRNNLPAHTRLSANTVGRTAMKKLCYETIVPKAAKSGVVD
PLLPMVVAALSEEAMTNDETLVMGDFWTANVLIDVQESHTGEQVLKKLWV
IDWESCRYGNPATDIASFAGDSYLVSRFQDHGLGEALRHSFLETYAALAK
VDPLRVALGLGAHWIMWTDDLGQGGEAETRECVDKGLEYIQRAWDQSAEW
VSLSLAKELVVL

Seq. ID No: 440

>PSIK_3MANENPDLLTVAGVLRFLAPTPFASDEVHPLSGGNCNFVYRIH
LRTPYNNISTLVLKHAEPYVAASAHRMPLAVERQNTEVTAMNAVKAILSS
DAVVIVPTIHHFDDVAHVVIMDDCGVGAVTLKQLMLKNPPPVSVAKALGA
GLGEFLSRLHVWGRDPQTSNHVSFDQNQQGRTISGYVTYGRLVSTLTGKD
NIPALSDPPLDIAQSKLDTISALSSEKIHAINTSHQTLTMGDFWPGNIMV
RLNPAGDSLERAYVLDWEVAKPGVAGLDIGQFCAEMHSLRRFSPACDASA
TTVLDAFLKTYRDAAGVDVGVAKDAMVHVGAHLVAWTPRVPWGSKERTRE
VVEEGVGYLVEGYAATQEWLRGSLVGRLV

Seq. ID No: 441

>PSIK_4MEIEWCDLDLTSESRPPTHKYTYFATALMPFDLTTRDGVRMYL
TAYLALDVMSVERLSGGYCNFSWRAKLESPYEGQISIVVKHAAPFTSWDR
NTELGVERLAFEYKALKILNSEPSVIAKNSLVAVPAVYHYDPTANALIMQ
DVGSIPTLHALLRNNALPPVPMAEKISNELAAFIAGIHNWGRNNQEARAN
LSQNLVGRTAIRKLCYETLVPKAEKSGVDDPLLQQVAAALSEEVMNSEET
LVMGDFWTANVMVDIQETGAGVRSLRKIWVIDWEGCRYGSPAADIASFAG
DSYLVARFHHHDLGETLRHSFLETYAGLAKVDPFRVALGSGAHWIMWTDD
LSEQEEGEIRECVDKGVEYIHRAWEQSTKWISLSLAKELVT

Seq. ID No: 442

>PSIM_1MHFSYDFTMCNPPFYGDYAELTRLRESKLKGPFGGAHEGVSTE
LFTAGGEIHEFIASELNQGKTLRWVVGWTFHKDLFDKKVIEPLHILIKLK
PLSCNTLELLDSDHELATPYSKKRCLRRISFTPKGRPPTKQTISVMHPNE
ASWASLLNHLQALDISVTVGNHFLTAEVKDPTWTRAWRRSSKVTSKITPF
SFSGQFSDPPERLLVLQLLVDESQTSEDILLSFQSL

Seq. ID No: 443

>PSIM_2MHPRNPYRTPPDYAALARSFPELKPYVSRNANGTVSVDYQDEA
ALRCLTRALLYRDFGLSVDLPKDRLCPTVPNRLNYILWIEDILNVSSLSR
LQSNSEATVRGLDIGTGASAIYPLLGCRVSPRWDFYATDIDAQSLAHARA
NITRNGLQGRINLVAADPKGSIFGPLESKHDTTFEFTMCNPPFYSSEEDI
AQSAAVKKLASNAVCTGAAVEMITPGGEAAFVVRMVRESLALKMRCQWYT
SMLGKMSSLTEIVGLLRENSIDNYAITEFVQGKTRRWAIAWSFGHVRLLD
SLARLSGGPLQSLMPTRNTCRRSFAVPRMVLHKHLILVLDGIEGTSQTPM
SIPVGAGDADGLYGLQISASRDTWSRAARRKRQHGAMDISLDNDEVGMKC
LIKVLSVEEAREGAEAVVLECTWVYGHERALFESFWGHVCRKIGEANG

Seq. ID No: 444

>PSIM_3MHARSIFNPNSAQFQARLTFSELSNEFPKLKPFLKYKRSRKQN
EADPLSSQCTFIDFKDPVATRAYNEVLLKKYFDLSLEFLPGSLCPAVPNR
LNYTLWLEDVLNVFPGSMGANNQRDELRGLDIGTGSSCIYPLLICRTHPN
WRMAGSDINPSSIEIAKKNVQENRLLDRIQLFLTTDKRDSVLEGQIFQTH
LFFNSKKCLQDEKPARFCYDFTMCNPPFYSDVEDLNNSRQAKTTTILGGG
HEGVSSELFTTGGELLFLSQMVEESFLYKDKVGLVSSYFFVLKCIVEILR
LLGQHKIQETIASKLIQGKTIRWVIGWTFHKDLFDLKHPSCNTLKVQDIH
CTNEEAPASKKICLGKIPLNSKEHIPSKQLISSSQSSDIGWTRLKSRLGD
LRIEFSLEKLSLIGKVVYPTWTRAWRRNGKAKSKPTPFSFSAQQSDASET
TIQLELLASEIEEPDENTLVSFQSLCNHLRSYLKD

Seq. ID No: 445

>PSIM_4MSDIDAQSLVYARANVARNALEGRIAVVTAEPEGSIFGPIEAE
KEIQFDFTMCNPPFYASAEDIAQSAATKELGPNAVCTGAAVEMITSGGEG
AFVARMIDNYAITEFVQGQTRRWAIAWSFGHDRLPDSLARLSSGPLQSLM
PTRNTCRRSFTFARMNLLSRLEQVLNNIEGLSHSNMSPSEDRGSGGRPSS
LLVSVARDTWSRAARRKKQRGSMDTSLDNDTSGLICSVKVLFDEEGREGS
EIASLECTWIHGRERALFESFWGHICRKVGEVSG

Seq. ID No: 446

>PSIM_5EGLSPFLLMHPRNQYCKKKPDFADLAKSHPPLREHLKWKTEDY
ATIDFKSPSAQKELTRALLKQDFHLDVDMPVDKLVPTVPQKLNYIHWIED
LLSGGRSDSIPRGEGIRGIDIGTGPACIYPLLATSLNKWTFVATDIDAVS
LEYAVKNVSRNDMEGRIRVKGVDPDTLLVGVVRDEQFDFCMCNPPFYGID
EDHHDNQRPPPPYSSCSAQAHEVRVQGGEVGFVSRMVEESLLLPSRVRWF
TSMVGKKGSLKSLRALLRKREVPTVTTTEFVQGVTKRWAVAWSFTEQVPC
IPSHSLPCTVPLLGSTSSAEGRAYAEQWLERVLNHMEVTFTKKDQDGYTC
TAERATWANQRRKRRLMQRPMMSPEAAAKRSCGGSDNTSEGVPRNDSDTL
VSAGHLSPKADSLERNASSDLAAQLSALTPPYHVTFWCGVQPSVPPSTNK
AELELKMVLIDGGSGTQPLQPIAQYMKNNWSATDSRPTSDRSSQ

Seq. ID No: 447

>PSIM_6CIYPLLGATMNGWYFLATEVDDICFDYATKNVEQNNLSDLIKV
VKVPQKTLLMDALKEETEIVYDFCMCNPPFFANQLEAKGVNSRNSRRPPP
SSVNTGGVTEIMAEGGELEFVKRIIHDSLQLKKRLRWYSCMLGKKCSLAP
LKEELRKQGVPKVTHTEFCQGRTMRWALAWSFYDDVIVPSPPNKKRKLER
ARKPLSFTLPEAGLKELQSKALALGGTACSPVDRVAALLEKTLTDLRVLH
KRVPCRKQEQSLFLTAVENTWIHGRQKRREQSRQLRELPRAPPCAGTSSQ
TTVATADSVKTPASQTQSASTQNSNSQDDSSQNKRASAQELAGQQPTDKA
GSSASSDEISIKVLHNSTGEQKEVTENLSSEAVDMEFSTSTEAVQETGSK
EAPSAESEPPSKRPLSPGTVEQFLFKCLLNVMLEESDVMIEMHWVEGQNK
DLMNQLCTYLKNTLLKSVAKS

Seq. ID No: 448

>PSIM_7MGSKKRRRRREERPTIHPKNKYSENPPDFALLASLYPSFEPFV
FYSRDGRPRIDWTDFNATRELTRVLLLHDHGLNWWIPDGQLCPTVPNRSN
YIHWIEDLLSSNIIPTTSRNGDKVKGFDIGTGANCIYPLLGASLLGWSFV
GSDMTDVALEWAEKNVKSNPHISELIEIRKVDNSESTPSIQESLTGKSVQ
DESNMDMSGHMDEEAEPSSSSSFNLPAGAQSSYHGPPVLVGVVRDGEQFD
FCICNPPFFESMEEAGLNPKTSCGGTPEEMVCSGGERAFITRIIEDSVAL
KQTFRWYTSMVGRKSNLKFLISKLRKVGVTIVKTTEFVQGQTCRWGLAWS
FVPPARKIISPHVAEKKNLSFMLECTLINRSLYQMINVTQS

Seq. ID No: 449

>PSIM_8MHPRNPYRQLLDFASLAEAYEPLKPHLKPTRSPTAGGLSYTID
FKNSESQRQLTKAILYRDFGLRIALPDHRLCPPVPNSRLNYILWLQDIIK
AHDEYMDRPASCICGLDIGTGASAIYLLLGCRVEPSFRFIGTELDDISFS
YATQNVESNGLSDRIHLIKTTSNDPILLPFDLNPAWSCDFTMCNPPFYES
EEEMARSAQAKELAPNAVCTGAQVEMVTPGGELAFVSQIVKESLKYTTRC
RWYTSMLGKLSSLTKLVGLLREYAISNYAITEFVQGQTRRWAVAWSFGET
HLPDSVARISNPTLQPLLPERNTSRHVINISLPPFSTRTVKSKQSIKALS
EVLSQIKDVTVQRLYQVEHLEPTEEEEEDKSLYRLLVYAKQNMWSRSARR
QRGRETGHKANDKGCAVGGPLTSIPATLDGLLCGIEIKAPLIKQEQQDVE
MEFVFQWVHGQDRSMFESFVNHVTRKMKCNIVLD

Seq. ID No: 450

>PSIM_9MALNKSMHPRNRYKDKPPDFAFLASKYPEFKQHVDVGLSGKVG
LNFKDPGAVRALTCTLLKEDFGLTIDIPLERLIPTVPLRLNYIHWVEDLI
NFHDSDKTTVRRGIDIGTGASCIYPLLGATLNGWYFLATEVDDICYNYAK
KNVEQNHLADLIKVVKVPQKTLLMDALKEESGIIYDFCMCNPPFFANQME
AQGVNSRNPRRPPPSSVNTGGITEIMAEGGELEFVKRIIHDSLQLKKRLR
WYSCMLGKKCSLAPLKEELRIQGVPKVAHTEFYQGRTMRWALAWSFYDDV
TIPNPPSKKRKLEKPRKPMMFSVLETTVKMLMDKFDCSVDSEHVSVVTDC
LKKILTDLKVQHKPVPCGNGEESLFLTAIENSWVHIRRKKRDRMRQLREL
PRAPDENFLLVQKDERQAEDEETTEKTVSSSEKSVSTSGIDEAAALPPNP
EDSISESMGEDSRQLPEEVKDTSALGQITDVDEHQNTMEASQPCSSNSAF
LFKCLVNVKKEATNVLVEMHWVEGHNRDLMNQLCTYLRNQICKIATS

Seq. ID No: 451

>PsiHchimera_1MFCRGLLSLMAIIIVYFIAQKRRRARLPPGPRGLPL
IGNLHQAPKEAVWLTFHKWVKEYGNLVSVNFGGTEMVILNTLETITDLLE
KRGSIYSGRLESTMVNELMGWEFDLGFITYGDRWREERRMFAKEFSEKGI
KQFRHAQVKAAHQLVQQLTKTPDRWAQHIRHQIAAMSLDIGYGIDLAEDD
PWLEATHLANEGLAIASVPGKFWVDSFPSLKYLPAWFPGAVFKRKAKVWR
EAADHMVDMPYETMRKLAPQGLTRPSYASARLQAMDLNGDLEHQEHVIKN
TAAEVNVGGGDTTVSAMSAFILAMVKYPEVQRKVQAELDALTNNGQIPDY
DEEDDSLPYLTACIKELFRWNQIAPLAIPHKLMKDDVYRGYLIPKNTLVF
ANTWAVLNDPEVYPDPSVFRPERYLGPDGKPDNTVRDPRKAAFGYGRRNC
PGIHLAQSTVWIAGATLLSAFNIERPVDQNGKPIDIPADFTTGFFRHPVP
FQCRFVPRTEQVSQSVSGP

Seq. ID No: 452

>PsiKchimera_1MKTKFCTGGEAEPSPLGLLLSCGSGLVPRGSPQPPA
DEQPEPRTRRRAYLWCKEFLPGAWRGLREDEFHISVIRGGLSNMLFQCSL
PDTTATLGDEPRKVLLRLYGAILQMRSCNKEGSEQAQKENEFQGAEAMVL
ESVMFAILAERSLGPKLYGIFPQGRLEQFIKMKTLLDYVTAKPPLATDIA
RLVGTEIGGFVARLHNIGRERRDDPEFKFFSGNIVGRTTSDQLYQTIIPN
AAKYGVDDPLLPTVVKDLVDDVMHSEETLVMADLWSGNILLQLEEGNPSK
LQKIYILDWELCKYGPASLDLGYFLGDCYLISRFQDEQVGTTMRQAYLQS
YARTSKHSINYAKVTAGIAAHIVMWTDFMQWGSEEERINFVKKGVAAFHD
ARGNNDNGEITSTLLKESSTA

Seq. ID No: 453

>PsiKchimera_2MAFDLKTEDGLITYLTKHLSLDVDTSGVKRLSGGFV
NVTWRIKLNAPYQGHTSIILKHAQPHMSTDEDFKIGVERSVYEYQAIKLM
MANQEVLGGGDSRVSVPEGFHYDVENNALIMQDVGTMKTLLDYATAKPPL
STEIASLVGTEIGAFIARLHNLGRKRRDQPAFKFFSGNIVGRTTADQLYQ
TIIPNAAKYGINDPLLPTVVKDLVEEVMNSEETLIMADLWSGNILLQLEE
GNPSELKKIWLVDWELCKYGPASLDMGYFLGDCYLIARFQDELVGTTMRK
AYLKSYARTASDTINYSKVTASIGAHLVMWTDFMKWGNDEEREE

Seq. ID No: 454

>PsiMchimera_1MDSAGNIYRHKVDFTALALQDPAFKETLSAKGRLDF
SNPDAVRQLTVSLLRRDFGLEVELPDDRLCPPVPNRLNYILWLQDLIDCT
GDDYHEGFNADRDVVGLDIGTGSSAIYPMLACARFKAWSMVGTEVERKCI
DTARLNVVANNLQDRLSILETSIDGPILVPIFEATEEYEYEFTMCNPPFY
DGAADMQTSDAAKGFGFGVGAPHSGTVIEMSTEGGESAFVAQMVRESLKL
RTRCRWYTSNLGKLKSLKEIVGLLKELEISNYAINEYVQGSTRRYAVAWS
FTDIQLPEELSRPSNPELSSLF

Seq. ID No: 455

>PsiMchimera_2MSATTNIYKEDIDFITLGREDSDFGKLLNSNGQLDF
SDPKSVQQLTKSLLKRDFGLKLILPDDRLCPPVPNRLNYVLWIEDIFNYT
NKTLGLSDDRPIKGVDIGTGASAIYPMLACARFKAWSMVGTEVERKCIDT
ARLNVVANNLQDRLSILETSIDGPILVPIFEATEEYEYEFTMCNPPFYDG
AADMQTSDAAKGFGFGVGAPHSGTVIEMSTEGGESAFVAQMVRESLKLRT
RCRWYTSNLGKLKSLKEIVGLLKELEISNYAINEYVQGSTRRYAVAWSFT
DIQLPEELSRPSNPELSSLF

Seq. ID No: 456

>PsiMchimera_3MAQNSTIYEDEVDFATLALQDSEFAKILKSNGQLDF
SNPESVQQLTKSLLKRDFKLKLSLPPDRLCPPVPNRLNYIIWIQNLLDTT
SDSYNDKYDPEREVLGLDIGTGASAIYPMLACARFKAWSMVGTEVERKCI
DTARLNVVANNLQDRLSILETSIDGPILVPIFEATEEYEYEFTMCNPPFY
DGAADMQTSDAAKGFGFGVGAPHSGTVIEMSTEGGESAFVAQMVRESLKL
RTRCRWYTSNLGKLKSLKEIVGLLKELEISNYAINEYVQGSTRRYAVAWS
FTDIQLPEELSRPSNPELSSLF

Seq. ID No: 457

>SAM2MSKSKTFLFTSESVGEGHPDKICDQVSDAILDACLEQDPFSKVAC
ETAAKTGMIMVFGEITTKARLDYQQIVRDTIKKIGYDDSAKGFDYKTCNV
LVAIEQQSPDIAQGLHYEKSLEDLGAGDQGIMFGYATDETPEGLPLTILL
AHKLNMAMADARRDGSLPWLRPDTKTQVTVEYEDDNGRWVPKRIDTVVIS
AQHADEISTADLRTQLQKDIVEKVIPKDMLDENTKYFIQPSGRFVIGGPQ
GDAGLTGRKIIVDAYGGASSVGGGAFSGKDYSKVDRSAAYAARWVAKSLV
AAGLCKRVQVQFSYAIGIAEPLSLHVDTYGTATKSDDEIIEIIKKNFDLR
PGVLVKELDLARPIYLPTASYGHFTNQEYSWEKPKKLEF

Seq. ID No: 458

>SAM3MDILKRGNESDKFTKIETESTTIPNDSDRSGSLIRRMKDSFKQSN
LHVIPEDLENSEQTEQEKIQWKLASQPYQKVLSQRHLTMIAIGGTLGTGL
FIGLGYSLASGPAALLIGFLLVGTSMFCVVQSAAELSCQFPVSGSYATHV
SRFIDESVGFTVATNYALAWLISFPSELIGCALTISYWNQTVNPAVWVAI
FYVFIMVLNLFGVRGFAETEFALSIIKVIAIFIFIIIGIVLIAGGGPNST
GYIGAKYWHDPGAFAKPVFKNLCNTFVSAAFSFGGSELVLLTSTESKNIS
AISRAAKGTFWRIAIFYITTVVIIGCLVPYNDPRLLSGSNSEDVSASPFV
IALSNTGSMGAKVSNFMNVVILVAVVSVCNSCVYASSRLIQALGASGQLP
SVCSYMDRKGRPLVGIGISGAFGLLGFLVASKKEDEVFTWLFALCSISSF
FTWFCICMSQIRFRMALKAQGRSNDEIAYKSILGVYGGILGCVLNALLIA
GEIYVSAAPVGSPSSAEAFFEYCLSIPIMIVVYFAHRFYRRDWKHFYIKR
SEIDLDTGCSVENLELFKAQKEAEEQLIASKPFYYKIYRFWC

Seq. ID No: 459

>SS02MSNANPYENNNPYAENYEMQEDLNNAPTGHSDGSDDFVAFMNKIN
SINANLSRYENIINQIDAQHKDLLTQVSEEQEMELRRSLDDYISQATDLQ
YQLKADIKDAQRDGLHDSNKQAQAENCRQKFLKLIQDYRIIDSNYKEESK
EQAKRQYTIIQPEATDEEVEAAINDVNGQQIFSQALLNANRRGEAKTALA
EVQARHQELLKLEKTMAELTQLFNDMEELVIEQQENVDVIDKNVEDAQQD
VEQGVGHTNKAVKSARKARKNKIRCLIICFIIFAIVVVVVVVPSVVETRK

Seq. ID No: 460

>T4H-CPR_1SSSSDVFVLGLGVVLAALYIFRDQLFAASKPKVAPVSTTK
PANGSANPRDFIAKMKQGKKRIVIFYGSQTGTAEEYAIRLAKEAKQKFGL
ASLVCDPEEYDFEKLDQLPEDSIAFFVVATYGEGEPTDNAVQLLQNLQDE
SFEFSSGERKLSGLKYVVFGLGNKTYEHYNLIGRTVDAQLAKMGAIRIGE
RGEGDDDKSMEEDYLEWKDGMWEAFATAMGVEEGQGGDSADFVVSELESH
PPEKVYQGEFSARALTKTKGIHDAKNPFAAPIAVARELFQSVVDRNCVHV
EFNIEGSGITYQHGDHVGLWPLNPDVEVERLLCVLGLAEKRDAVISIESL
DPALAKVPFPVPTTYGAVLRHYIDISAVAGRQILGTLSKFAPTPEAEAFL
RNLNTNKEEYHNVVANGCLKLGEILQIATGNDITVPPTTANTTKWPIPFD
IIVSAIPRLQPRYYSISSSPKIHPNTIHATVVVLKYENVPTEPIPRKWVY
GVGSNFLLNLKYAVNKEPVPYITQNGEQRVGVPEYLIAGPRGSYKTESFY
KAPIHVRRSTFRLPTNPKSPVIMIGPGTGVAPFRGFVQERVALARRSIEK
NGPDSLADWGRISLFYGCRRSDEDFLYKDEWPQYEAELKGKFKLHCAFSR
QNYKPDGSKIYVQDLIWEDREHIADAILNGKGYVYICGEAKSMSKQVEEV
LAKILGEAKGGSGPVEGVAEVKLLKERSRLMLDVWS

Seq. ID No: 461

>T4H-CPR_2SSSSDVLILGLGVALAALYLFRDQLFAASKPKAIPLTNKL
AGLDNEGNPRDFIAKMKAGKKRLVIFYGSQTGTAEEYAIRLAKEAKSKFG
LTSLVCDPEEYDFENLDQLPEECAVFFVMATYGEGEPTDNAVQLMQNLAD
ESFEFSGGERKLEGLKYVIFALGNKTYEHYNLIGRKVDTLLTDMGGVRCG
ELGEGDDDKSMEEDYLEWKDAMWEDFARKMGVEEGQGGDSADFAVSELDT
HVPEKVYLGELSARALTKTKGIHDAKNPYPAPIVASRELFQQGGDRNCVH
VELSIEGSGITYQHGDHVGVWPTNPEVEVNRLLCALGLWEKKDQVIGIES
LDPALAKVPFPVPTTYATVLRNYIDISAVTGRQILGHLSKYAPAPDVEEF
LKGLSTNKEQYGATVANGCLKLGEVLQLAAGNDLKAIPTTENTTAWSIPF
DVIVSAIPRLQPRYYSISSSPKLNPTSIHVTAVVLKYQSVASEKLPAKWV
YGVGSNFLLNLKYAANGEPAPFVTTNGSADPASVYYPTYAIEGPRGAYKQ
ETIYKSPIHVRRSTFRLPTNPKSPVIMIGPGTGVAPFRGFVQERVALARR
TIEKNGADALADWGRISLFYGCRKSTEDFLYKEEWPQYTEELKGKFNMHS
AFSREAPYKADGSKIYVQDLIWEDRANVSDAILNGKGYIYICGDAKSMAK
QVEDTLAKILGEAKGGTAEVEGAAEMKLLKERSRLMLDVWS

Seq. ID No: 462

>T4H-CPR_3SSSSSGAGADSDENPRDFIAKMKAGKKRLVIFYGSQTGTA
EEYAIRLAKEAKSKFGLTSLVCDPEEYDFENLDQLPEDCAVFFVMATYGE
GEPTDNAVQLMQNLQDESFEFSNGERKLEGLKYVVFALGNKTYEHYNLIG
RKVDTILGEMGAVRCGERGEGDDDKSMEEDYLEWKDAMWEDFARKMGVEE
GQGGDSADFAVSELESHAPEKVYLGELSARALTKTKGIHDAKNPYPAPIV
ESRELFQVGGDRNCVHVELGIEGSGITYQHGDHVGVWPTNPEVEVTRLLC
ALGLWEKKDQVIGIESLDPALAKVPFPVPTTYITVLRNYIDISAVTGRQI
LGHLSKFAPSPDAEAFLKSLSTNKEQYGAIVANGCLKLGEVLQLAAGNDL
KAVPNAENTTKWTIPFDVIVSAIPRLQPRYYSISSSPKLNPTTIHVTAVV
LKYESVASEKVPAKWVYGVGSNFLLNLKYAANGDAAPFVTANGSADPASV
YAPTYAIEGPRGAYKQETIYKSPIHVRRSTFRLPTNPKSPVIMIGPGTGV
APFRGFVQERVALARRTIEKNGPDALADWGRITLFYGCRKSTEDFLYKDE
WPQYTEELKGKFTMHSAFSREPPYKADGSKIYVQDLIWEDREKVADAILN
GKGYVYICGDAKSMAKQVEDTLAKILGESKGGSAEVEGAAEMKLLKERSR
LMLDVWS

Seq. ID No: 463

>T4H-CPR_4SSSSSSKLSDGDENPRDFIAKMKNGKKRLVIFYGSQTGTA
EEYAIRLAKEAKSKFGLTSLVCDPEEYDFENLDQLPDDCAAFFVVATYGE
GEPTDNAVQLMQNLQDESFEFSGGERKLEGLKYVVFALGNKTYEHYNVIG
RIVDTELAKMGAIRCGERGEGDDDKSMEEDYLEWKDGMWEEFARIMGVEE
GQGGDTPDFKVTELQSHPSEKVYLGELSARALTKTKGIHDAKNPYPAPIL
KSRELFQKQGERNCVHLELGIDGSGITYQHGDHVGVWPSNPEVEVNRLLC
ALGLWDKRDHVIGIESLDPALAKVPFPVPTTYSTVLRNYIDISAVAGRQI
LGNLARFSPSPDAEGFMRSLNTDKEQYGRIIANGCLKLGEVLQLAAGNDI
KAVPTLENTTAWPIPFDVIVSAIPRLQPRYFSISSSPKLHPTAIHVTAVV
LKYQSVASDKVPPKWVYGVGSNFILNLKYAACGETAPLIAQNGSADPAHT
PFPLYAIEGPRGAYKQEMIYKSPIHVRRSTFRLPTNPKSPVIMVGPGTGV
APFRGFVQERIALARRTIEKNGPDALADWGRISLFYGCRKSNEDFLYNEE
WPQYIDELKGKFTLHTAFSREPPYKPDGSKIYVQDLLWDDRSKVADAIIN
GKGYIYICGDAKSMAKSVEDVLAKILGEAKGGTMEVEGAAELKLLKERSR
LMLDVWS

Seq. ID No: 464

>T4H_1MKTRTSKHPPGPRGLPLIGNLLDMPASYEWLQYRKWSEEFKSDI
IYLNILGTQIVVTNTLESTLDLLEKRSSKYSGRHSFQLPNNCAMGWAWNL
ALMSYGDEWRAHRRLAARGFDAQAMPKFNHAFTRNTRGLLRRLLESPEAW
NEHVRHEVGSMIIEITYGLDVLSKNDPFIESADKGLATLALAVVPGAFLV
DTLPILKHIPSWFPGAGFKRKAKEWKRYADEVLEAPYKALKEEMASGAAK
PSFVQRCLQDMDPNIDTTNQERVIKNTAAEMYVAGADTSASFIATFVLAM
IQYPQVQRRAQAELDSVLGPDRLPTFGDMPSLPYLSAITKECFRWEVITP
ISIPHMLTEDDEYRGWFLPSGTVVIPNSWAIMNDPTVYPDPSVFNPERFL
KDGKIDLEVQDPQLAAFGYGRRICPGMRVANAFTWLSAGSILASFNISKP
AAKDGTPIELDVKYRSSSIRHPEAFDCLFKPRSENTRDMIVSAAA

Seq. ID No: 465

>T4H_2MSKRSKHPPGPRGLPLIGNLLDMPTNDEWLQYRKWSQEFKSDII
YLNVCGTQIVVTNTLESTLDLLERRSSKYSGRMGLEWAFILMPYGDEWRA
HRRLAAKGFDAKAIPKFNPTFTRNAQDLLRRLLESPEAWHEHVRHQVGAM
IIEVSYGLDVLHKNDPFIESADKAAVTFAMAIKPGAFLVNTVPILKYVPS
WFPGAGFQRKAKEWKRYNDAVLEAPFKALKEEITNGAARPSFAQQCLQNM
DPNIDTAYQERVIKDTSAAMYGGGSDTSVSFLATFVLAMLQYPSVQRRAQ
VELDSVLGRDRLPTFDDMPDLPYLAAVMKECHRWEIVLPLAIPHMLTADD
EYRGWFLLSGTLVIPNSWAILNDPTVYPDPSTFNPERFLKDGKIDPNVQD
PELAAFGYGRRTCPGRRITNAFTWLSAGYILASFNIENAVGNDGMPIEPK
VKYRSETIRHPDTFECVFTPRSDDTRDMIGSAYT

Seq. ID No: 466

>T4H_3MGRWPIIGNLLDMPQKSPWLTYAKWSEDCDSDIIHLNVLGTSIV
VLSSLEAISTLLEGKAVDFSDRPKSTMMSELMGWERGFAFMPYGQLWRSH
RKAFHQEFSPQVAHRNHPKLIKATHNLLRLLLNTPQHWHGHIRRQAGASI
MDIAYGIEVLPENDPYLDIAEAAVKAFNDASVPGAFLVDSIPLLKHVPAW
VPGAGFQLKAKEGRQALENLIDSPYNAMKKDLAGGKAKSSYTSRSLAAMD
ATGVIEENETIIRETAAMVYLGGSDSTPSTTSVFILAMLAHPEVQRKAHA
ELDSVIGKAQLPTFKDRGSLPYVTAVAKEVLRWEPVAPLAVPRKVRVDSE
YKGYRIPKGSIVFQNSWALLHDEKTYPNPLAFNPERFLKDGQLDPNVQDP
DVVAFGYGRRSCPGKTMGYDSVWLNVASILAAFDIKKVANPDSTNVEPKF
EPFGITV

Seq. ID No: 467

>T4H_4MYLFKAYLRPSRRLPPGPRGWPLIGNLLDMPTSDEWVRYAQWVR
EFKSDVIHLEVCGTHIVILNSVESAVDLLEKRSSLYSSRPPTPMMSDLMG
WSWNTAMLPYNDEWRAQRRHFHGEFDGRAIGKHYPPIIRSTHDLLQRLLD
TPEQWQSHIRHLVGATILDVAYGIEVLPADDPYVRTAEAAFASVSEAMVP
GAFLVDVLPILKHMPSWMPGAGFKRKAVAWKKLADAVFDAPFAAMKQAMA
AGTAKSSFGSRSLRDIDIKGNVQSQEFSIQAAAGTMYNAGSDTTVALLET
FMLAMVLHPEVQTKAQAEMDLVLGRSNLPTFADQESLSYLAAVMQEVFRW
QVVAPFGVPHMSTADDEYRGYFIPEGTIVIPNAHQMLNDEDVYPEPSKFK
PERFLKDGKLDLSVRSPLIAAFGFGRRICPGRALGENSAWLAAGSILTMF
NLSKATDHNGVTIEPSGRYTSGLVRHPETFKCQITPRSNEPRRELAGEIE
LITGRIQESEEA

Seq. ID No: 468

>T4H_CPR_chimera_1SSSSSGSVAYFTKGTYWAVPKDPYASSYGAAN
GAKAGKTRDIIEKMEETGKNCVIFYGSQTGTAEDYASRLAKEGSQRFGLK
TMVADLEEYDYENLDKWPEDKVAFFVLATYGEGEPTDNAVQLLQNLQDES
FEFSSGERKLSGLKYVVFGLGNKTYEHYNLIGRTVDAQLAKMGAIRIGER
GEGDDDKSMEEDYLEWKDGMWEAFATAMGVEEGQGGDSADFVVSELESHP
PEKVYQGEFSARALTKTKGIHDAKNPFAAPIAVARELFQSVVDRNCVHVE
FNIEGSGITYQHGDHVGLWPLNPDVEVERLLCVLGLAEKRDAVISIESLD
PALAKVPFPVPTTYGAVLRHYIDISAVAGRQILGTLSKFAPTPEAEAFLR
NLNTNKEEYHNVVANGCLKLGEILQIATGNDITVPPTTANTTKWPIPFDI
IVSAIPRLQPRYYSISSSPKIHPNTIHATVVVLKYENVPTEPIPRKWVYG
VGSNFLLNLKYAVNKEPVPYITQNGEQRVGVPEYLIAGPRGSYKTESFYK
APIHVRRSTFRLPTNPKSPVIMIGPGTGVAPFRGFVQERVALARRSIEKN
GPDSLADWGRISLFYGCRRSDEDFLYKDEWPQYEAELKGKFKLHCAFSRQ
NYKPDGSKIYVQDLIWEDREHIADAILNGKGYVYICGEAKSMSKQVEEVL
AKILGEAKGGSGPVEGVAEVKLLKERSRLMLDVWS

Seq. ID No: 469

>T4H_CPR_chimera_2SSSSSSGTIAYFTKGTYWGIVKDPYAPNYPPA
NGNKPAKTRNIVEKMDESNKNCVVFYGSQTGTAEDYASRLAKEGKSRFGL
ETMVADLEDYDFDNLDTLGDDKVAIFVLATYGEGEPTDNAVQLLQNLQDE
SFEFSSGERKLSGLKYVVFGLGNKTYEHYNLIGRTVDAQLAKMGAIRIGE
RGEGDDDKSMEEDYLEWKDGMWEAFATAMGVEEGQGGDSADFVVSELESH
PPEKVYQGEFSARALTKTKGIHDAKNPFAAPIAVARELFQSVVDRNCVHV
EFNIEGSGITYQHGDHVGLWPLNPDVEVERLLCVLGLAEKRDAVISIESL
DPALAKVPFPVPTTYGAVLRHYIDISAVAGRQILGTLSKFAPTPEAEAFL
RNLNTNKEEYHNVVANGCLKLGEILQIATGNDITVPPTTANTTKWPIPFD
IIVSAIPRLQPRYYSISSSPKIHPNTIHATVVVLKYENVPTEPIPRKWVY
GVGSNFLLNLKYAVNKEPVPYITQNGEQRVGVPEYLIAGPRGSYKTESFY
KAPIHVRRSTFRLPTNPKSPVIMIGPGTGVAPFRGFVQERVALARRSIEK
NGPDSLADWGRISLFYGCRRSDEDFLYKDEWPQYEAELKGKFKLHCAFSR
QNYKPDGSKIYVQDLIWEDREHIADAILNGKGYVYICGEAKSMSKQVEEV
LAKILGEAKGGSGPVEGVAEVKLLKERSRLMLDVWS

Seq. ID No: 470

>T5H-CPR_1SSSSSSGGLLAFLYLFRGTLFASGKASDAGSKLAGGSDLD
SSADAAANDFVTKLTSQNKRIAIFYGSQTGTAEEYATKIAKEAKARFGTS
SLVCDPEEYEFEKLDQLPSDCVACFVMATYGEGEPTDNAVGLMEFLDGED
VQFSNGSSLDNLNYVIFGLGNRTYEHYNAIARKLDARLESLGAKRIGERG
EGDDDKSMEEDYLAWKDGMFEALASSLGFEEGGGGDVADFKVREVADHPE
DKVYRGELSARALLGTKGIHDAKNPYNAVVKEARELFVEGTADRTCVHVE
FDIEGSGISYQHGDHIAVWAHNPEQEVERALAVLGLLGKRDTVIDVESLD
PTLAKVPFPVPTTYEAVFRHYLDICAHASRQTLNNFAKYAPTPEARAKLE
KACGDKAAFQEAIGHRCLKTFEALQLIVGDDLGGDSVAKATAWEIPFDRV
ISDLPRVGPRFYSISSSPKMHPKTVHITAVVLRYRPEAAGQDSPYVHGLA
TNFISAIKMAKNNEQPSGPDDPRFGTPGYDLAGPRGAYTKESLFRAPIHI
RRSNFRLPTSPKIPVIMVGPGTGVAPFRSFVQERVCSAQKTCDKVNQSPA
EALQDWGNIWLFYGCRRSNEDFLYKDEWPEYASKLGGKFQMETAVSREKF
KPDGSKLYVQDLIWERRKELAQDILDKKAYIYICGEAKGMAHDVEEMFGR
VLEEAKGSAEAGRRELKLLKERSRLLLDVWS

Seq. ID No: 471

>T5H-CPR_2SSSSSSLFSTTDVILFSLIVGVMTYWFLFRKKKEEVPEFT
KIQTTTSSVKDRSFVEKMKKTGRNIIVFYGSQTGTAEEFANRLSKDAHRY
GMRGMAADPEEYDLADLSSLPEIEKALAIFCMATYGEGDPTDNAQDFYDW
LQETDVDLSGVKYAVFALGNKTYEHFNAMGKYVDKRLEQLGAQRIFDLGL
GDDDGNLEEDFITWREQFWPAVCEHFGVEATGEESSIRQYELMVHTDMDM
AKVYTGEMGRLKSYENQKPPFDAKNPFLAVVTTNRKLNQGTERHLMHLEL
DISDSKIRYESGDHVAVYPANDSALVNQLGEILGADLDIIMSLNNLDEES
NKKHPFPCPTSYRTALTYYLDITNPPRTNVLYELAQYASEPTEHEQLRKM
ASSSGEGKELYLRWVLEARRHILAILQDYPSLRPPIDHLCELLPRLQARY
YSIASSSKVHPNSVHICAVAVEYETKTGRINKGVATSWLRAKEPAGENGG
RALVPMYVRKSQFRLPFKATTPVIMVGPGTGVAPFIGFIQERAWLRQQGK
EVGETLLYYGCRRSDEDYLYREELAGFHKDGALTQLNVAFSREQPQKVYV
QHLLKKDKEHLWKLIHEGGAHIYVCGDARNMARDVQNTFYDIVAEQGAME
HAQAVDYVKKLMTKGRYSLDVWS

Seq. ID No: 472

>T5H-CPR_3SSSSSSEAVAEEVSLFSMTDMILFSLIVGLLTYWFLFRKK
KEEVPEFTKIQTLTSSVRESSFVEKMKKTGRNIIVFYGSQTGTAEEFANR
LSKDAHRYGMRGMSADPEEYDLADLSSLPEIDNALVVFCMATYGEGDPTD
NAQDFYDWLQETDVDLSGVKFAVFGLGNKTYEHFNAMGKYVDKRLEQLGA
QRIFELGLGDDDGNLEEDFITWREQFWPAVCEHFGVEATGEESSIRQYEL
VVHTDIDAAKVYMGEMGRLKSYENQKPPFDAKNPFLAAVTTNRKLNQGTE
RHLMHLELDISDSKIRYESGDHVAVYPANDSALVNQLGKILGADLDVVMS
LNNLDEESNKKHPFPCPTSYRTALTYYLDITNPPRTNVLYELAQYASEPS
EQELLRKMASSSGEGKELYLSWVVEARRHILAILQDCPSLRPPIDHLCEL
LPRLQARYYSIASSSKVHPNSVHICAVVVEYETKAGRINKGVATNWLRAK
EPAGENGGRALVPMFVRKSQFRLPFKATTPVIMVGPGTGVAPFIGFIQER
AWLRQQGKEVGETLLYYGCRRSDEDYLYREELAQFHRDGALTQLNVAFSR
EQSHKVYVQHLLKQDREHLWKLIEGGAHIYVCGDARNMARDVQNTFYDIV
AELGAMEHAQAVDYIKKLMTKGRYSLDVWS

Seq. ID No: 473

>T5H-CPR_4SSSSSSAAAADGDGGQSRRLLALLATSLAVLVGCGVALLF
RRSSSGAAPLARQAAAAKPLAAKKDQEPDPDDGRQRVALFFGTQTGTAEG
FAKALAEEAKARYDKAVFKVLDLDDYAAEDEEYEEKLKKENIAFFFLATY
GDGEPTDNAARFYKWFSEGNERGEWLSNLQYGVFALGNRQYEHFNKVGKE
VDQLLAEQGGKRIVPVGLGDDDQCIEDDFNAWKELLWPELDKLLRVEDNS
SAAQSPYTAAIPQYRIVLTKPEDATHINKSFSLSNGHVVYDSQHPCRANV
AVRRELHTPASDRSCIHLEFDIAGTSLTYETGDHVGVYAENSTETVEEAE
KLLDYSPDTYFSIYADQEDGTPLFGGSLPPPFPPCTVRVALARYADLLNS
PKKSVLLALAAHASDPKEAERLRHLASPAGKKEYSQWIIASQRSLLEVIS
EFPSAKPPLGVFFAAIAPRLQPRYYSISSSPRMAPTRIHVTCSLVHGQSP
TGRIHKGVCSTWMKNSTPSEEESEECSWAPIFVRQSNFKLPADPTVPIIM
VGPGTGLAPFRGFLQERLALKETGVELGRAILFFGCRNRQMDFIYEDELN
NFTESGALSELVVAFSREGPTKEYVQHKMAEKAADLWSIVSQGGYVYVCG
DAKGMARDVHRALHTIVQEQVTQRTSNFGLWKFRLVSLN

Seq. ID No: 474

>T5H-CPR_5SSSSSSAAAAGGDPLAALAATAAALVAGVVILAVWFRSGG
GAPPKAAAPPPRPPPVKIEADADADDGRKRVTVFFGTQTGTAEGFAKAMA
EEARARYEKAVFKVVDLDDYAAEDEEYEEKLRKETIVLLFLATYGDGEPT
DNAARFYKWFTEGKEKEVWLKDLKYAVFGLGNRQYEHFNKVAKVVDELLE
EQGGKRLVPVGLGDDDQCIEDDFTAWKEQVWPELDQLLRDEDDTTGASTP
YTAAIPEYRIVFIDKSDVSFQDKSWSLANGSGVIDIHHPVRSNVAVRKEL
HKPASDRSCIHLEFDISGTGLVYETGDHVGVYSENAIETVEQAEKLLDLS
PDTFFSVHADAEDGSPRKGGGSLAPPFPSPCTLRTALLRYADLLNSPKKA
ALVALAAHASDLAEAERLRFLASPAGKDEYSQWVVASQRSLLEVMAAFPS
AKPPLGVFFAAVAPRLQPRYYSISSSPKMAPSRIHVTCALVYGPTPTGRI
HQGVCSTWMKNAIPSEYSEECSWAPIYVRQSNFKLPADPTTPIIMIGPGT
GLAPFRGFLQERLALKQSGVELGNSVLFFGCRNRNMDYIYEDELQNFIQE
GALSELIVAFSREGPAKEYVQHKMTEKATEIWNIVSQGGYIYVCGDAKGM
ARDVHRALHTIVQEQGSLDSSKTESYVKSLQMDGRYLRDVW

Seq. ID No: 475

>T5H-CPR_6SSSSSSAAAYLFRDQIFRSSSPKVVVPAPSKLANGHGNPR
NFVSKMKEGKKRIVIFYGSQTGTAEEYAIRIAKEAKTKFGLTSLVCDPEE
YDFENLDQVPEDCCVFFVMATYGEGEPTDNAVQLMQNLEDESFEFSNGSH
RLDGLKYVVFALGNKTYEHYNAIGRKVDTLLTDMGATKIGERGEGDDDKS
MEEDYLEWKDGMWKAFSEAMGVEEGQGGDTPDFAVTELDSHPPEKVYLGE
LSARALTRTKGIYDGKNPYPSAVKHSRELFQAGAERNCVHAELDIEGSGI
TYQHGDHVGVWPSNPDVEVDRMLYVLGLYGKKDAVINIDSLDPALAKVPF
PVPTTYATVLRHYIDICAVAGRQMLGVLSKFAPHPKAEAFLKSLNSDKEE
YSNIVTNGCFKLGEVLQLAAGDDIKLCPTPDNTTAWAIPFDIIVSSIPRL
QPRFYSISSSPKLYPNAIHLTAVVLKYDSIPNRLVESRFVYGVATNFLLN
VKYAANGETAPFIAEPVISEPAHVSLPKYAIEGPRGAHIEDNIYKIPIHV
RRSTFRLPANPKIPVIMVGPGTGVAPFRGFVQERVALAKRSIEKNGPDAL
ADWGSITLFYGCRKSNEDFLYKEEWPQYAEELKGKFKMHCAFSREPPYKP
DGSKIYVQDLIWEERETIAKAILEGKAYVYICGDAKAMSRAVEDTLARIL
GEAKGGNAEVEGAAEMKILKERSRLLLDVWS

Seq. ID No: 476

>T5H-CPR_7SSSSSSSLFSTTDMVLFSLIVGVLTYWFIFRKKKEEIPEF
SKIQTTAPPVKESSFVEKMKKTGRNIIVFYGSQTGTAEEFANRLSKDAHR
YGMRGMSADPEEYDLADLSSLPEIDKSLVVFCMATYGEGDPTDNAQDFYD
WLQETDVDLTGVKFAVFGLGNKTYEHFNAMGKYVDQRLEQLGAQRIFELG
LGDDDGNLEEDFITWREQFWPAVCEFFGVEATGEESSIRQYELVVHEDMD
VAKVYTGEMGRLKSYENQKPPFDAKNPFLAAVTANRKLNQGTERHLMHLE
LDISDSKIRYESGDHVAVYPANDSALVNQIGEILGADLDVIMSLNNLDEE
SNKKHPFPCPTTYRTALTYYLDITNPPRTNVLYELAQYASEPSEQEHLHK
MASSSGEGKELYLSWVVEARRHILAILQDYPSLRPPIDHLCELLPRLQAR
YYSIASSSKVHPNSVHICAVAVEYEAKSGRVNKGVATSWLRAKEPAGENG
GRALVPMFVRKSQFRLPFKSTTPVIMVGPGTGIAPFMGFIQERAWLREQG
KEVGETLLYYGCRRSDEDYLYREELARFHKDGALTQLNVAFSREQAHKVY
VQHLLKRDREHLWKLIHEGGAHIYVCGDARNMAKDVQNTFYDIVAEFGPM
EHTQAVDYVKKLMTKGRYSLDVWS

Seq. ID No: 477

>T5H-CPR_8SSSSSSGGSPMSDSVVVIITTSFAVIIGLLVFLWKRSSDR
SKEVTPLVVPKSLSVKDEEDEAETLAGKTKVTIFYGTQTGTAEGFAKALA
EEIKARYEKAAVKVVDLDDYAMDDDQYEEKLKKETLTFFMVATYGDGEPT
DNAARFYKWFTEEHERGVWLQQLTYGIFGLGNRQYEHFNKIAKVLDEQLN
EQGAKRLIPVGLGDDDQCIEDDFTAWRELLWPELDNLLRDEDDVNGASTP
YTAAIPEYRVVIHDASATSCEDKSVLENGNTSIDIHHPCRVNVAVQKELH
KPESDRSCIHLEFDISGTGIIYETGDHVGVYAENFEENVEEAGKLLGQPL
DLLFSIHADNEDGAPLGSSLAPPFPGPCTLRTALSHYADLLNPPRKAALI
ALAAHASEPSEAERLKYLSSPEGKDEYSQWIVGSQRSLLEVMAEFPSARP
PLGVFFAAIAPRLQPRYYSISSSPRFALSRVHVTCALVYGPTPTGRIHKG
VCSTWMKNAVPLEKSHDSSWAPVFIRTSNFKLPTDPSIPIIMVGPGTGLA
PFRGFLQERMALKEDGAQLGPALLFFGCRNRRMDFIYEDELNYFVEQGVI
SELIVAFSREGPQKEYVQHKMMDKAAQIWSLISERGYIYVCGDAKGMARD
VHRTLHTIVQEQGNLDSSKTESMVKKLQMDGRYLRDVW

Seq. ID No: 478

>T5H-CPR_9SSSSSSTSFHKLKRILHKHLQRSHSIGAECKPQRSNHEDL
LAVMNRSSIKVSIFYGSQTGTAKKFAINLGHHLHNCGVRNLVMDLRQTNM
EILVNLSMLDNCVALFVVATYGEGEPTDSARQFMDNLKNSYQKLDNLRFA
VFGLGNSMYTYFNAVGKSIDRLLIQHGGKRLQTLTLGDEVNELESTFLNW
RSHLTSLLIDFFDLNDHDRNYLNKQYKRMYSLKRFNWNVPLVSHFVNMFI
NKAHVKETLPYENDNYFYASVAVNQELYHKSSRSCRHIELDVSASQLRYK
TGDHIAIFASNPLDLVEKIGDLLNIDLNEMISLDAVDPDSLTKHPFPCPC
TYRHAFMHFVDITGPPGKSLLSACLDSVTNPEESQFVQLLISDSEDGKKL
YSKWILEDHRGLVDVLQDLKSFRPPADLLLELLNPLKPRLYSISSSSLVH
TNRIHITASIVKYKTNSGRIFKGLATNWLKSLQSTNTERHLKIPVAIHTS
NFNLPRSRTIPVIMIASGTGLAPFRAFIQERLKVAHDKVGKTGQMVLFFG
CRHENKDFIYSDELKQACSTGLLEMFTAFSRDCLDGNKVYVQHKVLEMGN
MVWKLLDECYAYIYVCGDAAGMVRDVHLCLIELVVQRSNLTREAATSYVL
NLRKQGRYRTDVWK

Seq. ID No: 479

>T5H-CPR_10SSSSSSGGKIFDKLNSSLDSGDSTSPASLTALLMENKDL
MMILTTSVAVLIGCAVVLMWRRSSTSARKVVELPKLVVPKSVVEPEEIDD
GKKKIAIFFGTQTGTAEGFAKALAEEAKARYEKAIFKVIDMDDYAADDEE
YEEKLKKEKLAFFFLATYGDGEPTDNAARFYKWFEEGKERGDCFKNLQYG
VFGLGNRQYEHFNKIAKVVDELLAEQGGQRLVPVGLGDDDQCIEDDFAAW
RELVWPELDKLLLDGDDATATTPYTAAVLEYRVVTYDKSNFDNDLTNTNG
HANGHVIVDAQHPVRANVAVRKELHTPASDRSCTHLEFDISCTGLTYETG
DHVGVYCENFVETVEEAERLLNISPDTFFSIHTDKEDGTPLGGSSLPSPF
PPCTLRTALTRYADVLSSPKKSSLLALAACSSDPNEADRLRYLASPAGKE
EYAQWIVASQRSLLEVMAEFPSAKPSIGVFFASVAPRLQPRFYSISSSPR
MAASRIHVTCALVYDKMPTGRIHKGVCSTWMKNAIPLEESLSCSTAPIFV
RQSNFKLPADNKVPIIMIGPGTGLAPFRGFLQERMALKEEGADLGPAVLF
FGCRNRQMDYIYQDELDNFLEAGALSNLVVAFSREGPNKEYVQHKMTQKA
DDIWNMISQGGYVYVCGDAKGMARDVHRTLHTIAQDQGSLDSSKAESFVK
NLQTTGRYLRDVW

Seq. ID No: 480

>T5H-CPR_11SSSSSSGGDGAEGRALVATLAAAVLGAALFVLWRRAAAG
KKRKREAAAAAVAEATEVKARAAKGGEDEKAADDGRKKVTVFFGTQTGTA
EGFAKALAEEAKARYDKAIFKVVDLDDYAAEDEEYEEKLKKEKLALFFVA
TYGDGEPTDNAARFYKWFTEGNERGVWLNDFEYAVFGLGNRQYEHFNKVA
KVVDEILTEQGGKRLVPVGLGDDDQCIEDDFNAWKEALWPELDRLLRDEN
DASTGTTYTAAIPEYRVEFIKPEEAAHLERNFSLANGHAVHDAQHPCQAN
VAVRRELHTPASDRSCTHLEFDIAGTGLTYETGDHVGVYTENCPEVVEEA
ERLLGYSPDTFFTIHADKEDGTPLSGSSLAPPFPSPITVRNALARYADLL
NSPKKTSLVALATYASDPAEADRLRFLASAAGKDEYAQWVVASQRSLLEV
MAEFPSAKPPLGVFFAAVAPRLQPRYYSISSSPSMAATRIHVTCALVHET
TPAGRVHKGVCSTWIKNAVPSEESKDCSWAPIFVRQSNFKLPADPSVPII
MIGPGTGLAPFRGFLQERLAQKESGAELGPSVFFFGCRNSKMDFIYEDEL
NNFLEQGALSELVLAFSRQGPTKEYVQHKMAQKASEIWDMISQGAYIYVC
GDAKGMARDVHRVLHTIVQEQGSLDSSKAESFVKNLQMEGRYLRDVW

Seq. ID No: 481

>T5H-CPR_12SSSSSSVRESSFIEKMKKTGKNIVVFYGSQTGTGEEFAN
RLAKDAHRYGMRGMAADPEEFEMTDLSRLTEIENALAVFCMATYGEGDPT
DNAQDFYDWLQETDIDLAGLKYAVFGLGNKTYEHFNAMGKYVDKRLEELG
AERIFELGMGDDDGNLEEDFITWREQFWPAVCEHFGVEATGEDSSIRQYE
LVVHTDENMNKVYTGEMGRLKSYETQKPPFDAKNPFLANATVNRKLNEGG
DRHFMHLELDITGSKIRYESGDHVAVYPANDAALVNKLGEILGADLETVI
SLNNLDEESNKKHPFPCPTTYRTALTYYLDITNPPRTNVLYELAQYATDS
KEQENLRKMASSAQDGKALYLSWVVESRRNILAILEDIPSLRPPLDHLCE
LLPRLQARYYSIASSSKVHPNSIHVCAVLVEYETKTGRENKGVATNWLKN
KQPSDNGHKSSVPMFVRKSQFRLPFKPSTPVIMIGPGTGIAPFMGFIQER
EWLKQQGKDVGETVLYYGCRHEHEDFLYINELKRYHKEGVLTQLNVAFSR
DQAHKVYVQHLLKNNKEMVWKLIHEDNAHIYVCGDARNMARDVQNIFYDI
VEEYGKLDHAQAVDYIKKLMTKGRYSQDVWS

Seq. ID No: 482

>T5H_1MLPIVDHLLDVLNLERTPFRTYAVTALLLLFVGIIARALLKMML
FIQEYSANSKRLRCFPEPPNRSWILGHLGLFAPNEEGMTEFSKQVSKFTY
YMKTWMGPVIPLISLIHPDTIKPVVAAPASIAPKDALFYGFLEPWLGDGL
LLSRGEKWVRHRRLLTPAFHFDILKHYVKIFNQSTDIMHAKWRRLCTKGP
VFLDMFEHISLMTLDSLLKCTFSYDSDCQEKPSDYIAAIYDLSELIVERE
QCPPHHFDFIYRFSSNGRKFQRACRIVHEFTANVVQQRKKALQEKGAENW
IRSKKGKTQDFIDILLLSKDEDGNTLSDQEMRDEVDTFMFEGHDTTASGL
SWILYNLASHPEYQEKCREEVTQLLKGESTHLEWDDLSLLPFTTMCIKES
LRLHPPVTAVSRRCTEDIAMPDGKVIPKGNISLISIYGTHHNPAVWPNPE
VYDPYRFDPSSTDERSSHAFVPFSAGPRNCIGQNFAMAEMKVVLALTLLN
FKVALDPNRVVRRKPELVLRAEGGLWLQVEALKSKS

Seq. ID No: 483

>T5H_2MELLGLVSWLLLLLLTLVVICFLLYCGYIHYQHMKYDHIPGPPR
ESFLFGHGSAIWKVMRKNQLVYDLFLNWVETYGPVIRINALHKVTIVSVS
PESVKEVLMSPKYRKDWFYDHLHSLFGVRLMGNGLVTDRDNDHWYKQRRI
MDPAFSRTYLIGLLGPFNEKAEELMERLAEEADGRSHVVMHAMMSRVTLD
VISKVAFGMEMNSLKDDGTPLPRAISLVMRALVEMRNPFIRYSREKQAFI
RDVQESARLLRKTGRECIERRQKAIQDGEEIPVDILTQILKGAALEGDCD
MEDLLDNFVTFFIAGQETTANQLAFTIMELARNPEILEKAQAEVDEVIGV
KRDIEYDDLGKLQYLSQVLKESLRLYPTAPGTSRAIEEETIIEGFRIPPK
VPLMFNSYIMGRMQQFYPDPLTFNPDRFHPDAPKPYYSYFPFSLGPRSCI
GQVFAQMEAKVIMAKLLQRFQFELVEGQSFGIMDTASLRPEGGVICRLTI
RTNPGKAKKDD

Seq. ID No: 484

>T5H_3MSRPQVPKGLKNPPGPWGWPLIGHMLTLGKNPHLALSRMSQQYG
DVLQIRIGSTPVVVLSGLDTIRQALVRQGDDFKGRPDLYTFTLISNGQSM
SFSPDSGPVWAARRRLAQNGLKSFSIASDPASSTSCYLEEHVSKEAEVLI
STLQELMAGPGHFNPYRYVVVSVTNVICAICFGRRYDHNHQELLSLVNLN
NNFGEVVGSGNPADFIPILRYLPNPSLNAFKDLNEKFYSFMQKMVKEHYK
TFEKGHIRDITDSLIEHCQEKQLDENANVQLSDEKIINIVLDLFGAGFDT
VTTAISWSLMYLVMNPRVQRKIQEELDTVIGRSRRPRLSDRSHLPYMEAF
ILETFRHSSLVPFTIPHSTTRDTSLKGFYIPKGRCVFVNQWQINHDQKLW
VNPSEFLPERFLTPDGAIDKVLSEKVIIFGMGKRKCIGETVARWEVFLFL
AILLQRVEFSVPLGVKVDMTPIYGLTMKHACCEHFQMQLRS

Seq. ID No: 485

>T5H_4MPTPGGRLVAFLQRRGKLAGSLAVILLLILKRLRDAPRKVRWLR
GPPLLGVVLKVFQGLREHALLDMYDRWHQRLGPTFAYCAPGKMVVATIDP
KNIEHVLKTKFDNYVKGHVFAEPFTDLLGDGIFNADGEMWHRQRKTASRM
FTKRQFETHIWKAIEANTAKVGRILERSEGTLDMFNLMNRFTLDTIGRIG
FSKDIGSLEDPSSPFLRSFDRAQQILILRFWTNPAWKVLRWLGVGWEREL
KEHLGRLDGYARGIVRELRQKAEAGQDDSFVGLFMKEEQAAPAARSPELQ
EKFMRDLVLNFLIAGRDTTAQCISWTLFELTQHPAVAAKARQEVLDVCGE
GPVTFEHLKSLQYVRAILDEGLRLHPSVPYDGKLCLGKDTLPDGTVVPAG
CIIQYIPYAQGRCKDIWGEDACSFRPERWLEMPRRPSSFAFAAFNAGPRE
CLGRRLAEAEMAALVSTVVRDFDMRLEVEPSSVRYDAQLTLGMCGLPVSV
RRCRRAYGVAEPLAGA

Seq. ID No: 486

>T5H_5MLPLRHKMLTGEAEPCLVSKTAETDAEWTRDAFGMGQYTAGRCD
HLLSWVVFLLLAPVLLIVWLPLSCICCASPVLLVQRFAGWVLSGCLARTY
LGVLLIRLCGKCDLILTGMHFIRTGSQRFWMDTLDPQDWAYHNETYGRNI
ILWANLRVGSYKQVRDIVLNPARKRTRALDGWISGFARHYPNLPVFFNTG
SNMHTTFRQIFFANFTKTDFVLRALEDEGAGLAKMAAPILQRWLAGSFRE
SKSGEGNLYMVEPVAPLILFLLFEVEVESIPPELLTAFSDVVTVGASYFL
LPPHSPYWLLSGKVKAIALLKDFLLEHCNAARPESLKGRAVDWRSLAAQM
PAFLPKDECRCPCSGTPAVDPVDAYLEVISVMVCVAGVTGTTNGFTSVIR
KFADVPVGPTKSRWPSAPVQWRPDADDMVRLYRRDPLGFILEALRLGTPV
AGTHQVLEEELTCPFLHKETTFPKGTVVCANLNACHTDPEEWGSDALEFR
PGRAARNRYLMWNGPFGEAAPRQCPGEQVAAHCIKVSIDAFLDMHKPQ

Seq. ID No: 487

>T5H_6MATSILSLSLMDLLYWGACLCVLSVLYKISALYLRQKNFERVFS
AFPGPKRHWLYGNAHEFKQDGTDLDILNGYAKQFDCAFPLWLGNFFASLA
IYHPDYIKAILSRQDPKDNFVYHFITPWIGKGLLVLSGQKWYQHRKLLTP
GFHYDVLKPYVGVMSDCVNVMLDKWERLVPDKKPVELFHYISLMTLDTIM
KCAFSYQSNCQNDSENEYIKAVYELSYLVDHRTRCPPYHNDFIFYWSPHG
FRLRRALKTAHQHTEKVIKLRKESLKQETELEKIKQKRRLDFLDILLCAR
DENGQGLSDEDLRAEVDTFMFEGHDTTASGVSWTFYCLAKNPEHQEKCRE
EIRQVLGDRRTVEWEDLSKLPYTTMCIKESMRLYPPVPEVARELKEPITF
CDGRSVPKGSIVFLCIYAINRCPGIWEDPEVFDPLRFSPENSSTRHSHAF
LPFSAGGRNCIGQNFAMNEMKIATALTLQRFELQLETKREPVKRAQLVLR
SMNGIYINLKKIHSDKTKII

Seq. ID No: 488

>T5H_7MGLWTFMTGALILLLILVVLCFLLYCGYIYYMHMKYDHIPGPPR
DSFFFGHSPTIMKLMRNNVIMYDTFLEWVKTYGPVVRVNLSCSTIVFVIS
QEAVKEFLMSPKYTKDNFYECVETLFGVRYMGKGLLTDRDYEHWHKQRRI
MDPAFSRNYLIGLMGTFNEIAEDLVDILGDKADGKCQVGMHDMMGRVTLD
IIAKVAFGMELNSLHDDQTPFTRAITTVMRGMVETRNPLARYIPGKQALI
RDIKESLKFLRKTGRECILQRRKAIQDGEDIPRDILTQILKGAETEGDCS
LENLIDNFVTFFIAGQETTANQLSFAVMELGRHPEILTRVQAEVDEVLGS
KRDIEYEDLGKLQYLSQVLKETLRLYPIAPGTSRALEKEMVIEGVRVPPG
TTLMFNAYIMGRMEKYYHDPLVFNPDRFHPDAPKISYAYLPFSLGPRSCI
GQVFAQMEAKVVMAKLLQRFEFELVEGQSFRILDTGTLRPLDGVICRLRP
RAEHKSRK

Seq. ID No: 489

>T5H_8MWTILLSTINITLATALMLSFIIIYLLYIQNSTKLPPGPTSWPL
IGYTSCLGTDAFRKIQDLNKIYGDIVSFQVLGKTIIILYNYDLIHEAANG
NRSKVGRYTMTVNDLLAENSGISNYDTQKALEMRKAFVRLVHNNIKTTEE
HEGNKLQPFISQNIINAQINKLIRQLRIRQGKPVNVLQLMRCTVWRIIWN
LIFGKECQLTDKQISDTLDDISSNNLQNQLFQIRQLLPRFCVNIFKHSQF
ARKLFEIEEIIYKYKTVRQLIDNNVGEMHNSDSLLGQLINDLKLNLTKND
ISRLSFEFMAAGTDTTSLTLTWACDYLARAPPKESLKLSSDLIDMIHRWA
SVVPLSLPHIVRESFKLKNYYIPKSSILIYNLYAVHNSQIKKLINTEQNS
DEIQESDKPIPFSLGSRSCPGARIANLLIEQILTAINQEFLIQNITQSPF
ETISPGNQESLTPFGITRTPHKSMYIFVTKLNGNRRTSI

Seq. ID No: 490

>T5H_9MSQLLSSLIELPTQTLVLATAVAVGAAALLVHAYLFDAVGKHGN
LPPGPPVDSLFSGHRIPSTHPWRYLEKLTEEYGDIFTLRIGRSPLFVLGR
ASSAHRILEKQSALSSSRPRLVLAGELLSNNKRILLMPYGDQWRLYRKAM
HETLNDTVAKQYEPIQEREARIATLHLGRLGQADGGGGDFQRVLHRYAAS
VIMQVTYDYQVQTLDDPLVRSVAQRGHALAMCIRPGASVLDRYPLLEHVP
TWLNPWKQEGLRLRKLEQELYLGQVIKVRERMERGECAPCFVSKMTERQQ
ELGLTDLDVAGMSGSLFGAGSDTTASALSIFVMAVCRYPAVLARLHEELD
RVVSRDRMPTFDDIPQMPYVRATVQEVLRWRPVSAGGFQHSLTADVEYKG
YVLPKGSTVVGPHWSISRDEHEYPEHDVFKPERFLQSGGAEANGTSAQDE
VKGTWFAPARGSVAFGFGRRVCPGLNVAMRSLHINIACMAWAFDIAQPDG
RPERVDTFAFNSAANSHPLPFDATFTYRDPARKGVVEEENIATGELDRIA
ASRGAT

Seq. ID No: 491

>T5H_10MLEALSSLATALWAALRPDTVLLGTLAFLLFVDFLKRRHPKNY
PPGPPGLPFVGNLFQLDPEKVPLVLHQFVKKYGNVFSLDFGTVPSVLITG
LPLIKEVLVHQGQIFSNRPIVPLQEHIINNKGLIMSSGQLWKEQRRFALT
TLRNFGLGKKSLEERIQEEASYLIQTIREENGQPFDPHLTINNAVSNIIC
SITFGERFDYQDDQFQELLRMLDEILNLQTSMCCQLYNVFPRIMNFLPGP
HQALFSNMEKMKMFVARMIENHKRDWNPAEARDFIDAYLQEIEKHKGDAT
SSFQEENLIYNTLDLFLAGTETTSTSLRWGLLFMALNPEIQEKVQAEIDR
VLGQSQQPSMAARESMPYTNAVIHEVLRMGNIIPLNVPREVAVDTTLAGY
HLPKGTMVMTNLTALHRDPTEWATPDTFNPEHFLENGQFKKRESFLPFSI
GKRMCLGEQLARTELFIFFTSLLQKFTFRPPENEQLSLKFRVSLTLAPVS
HRLCAVPRG

Seq. ID No: 492

>T5H_11MKTPPQSSCPFHAVGRPPTPPRSSAGRWPPGPESGLTGWGLLK
LMSRDLMGTLAGWQREFGDLVHVRTWPEHQVIVSDPQLARELLVNQADAL
QRWERALTVYRRVHGHSVLIAEGQAWREKRQALQPDFTRKSVQAFSPSIV
EAARRAFEQWPARHAAWPIESELTSVTMEVILRMMFSSGVGSEAQQAEEA
VHTLMVASTEELWRPASLPDWVPWQRKRRRARLLMNGLIERHLQARLAMP
QDAWPEDLLSRLLRLHLQQPQSWPLQAVRDECKTAFLAGHETVATSLTWW
AWCMASHPEIQERAREEALAALSGGGQADPAALQYVNQTLLETMRLYPAV
PLLMSRRALKPVTLGDWTFPAKTVFMVPMQLMQHDERWFPEPRSYRPERF
GPDAARPQQGAYLPFGGGPRVCLGQHLAMAEMALVAAQLLLRYRLSAPEG
AEPPRPVFHVSQRPSQPLTLGIARI

Seq. ID No: 493

>T5H_12MKLAGKRFRLPPGPSGAPIVGNWLQVGDDLNHRNLMGLAKRFG
EVFLLRMGVRNLVVVSSPELAKEVLHTQGVEFGSRTRNVVFDIFTGKGQD
MVFTVYGDHWRKMRRIMTVPFFTNKVVAQNRVGWEEEARLVVEDLRADPA
AATKGVVVRRRLQLMMYNDMFRIMFDRRFETVADPLFNQLKALNAERSIL
SQSFDYNYGDFIPVLRPFLRRYLNRCTNLKTKRMKVFEDHFVQQRKEALE
KTGEIKCAMDHILEAERKGEINHDNVLYIVENINVAAIETTLWSIEWGLA
ELVNHPEIQQKLREEIVAVLGPGTPVTEPDLERLPYLQSVVKETLRLRMA
IPLLVPHMNLSDAKLAGYDIPAESKILVNAWFLANDPKRWVRADEFRPER
FLEEEKSVEAHGNDFRFVPFGVGRRSCPGIILALPIIGITLGRLVQNFEL
LPPPGQDKIDTTEKPGQFSNQILKHATIVCKPLEA

Seq. ID No: 494

>T5H_13MHTDTPDTTADQPLRRIKDLPGPRPLPLIGNGHQIKPQRIHQH
VERWSLQYGPLMRMYFGATPILVVADHEMVGAVLRDRPDGFRRPSISATI
SNEMGGIPGLFLAEGADWRNQRRMVMAGFAPTAIKAYFPALVAVALRLRR
RWQAAASARKAIDLESDLKRYTVDIIAGLAFGSDVNTLESGEDVIQRHLD
DILPAVARRSLALVPYWRYVKLPADRRLDRSVAVLRTAVQDLIGQARQRM
LDNPARRERPPNLLEAMIAAADQSGSGVTDLNVAGNVTNMLLAGEDTTAN
TISWMIYLLQRHPHTLQKARDEVRRNAPDAARFTIEQLDSLDYLGACANE
AMRLKPVAPYLPLEALRDTVIGDVAVPAGTMIWCVLRHDSVAEKHFPDPL
LFDPQRWLQADGKPNSDKRVTMPFGAGLRTCPGRYLALLEIKIAMAMLLG
SFDIAGVDTPDGKEAQELMGFVMSPVGLSLRLE

Seq. ID No: 495

>T5H_14MLMKTLMASLQWLKESFQPFMLLFASIFLAVLLKFFFKEKSRK
RSNLPPSPPKLPIIGNLHQLGNMPHLSLHNLAKKYGSIIFLQLGEIPTVV
VSSARLAKEVMKTHDLALSSRPQIFSAKHLFYNCTDVVFSPYGAYWRHIR
KICILELLSVKRVQSYSFVREEEVARLVRRVAEFYPGTTDLTKILGLYAN
DVLCRVAFGRDFSGGGEYDQHGFQKMLEEYQELLGGFSLGDFFPSMEFVH
SLTGMKSRLQDTFRRFDQLFDLFLTEHRDPKRETEEHKDLVDVLLDLQKN
AYDEMPLTTDNIKAIILDMFAAGTDTTFITLDWGMTELIMNPEVMERAQA
EVRSVVGDREVVLQSDLPQLHYIKAVIKEIFRLHPPAPVLVPRESMEDVS
IDGYNIPSKTRFFVNAWAIGRDPESWENPNAFEPERFMDSTIDFKGQHFE
LIPFGAGRRSCPAIAFGEATIELALAQLLHSFDWELPPGTTPKDLDMSEV
FGITMHRIAHLIVIAKPRFPVGQNK

Seq. ID No: 496

>T5H_15MPKQKKRLPPGPPTLPIIGNMHQLGELAHKSLSELSKKYGPIM
LLKIGSKTIINISSAEAARQVLKVHDLDCCSRPVSSTAGRLTYNFKDIVF
APYGDYWREMRKICALELLSVARVQSYRFIREEEVASLVNSISQSASSAT
PVDLSEKMLALTVNILCRTAFGKSFRGSGLDNGKLREVVHEAEVMFASFS
ATEFFPYVGWIIDRLSGRIRRLEKIFRGLDDFLQQAIDLHLKPKKTEQDH
EDLIDVLLKIERDQQTNTGAPPFNKDNIKAILFDMFLGGSNTAAVTMLWA
MAELARNPRAMKKAQDEVRNVVGNRGKVTESDITHLHYLKMTIKETFRLH
PPAAILLPRQTMAEVKIGGYDIGPNSLLQVNAWALGRDPEYWMNPEEFYP
ERFVDSSIDYKGQHFELLPFGSGRRGCPGMHMGTTTVELALANLLYCFDW
KLPSGLKEEDINMDESTGPGLTQKRTTLKLVPVKLF

Seq. ID No: 497

>T5H_16MKLLLDRTRTNGYLPPSPPKLPIIGNLHQLGKMPHISLCERAQ
KLGPIMFLQLGEVPTVVISSAAMAKEVMKTHDLAFSSRPQLYSAKWLFYN
CTNIVFSPYGAYWRHVRKICILELLSTKRVQSYGFIRQEEVSRLLHRIAD
SCSKPINLSKLLGLYANDVLCRAVLGRNFSEGGDYDMHGFQSMLKEYQEL
LGGFSIGDFFPSKEFVHLLTGHKRRLQNTFKRFDNFFQQVVREHLDPERN
YEGEKDILDVLLDIQKNGSSEMPLTLDNVKAILLDMFAAGTDTSFIVLDW
GMTELIMNPKVMKKAQAEIRRVVGERQVVLENDLPQLHYLKAVIKEIFRL
HPPVPVLVPRESIQDVTIEGYNIPAKTRVFINVWAIGRDPESWKNPETFD
PERFVGSTIDFKGQDFELLPFGAGRRGCPGITFGAVTVELALAQLLHSFD
WKLPLGVEAKDLDLTEAFGISMPKTSDLIVVAKPCFA

Seq. ID No: 498

>T5H_17MRMDGNSTTMFPLLITVIMLLASVLFYIFNRWTHRYSKSGILP
PSPPKLPLLGHLHLLSDQPHVALSRLAQKYGPIMYLELGQVPTVVVSSAS
LAREVLKTHDHVFCNRPQTIAAQYISFGCSDVTFSPYGPYWRQVRKICVT
ELLTLRRVNSFQLIREEETNRLLTAVGAHSGSEVNLTKLFFNLANDTLCR
AAFGTRFMSESTQLERQREGKRLEDILIETVKLLSGFYVGEFFPRWGWIN
SVSGFKRRLERNLADLRSVGDEIIQEHIKKRGRGNEEEDFVDVLLRVQRQ
QDLQVPITDDNVTALVMDLFVAGTDTTSSTLEWTMTEMARHPEVMKKAQA
EVRSMSPEGGTLDESHLRHLHYLKAVIKEALRLHPPIPLLLPRESMDKCA
IDGYEIPAKTRVLINNFALGRDPDSWDDPLRYNPARFMGGDEHKIDFKGE
DFRFVPFGGGRRGCPGYSLGLATVELTLARLLYHFDWKLPPGVEAEKIDL
TEIFGLATRKKTPLLLIPTARKAPPHE

Seq. ID No: 499

>T5H_18MELTMASTMSLALLVLSAAYVLVALRRSRSSSSKPRRLPPSPP
GWPVIGHLHLMSGMPHHALAELARTMRAPLFRMRLGSVPAVVISKPDLAR
AALTTNDAALASRPHLLSGQFLSFGCSDVTFAPAGPYHRMARRVVVSELL
SARRVATYGAVRVKELRRLLAHLTKNTSPAKPVDLSECFLNLANDVLCRV
AFGRRFPHGEGDKLGAVLAEAQDLFAGFTIGDFFPELEPVASTVTGLRRR
LKKCLADLREACDVIVDEHISGNRQRIPGDRDEDFVDVLLRVQKSPDLEV
PLTDDNLKALVLDMFVAGTDTTFATLEWVMTELVRHPRILKKAQEEVRRV
VGDSGRVEESHLGELHYMRAIIKETFRLHPAVPLLVPRESVAPCTLGGYD
IPARTRVFINTFAMGRDPEIWDNPLEYSPERFESAGGGGEIDLKDPDYKL
LPFGGGRRGCPGYTFALATVQVSLASLLYHFEWALPAGVRAEDVNLDETF
GLATRKKEPLFVAVRKSDAYEFKGEELSEV

Seq. ID No: 500

>T5H_19MPLSDSTISLLLAVLPISGIIFALYNQYQIWLKSPIRGLPYPP
GPPLLLGNANRAVQSRPWLTYTEWAKQYGDIIYVNIYGEHTVILNNLEDV
MELFEQRSRVYSSRQNNPYIELMGWQFNAGLLPYGDLWRRHRKLLQQCFR
RKISTQYEPIQIAKTHNLLNDLLQTPSDFIEHIKRNSSAMIMSILYGQDI
SDEMSAQFVSVAEESVKALGKCLRPGTYLVSYIPMLRYLPAWFPGAEFQR
QAAEVKKLTTKMKDEPIDFVGKGLLHGTASASLVADLLENCYVQREYDVI
KDVAATVFAAGADTSVAALESFFLAMSLFPEAQKKAQAEMDRVIGNKRLP
TTDDRPLLPYLEAVYRELMRWAPVVPLNAAHTTIADDIYKGYYIPKGTAV
YANTWALTRNEEKYPNPDIFNPDRFFTETGELNDDDTVLTFGFGRRICPG
RHMASTTVWLTIASVLSNFDIKGKGTNTKDQKFTSIGEMFTDNFISRPVP
FECDIVPRKNAALLASK

Seq. ID No: 501

>T5H_20MAFETTNGILLAASLFAGVVLYLQKRKRYTLPYPPGPKKHFLL
GNLLDVPTTFAWKRYAEWGKTFDSDVLHLSVAGSHFIILNSFKAANDLFE
KRSSIYSSRAQMIMFSELIGWDWLMSGMVYGEPWRERRKAFQQYFHVGNA
HLYEPVQMQAVRKMLPRLLKEPEDFLSITRHALGSMALTLAYGLDIQEKN
DPYLRVSEAAVKSIGEVAIPGAFLVDMIPALKYVPEFFPGAGFKKKARIW
RKVQENMREIPFAATLKNIASGSAKVSFTSTCLENLDESRDVDHQRTIIK
DTAGNMFAAATDTTISAIHTFFVAMLCFPEVQKKAQQEIDRVLQGRLPEF
SDEADLPYLSALVKETLRWEPSTPIGVPHYSSEDDVYNGYHIPKGSLVIG
NAWAMLHNEEDYPEPSLFKPERFIKDGKLNPNVRDPAEMAFGFGRRLCPG
NHIAISALWLTAATVLATFNITEAIDDDGRPIKPCVEYESALICHPLPFK
CTIKPRSKECTMLIQAAADSY

Seq. ID No: 502

>T5H_21MIIDSSNSEGNSEGQYTIDGPKAKGLRRMFRIFHLILQPTKYM
ESSVQRYGSMFQIGSEGASPLVYVGEPEVVKEIFALDGDQVVTGQGNGVL
ETMVGKHSILLLDGDPHRQQRKLLMPPFHGEQLRAYAHLICDITRQISAQ
WQPGQTIVARPPIQNLTLGVILQAVFGVPSGERLSRLQQLMSTLLDSFAY
PISASFLFFPALQKDLGEWSPWGKFIRLREEVRSLIYAEIRDRRQQLERS
AIEQDEKLGEKLGEKTDILTLLLQARDEDGGAMSDAELHDEIVTLLLAGH
ETTASAIVWMLYWIHYLPEVQQKLRAELDALGPDPDPMAIAQLPYLTAVC
QEALRIYPITPTTFIRRLREPMTLAGYRFKAGTALMPATYIIHQRPDLYP
EPKQFRPERFLERQFAPHEFLPFGGGHRYCIGSALAMMELKLSIATLLAD
FELALLHSRPLLPARRGLTMAPPAAMKLRIKARKTNKA

Seq. ID No: 503

>T5H_22MPAPKTAPSTLPLPPGRLGLPWIGETLSFLRDPNFATKRQAQY
GSLFKSRIIGQPTVFFCGPEANAFLLSSHADCFSWRDGWPGTFQELLGES
LFLQEGETHLRNRRLLMPAFHGKALASYFSTMVALSDSYLARWEKKQQLT
WFLEFKKFTFEVASVLLVGSAPGHDETDNTIGTAESAETEAQIAQLASWF
ADLTNGLFTLPIRWGPTTYRKALRGRDRLLSYIEQEITKRRQLLARLQTD
PTAALPTDVLTLLLQTEDDEGNRLSEAEIKVQTLLMLFAGHETTTSMLTS
LVMSLAQNPDVLAKARAEQQAFPAESALTFEQIQQMPYLDQILKEVERQY
PPVGGGFRRVIKPFNFNGYHVPAGWLALYRIDAAHKDERCYTNPSDFDPD
RFSPERAEQKRYDYSLVGFGGGPRVCLGMAFAKLEMKIMAAQLLRRYHWQ
LDADQDLTMNPVPSLRPADGLKVRFSKLSFTA

Seq. ID No: 504

>T5H_23MLDMPSVKPWLTFSDWASKFGDISHLEIFGQHIVVLNSAKTAV
EMLDRKSSIYSDRPVLPMGGELVGWRNTLVLLPYGDNFREYRRNFHRVIG
SRAAMSVYHAIEEEETHKFLQRVLTKPADLSAHVRTTAGAIILRISHGYH
IQEDGDPFVSLADTAVDQFSRSTATGAFMVDLIPALAYVPEWFPGASFQR
KAREWRATLHEMVNQPYKFVQDQMAAGIAPKSFTSNLLEGRTLTEEEEHI
IKWSGASLYSGGADTTVSAIYGFFLAMTLYPEAQKKAQAEIDAVVGSDRL
PTFADRESLPYAEALVKEVLRWCPVVPIVVPHRVTADDIHNGYYIPKGTL
VLANAWYMLRDPSIYPDPMNFNPDRFLPSGGKEPPTDPRDICFGFGRRIC
PGMHLADASVWLSAVMSLAVFNVSKVVENGVEITPEVDPSSGTISHPKPF
KCSIKPRSAKALELIQQTPHY

Seq. ID No: 505

>T5H_24MHLPPGPRPLPFLGNLLQMNRRGLLRSFMQLQEKYGDVFTVHL
GPRPVVILCGTDTIREALVDQAEAFSGRGTVAVLHPVVQGYGVIFANGER
WKILRRFSLVTMRNFGMGKRSVEERIKEEAQCLVEELKKYKGALLNPTSI
FQSIAANIICSIVFGERFDYKDHQFLRLLDLIYQTFSLMGSLSSQVFELF
SGFLKYFPGVHKQISKNLQEILNYIDHSVEKHRATLDPNTPRDFIDTYLL
HMEKEKSNHHTEFHHQNLVISVLSLFFAGTETTSTTLRYSFLIMLKYPHV
AEKVQKEIDQVISSHRLPTLDDRIKMPYTDAVIHEIQRFADLAPIGLPHR
VTKDTMFRGYLLPKNTEVYPILSSALHDPRYFDHPDTFNPEHFLDANGTL
KKSEAFLPFSTGKRTCLGEGIARNELFIFFTALLQNFSLASPVAPEDIDL
TPINSGAGKIPSPYQINFLSRCVG

Seq. ID No: 506

>T5H_25MYLIPDFSKETWILLIILLALLAYYGIWPYRLFKKYGIPGPKP
LPFFGTFLENRNGVFEFDMECFKKFGKVWGFYDGRQPVLAIMDPVIIKAI
LVKECYTVFTNRRNFGLNGPLNSAVSIAADDQWKRIRTVLSPTFTSGKLK
QMFPIIKQYGDLLVKNIQKKVDNKEFIDMKNIFGSYSMDIVLSTSFSVNV
DSLNNPNDPFVTNGRNLFTFSFLNPLFLTTLLCPFLIPILDKLNFCFLPI
SVLNFFQDAITSIKKNRQKGIHKDRVDFLQLMVDAQANDSKGGADHGYKE
LTDTEIMAQGLIFIIAGYETTSTTLMFLAYHLATHPDVQTKLQEEIDIIL
PNKAPPTYEALMQMEYLDMVLYENLRLYPAAGRIERVCKATTEINGVTIP
KGVVTVIPAFVLHRDPELWPEPDEFRPERFSKENRETQDPYTFLPFGAGP
RNCIGMRFALINMKSVITLLLQNFSFRTCKDTPIPLQIDTRGFLKTTKPV
ILNLVPREAQKTEK

Seq. ID No: 507

>T5H_26MYDTFLEWIEKYGPVVRVNSSHSTFVIVISPEGVKEFLMSPKY
TKDNFYERIETLFGARFLGKGLVTDRDYDHWHKQRRMMDPAFSRTYLIGL
MGTFNETAEDLMDVLGDKADGKCQVGMHDMLSRVTLDVIAKAAFGMELNS
LHDDQTPFTRAISTVMKGMVETRNPLARYIPGKQAFIREVKESIKLLRET
GRECILQRRKEIQDGEDIPMDILTQILKGAEIEDGCSLEDLIDNFVTFFV
AGQETTANQLSFAVMELARNPEILTRVQTEVDEVLGSKRDIEYEDLGKLQ
YLSQVLKETLRLYPIAPGTSRALEKETVIEGVRVPPGTTLMFNSYIMGRM
EKYYHDPFIFNPDRFHPDAPKPSCAYFPFSLGPRSCIGQVFARMEAKVVM
AKLLQRFEFELVEGQSFRIMDTGTLRPMDGVICRLRPRAERKSRK

Seq. ID No: 508

>T5H_27MAARPKPATPPSPPALPVIGHLHLLTDMPHHTFADLSNSLGPL
IYLRLGQVPTIVIHSAHLAKLVLRTHDHAFANRPQLISAQYLSFGCSDVT
FSSYGAYWRQARKICVTELLSAKRVHSFRLVRKEEVDRLLDAVLTSSGKE
VDMSQMLFCLANDVLCKVAFGRRFMAEKDGKGKNLGSVLMETQALFAGFC
LGDFFPKWEWVNSMSGYRKRLLKNLKDLKEVCDEIIEEHLKKKKKKNGTE
NADDDDDYNEKEDFVDVLLRVQKREDLEVPITDDNLKALVLDMFVAGTDT
SSATLEWVFTELARHPRVMKKAQEEVRMIASGNGKVDESDLQHLHYMKAV
IKETMRLHPPVPLLVPRESMEKCALDGYEIPAKTRVLINTYAIGRDPKSW
ENPLDYDPERFMEDDIDFKDQDFRFLPFGGGRRGCPGYSFGLATIEITLA
RLLYHFDWALPHGVEADDVDLSEVFGLATRKKTALVLVPTANKDFQFRGH
DF

Seq. ID No: 509

>T5H_28MGKNKVPPGPIGLPFIGNLHQFDTLAPHIYFWELSKKYGKIFS
FKLTSNVPIIVVSSAKLAKEVLKTQDLVFCSRPSLVGQQKLSYNGHDIGF
APYNDYWREMRKICVLHLFSLKKVQLFSPIREDEVSRMIKKIYQQAVNSQ
VTNLSNLMISLNSTIICRVAFGVRFDEEAHERKRFNYILAEAQAMFAGFF
MSDFFPSLSWIDKLTGMIDRLEKNFKDLDEFYEELIEQHYNPNRPKSMEG
DFIDILLQLKKDQLTPIDLSLEDIKGILMNVLLAGSDTSSSVIIWAMTIL
IKNPKAMKKVQEEIRNLIGNKGIVNEDDIQNMHYLKAVIKETLRLFPPAP
LLIPRESMKISTLEGYEFQPRTIVYVNAWAIARDPEIWENPEEFMPERFL
NSNIDFKGQDYELIPFGAGRRGCPGLALGVASVELALSNLLYAFDWELPY
GLKKEDIDINGKPGITVNKKNDLCLIPKKYF

Seq. ID No: 510

>T5H_29MKLTGKRYRLPPGPAGAPVVGNWLQVGDDLNHRNLMSLAKRFG
DIFLLRMGVRNLVVVSTPELAKEVLHTQGVEFGSRTRNVVFDIFTGKGQD
MVFTVYGDHWRKMRRIMTVPFFTNKVVAQNRVGWEEEARLVVEDVRKDPR
AAAEGVVIRRRLQLMMYNDMFRIMFDTRFESEQDPLFNKLKALNAERSRL
SQSFEYNYGDFIPVLRPFLRGYLNRCHDLKTRRMKVFEDNFVQERKKVMA
QTGEIRCAMDHILEAERKGEINHDNVLYIVENINVAAIETTLWSIEWGIA
ELVNHPAIQSKLREEMDSVLGAGVPVTEPDLERLPYLQAIVKETLRLRMA
IPLLVPHMNLNDGKLAGYDIPAESKILVNAWFLANDPKRWVRPDEFRPER
FLEEEKTVEAHGNDFRFVPFGVGRRSCPGIILALPIIGITLGRLVQNFQL
LPPPGQDKIDTTEKPGQFSNQIAKHATIVCKPLEA

Seq. ID No: 511

>T5H_30MKPRGAKYPNSLPCLPFIGSLLHLASHLAPHILFNKLQEKYGS
LYSFKMGSHYIVIVNHHEHAKEVLLKKGKTFGGRPRAVTTDLLTRNAKDI
AFADYSPTWKFHRKLVHAALSMFGEGTVAIEKIISREAASLCQTLITFQG
SPLDMAPELTRAVTNVVCALCFNARYKRCDPEFEEMLAYSKGIVDTVAKD
SLVDIFPWLQIFPNKDLEILKRSVAIRDKLLQKKLKEHKEAFCGEEVNDL
LDALLKAKLSMENNNSNISQEVGLTDDHLLMTVGDIFGAGVETTTTVLKW
AVAYLLHYPKVQAKIQEELDVKVGFGRHPVLSDRRILPYLDATISEVLRI
RPVAPLLIPHVALHESSIGEYTIPQDARVVINLWSLHHDPNEWENPEEFI
PDRFLDENGNHLYTPSQSYLPFGAGIRVCLGEALAKMEIFLFLSWILQRF
TLEVPAGDSLPDLDGKFGVVLQVKKFRVTAKLREVWKNIDLTT

Seq. ID No: 512

>TAT2MTEDFISSVKRSNEELKERKSNFGFVEYKSKQLTSSSSHNSNSSH
HDDDNQHGKRNIFQRCVDSFKSPLDGSFDTSNLKRTLKPRHLIMIAIGGS
IGTGLFVGSGKAIAEGGPLGVVIGWAIAGSQIIGTIHGLGEITVRFPVVG
AFANYGTRFLDPSISFVVSTIYVLQWFFVLPLEIIAAAMTVQYWNSSIDP
VIWVAIFYAVIVSINLFGVRGFGEAEFAFSTIKAITVCGFIILCVVLICG
GGPDHEFIGAKYWHDPGCLANGFPGVLSVLVVASYSLGGIEMTCLASGET
DPKGLPSAIKQVFWRILFFFLISLTLVGFLVPYTNQNLLGGSSVDNSPFV
IAIKLHHIKALPSIVNAVILISVLSVGNSCIFASSRTLCSMAHQGLIPWW
FGYIDRAGRPLVGIMANSLFGLLAFLVKSGSMSEVFNWLMAIAGLATCIV
WLSINLSHIRFRLAMKAQGKSLDELEFVSAVGIWGSAYSALINCLILIAQ
FYCSLWPIGGWTSGKERAKIFFQNYLCALIMLFIFIVHKIYYKCQTGKWW
GVKALKDIDLETDRKDIDIEIVKQEIAEKKMYLDSRPWYVRQFHFWC

Seq. ID No: 513

>TMO_1MSTLADQALHNNNVGPIIRAGDLVEPVIETAEIDNPGKEITVED
RRAYVRIAAEGELILTRKTLEEQLGRPFNMQELEINLASFAGQIQADEDQ
IRFYFDKTMGGGSGEGRGSLLTCGDVEENPGPMFNIQSDDLLHHFEADSN
DTLLSAALRAELVFPYECNSGGCGACKIELLEGEVSNLWPDAPGLAAREL
RKNRFLACQCKPLSDLKIKVINRAEGRASHPPKRFSTRVVSKRFLSDEMF
ELRLEAEQKVVFSPGQYFMVDVPELGTRAYSAANPVDGNTLTLIVKAVPN
GKVSCALANETIETLQLDGPYGLSVLKTADETQSVFIAGGSGIAPMVSMV
NTLIAQGYEKPITVFYGSRLEAELEAAETLFGWKENLKLINVSSSVVGNS
EKKYPTGYVHEIIPEYMEGLLGAEFYLCGPPQMINSVQKLLMIENKVPFE
AIHFDRFF

Seq. ID No: 514

>TMO_2MAMHPRKDWYELTRATNWTPSYVTEEQLFPERMSGHMGIPLEKW
ESYDEPYKTSYPEYVSIQREKDAGAYSVKAALERAKIYENSDPGWISTLK
SHYGAIAVGEYAAVTGEGRMARFSKAPGNRNMATFGMMDELRHGQLQLFF
PHEYCKKDRQFDWAWRAYHSNEWAAIAAKHFFDDIITGRDAISVAIMLTF
SFETGFTNMQFLGLAADAAEAGDYTFANLISSIQTDESRHAQQGGPALQL
LIENGKREEAQKKVDMAIWRAWRLFAVLTGPVMDYYTPLEDRSQSFKEFM
YEWIIGQFERSLIDLGLDKPWYWDLFLKDIDELHHSYHMGVWYWRTTAWW
NPAAGVTPEERDWLEEKYPGWNKRWGRCWDVITENVLNDRMDLVSPETLP
SVCNMSQIPLVGVPGDDWNIEVFSLEHNGRLYHFGSEVDRWVFQQDPVQY
QNHMNIVDRFLAGQIQPMTLEGALKYMGFQSIEEMGKDAHDFAWADKCKP
AMKKSAGGGSGEGRGSLLTCGDVEENPGPMSFEKICSLDDIWVGEMETFE
TSDGTEVLIVNSEEHGVKAYQAMCPHQEILLSEGSYEGGVITCRAHLWTF
NDGTGHGINPDDCCLAEYPVEVKGDDIYVSTKGILPNKAHS

Seq. ID No: 515

>TMO_3MSFTKVCSVGDIWEGEMEPFTVDGHEILLVGVEGGGIKAFQGIC
PHQDIALSEGKFDGKKLICRAHLWQFDASNGKGINPDDCALAEYPVKVDG
DDVYVQTAGVEALFAHSGGGSGEGRGSLLTCGDVEENPGPMALLNRMDWY
DLARTTNWSPKYVTESELFPPELSGDHGIPMEKWETYDEPYKQTYPEYVK
VQREKDAGAYSVKAALERSQIYERSDPGWLTVMKQHYGAIALGEYAASSA
EARMMRFSKAPGMRNMATLGSMDEIRHGQIQLYFPHEHVSKDRQFDWAAK
AFHTNEWAAIAARHFFDDIMMTRDAISVAIMLTFSFETGFTNMQFLGLAA
DAAEAGDHTFASLISSVQTDESRHAQIGGPTLQILIENGKKAEAQKKVDI
AFWRAWRLFSVLTGPVMDYYTPLEHRKQSFKEFMQEWIVAQFERALSDLG
LDKPWYWDTFLQQLDQQHHGMHLGVWYWRPTVWWNPAAGVTPAERDWLEE
KYPGWNDTWGQCWDVIIDNLVDGNIAQTYPETLPIVCNMCNLPINCTPGN
GWAVQDYPLEYNGRLYHFGSEPDRWCFEQEPERYAGHMTLVDRFLAGLVQ
PMDLGGALAYMGLAPGEIGDDAHGYSWVDIYKKMRMKKAS

Seq. ID No: 516

>TMO_4MSVASSAQAYHNNMVGPVMRAGDLALAVIEAARVDNPGKEVFVD
DKRAYVRIHTEQEMILRRETIEEELGRPFKMNDLEVDLSSFAGQIESLDD
AVRFYFTKKLGGGSGEGRGSLLTCGDVEENPGPSSNPIIHNQKDGSRFAQ
REGDTILRAALRAGVGLSYECNSGGCGGCKFELLEGEVDTLWPDAPGLSD
KDRRRGRHLACQCRARGPVSIKAATGAEYVPKVVPQRQTARLVGSTDITH
DLREFRFRSAAGASFLPGQFAMLDLPGLASARAYSMSNTANDDGEWHFQV
RRVPHGQGTHVLFDRLGVGDEIGLDGPYGVAWLRTGAPRDIVCVAGGSGL
APMVSIARGAAAAGMLKDRKLYFFYGARTPRDVCGAEMLAQLDGFGERII
YLPVVSLPGGEGEWQGETGYVHDAVARTLPGSLAGFEFYFAGPPPMTQAL
QEMLMVGHRVPFEQIHFDRFF

Seq. ID No: 517

>TPH_1MPSRLNKDEYQFYIDLDNKSTPALNEIVKCLRLDIGATVHELSR
DKKKDAVPWFPKTIQDLDKFANQILSYGAELDSDHPGFTDPVYRARRKEF
ADIAFHYKHGQPIPCVTYTEEEKKTWGTVFKELKLLYPTHACYEHNHVFP
LLEKYCGYNENNIPQLEDVSKFLQTCTGFRLRPVAGLLSSRDFLAGLAFR
VFHSTQYIRHWSKPMYTPEPDICHELLGHAPLFADPSFAQFSQEIGLASL
GAPDEYIERLATLYWFTVEFGLCKQDDKIKAYGAGLLSSFGELQYCLTDK
PELKPFEPEKTSLQKYPITEFQPVYFIAESFEDAKEKMRKFATTIPRPFS
VRYNPYTQSIEVLDNVQQLKNLADCINSEIGTLCCA

Seq. ID No: 518

>TPH_2MIEDNKENKDHSLERGRASLIFSLKNEVGGLIKALKIFQEKHVN
LLHIESRKSKRRNSEFEIFVDCDINREQLNDIFHLLKSHTNVLSVNLPDN
FTLKEDGMETVPWFPKKISDLDHCANRVLMYGSELDADHPGFKDNVYRKR
RKYFADLAMNYKHGDPIPKVEFTEEEIKTWGTVFQELNKLYPTHACREYL
KNLPLLSKYCGYREDNIPQLEDVSNFLKERTGFSIRPVAGYLSPRDFLSG
LAFRVFHCTQYVRHSSDPFYTPEPDTCHELLGHVPLLAEPSFAQFSQEIG
LASLGASEEAVQKLATCYFFTVEFGLCKQDGQLRVFGAGLLSSISELKHA
LSGHAKVKPFDPKITCKQECLITTFQDVYFVSESFEDAKEKMREFTKTIK
RPFGVKYNPYTRSIQILKDTKSITSAMNELQHDLDVVSDALAKVSRKPSI

Seq. ID No: 519

>TPH_3MSGLALDRSSQPHEVRTLEVNELDPKVFAVVEVRKDEPGVLGDV
LKVFTESSINITNIESRFKSFARDGPAFHIDFEGEAREHRVQRVLRDVKS
VPGVSQVTVMEEREVPWFPINIRDLDLTTDTLDGGTALINEDHPGFNDLA
YRQRREEIVTAAKEHRHGDRIARVQYLEHEVETWRAVYEQLRECHSRWAC
TEYLEMLPQMERFCGYAPGNIPQLADISDFLQQRTGFTLRPITGLLSARD
FLNALAFRVFYSTQYIRHHGNPFYTPEPDICHELMGHVPLFANAAFADFS
QEIGLASLAASDDDIARLAAVYWFTVEFGLVRQGGEVKAYGAGLLSSFGE
MEWSCSREPSTTCREMGSVAELQAPSIVPLDPTQAGKQAYPITTYQPLYF
CAESMQDAKAKISQFCDTLTRPFFPQYDPLTQNIRVTKAVRRARRISTVE
MQMAKQLDYFEKQ

Seq. ID No: 520

>TPH_4MAVPWFPKTIQDLDKFANQILSYGAELDSDHPGFTDPVYRTRRK
EFADIAFHYKHGQPIPRVTYTEEEKKTWGTVFKELKLLYPTHACYEHNHV
FPLLEKYCGYNENNIPQLEDVSNFLQTCTGFRLRPVAGLLSSRDFLAGLA
FRVFHSTQYIRHWSKPMYTPEPDICHELLGHAPLFADPSFAQFSQEIGLA
SLGAPDEYIERLATLYWFTIEFGLCKQDDKIKAYGAGLLSSFGELQYCLT
DKPDLKPFEPEKTSLQKYPITEFQPVYFIAESFEDAKEKVRKFATTIPRP
FSVRYNPYTQSIEVLDNVQQLKNLADCINSEIGILCCALRKLE

Seq. ID No: 521

>TPH_5MLISFTLNLVHQKKNSEFEIFLDCDSNREQLNEIFQLLRPHVNL
ITMNPQEDFSVEEDDMESVPWFPIKISDLDKSANRVLMYGSDLDADHPGF
KDNVYRRRRKYFADVAMNYKYGDPIPHIEFTEEEVKTWGTVFRELNKLHQ
THACREYLKNLPLLVKHCGYREDNIPQLEDVSRFLKERSGFTIRPVAGYL
SPRDFLAGLAFRVFHCTQYVRHSSDPLYTPEPDTCHELLGHVPLLAEPSF
AQFSQEIGLASLGASDEAVQKLATCYFFTVEFGLCKQEGKLKVYGAGLLS
SISELKHSLSGNANVKPFDPMVTCSQECIITSFQEVYFYSESFEEAKEKM
REFAKTIKRPFGLKYNPYTQSVQMLKDTQSITTLVSELRHELDIISDALN
KMNKQLGV

Seq. ID No: 522

>TPH_6MHSPEPDCCHELLGHVPMLADKTFAQFSQDIGLASLGVTDEEIE
KLSTLYWFTVEFGLCKQDGEVKAYGAGLLSSYGELLHALSDKPEVRPFDP
DEAAIQPYQDQNYQPVYFVSESFTDAKEKLRNYASRIKRPFAARYDPYTV
SIEVLDSPGQIQSSLEELKDELQTLTTALNILS

Seq. ID No: 523

>TPH_7MMISTESDLRRQLDENVRSEADESTKEECPYINAVQSHHQNVQE
MSIIISLVKNMNDMKSIISIFTDRNINILHIESRLGRLNMKKHTEKSEFE
PLELLVHVEVPCIEVERLLEELKSFSSYRIVQNPLMNLPEAKNPTLDDKV
PWFPRHISDLDKVSNSVLMYGKELDADHPGFKDKEYRKRRMMFADIALNY
KWGQQIPIVEYTEIEKTTWGRIYRELTRLYKTSACHEFQKNLGLLQDKAG
YNEFDLPQLQVVSDFLKARTGFCLRPVAGYLSARDFLSGLAFRVFYCTQY
IRHQADPFYTPEPDCCHELLGHVPMLADPKFARFSQEIGLASLGTSDEEI
KKLATCYFFTIEFGLCRQDNQLKAYGAGLLSSVAELQHALSDKAVIKPFI
PMKVINEECLVTTFQNGYFETSSFEDATRQMREFVRTIKRPFDVHYNPYT
QSIEIIKTPKSVAKLVQDLQFELTAINESLLKMNKEIRSQQFTTNKIVTE
NRSS

Seq. ID No: 524

>TrpHalo_1MSTASKNIDITRFPKKYDAATKDSDFYDVVIVGAGPGGST
TAYYLAKEGKKVLLLEKKKFPRDKICGDAICKLAIEMLMDMGVYEGLVRE
KKARVAHNGGLVSPSGLSFIGNTYLKPGEIPAAAACKRMVLDEAIAKAAI
GAGAELKENSPVTDAVFDSSTGLWTISIEGSDVKHMGRVLVCADGAPSKL
ATQLGIVKQAPQGVCSRAYIKEGTHRFRADGVVFYPRNILPAYAALFRHI
DDTVAYCTYILPFNPKVTTDDLSYWHHRLLEEDPSISQAVGKNADMERMK
AWGLRMGGEPVTYGNHVLVVGDAAGMIDPLTGEGIHHAMDGGRIAAHFLC
EAIAVGNFDKEVMKEYQNRWLYTFGNDYKWSQAICHFLYRFPIFIDATAA
AAQRRGNNFLALYADIMTGRIPKANIFRPDISLPIAFEVLVLLWKMMFTG
GGGNNKMKSQ

Seq. ID No: 525

>TrpHalo_2MSTASKNIDITRFPKKYDAATKDSDFYDVVIVGAGPGGST
TAYYLAKEGKKVLLLEKKKFPRDKICGDAICKLAIEMLMDMGVYEGLVRE
KKARVAHNGGLVSPSGLSFIGNTYLKPGEIPAAAACKRMVLDEAIAKAAI
GAGAELKENSPVTDAVFDSSTGLWTISIEGSDVKHMGRVLVCADGAPSRL
AMQLGIVKGTPKCVCSRAYIKGGTHRFKEDGMVFYVPSILPGYVALLRHI
DDQLTYCTYILPGNPRATTKDLSYWHHRLLEEDPNISQAVGKNAELEKMK
AWDLRVGGEPVTYGNHVLVVGDAAGMIDPLTGEGIHHAMDGGRIAAHFLC
EAIAVGNFDKEVMKEYQNRWLKAFGNDFRWSQAIGNFLYRYPIFIDATAA
VAEKKGDRFLARWADIMAGRIPKISVLRPQFLLAVGFQALLLFYKKIFKG
GYGKKTKIL

Seq. ID No: 526

>TrpHalo_3MSSLIAPKVDTIDITRFPKKYDPAAEDSDFYDVVIVGAGP
GGSTTAYYLAKKGKKVLLLEKKKFPRDKICGDAICKTAIEILMDMGVYGG
LIREQKAYMIDYGGLVSPSGLSFVGHTHELFGEIPGAVVCKRVVLDKVIS
RTAQSAGAELLENSPVTDAVFDSSTGLWTISIEGSDVKHMGRVLVCADGA
PSRLAMQLGIVKGTPKCVCSRAYIKGGTHRFKEDGMVFYVPSILPGYVAL
LRHIDDQLTYCTYILPGNPRATTKDLSYWHHRLLEEDPNISQAVGKNAEL
EKMKAWDLRVGGEPVTYGNHVLVVGDAAGMIDPLTGEGIHHAMDGGRIAA
HFLCEAIAVGNFDKEVMKEYQNRWLKAFGNDFRWSQAIGNFLYRYPIFID
ATAAVAEKKGDRFLARWADIMAGRIPKISVLRPQFLLAVGFQALLLFYKK
IFKGGYGKKTKIL

Seq. ID No: 527

>TrpHalo_4MSGKIDKILIVGGGTAGWMAASYLGKALQGTADITLLQAP
DIPTLGVGEATIPNLQTAFFDFLGIPEDEWMRECNASYKVAIKFINWRTA
GEGTSEARELDGGPDHFYHSFGLLKYHEQIPLSHYWFDRSYRGKTVEPFD
YACYKEPVILDANRSPRRLDGSKVTNYAWHFDAHLVADFLRRFATEKLGV
RHVEDRVEHVQRDANGNIESVRTATGRVFDADLFVDCSGFRGLLINKAME
EPFLDMSDHLLNDSAVATQVPHDDDANGVEPFTSAIAMKSGWTWKIPMLG
RFGTGYVYSSRFATEDEAVREFCEMWHLDPETQPLNRIRFRVGRNRRAWV
GNCVSIGTSSCFVEPLESTGIYFVYAALYQLVKHFPDKSLNPVLTARFNR
EIETMFDDTRDFIQAHFYFSPRTDTPFWRANKELRLADGMQEKIDMYRAG
MAINAPASDDAQLYYGNFEEEFRNFWNNSNYYCVLAGLGLVPDAPSPRLA
HMPQATESVDEVFGAVKDRQRNLLETLPSLHEFLRQQHGR

Seq. ID No: 528

>TrpHalo_5MDEIDDPRIRSVVIVGGGTAGWMTAAALVQHFRTAPLKIT
VVESSDIGTIGVGEATIPTIRRFYGQLGLRDDDVMRATQATCKLGIRFLD
WSGPGSDFIHPFGLYGQDVKGIGFHHYWLKQRRAGDAAPLAAYSLGAALA
AGGKFTLPSPHPPSQLSVFDWALHLDAGLFAQHLRAYAEAGGCARIDARI
RSVELRPEDGFVRALTLDDGREVEGDLFVDCSGFKGLVIGEALGVGFEDW
GRWLPCDAAYAVQSENRPGDAPAPFTRVTARSAGWQWGIPLRHRAGNGLV
FSSAHLSDDQALAELMPHLLGDPLTEPRRIPFRPGRRSQAWAKNCVAIGL
SSGFLEPLESTSIALIETGIERLKALFPDRRFAQPILDEFNDQTAREMER
VRDFIILHYKLNRRTDTDFWRDCREMPVPETLERKIALWTARGQFVRYRW
EMFHPASWLAIYDGFGLYPDHHDPAVDAMDPAYLARSLAEMRANIADLVA
RTPEHAQFLAGLDPAASAA

Seq. ID No: 529

>TrpHalo_6MIRSVVIVGGGTAGWMTASYLKAAFDDRIDVTLVESGNVR
RIGVGEATFSTVRHFFDYLGLDEREWLPRCAGGYKLGIRFENWSEPGEYF
YHPFERLRVVDGFNMAEWWLAVGDRRTSFSEACYLTHRLCEAKRAPRMLD
GSLFASQVDESLGRSTLAEQRAQFPYAYHFDADEVARYLSEYAIARGVRH
VVDDVQHVGQDERGWISGVHTKQHGEISGDLFVDCTGFRGLLINQTLGGR
FQSFSDVLPNNRAVALRVPRENDEDMRPYTTATAMSAGWMWTIPLFKRDG
NGYVYSDEFISPEEAERELRSTVAPGRDDLEANHIQMRIGRNERTWINNC
VAVGLSAAFVEPLESTGIFFIQHAIEQLVKHFPGERWDPVLISAYNERMA
HMVDGVKEFLVLHYKGAQREDTPYWKAAKTRAMPDGLARKLELSASHLLD
EQTIYPYYHGFETYSWITMNLGLGIVPERPRPALLHMDPAPALAEFERLR
REGDELIAALPSCYEYLASIQ

Seq. ID No: 530

>TrpHalo_7MLESIVVVGGGTSGWMTASYLSAAFGERISVTVVESARVG
TIGVGEATFSTVRHFFEYLGLSEETWMPACNATYKLGIRFENWRAPGHHF
YHPFERQRVVDGFTLPDWWLADGGATERFDKECFLVGTLCDTMRSPRHMD
GALFEGDLTDRPAGRSTLAEQGTQFPYAYHFDAALLADFLRDYAVARGVL
HVVDDVVHVARDERGWISHVATRGSGDLAGDLFVDCTGFRGLLINDALDE
PFESYQDTLPNDSAVALRVPVDMEREGLRPCTTSTAQAAGWIWTIPLFGR
VGTGYVYARDYCTPEEAERTLRRFVGPAADDLEANHIRMRIGRSRRSWVN
NCVAVGLSSGFVEPLESTGIFFIQHAIEQLVKHFPDADWDPALRSAYNTL
VNRCMDGVREFLVLHYYGAARADNEYWRDTKTRKIPDSLAERVEQWRTKL
PHPESVYPHYHGFEAYSYVCMVLGLGGIPLKPSPALRMLDPSAAQREFRL
LATQAEDLRRTLPSQYAYFAQFR

Seq. ID No: 531

>TrpHalo_8MNKPIKNIVIVGGGTAGWMAASYLVRALQQQANITLIESA
AIPRIGVGEATIPSLQKVFFDFLGIPEREWMPQVNGAFKAAIKFVNWRKS
PDPSRDDHFYHLFGNVPNCDGVPLTHYWLRKREQGFQQPMEYACYPQPGA
LDGKLAPCLSDGTRQMSHAWHFDAHLVADFLKRWAVERGVNRVVDEVVDV
RLNNRGYISNLLTKEGRTLEADLFIDCSGMRGLLINQALKEPFIDMSDYL
LCDSAVASAVPNDDARDGVEPYTSSIAMNSGWTWKIPMLGRFGSGYVFSS
HFTSRDQATADFLKLWGLSDNQPLNQIKFRVGRNKRAWVNNCVSIGLSSC
FLEPLESTGIYFIYAALYQLVKHFPDTSFDPRLSDAFNAEIVHMFDDCRD
FVQAHYFTTSRDDTPFWLANRHDLRLSDAIKEKVQRYKAGLPLTTTSFDD
STYYETFDYEFKNFWLNGNYYCIFAGLGMLPDRSLPLLQHRPESIEKAEA
MFASIRREAERLRTSLPTNYDYLRSLRDGDAGLSRGQRGPKLAAQESL

Seq. ID No: 532

>TrpM_1MSPVALSPKRVDIVDIRGNDMQYSLVNEIHKGLNPPNGTRRSL
PTMLLYDSEGLKLFEKITYVDEYYLTNAEIEVLEKHSRRLVEKIPSNAQL
LELGSGNLRKIEILLREFERVGKPVDYYALDLSLSELERTFSNVSLEEYK
SVGFHGLHGTYDDAHTWLSDPKNRERPTVVLSMGSSLGNFSPPDAAAFLA
GFATLLKPSDFMVIGLDACEDPDRVYKAYNDSAGITRKFYENGLANANKT
LGHEVFRPDEWEVVTEYDAVNGRHQVFYVPTKDVSVGDVLLRRGEKIIFA
EAFKYGCQAREKLWHDAGLIEAAEFGSGSEDYRTYI*

Seq. ID No: 533

>TrpM_10MLGPVPSPSPVPIPPGSRPGASPGLEATIPIIDIRSTAHSVT
VAALEDGIRANVLSGFTKPYNEKELPNLLLYNEEGLRLFEQITYQPDYYL
TRLEIDILSRHAHQIANSVPDGAILLELGAGALRKTALILDALEAQGKDV
TYFALDLDKPELLRTLAEVKGRYTHVSLAGLWGTYDDGCTWLKQVKDRPR
IILWLGSSVGNMSRKEAGQFIRTFGDILAPRDRFIVAIDSKNHKLNDIRA
AYDDRAGVTRRFALNALGNINDLFNADVVDVSSFDYNPYYNEVQGRNEAY
FRCLKDTQVRIPSETPILVHEGEYIRFAFSHKYDRVERQVLWTAAGAYPV
QEWMSQDGDYALTMLSWSS*

Seq. ID No: 534

>TrpM_11MTYSIVDIRKTDTCLKNSIINGINQSTKSIPAIVLYDELGLQ
YYEKVTYLKEYYLTEAEIDILKNKADQISDYIPEGSSLIELGSGALRKTR
LLLDSIEKQKKKVIYYALDLMEGELKRTLSSLGKFQYVKLVGLWGVYEDG
IDYASNLPGDSHKTILWMGSSIGNFNRDEAANFVKTIQDKAMNPGDLFLI
GIDRRKNPDKITAAYNDPKGINAKFIMNGLNHVNAIFDQPIFDSNNFEHV
TMYNDDVGRHEAYCKVKNDTTLEFKESKDNPKTIIKLNKNELINIGYSHK
YNKAETDALFDFSLLSYMESWTDSQSLYDLHLVYKSPFHFTRKFDSHK*

Seq. ID No: 535

>TrpM_12MSKDVQVLDIRASPQSKGSIPNLRTAILDGLQKAPGMRTLPS
EILYDDRGLKIYNDCIRSWSEWYYPISAETEILEINGKDIARVFSTSDRG
EAVLIELGAGSLDKTSKILVSLSETVQNVSDSQPPITYYALDLERSELQR
TLSELQKNIGEKIAGKIATKGMWGTYDDGIRSVENNELHLDAAVPVHFLF
LGGTIGNFSKGEGDVTFLRNLPLNAQRGDTILLGIDREKSKEIIERAYNF
PAAREWIMNGLNVSGHLLSGDKDLFQLDNWDRYAMYDEKLGRLEAGYRSK
IDQIIEVTANYSIPFKKDESVMAIFSNKYTDDELNFLISKANLKTINSWV
DHKALYYIFSLRKV*

Seq. ID No: 536

>TrpM_13MPRIQVLDIRGSKESVGSTPHLRAAILEGLLKPPGSRTLPSE
TLYDEVGLKMYNDGMKAWAEWYYPVEAERQILERYGRDIAKLFTTSAKGK
AVLIELGAGSLDKTSQVLLSAAEITRTTGPMNNIAYYALDLERGELERTI
GRLQEVIGDQIAGKISTAGMWGTYDDGIRVIEKNELELEPDIPVHILFLG
GTIGNFSKQDGDVAFLKSLPLDHKRGDTLLVGMDRHKSADAIERSYGFAA
AKDWIMNGLKVSGRVLTGDEGLFEIGNWERYAKYNEELGRYEAGYKSQKE
HALKISEGVDITFLKDEVVLVMFSNKYTDAEMDSVVDSAGLVKNGSWMDE
KAQYCLLSLRANNGPV*

Seq. ID No: 537

>TrpM_14MSQIEVLDIRGSKEATGSTPHLRAEILQGLSKSPGHRTIPGE
TLFDETGLKMYDEGMKTWRKWYYPFEAEKEILEVRGLEIAKLLKTSSKGE
AVLIELGAGSLEKTSQILLSAAQIAETADNSTTNPITYYALDLEHRELER
TLAALQDAIGPRIAGKITTKGMWGTYEDGIRVVERNDLKFPSDVPLHILF
LGGTIGNFSKADGDIAFLKSLPLNRKRGDTLLLGVDRAKAVELIERAYGF
AAATGWIMNGLKVSGRVLTGDEELFESGNWERYSKYNEELGRYEAGYKSR
KDQTIKVAKDVDIVFSKDEVILVTYSNKYTDAEIKTVFDGAGLEIVESWM
DKKAQYCLFLLKA*

Seq. ID No: 538

>TrpM_2MTLSLANYLAADSAAEALRRDVRTGLTATPKSLPPKWFYDAVG
SDLFDQITRLPEYYPTRTEAQILRTRSAEIIAAAGADTLVELGSGTSEKT
RMLLDAMRDADLLRRFIPFDVDAGVLRSAGAAIGAEYPGIEIDAVCGDFE
EHLGKIPRVGRRLVVFLGSTIGNLTPQPRAEFLATLADTLQPGDSLLLGT
DLVKDTGRLVRAYDDAAGVTAAFNRNVLAVVNRELSADFDLDAFEHIAKW
NDDEERIEVWLRARTAQHVRIPALDLEIDFAAGEQMLTAVSCKFRPDSVA
AELAEAGLRQTHWWTDPAGDFGLSLAVR*

Seq. ID No: 539

>TrpM_3MTLSLANYLAADSAAEALRRDVRAGLTAAPKSLPPKWFYDAVG
SDLFDQITRLPEYYPTRTEAQILRTRSAEIIAAAGADTLVELGSGTSEKT
RMLLDAMRDAELLRRFIPFDVDAGVLRSAGAAIGAEYPGIEIDAVCGDFE
EHLGKIPHVGRRLVVFLGSTIGNLTPAPRAEFLSTLADTLQPGDSLLLGT
DLVKDTGRLVRAYDDAAGVTAAFNRNVLAVVNRELSADFDLDAFEHVAKW
NSDEERIEVWLRARTAQHVRVAALDLEVDFAAGEEMLTAVSCKFRPENVV
AELAEAGLRQTHWWTDPAGDFGLSLAVR*

Seq. ID No: 540

>TrpM_4MRVSGANHLGEDAGHLALRRDVYSGLQKTPKSLPPKWFYDTVG
SELFDQITRLPEYYPTRAEAEILRARSAEVASACRADTLVELGSGTSEKT
RMLLDALRHRGSLRRFVPFDVDASVLSATATAIQREYSGVEINAVCGDFE
EHLTEIPRGGRRLFVFLGSTIGNLTPGPRAQFLTALAGVMRPGDSLLLGT
DLVKDAARLVRAYDDPGGVTAQFNRNVLAVINRELEADFDVDAFQHVARW
NSAEERIEVWLRADGRQRVRVGALDLTVDFDAGEEMLTAVSCKFRPQAVG
AELAAAGLHRIRWWTDEAGDFGLSLAAK*

Seq. ID No: 541

>TrpM_5MTLTLSNYLAADSAATALRRDVHEGLTQSPKMLPPKWFYDSVG
SDLFDQITRLPEYYPTRTEAQILTHRSPEIVAAAGADTLVELGSGTSEKT
RMLLDAMRDGGQLRRFIPFDVDAGVLRAAGAAIGQEYPGIEIDAVCGDFE
EHLGKIPAVGRRLVAFLGSTIGNLTPGPRADFLASLAETLQPGDSVLLGT
DLVKDTGRLVSAYDDSAGVTAAFNRNVLSVVNRELDADFDLDAFAHVAKW
NAEEERIEVWLRADAPQQVRIAGLDLDVAFGAGEEMLTAVSCKFRADGVA
DELAKAGLRQTHWWTDEAGDFGLSLAVK*

Seq. ID No: 542

>TrpM_6MLEATSTQNLVSFQIPIVDIRTPSCLEETIRKKVVSGLARPYN
KKSIPDLLLYNETGLRLFEDLTYQPDYYLTGLEIEILSKHSLQIADSIPV
GSLIMELGAGALRKTALILDALEAQKKEVAYLALDLDRPELVRTLGQLNG
KYTHVKLGGLWGTYDDGRRWLSENTSDSPRTILWLGSSIGNVKRDDAGDF
IRSFGDVLSSKDRFVVAIDSRYHEVDTICRAYNDREGFAERFCLNGIDSF
NQLFGRAIIDISCAKYRTVYNEVKGRHEVYYRCTHDFEIRLPGDYPPTFL
YEGELILLAHSYKYAAVERETLWLRAGARPEKEWMTDGSYTVTMLSWP*

Seq. ID No: 543

>TrpM_7MSPSTVNKIASSPVFDIRSDETKGFAKAPIEDELAGLQAVYNE
KTLPNVLLYDAKGLQLFEKITYTNDYYLTGLEMDLLGEHADEMAEWIKDG
AALVELGAGALRKTAILLDAIERQGKRITFYALDLDHSELTRTLAELEGR
YRHITLCGLWGTYDDGRAWLASTNEEQRVLLWLGSSIGNLSRQEAKDFLH
SFGRALRPGIDKFIVAMDSKYNAVSSMTRAYNDSEGVTASFALNLLDAFN
AKVGFKALPPSSFCYSPFFNQAQGRNEAYLRARHGVRFEVNGIAVEVRDE
ELIRFAYSHKYDNAERDLLWRAAEANVEQEWLHSPQSGRARYSISLLSFR
D*

Seq. ID No: 544

>TrpM_8MTLSLSNHLPANSAARVLRRDVLDGLTQTPKALPPKWFYDSVG
SDLFDQITRLPEYYPTRTEAQILRTRSAEIAEASGADTLVELGSGTSEKT
RMLLDALRDNGTLRRFIPFDVDAGVLNAAGAAIQKEYPGVEVDAVCGDFE
EHLGEIPRVGRRLIAFLGSTIGNLTPQPRARFLTALAQTMRPGDSLLLGT
DLVKDTERLVRAYDDSAGVTARFNRNVLAVINRELDADFDLAAFDHVARF
NAAEERIEVWLRARGAQRVYVRELDLTVDFADGEEMLTAVSCKFRPDGVA
AELAAAGLRRTHWWTDPAGDFGLSLSTK*

Seq. ID No: 545

>TrpM_9MTISIANYLAADSAATALRRDVREGLAGTPKSLPPKWFYDSVG
SDLFDQITRLPEYYPTRAEAQILRTHAVDVAAASGADTLVELGSGTSEKT
RLLLDALHRADSLRRFIPFDVDASILQSAGAAISQEYPDVEIEAVCGDFE
EHLGKIPLQGRRLVVFLGSTIGNLTSGPRATFLSALADSLQPGDTLLLGT
DLVKDVDRLKRAYDDAAGVTARFNKNVLTVVNRELGADFDLDAFEHVCKW
NADEERIEVWLRANTLQRVHISGLELDVEYAAGEEMLTAVSCKFRPEGIA
AELAAVGLNRTHWWTDDAGDFGLSLAVK*

Seq. ID No: 546

>TrpS_1MTTLLNPYFGEFGGMYVPQILMPALNQLEEAFVSAQKDPEFQA
QFADLLKNYAGRPTALTKCQNITAGTRTTLYLKREDLLHGGAHKTNQVLG
QALLAKRMGKSEIIAETGAGQHGVASALASALLGLKCRIYMGAKDVERQS
PNVFRMRLMGAEVIPVHSGSATVKDACNEALRDWSGSYETAHYMLGTAAG
PHPYPTIVREFQRMIGEETKAQILDKEGRLPDAVIACVGGGSNAIGMFAD
FINDTSVGLIGVEPGGHGIETGEHGAPLKHGRVGIYFGMKAPMMQTADGQ
IEESYSISAGLDFPSVGPQHAYLNSIGRADYVSITDDEALEAFKTLCRHE
GIIPALESSHALAHALKMMREQPEKEQLLVVNLSGRGDKDIFTVHDILKA
RGEI

Seq. ID No: 547

>TrpS_2MWFGEFGGQYVLETLIGPLKELEKAYKRFKDDEEFNRQLNYYL
KTWAGRPTPLYYAKRLTEKIGGAKVYLKREDLVHGGAHKTNNAIGQALLA
KFMGKTRLIAETGAGQHGVATAMAGALLGMKVDIYMGAEDVERQKMNVFR
MKLLGANVIPVNSGSRTLKDAINEALRDWVATFEYTHYLIGSVVGPHPYP
TIVRDFQSVIGREAKAQILEAEGQLPDVIVACVGGGSNAMGIFYPFVNDK
KVKLVGVEAGGKGLESGKHSASLNAGQVGVSHGMLSYFLQDEEGQIKPSH
SIAPGLDYPGVGPEHAYLKKIQRAEYVAVTDEEALKAFHELSRTEGIIPA
LESAHAVAYAMKLAKEMSRDEIIIVNLSGRGDKDLDIVLKVSGNV

Seq. ID No: 548

>affibody_tag_1MVDNKFNKETIQASQEIRLLPNLNGRQKLAFIHSL
LDDPSQSANLLAEAKKLNDAQAPKNAAIRSSSASSGGSGGSSSS

Seq. ID No: 549

>affibody_tag_2NAAIRSSSASSGGSGGSSSSVDNKFNKETIQASQE
IRLLPNLNGRQKLAFIHSLLDDPSQSANLLAEAKKLNDAQAPK

Seq. ID No: 550

>affibody_tag_3NAAIRSSSASSGGSGGSSSSGVDNKFNKELGWATW
EIFNLPNLNGVQVKAFIDSLRDDPSQSANLLAEAKKLNDAQAPK

Seq. ID No: 551

>affibody_tag_4MVDNKFNKELGWATWEIFNLPNLNGVQVKAFIDSL
RDDPSQSANLLAEAKKLNDAQAPKGNAAIRSSSASSGGSGGSSSS

Seq. ID No: 552

>affibody_tag_5MVDNKFNKEMRNAYWEIALLPNLNNQQKRAFIRSL
YDDPSQSANLLAEAKKLNDAQAPKSSNAAIRSSSASSGGSGGSSSS

Seq. ID No: 553

>affibody_tag_6NAAIRSSSASSGGSGGSSSSGVDNKFNKEMRNAYW
EIALLPNLNNQQKRAFIRSLYDDPSQSANLLAEAKKLNDAQAPK

Seq. ID No: 554

>cofold_1MKIEEGKLVIWINGDKGYNGLAEVGKKFEKDTGIKVTVEHP
DKLEEKFPQVAATGDGPDIIFWAHDRFGGYAQSGLLAEITPDKAFQDKLY
PFTWDAVRYNGKLIAYPIAVEALSLIYNKDLLPNPPKTWEEIPALDKELK
AKGKSALMFNLQEPYFTWPLIAADGGYAFKYENGKYDIKDVGVDNAGAKA
GLTFLVDLIKNKHMNADTDYSIAEAAFNKGETAMTINGPWAWSNIDTSKV
NYGVTVLPTFKGQPSKPFVGVLSAGINAASPNKELAKEFLENYLLTDEGL
EAVNKDKPLGAVALKSYEEELAKDPRIAATMENAQKGEIMPNIPQMSAFW
YAVRTAVINAASGRQTVDEALKDAQTNSSSNNNNNNNNNNLGIEGR

Seq. ID No: 555

>cofold_2MVSKGEELFTGVVPILVELDGDVNGHKFSVSGEGEGDATYG
KLTLKLICTTGKLPVPWPTLVTTLGYGLQCFARYPDHMKQHDFFKSAMPE
GYVQERTIFFKDDGNYKTRAEVKFEGDTLVNRIELKGIDFKEDGNILGHK
LEYNYNSHNVYITADKQKNGIKANFKIRHNIEDGGVQLADHYQQNTPIGD
GPVLLPDNHYLSYQSALSKDPNEKRDHMVLLEFVTAAGITLGMDELYK

Seq. ID No: 556

>cofold_3MAMFCTFFEKHHRKWDILLEKSTGVMEAMKVTSEEKEQLST
AIDRMNEGLDAFIQLYNESEIDEPLIQLDDDTAELMKQARDMYGQEKLNE
KLNTIIKQILSISVSEEGEKEGSGSG

Seq. ID No: 557

>cofold_4MYLLGIGLILALIACKQNVSSLDEKNSVSVDLPGEMKVLVS
KEKNKDGKYDLIATVDKLELKGTSDKNNGSGVLEGVKADKSKVKLTISDD
GSG

Seq. ID No: 558

>cofold_5MADRDRSGIYGGAHATYGQQQQQGGGGRPMGEQVKGMLHDK
GPTASQALTVATLFPLGGLLLVLSGLALTASVVGLAVATPVFLIFSPVLV
PAALLIGTAVMGFLTSGALGLGGLSSLTCLANTARQAFQRTPDYVEEAHR
RMAEAAAHAGHKTAQAGQAIQGRAQEAGAGGGAG

Seq. ID No: 559

>oxidase_1MKILILGIFLFLCSTPAWAKEKHYYIGIIETTWDYASDHG
EKKLISVDTEHSNIYLQNGPDRIGRLYKKALYLQYTDETFRTTIEKPVWL
GFLGPIIKAETGDKVYVHLKNLASRPYTFHSHGITYYKEHEGAIYPDNTT
DFQRADDKVYPGEQYTYMLLATEEQSPGEGDGNCVTRIYHSHIDAPKDIA
SGLIGPLIICKKDSLDKEKEKHIDREFVVMFSVVDENFSWYLEDNIKTYC
SEPEKVDKDNEDFQESNRMYSVNGYTFGSLPGLSMCAEDRVKWYLFGMGN
EVDVHAAFFHGQALTNKNYRIDTINLFPATLFDAYMVAQNPGEWMLSCQN
LNHLKAGLQAFFQVQECNKSSSKDNIRGKHVRHYYIAAEEIIWNYAPSGI
DIFTKENLTAPGSDSAVFFEQGTTRIGGSYKKLVYREYTDASFTNRKERG
PEEEHLGILGPVIWAEVGDTIRVTFHNKGAYPLSIEPIGVRFNKNNEGTY
YSPNYNPQSRSVPPSASHVAPTETFTYEWTVPKEVGPTNADPVCLAKMYY
SAVDPTKDIFTGLIGPMKICKKGSLHANGRQKDVDKEFYLFPTVFDENES
LLLEDNIRMFTTAPDQVDKEDEDFQESNKMHSMNGFMYGNQPGLTMCKGD
SVVWYLFSAGNEADVHGIYFSGNTYLWRGERRDTANLFPQTSLTLHMWPD
TEGTFNVECLTTDHYTGGMKQKYTVNQCRRQSEDSTFYLGERTYYIAAVE
VEWDYSPQREWEKELHHLQEQNVSNAFLDKGEFYIGSKYKKVVYRQYTDS
TFRVPVERKAEEEHLGILGPQLHADVGDKVKIIFKNMATRPYSIHAHGVQ
TESSTVTPTLPGETLTYVWKIPERSGAGTEDSACIPWAYYSTVDQVKDLY
SGLIGPLIVCRRPYLKVFNPRRKLEFALLFLVFDENESWYLDDNIKTYSD
HPEKVNKDDEEFIESNKMHAINGRMFGNLQGLTMHVGDEVNWYLMGMGNE
IDLHTVHFHGHSFQYKHRGVYSSDVFDIFPGTYQTLEMFPRTPGIWLLHC
HVTDHIHAGMETTYTVLQNEDTKSG

Seq. ID No: 560

>oxidase_2MGLNSAIPSLAILALSVGSYAAIGPVSDLHIVNKDLAPDG
VQRPTVLAGGTFPGTLITGQKGDNFQLNVIDDLTDDRMLTPTSIHWHGFF
QKGTAWADGPAFVTQCPIIADNSFLYDFDVPDQAGTFWYHSHLSTQYCDG
LRGAFVVYDPNDPHKDLYDVDDESTVITLADWYHVLAQTVVGAATPDSTL
INGLGRSQTGPADAELAVISVEHNKRYRFRLVSISCDPNFTFSIDGHNMT
VIEVDGVNTRPLTVDSIQIFAGQRYSFVLNANQPDDNYWIRAMPNIGRNT
TTLDGKNAAILRYKNASVEEPKTVGGPAQSPLNEADLRPLVPAPVPGNAV
PGGADINHRLNLTFSNGLFSINNASFTNPSVPALLQILSGAQNAQDLLPT
GSYIGLELGKVVELVIPPLAVGGPHPFHLHGHNFWVVRSAGSDEYNFDDA
ILRDVVSIGAGTDEVTIRFVTDNPGPWFLHCHIDWHLEAGLAIVFAEGIN
QTAAANPTPQAWDELCPKYNGLSASQKVKPKKGTAI

Seq. ID No: 561

>oxidase_3MSRFQSLLSFVLVSLAAVANAAIGPVADLTLTNAAVSPDG
FSREAVVVNGITPAPLIAGQKGDRFQLNVIDNLTNHTMLKTTSIHWHGFF
QHGTNWADGVSFVNQCPIASGHSFLYDFQVPDQAGTFWYHSHLSTQYCDG
LRGPFVVYDPNDPQASLYDIDNDDTVITLADWYHVAAKLGPRFPLGADAT
LINGLGRSPGTTTADLAVIKVTQGKRYRFRLVSLSCDPNHTFSIDGHTMT
VIEADSVNTQPLEVDSIQIFAAQRYSFVLDASQPVDNYWIRANPAFGNVG
FAGGINSAILRYDGAPEVEPTTTQTTSTKPLNEADLHPLTPMPVPGRPEA
GGVDKPLNMVFNFNGTNFFINNHSFVPPSVPVLLQILSGAQAAQDLVPDG
SVYVLPSNSSIEISFPATANAPGTPHPFHLHGHTFAVVRSAGSSEYNYDN
PIFRDVVSTGQPGDNVTIRFQTNNPGPWFLHCHIDFHLEAGFAVVLAEDT
PDTAAVNPVPQSWSDLCPIYDALDPSDL

Seq. ID No: 562

>oxidase_4MKFLLLSALLFLHSSLAWTREKHYYIGITEAVWDYASGSE
EKELISVDTEQSNFYLRNGPDRIGRKYKKALYSEYTDGTFTKTIDKPAWL
GFLGPVIKAEVGDKVSVHVKNFASRPYTFHAHGVTYTKANEGAIYPDNTT
DFQRADDKLFPGQQYLYVLRANEPSPGEGDSNCVTRIYHSHVDAPKDIAS
GLIGPLILCKKGSLHKEKEENIDQEFVLMFSVVDENLSWYLEDNIKTFCS
EPEKVDKDNEDFQESNRMYSINGYTFGSLPGLSMCAEDRVKWYLFGMGNE
VDVHSELFHGQALTSKNYHTDIINLFPATLIDVSMVAQNPGVWMLSCQNL
NHLKAGLQAFFQVRDCNKPSPDDDIQDRHVRHYYIAAEETIWDYAPSGTD
TFTGENFTSLGSDSRVFFEQGATRIGGSYKKLVYREYTDDSFTNRKERGP
DEEHLGILGPVIWAEVGDIIRVTFHNKGQFPLSIQPMGVRFTKENEGTYY
GPDGRSSKQASHVAPKETFTYEWTVPKEMGPTYADPVCLSKMYYSGVDLT
KDIFTGLIGPMKICKKGSLLADGRQKDVDKEFYLFATVFDENESLLLDDN
IRMFTTAPENVDKEDEDFQESNKMHSMNGFMYGNLPGLNMCLGESIVWYL
FSAGNEADVHGIYFSGNTYLSKGERRDTANLFPHKSLTLLMTPDTEGSFD
VECLTTDHYTGGMKQKYTVNQCKGQFEDVTLYQGERTYYIAAVEVEWDYS
PSRDWEMELHHLQEQNVSNAFLDKEEFFIGSKYKKVVYREFTDSTFREQV
KRRAEEEHLGMLGPLIHADVGAKVKVVFKNMATRPYSIHAHGVKTKSSTV
APTLPGEVRTYIWQIPERSGAGTEDSPCIPWAYYSTVDRVKDLYSGLIGP
LIVCRKSYVKVFNPKKKMEFSLLFLVFDENESWYLDDNINTYPDHPEKDN
KDNEEFIESNKMHAINGKMFGNLQGLTMHVGDEVNWYVMAMGNEIDLHTV
HFHGHSFQYKHRGIHSSDVFDFFPGTYQTLEMFPQTPGTWLLHCHVTDHI
HAGMVTTYTVLPNQETKSG

Seq. ID No: 563

>oxidase_5MNFVTALPLIAQLIGTARAAIGPVTNLLVKNADIPPDGFT
RAAVVANNQFPGPVIRATKGDTLSLNVVNQLTDATMLMGTSIHWHGFHQK
GTSWADGVVGVTQCPIAPGHSFLYQFPTANQAGTFWYHSHYSTQYCDGLR
GALIVYDPTDPYRTWYDIDDESTIITLADWYHKAAPLQTLRTAKEDSVLI
NGQGRVPGDKTTDSTPLSVINIIPQKRYRFRLISISCDPAFSFSIDGHSM
TVIEADSQSVQPLTVNEITIFAGQRYSFILYANNPVGNYWIRSQPTYPDD
GIQGYAGGINSAILRYSGAPAVNPTTKKASITIPLVEADLRPLYSPAAPG
LPSPGAADVNIKLDISYNSPSETFFVNNSTFPEVPVPVLLQILSGAQSAN
DLLPAGSVYTLPPNKVIEISMPGGRPGSPHPMHLHGHDFSVVRSAGSNRY
NYANPVRRDVVNIGMEDTDNVTIRFRVCSHTYLSLHCHIDFHLEDGQSGT
LVPPLPHRLPPRGRIRCRLHRGILVRGRLGPDLQ

Seq. ID No: 564

>phosphatase_1MQGPWVLLLLGLRLQLSLGIIPVEEENPDFWNRQAA
EALGAAKKLQPAQTAAKNLIIFLGDGMGVSTVTAARILKGQKKDKLGPET
FLAMDRFPYVALSKTYSVDKHVPDSGATATAYLCGVKGNFQTIGLSAAAR
FNQCNTTRGNEVISVMNRAKKAGKSVGVVTTTRVQHASPAGAYAHTVNRN
WYSDADVPASARQEGCQDIATQLISNMDIDVILGGGRKYMFPMGTPDPEY
PDDYSQGGTRLDGKNLVQEWLAKHQGARYVWNRTELLQASLDPSVTHLMG
LFEPGDMKYEIHRDSTLDPSLMEMTEAALLLLSRNPRGFFLFVEGGRIDH
GHHESRAYRALTETIMFDDAIERAGQLTSEEDTLSLVTADHSHVFSFGGY
PLRGSSIFGLAPGKARDRKAYTVLLYGNGPGYVLKDGARPDVTESESGSP
EYRQQSAVPLDGETHAGEDVAVFARGPQAHLVHGVQEQTFIAHVMAFAAC
LEPYTACDLAPRAGTTDAAHPGPSVVPALLPLLAGTLLLLGTATAP

Seq. ID No: 565

>phosphatase_2MQGACVLLLLGLHLQLSLGLVPVEEEDPAFWNRQAA
QALDVAKKLQPIQTAAKNVILFLGDGMGVPTVTATRILKGQMNGKLGPET
PLAMDQFPYVALSKTYNVDRQVPDSAGTATAYLCGVKGNYRTIGVSAAAR
YNQCKTTRGNEVTSVMNRAKKAGKSVGVVTTTRVQHASPAGAYAHTVNRN
WYSDADLPADAQMNGCQDIAAQLVNNMDIDVILGGGRKYMFPVGTPDPEY
PDDASVNGVRKRKQNLVQAWQAKHQGAQYVWNRTALLQAADDSSVTHLMG
LFEPADMKYNVQQDHTKDPTLQEMTEVALRVVSRNPRGFYLFVEGGRIDH
GHHDDKAYMALTEAGMFDNAIAKANELTSELDTLILVTADHSHVFSFGGY
TLRGTSIFGLAPSKALDSKSYTSILYGNGPGYALGGGSRPDVNDSTSEDP
SYQQQAAVPQASETHGGEDVAVFARGPQAHLVHGVEEETFVAHIMAFAGC
VEPYTDCNLPAPTTATSIPDAAHLAASPPPLALLAGAMLLLLAPTLY

Seq. ID No: 566

>phosphatase_3MQGPWVLLLLGLRLQLSLGVIPAEEENPAFWNRQAA
EALDAAKKLQPIQKVAKNLILFLGDGLGVPTVTATRILKGQKNGKLGPET
PLAMDRFPYLALSKTYNVDRQVPDSAATATAYLCGVKANFQTIGLSAAAR
FNQCNTTRGNEVISVMNRAKQAGKSVGVVTTTRVQHASPAGTYAHTVNRN
WYSDADMPASARQEGCQDIATQLISNMDIDVILGGGRKYMFPMGTPDPEY
PADASQNGIRLDGKNLVQEWLAKHQGAWYVWNRTELMQASLDQSVTHLMG
LFEPGDTKYEIHRDPTLDPSLMEMTEAALRLLSRNPRGFYLFVEGGRIDH
GHHEGVAYQALTEAVMFDDAIERAGQLTSEEDTLTLVTADHSHVFSFGGY
TLRGSSIFGLAPSKAQDSKAYTSILYGNGPGYVFNSGVRPDVNESESGSP
DYQQQAAVPLSSETHGGEDVAVFARGPQAHLVHGVQEQSFVAHVMAFAAC
LEPYTACDLAPPACTTDAAHPVAASLPLLAGTLLLLGASAAP

Seq. ID No: 567

>phosphatase_4MQGAWVLLLLGFRLQLSLSVIPVEEENPAFWTQKAA
DALNVAKKLQPIQTSAKNLIIFLGDGMGVATVTATRILKGQLEGNLGPET
PLAMDHFPYMALSKTYSVDRQVPDSASTATAYLCGVKTNYKTIGVSAAAR
FDQCNTTFGNEVLSVMYRAKKAGKSVGVGDHTRVQHASPAGTYVHTVTSN
WYGDADMPALPLQEGCKDIATQLISNMDINVILGGGRKYMFPAGTPDPEY
PNDVNETGTRLDGKNLVQEWLSKHQGSQYVWNRQELIQKSLDPSVTYLMG
LFEPVDTKFEIQRDPLMDPSLKDMTEAALHVLSRNPKGFYLFVEGGRIDR
GHHLGTAYLALTEAVMFDSAIERASLQASEQDTLTIVTADHSHVFSFGGY
TLRGTSIFGLAPLNALDGKPYTSILYGNGPGYVGTGERPNVTDAESHDPS
YQQQAAVPVKSETTVGKDVAIFARGPQAHLLHGVQEQNYIAHVMAFAGCL
EPYTDCGLAPPADENRPTTPVQNSTTTTTTTTTTTTTTTTTRVQNSASSL
GPATAPLAWHYWPRR

Seq. ID No: 568

>phosphatase_5MSGSSVTGGGASLPAELYKGSADSILPANFSYAVTG
SGTGKNAFLTNNSSLFGTTGTVHYAGSDSVLSGSELTTYNSNYNGTYGPL
IQIPSVATSVTVPYRKDGNTTLNLTSAQLCDAFSGAKTTWGQLLGTTDST
PIRIVYRTGSSGTTELFTRHLNSICPTRFATNSTFTNARLPAGGTLPSNW
VGVAATSTVVSTVKATNGSLGYVSPDAVNINSNAEVSRVNGNLPTQANVS
TALGSVAPPANAADRADPSKWVPVFTNPSAGYSIVGYTNFVFGQCYKDAS
VSTDVRAFINKHYGGTTTNAAVAAHGFIPLTPAWKSAIVSAFYTGTSENL
AIGNTNVCNTKGRP

Seq. ID No: 569

>phosphatase_6MNFVTALPLIAQLIGTARAAIGPVTNLLVKNADIPP
DGFTRAAVVANNQFPGPLITGNQGINFQINVVARLNNECSSRPLWYHWHG
FFQKGTNWADGPAFVNQCPISTGNSFLYDFTAADQAGTFWYHSHLSTQYC
DGLRGPMVVYDPNDPHASLYDVDDESTVITLSDWYHTAARLGSAFPLGPD
SVLINGLGRFAGGDGNAELAVIPVTQGKRYRFRLISLSCDPNFVFSIDKH
NMTVIEADAVSHEPVTVDSIHIYAGQRYSFVLSAHRDIDNYWIRALPSGG
TVNFVGGVNSALIRYDGAAEVEPVTNTTMSIAPLVETDLVPLDSPAAPGE
ASIGGVDYALSLVPSFVSRTLFCVRSIADDLRLRTGRTILSGSTELPSSH
PPCRVYTLPSNATIELSFPITATNAPGAPHPFHLHGHVFSVVRSAGSSEY
NYANPPRRDVVNTGTAGDNVTIRFRVCSHTYLSLHCHIDFHLEDGQSGTL
VPPLPHRLPPRGRIRCRLHRGILVRGRLGPDLQ

Seq. ID No: 570

>scaffoldGGASASGVDNKFNKERVIAIGEIMRLPNLNSLQVVAFINSL
RDDPSQSANLLAEAKKLNDAQAPKGGSSASSAGGSSVDNKFNKEAQTAGV
EIMELPNLNTRQLLAFIQSLRDDPSQSANLLAEAKKLNDAQAPKTSGGSS
ASSAGGSSVDNKFNKERRMAAYEIIDLPNLNWFQLEAFITSLSDDPSQSA
NLLAEAKKLNDAQAPKGSGSNAAIRSSGSGSVDNKFNKERVIAIGEIMRL
PNLNSLQVVAFINSLRDDPSQSANLLAEAKKLNDAQAPKGGSSASSAGGS
SVDNKFNKEAQTAGVEIMELPNLNTRQLLAFIQSLRDDPSQSANLLAEAK
KLNDAQAPKTSGSGSANAAIRSAGSGSVDNKFNKERRMAAYEIIDLPNLN
WFQLEAFITSLSDDPSQSANLLAEAKKLNDAQAPKGSGASGSGAGGSSVD
NKFNKERVIAIGEIMRLPNLNSLQVVAFINSLRDDPSQSANLLAEAKKLN
DAQAPKGSGASGSGAGGSSVDNKFNKEAQTAGVEIMELPNLNTRQLLAFI
QSLRDDPSQSANLLAEAKKLNDAQAPKTSGSGSANAAIRSAGSGSVDNKF
NKERRMAAYEIIDLPNLNWFQLEAFITSLSDDPSQSANLLAEAKKLNDAQ
APK

Seq. ID No: 571

>sec_1MQLLRCFSIFSVIASVLAQELTTICEQIPSPTLESTPYSLSTTT
ILANGK

Seq. ID No: 572

>sec_2MLSLKTLLCTLLTVSSVLATPVPARDPSSIQFVHEENKKRYYDY
DHGSLGE

Seq. ID No: 573

>sec_3MKLQSLLVSAAVLTSLTENVNAWSPNNSYVPANVTCDDDINLVR
EASGLSDNETEWLKKRDAYTKE

Seq. ID No: 574

>sec_4MEGVSLEKREAEA

Seq. ID No: 575

>sec_5MKKTAIAIAVALAGFATVAQA

Seq. ID No: 576

>vac_1MFSLKALLPLALLLVSANQVAAKVHKAKIYKHELS

Seq. ID No: 577

>vac_2MTKNFIVTLKKNTPDVEAKKFLDSVHHAGGSIVHKFDIIKGYTI
KVPDVLHLNKLKEKHNDVIENVEEDKEVHTN

Seq. ID No: 578

>vac_3MEEQREILEQLKKTLQMLTVEPSKNNQIANEEKEKKENENSWCI
LEHNYEDIAQEFIDFIYKNPTTYHVVSFFAELLDKHNFKYLSEKSNWQDS
IGEDGG

What is claimed is:

1. A non-naturally occurring nucleic acid comprising a sequence encoding an enzyme or regulatory protein in tryptamine metabolism,
wherein the enzyme or regulatory protein is an N-methyltransferase (INMT, PsiM, TrpM), a tryptophan decarboxylase (AADC), a tryptophan hydroxylase (TPH), a tryptamine 4′ hydroxylase (T4H), a tryptamine 5′ hydroxylase (T5H), a truncated cytochrome p450 reductase (T4H-CPR, T5H-CPR), an hydroxytryptamine O-methyltransferase (IOMT or CaffOMT), an N-acetyltransferase (NAT), a deacetylase (DAC), a hydroxyl tryptamine kinase (PsiK), a tryptophan synthase (TrpS), a toluene monooxygenase (TMO), an aminotransferase/methyltransferase fusion (ATMT), a phosphatase, an oxidase, a dimethylallyltryptophan synthase (DMATS), an isopentenyl-diphosphate isomerase (IDI1), a tryptophan halogenase (TrpHalo), an aspartate oxidase/quinolinic acid synthase fusion (AOQS), a tryptophan importer (TAT2), a methionine importer (MUP1), or a SAMe importer (SAM3).

2. The nucleic acid of claim 1, encoding a methyltransferase or hydroxylase, wherein the methyltransferase or hydroxylase is a tryptamine N-methyltransferase (INMT), a hydroxytryptamine O-methyltransferase (IOMT), a tryptamine 5′ hydroxylase (T5H) or a tryptophan N-methyltransferase (TrpM).

3-5. (canceled)

6. The nucleic acid of claim 1, wherein the sequence is codon-optimized for yeast expression.

7. (canceled)

8. The nucleic acid of claim 1, having a 5′ end, further comprising additional nucleotides at the 5′ end that encode a codon optimized cofolding peptide.

9. The nucleic acid of claim 8, wherein the codon optimized cofolding peptide comprises an amino acid sequence of any one of SEQ ID NO:554-558.

10. The nucleic acid of claim 9, wherein the codon optimized cofolding peptide is encoded by any one of SEQ ID NOs:265-269.

11. (canceled)

12. The nucleic acid of claim 1, comprising the sequence of any one of SEQ ID NOs:1-289.

13. The nucleic acid of claim 1, further comprising a promoter functional in a yeast.

14. (canceled)

15. A yeast expression cassette comprising the nucleic acid of claim 13.

16. (canceled)

17. A recombinant microorganism yeast comprising the expression cassette of claim 15, that expresses the enzyme or regulatory protein encoded therein.

18-21. (canceled)

22. A non-naturally occurring enzyme or regulatory protein comprising an amino acid sequence encoded by the nucleic acid of claim 1.

23-27. (canceled)

28. The recombinant yeast of claim 17, expressing INMT, wherein the recombinant microorganism produces at least one hydroxy substituted tryptophan compound.

29. The recombinant microorganism yeast of claim 28, wherein the at least one hydroxy substituted tryptophan compound is 5-OH-NMTP, 5-OH-DMTP or 5-OH-TMTP.

30. The recombinant yeast of claim 17, expressing INMT, wherein the recombinant microorganism produces at least one hydroxy substituted tryptamine compound.

31. The recombinant microorganism yeast of claim 30, wherein the at least one hydroxy substituted tryptamine compound is bufotenine, 5-OH-NMT, or 5-OH-TMT.

32. The recombinant yeast of claim 17, expressing INMT, wherein the recombinant microorganism produces at least one methoxy substituted tryptamine compound.

33. The recombinant microorganism yeast of claim 32, wherein the at least one methoxy substituted tryptamine compound is 5-MeO-NMT, 5-MeO-DMT, or 5-MeO-TMT.

34. The recombinant yeast of claim 17, expressing IOMT, wherein the recombinant microorganism methylates the primary amine on the 5-hydroxy moiety on an indole ring.

35. The recombinant microorganism yeast of claim 34, wherein the microorganism acts on (a) bufotenine to create 5-MeO-DMT, or (b) N-acetylserotonin to create melatonin.

36. The recombinant yeast of claim 17, expressing T5H, wherein the recombinant microorganism hydroxylates at the 5′ position of an indole ring.

37-111. (canceled)
